SEQUENCE LISTING (2 sequences)

SEQ ID NO: 1 - protein, 20 aa, Artificial Sequence
  synthetic
GGDYKDDDDKGGHHHHHHHH

SEQ ID NO: 2 - protein, 29 aa, Artificial Sequence
  synthetic
DYKDDDDKHHHHHHGLNDIFEAQKIEWHE

(19) United States
(12) Patent Application Publication
(10) Pub. No.: US 2023/0086703 A1
(43) Pub. Date: Mar. 23, 2023

(54) N-(4-(5-chloropyridin-3-yl)phenyl)-2-(2-(cyclopropanesulfonamido)pyrimidin-4-yl) butanamide derivatives and related compounds as human CTPS1 inhibitors for the treatment of proliferative diseases

(71) Applicant: Step Pharma S.A.S., Paris (FR)

(72) Inventors: Abdul Quddus, Nottingham, Nottinghamshire (GB); Andrew Novak, Nottingham, Nottinghamshire (GB); David Cousin, Nottingham, Nottinghamshire (GB); Emma Blackham, Nottingham, Nottinghamshire (GB); Ellie Chatzopoulou, Nottingham, Nottinghamshire (GB); Geraint Jones, Nottingham, Nottinghamshire (GB); Jennifer Thomas, Nottingham, Nottinghamshire (GB); Joseph Wrigglesworth, Nottingham, Nottinghamshire (GB); Lorna Duffy, Nottingham, Nottinghamshire (GB); Louise Birch, Nottingham, Nottinghamshire (GB); Pascal George, Paris (FR); Saleh Ahmed, Nottingham, Nottinghamshire (GB); Gianna Toschi, Nottingham, Nottinghamshire (GB)

(73) Assignee: Step Pharma S.A.S., Paris (FR)

(21) Appl. No.: 17/615,879

(22) PCT Filed: Jun. 4, 2020
(86) PCT No.: PCT/IB2020/000560

§ 371 Date: Dec. 2, 2021

(30) Foreign Application Priority Data
Jun. 4, 2019 (EP) 19305714.8
Jun. 4, 2019 (EP) 19305715.5
Jun. 4, 2019 (EP) 19305717.1

(51) Int. Cl. C07D 417/12; C07D 277/28; C07D 417/14

(52) U.S. Cl. CPC C07D 417/12; C07D 277/28; C07D 417/14

(57) ABSTRACT

Compounds of formula (I) wherein ring B is selected from the group consisting of Formula (B-a) and Formula (B-bc) as human cytidine triphosphate synthase 1 (CTPS 1) inhibitors for the treatment of proliferative diseases, such as e.g. cancer, such as e.g. leukemia and lymphoma, e.g. inflammatory skin diseases such as psoriasis, or e.g. multiple sclerosis. The present description discloses the synthesis and characterisation of exemplary compounds as well as pharmacological data thereof (e.g. page 129 to page 302; examples; biological examples 1 and 2; tables 1-17). Specific examples are e.g.: N-(4-(5-Chloropyridin-3-yl)phenyl)-2-(2-(cyclopropane-sulfonamido) pyrimidin-4-yl)butanamide (Formula P1) or 1-(2-(Cyclopropanesulfonamido)pyrimidin-4-yl)-N-(4-(6-ethoxypyrazin-2-yl)phenyl)cyclopentanecarboxamide (Formula P2).

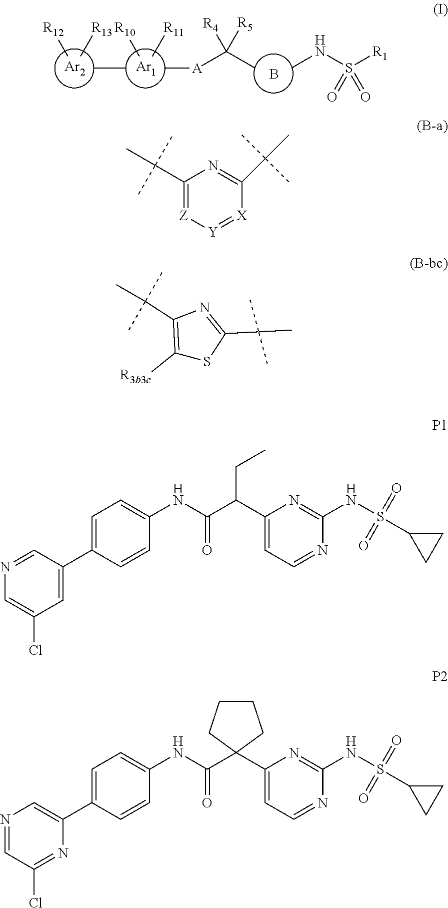

### FIELD OF THE INVENTION

The invention relates to novel compounds, processes for the manufacture of such compounds, related intermediates, compositions comprising such compounds and the use of such compounds as cytidine triphosphate synthase 1 inhibitors, particularly in the treatment or prophylaxis of disorders associated with cell proliferation.

### BACKGROUND OF THE INVENTION

Nucleotides are a key building block for cellular metabolic processes such as deoxyribonucleic acid (DNA) and ribonucleic acid (RNA) synthesis. There are two classes of nucleotides, that contain either purine or pyrimidine bases, both of which are important for metabolic processes. Based on this, many therapies have been developed to target different aspects of nucleotide synthesis, with some inhibiting generation of purine nucleotides and some pyrimidine nucleotides or both.

The pyrimidine nucleotide cytidine 5′ triphosphate (CTP) is a precursor required not just for the anabolism of DNA and RNA but also phospholipids and sialyation of proteins. CTP originates from two sources: a salvage pathway and a de novo synthesis pathway that depends on two enzymes, the CTP synthases (or synthetases) 1 and 2 (CTPS1 and CTPS2) (Evans and Guy 2004; Higgins, et al. 2007; Ostrander, et al. 1998).

CTPS1 and CTPS2 catalyse the conversion of uridine triphosphate (UTP) and glutamine into cytidine triphosphate (CTP) and L-glutamate:

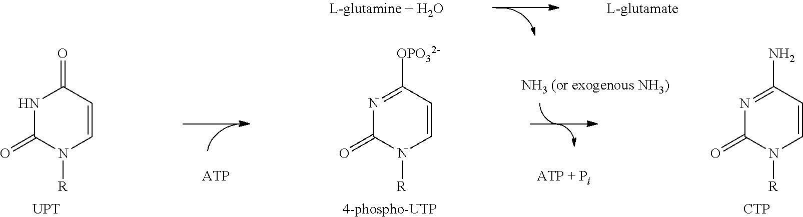

Both enzymes have two domains, an N-terminal synthetase domain and a C-terminal glutaminase domain (Kursula, et al. 2006). The synthetase domain transfers a phosphate from adenosine triphosphate (ATP) to the 4-position of UTP to create an activated intermediate, 4-phospho-UTP. The glutaminase domain generates ammonia from glutamine, via a covalent thioester intermediate with a conserved active site cysteine, generating glutamate. This ammonium is transferred from the glutaminase domain to the synthetase domain via a tunnel or can be derived from external ammonium. This ammonium is then used by the synthetase domain to generate CTP from the 4-phospho-UTP (Lieberman, 1956).

Although CTPS exists as two isozymes in humans and other eukaryotic organisms, CTPS1 and CTPS2, functional differences between the two isozymes are not yet fully elucidated (van Kuilenburg, et al. 2000).

The immune system provides protection from infections and has therefore evolved to rapidly respond to the wide variety of pathogens that the individual may be exposed to. This response can take many forms, but the expansion and differentiation of immune populations is a critical element and is hence closely linked to rapid cell proliferation. Within this, CTP synthase activity appears to play an important role in DNA synthesis and the rapid expansion of lymphocytes following activation (Fairbanks, et al. 1995; van den Berg, et al. 1995).

Strong clinical validation that CTPS1 is the critical enzyme in human lymphocyte proliferation came with the identification of a loss-of-function homozygous mutation (rs145092287) in this enzyme that causes a distinct and life-threatening immunodeficiency, characterized by an impaired capacity of activated T- and B-cells to proliferate in response to antigen receptor-mediated activation. Activated CTPS1-deficient cells were shown to have decreased levels of CTP. Normal T-cell proliferation was restored in CTPS1-deficient cells by expressing wild-type CTPS1 or by addition of cytidine. CTPS1 expression was found to be low in resting lymphocytes, but rapidly upregulated following activation of these cells. Expression of CTPS1 in other tissues was generally low. CTPS2 seems to be ubiquitously expressed in a range of cells and tissues but at low levels, and the failure of CTPS2, which is still intact in the patients, to compensate for the mutated CTPS1, supports CTPS1 being the critical enzyme for the immune populations affected in the patients (Martin, et al. 2014).

Overall, these findings suggest that CTPS1 is a critical enzyme necessary to meet the demands for the supply of CTP required by several important immune cell populations.

Normally the immune response is tightly regulated to ensure protection from infection, whilst controlling any response targeting host tissues. In certain situations, the control of this process is not effective, leading to immune-mediated pathology. A wide range of human diseases are thought to be due to such inappropriate responses mediated by different elements of the immune system.

Given the role that cell populations, such as T and B lymphocytes, are thought to play in a wide range of autoimmune and other diseases, CTPS1 represents a target for a new class of immunosuppressive agents. Inhibition of CTPS1 therefore provides a novel approach to the inhibition of activated lymphocytes and selected other immune cell populations such as Natural Killer cells, Mucosal-Associated Invariant T (MAIT) and Invariant Natural Killer T cells, highlighted by the phenotype of the human mutation patients (Martin, et al. 2014).

Cancer can affect multiple cell types and tissues but the underlying cause is a breakdown in the control of cell division. This process is highly complex, requiring careful coordination of multiple pathways, many of which remain to be fully characterised. Cell division requires the effective replication of the cell's DNA and other constituents. Interfering with a cell's ability to replicate by targeting nucleic acid synthesis has been a core approach in cancer therapy for many years. Examples of therapies acting in this way are 6-thioguanine, 6-mecaptopurine, 5-fluorouracil, cytarabine, gemcitabine and pemetrexed.

As indicated above, pathways involved in providing the key building blocks for nucleic acid replication are the purine and pyrimidine synthesis pathways, and pyrimidine biosynthesis has been observed to be up-regulated in tumors and neoplastic cells.

CTPS activity is upregulated in a range of tumour types of both haematological and non-haematological origin, although heterogeneity is observed among patients. Linkages have also been made between high enzyme levels and resistance to chemotherapeutic agents.

Currently, the precise role that CTPS1 and CTPS2 may play in cancer is not completely clear. Several non-selective CTPS inhibitors have been developed for oncology indications up to phase I/II clinical trials, but were stopped due to toxicity and efficacy issues.

Most of the developed inhibitors are nucleoside-analogue prodrugs (3-deazauridine, CPEC, carbodine), which are converted to the active triphosphorylated metabolite by the kinases involved in pyrimidine biosynthesis: uridine/cytidine kinase, nucleoside monophosphate-kinase (NMP-kinase) and nucleoside diphosphatekinase (NDP-kinase). The remaining inhibitors (acivicin, DON) are reactive analogues of glutamine, which irreversibly inhibit the glutaminase domain of CTPS. Gemcitibine is also reported to have some inhibitory activity against CTPS (McClusky et al., 2016).

CTPS therefore appears to be an important target in the cancer field. The nature of all of the above compounds is such that effects on other pathways are likely to contribute to the efficacy they show in inhibiting tumours.

Selective CTPS inhibitors therefore offer an attractive alternative approach for the treatment of tumours. Compounds with different potencies against CTPS1 and CTPS2 may offer important opportunities to target different tumours depending upon their relative dependence on these enzymes.

CTPS1 has also been suggested to play a role in vascular smooth muscle cell proliferation following vascular injury or surgery (Tang, et al. 2013).

As far as is known to date, no selective CTPS1 inhibitors have been developed. Recently, the CTPS1 selective inhibitory peptide CTpep-3 has been identified. The inhibitory effects of CTpep-3 however, were seen in cell free assays but not in the cellular context. This was not unexpected though, since the peptide is unlikely to enter the cell and hence is not easily developable as a therapeutic (Sakamoto, et al. 2017).

In summary, the available information and data strongly suggest that inhibitors of CTPS1 will reduce the proliferation of a number of immune and cancer cell populations, with the potential for an effect on other selected cell types such as vascular smooth muscle cells as well. Inhibitors of CTPS1 may therefore be expected to have utility for treatment or prophylaxis in a wide range of indications where the pathology is driven by these populations.

CTPS1 inhibitors represent a novel approach for inhibiting selected components of the immune system in various tissues, and the related pathologies or pathological conditions such as, in general terms, rejection of transplanted cells and tissues, Graft-related diseases or disorders, allergies and autoimmune diseases. In addition, CTPS1 inhibitors offer therapeutic potential in a range of cancer indications and in enhancing recovery from vascular injury or surgery and reducing morbidity and mortality associated with neointima and restenosis.

International patent applications VNO2019/106156, WO2019/106146, WO02019/179652 and WO2019/180244 and WO2020/083975 disclose CTPS1 inhibitors.

### SUMMARY OF THE INVENTION

The invention provides a compound of formula (I):

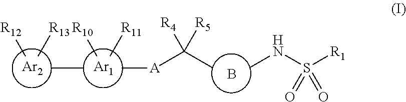

wherein ring B is selected from the group consisting of:

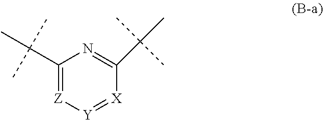

wherein X, Y and Z are as defined below; and

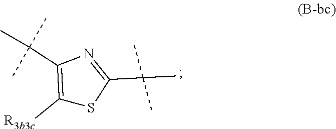

wherein R_{3b3c} is R_{3b} or R_{3c} as defined below;

wherein when B is (B-a) the compound of formula (I) is a compound of formula (I-a):

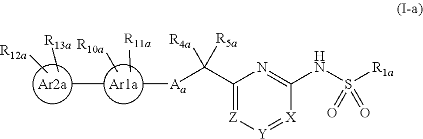

wherein:
Aₐ is Aₐₐ or A_{ba};
   wherein:
   Aₐₐ is an amine linker having the following structure: —NH—, —CH₂NH— or —NHCH₂—;
   A_{ba} is an amide linker having the following structure: —C(═O)NH— or —NHC(═O)—;
X is N or CH;
Y is N or CR₂ₐ;
Z is N or CR₃ₐ,
   with the proviso that when at least one of X or Z is N, Y cannot be N;
R₂ₐ is H, halo, C₁₋₂alkyl, OC₁₋₂alkyl, C₁₋₂haloalkyl or OC₁₋₂haloalkyl; and
R₃ₐ is H, halo, CH₃, OCH₃, CF₃ or OCF₃;
   wherein at least one of R₂ₐ and R₃ₐ is H;
R₁ₐ is R₁ₐₐ or R_{1ba};
   wherein:
   R₁ₐₐ is NR₃₂ₐR₃₃ₐ;
   R_{1ba} is C₁₋₅alkyl, C₀₋₂alkyleneC₃₋₅cycloalkyl which cycloalkyl is optionally substituted by CH₃, or CF₃;
R₄ₐ and R₅ₐ are R₄ₐₐ and R₅ₐₐ, or R_{4ba} and R_{5ba};
   wherein:
   R₄ₐₐ and R₅ₐₐ together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆ heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR₂₁ₐR₂₂ₐ; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐₐ and R₅ₐₐ together with the carbon atom to which they are attached may be substituted by one or two substituents, each substituent being independently selected from the group consisting of C₁₋₃ alkyl or OC₁₋₃alkyl; or
   R₄ₐₐ and R₅ₐₐ together with the carbon atom to which they are attached form a C₃₋₆heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl; or
   R₄ₐₐ and R₅ₐₐ together with the carbon atom to which they are attached form a C₃₋₆heterocycloalkyl comprising one nitrogen atom, wherein said nitrogen atom is substituted by —S(O)₂R₂₉ₐ; or
   R_{4ba} and R_{5ba} are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R_{4ba} and R_{5ba} together with the carbon atom to which they are attached form a C₃₋₆cycloalkyl or C₃₋₆heterocycloalkyl; and
   when Aₐ is —NHC(═O)— or —NHCH₂—:
   R_{4ba} and R_{5ba} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and NR₂₁ₐR₂₂ₐ;
Ar1a is a 6-membered aryl or heteroaryl;
Ar2a is a 6-membered aryl or heteroaryl and is attached to Ar1a in the para position relative to group Aₐ;
R₁₀ₐ is H, halo, C₁₋₃alkyl, C₁₋₂haloalkyl, OC₁₋₂alkyl, OC₁₋₂haloalkyl or CN;
R₁₁ₐ is H, F, Cl, C₁₋₂alkyl, CF₃, OCH₃ or CN;
R₁₂ₐ is attached to Ar2 in the ortho or meta position relative to Ar1a and R₁₂ₐ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alky)₂, NHC(O)C₁₋₃alkyl or NR₂₃ₐR₂₄ₐ; and
   when Aₐ is —NHC(═O)—, —NH— or —NHCH₂—:
   R₁₂ₐ may additionally be selected from CN, OCH₂CH₂N(CH₃)₂ and a C₃₋₆heterocycloalkyl comprising one nitrogen located at the point of attachment to Ar2a, or R₁₂ₐ together with a nitrogen atom to which it is attached forms an N-oxide (N⁺—O⁻);
R₁₃ₐ is H or halo;
R₂₁ₐ is H, C₁₋₅alkyl, C(O)C₁₋₅alkyl, C(O)OC₁₋₅alkyl, C₁₋₃alkylOC₁₋₂alkyl, C₁₋₄haloalkyl, or C₄₋₆heterocycloalkyl;
R₂₂ₐ is H or CH₃;
R₂₃ₐ is H or C₁₋₂alkyl; and
R₂₄ₐ is H or C₁₋₂alkyl
R₂₉ₐ is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl;
R₃₂ₐ is C₁₋₃alkyl and R₃₃ is C₁₋₃alkyl; or
R₃₂ₐ and R₃₃ₐ together with the nitrogen atom to which they are attached form a C₃₋₅heterocycloalkyl;
wherein:
R₁ₐ is R₁ₐₐ; and/or
R₄ₐ and R₅ₐ are R₄ₐₐ and R₅ₐₐ; and/or
Aₐ is Aₐₐ; and

wherein when B is (B-bc) and R_{3b3c} is R_{3b}, the compound of formula (I) is a compound of formula (I-b):

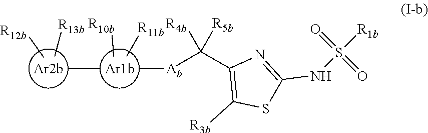

A_{b} is A_{ab} or A_{bb};
wherein:
   A_{ab} is —NR_{6b}CH₂— or —NR_{6b}—;
   A_{bb} is —NR_{6b}C(═O)—;
R_{1b} is R_{1ab} or R_{1bb};
wherein:
   R_{1ab} is NR_{32b}R_{33b};
   R_{1bb} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R_{3b} is H, halo, CH₃, OC₁₋₂alkyl or CF₃;
or R_{3b} together with R_{5bb} forms a 5- or 6-membered cycloalkyl or 5 or 6 membered oxygen-containing heterocycloalkyl;
R_{4b} and R_{5b} are either R_{4ab} and R_{5ab} or R_{4bb} and R_{5bb};
wherein:
   R_{4ab} and R_{5ab} together with the carbon atom to which they are attached form a C₃₋₆cycloalkyl which is:
      substituted by one or two substituents, each substituent being independently selected from the group consisting of C₁₋₃alkyl, oxo, OH, C₁₋₃ alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21b}R_{22b}; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ab} and R_{5ab} together with the carbon atom to which they are attached may be substituted by one or two substituents, each substituent being independently selected from the group consisting of C₁₋₃ alkyl or OC₁₋₃alkyl; or
   R_{4ab} and R_{5ab} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heteroycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl; or
   R_{4ab} and R_{5ab} together with the carbon atom to which they are attached form a C₃₋₆heterocycloalkyl comprising one nitrogen atom, wherein said nitrogen atom is substituted by —S(O)₂R_{29b}; or
   R_{4bb} and R_{5bb} are each independently H, halo, C₁₋₆alkyl, C₀₋₂alkyleneC₃₋₆cycloalkyl, C₀₋₂alkyleneC₃₋₆heterocycloalkyl, OC₁₋₆alkyl, OC₀₋₂alkyleneC₃₋₆cycloalkyl, C₁₋₃alkyleneOC₁₋₃alkyl, C₁₋₆alkylOH, C₁₋₆haloalkyl, OC₁₋₆haloalkyl or NR_{21b}R_{22b},
      or R_{4bb} is H and R_{5bb} together with R_{3b} form a 5- or 6-membered cycloalkyl or 5 or 6 membered oxygen-containing heterocycloalkyl,
      or R_{4bb} and R_{5bb} together with the carbon atom to which they are attached form a C₃₋₆cycloalkyl or C₃₋₆heterocycloalkyl,
      or R_{4bb} is H and R_{5bb} and R_{6b} are a C₂₋₃alkylene chain forming a 5- or 6-membered ring;
      or R_{4bb} is O and R_{5bb} is absent;
R_{6b} is H or C₁₋₃alkyl,
   or R_{6b} together with R_{11b} when in the ortho-position to group A_{b} are a C₂alkylene chain forming a 5-membered ring,
   or R_{5bb} and R_{6b} are a C₂₋₃alkylene chain forming a 5- or 6-membered ring and R_{4bb} is H;
Ar1b is 6-membered aryl or heteroaryl;
Ar2b is a 6-membered aryl or heteroaryl and is attached to Ar1b in the para position relative to group A_{b};
R_{10b} is H, halo, C₁₋₃alkyl, OC₁₋₂alkyl, C₁₋₂haloalkyl, OC₁₋₂haloalkyl or CN;
R_{11b} is H, F, Cl, CH₃, ethyl, OCH₃, CF₃, OCF₃ or CN,
   or R_{11b}, when in the ortho-position to group A_{b}, together with R_{6b} are a C₂alkylene chain forming a 5-membered ring;
R_{12b} is attached to Ar2b in the ortho or meta position relative to Ar1b and R_{12b} is H, halo, C₁₋₄alkyl, C₂₋₄alkynyl, C₀₋₂alkyleneC₃₋₅cycloalkyl, OC₁₋₄alkyl, OC₀₋₂alkyleneC₃₋₅cycloalkyl, OCH₂CH₂N(CH₃)₂, OH, C₁₋₄alkylOH, CN, C₁₋₃alkyleneOC₁₋₂alkyl, C₁₋₄haloalkyl, OC₁₋₄haloalkyl, C(═O)C₁₋₂alkyl, NR_{23b}R_{24b}, SO₂C₁₋₄alkyl, SOC₁₋₄alkyl, SC₁₋₄alkyl, SH, C(O)N(CH₃)₂, NHC(O)C₁₋₃alkyl, C₃₋₆heterocycloalkyl comprising one nitrogen located at the point of attachment to Ar2b, or R_{12b} together with a nitrogen atom to which it is attached forms an N-oxide (N⁺—O⁻);
R_{13b} is H, halo, CH₃ or OCH₃;
R_{21b} is H, C₁₋₅alkyl, C(O)C₁₋₅alkyl, C(O)OC₁₋₅alkyl, C₁₋₃alkylOC₁₋₂alkyl, C₁₋₄haloalkyl, or C₄₋₆heterocycloalkyl;
R_{22b} is H or CH₃;
R_{23b} is H or C₁₋₂alkyl;
R_{24b} is H or C₁₋₂alkyl;
R_{29b} is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl; and
R_{32b} is C₁₋₃alkyl and R_{33b} is C₁₋₃alkyl; or
R_{32b} and R_{33b} together with the nitrogen atom to which they are attached form a C₃₋₅heterocycloalkyl;
wherein:
R_{1b} is R_{1ab}; and/or
R_{4b} and R_{5b} are R_{4ab} and R_{5ab}; and/or
A is A_{b}; or

wherein when B is (B-bc) and R_{3b3c} is R_{3c}, the compound of formula (I) is a compound of formula (I-c):

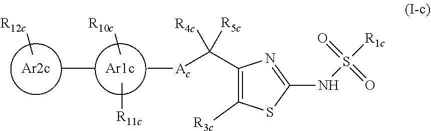

wherein:
A_{c} is A_{ac} or A_{bc};
   wherein:
   A_{ac} is —CH₂NR_{6c}—;
   A_{bc} is —C(═O)NR_{6c}—;
R_{1c} is R_{1ac} or R_{1bc};
wherein:
   R_{1ac} is NR_{32c}R_{33c};
R_{1bc} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R_{3c} is H, CH₃, halo, OC₁₋₂alkyl or CF₃;
R_{4c} and R_{5c} are either R_{4ac} and R_{5ac} or R_{4bc} and R_{5bc};
wherein:
   R_{4ac} and R_{5ac} together with the carbon atom to which they are attached form a C₃₋₆cycloalkyl which is:
      substituted by one or two substituents, each substituent being independently selected from the group consisting of C₁₋₃alkyl, oxo, OH, C₁₋₃ alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21c}R_{22c}; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ac} and R_{5ac} together with the carbon atom to which they are attached may be substituted by one or two substituents, each substituent being independently selected from the group consisting of C₁₋₃ alkyl or OC₁₋₃alkyl; or
   R_{4ac} and R_{5ac} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heteroycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl; or
   R_{4ac} and R_{5ac} together with the carbon atom to which they are attached form a C₃₋₆heterocycloalkyl comprising one nitrogen atom, wherein said nitrogen atom is substituted by —S(O)₂R_{29c}; or
   R_{4bc} and R_{5ac} are each independently H, C₁₋₃alkyl, C₀₋₂alkyleneC₃₋₆cycloalkyl, C₀₋₂alkyleneC₃₋₆heterocycloalkyl, C₁₋₃alkyleneOC₁₋₃alkyl, C₁₋₃alkylOH or C₁₋₆haloalkyl,
   or R_{4bc} and R_{5bc} together with the carbon atom to which they are attached form a C₃₋₆cycloalkyl or C₃₋₆heterocycloalkyl ring;
R_{6c} is H or C₁₋₃alkyl;
Ar1c is a 6-membered aryl or heteroaryl;
Ar2c is a 6-membered aryl or heteroaryl and is attached to Ar1c in the para position relative to group A_{c};
R_{10c} is H, halo, C₁₋₃alkyl, OC₁₋₂alkyl, C₁₋₂haloalkyl, OC₁₋₂haloalkyl or CN;
R_{11c} is H, F, Cl, CH₃, ethyl, OCH₃, CF₃, OCF₃ or CN;
R_{12c} is attached to Ar2c in the meta or ortho position relative to Ar1c and R_{12c} is H, halo, C₁₋₄alkyl, C₂₋₄alkynyl, C(═O)C₁₋₂alkyl, C₀₋₂alkyleneC₃₋₅cycloalkyl, OC₁₋₄alkyl, C₁₋₃ alkyleneOC₁₋₃alkyl, C₁₋₄haloalkyl, OC₁₋₄haloalkyl, CN, OC₀₋₂alkyleneC₃₋₅cycloalkyl, OCH₂CH₂N(CH₃)₂, OH, C₁₋₄alkylOH, NR_{23c}R_{24c}, SO₂CH₃, C(O)N(CH₃)₂, NHC(O)C₁₋₃alkyl, or a C₃₋₆heterocycloalkyl comprising one nitrogen located at the point of attachment to Ar2c, or R_{12c} together with a nitrogen atom to which it is attached forms an N-oxide (N⁺—O⁻);
R_{21c} is H, C₁₋₅alkyl, C(O)C₁₋₅alkyl, C(O)OC₁₋₅alkyl, C₁₋₃alkylOC₁₋₂alkyl, C₁₋₄haloalkyl, or C₄₋₆heterocycloalkyl;
R_{22c} is H or CH₃;
R_{23c} is H or C₁₋₂alkyl;
R_{24c} is H or C₁₋₂alkyl;
R_{29c} is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl; and
R_{32c} is C₁₋₃alkyl and R_{33c} is C₁₋₃alkyl; or
R_{32c} and R_{33c} together with the nitrogen atom to which they are attached form a C₃₋₅heterocycloalkyl;
   wherein:
R_{1c} is R_{1ac}; and/or
R_{4c} and R_{5c} are R_{4ac} and R_{5ac}; and/or
A_{c} is A_{ac}.

In one embodiment, the compound formula (I) is a compound of formula (I-a).

In another embodiment, the compound formula (I) is a compound of formula (I-b).

In another embodiment, the compound formula (I) is a compound of formula (I-c).

A compound of formula (I) may be provided in the form of a salt and/or solvate thereof and/or derivative thereof. Suitably, the compound of formula (I) may be provided in the form of a pharmaceutically acceptable salt and/or solvate thereof and/or derivative thereof. In particular, the compound of formula (I) may be provided in the form of a pharmaceutically acceptable salt and/or solvate, such as a pharmaceutically acceptable salt.

Also provided is a compound of formula (I), or a pharmaceutically acceptable salt and/or solvate thereof and/or derivative thereof, for use as a medicament, in particular for use in the inhibition of CTPS1 in a subject or the prophylaxis or treatment of associated diseases or disorders, such as those in which a reduction in T-cell and/or B-cell proliferation would be beneficial.

Further, there is provided a method for the inhibition of CTPS1 in a subject or the prophylaxis or treatment of associated diseases or disorders, such as those in which a reduction in T-cell and/or B-cell proliferation would be beneficial, by administering to a subject in need thereof a compound of formula (I) or a pharmaceutically acceptable salt and/or solvate thereof and/or derivative thereof.

Additionally provided is the use of a compound of formula (I), or a pharmaceutically acceptable salt and/or solvate thereof and/or derivative thereof, in the manufacture of a medicament for the inhibition of CTPS1 in a subject or the prophylaxis or treatment of associated diseases or disorders, such as those in which a reduction in T-cell and/or B-cell proliferation would be beneficial.

Suitably the disease or disorder is selected from: inflammatory skin diseases such as psoriasis or lichen planus; acute and/or chronic GVHD such as steroid resistant acute GVHD; acute lymphoproliferative syndrome (ALPS); systemic lupus erythematosus, lupus nephritis or cutaneous lupus; and transplantation. In addition, the disease or disorder may be selected from myasthenia gravis, multiple sclerosis, and scleroderma/systemic sclerosis.

Also provided is a compound of formula (I), or a pharmaceutically acceptable salt and/or solvate thereof and/or derivative thereof, for use in the treatment of cancer.

Further, there is provided a method for treating cancer in a subject, by administering to a subject in need thereof a compound of formula (I) or a pharmaceutically acceptable salt and/or solvate thereof and/or derivative thereof.

Additionally provided is the use of a compound of formula (I), or a pharmaceutically acceptable salt and/or solvate thereof and/or derivative thereof, in the manufacture of a medicament for the treatment of cancer in a subject.

Also provided is a compound of formula (I), or a pharmaceutically acceptable salt and/or solvate thereof and/or derivative thereof, for use in enhancing recovery from vascular injury or surgery and reducing morbidity and mortality associated with neointima and restenosis in a subject.

Further, there is provided a method for enhancing recovery from vascular injury or surgery and reducing morbidity and mortality associated with neointima and restenosis in a subject, by administering to a subject in need thereof a compound of formula (I) or a pharmaceutically acceptable salt and/or solvate thereof and/or derivative thereof.

Additionally provided is the use of a compound of formula (I), or a pharmaceutically acceptable salt and/or solvate thereof and/or derivative thereof, in the manufacture of a medicament for enhancing recovery from vascular injury or surgery and reducing morbidity and mortality associated with neointima and restenosis in a subject.

Also provided are pharmaceutical compositions containing a compound of formula (I), or a pharmaceutically acceptable salt and/or solvate thereof and/or derivative thereof, and a pharmaceutically acceptable carrier or excipient.

Also provided are processes for preparing compounds of formula (I) and novel intermediates of use in the preparation of compounds of formula (I).

### DETAILED DESCRIPTION OF THE INVENTION

In one embodiment there is provided a compound of formula (I) as described above, or a salt such as a pharmaceutically acceptable and/or solvate and/or derivative thereof.

Suitably, the invention provides a compound of formula (I):

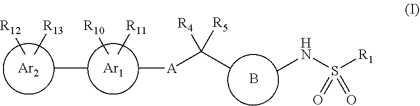

wherein ring B is selected from the group consisting of:

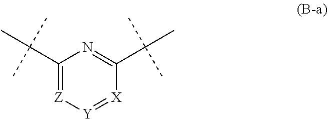

wherein X, Y and Z are as defined below; and

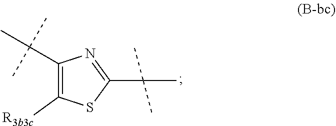

wherein R_{3b3c} is R_{3b} or R_{3c} as defined below;

wherein when B is (B-a) the compound of formula (I) is a compound of formula (I-a):

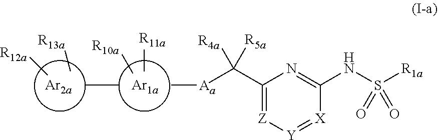

wherein:
Aₐ is Aₐₐ or A_{ba};
   wherein:
   Aₐₐ is an amine linker having the following structure: —NH—, —CH₂NH— or —NHCH₂—;
   A_{ba} is an amide linker having the following structure: —C(═O)NH— or —NHC(═O)—;
X is N or CH;
Y is N or CR₂ₐ;
Z is N or CR₃ₐ;
   with the proviso that when at least one of X or Z is N, Y cannot be N;
R₂ₐ is H, halo, C₁₋₂alkyl, OC₁₋₂alkyl, C₁₋₂haloalkyl or OC₁₋₂haloalkyl; and
R₃ₐ is H, halo, CH₃, OCH₃, CF₃ or OCF₃;
   wherein at least one of R₂ₐ and R₃ₐ is H;
R₁ₐ is R₁ₐₐ or R_{1ba};
   wherein:
   R₁ₐₐ is NR₃₂ₐR₃₃ₐ;
   R_{1ba} is C₁₋₅alkyl, C₀₋₂alkyleneC₃₋₅cycloalkyl which cycloalkyl is optionally substituted by CH₃, or CF₃;
R₄ₐ and R₅ₐ are R₄ₐₐ and R₅ₐₐ or R_{4ba} and R_{5ba};
   wherein:
   R₄ₐ and R₅ₐ together with the carbon atom to which they are attached form a C₃₋₆cycloalkyl which is:
      substituted by one or two substituents, each substituent being independently selected from the group consisting of C₁₋₃alkyl, oxo, OH, C₁₋₃ alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR₂₁ₐR₂₂ₐ; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐₐ and R₅ₐₐ together with the carbon atom to which they are attached may be substituted by one or two substituents, each substituent being independently selected from the group consisting of C₁₋₃ alkyl or OC₁₋₃alkyl; or
   R₄ₐₐ and R₅ₐₐ together with the carbon atom to which they are attached form a C₃₋₆heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl; or
   R₄ₐₐ and R₅ₐₐ together with the carbon atom to which they are attached form a C₃₋₆heterocycloalkyl comprising one nitrogen atom, wherein said nitrogen atom is substituted by —S(O)₂R₂₉ₐ; or
   R_{4ba} and R_{5ba} are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R_{4ba} and R_{5ba} together with the carbon atom to which they are attached form a C₃₋₆ cycloalkyl or C₃₋₆heterocycloalkyl; and
   when Aₐ is —NHC(═O)— or —NHCH₂—:
   R_{4ba} and R_{5ba} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and NR₂₁ₐR₂₂ₐ;
Ar1a is a 6-membered aryl or heteroaryl;
Ar2a is a 6-membered aryl or heteroaryl and is attached to Ar1a in the para position relative to group Aₐ;
R₁₀ₐ is H, halo, C₁₋₃alkyl, C₁₋₂haloalkyl, OC₁₋₂alkyl, OC₁₋₂haloalkyl or CN;
R₁₁ₐ is H, F, Cl, C₁₋₂alkyl, CF₃, OCH₃ or CN;
R₁₂ₐ is attached to Ar2 in the ortho or meta position relative to Ar1a and R₁₂ₐ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃ₐR₂₄ₐ; and
   when Aₐ is —NHC(═O)—, —NH— or —NHCH₂—:
   R₁₂ₐ may additionally be selected from CN, OCH₂CH₂N(CH₃)₂ and a C₃₋₆heterocycloalkyl comprising one nitrogen located at the point of attachment to Ar2a, or R₁₂ₐ together with a nitrogen atom to which it is attached forms an N-oxide (N⁺—O⁻);
R₁₃ₐ is H or halo;
R₂₁ₐ is H, C₁₋₅alkyl, C(O)C₁₋₅alkyl, C(O)OC₁₋₅alkyl;
R₂₂ₐ is H or CH₃;
R₂₃ₐ is H or C₁₋₂alkyl; and
R₂₄ₐ is H or C₁₋₂alkyl
R₂₉ₐ is C₁₋₃alkyl, C₀₋₂alkyleneC₃₋₅cycloalkyl which cycloalkyl is optionally substituted by CH₃, or CF₃;
R₃₂ₐ is C₁₋₃alkyl and R₃₃ is C₁₋₃alkyl; or
R₃₂ₐ and R₃₃ₐ together with the nitrogen atom to which they are attached form a C₃₋₅heterocycloalkyl;

wherein
R₁ₐ is R₁ₐₐ; and/or
R₄ₐ and R₅ₐ are R₄ₐₐ and R₅ₐₐ; and/or
Aₐ is Aₐₐ; and
   wherein when B is (B-bc) and R_{3b3c} is R_{3b}, the compound of formula (I) is a compound of formula (I-b):

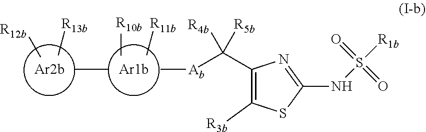

wherein:
A_{b} is A_{ab} or A_{bb};
   wherein:
      A_{ab} is —NR_{6b}CH₂— or —NR_{6b}—;
      A_{bb} is —NR_{6b}C(═O)—;
R_{1b} is R_{1ab} or R_{1bb};
wherein:
   R_{1ab} is NR_{32b}R_{33b};
   R_{1bb} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R_{3b} is H, halo, CH₃, OC₁₋₂alkyl or CF₃;
or R_{3b} together with R_{5b} forms a 5- or 6-membered cycloalkyl or 5 or 6 membered oxygen-containing heterocycloalkyl;
R_{4b} and R_{5b} are either R_{4ab} and R_{5ab} or R_{4bb} and R_{5bb};
wherein:
   R_{4ab} and R_{5ab} together with the carbon atom to which they are attached form a C₃₋₆cycloalkyl which is:
      substituted by one or two substituents, each substituent being independently selected from the group consisting of C₁₋₃alkyl, oxo, OH, C₁₋₃ alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21b}R_{22b}; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ab} and R_{5ab} together with the carbon atom to which they are attached may be substituted by one or two substituents, each substituent being independently selected from the group consisting of C₁₋₃ alkyl or OC₁₋₃alkyl; or
   R_{4ab} and R_{5ab} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heteroycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl; or
   R_{4ab} and R_{5ab} together with the carbon atom to which they are attached form a C₃₋₆heterocycloalkyl comprising one nitrogen atom, wherein said nitrogen atom is substituted by —S(O)₂R_{29b}; or
   R_{4bb} and R_{5bb} are each independently H, halo, C₁₋₆alkyl, C₀₋₂alkyleneC₃₋₆cycloalkyl, C₀₋₂alkyleneC₃₋₆heterocycloalkyl, OC₁₋₆alkyl, OC₀₋₂alkyleneC₃₋₄cycloalkyl, C₁₋₃alkyleneOC₁₋₃alkyl, C₁₋₆alkylOH, C₁₋₆haloalkyl, OC₁₋₆haloalkyl or NR_{21b}R_{22b},
      or R_{4bb} is H and R_{5bb} together with R_{3b} form a 5- or 6-membered cycloalkyl or 5 or 6 membered oxygen-containing heterocycloalkyl,
      or R_{4bb} and R_{5bb} together with the carbon atom to which they are attached form a C₃₋₆cycloalkyl or C₃₋₆heterocycloalkyl,
      or R_{4bb} is H and R_{5bb} and R_{6b} are a C₂₋₃alkylene chain forming a 5- or 6-membered ring;
      or R_{4bb} is O and R_{5bb} is absent;
R_{6b} is H or C₁₋₃alkyl,
   or R_{6b} together with R_{11b} when in the ortho-position to group A_{b} are a C₂alkylene chain forming a 5-membered ring,
   or R_{5bb} and R_{6b} are a C₂₋₃alkylene chain forming a 5- or 6-membered ring and R_{4bb} is H;
Ar1b is 6-membered aryl or heteroaryl;
Ar2b is a 6-membered aryl or heteroaryl and is attached to Ar1b in the para position relative to group A_{b};
R_{10b} is H, halo, C₁₋₃alkyl, OC₁₋₂alkyl, C₁₋₂haloalkyl, OC₁₋₂haloalkyl or CN;
R_{11b} is H, F, Cl, CH₃, ethyl, OCH₃, CF₃, OCF₃ or CN,
   or R_{11b} when in the ortho-position to group A_{b}, together with R_{6b} are a C₂alkylene chain forming a 5-membered ring;
R_{12b} is attached to Ar2b in the ortho or meta position relative to Ar1b and R_{12b} is H, halo, C₁₋₄alkyl, C₂₋₄alkynyl, C₀₋₂alkyleneC₃₋₅cycloalkyl, OC₁₋₄alkyl, OC₀₋₂alkyleneC₃₋₅cycloalkyl, OCH₂CH₂N(CH₃)₂, OH, C₁₋₄alkylOH, CN, C₁₋₃alkyleneOC₁₋₃alkyl, C₁₋₄haloalkyl, OC₁₋₄haloalkyl, C(═O)C₁₋₂alkyl, NR_{23b}R_{24b}, SO₂C₁₋₄alkyl, SOC₁₋₄alkyl, SC₁₋₄alkyl, SH, C(O)N(CH₃)₂, NHC(O)C₁₋₃alkyl, C₃₋₆heterocycloalkyl comprising one nitrogen located at the point of attachment to Ar2b, or R_{12b} together with a nitrogen atom to which it is attached forms an N-oxide (N⁺—O⁻);
R_{13b} is H, halo, CH₃ or OCH₃;
R_{21b} is H, C₁₋₅alkyl, C(O)C₁₋₅alkyl, C(O)OC₁₋₅alkyl;
R_{22b} is H or CH₃;
R_{23b} is H or C₁₋₂alkyl;
R_{24b} is H or C₁₋₂alkyl;
R_{29b} is C₁₋₃alkyl, C₀₋₂alkyleneC₃₋₅cycloalkyl which cycloalkyl is optionally substituted by CH₃, or CF₃; and
R₃₂ₐ is C₁₋₃alkyl and R_{33b} is C₁₋₃alky; or
R_{32b} and R_{33b} together with the nitrogen atom to which they are attached form a C₃₋₅heterocycloalkyl;

wherein:
R_{1b} is R_{1ab}; and/or
R_{4b} and R_{5b} are R_{4ab} and R_{5ab}; and/or
A is A_{ab}; or

wherein when B is (B-bc) and R_{3b3c} is R_{3c}, the compound of formula (I) is a compound of formula (I-c):

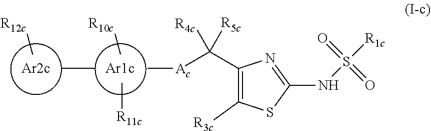

wherein:
A_{c} is A_{ac} or A_{bc};
   A_{ac} is —CH₂NR_{6c}—;
   A_{bc} is —C(═O)NR_{6c}—;
   R_{1c} is R_{1ac} or R_{1bc};
wherein:
   R_{1ac} is NR_{32c}R_{33c};
R_{1bc} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R_{3c} is H, CH₃, halo, OC₁₋₂alkyl or CF₃;
R_{4c} and R_{5c} are either R_{4ac} and R_{5ac} or R_{4bc} and R_{5bc};
wherein:
   R_{4ac} and R_{5ac} together with the carbon atom to which they are attached form a C₃₋₆cycloalkyl which is:
      substituted by one or two substituents, each substituent being independently selected from the group consisting of C₁₋₃alkyl, oxo, OH, C₁₋₃ alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₄heterocycloalkyl, OC₁₋₃alkyl and NR_{21c}R_{22c}; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ac} and R_{5ac} together with the carbon atom to which they are attached may be substituted by one or two substituents, each substituent being independently selected from the group consisting of C₁₋₃ alkyl or OC₁₋₃alkyl; or
   R_{4ac} and R_{5ac} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heteroycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl; or
   R_{4ac} and R_{5ac} together with the carbon atom to which they are attached form a C₃₋₆heterocycloalkyl comprising one nitrogen atom, wherein said nitrogen atom is substituted by —S(O)₂R_{29c}; or
   R_{4bc} and R_{5bc} are each independently H, C₁₋₆alkyl, C₀₋₂alkyleneC₃₋₆cycloalkyl, C₀₋₂alkyleneC₃₋₆heterocycloalkyl, C₁₋₃alkyleneOC₁₋₃alkyl, C₁₋₆alkylOH or C₁₋₆haloalkyl,
   or R_{4bc} and R_{5bc} together with the carbon atom to which they are attached form a C₃₋₆cycloalkyl or C₃₋₆heterocycloalkyl ring;
R_{6c} is H or C₁₋₃alkyl;
Ar1c is a 6-membered aryl or heteroaryl;
Ar2c is a 6-membered aryl or heteroaryl and is attached to Ar1c in the pare position relative to group A_{c};
R_{10c} is H, halo, C₁₋₃alkyl, OC₁₋₂alkyl, C₁₋₂haloalkyl, OC₁₋₂haloalkyl or CN;
R_{11c} is H, F, Cl, CH₃, ethyl, OCH₃, CF₃, OCF₃ or CN;
R_{12c} is attached to Ar2c in the meta or ortho position relative to Ar1c and R_{12c} is H, halo, C₁₋₄alkyl, C₂₋₄alkynyl, C(═O)C₁₋₂alkyl, C₀₋₂alkyleneC₃₋₅cycloalkyl, OC₁₋₄alkyl, C₁₋₃alkyleneOC₁₋₃alkyl, C₁₋₄haloalkyl, OC₁₋₄haloalkyl, CN, OC₀₋₂alkyleneC₃₋₅cycloalkyl, OCH₂CH₂N(CH₃)₂, OH, C₁₋₄alkylOH, NR_{23c}R_{24c}, SO₂CH₃, C(O)N(CH₃)₂, NHC(O)C₁₋₃alkyl, or a C₃₋₆heterocycloalkyl comprising one nitrogen located at the point of attachment to Ar2c, or R_{12c} together with a nitrogen atom to which it is attached forms an N-oxide (N⁺—O⁻);
R_{21c} is H, C₁₋₅alkyl, C(O)C₁₋₅alkyl, C(O)OC₁₋₅alkyl;
R_{22c} is H or CH₃;
R_{23c} is H or C₁₋₂alkyl;
R_{24c} is H or C₁₋₂alkyl;
R_{29c} is C₁₋₃ alkyl, C₀₋₂alkyleneC₃₋₅cycloalkyl which cycloalkyl is optionally substituted by CH₃, or CF₃; and
R_{32c} is C₁₋₃alkyl and R_{33c} is C₁₋₃alkyl; or
R_{32c} and R_{33c} together with the nitrogen atom to which they are attached form a C₃₋₅heterocycloalkyl;
   wherein:
R_{1c} is R_{1ac}; and/or
R_{4c} and R_{5c} are R_{4ac} and R_{5ac}; and/or
A_{c} is A_{ac}.

More suitably, there is provided a compound of formula (I-a).

Alternatively, there is provided a compound of formula (I-b).

Alternatively, there is provided a compound of formula (I-c).

Compounds of Formula (I-a)

The invention provides a compound of formula (I-a):

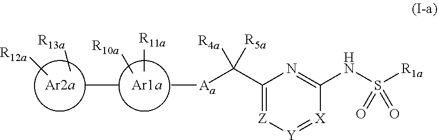

wherein:
Aₐ is Aₐₐ or A_{ba};
wherein:
   Aₐₐ is an amine linker having the following structure: —NH—, —CH₂NH— or —NHCH₂—;
   A_{ba} is an amide linker having the following structure: —C(═O)NH— or —NHC(═O)—;
X is N or CH;
Y is N or CR₂ₐ;
Z is N or CR₃ₐ;
   with the proviso that when at least one of X or Z is N, Y cannot be N;
R₁ₐ is R₁ₐₐ or R_{1ba};
   wherein:
   R₁ₐₐ is NR₃₂ₐR₃₃ₐ;
   R_{1ba} is C₁₋₅alkyl, C₀₋₂alkyleneC₃₋₅cycloalkyl which cycloalkyl is optionally substituted by CH₃, or CF₃;
R₂ₐ is H, halo, C₁₋₂alkyl, OC₁₋₂alkyl, C₁₋₂haloalkyl or OC₁₋₂haloalkyl;
R₃ₐ is H, halo, CH₃, OCH₃, CF₃ or OCF₃;
   wherein at least one of R₂ₐ and R₃ₐ is H;
R₄ₐ and R₅ₐ are R₄ₐₐ and R₅ₐₐ, or R_{4ba} and R_{5ba};
   wherein:
   R₄ₐₐ and R₅ₐₐ together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₄cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₄heterocycloalkyl, OC₁₋₃alkyl and NR₂₁ₐR₂₂ₐ; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐₐ and R₅ₐₐ together with the carbon atom to which they are attached may be substituted by one or two substituents, each substituent being independently selected from the group consisting of C₁₋₃ alkyl or OC₁₋₃alkyl; or
      R₄ₐₐ and R₅ₐₐ together with the carbon atom to which they are attached form a C₃₋₆heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl; or
      R₄ₐₐ and R₅ₐₐ together with the carbon atom to which they are attached form a C₃₋₆heterocycloalkyl comprising one nitrogen atom, wherein said nitrogen atom is substituted by —S(O)₂R₂₉ₐ; or
      R_{4ba} and R_{5ba} are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R_{4ba} and R_{5ba} together with the carbon atom to which they are attached form a C₃₋₆cycloalkyl or C₃₋₆heterocycloalkyl; and
      when Aₐ is —NHC(═O)— or —NHCH₂—:
      R_{4ba} and R_{5ba} may additionally be selected from halo, OC₁₋₆haloalkyl, OC₀₋₂alkyleneC₃₋₆cycloalkyl, OC₀₋₂alkyleneC₃₋₄heterocycloalkyl, OC₁₋₆alkyl and NR₂₁ₐR₂₂ₐ;
Ar1a is a 6-membered aryl or heteroaryl;
Ar2a is a 6-membered aryl or heteroaryl and is attached to Ar1a in the para position relative to group Aₐ;
R₁₀ₐ is H, halo, C₁₋₃alkyl, C₁₋₂haloalkyl, OC₁₋₂alkyl, OC₁₋₂haloalkyl or CN;
R₁₁ₐ is H, F, Cl, C₁₋₂alkyl, CF₃, OCH₃ or CN;
R₁₂ₐ is attached to Ar2a in the ortho or meta position relative to Ar1a and R₁₂ₐ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃ₐR₂₄ₐ; and
   when Aₐ is —NHC(═O)—, —NH— or —NHCH₂—:
   R₁₂ₐ may additionally be selected from CN, OCH₂CH₂N(CH₃)₂ and a C₃₋₆heterocycloalkyl comprising one nitrogen located at the point of attachment to Ar2a, or R₁₂ₐ together with a nitrogen atom to which it is attached forms an N-oxide (N⁺—O⁻);
R₁₃ₐ is H or halo;
R₂₁ₐ is H, C₁₋₅alkyl, C(O)C₁₋₅alkyl, C(O)OC₁₋₅alkyl, C₁₋₅alkylOC₁₋₂alkyl, C₁₋₄haloalkyl, or C₄₋₆heterocycloalkyl;
R₂₂ₐ is H or CH₃;
R₂₃ₐ is H or C₁₋₂alkyl; and
R₂₄ₐ is H or C₁₋₂alkyl;
R₂₉ₐ is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl;
R₃₂ₐ is C₁₋₃alkyl and R₃₃ₐ is C₁₋₃alkyl; or
R₃₂ₐ and R₃₃ₐ together with the nitrogen atom to which they are attached form a C₃₋₅ heterocycloalkyl;

or a salt and/or solvate thereof and/or derivative thereof.

Suitably, R_{1c} is R_{1ac}; and/or R_{4c} and R_{5c} are R_{4ac} and R_{5ac}; and/or A_{c} is A_{ac}.

The invention also provides a compound of formula (I-a):

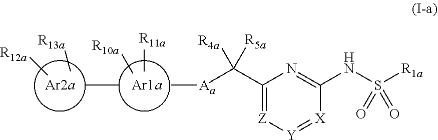

wherein:
Aₐ is Aₐₐ or A_{ba};
wherein:
   Aₐₐ is an amine linker having the following structure: —NH—, —CH₂NH— or —NHCH₂—;
   A_{ba} is an amide linker having the following structure: —C(═O)NH— or —NHC(═O)—;
X is N or CH;
Y is N or CR₂ₐ;
Z is N or CR₃ₐ;
   with the proviso that when at least one of X or Z is N, Y cannot be N;
R₁ₐ is R₁ₐₐ or R_{1ba};
   wherein:
   R₁ₐₐ is NR₃₂ₐR₃₃ₐ;
   R_{1ba} is C₁₋₅alkyl, C₀₋₂alkyleneC₃₋₅cycloalkyl which cycloalkyl is optionally substituted by CH₃, or CF₃;
R₂ₐ is H, halo, C₁₋₂alkyl, OC₁₋₂alkyl, C₁₋₂haloalkyl or OC₁₋₂haloalkyl;
R₃ₐ is H, halo, CH₃, OCH₃, CF₃ or OCF₃;
   wherein at least one of R₂ₐ and R₃ₐ is H;
R₄ₐ and R₅ₐ are R₄ₐₐ and R₅ₐₐ, or R_{4ba} and R_{5ba};
   wherein:
   R₄ₐₐ and R₅ₐₐ together with the carbon atom to which they are attached form a C₃₋₆cycloalkyl which is:
      substituted by one or two substituents, each substituent being independently selected from the group consisting of C₁₋₃alkyl, oxo, OH, C₁₋₃ alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR₂₁ₐR₂₂ₐ; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐₐ and R₅ₐₐ together with the carbon atom to which they are attached may be substituted by one or two substituents, each substituent being independently selected from the group consisting of C₁₋₃ alkyl or OC₁₋₃alkyl; or
R₄ₐₐ and R₅ₐₐ together with the carbon atom to which they are attached form a C₃₋₆ heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆ heterocycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl; or
   R₄ₐₐ and R₅ₐₐ together with the carbon atom to which they are attached form a C₃₋₆heterocycloalkyl comprising one nitrogen atom, wherein said nitrogen atom is substituted by —S(O)₂R₂₉ₐ; or
   R_{4ba} and R_{5ba}, are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R_{4ba} and R_{5ba} together with the carbon atom to which they are attached form a C₃₋₆cycloalkyl or C₃₋₆heterocycloalkyl; and
   when Aₐ is —NHC(═O)— or —NHCH₂—:
   R_{4ba} and R_{5ba} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and NR₂₁ₐR₂₂ₐ;
Ar1a is a 6-membered aryl or heteroaryl;
Ar2a is a 6-membered aryl or heteroaryl and is attached to Ar1a in the para position relative to group Aₐ;
R₁₀ₐ is H, halo, C₁₋₃alkyl, C₁₋₂haloalkyl, OC₁₋₂alkyl, OC₁₋₂haloalkyl or CN; R₁₁ₐ is H, F, Cl, C₁₋₂alkyl, CF₃, OCH₃ or CN; R₁₂ₐ is attached to Ar2a in the ortho or meta position relative to Ar1a and R₁₂ₐ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃ₐR₂₄ₐ; and
   when Aₐ is —NHC(═O)—, —NH— or —NHCH₂—:
   R₁₂ₐ may additionally be selected from CN, OCH₂CH₂N(CH₃)₂ and a C₃₋₆heterocycloalkyl comprising one nitrogen located at the point of attachment to Ar2a, or R₁₂ₐ together with a nitrogen atom to which it is attached forms an N-oxide (N⁺—O⁻);
R₁₃ₐ is H or halo;
R₂₁ₐ is H, C₁₋₅alkyl, C(O)C₁₋₅alkyl, C(O)OC₁₋₅alkyl;
R₂₁ₐ is H or CH₃;
R₂₃ₐ is H or C₁₋₂alkyl; and
R₂₄ₐ is H or C₁₋₂alky;
R₂₉ₐ is C₁₋₃alkyl, C₀₋₂alkyleneC₃₋₅cycloalkyl which cycloalkyl is optionally substituted by CH₃, or CF₃;
R₃₂ₐ is C₁₋₃alkyl and R₃₃ₐ is C₁₋₃alkyl; or
R₃₂ₐ and R₃₃ₐ together with the nitrogen atom to which they are attached form a C₃₋₅heterocycloalkyl;

or a salt and/or solvate thereof and/or derivative thereof.

Suitably, R_{1c} is R_{1ac}; and/or R_{4c} and R_{5c} are R_{4ac} and R_{5ac}; and/or A_{c} is A_{ac}.

The phrase ‘A_{ba} is an amide linker having the following structure: —C(═O)NH— or —NHC(═O)—’ means the following structures form:

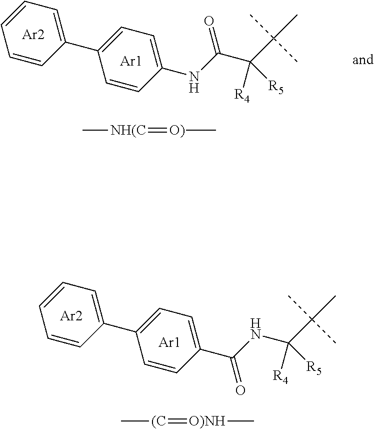

Reference to R₄ and R₅ above includes variables R₄ₐ, R₅ₐ, R₄ₐₐ, R₅ₐₐ, R_{4ba} and R_{5ba}, and reference to Ar1 and Ar2 includes variables Ar1a and Ar2a.

The phrase ‘Aₐₐ is an amine linker having the following structure: —CH₂NH— or —NHCH₂—’ means the following structures form:

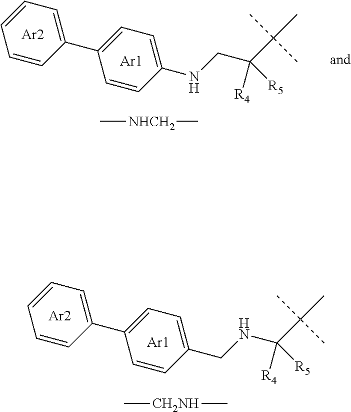

Reference to R₄ and R₅ above includes variables R₄ₐ, R₅ₐ, R₄ₐₐ, R₅ₐₐ, R_{4ba} and R_{5ba}, and reference to Ar1 and Ar2 includes variables Ar1a and Ar2a.

In one embodiment, A_{ba} is —C(═O)NH—. In another embodiment, A_{ba} is —NHC(═O)—. In an additional embodiment, Aₐₐ is —NH—. In a further embodiment, Aₐₐ is —CH₂NH—. In another embodiment, Aₐₐ is —NHCH₂—.

In one embodiment X is N. In another embodiment, X is CH.

In one embodiment, Y is N. In another embodiment, Y is CR₂ₐ.

In one embodiment. Z is N. In another embodiment, Z is CR₃ₐ.

Suitably, X is N, Y is CR₂ₐ and Z is CR₃ₐ. Alternatively, X is CH, Y is N and Z is CR₃ₐ. Alternatively, X is CH, Y is CR₂ₐ and Z is CR₃ₐ. Alternatively, X is CH, Y is CR₂ₐ and Z is N. Alternatively, X is N, Y is CR₂ₐ and Z is N.

In one embodiment of the invention, R₁ₐ is R₁ₐₐ, i.e. is NR₃₂ₐR₃₃ₐ. In an embodiment R₃₂, is C₁₋₃alkyl, such as methyl or ethyl, e.g. methyl. In an embodiment, R₃₃ₐ is C₁₋₃alkyl, such as methyl or ethyl, e.g. methyl. Suitably, R₃₂ₐ and R₃₃ₐ are both methyl. Suitably, R₃₂ₐ and R₃₃ₐ are both ethyl. Suitably, R₃₂ₐ is methyl and R₃₃ₐ is ethyl.

In another embodiment, R₃₂ₐ and R₃₃ₐ together with the nitrogen atom to which they are attached form a C₃₋₅heterocycloalkyl. Suitably, the C₃₋₅heterocycloalkyl is aziridinyl, azetidinyl or pyrrolidinyl.

Suitably, R₁ₐ is R_{1ba}.

In one embodiment of the invention R_{1ba} is C₁₋₅alkyl. When R_{1ba} is C₁₋₅alkyl, R_{1ba} may be methyl, ethyl, propyl (n-propyl or isopropyl), butyl (n-butyl, isobutyl, sec-butyl or tert-butyl) or pentyl (e.g. n-pentyl, sec-pentyl or 3-pentyl).

In a second embodiment of the invention R_{1ba} is C₀₋₂alkyleneC₃₋₅cycloalkyl which cycloalkyl is optionally substituted by CH₃. In some embodiments, R_{1ba} is C₀₋₂alkyleneC₃₋₅cycloalkyl. In other embodiments, R_{1ba} is C₀₋₂alkyleneC₃₋₅cycloalkyl which cycloalkyl is substituted by CH₃. R_{1ba} may be C₃₋₅cycloalkyl, which cycloalkyl is optionally substituted by CH₃. R_{1ba} may be C₁alkyleneC₃₋₅ cycloalkyl, which cycloalkyl is optionally substituted by CH₃. R_{1ba} may be C₂alkyleneC₃₋₅ cycloalkyl, which cycloalkyl is optionally substituted by CH₃. R_{1ba} may be C₀₋₂alkyleneC₃cycloalkyl, which cycloalkyl is optionally substituted by CH₃. R_{1ba} may be C₀₋₂alkyleneC₄cycloalkyl, which cycloalkyl is optionally substituted by CH₃. R_{1ba} may be C₀₋₂alkyleneC₅cycloalkyl, which cycloalkyl is optionally substituted by CH₃. Suitably, where C₀₋₂alkyleneC₃₋₅cycloalkyl is optionally substituted by CH₃, the CH₃ is at the point of attachment of the C₃₋₅cycloalkyl to the C₀₋₂alkylene.

In a third embodiment, R_{1ba} is CF₃.

Suitably R_{1ba} is cyclopropyl, cyclopropyl substituted by CH₃ at the point of attachment, cyclobutyl, CH₃ or CH₂CH₃. In particular R_{1ba} is cyclopropyl, cyclobutyl, CH₃ or CH₂CH₃, especially cyclopropyl.

In one embodiment, R₂ₐ is H. In a second embodiment, R₂ₐ is halo such as F, Cl or Br, e.g. Cl or Br. In a third embodiment, R₂ₐ is C₁₋₂alkyl. When R₂ₐ is C₁₋₂alkyl, R₂ₐ may be methyl or ethyl, such as methyl. In a fourth embodiment, R₂ₐ is OC₁₋₂alkyl. When R₂ₐ is OC₁₋₂alkyl, may be OCH₃ or OEt, such as OCH₃. In a fifth embodiment, R₂ₐ is C₁₋₂haloalkyl. When R₂ is C₁₋₂haloalkyl, R₂ₐ may be CF₃ or CH₂CF₃, such as CF₃. In a sixth embodiment, R₂ₐ is OC₁₋₂haloalkyl. When R₂ is OC₁₋₂ haloalkyl, R₂ₐ may be OCF₃ or OCH₂CF₃, such as OCF₃.

Suitably, R2 is H, CH₃ or CF₃, such as H or CH₃, in particular H.

In one embodiment R₃ₐ is H. In a second embodiment R₃ₐ is halo, in particular chloro or fluoro, especially fluoro. In a third embodiment, R₃ₐ is CH₃. In a fourth embodiment, R₃ₐ is OCH₃. In a fifth embodiment, R₃ₐ is CF₃. In a sixth embodiment, R₃ₐ is OCF₃.

Suitably, R₃ₐ is H, halo in particular chloro or fluoro, especially fluoro, CH₃ or CF₃. More suitably, R₃ₐ is H or F, such as H.

Suitably, at least one of R₂ₐ and R₃ₐ is H.

In one embodiment, R₄ₐ and R₅ₐ are R₄ₐₐ and R₅ₐₐ.

Suitably, R₄ₐₐ and R₅ₐₐ together with the carbon atom to which they are attached form a C₃₋₆cycloalkyl which is substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃ haloalkyl, OC₀₋₂alkyleneC₃₋₆cycloalkyl, OC₀₋₂alkyleneC₃₋₆heterocycloalkyl, OC₁₋₃alkyl and NR₂₁ₐR₂₂ₐ.

In one embodiment, the C₃₋₆cycloalkyl is cyclopropyl. In another embodiment, the C₃₋₆cycloalkyl is cyclobutyl. In another embodiment, the C₃₋₆cycloalkyl is cyclopentyl. In another embodiment, the C₃₋₆cycloalkyl is cyclohexyl.

In one embodiment the C₃₋₆cycloalkyl is substituted by one substituent. In a second embodiment the C₃₋₆cycloalkyl is substituted by two substituents.

In one embodiment, the substituent is C₁₋₃alkyl. Suitably, the substituent is methyl. Suitably, the substituent is ethyl. Suitably, the substituent is n-propyl. Suitably, the substituent is iso-propyl.

In a second embodiment, the substituent is C₁₋₃alkylOH. Suitably, the substituent is CH₂OH.

Suitably, the substituent is CH₂CH₂OH. Suitably, the substituent is CH₂CH₂CH₂OH.

In a third embodiment, the substituent is C₁₋₃haloalkyl. Suitably the C₁₋₃alkyl group is substituted by one, two or three, such as one, halogen atom. Suitably, the halogen atom is fluoro or chloro such as fluoro. Suitably, the substituent is C₁haloalkyl such as CF₃. Suitably, the substituent is C₂haloalkyl such as CH₂CF₃.

In a fourth embodiment, the substituent is C₀₋₂alkyleneC₃₋₆cycloalkyl, in particular C₀₋₂alkyleneC₃₋₅cycloalkyl, such as C₃₋₅cycloalkyl, C₁alkyleneC₃₋₅cycloalkyl or C₀₋₂alkyleneC₃₋₅cycloalkyl.

In a fifth embodiment, the substituent is C₀₋₂alkyleneC₃₋₆heterocycloalkyl such as C₀₋₂alkyleneC₃heterocycloalkyl, C₀₋₂alkyleneC₄heterocycloalkyl, C₀₋₂alkyleneC₅heterocycloalkyl, C₀₋₂alkyleneC₆heterocycloalkyl, C₀alkyleneC₃₋₆heterocycloalkyl, C₁alkyleneC₃₋₆heterocycloalkyl and C₂alkyleneC₃₋₆heterocycloalkyl. Suitably the heterocycloalkyl is a heterocyclopropyl, heterocyclobutyl, heterocyclopentyl or heterocyclohexyl ring such as a heterocyclohexyl ring.

Suitably, the heterocyclopentyl ring is tetrahydrofuranyl or pyrrolidinyl. Suitably, the heterocyclohexyl ring is tetrahydropyranyl or piperidinyl. Any nitrogen atom(s) in the C₃₋₆heterocycloalkyl ring may be substituted (such as one nitrogen atom is substituted), for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃alkylOC₁₋₂alkyl such as C(O)CH₂OCH₃, and C₁₋₂alkylC(O)OC₁₋₄alkyl such as CH₂C(O)OCH₂CH₃. Suitably, any nitrogen atom in the C₁₋₃heterocycloalkyl ring is not substituted.

In a sixth embodiment, the substituent is C₁₋₃alkyleneOC₁₋₃alkyl, in particular C₁₋₂alkyleneOC₁₋₂alkyl such as C₁alkyleneOC₁alkyl, C₂alkyleneOC₁alkyl, C₁alkyleneOC₂alkyl or C₂alkyleneOC₂alkyl.

In a seventh embodiment, the substituent is halo, in particular fluoro or chloro such as chloro.

In an eighth embodiment, the substituent is OC₁₋₃haloalkyl. Suitably the OC₁₋₃alkyl group is substituted by one two or three, such as one, halogen atom. Suitably, the halogen atom is fluoro or chloro such as fluoro. Suitably, the substituent is OC₁haloalkyl such as OCF₃. Suitably, the substituent is OC₂haloalkyl such as OCH₂CF₃.

In a ninth embodiment, the substituent is OC₀₋₂alkyleneC₃₋₆cycloalkyl, such as OC₃₋₆cycloalkyl, OC₁alkyleneC₃₋₆cycloalkyl or OC₂alkyleneC₃₋₆cycloalkyl.

In a tenth embodiment, the substituent is OC₀₋₂alkyleneC₃₋₆heterocycloalkyl such as OC₀₋₂alkyleneC₃heterocycloalkyl, C₀₋₂alkyleneC₄heterocycloalkyl, OC₀₋₂alkyleneC₅heterocycloalkyl, OC₀₋₂alkyleneC₆heterocycloalkyl, OC₀alkyleneC₃₋₆heterocycloalkyl, OC₁alkyleneC₃₋₆heterocycloalkyl and OC₂alkyleneC₃₋₆heterocycloalkyl. Suitably the heterocycloalkyl is a heterocyclopropyl, heterocyclobutyl, heterocyclopentyl or heterocyclohexyl ring such as a heterocyclohexyl ring. Suitably, the heterocyclopentyl ring is tetrahydrofuranyl or pyrrolidinyl. Suitably, the heterocyclohexyl ring is tetrahydropyranyl or piperidinyl. Any nitrogen atom(s) (such as one nitrogen atom) in the C₃₋₆heterocycloalkyl ring may be substituted, for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄ alkyl, C(O)NHC₁₋₄alkylaryl such as C(O)NHBz, an Fmoc group, C(O)C₁₋₄haloalkyl, C(O)OC₁₋₄ haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃ alkylOC₁₋₂alkyl such as C(O)CH₂OCH₃, and C₁₋₂alkylC(O)OC₁₋₄alkyl such as CH₂C(O)OCH₂CH₃. Suitably, any nitrogen atom in the C₃₋₆heterocycloalkyl ring is not substituted.

In an eleventh embodiment, the substituent is OC₁₋₃alkyl, such as OCH₃ or OCH₂CH₃.

In a twelfth embodiment, the substituent is NR₂₁ₐR₂₂ₐ wherein R₂₁ₐ and R₂₂ₐ are defined elsewhere herein.

In an embodiment the substituent is oxo.

In another embodiment the substituent is OH.

Suitably, the one or two substituents, in particular one substituent, are independently selected from the group consisting of C₁₋₃alkyl, oxo, OH, C₁₋₃alkylOH, C₁₋₃haloalkyl, halo, OC₁₋₃haloalkyl, OC₁₋₃alkyl and NR₂₁ₐR₂₂ₐ.

More suitably, the substituent is independently selected from the group consisting of oxo, OH, halo, OC₁₋₃alkyl and NR₂₁ₐR₂₂ₐ.

Most suitably, the substituent is independently selected from the group consisting of oxo, OH, fluoro and NR₂₁ₐR₂₂ₐ.

When the substituent is NR₂₁ₐR₂₂ₐ, in one embodiment R₂₁ₐ is H. In a second embodiment R₂₁ₐ is C₁₋₅alkyl, such as methyl, ethyl or propyl, especially methyl. In a third embodiment R₂₁ₐ is C(O)C₁₋₅alkyl, such as C(O)CH₃. In a fourth embodiment R₂₁ₐ is C(O)OC₁₋₂alkyl, such as C(O)OCH₃ or C(O)Otert-butyl. In a fifth embodiment R₂₁ₐ is C₁₋₃alkylOC₁₋₂alkyl such as C₁alkylOC₁alkyl, C₂alkylOC₁alkyl or C₃alkylOC₁alkyl e.g. C₂alkylOC₁alkyl. In a sixth embodiment, R₂₁ₐ is C₁₋₄haloalkyl, such as CF₃, CH₂CF₃ or CH₂CHF₂ e.g. CH₂CHF₂. In a seventh embodiment R₂₁ₐ is C₄₋₆heterocycloalkyl, such as oxetanyl, tetrahydrofuranyl or tetrahydropyranyl e.g. oxetanyl, in particular 3-oxetanyl.

When the substituent is NR₂₁ₐR₂₂ₐ, in one embodiment R₂₂ₐ is H. In a second embodiment R₂₂ₐ is methyl.

Suitably, R₂₁ₐ is C(O)OCH₃ and R₂₂ₐ is H. Suitably, R₂₁ₐ is C(O)CH₃ and R₂₂ₐ is H. Suitably, R₂₁ₐ and R₂₂ₐ are both CH₃. Suitably, R₂₁ₐ and R₂₂ₐ are both H.

Alternatively, R₄ₐₐ and R₅ₐₐ suitably together with the carbon atom to which they are attached form a C₃₋₆cycloalkyl and one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl.

In one embodiment the C₃₋₆cycloalkyl formed by R₄ₐₐ and R₅ₐₐ together with the carbon atom to which they are attached is unsubstituted. In a second embodiment the C₃₋₆cycloalkyl formed by R₄ₐₐ and R₅ₐₐ together with the carbon atom to which they are attached is substituted by one or two substituents, in particular one substituent. Suitably, each substituent being independently selected from the group consisting of C₁₋₂alkyl or OCH₃.

Suitably one of the carbons of the C₃₋₆cycloalkyl formed by R₄ₐₐ and R₅ₐₐ together with the carbon atom to which they are attached is a C₄₋₆cycloalkyl. Suitably the further C₃₋₆heterocycloalkyl is an oxygen containing C₃₋₆heterocycloalkyl.

Alternatively, R₄ₐₐ and R₅ₐₐ suitably together with the carbon atom to which they are attached form a C₃₋₆heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐₐ and R₅ₐₐ together with the carbon atom to which they are attached may be substituted by one or two substituents, each substituent being independently selected from the group consisting of C₁₋₃ alkyl or OC₁₋₃alkyl.

In one embodiment the C₃₋₆heterocycloalkyl formed by R₄ₐₐ and R₅ₐₐ together with the carbon atom to which they are attached is unsubstituted. In a second embodiment the C₃₋₆heterocycloalkyl formed by R₄ₐₐ and R₅ₐₐ together with the carbon atom to which they are attached is substituted by one or two substituents, in particular one substituent. Suitably, each substituent being independently selected from the group consisting of C₁₋₂alkyl or OCH₃.

In an embodiment, R₄ₐₐ and R₅ₐₐ together with the carbon atom to which they are attached form a C₃₋₆heterocycloalkyl comprising one nitrogen atom, wherein said nitrogen atom is substituted by —S(O)₂R₂₉ₐ.

Suitably, the C₃₋₆heterocycloalkyl is selected from the group consisting of azirdinyl, azetidinyl, pyrrolidinyl and piperidinyl such as piperidinyl.

Suitably, when the C₃₋₆heterocycloalkyl is piperidinyl, the nitrogen atom is in the 4-position relative to the quaternary carbon:

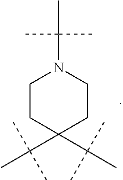

The C₃₋₆heterocycloalkyl may be other groups as defined elsewhere herein.

In an embodiment, R₂₉ₐ is C₁₋₃alkyl, C₀₋₂alkyleneC₃₋₅cycloalkyl which cycloalkyl is optionally substituted by CH₃, or CF₃. In one embodiment, R₂₉ₐ is C₁₋₃alkyl such as methyl. In another embodiment, R₂₉ₐ is C₀₋₂alkyleneC₃₋₅cycloalkyl which cycloalkyl is optionally substituted by CH₃. In some embodiments, R₂₉ₐ is C₀₋₂alkyleneC₃₋₅cycloalkyl. In other embodiments, R₂₉ₐ is C₀₋₂alkyleneC₃₋₅cycloalkyl which cycloalkyl is substituted by CH₃. R₂₉ₐ may be C₃₋₅cycloalkyl, which cycloalkyl is optionally substituted by CH₃. R₂₉ₐ may be C₁alkyleneC₃₋₅cycloalkyl, which cycloalkyl is optionally substituted by CH₃. R₂₉ₐ may be C₂alkyleneC₃₋₅cycloalkyl, which cycloalkyl is optionally substituted by CH₃. R₂₉ₐ may be C₀₋₂alkyleneC₃cycloalkyl, which cycloalkyl is optionally substituted by CH₃. R₂₉ₐ may be C₀₋₂alkyleneC₄cycloalkyl, which cycloalkyl is optionally substituted by CH₃. R₂₉ₐ may be C₀₋₂alkyleneC₅cycloalkyl, which cycloalkyl is optionally substituted by CH₃. Suitably, where C₀₋₂alkyleneC₃₋₅cycloalkyl is optionally substituted by CH₃, the CH₃ is at the point of attachment of the C₃₋₅cycloalkyl to the C₀₋₂alkylene. In another embodiment, R₂₉ₐ is CF₃. In another embodiment, R₂₉ₐ is N(C₁₋₃alkyl)₂ such as N(CH₃)₂. In another embodiment, R₂₉ₐ is a 5 or 6 membered heteroaryl wherein the 5 or 6 membered heteroaryl is optionally substituted by methyl. In one embodiment, the 5 or 6 membered heteroaryl is substituted by methyl. In one embodiment, the 5 or 6 membered heteroaryl is not substituted by methyl. In one embodiment, R₂₉ₐ is a 5-membered heteroaryl such as pyrrolyl, furanyl, thiophenyl, imidazolyl, pyrazolyl, oxazolyl, isoxazolyl, thiazolyl, isothiazolyl, such as pyrazolyl. Suitably the pyrazolyl is substituted by methyl. In another embodiment, R₂₉ₐ is a 6-membered heteroaryl such as pyridinyl, pyridazinyl, pyrimidinyl or pyrazinyl.

In another embodiment, R₄ₐ and R₅ₐ are R_{4ba} and R_{5ba}.

In one embodiment, R_{4ba} and R_{5ba} together with the carbon atom to which they are attached form a C₃₋₆cycloalkyl, such as cyclopropyl, cyclobutyl or cyclopentyl in particular cyclopropyl or cyclopentyl. In a second embodiment, R_{4ba} and R_{5ba} together with the carbon atom to which they are attached form a C₃₋₆heterocycloalkyl, such as a heterocyclohexyl, in particular a tetrahydropyranyl. Any nitrogen atom such as one nitrogen atom in the C₃₋₆heterocycloalkyl ring may be substituted, for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃alkylOC₁₋₂alkyl such as C(O)CH₂OCH₃, and C₁₋₂alkylC(O)OC₁₋₄ alkyl such as CH₂C(O)OCH₂CH₃. Suitably, any nitrogen atom in the C₃₋₆heterocycloalkyl ring is not substituted. In a third embodiment, R_{4ba} is C₁₋₆alkyl, in particular C₁₋₄alkyl such as methyl, ethyl, propyl (n-propyl or isopropyl) or butyl (n-butyl, isobutyl, sec-butyl or tert-buty). In a fourth embodiment, R_{4ba} is C₁₋₃alkyleneOC₁₋₃alkyl, in particular C₁₋₂alkyleneOC₁₋₂alkyl such as C₁alkyleneOC₁alkyl, C₂alkyleneOC₁alkyl, C₁alkyleneOC₂alkyl or C₂alkyleneOC₂alkyl. In a fifth embodiment, R_{4ba} is H. In a sixth embodiment, R_{4ba} is halo, such as chloro or fluoro, especially fluoro. In a seventh embodiment, R_{4ba} is C₁₋₆haloalkyl, such as CF₃ or CH₂CF₃. In an eighth embodiment, R_{4ba} is C₀₋₂alkyleneC₃₋₆cycloalkyl such as C₃₋₆cycloalkyl, C₁alkyleneC₃₋₆cycloalkyl, C₂alkyleneC₃₋₆cycloalkyl, C₀₋₂alkyleneC₃cycloalkyl, C₀₋₂alkyleneC₄cycloalkyl, C₀₋₂alkyleneC₅cycloalkyl or C₀₋₂alkyleneC₆cycloalkyl. In a ninth embodiment, R_{4ba} is C₀₋₂alkyleneC₃₋₆heterocycloalkyl such as C₃₋₆heterocycloalkyl, C₁alkyleneC₃₋₆heterocycloalkyl, C₂alkyleneC₃₋₆heterocycloalkyl, C₀₋₂alkyleneC₃heterocycloalkyl, C₀₋₂alkyleneC₄hetero-cycloalkyl, C₀₋₂alkyleneC₅heterocycloalkyl or C₀₋₂alkyleneC₆heterocycloalkyl. Suitably the heterocycloalkyl is a heterocyclopropyl, heterocyclobutyl, heterocyclopentyl or heterocyclohexyl ring such as a heterocyclohexyl ring. Suitably, the heterocyclopentyl ring is tetrahydrofuranyl or pyrrolidinyl.

Suitably, the heterocyclohexyl ring is tetrahydropyranyl or piperidinyl. Any nitrogen atom such as one nitrogen atom in the C₃₋₆heterocycloalkyl ring may be substituted, for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃alkylOC₁₋₂alkyl such as C(O)CH₂OCH₃, and C₁₋₂alkylC(O)OC₁₋₄alkyl such as CH₂C(O)OCH₂CH₃. Suitably, any nitrogen atom in the C₃₋₆heterocycloalkyl ring is not substituted. In a tenth embodiment, R_{4ba} is C₁₋₆alkylOH, such as CH₂OH or CH₂CH₂OH. In an eleventh embodiment, R_{4ba} is OC₁₋₆haloalkyl, such as OC₁₋₄haloalkyl, such as OCF₃ or OCHF₂. In a twelfth embodiment, R_{4ba} is OC₀₋₂alkyleneC₃₋₆cycloalkyl such as OC₃₋₆cycloalkyl, OC₁alkyleneC₃₋₆cycloalkyl, OC₂alkyleneC₃₋₆cycloalkyl, OC₀₋₂alkyleneC₃cycloalkyl, OC₀₋₂alkyleneC₄cycloalkyl, OC₀₋₂alkyleneC₅cycloalkyl or OC₀₋₂alkyleneC₆cycloalkyl. In a thirteenth embodiment, R_{4ba} is OC₁₋₆alkyl, in particular OC₁₋₄alkyl such as methoxy, ethoxy, propoxy (n-propoxy or isopropoxy) or butoxy (n-butoxy, isobutoxy, sec-butoxy or tert-butoxy). In a fourteenth embodiment, R_{4ba} is OC₀₋₂alkyleneC₃₋₆heterocycloalkyl such as OC₃₋₆heterocycloalkyl, OC₁alkyleneC₃₋₆heterocycloalkyl, OC₂alkyleneC₃₋₆heterocycloalkyl, OC₀₋₂alkyleneC₃heterocycloalkyl, OC₀₋₂alkyleneC₄hetero-cycloalkyl, OC₀₋₂alkyleneC₅heterocycloalkyl or OC₀₋₂alkyleneC₆heterocycloalkyl. Suitably the heterocycloalkyl is a heterocyclopropyl, heterocyclobutyl, heterocyclopentyl or heterocyclohexyl ring such as a heterocyclohexyl ring. Suitably, the heterocyclopentyl ring is tetrahydrofuranyl or pyrrolidinyl. Suitably, the heterocyclohexyl ring is tetrahydropyranyl or piperidinyl. Any nitrogen atom such as one nitrogen atom in the C₃₋₆heterocycloalkyl ring may be substituted, for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃alkylOC₁₋₂alkyl such as C(O)CH₂OCH₃, and C₁₋₂alkylC(O)OC₁₋₄alkyl such as CH₂C(O)OCH₂CH₃. Suitably, any nitrogen atom in the C₃₋₆heterocycloalkyl ring is not substituted. In a fifteenth embodiment, R_{4ba} is NR₂₁ₐR₂₂ₐ.

When Aₐ is —NHC(═O)— or —C(═O)NH—, suitably, R_{4ba} is H, C₁₋₆alkyl, C₁₋₆haloalkyl, C₁₋₆alkylOH, C₀₋₂alkyleneC₃₋₆cycloalkyl, C₀₋₂alkyleneC₃₋₆heterocycloalkyl, C₁₋₃alkyleneOC₁₋₃alkyl, or R_{4ba} and R_{5ba} together with the carbon atom to which they are attached form a C₃₋₆cycloalkyl or C₃₋₆heterocycloalkyl. When Aₐ is —NHC(═O)—, suitably R_{4ba} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl or NR₂₁ₐR₂₂ₐ.

When Aₐ is —NH—, —CH₂NH— or —NHCH₂—, suitably, R_{4ba} is H, C₁₋₆alkyl, C₁₋₆haloalkyl, C₁₋₆alkylOH, C₀₋₂alkyleneC₃₋₆cycloalkyl, C₀₋₂alkyleneC₃₋₆heterocycloalkyl, C₁₋₃alkyleneOC₁₋₃alkyl, or R_{4ba} and R_{5ba} together with the carbon atom to which they are attached form a C₃₋₆cycloalkyl or C₃₋₆heterocycloalkyl. When Aₐ is —NHCH₂—, suitably R_{4ba} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl or NR₂₁ₐR₂₂ₐ.

Suitably R_{4ba} is H, fluoro, CH₃, ethyl, OCH₃ or CH₂CH₂OCH₃, such as fluoro, ethyl, OCH₃ or CH₂CH₂OCH₃.

Suitably R_{4ba} is H, CH₃, ethyl or CH₂CH₂OCH₃, in particular CH₃ or ethyl.

Suitably R_{4ba} and R_{5ba} together with the carbon atom to which they are attached form a cyclopropyl or cyclopentyl, in particular a cyclopentyl.

Suitably R_{4ba} and R_{5ba} together with the carbon atom to which they are attached form a heterocyclohexyl, such as tetrahydropyranyl or piperidinyl, especially tetrahydropyranyl. Any nitrogen atom such as one nitrogen atom in the C₃₋₆heterocycloalkyl ring may be substituted, for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃alkylOC₁₋₂alkyl such as C(O)CH₂OCH₃, and C₁₋₂alkylC(O)OC₁₋₂alkyl such as CH₂C(O)OCH₂CH₃. Suitably, any nitrogen atom in the C₃₋₆heterocycloalkyl ring is not substituted.

Suitably R_{4ba} and R_{5ba} together with the carbon atom to which they are attached form a heterocyclobutyl, such as azetidinyl. Any nitrogen atom such as one nitrogen atom in the C₃₋₆heterocycloalkyl ring may be substituted, for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄ haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃alkylOC₁₋₂alkyl such as C(O)CH₂OCH₃, and C₁₋₂alkylC(O)OC₁₋₄alkyl such as CH₂C(O)OCH₂CH₃. Suitably, any nitrogen atom in the C₃₋₆heterocycloalkyl ring is not substituted.

When R_{4ba} is NR₂₁ₐR₂₂ₐ, in one embodiment R₂₁ₐ is H. In a second embodiment R₂₁ₐ is C₁₋₅alkyl, such as methyl, ethyl or propyl, especially methyl. In a third embodiment R₂₁ₐ is C(O)C₁₋₅alkyl, such as C(O)CH₃. In a fourth embodiment R₂₁ₐ is C(O)OC₁₋₅alkyl, such as C(O)OCH₃ or C(O)Otert-butyl. In a fifth embodiment R₂₁ₐ is C₁₋₃alkylOC₁₋₂alkyl such as C₁alkylOC₁alkyl, C₂alkylOC₁alkyl or C₃alkylOC₁alkyl e.g. C₂alkylOC₁alkyl. In a sixth embodiment, R₂₁ₐ is C₁₋₄haloalkyl, such as CF₃, CH₂CF₃ or CH₂CHF₂ e.g. CH₂CHF₂. In a seventh embodiment R₂₁ₐ is C₄₋₆heterocycloalkyl, such as oxetanyl, tetrahydrofuranyl or tetrahydropyranyl e.g. oxetanyl, in particular 3-oxetanyl.

When R_{4ba} is NR₂₁ₐR₂₂ₐ, in one embodiment R₂₂ₐ is H. In a second embodiment R₂₂ₐ is methyl.

For example, R_{4ba} is NH₂, N(CH₃)₂, NHC(O)CH₃, NHC(O)OCH₃, NHC(O)Otert-butyl and CH₂CH₂OH, especially, N(CH₃)₂, NHC(O)CH₃, NHC(O)OCH₃.

Suitably, R₂₁ₐ is C(O)OCH₃ and R₂₂ₐ is H. Suitably, R₂₁ₐ is C(O)CH₃ and R₂₂ₐ is H. Suitably, R₂₁ₐ and R₂₂ₐ are both CH₃. Suitably, R₂₁ₐ and R₂₂ₐ are both H.

In one embodiment R_{5ba} is C₁₋₆alkyl, in particular C₁₋₄alkyl such as methyl, ethyl, propyl (n-propyl or isopropyl) or butyl (n-butyl, isobutyl, sec-butyl or tert-butyl). In a second embodiment R_{5ba} is C₁₋₃alkyleneOC₁₋₃alkyl, in particular C₁₋₂alkyleneOC₁₋₂alkyl such as C₁alkyleneOC₁alkyl, C₂alkyleneOC₁alkyl, C₁alkyleneOC₂alkyl or C₂alkyleneOC₂alkyl. In a third embodiment R_{5ba} is H. In a fourth embodiment, R_{5ba} is halo, such as chloro or fluoro, especially fluoro. In a fifth embodiment, R_{5ba} is C₁₋₆haloalkyl, such as CF₃ or CH₂CF₃. In a sixth embodiment, R_{5ba} is C₀₋₂alkyleneC₃₋₆cycloalkyl such as C₃₋₆cycloalkyl, C₁alkyleneC₃₋₆cycloalkyl, C₂alkyleneC₃₋₆cycloalkyl, C₀₋₂alkyleneC₃cycloalkyl, C₀₋₂alkyleneC₄cycloalkyl, C₀₋₂alkyeneC₅cycloalkyl or C₀₋₂alkyleneC₆cycloalkyl. In a seventh embodiment, R_{5ba} is C₀₋₂alkyleneC₃₋₄heterocycloalkyl such as C₃₋₆heterocycloalkyl, C₁alkyleneC₃₋₆heterocycloalkyl, C₂alkyleneC₃₋₆heterocycloalkyl, C₀₋₂alkyleneC₆heterocycloalkyl, C₀₋₂alkyleneC₄hetero-cycloalkyl, C₀₋₂alkyleneC₅heterocycloalkyl or C₀₋₂alkyleneC₆heterocycloalkyl. Suitably the heterocycloalkyl is a heterocyclopropyl, heterocyclobutyl, heterocyclopentyl or heterocyclohexyl ring such as a heterocyclohexyl ring. Suitably, the heterocyclopentyl ring is tetrahydrofuranyl or pyrrolidinyl. Suitably, the heterocyclohexyl ring is tetrahydropyranyl or piperidinyl. Any nitrogen atom such as one nitrogen atom in the C₃₋₆heterocycloalkyl ring may be substituted, for example by C₁₋₄alkyl, C(O)H, C(O)C₁₋₄ alkyl, C(O)OC₁₋₄alkyl, C(O)OC₁₋₄alkylaryl such as C(O)OBz, C(O)NHC₁₋₄alkyl, C(O)NHC₁₋₄ alkylaryl such as C(O)NHBz, an Fmoc group, C(O)C₁₋₄haloalkyl, C(O)OC₁₋₄haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃alkylOC₁₋₂alkyl such as C(O)CH₂OCH₃, and C₁₋₂alkylC(O)OC₁₋₄alkyl such as CH₂C(O)OCH₂CH₃. Suitably, any nitrogen atom in the C₃₋₆heterocycloalkyl ring is not substituted. In an eighth embodiment, R_{5ba} is C₁₋₆alkylOH, such as CH₂OH or CH₂CH₂OH. In a ninth embodiment, R_{5ba} is OC₁₋₆haloalkyl, such as OC₁₋₄haloalkyl, such as OCF₃ or OCHF₂. In a tenth embodiment, R_{5ba} is OC₀₋₂alkyleneC₃₋₆cycloalkyl such as OC₃₋₆cycloalkyl, OC₁alkyleneC₃₋₆cycloalkyl, OC₂alkyleneC₃₋₆cycloalkyl, OC₀₋₂alkyleneC₃cycloalkyl, OC₀₋₂alkyleneC₄cycloalkyl, OC₀₋₂alkyleneC₅cycloalkyl or OC₀₋₂alkyleneC₆cycloalkyl. In an eleventh embodiment, R_{5ba} is OC₁₋₆alkyl, in particular OC₁₋₄alkyl such as methoxy, ethoxy, propoxy (n-propoxy or isopropoxy) or butoxy (n-butoxy, isobutoxy, sec-butoxy or tert-butoxy). In a twelfth embodiment, R_{5ba} is OC₀₋₂alkyleneC₃₋₆heterocycloalkyl such as OC₃₋₆heterocycloalkyl, OC₁alkyleneC₃₋₆heterocycloalkyl, OC₂alkyleneC₃₋₆heterocycloalkyl, OC₀₋₂alkyleneC₃heterocycloalkyl, C₀₋₂alkyleneC₄hetero-cycloalkyl, OC₀₋₂alkyleneC₅heterocycloalkyl or OC₀₋₂alkyleneC₆heterocycloalkyl. Suitably the heterocycloalkyl is a heterocyclopropyl, heterocyclobutyl, heterocyclopentyl or heterocyclohexyl ring such as a heterocyclohexyl ring. Suitably, the heterocyclopentyl ring is tetrahydrofuranyl or pyrrolidinyl. Suitably, the heterocyclohexyl ring is tetrahydropyranyl or piperidinyl. Any nitrogen atom such as one nitrogen atom in the C₃₋₆heterocycloalkyl ring may be substituted, for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₄haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃alkylOC₁₋₂alkyl such as C(O)CH₂OCH₃, and C₁₋₂alkylC(O)OC₁₋₄alkyl such as CH₂C(O)OCH₂CH₃. Suitably, any nitrogen atom in the C₃₋₆heterocycloalkyl ring is not substituted. In a thirteenth embodiment, R_{5ba} is NR₂₁ₐR₂₂ₐ.

When Aₐ is —NHC(═O)— or —C(═O)NH—, suitably, R_{5ba} is H, C₁₋₆alkyl, C₁₋₆haloalkyl, C₁₋₆alkylOH, C₀₋₂alkyleneC₃₋₆cycloalkyl, C₀₋₂alkyleneC₃₋₆heterocycloalkyl, C₁₋₃alkyleneOC₁₋₃alkyl, or R_{4ba} and R_{5ba} together with the carbon atom to which they are attached form a C₃₋₆cycloalkyl or C₃₋₆heterocycloalkyl. When Aₐ is —NHC(═O)—, suitably R_{5ba} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl or NR₂₁ₐR₂₂ₐ.

When Aₐ is —NH—, —CH₂NH— or —NHCH₂—, suitably, R_{5ba} is H, C₁₋₆alkyl, C₁₋₆haloalkyl, C₁₋₆alkylOH, C₀₋₂alkyleneC₃₋₆cycloalkyl, C₀₋₂alkyleneC₃₋₆heterocycloalkyl, C₁₋₃alkyleneOC₁₋₃alkyl, or R_{4ba} and R_{5ba} together with the carbon atom to which they are attached form a C₃₋₆cycloalkyl or C₃₋₆heterocycloalkyl. When Aₐ is —NHCH₂—, suitably R_{5ba} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl or NR₂₁ₐR₂₂ₐ.

When R_{5ba} is NR₂₁ₐR₂₂ₐ, in one embodiment R₂₁ₐ is H. In a second embodiment R₂₁ₐ is C₁₋₅alkyl, such as methyl, ethyl or propyl, especially methyl. In a third embodiment R₂₁ₐ is C(O)C₁₋₅alkyl, such as C(O)CH₃. In a fourth embodiment R₂₁ₐ is C(C)OC₁₋₅alkyl, such as C(O)OCH₃ or C(O)Otert-butyl. In a fifth embodiment R₂₁ₐ is C₁₋₃alkylOC₁₋₂alkyl such as C₁alkylOC₁alkyl, C₂alkylOC₁alkyl or C₃alkylOC₁alkyl e.g. C₂alkylOC₁alkyl. In a sixth embodiment, R₂₁ₐ is C₁₋₄ haloalkyl, such as CF₃, CH₂CF₃ or CH₂CHF₂ e.g. CH₂CHF₂. In a seventh embodiment R₂₁ₐ is C₄₋₆heterocycloalkyl, such as oxetanyl, tetrahydrofuranyl or tetrahydropyranyl e.g. oxetanyl, in particular 3-oxetanyl.

When R_{5ba} is NR₂₁ₐR₂₂ₐ, in one embodiment R₂₂ₐ is H. In a second embodiment R₂₂ₐ is methyl.

For example, R_{5ba} is NH₂, N(CH₃)₂, NHC(O)CH₃, NHC(O)OCH₃, NHC(O)Otert-butyl and CH₂CH₂OH, especially, N(CH₃)₂, NHC(O)CH₃, NHC(O)OCH₃.

Suitably, R₂₁ₐ is C(O)OCH₃ and R₂₂ₐ is H. Suitably, R₂₁ₐ is C(O)CH₃ and R₂₂ₐ is H. Suitably, R₂₁ₐ and R₂₂ₐ are both CH₃. Suitably, R₂₁ₐ and R₂₂ₐ are both H.

Suitably R_{5ba} is H, F, CH₃ or ethyl such as H, CH₃ or ethyl.

Suitably R_{4ba} is H, CH₃, ethyl or CH₂CH₂OCH₃ and R_{5ba} is H, CH₃ or ethyl, in particular R_{4ba} is CH₃, or ethyl and R_{5ba} is H, methyl or ethyl. For example, R_{4ba} and R_{5ba} are H, R_{4ba} and R_{5ba} are methyl, R_{4ba} and R_{5ba} are ethyl or R_{4ba} is CH₂CH₂OCH₃ and R_{5ba} is H.

Suitably, R_{4ba} is F and R_{5ba} is ethyl.

Suitably, R_{4ba} is F and R_{5ba} is F.

Suitably, R_{4ba} is ethyl and R_{5ba} is H.

Suitably R_{4ba} and R_{5ba} are arranged in the following configuration:

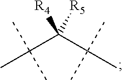

wherein R₄ and R₅ include variables R₄ₐ, R₅ₐ, R₄ₐₐ, R₅ₐₐ, R_{4ba} and R_{5ba}.

In one embodiment Ar1a is a 6-membered aryl, i.e. phenyl. In a second embodiment Ar1a is a 6-membered heteroaryl, in particular containing one nitrogen atom (pyridyl) or two nitrogen atoms (pyridazinyl, pyrimidinyl or pyrazinyl).

In particular Ar1a is phenyl, 2-pyridyl or 3-pyridyl, such as phenyl or 2-pyridyl.

In one embodiment R₁₀ₐ is H. In a second embodiment R₁₀ₐ is halo, for example fluoro or chloro. In a third embodiment R₁₀ₐ is C₁₋₃alkyl such as C₁₋₂alkyl, such as CH₃ or ethyl. In a fourth embodiment R₁₀ₐ is OC₁₋₂alkyl, such as OCH₃ or ethoxy. In a fifth embodiment R₁₀ₐ is OC₁₋₂haloalkyl, such as OCF₃. In a sixth embodiment R₁₀ₐ is CN. In a seventh embodiment, R₁₀ₐ is C₁₋₂ haloalkyl such as CF₃.

Suitably R₁₀ₐ is H, fluoro, chloro, CH₃, CF₃, OCH₃, OCF₃ or CN, such as H, fluoro, chloro, CH₃, OCH₃, OCF₃ or CN, in particular H, fluoro, chloro, OCH₃, OCF₃ or CN especially H or fluoro.

Suitably, R₁₀ₐ is H, F or CH₃.

In one embodiment R₁₁ₐ is H. In a second embodiment R₁₁ₐ is F. In a third embodiment, R₁₁ₐ is C₁₋₂alkyl such as CH₃ or Et, such as CH₃. In a fourth embodiment R₁₁ₐ is OCH₃. In a fifth embodiment, R₁₁ₐ is Cl. In a sixth embodiment, R₁₁ₐ is Et. In a seventh embodiment, R₁₁ₐ is CF₃. In an eighth embodiment, R₁₁ₐ is CN.

Suitably, R₁₁ₐ is H, F, CH₃ or OCH₃, such as H, F or CH₃, such as H or F, such as H.

In one embodiment, R₁₀ₐ is in the ortho position with respect to group Aa. In another embodiment, R₁₀ₐ is in the meta position with respect to group Aa. Suitably R₁₀ₐ is in the ortho position with respect to group Aa.

In one embodiment, R₁₁ₐ is in the ortho position with respect to group Aa. In another embodiment, R₁₁ₐ is in the meta position with respect to group Aa. Suitably R₁₁ₐ is in the ortho position with respect to group Aa.

In one embodiment Ar2a is a 6-membered aryl, i.e. phenyl. In a second embodiment Ar2a is a 6-membered heteroaryl, in particular containing one nitrogen atom (pyridyl) or two nitrogen atoms (pyridazinyl, pyrimidinyl or pyrazinyl).

In particular Ar2a is 3-pyridyl or 2,5-pyrazinyl, especially 2,5-pyrazinyl.

In one embodiment R₁₂ₐ is H. In a second embodiment R₁₂ₐ is halo, for example fluoro or chloro. In a third embodiment R₁₂ₐ is C₁₋₄alkyl, such as methyl, ethyl, propyl (n-propyl or isopropyl) or butyl (n-butyl, isobutyl, sec-butyl or tert-butyl). In a fourth embodiment R₁₂ₐ is OC₁₋₄alkyl, such as OCH₃, ethoxy, isopropoxy or n-propoxy. In a fifth embodiment R₁₂ₐ is OC₀₋₂alkyleneC₃₋₅cycloalkyl, such as OC₃₋₅cycloalkyl (e.g. cyclopropoxy or cyclobutoxy), OC₁alkyleneC₃₋₅cycloalkyl or OC₂alkyleneC₃₋₅cycloalkyl. In a sixth embodiment R₁₂ₐ is CN. In a seventh embodiment R₁₂ₐ is C₁₋₄haloalkyl, such as CF₃. In an eighth embodiment R₁₂ₐ is OC₁₋₄haloalkyl, such as OCF₃, OCHF₂ or OCH₂CF₃. In a ninth embodiment, R₁₂ₐ is C₂₋₄alkenyl such as C(═CH₂)CH₃. In a tenth embodiment, R₁₂ₐ is C₀₋₂alkyleneC₃₋₅cycloalkyl such as C₃₋₅cycloalkyl, C₁alkyleneC₃₋₅cycloalkyl, C₂alkyleneC₃₋₅cycloalkyl, C₀₋₂alkyleneC₃cycloalkyl, C₀₋₂alkyleneC₄cycloalkyl or C₀₋₂alkyleneC₅cycloalkyl. In an eleventh embodiment, R₁₂ₐ is hydroxy. In a twelfth embodiment, R₁₂ₐ is C₁₋₄alkylOH such as CH₂OH. In a thirteenth embodiment, R₁₂ₐ is SO₂C₁₋₂alkyl such as SO₂CH₃. In a fourteenth embodiment, R₁₂ₐ is C(O)N(C₁₋₂alkyl)₂ such as C(O)N(CH₃)₂. In a fifteenth embodiment, R₁₂ₐ is NHC(O)C₁₋₃alkyl. In a sixteenth embodiment, R₁₂ₐ is NR₂₃ₐR₂₄ₐ. In a seventeenth embodiment, R₁₂ₐ is OCH₂CH₂N(CH₃)₂. In an eighteenth embodiment, R₁₂ₐ is a C₃₋₆heterocycloalkyl comprising one nitrogen located at the point of attachment to Ar2a. Suitably the heterocycloalkyl is a heterocyclopropyl, heterocyclobutyl, heterocyclopentyl or heterocyclohexyl ring such as a heterocyclohexyl ring. Suitably, the heterocyclopentyl ring is pyrrolidinyl. Suitably, the heterocyclohexyl ring is piperidinyl or piperazinyl. Any nitrogen atom such as one nitrogen atom in the C₃₋₆heterocycloalkyl ring may be substituted, for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃alkylOC₁₋₂alkyl such as C(O)CH₂OCH₃, and C₁₋₂alkylC(O)OC₁₋₄alkyl such as CH₂C(O)OCH₂CH₃. Suitably, any nitrogen atom in the C₃₋₆heterocycloalkyl ring is not substituted. In a nineteenth embodiment, R₁₂ₐ together with a nitrogen atom to which it is attached forms an N-oxide (N⁺—O⁻).

When Aa is —NHC(═O)— or —C(═O)NH—, suitably, R₁₂ₐ is attached to Ar2a in the ortho or meta position relative to Ar1a and R₁₂ₐ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃ₐR₂₄ₐ.

When Aa is —NHC(═O)—, suitably R₁₂ₐ may additionally be selected from CN, OCH₂CH₂N(CH₃)₂ and a C₃₋₆heterocycloalkyl comprising one nitrogen located at the point of attachment to Ar2a, or R₁₂ₐ together with a nitrogen atom to which it is attached forms an N-oxide (N⁺—O⁻).

When Aa is —NH—, —CH₂NH— or —NHCH₂—, suitably, R₁₂ₐ is attached to Ar2a in the ortho or meta position relative to Ar1a and R₁₂ₐ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃ₐR₂₄ₐ.

When Aa is —NH— or —NHCH₂—, suitably R₁₂ₐ may additionally be selected from CN, OCH₂CH₂N(CH₃)₂ and a C₃₋₆heterocycloalkyl comprising one nitrogen located at the point of attachment to Ar2a, or R₁₂ₐ together with a nitrogen atom to which it is attached forms an N-oxide (N⁺—O⁻).

R₁₂ₐ is suitably H, F, Cl, CH₃, OCH₃, OEt, OiPr, OCyclopropyl, CN, CF₃, OCHF₂ or OCH₂CF₃. In particular, R₁₂ₐ is Cl, CN, CF₃, OCHF₂, OCH₂CF₃, OCH₃, OEt, OiPr, OCyclopropyl, such as CF₃, OCHF₂, OCH₂CF₃, OCH₃, OEt, OiPr, OCyclopropyl, e.g. OEt.

R₁₂ₐ is suitably H, F, Cl, CH₃, iPr, OCH₃, OEt, OiPr, OCyclopropyl, CN, CF₃, OCHF₂, OCH₂CF₃, C₃cycloalkyl or C(═CH₂)CH₃. In particular, R₁₂ₐ is Cl, iPr, OCH₃, OEt, OiPr, OCyclopropyl, CN, CF₃, OCHF₂, OCH₂CF₃, C₃cycloalkyl or C(═CH₂)CH₃, such as Cl, OCH₃, OEt, OiPr, OCyclopropyl, CF₃, OCHF₂, OCH₂CF₃ or C₃cycloalkyl, e.g. OEt.

When Aa is —C(═O)NH—, suitably R₁₂ₐ is CF₃, OEt or OiPr, such as OEt or OiPr.

Suitably R₁₂ₐ is in the meta position of Ar2a. Alternatively, R₁₂ₐ is in the ortho position of Ar2a.

In one embodiment, R₁₃ₐ is H. In another embodiment, R₁₃ₐ is halo such as F or Cl, suitably F.

In one embodiment, R₁₃ₐ is in the ortho position with respect to Ar1a. In another embodiment, R₁₃ₐ is in the para position with respect to Ar1a. In another embodiment, R₁₃ₐ is in the meta position with respect to Ar1a.

In one embodiment, R₂₃ₐ is H. In another embodiment, R₂₃ₐ is C₁₋₂alkyl such as methyl.

In one embodiment, R₂₄ₐ is H. In another embodiment R₂₄ₐ is C₁₋₂alkyl such as methyl.

Suitably, R₂₃ₐ is H and R₂₄ₐ is ethyl. Suitably, R₂₃ₐ is CH₃ and R₂₄ₐ is CH₃.

Desirably, a compound of formula (I) does not include 2-(6-(methylsulfonamido)pyrazin-2-yl)-N-(4-(pyridin-3-yl)phenyl)acetamide.

In one embodiment, at least one of R₁₀ₐ, R₁₁ₐ, R₁₂ₐ and R₁₃ₐ is other than H.

Suitably, at least one of R₄ₐ, R₅ₐ, R₁₀ₐ, R₁₁ₐ, R₁₂ₐ and R₁₃ₐ is other than H.

More suitably, when R₁ₐ is methyl, at least one of R₄ₐ, R₅ₐ, R₁₀ₐ, R₁₁ₐ, R₁₂ₐ and R₁₃ₐ is other than H.

The present invention provides the compounds described in any one of Examples P226, P227, P228, P229, P230, P235, P242, P244, P248, P251, P254, P255, P256, P258, P260 and P261.

The present invention also provides the compounds described in any one of Examples P288, P289, P290, P291, P292, P293, P294, P295, P296, P297, P298, P299, P300, P301, P302, P303, P304, P305, P306, P307, P308, P309, P310, P311, P312, P313, P314, P315, P316, P317 and P318.

The present invention provides the following compounds:
1-(2-(cyclopropanesulfonamido)pyrimidin-4-yl)-N-(5-(6-ethoxypyrazin-2-yl)pyrdin-2-yl)-4-oxocyclohexanecarboxamide;
1-(2-(cyclopropanesulfonamido)pyrimidin-4-yl)-N-(5-(6-ethoxypyrazin-2-yl)pyrdin-2-yl)-4-hydroxycyclohexanecarboxamide;
1-(2-(cyclopropanesulfonamido)pyrimidin-4-yl)-N-(5-(6-ethoxypyrazin-2-yl)pyrdin-2-yl)-4-hydroxycyclohexanecarboxamide (diastereomer 1);
1-(2-(cyclopropanesulfonamido)pyrimidin-4-yl)-N-(5-(6-ethoxypyrazin-2-yl)pyrdin-2-yl)-4-hydroxycyclohexanecarboxamide (diastereomer 2);
1-(2-(cyclopropanesulfonamido)pyrimidin-4-yl)-4-(dimethylamino)-N-(5-(6-ethoxypyrazin-2-yl)pyrdin-2-yl)cyclohexane-1-carboxamide;
1-(2-(cyclopropanesulfonamido)pyrimidin-4-yl)-4-(dimethylamino)-N-(5-(6-ethoxypyrazin-2-yl)pyidin-2-yl)cyclohexane-1-carboxamide (diastereomer 1);
1-(2-(cyclopropanesulfonamido)pyrimidin-4-yl)-4-(dimethylamino)-N-(5-(6-ethoxypyrazin-2-yl)pyidin-2-yl)cyclohexane-1-carboxamide (diastereomer 2);
N-(4-(1-((4-(6-Ethoxypyrazin-2-yq)-2-fluorobenzyl)amino)propy)pyrimidin-2-yl)cyclopropanesulfonamide;
1-(2-(cyclopropanesulfonamido)pyrimidin-4-yl)-N-(5-(6-ethoxypyrazin-2-yl)pyrdin-2-yl)-4,4-difluorocyclohexane-1-carboxamide;
8-(2-(cyclopropanesulfonamido)pyrimidin-4-yl)-N-(5-(6-ethoxypyrazin-2-yl)pyrdin-2-yl)-1,4-dioxaspiro[4.5]decane-8-carboxamide;
4-(2-((N,N-dimethylsulfamoyl)amino)pyrimidin-4-yl)-N-(5-(6-ethoxypyrazin-2-yl)pyridin-2-yl)tetrahydro-2H-pyran-4-carboxamide;
4-(2-(cyclopropanesulfonamido)pyrimidin-4-yl)-N-(5-(6-ethoxypyrazin-2-yl)pyrdin-2-yl)-1-(methylsulfonyl)piperidine-4-carboxamide;
N-(4-(1-(((5-(6-ethoxypyrazin-2-yl)pyridin-2-yl)methyl)amino)cyclopropyl)pyrimidin-2-yl)cyclopropanesulfonamide;
N-(4-(1-((4-(6-ethoxypyrazin-2-yl)-2-fluorobenzyl)amino)cyclopropyl)pyrimidin-2-yl)cyclopropanesulfonamide;
N-(4-(4-(((4-(6-ethoxypyrazin-2-yl)phenyl)amino)methyl)tetrahydro-2H-pyran-4-yl)pyrimidin-2-yl)cyclopropanesulfonamide;
2-(2-(cyclopropanesulfonamido)pyrimidin-4-yl)-N-(5-(6-ethoxypyrazin-2-yl)pyrdin-2-yl)-5,8-dioxaspiro[3.4]octane-2-carboxamide;
1-(2-(cyclopropanesulfonamido)pyrimidin-4-yl)-N-(5-(6-ethoxypyrazin-2-yl)pyrdin-2-yl)-4-methoxycyclohexane-1-carboxamide; and
N-(4-(1-((4-(6-ethoxypyrazin-2-yl)phenyl)amino)propyl)pyrimidin-2-yl)cyclopropanesulfonamidearboxamide.

The present invention also provides the following compounds:
4-(2-(Cyclopropanesulfonamido)pyrimidin-4-yl)-N-(5-(6-ethoxypyrazin-2-yl)pyrdin-2-yl)-1-(2-methoxyacetyl)piperidine-4-carboxamide;
4-(2-(Cyclopropanesulfonamido)pyrimidin-4-yl)-N-(5-(6-ethoxypyrazin-2-yl)pyrdin-2-yl)-1-(ethylsulfonyl)piperidine-4-carboxamide;
4-(2-(Cyclopropanesulfonamido)pyrimidin-4-yl)-1-(cyclopropylsulfonyl)-N-(5-(6-ethoxypyrazin-2-yl)pyridin-2-yl)piperidine-4-carboxamide;
4-(2-(Cyclopropanesulfonamido)pyrimidin-4-yl)-1-(N,N-dimethylsulfamoyl)-N-(5-(6-ethoxypyrazin-2-yl)pyridin-2-yl)piperidine-4-carboxamide;
4-(2-(Cyclopropanesulfonamido)pyrimidin-4-yl)-N-(5-(6-ethoxypyrazin-2-yl)pyrdin-2-yl)-1-((trifluoromethyl)sulfonyl)piperidine-4-carboxamide;
4-(2-(Cyclopropanesulfonamido)pyrimidin-4-yl)-N-(5-(6-ethoxypyrazin-2-yl)pyrdin-2-yl)-1-((1-methyl-1H-pyrazol-3-yl)sulfonyl)piperidine-4-carboxamide;
1-(cyanomethyl)-4-(2-(cyclopropanesulfonamido)pyrimidin-4-yl)-N-(5-(6-ethoxypyrazin-2-yl)pyidin-2-yl)piperidine-4-carboxamide;
ethyl 2-(4-(2-(cyclopropanesulfonamido)pyrimidin-4-yl)-4-((5-(6-ethoxypyrazin-2-yl)pyridin-2-yl)carbamoyl)piperidin-1-yl)acetate;
N-(5-(6-ethoxypyrazin-2-yl)pyridin-2-yl)-4-(2-(ethylsulfonamido)pyrimidin-4-yl)-1-(2-methoxyacetyl)piperidine-4-carboxamide;
N-(5-(6-ethoxypyrazin-2-yl)pyridin-2-yl)-4(2-(ethylsulfonamido)pyrimidin-4-yl)-1-(methylsulfonyl)piperidine-4-carboxamide;
N-(5-(6-ethoxypyrazin-2-yl)pyridin-2-yl)-4-(2-(ethylsulfonamido)pyrimidin-4-yl)-1-(ethylsulfonyl)piperidine-4-carboxamide;
1-(Cyclopropylsulfonyl)-N-(5-(6-ethoxypyrazin-2-yl)pyridin-2-yl)-4-(2-(ethylsulfonamido)pyrimidin-4-yl)piperidine-4-carboxamide;
N-(5-(6-ethoxypyrazin-2-yl)pyridin-2-yl)-4-(2-(ethylsulfonamido)pyrimidin-4-yl)-1-((1-methyl-1H-pyrazol-3-yl)sulfonyl)piperidine-4-carboxamide;
1-(2-(Cyclopropanesulfonamido)pyrimidin-4-yl)-N-(5-(6-cyclopropylpyrazin-2-yl)pyridin-2-yl)-4-methoxycyclohexane-1-carboxamide (diastereomer 1);
1-(2-(Cyclopropanesulfonamido)pyrimidin-4-yl)-N-(5-(6-cyclopropylpyrazin-2-yl)pyridin-2-yl)-4-methoxycyclohexane-1-carboxamide (diastereomer 2);
1-(2-(Cyclopropanesulfonamido)pyrimidin-4-yl)-N-(5-(6-ethoxypyrazin-2-yl)pyridin-2-yl)-4-(pyrrolidin-1-yl)cyclohexane-1-carboxamide (diastereomer 1);
1-(2-(Cydopropanesulfonamido)pyrimidin-4-yl)-N-(5-(6-ethoxypyrazin-2-yl)pyridin-2-yl)-4-(pyrrolidin-1-yl)cyclohexane-1-carboxamide (diastereomer 2);
4-amino-1-(2-(cydopropanesulfonamido)pyrimidin-4-yl)-N-(5-(6-ethoxypyrazin-2-yl)pyridin-2-yl)cyclohexane-1-carboxamide (diastereomer 1);
1-(2-(cydopropanesulfonamido)pyrimidin-4-yl)-N-(5-(6-ethoxypyrazin-2-yl)pyridin-2-yl)-4-morpholinocyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-morpholinocyclohexane-1-carboxamide (diastereomer 2);
1-(2-(cyclopropanesulfonamido)pyrimidin-4-yl)-N-(5-(6-ethoxypyrazin-2-yl)pyridin-2-yl)-4-(methyl(oxetan-3-yl)amino)cyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-((2-methoxyethyl)(methyl)amino)cyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-((2-methoxyethyl)(methyl)amino)cyclohexane-1-carboxamide (diastereomer 2);
1-(2-(cyclopropanesulfonamido)pyrimidin-4-yl)-4-((2,2-difluoroethyl)(methyl)amino)-N-(5-(6-ethoxypyrazin-2-yl)pyridin-2-yl)cyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-(4-methylpiperazin-1-yl)cyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-(4-methylpiperazin-1-yl)cyclohexane-1-carboxamide (diastereomer 2);
4-(6-(cyclopropanesulfonamido)pyrazin-2-yl)-N-(5-(6-ethoxypyrazin-2-yl)pyridin-2-yl)-1-(methylsulfonyl)piperidine-4-carboxamide;
4-(4-(cyclopropanesulfonamido)pyrimidin-2-yl)-N-(5-(6-cyclopropylpyrazin-2-yl)pyridin-2-yl)-1-(methylsulfonyl)piperidine-4-carboxamide;
4-(4-(cyclopropanesulfonamido)pyrimidin-2-yl)-N-(5-(6-ethoxypyrazin-2-yl)pyridin-2-yl)-1-(methylsulfonyl)piperidine-4-carboxamide;
4-(4-(cyclopropanesulfonamido)pyrimidin-2-yl)-N-(5-(6-cyclopropylpyrazin-2-yl)pyridin-2-yl)-1-(ethylsulfonyl)piperidine-4-carboxamide; and
4-(4-(cyclopropanesulfonamido)pyrimidin-2-yl)-N-(5-(6-ethoxypyrazin-2-yl)pyridin-2-yl)-1-(ethylsulfonyl)piperidine-4-carboxamide.

Compounds of Formula (I-b)

The invention provides a compound of formula (I-b):

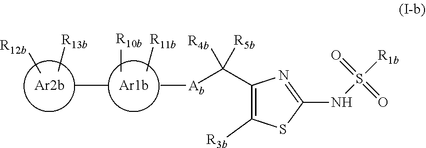

wherein
A_{b} is A_{ab} or A_{bb};
   A_{ab} is —NR_{6b}CH₂— or —NR_{6b}—;
   A_{bb} is —NR_{6b}C(═O)—;
R_{1b} is R_{1ab} or R_{1bb};
wherein:
   R_{1ab} is NR₃₂ₐR_{33b};
R_{1bb} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R_{3b} is H, halo, CH₃, OC₁₋₂alkyl or CF₃;
   or R_{3b} together with R_{5bb} forms a 5- or 6-membered cycloalkyl or 5 or 6 membered oxygen-containing heterocycloalkyl;
R_{4b} and R_{5b} are either R_{4ab} and R_{5ab} or R_{4bb} and R_{5bb};
wherein:
   R_{4ab} and R_{5ab} together with the carbon atom to which they are attached form a C₃₋₆cycloalkyl which is:
      substituted by one or two substituents, each substituent being independently selected from the group consisting of C₁₋₃alkyl, oxo, OH, C₁₋₃ alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21b}R_{22b}; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ab} and R_{5ab} together with the carbon atom to which they are attached may be substituted by one or two substituents, each substituent being independently selected from the group consisting of C₁₋₃ alkyl or OC₁₋₃alkyl; or
   R_{4ab} and R_{5ab} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heteroycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl; or
   R_{4ab} and R_{5ab} together with the carbon atom to which they are attached form a C₃₋₆heterocycloalkyl comprising one nitrogen atom, wherein said nitrogen atom is substituted by —S(O)₂R_{29b}; or
   R_{4bb} and R_{5bb} are each independently H, halo, C₁₋₆alkyl, C₀₋₂alkyleneC₃₋₆cycloalkyl, C₀₋₂alkyleneC₃₋₆heterocycloalkyl, OC₁₋₆alkyl, OC₀₋₂alkyleneC₃₋₆cycloalkyl, OC₀₋₂alkyleneC₃₋₆heterocycloalkyl, C₁₋₃alkyleneOC₁₋₃alkyl, C₁₋₆alkylOH, C₁₋₆haloalkyl, OC₁₋₆haloalkyl or NR_{21b}R_{22b},
      or R_{4bb} is H and R_{5bb} together with R_{3b} form a 5- or 6-membered cycloalkyl or 5 or 6 membered oxygen-containing heterocycloalkyl,
      or R_{4bb} and R_{5bb} together with the carbon atom to which they are attached form a C₃₋₆cycloalkyl or C₃₋₆heterocycloalkyl,
      or R_{4bb} is H and R_{5bb} and R_{6b} are a C₂₋₃alkylene chain forming a 5- or 6-membered ring;
      or R_{4bb} is O and R_{5bb} is absent;
R_{6b} is H or C₁₋₃alkyl,
   or R_{6b} together with R_{11b} when in the ortho-position to group A_{b} are a C₂alkylene chain forming a 5-membered ring,
   or R_{5bb} and R_{6b} are a C₂₋₃alkylene chain forming a 5- or 6-membered ring and R_{4bb} is H;
Ar1b is 6-membered aryl or heteroaryl;
Ar2b is a 6-membered aryl or heteroaryl and is attached to Ar1b in the para position relative to group A_{b};
R_{10b} is H, halo, C₁₋₃alkyl, OC₁₋₂alkyl, C₁₋₂haloalkyl, OC₁₋₂haloalkyl or CN;
R_{11b} is H, F, Cl, CH₃, ethyl, OCH₃, CF₃, OCF₃ or CN,
   or R_{11b}, when in the ortho-position to group A_{b}, together with R_{6b} are a C₂alkylene chain forming a 5-membered ring;
R_{12b} is attached to Ar2b in the ortho or meta position relative to Ar1b and R_{12b} is H, halo, C₁₋₄alkyl, C₂₋₄alkynyl, C₀₋₂alkyleneC₃₋₅cycloalkyl, OC₁₋₄alkyl, OC₀₋₂alkyleneC₃₋₅cycloalkyl, OCH₂CH₂N(CH₃)₂, OH, C₁₋₄alkylOH, CN, C₁₋₃alkyleneOC₁₋₃alkyl, C₁₋₄haloalkyl, OC₁₋₄ haloalkyl, C(═O)C₁₋₂alkyl, NR_{23b}R_{24b}, SO₂C₁₋₄alkyl, SOC₁₋₄alkyl, SC₁₋₄alkyl, SH, C(O)N(CH₃)₂, NHC(O)C₁₋₃alkyl, C₃₋₆heterocycloalkyl comprising one nitrogen located at the point of attachment to Ar2b, or R_{12b} together with a nitrogen atom to which it is attached forms an N-oxide (N⁺—O⁻);
R_{13b} is H, halo, CH₃ or OCH₃;
R_{21b} is H, C₁₋₅alkyl, C(O)C₁₋₅alkyl, C(O)OC₁₋₅alkyl, C₁₋₅alkylOC₁₋₂alkyl, C₁₋₄haloalkyl, or C₄₋₆heterocycloalkyl;
R_{22b} is H or CH₃;
R_{23b} is H or C₁₋₂alkyl;
R_{24b} is H or C₁₋₂alkyl;
R_{29b} is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl; and
R_{32b} is C₁₋₃alkyl and R_{33b} is C₁₋₃alkyl; or
R_{32b} and R_{33b} together with the nitrogen atom to which they are attached form a C₃₋₅heterocycloalkyl;
or a salt and/or solvate thereof and/or derivative thereof.

Suitably, R_{1b} is R₁ₐ; and/or R_{4b} and R_{5b} are R_{4ab} and R_{5ab}; and/or A is A_{ab}.

The invention also provides a compound of formula (I-b):

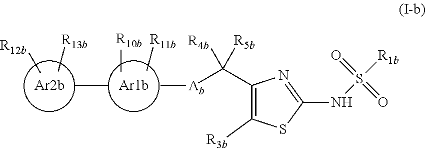

wherein
A_{b} is A_{ab} or A_{bb};
   A_{ab} is —NR_{6b}CH₂— or —NR_{6b}—;
   A_{bb} is —NR_{6b}C(═O)—;
R_{1b} is R_{1ab} or R_{1bb};
wherein:
   R_{1ab} is NR₃₂ₐR_{33b};
R_{1bb} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R_{3b} is H, halo, CH₃, OC₁₋₂alkyl or CF₃;
   or R_{3b} together with R_{5bb} forms a 5- or 6-membered cycloalkyl or 5 or 6 membered oxygen-containing heterocycloalkyl;
R_{4b} and R_{5b} are either R_{4ab} and R_{5ab} or R_{4bb} and R_{5bb};
wherein:
   R_{4ab} and R_{5ab} together with the carbon atom to which they are attached form a C₃₋₆cycloalkyl which is:
      substituted by one or two substituents, each substituent being independently selected from the group consisting of C₁₋₃alkyl, oxo, OH, C₁₋₃ alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21b}R_{22b}; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ab} and R_{5ab} together with the carbon atom to which they are attached may be substituted by one or two substituents, each substituent being independently selected from the group consisting of C₁₋₃ alkyl or OC₁₋₃alkyl; or
   R_{4ab} and R_{5ab} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heteroycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl; or
   R_{4ab} and R_{5ab} together with the carbon atom to which they are attached form a C₃₋₆heterocycloalkyl comprising one nitrogen atom, wherein said nitrogen atom is substituted by —S(O)₂R_{29b}; or
   R_{4bb} and R_{5bb} are each independently H, halo, C₁₋₆alkyl, C₀₋₂alkyleneC₃₋₆cycloalkyl, C₀₋₂alkyleneC₃₋₆heterocycloalkyl, OC₁₋₆alkyl, OC₀₋₂alkyleneC₃₋₆cycloalkyl, OC₀₋₂alkyleneC₃₋₆heterocycloalkyl, C₁₋₃alkyleneOC₁₋₃alkyl, C₁₋₆alkylOH, C₁₋₆haloalkyl, OC₁₋₆haloalkyl or NR_{21b}R_{22b},
      or R_{4bb} is H and R_{5bb} together with R_{3b} form a 5- or 6-membered cycloalkyl or 5 or 6 membered oxygen-containing heterocycloalkyl,
      or R_{4bb} and R_{5bb} together with the carbon atom to which they are attached form a C₃₋₆cycloalkyl or C₃₋₆heterocycloalkyl,
      or R_{4bb} is H and R_{5bb} and R_{6b} are a C₂₋₃alkylene chain forming a 5- or 6-membered ring;
      or R_{4bb} is O and R_{5bb} is absent;
R_{6b} is H or C₁₋₃alkyl,
   or R_{6b} together with R_{11b} when in the ortho-position to group A_{b} are a C₂alkylene chain forming a 5-membered ring,
   or R_{5bb} and R_{6b} are a C₂₋₃alkylene chain forming a 5- or 6-membered ring and R_{4bb} is H;
Ar1b is 6-membered aryl or heteroaryl;
Ar2b is a 6-membered aryl or heteroaryl and is attached to Ar1b in the pare position relative to group A_{b};
R_{10b} is H, halo, C₁₋₃alkyl, OC₁₋₂alkyl, C₁₋₂haloalkyl, OC₁₋₂haloalkyl or CN;
R_{11b} is H, F, Cl, CH₃, ethyl, OCH₃, CF₃, OCF₃ or CN,
   or R_{11b}, when in the ortho-position to group A_{b}, together with R_{6b} are a C₂alkylene chain forming a 5-membered ring;
R₁₂ₐ is attached to Ar2b in the ortho or meta position relative to Ar1b and R₁₂ₐ is H, halo, C₁₋₄alkyl, C₂₋₄alkynyl, C₀₋₂alkyleneC₃₋₅cycloalkyl, OC₁₋₄alkyl, OC₀₋₂alkyleneC₃₋₅cycloalkyl, OCH₂CH₂N(CH₃)₂, OH, C₁₋₄alkylOH, CN, C₁₋₃alkyleneOC₁₋₃alkyl, C₁₋₄haloalkyl, OC₁₋₄ haloalkyl, C(═O)C₁₋₂alkyl, NR_{23b}R_{24b}, SO₂C₁₋₄alkyl, SOC₁₋₄alkyl, SC₁₋₄alkyl, SH, C(O)N(CH₃)₂, NHC(O)C₁₋₃alkyl, C₃₋₆heterocycloalkyl comprising one nitrogen located at the point of attachment to Ar2b, or R₁₂ₐ together with a nitrogen atom to which it is attached forms an N-oxide (N⁺—O⁻);
R_{13b} is H, halo, CH₃ or OCH₃;
R_{21b} is H, C₁₋₅alkyl, C(O)C₁₋₅alkyl, C(O)OC₁₋₅alkyl;
R_{22b} is H or CH₃;
R_{23b} is H or C₁₋₂alkyl;
R_{24b} is H or C₁₋₂alky;
R_{29b} is C₁₋₃alkyl, C₀₋₂alkyleneC₃₋₅cycloalkyl which cycloalkyl is optionally substituted by CH₃, or CF₃; and
R₃₂ₐ is C₁₋₃alkyl and R_{33b} is C₁₋₃alkyl; or
R_{32b} and R_{33b} together with the nitrogen atom to which they are attached form a C₃₋₅heterocycloalkyl;
or a salt and/or solvate thereof and/or derivative thereof.

Suitably, R_{1b} is R_{1ab}; and/or R_{4b} and R_{5b} are R_{4ab} and R_{5ab}; and/or A is A_{ab}.

In one embodiment of the invention, R_{1b} is R_{1ab}, i.e. is NR_{32b}R_{33b}. In an embodiment, R_{32b} is C₁₋₃ alkyl, such as methyl or ethyl, e.g. methyl. In an embodiment, R_{33b} is C₁₋₃alkyl, such as methyl or ethyl, e.g. methyl. Suitably, R_{32b} and R_{33b} are both methyl. Suitably, R₃₂ₐ and R_{33b} are both ethyl. Suitably, R_{32b} is methyl and R_{33b} is ethyl.

In another embodiment, R_{32b} and R_{33b} together with the nitrogen atom to which they are attached form a C₃₋₅heterocycloalkyl. Suitably, the C₃₋₅heterocycloalkyl is aziridinyl, azetidinyl or pyrrolidinyl.

Suitably, R_{1b} is R_{1bb}.

In one embodiment of the invention R_{1bb} is C₁₋₅alkyl. When R_{1bb} is C₁₋₅alkyl, R_{1bb} may be methyl, ethyl, propyl (n-propyl or isopropyl), butyl (n-butyl, isobutyl, sec-butyl or tert-butyl) or pentyl (e.g. n-pentyl, sec-pentyl or 3-pentyl). Suitably, when R_{1bb} is C₁₋₅alkyl, R_{1bb} may be methyl, ethyl, propyl (e.g. isopropyl) or butyl (e.g. sec-butyl or tert-butyl), especially methyl, ethyl or isopropyl and in particular methyl.

In a second embodiment of the invention R_{1bb} is C₀₋₂alkyleneC₃₋₅cycloalkyl which cycloalkyl is optionally substituted by CH₃. In some embodiments, R_{1bb} is C₀₋₂alkyleneC₃₋₅cycloalkyl. In other embodiments, R_{1bb} is C₀₋₂alkyleneC₃₋₅cycloalkyl which cycloalkyl is substituted by CH₃. R_{1bb} may be C₃₋₅cycloalkyl, which cycloalkyl is optionally substituted by CH₃. R_{1bb} may be C₁alkyleneC₃₋₅cycloalkyl, which cycloalkyl is optionally substituted by CH₃. R_{1bb} may be C₂alkyleneC₃₋₅cycloalkyl, which cycloalkyl is optionally substituted by CH₃. R_{1bb} may be C₀₋₂alkyleneC₃cycloalkyl, which cycloalkyl is optionally substituted by CH₃. R_{1bb} may be C₀₋₂alkyleneC₄cycloalkyl, which cycloalkyl is optionally substituted by CH₃. R_{1bb} may be C₀₋₂alkyleneC₅cycloalkyl, which cycloalkyl is optionally substituted by CH₃. Suitably, where C₀₋₂alkyleneC₃₋₅cycloalkyl is optionally substituted by CH₃, the CH₃ is at the point of attachment of the C₃₋₅cycloalkyl to the C₀₋₂alkylene.

In a third embodiment of the invention R_{1bb} is CF₃.

In a fourth embodiment of the invention R_{1bb} is C₁₋₃alkyleneOC₁₋₂alkyl such as C₁₋₂alkyleneOC₁₋₂alkyl. When R_{1bb} is C₁₋₃alkyleneOC₁₋₂alkyl, R_{1bb} may be methoxymethyl, methoxyethyl, ethoxymethyl, ethoxyethyl, propoxymethyl or propoxyethyl. When R_{1bb} is C₁₋₂alkyleneOC₁₋₂alkyl, R_{1bb} may be methoxymethyl, methoxyethyl, ethoxymethyl or ethoxyethyl.

Suitably R_{1bb} is cyclopropyl, cyclopropyl substituted by CH₃ at the point of attachment, cyclobutyl, CH₃, isopropyl, sec-butyl, tert-butyl or CF₃. In particular R_{1bb} is cyclopropyl, cyclopropyl substituted by CH₃ at the point of attachment, cyclobutyl, CH₃, isopropyl, sec-butyl or tert-butyl, especially cyclopropyl, cyclopropyl substituted by CH₃ at the point of attachment, cyclobutyl or isopropyl, such as cyclopropyl or cyclopropyl substituted by CH₃ at the point of attachment.

Additionally of interest is when R_{1bb} is cyclopentyl, methyl, ethyl, cyclopropylmethylene and methoxyethyl, in particular cyclopentyl, methyl, ethyl and cyclopropylmethylene, especially ethyl and methyl, such as methyl.

Consequently, suitably R_{1bb} is cyclopropyl, cyclopropyl substituted by CH₃ at the point of attachment, cyclopropylmethylene, cyclobutyl, cyclopentyl, CH₃, ethyl, isopropyl, sec-butyl, tert-butyl, methoxyethyl or CF₃. In particular R_{1bb} is cyclopropyl, cyclopropyl substituted by CH₃ at the point of attachment, cyclopropylmethylene, cyclobutyl, cyclopentyl, CH₃, ethyl, isopropyl, sec-butyl or tert-butyl, especially cyclopropyl, cyclopropyl substituted by CH₃ at the point of attachment, cyclobutyl, CH₃, ethyl or isopropyl, such as cyclopropyl, cyclopropyl substituted by CH₃ at the point of attachment, ethyl or methyl such as cyclopropyl, cyclopropyl substituted by CH₃ at the point of attachment or methyl.

In one embodiment R_{3b} is H. In a second embodiment R_{3b} is halo, in particular chloro or fluoro, especially chloro. In a third embodiment R_{3b} is CH₃. In a fourth embodiment R_{3b} is CF₃. In a fifth embodiment R_{3b} together with R_{5bb} forms a 5- or 6-membered cycloalkyl, in particular a 5-membered cycloalkyl. In a sixth embodiment R_{3b} is OC₁₋₂alkyl such as OCH₃. In a seventh embodiment R_{3b} together with R_{5bb} forms a 5- or 6-membered oxygen-containing heterocycloalkyl, in particular a 5-membered heterocycloalkyl.

The phrase ‘R_{3b} together with R_{5bb} forms a 5- or 6-membered cycloalkyl’ means that compounds with the following exemplary substructure are formed:

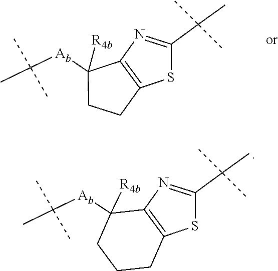

The phrase ‘R_{3b} together with R_{5bb} forms a 5- or 6-membered oxygen containing heterocycloalkyl’ means that compounds with the following substructure are formed:

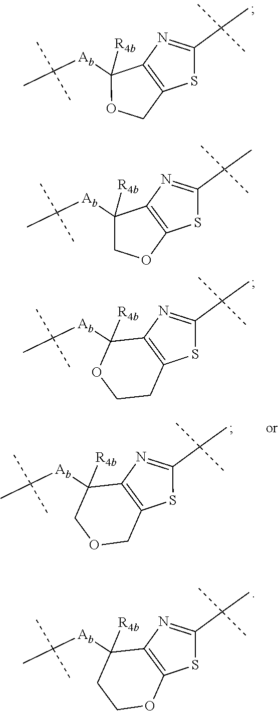

In particular R_{3b} is H, CH₃ or R_{3b} together with R_{5bb} forms a 5- or 6-membered cycloalkyl, especially H, CH₃ or R_{3b} together with R_{5bb} forms a 5-membered cycloalkyl, such as R_{3b} is H or CH₃, e.g. H.

In one embodiment, R_{4b} and R_{5b} are R_{4ab} and R_{5ab}.

Suitably, R_{4ab} and R_{5ab} together with the carbon atom to which they are attached form a C₃₋₆cycloalkyl which is:
substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21b}R₂₂ₐ.

In one embodiment, the C₃₋₆cycloalkyl is cyclopropyl. In another embodiment, the C₃₋₆cycloalkyl is cyclobutyl. In another embodiment, the C₃₋₆cycloalkyl is cyclopentyl. In another embodiment, the C₃₋₆cycloalkyl is cyclohexyl.

In one embodiment the C₃₋₆cycloalkyl is substituted by one substituent. In a second embodiment the C₃₋₆cycloalkyl is substituted by two substituents.

In one embodiment, the substituent is C₁₋₃alkyl. Suitably, the substituent is methyl. Suitably, the substituent is ethyl. Suitably, the substituent is n-propyl. Suitably, the substituent is iso-propyl.

In a second embodiment, the substituent is C₁₋₂alkylOH. Suitably, the substituent is CH₂OH. Suitably, the substituent is CH₂CH₂OH. Suitably, the substituent is CH₂CH₂CH₂OH.

In a third embodiment, the substituent is C₁₋₃haloalkyl. Suitably the C₁₋₃alkyl group is substituted by one two or three, such as one, halogen atom. Suitably, the halogen atom is fluoro or chloro such as fluoro. Suitably, the substituent is C₁haloalkyl such as CF₃. Suitably, the substituent is C₂haloalkyl such as CH₂CF₃.

In a fourth embodiment, the substituent is C₀₋₂alkyleneC₃₋₆cycloalkyl, in particular C₀₋₂alkyleneC₃₋₅cycloalkyl, such as C₃₋₅cycloalkyl, C₁alkyleneC₃₋₅cycloalkyl or C₂alkyleneC₃₋₅cycloalkyl.

In a fifth embodiment, the substituent is C₀₋₂alkyleneC₃₋₆heterocycloalkyl such as C₀₋₂alkyleneC₃heterocycloalkyl, C₀₋₂alkyleneC₄heterocycloalkyl, C₀₋₂alkyleneC₅heterocycloalkyl, C₀₋₂alkyleneC₆heterocycloalkyl, C₀alkyleneC₃₋₆heterocycloalkyl, C₁alkyleneC₃₋₆heterocycloalkyl and C₂alkyleneC₃₋₆heterocycloalkyl. Suitably the heterocycloalkyl is a heterocyclopropyl, heterocyclobutyl, heterocyclopentyl or heterocyclohexyl ring such as a heterocyclohexyl ring. Suitably, the heterocyclopentyl ring is tetrahydrofuranyl or pyrrolidinyl. Suitably, the heterocyclohexyl ring is tetrahydropyranyl or piperidinyl. Any nitrogen atom(s) in the C₃₋₆heterocycloalkyl ring may be substituted (such as one nitrogen atom is substituted), for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃ alkylOC₁₋₂alkyl such as C(O)CH₂OCH₃, and C₁₋₂alkylC(O)OC₁₋₄alkyl such as CH₂C(O)OCH₂CH₃. Suitably, any nitrogen atom in the C₃₋₆heterocycloalkyl ring is not substituted.

In a sixth embodiment, the substituent is C₁₋₃alkyleneOC₁₋₃alkyl, in particular C₁₋₂alkyleneOC₁₋₂alkyl such as C₁alkyleneOC₁alkyl, C₂alkyleneOC₁alkyl, C₁alkyleneOC₂alkyl or C₂alkyleneOC₂alkyl.

In a seventh embodiment, the substituent is halo, in particular fluoro or chloro such as chloro.

In an eighth embodiment, the substituent is OC₁₋₃haloalkyl. Suitably the OC₁₋₃alkyl group is substituted by one two or three, such as one, halogen atom. Suitably, the halogen atom is fluoro or chloro such as fluoro. Suitably, the substituent is OC₁haloalkyl such as OCF₃. Suitably, the substituent is OC₂haloalkyl such as OCH₂CF₃.

In a ninth embodiment, the substituent is OC₀₋₂alkyleneC₃₋₆cycloalkyl, such as OC₃₋₆cycloalkyl, OC₁alkyleneC₃₋₆cycloalkyl or OC₂alkyleneC₃₋₆cycloalkyl.

In a tenth embodiment, the substituent is OC₀₋₂alkyleneC₃₋₆heterocycloalkyl such as OC₀₋₂alkyleneC₃heterocycloalkyl, OC₀₋₂alkyleneC₄heterocycloalkyl, OC₀₋₂alkyleneC₅heterocycloalkyl, OC₀₋₂alkyleneC₆heterocycloalkyl, OC₀alkyleneC₃₋₆heterocycloalkyl, OC₁alkyleneC₃₋₆heterocycloalkyl and OC₂alkyleneC₃₋₆heterocycloalkyl. Suitably the heterocycloalkyl is a heterocyclopropyl, heterocyclobutyl, heterocyclopentyl or heterocyclohexyl ring such as a heterocyclohexyl ring. Suitably, the heterocyclopentyl ring is tetrahydrofuranyl or pyrrolidinyl. Suitably, the heterocyclohexyl ring is tetrahydropyranyl or piperidinyl. Any nitrogen atom(s) (such as one nitrogen atom) in the C₃₋₆heterocycloalkyl ring may be substituted, for example by C₁₋₄ alkyl, C(O)H, C(O)C₁₋₄alkyl, C(O)OC₁₋₄alkyl, C(O)OC₁₋₄alkylaryl such as C(O)OBz, C(O)NHC₁₋₄ alkyl, C(O)NHC₁₋₄alkylaryl such as C(O)NHBz, an Fmoc group, C(O)C₁₋₄haloalkyl, C(O)OC₁₋₄ haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃ alkylOC₁₋₂alkyl such as C(O)CH₂OCH₃, and C₁₋₂alkylC(O)OC₄alkyl such as CH₂C(O)OCH₂CH₃. Suitably, any nitrogen atom in the C₃₋₆heterocycloalkyl ring is not substituted.

In an eleventh embodiment, the substituent is OC₁₋₃alkyl, such as OCH₃ or OCH₂CH₃.

In a twelfth embodiment, the substituent is NR_{21b}R_{22b}.

In one embodiment R_{21b} is H. In a second embodiment R_{21b} is C₁₋₅alkyl, such as methyl, ethyl or propyl, especially methyl. In a third embodiment R_{21b} is C(O)C₁₋₅alkyl, such as C(O)CH₃. In a fourth embodiment R_{21b} is C(O)OC₁₋₅alkyl, such as C(O)OCH₃ or C(O)Otert-butyl. In a fifth embodiment R_{21b} is C₁₋₃alkylOC₁₋₂alkyl such as C₁alkylOC₁alkyl, C₂alkylOC₁alkyl or C₃alkylOC₁alkyl e.g. C₂alkylOC₁alkyl. In a sixth embodiment, R_{21b} is C₁₋₄haloalkyl, such as CF₃, CH₂CF₃ or CH₂CHF₂ e.g. CH₂CHF₂. In a seventh embodiment R_{21b} is C₄₋₆heterocycloalkyl, such as oxetanyl, tetrahydrofuranyl or tetrahydropyranyl e.g. oxetanyl, in particular 3-oxetanyl.

When the substituent is NR_{21b}R_{22b}, in one embodiment R_{22b} is H. In a second embodiment R_{22b} is methyl.

Suitably, R_{21b} is C(O)OCH₃ and R_{22b} is H. Suitably, R_{21b} is C(O)CH₃ and R_{22b} is H. Suitably, R_{21b} and R_{22b} are both CH₃. Suitably, R_{21b} and R_{22b} are both H.

In a thirteenth embodiment, the substituent is oxo.

In a fourteenth embodiment, the substituent is OH.

Suitably, the one or two substituents, in particular one substituent, are independently selected from the group consisting of C₁₋₃alkyl, oxo, OH, C₁₋₃alkylOH, C₁₋₃haloalkyl, halo, OC₁₋₃haloalkyl, OC₁₋₃alkyl and NR_{21b}R_{22b}.

More suitably, the substituent is independently selected from the group consisting of oxo, OH, halo, OC₁₋₃alkyl and NR_{21b}R_{22b}.

Most suitably, the substituent is independently selected from the group consisting of oxo, OH, fluoro, NR_{21b}R_{22b}.

Alternatively, R_{4ab} and R_{5ab} together with the carbon atom to which they are attached form a C₃₋₆cycloalkyl and one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl.

In one embodiment the C₃₋₆cycloalkyl formed by R_{4ab} and R_{5ab} together with the carbon atom to which they are attached is unsubstituted. In a second embodiment the C₃₋₆cycloalkyl formed by R_{4ab} and R_{5ab} together with the carbon atom to which they are attached is substituted by one or two substituents, in particular one substituent. Suitably, each substituent is independently selected from the group consisting of C₁₋₂alkyl or OCH₃.

Suitably one of the carbons of the C₃₋₆cycloalkyl which is formed by R_{4ab} and R_{5ab} is a spiro centre such that a spirocyclic ring system is formed, wherein the C₃₋₆cycloalkyl which is formed by R_{4ab} and R_{5ab} is a C₄₋₆cycloalkyl. Suitably the C₃₋₅heterocycloalkyl is an oxygen containing C₃₋₆heterocycloalkyl. Suitably, the C₃₋₆heterocycloalkyl is an oxygen comprising, such as containing, C₃₋₆heterocycloalkyl ring, such as a C₅cycloalkyl ring.

In an embodiment, R_{4ab} and R_{5ab} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heteroycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl. Suitably, each substituent is independently selected from the group consisting of C₁₋₂alkyl or OCH₃.

Suitably one of the carbons of the C₃₋₆heterocycloalkyl is a spiro centre such that a spirocyclic ring system is formed containing further ring C, wherein C is a C₄₋₆heterocycloalkyl. Suitably the C₄₋₆heterocycloalkyl is an oxygen containing C₄₋₆heterocycloalkyl such as tetrahydropyranyl or 1,3-dioxolanyl.

In an embodiment, R_{4ab} and R_{5ab} together with the carbon atom to which they are attached form a C₃₋₆heterocycloalkyl comprising one nitrogen atom, wherein said nitrogen atom is substituted by —S(O)₂R_{29b}.

Suitably, the C₃₋₆heterocycloalkyl is selected from the group consisting of aziridinyl, azetidinyl, pyrrolidinyl and piperidinyl such as piperidinyl.

Suitably, when the C₃₋₆heterocycloalkyl is piperidinyl, the nitrogen atom is in the 4-position relative to the quaternary carbon:

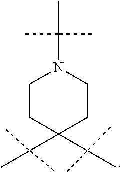

The C₃₋₆heterocycloalkyl may be other groups as defined elsewhere herein.

In an embodiment, R_{29b} is C₁₋₃alkyl, C₀₋₂alkyleneC₃₋₅cycloalkyl which cycloalkyl is optionally substituted by CH₃, or CF₃. In one embodiment, R_{29b} is C₁₋₃alkyl such as methyl. In another embodiment, R_{29b} is C₀₋₂alkyleneC₃₋₅cycloalkyl which cycloalkyl is optionally substituted by CH₃. In some embodiments, R₂ is C₀₋₂alkyleneC₃₋₅cycloalkyl. In other embodiments, R₂₉ₐ is C₀₋₂alkyleneC₃₋₅cycloalkyl which cycloalkyl is substituted by CH₃. R_{29b} may be C₃₋₅cycloalkyl, which cycloalkyl is optionally substituted by CH₃. R_{29b} may be C₁alkyleneC₃₋₅cycloalkyl, which cycloalkyl is optionally substituted by CH₃. R_{29b} may be C₂alkyleneC₃₋₅cycloalkyl, which cycloalkyl is optionally substituted by CH₃. R_{29b} may be C₀₋₂alkyleneC₃cycloalkyl, which cycloalkyl is optionally substituted by CH₃. R_{29b} may be C₀₋₂alkyleneC₄cycloalkyl, which cycloalkyl is optionally substituted by CH₃. R_{29b} may be C₀₋₂alkyleneC₅cycloalkyl, which cycloalkyl is optionally substituted by CH₃. Suitably, where C₀₋₂alkyleneC₃₋₅cycloalkyl is optionally substituted by CH₃, the CH₃ is at the point of attachment of the C₃₋₅cycloalkyl to the C₀₋₂alkylene. In another embodiment, R_{29b} is CF₃. In another embodiment, R_{29b} is N(C₁₋₃alkyl)₂ such as N(CH₃)₂. In another embodiment, R_{29b} is a 5 or 6 membered heteroaryl wherein the 5 or 6 membered heteroaryl is optionally substituted by methyl. In one embodiment, the 5 or 6 membered heteroaryl is substituted by methyl. In one embodiment, the 5 or 6 membered heteroaryl is not substituted by methyl. In one embodiment, R_{29b} is a 5-membered heteroaryl such as pyrrolyl, furanyl, thiophenyl, imidazolyl, pyrazolyl, oxazolyl, isoxazolyl, thiazolyl, isothiazolyl, such as pyrazolyl. Suitably the pyrazolyl is substituted by methyl. In another embodiment, R_{29b} is a 6-membered heteroaryl such as pyridinyl, pyridazinyl, pyrimidinyl or pyrazinyl.

In another embodiment, R_{4b} and R_{5b} are R_{4bb} and R_{5bb}.

In one embodiment R_{4bb} is O and R_{5bb} is absent. The person skilled in the art will appreciate that in this embodiment, the following moiety forms, in order to retain the correct carbon valency of 4:

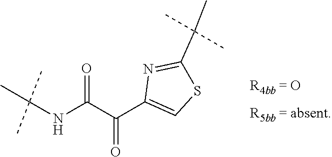

In a second embodiment, R_{4bb} and R_{5bb} together with the carbon atom to which they are attached form a C₃₋₆cycloalkyl, such as cyclopropyl, cyclobutyl or cyclopentyl. In a third embodiment R_{4bb} is C₁₋₆alkyl, in particular C₁₋₄alkyl such as methyl, ethyl, propyl (n-propyl or isopropyl) or butyl (n-butyl, isobutyl, sec-butyl or tert-butyl). In a fourth embodiment R_{4bb} is C₀₋₂alkyleneC₃₋₆cycloalkyl, in particular C₀₋₂alkyleneC₃₋₅cycloalkyl, such as C₃₋₅cycloalkyl, C₁alkyleneC₃₋₅cycloalkyl or C₂alkyleneC₃₋₅cycloalkyl. In a fifth embodiment R_{4bb} is OC₁₋₆alkyl, in particular OC₁₋₄alkyl, such as methoxy, ethoxy, propoxy (n-propoxy or isopropoxy) or butoxy (n-butoxy, isobutoxy, sec-butoxy or tert-butoxy). In a sixth embodiment R_{4bb} is OC₀₋₂alkyleneC₃₋₆cycloalkyl, such as OC₃₋₆cycloalkyl, OC₁alkyleneC₃₋₆cycloalkyl or OC₂alkyleneC₃₋₆cycloalkyl. In a seventh embodiment R_{4bb} is C₁₋₃alkyleneOC₁₋₃alkyl, in particular C₁₋₂alkyleneOC₁₋₂alkyl such as C₁alkyleneOC₁alkyl, C₂alkyleneOC₁alkyl, C₁alkyleneOC₂alkyl or C₂alkyleneOC₂alkyl. In an eighth embodiment R_{4bb} is C₁₋₆haloalkyl, in particular C₁₋₄₄haloalkyl. In a ninth embodiment R_{4bb} is OC₁₋₆haloalkyl, in particular OC₁₋₄haloalkyl. In a tenth embodiment R_{4bb} is H. In an eleventh embodiment R_{4bb} is halo such as fluoro. In a twelfth embodiment R_{4bb} is C₁₋₆alkylOH, such as CH₂OH or CH₂CH₂OH, in particular CH₂CH₂OH. In a thirteenth embodiment R_{4bb} is NR_{21b}R_{22b}. In a fourteenth embodiment, R_{4bb} is C₀₋₂alkyleneC₃₋₆heterocycloalkyl such as C₀₋₂alkyleneC₃heterocycloalkyl, C₀₋₂alkyleneC₄heterocycloalkyl, C₀₋₂alkyleneC₅heterocycloalkyl, C₀₋₂alkyleneC₆heterocycloalkyl, C₀alkyleneC₃₋₆heterocycloalkyl, C₁alkyleneC₃₋₆heterocycloalkyl and C₂alkyleneC₃₋₆heterocycloalkyl. Suitably the heterocycloalkyl is a heterocyclopropyl, heterocyclobutyl, heterocyclopentyl or heterocyclohexyl ring such as a heterocyclohexyl ring. Suitably, the heterocyclopentyl ring is tetrahydrofuranyl or pyrrolidinyl. Suitably, the heterocyclohexyl ring is tetrahydropyranyl or piperidinyl. Any nitrogen atom(s) (such as one nitrogen atom) in the C₃₋₆heterocycloalkyl ring may be substituted, for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄ haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃alkylOC₁₋₂alkyl such as C(O)CH₂OCH₃, and C₁₋₂alkylC(O)OC₁₋₄alkyl such as CH₂C(O)OCH₂CH₃. Suitably, any nitrogen atom in the C₃₋₆heterocycloalkyl ring is not substituted. In a fifteenth embodiment, R_{4bb} and R_{5bb} together with the carbon atom to which they are attached form a C₃₋₆heterocycloalkyl, such as tetrahydrofuranyl, tetrahydropyranyl, pyrrolidinyl or piperidinyl, such as tetrahydrofuranyl or piperidinyl. If the C₃₋₆heterocycloalkyl group comprises (e.g. contains) a nitrogen atom, independently the nitrogen atom(s) may be unsubstituted (NH) or the nitrogen atom(s) may be substituted, for example substituted by a group selected from the following: C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃alkylOC₁₋₂alkyl such as C(O)CH₂OCH₃, and C₁₋₂alkylC(O)OC₁₋₄alkyl such as CH₂C(O)OCH₂CH₃. In a fifteenth embodiment, R_{4bb} is H and R_{5bb} and R_{6b} are a C₂₋₃alkylene chain forming a 5- or 6-membered ring. In a sixteenth embodiment, R_{4bb} is OC₀₋₂alkyleneC₃₋₆heterocycloalkyl such as OC₀₋₂alkyleneC₃heterocycloalkyl, OC₀₋₂alkyleneC₄heterocycloalkyl, OC₀₋₂alkyleneC₅heterocycloalkyl, C₀₋₂alkyleneC₆heterocycloalkyl, OC₀alkyleneC₃₋₆heterocycloalkyl, OC₁alkyleneC₃₋₆heterocycloalkyl and OC₂alkyleneC₃₋₆heterocycloalkyl. Suitably the heterocycloalkyl is a heterocyclopropyl, heterocyclobutyl, heterocyclopentyl or heterocyclohexyl ring such as a heterocyclohexyl ring. Suitably, the heterocyclopentyl ring is tetrahydrofuranyl or pyrrolidinyl. Suitably, the heterocyclohexyl ring is tetrahydropyranyl or piperidinyl. Any nitrogen atom(s) (such as one nitrogen atom) in the C₃₋₆heterocycloalkyl ring may be substituted, for example by C₁₋₄ alkyl, C(O)H, C(O)C₁₋₄alkyl, C(O)OC₁₋₄alkyl, C(O)OC₁₋₄alkylaryl such as C(O)OBz, C(O)NHC₁₋₄ alkyl, C(O)NHC₁₋₄alkylaryl such as C(O)NHBz, an Fmoc group, C(O)C₁₋₄haloalkyl, C(O)OC₁₋₄ haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃ alkylOC₁₋₂alkyl such as C(O)CH₂OCH₃, and C₁₋₂alkylC(O)OC₁₋₄alkyl such as CH₂C(O)OCH₂CH₃. Suitably, any nitrogen atom in the C₃₋₆heterocycloalkyl ring is not substituted.

When R_{4bb} is H and R_{5bb} and R_{6b} are a C₂₋₃alkylene chain forming a 5- or 6-membered ring, suitably a 5-membered ring, compounds comprising one of the following moieties are formed:

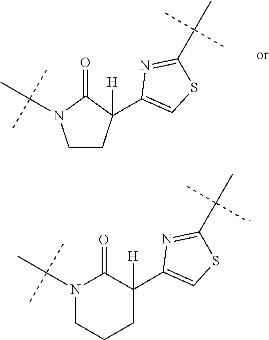

When R_{4bb} is NR_{21b}R_{22b}, in one embodiment R_{21b} is H. In a second embodiment R_{21b} is C₁₋₅alkyl, such as methyl, ethyl or propyl, especially methyl. In a third embodiment R_{21b} is C(O)C₁₋₅alkyl, such as C(O)CH₃. In a fourth embodiment R_{21b} is C(O)OC₁₋₅alkyl, such as C(O)OCH₃ or C(O)Otert-butyl. In a fifth embodiment R_{21b} is C₁₋₃alkylOC₁₋₂alkyl such as C₁alkylOC₁alkyl, C₂alkylOC₁alkyl or C₃alkylOC₁alkyl e.g. C₂alkylOC₁alkyl. In a sixth embodiment, R_{21b} is C₁₋₄ haloalkyl, such as CF₃, CH₂CF₃ or CH₂CHF₂ e.g. CH₂CHF₂. In a seventh embodiment R_{21b} is C₄₋₆heterocycloalkyl, such as oxetanyl, tetrahydrofuranyl or tetrahydropyranyl e.g. oxetanyl, in particular 3-oxetanyl.

When R_{4bb} is NR_{21b}R_{22b}, in one embodiment R_{22b} is H. In a second embodiment R_{22b} is methyl.

Suitably R_{4bb} is H, CH₃, ethyl, isopropyl, fluoro, OCH₃, isopropoxy or CH₂CH₂OCH₃, in particular H, CH₃, ethyl, fluoro, OCH₃, isopropoxy or CH₂CH₂OCH₃, especially H, CH₃, ethyl, OCH₃ or CH₂CH₂OCH₃.

Additionally of interest is when R_{4bb} is NH₂, N(CH₃)₂, NHC(O)CH₃, NHC(O)OCH₃, NHC(O)Otert-butyl and CH₂CH₂OH, especially, N(CH₃)₂, NHC(O)CH₃, NHC(O)OCH₃.

Suitably, R_{21b} is C(O)OCH₃ and R_{22b} is H. Suitably, R_{21b} is C(O)CH₃ and R_{22b} is H. Suitably, R_{21b} and R_{22b} are both CH₃. Suitably, R_{21b} and R_{22b} are both H.

Consequently, suitably R_{4bb} is H, CH₃, ethyl, isopropyl, fluoro, OCH₃, isopropoxy, CH₂CH₂OCH₃, NH₂, N(CH₃)₂, NHC(O)CH₃, NHC(O)OCH₃, NHC(O)Otert-butyl or CH₂CH₂OH, in particular H, CH₃, ethyl, fluoro, OCH₃, isopropoxy, CH₂CH₂OCH₃, NH₂, N(CH₃)₂, NHC(O)CH₃, NHC(O)OCH₃, NHC(O)Otert-butyl or CH₂CH₂OH, especially H, CH₃, ethyl, OCH₃, CH₂CH₂OCH₃, N(CH₃)₂, NHC(O)CH₃ or NHC(O)OCH₃.

Suitably R_{4bb} may be C═O and R_{5bb} is absent.

Suitably R_{4bb} and R_{5bb} together with the carbon atom to which they are attached form a cyclopropyl or cyclopentyl, in particular a cyclopentyl.

Suitably R_{4bb} is H and R_{3b} together with R_{5bb} forms a 5- or 6-membered cycloalkyl, in particular a 5-membered cycloalkyl, especially R_{4bb} is H and R_{3b} together with R_{5bb} forms a 5- or 6-membered cycloalkyl, such as a 5-membered cycloalkyl.

In one embodiment R_{5bb} is C₁₋₆alkyl, in particular C₁₋₄alkyl, such as methyl, ethyl, propyl (n-propyl or isopropyl) or butyl (n-butyl, isobutyl, sec-butyl or tert-butyl). In a second embodiment R_{5bb} is C₀₋₂alkyleneC₃₋₆cycloalkyl, in particular C₀₋₂alkyleneC₃₋₅cycloalkyl, such as C₃₋₅cycloalkyl, C₁alkyleneC₃₋₅cycloalkyl or C₂alkyleneC₃₋₅cycloalkyl. In a third embodiment R_{5bb} is OC₁₋₆alkyl, in particular OC₁₋₄alkyl, such as methoxy, ethoxy, propoxy (n-propoxy or isopropoxy) or butoxy (n-butoxy, isobutoxy, sec-butoxy or tert-butoxy). In a fourth embodiment R_{5bb} is OC₀₋₂alkyleneC₃₋₆cycloalkyl, such as OC₃₋₆cycloalkyl, OC₁alkyleneC₃₋₆cycloalkyl or OC₂alkyleneC₃₋₆cycloalkyl. In a fifth embodiment R_{5bb} is C₁₋₃alkyleneOC₁₋₃alkyl, in particular C₁₋₂alkyleneOC₁₋₂alkyl such as C₁alkyleneOC₁alkyl, C₂alkyleneOC₁alkyl, C₁alkyleneOC₂alkyl or C₂alkyleneOC₂alkyl. In a sixth embodiment R_{5bb} is C₁₋₆haloalkyl, in particular C₁₋₄haloalkyl. In a seventh embodiment R_{5bb} is OC₁₋₆haloalkyl, in particular OC₁₋₄haloalkyl. In an eighth embodiment R_{5bb} is H. In a ninth embodiment R_{5bb} is halo such as fluoro. In a tenth embodiment R_{5bb} is C₁₋₆alkylOH, such as CH₂OH or CH₂CH₂OH, in particular CH₂CH₂OH. In an eleventh embodiment R_{5bb} is NR_{21b}R_{22b}. In a twelfth embodiment, R_{5bb} is C₀₋₂alkyleneC₃₋₆heterocycloalkyl such as C₀₋₂alkyleneC₃₋₆heterocycloalkyl, C₀₋₂alkyleneC₄heterocycloalkyl, C₀₋₂alkyleneC₅heterocycloalkyl, C₀₋₂alkyleneC₆heterocycloalkyl, C₀alkyleneC₃₋₆heterocycloalkyl, C₁alkyleneC₃₋₆heterocycloalkyl and C₂alkyleneC₃₋₆heterocycloalkyl. Suitably the heterocycloalkyl is a heterocyclopropyl, heterocyclobutyl, heterocyclopentyl or heterocyclohexyl ring such as a heterocyclohexyl ring. Suitably, the heterocyclopentyl ring is tetrahydrofuranyl or pyrrolidinyl. Suitably, the heterocyclohexyl ring is tetrahydropyranyl or piperidinyl. Any nitrogen atom(s) in the C₃₋₆heterocycloalkyl ring may be substituted (such as one nitrogen atom is substituted), for example by C₁₋₄alkyl, C(O)H, C(O)C₁₋₄ alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃alkylOC₁₋₂alkyl such as C(O)CH₂OCH₃, and C₁₋₂alkylC(O)OC₁₋₄alkyl such as CH₂C(O)OCH₂CH₃. Suitably, any nitrogen atom in the C₃₋₆heterocycloalkyl ring is not substituted. In a thirteenth embodiment, R_{5bb} is OC₀₋₂alkyleneC₃₋₆heterocycloalkyl such as OC₀₋₂alkyleneC₃heterocycloalkyl, OC₀₋₂alkyleneC₄heterocycloalkyl, OC₀₋₂alkyleneC₅heterocycloalkyl, OC₀₋₂alkyleneC₆heterocycloalkyl, OC₀alkyleneC₃₋₆heterocycloalkyl, OC₁alkyleneC₃₋₆heterocycloalkyl and OC₂alkyleneC₃₋₆heterocycloalkyl. Suitably the heterocycloalkyl is a heterocyclopropyl, heterocyclobutyl, heterocyclopentyl or heterocyclohexyl ring such as a heterocyclohexyl ring. Suitably, the heterocyclopentyl ring is tetrahydrofuranyl or pyrrolidinyl. Suitably, the heterocyclohexyl ring is tetrahydropyranyl or piperidinyl. Any nitrogen atom(s) (such as one nitrogen atom) in the C₃₋₆heterocycloalkyl ring may be substituted, for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄ haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃alkylOC₁₋₂alkyl such as C(O)CH₂OCH₃, and C₁₋₂alkylC(O)OC₁₋₄alkyl such as CH₂C(O)OCH₂CH₃. Suitably, any nitrogen atom in the C₃₋₄heterocycloalkyl ring is not substituted.

Suitably R_{5bb} is H, CH₃, ethyl, isopropyl or fluoro, in particular R_{5bb} is H, methyl or ethyl.

Suitably R_{4bb} is H, CH₃, ethyl, fluoro, OCH₃, propoxy or CH₂CH₂OCH₃ and R_{5bb} is H, CH₃, ethyl or fluoro, in particular R_{4bb} is H, CH₃, ethyl or OCH₃ and R_{5bb} is H, methyl or ethyl. For example, R_{4bb} and R_{5bb} are H, R_{4bb} and R_{5bb} are methyl, R_{4bb} and R_{5bb} are ethyl, R_{4bb} is CH₂CH₂OCH₃ and R_{5bb} is H or R_{4bb} and R_{5bb} are fluoro.

Suitably, when R_{4bb} is other than H, methyl, ethyl or fluoro, then R_{5bb} is H.

In one embodiment, A_{b} is A_{ab}. Suitably, A_{ab} is —NR_{6b}CH₂—. Alternatively, A_{ab} is —NR_{6b}—.

In another embodiment, A_{b} is A_{bb} i.e. —NR_{6b}C(═O)—.

In one embodiment R_{6b} is H. In a second embodiment R_{6b} is C₁₋₃alkyl, in particular methyl. In a third embodiment R_{6b} together with R_{11b} in the ortho-position to group A_{b} are a C₂alkylene chain forming a 5-membered ring. In a fourth embodiment, R_{4bb} is H and R_{5bb} and R_{6b} are a C₂₋₃alkylene chain forming a 5- or 6-membered ring in particular a 5-membered ring.

Suitably R_{6b} is H, methyl or R_{6b} together with R_{11b} when in the ortho-position to group A_{b} are a C₂alkylene chain forming a 5-membered ring. In particular R_{6b} is H or R_{6b} together with R_{11b} in the ortho-position to group A_{b} are a C₂alkylene chain forming a 5-membered ring, especially R_{6b} is H.

The term ‘R_{6b} together with R_{11b} in the ortho-position to group A_{b} are a C₂alkylene chain forming a 5-membered ring’ as used herein means that compounds with the following exemplary substructure are formed:

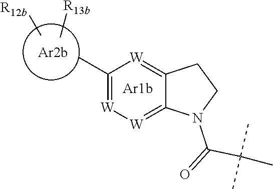

wherein W may be N or CR₁₀ₐ.

In one embodiment Ar1b is a 6-membered aryl, i.e. phenyl. In a second embodiment Ar1b is a 6-membered heteroaryl, in particular containing one nitrogen atom (pyridyl) or two nitrogen atoms (pyridazinyl, pyrimidinyl or pyrazinyl).

In particular Ar1b is phenyl, 2-pyridyl, 3-pyridyl or 2,6-pyrimidinyl, especially phenyl, 2-pyridyl or 3-pyridyl, such as phenyl or 2-pyridyl.

In one embodiment R_{10b} is H. In a second embodiment R_{10b} is halo, for example fluoro or chloro. In a third embodiment R_{10b} is C₁₋₃alkyl, such as CH₃, ethyl or isopropyl, in particular C₁₋₂alkyl, such as CH₃ or ethyl. In a fourth embodiment R_{10b} is OC₁₋₂alkyl, such as OCH₃ or ethoxy. In a fifth embodiment R_{10b} is C₁₋₂haloalkyl, such as CF₃. In a sixth embodiment R_{10b} is OC₁₋₂haloalkyl, such as OCF₃. In a seventh embodiment R_{10b} is CN.

Suitably R_{10b} is H, fluoro, chloro, CH₃, OCH₃, ethoxy, OCF₃ or CN, in particular H, fluoro, chloro, CH₃, OCH₃, ethoxy or OCF₃, especially or H, fluoro, chloro, CH₃, OCH₃ or OCF₃, such as H, fluoro or CH₃.

Additionally of interest are compounds wherein R_{10b} is ethyl, isopropyl and CF₃, in particular isopropyl and CF₃. Additionally of interest are compounds when R_{10b} is CN.

Consequently, suitably R_{10b} is H, fluoro, chloro, CH₃, ethyl, isopropyl, OCH₃, ethoxy, OCF₃, CF₃ or CN, in particular H, fluoro, chloro, CH₃, isopropyl, OCH₃, ethoxy, OCF₃ or CF₃, especially or H, fluoro, chloro, CH₃, isopropyl, OCH₃, OCF₃ or CF₃, such as H, fluoro or CH₃.

In one embodiment R_{11b} is H. In a second embodiment R_{11b} is F. In a third embodiment, R_{11b} is CH₃. In a fourth embodiment R_{6b} together with R_{11b} in the ortho-position to group A_{b} are a C₂alkylene chain forming a 5-membered ring. In a fifth embodiment R_{11b} is ethyl. In a sixth embodiment R_{11b} is Cl. In a seventh embodiment R_{11b} is OCH₃. In an eighth embodiment, R₁₁, is CF₃. In a ninth embodiment, R_{11b} is OCF₃. In a tenth embodiment, R_{11b} is CN. In an eleventh embodiment R_{6b} together with R_{11b} in the ortho-position to group A_{b} are a C₂alkylene chain forming a 5-membered ring.

Suitably, R_{10b} and R_{11b} are both CH₃. Suitably, R_{10b} and R_{11b} are both H. Suitably, R_{10b} and R_{11b} are both fluoro.

In one embodiment, R_{10b} is in the ortho position with respect to group A_{b}. In another embodiment, R_{10b} is in the meta position with respect to group A_{b}. Suitably R_{10b} is in the ortho position with respect to group A_{b}.

In one embodiment, R_{11b} is in the ortho position with respect to group A_{b}. In another embodiment, R_{11b} is in the meta position with respect to group A_{b}. Suitably R_{11b} is in the ortho position with respect to group A_{b}.

In one embodiment Ar2b is a 6-membered aryl, i.e. phenyl. In a second embodiment Ar2b is a 6-membered heteroaryl, in particular containing one nitrogen atom (pyridyl) or two nitrogen atoms (pyridazinyl, pyrimidinyl or pyrazinyl).

In particular Ar2b is phenyl, 2-pyridyl, 3-pyridyl, 4-pyridyl, 2,3-pyridazinyl, 3,4-pyridazinyl, 3,5-pyrimidinyl or 2,5-pyrazinyl, especially 3-pyridyl, 3,5-pyrimidinyl or 2,5-pyrazinyl, such as 3-pyridyl or 2,5-pyrazinyl.

In one embodiment R_{12b} is H. In a second embodiment R_{12b} is halo, for example fluoro or chloro. In a third embodiment R_{12b} is C₁₋₄alkyl, such as methyl, ethyl, propyl (n-propyl or isopropyl) or butyl (n-butyl, isobutyl, sec-butyl or tert-butyl). In a fourth embodiment R_{12b} is C₂₋₄alkynyl, such as C≡CH. In a fifth embodiment R_{12b} is C₀₋₂alkyleneC₃₋₅cycloalkyl, such as C₃₋₅cycloalkyl (e.g. cyclopropyl), C₁alkyleneC₃₋₅cycloalkyl or C₂alkyleneC₃₋₅cycloalkyl. In a sixth embodiment R_{12b} is OC₁₋₄alkyl, such as OCH₃, ethoxy, isopropoxy or n-propoxy. In a seventh embodiment R_{12b} is OC₀₋₂alkyleneC₃₋₅cycloalkyl, such as OC₃₋₅cycloalkyl (e.g. cyclopropoxy or cyclobutoxy), OC₁alkyleneC₃₋₅cycloalkyl or OC₂alkyleneC₃₋₅cycloalkyl. In an eighth embodiment R_{12b} is OCH₂CH₂N(CH₃)₂. In a ninth embodiment R_{12b} is C₁₋₄alkylOH, such as CH₂OH or C(CH₃)₂OH. In a tenth embodiment R_{12b} is CN. In an eleventh embodiment R_{12b} is C₁₋₃alkyleneOC₁₋₃alkyl. In a twelfth embodiment R_{12b} is C₁₋₄haloalkyl, such as CF₃. In a thirteenth embodiment R_{12b} is OC₁₋₄ haloalkyl, such as OCF₃, OCHF₂ or OCH₂CF₃. In a fourteenth embodiment R_{12b} is NR_{23b}R_{24b} such as N(CH₃)₂. In a fifteenth embodiment R_{12b} is S(O)₂C₁₋₄alkyl such as SO₂CH₃. In a sixteenth embodiment R_{12b} is C(O)N(CH₃)₂. In a seventeenth embodiment R_{12b} is NHC(O)C₁₋₃alkyl such as NHC(O)CH₃. In an eighteenth embodiment R_{12b} is a C₃₋₆heterocyloalkyl comprising one nitrogen located at the point of attachment to Ar2b, such as a C₅heterocycloalkyl, in particular pyrrolidinyl, or a C₆heterocycloalkyl such as morpholinyl. In a nineteenth embodiment R_{12b} is OH. In a twentieth embodiment R_{12b} is C(═O)C₁₋₂alkyl. In a twenty first embodiment R_{12b} is S(O)C₁₋₄alkyl. In a twenty second embodiment R_{12b} is SC₁₋₄alkyl. In a twenty third embodiment R_{12b} is SH. In a twenty fourth embodiment, R_{12b} together with a nitrogen atom to which it is attached forms an N-oxide (N⁺—O⁻).

R_{12b} is suitably H, fluoro, chloro, CH₃, cyclopropyl, C≡CH, OCH₃, ethoxy, n-propoxy, isopropoxy, cyclopropoxy, cyclobutoxy, CN, CF₃, OCF₃, OCHF₂, OCH₂CF₃, CH₂OH, N(CH₃)₂, NHC(O)CH₃, SO₂CH₃, C(O)N(CH₃)₂ or pyrrolidinyl, in particular H, fluoro, chloro, CH₃, cyclopropyl, C≡CH, OCH₃, ethoxy, n-propoxy, isopropoxy, cyclopropoxy, cyclobutoxy, CN, CF₃, OCF₃, OCHF₂, OCH₂CF₃, CH₂OH, C(O)N(CH₃)₂ or pyrrolidinyl, especially H, fluoro, chloro, CH₃, cyclopropyl, C≡CH, OCH₃, ethoxy, n-propoxy, isopropoxy, cyclopropoxy, CN, CF₃, OCHF₂, OCH₂CF₃ or pyrrolidinyl, such as H, fluoro, chloro, CH₃, C≡CH, OCH₃, ethoxy, n-propoxy, isopropoxy, cyclopropoxy, CN, CF₃, OCHF₂ or OCH₂CF₃.

Additionally of interest are ethyl, 2-methoxyisopropyl and OH, especially ethyl.

In one embodiment, R_{23b} is H. In another embodiment, R_{23b} is C₁₋₂alkyl such as methyl.

In one embodiment, R_{24b} is H. In another embodiment R_{24b} is C₁₋₂alkyl such as methyl.

Suitably, R_{23b} is H and R_{24b} is ethyl. Suitably, R_{23b} is CH₃ and R_{24b} is CH₃.

Consequently, suitably R_{12b} is H, fluoro, chloro, CH₃, ethyl, cyclopropyl, C≡CH, OCH₃, ethoxy, n-propoxy, isopropoxy, cyclopropoxy, cyclobutoxy, CN, CF₃, OCF₃, OCHF₂, OCH₂CF₃, OH, CH₂OH, N(CH₃)₂, NHC(O)CH₃, SO₂CH₃, C(O)N(CH₃)₂ or pyrrolidinyl, in particular H, fluoro, chloro, CH₃, ethyl, cyclopropyl, C≡CH, OCH₃, ethoxy, n-propoxy, isopropoxy, cyclopropoxy, cyclobutoxy, CN, CF₃, OCF₃, OCHF₂, OCH₂CF₃, CH₂OH, C(O)N(CH₃)₂ or pyrrolidinyl, especially H, fluoro, chloro, CH₃, ethyl, cyclopropyl, C≡CH, OCH₃, ethoxy, n-propoxy, isopropoxy, cyclopropoxy, CN, CF₃, OCHF₂, OCH₂CF₃ or pyrrolidinyl, such as H, fluoro, chloro, CH₃, C≡CH, OCH₃, ethoxy, n-propoxy, isopropoxy, cyclopropoxy, CN, CF₃, OCHF₂ or OCH₂CF₃.

Suitably R_{12b} is suitably in the meta position of Ar2b. Alternatively, R_{12b} is in the ortho position of Ar2b.

In one embodiment R_{13b} is methyl. In a second embodiment R_{13b} is H. In a third embodiment R_{13b} is methoxy. In a fourth embodiment R_{13b} is halo such as fluoro.

In one embodiment, R_{13b} is in the ortho position with respect to Ar1b. In another embodiment, R_{13b} is in the para position with respect to Ar1b.

The present invention provides compound T466.

The present invention provides the following compound:

### N-(4-(1-((2-fluoro-4-(pyridin-3-yl)phenyl)amino)-2-methylpropan-2-yl)thiazol-2-yl)cyclopropanesulfonamide

### Compounds of Formula (I-c)

The invention provides a compound of formula (I-c):

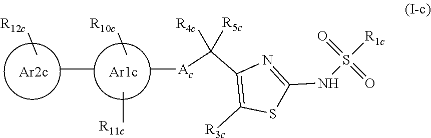

wherein
A_{c} is A_{ac} or A_{bc};
   A_{ac} is —CH₂NR_{6c}—;
   A_{bc} is —C(═O)NR_{6c}—;
R_{1c} is R_{1ac} or R_{1bc};
wherein:
   R_{1ac} is NR_{32c}R_{33c};
R_{1bc} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R_{3c} is H, CH₃, halo, OC₁₋₂alkyl or CF₃;
R_{4c} and R_{5c} are either R_{4ac} and R_{5ac} or R_{4bc} and R_{5bc};
wherein:
   R_{4ac} and R_{5ac} together with the carbon atom to which they are attached form a C₃₋₆cycloalkyl which is:
      substituted by one or two substituents, each substituent being independently selected from the group consisting of C₁₋₃alkyl, oxo, OH, C₁₋₃ alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21c}R_{22c}; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl; or
   R_{4ac} and R_{5ac} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heteroycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl; or
   R_{4ac} and R_{5ac} together with the carbon atom to which they are attached form a C₃₋₆heterocycloalkyl comprising one nitrogen atom, wherein said nitrogen atom is substituted by —S(O)₂R_{29c}; or
   R_{4ba} and R_{5bc} are each independently H, C₁₋₆alkyl, C₀₋₂alkyleneC₃₋₆cycloalkyl, C₀₋₂alkyleneC₃₋₆heterocycloalkyl, C₁₋₃alkyleneOC₁₋₃alkyl, C₁₋₆alkylOH or C₁₋₆haloalkyl, or R_{4bc} and R_{5bc} together with the carbon atom to which they are attached form a C₃₋₆cycloalkyl or C₃₋₆heterocycloalkyl ring;
R_{6c} is H or C₁₋₃alkyl;
Ar1c is a 6-membered aryl or heteroaryl;
Ar2c is a 6-membered aryl or heteroaryl and is attached to Ar1c in the para position relative to group A_{c};
R_{10c} is H, halo, C₁₋₃alkyl, OC₁₋₂alkyl, C₁₋₂haloalkyl, OC₁₋₂haloalkyl or CN;
R_{11c} is H, F, Cl, CH₃, ethyl, OCH₃, CF₃, OCF₃ or CN;
R_{12c} is attached to Ar2c in the meta or ortho position relative to Ar1c and R_{12c} is H, halo, C₁₋₄alkyl, C₂₋₄alkynyl, C(═O)C₁₋₂alkyl, C₀₋₂alkyleneC₃₋₅cycloalkyl, OC₁₋₄alkyl, C₁₋₃ alkyleneOC₁₋₃alkyl, C₁₋₄haloalkyl, OC₁₋₄haloalkyl, CN, OC₀₋₂alkyleneC₃₋₅cycloalkyl, OCH₂CH₂N(CH₃)₂, OH, C₁₋₄alkylOH, NR_{23c}R_{24c}, SO₂CH₃, C(O)N(CH₃)₂, NHC(O)C₁₋₃alkyl, or a C₃₋₆heterocycloalkyl comprising one nitrogen located at the point of attachment to Ar2c, or R_{12c} together with a nitrogen atom to which it is attached forms an N-oxide (N⁺—O⁻);
R_{21c} is H, C₁₋₅alkyl, C(O)C₁₋₅alkyl, C(O)OC₁₋₅alkyl, C₁₋₃alkylOC₁₋₂alkyl, C₁₋₄haloalkyl, or C₄₋₆heterocycloalkyl;
R_{22c} is H or CH₃;
R_{23c} is H or C₁₋₂alkyl;
R_{24c} is H or C₁₋₂alkyl;
R_{29c} is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl; and
R_{32c} is C₁₋₃alkyl and R_{33c} is C₁₋₃alkyl; or
R_{32c} and R_{33c} together with the nitrogen atom to which they are attached form a C₃₋₆heterocycloalkyl;

or a salt and/or solvate thereof and/or derivative thereof.

Suitably, R_{1c} is R_{1ac}; and/or R_{4c} and R_{5c} are R_{4ac} and R_{5ac}; and/or A_{c} is A_{ac}.

The invention also provides a compound of formula (I-c):

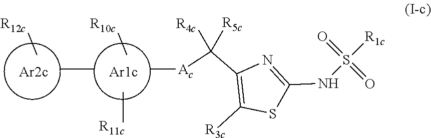

wherein
A_{c} is A_{ac} or A_{bc};
   A_{ac} is —CH₂NR_{6c}—;
   A_{bc} is —C(═O)NR_{6c}—;
R_{1c} is R_{1ac} or R_{1bc};
wherein:
   R_{1ac} is NR_{32c}R_{33c};
R_{1bc} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R_{3c} is H, CH₃, halo, OC₁₋₂alkyl or CF₃;
R_{4c} and R_{5c} are either R_{4ac} and R_{5ac} or R_{4bc} and R_{5bc};
wherein:
   R_{4ac} and R_{5ac} together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21c}R_{22c}; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ac} and R_{5ac} together with the carbon atom to which they are attached may be substituted by one or two substituents, each substituent being independently selected from the group consisting of C₁₋₃ alkyl or OC₁₋₃alkyl; or
   R_{4ac} and R_{5ac} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heteroycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl; or
   R_{4ac} and R_{5ac} together with the carbon atom to which they are attached form a C₃₋₆heterocycloalkyl comprising one nitrogen atom, wherein said nitrogen atom is substituted by —S(O)₂R_{29c}; or
   R_{4bc} and R_{5bc} are each independently H, C₁₋₆alkyl, C₀₋₂alkyleneC₃₋₆cycloalkyl, C₀₋₂alkyleneC₃₋₆heterocycloalkyl, C₁₋₃alkyleneOC₁₋₃alkyl, C₁₋₆alkylOH or C₁₋₆haloalkyl,
   or R_{4bc} and R_{5bc} together with the carbon atom to which they are attached form a C₃₋₆cycloalkyl or C₃₋₆heterocycloalkyl ring;
R_{6c} is H or C₁₋₃alkyl;
Ar1c is a 6-membered aryl or heteroaryl;
Ar2c is a 6-membered aryl or heteroaryl and is attached to Ar1c in the para position relative to group A_{c};
R_{10c} is H, halo, C₁₋₃alkyl, OC₁₋₂alkyl, C₁₋₂haloalkyl, OC₁₋₂haloalkyl or CN;
R_{11c} is H, F, Cl, CH₃, ethyl, OCH₃, CF₃, OCF₃ or CN;
R_{12c} is attached to Ar2c in the meta or ortho position relative to Ar1c and R_{12c} is H, halo, C₁₋₄alkyl, C₂₋₄alkynyl, C(═O)C₁₋₂alkyl, C₀₋₂alkyleneC₃₋₅cycloalkyl, OC₁₋₄alkyl, C₁₋₃ alkyleneOC₁₋₃alkyl, C₁₋₄haloalkyl, OC₁₋₄haloalkyl, CN, OC₀₋₂alkyleneC₃₋₅cycloalkyl, OCH₂CH₂N(CH₃)₂, OH, C₁₋₄alkylOH, NR_{23c}R_{24c}, SO₂CH₃, C(O)N(CH₃)₂, NHC(O)C₁₋₃alkyl, or a C₃₋₆heterocycloalkyl comprising one nitrogen located at the point of attachment to Ar2c, or R_{12c} together with a nitrogen atom to which it is attached forms an N-oxide (N⁺—O⁻);
R_{21c} is H, C₁₋₅alkyl, C(O)C₁₋₅alkyl, C(O)OC₁₋₅alkyl;
R_{22c} is H or CH₃;
R_{23c} is H or C₁₋₂alkyl;
R_{24c} is H or C₁₋₂alkyl;
R_{29c} is C₁₋₃alkyl, C₀₋₂alkyleneC₃₋₅cycloalkyl which cycloalkyl is optionally substituted by CH₃, or CF₃; and
R_{32c} is C₁₋₃alkyl and R_{33c} is C₁₋₃alkyl; or
R_{32c} and R₃₃ together with the nitrogen atom to which they are attached form a C₃₋₅heterocycloalkyl;

or a salt and/or solvate thereof and/or derivative thereof.

Suitably, R_{1c} is R_{1ac}; and/or R_{4c} and R_{5c} are R_{4ac} and R_{5ac}; and/or A_{c} is A_{ac}.

When R_{4bc} and/or R_{5bc} is C₀alkyleneC₃₋₆heterocycloalkyl, any heteroatom in the heterocycloalkyl may not be directly connected to the carbon to which R_{4bc} and R_{5bc} are connected.

In one embodiment of the invention, R_{1c} is R_{1ac}, i.e. is NR_{32c}R_{33c}. In an embodiment, R_{32c} is C₁₋₃ alkyl, such as methyl or ethyl, e.g. methyl. In an embodiment, R_{33c} is C₁₋₃alkyl, such as methyl or ethyl, e.g. methyl. Suitably, R_{32c} and R_{33c} are both methyl. Suitably, R_{32c} and R_{33c} are both ethyl. Suitably, R_{32c} is methyl and R_{33c} is ethyl.

In another embodiment, R_{32c} and R_{33c} together with the nitrogen atom to which they are attached form a C₃₋₅heterocycloalkyl. Suitably, the C₃₋₅heterocycloalkyl is aziridinyl, azetidinyl or pyrrolidinyl.

Suitably, R_{1c} is R_{1bc}.

In one embodiment of the invention R_{1bc} is C₁₋₅alkyl such as C₁₋₄alkyl. When R_{1bc} is C₁₋₅alkyl, R_{1bc} is methyl, ethyl, propyl (n-propyl or isopropyl), butyl (n-butyl, isobutyl, sec-butyl or tert-butyl) or pentyl (e.g. n-pentyl, sec-pentyl, 3-pentyl, sec-isopentyl or active pentyl). When R_{1bc} is C₁₋₄alkyl, R_{1bc} is methyl, ethyl, propyl (n-propyl or isopropyl) or butyl (n-butyl, isobutyl, sec-butyl or tert-butyl).

In a second embodiment of the invention R_{1bc} is C₁₋₃alkyleneOC₁₋₂alkyl such as C₁₋₂alkyleneOC₁₋₂alkyl. R_{1bc} may be C₁alkyleneOC₁alkyl. R_{1bc} may be C₁alkyleneOC₂alkyl. R_{1bc} may be C₂alkyleneOC₁alkyl. R_{1bc} may be C₂alkyleneOC₂alkyl. R_{1bc} may be C₃alkyleneOC₁alkyl. R_{1bc} may be C₃alkyleneOC₂alkyl.

In a third embodiment of the invention R_{1bc} is C₀₋₂alkyleneC₃₋₅cycloalkyl which cycloalkyl is optionally substituted by CH₃ such as C₀₋₁alkyleneC₃₋₅cycloalkyl which cycloalkyl is optionally substituted by CH₃. In some embodiments, R_{1bc} is C₀₋₂alkyleneC₃₋₅cycloalkyl such as C₀₋₁alkyleneC₃₋₄cycloalkyl. In other embodiments, R_{1bc} is C₀₋₂alkyleneC₃₋₅cycloalkyl which cycloalkyl is substituted by CH₃ such as C₀₋₁alkyleneC₃₋₄cycloalkyl which cycloalkyl is substituted by CH₃. R_{1bc} may be C₃₋₅cycloalkyl, which cycloalkyl is optionally substituted by CH₃ such as C₃₋₄cycloalkyl, which cycloalkyl is optionally substituted by CH₃. R_{1bc} may be C₁alkyleneC₃₋₅cycloalkyl, which cycloalkyl is optionally substituted by CH₃. R_{1bc} may be C₁alkyleneC₃₋₄cycloalkyl, which cycloalkyl is optionally substituted by CH₃. R_{1bc} may be C₂alkyleneC₃₋₅cycloalkyl, which cycloalkyl is optionally substituted by CH₃. R_{1bc} may be C₂alkyleneC₃₋₄cycloalkyl, which cycloalkyl is optionally substituted by CH₃. R_{1bc} may be C₀₋₂alkyleneC₃cycloalkyl, which cycloalkyl is optionally substituted by CH₃. R_{1bc} may be C₀₋₁alkyleneC₃cycloalkyl, which cycloalkyl is optionally substituted by CH₃. R_{1bc} may be C₀₋₂alkyleneC₄cycloalkyl, which cycloalkyl is optionally substituted by CH₃. R_{1bc} may be C₀₋₁alkyleneC₄cycloalkyl, which cycloalkyl is optionally substituted by CH₃. R_{1bc} may be C₀₋₂alkyleneC₅cycloalkyl, which cycloalkyl is optionally substituted by CH₃. R_{1bc} may be C₀₋₁alkyleneC₅cycloalkyl, which cycloalkyl is optionally substituted by CH₃. Suitably, where C₀₋₂alkyleneC₃₋₅cycloalkyl such as C₀₋₁alkyleneC₃₋₄cycloalkyl is optionally substituted by CH₃, the CH₃ is at the point of attachment of the C₃₋₅cycloalkyl to the C₀₋₂alkylene such as at the point of attachment of the C₃₋₄cycloalkyl to the C₀₋₁alkylene.

Suitably R_{1bc} is cyclopropyl.

In a fourth embodiment of the invention, R_{1bc} is CF₃.

In one embodiment R_{3c} is H. In a second embodiment R_{3c} is CH₃. In a third embodiment, R_{3c} is halo. In an example, R_{3c} is F. In a second example, R_{3c} is Cl. In a fourth embodiment, R_{3c} is OC₁₋₂alkyl. Suitably R_{3c} is OCH₃. Suitably, R_{3c} is OCH₂CH₃. In a fifth embodiment, R_{3c} is CF₃.

Suitably, R_{3c} is H.

In one embodiment, R_{4c} and R_{5c} are R_{4ac} and R_{5ac}.

Suitably, R_{4ac} and R_{5ac} together with the carbon atom to which they are attached form a C₃₋₆cycloalkyl which is:
substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21c}R_{22c}.

In one embodiment, the C₃₋₆cycloalkyl is cyclopropyl. In another embodiment, the C₃₋₆cycloalkyl is cyclobutyl. In another embodiment, the C₃₋₆cycloalkyl is cyclopentyl. In another embodiment, the C₃₋₆cycloalkyl is cyclohexyl.

In one embodiment the C₃₋₆cycloalkyl is substituted by one substituent. In a second embodiment the C₃₋₆cycloalkyl is substituted by two substituents.

In one embodiment, the substituent is C₁₋₃alkyl. Suitably, the substituent is methyl. Suitably, the substituent is ethyl. Suitably, the substituent is n-propyl. Suitably, the substituent is iso-propyl.

In a second embodiment, the substituent is C₁₋₃alkylOH. Suitably, the substituent is CH₂OH. Suitably, the substituent is CH₂CH₂OH. Suitably, the substituent is CH₂CH₂CH₂OH.

In a third embodiment, the substituent is C₁₋₃haloalkyl. Suitably the C₁₋₃alkyl group is substituted by one two or three, such as one, halogen atom. Suitably, the halogen atom is fluoro or chloro such as fluoro. Suitably, the substituent is C₁haloalkyl such as CF₃. Suitably, the substituent is C₂haloalkyl such as CH₂CF₃.

In a fourth embodiment, the substituent is C₀₋₂alkyleneC₃₋₆cycloalkyl, in particular C₀₋₂alkyleneC₃₋₅cycloalkyl, such as C₃₋₅cycloalkyl, C₁alkyleneC₃₋₅cycloalkyl or C₂alkyleneC₃₋₅cycloalkyl.

In a fifth embodiment, the substituent is C₀₋₂alkyleneC₃₋₆heterocycloalkyl such as C₀₋₂alkyleneC₃heterocycloalkyl, C₀₋₂alkyleneC₄heterocycloalkyl, C₀₋₂alkyleneC₅heterocycloalkyl, C₀₋₂alkyleneC₆heterocycloalkyl, C₀alkyleneC₃₋₆heterocycloalkyl, C₁alkyleneC₃₋₆heterocycloalkyl and C₂alkyleneC₃₋₆heterocycloalkyl. Suitably the heterocycloalkyl is a heterocyclopropyl, heterocyclobutyl, heterocyclopentyl or heterocyclohexyl ring such as a heterocyclohexyl ring. Suitably, the heterocyclopentyl ring is tetrahydrofuranyl or pyrrolidinyl. Suitably, the heterocyclohexyl ring is tetrahydropyranyl or piperidinyl. Any nitrogen atom(s) in the C₃₋₆heterocycloalkyl ring may be substituted (such as one nitrogen atom is substituted), for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃ alkylOC₁₋₂alkyl such as C(O)CH₂OCH₃, and C₁₋₂alkylC(O)OC₁₋₄alkyl such as CH₂C(O)OCH₂CH₃. Suitably, any nitrogen atom in the C₃₋₆heterocycloalkyl ring is not substituted.

In a sixth embodiment, the substituent is C₁₋₃alkyleneOC₁₋₃alkyl, in particular C₁₋₂alkyleneOC₁₋₂alkyl such as C₁alkyleneOC₁alkyl, C₂alkyleneOC₁alkyl, C₁alkyleneOC₂alkyl or C₂alkyleneOC₂alkyl.

In a seventh embodiment, the substituent is halo, in particular fluoro or chloro such as chloro.

In an eighth embodiment, the substituent is OC₁₋₃haloalkyl. Suitably the OC₁₋₃alkyl group is substituted by one two or three, such as one, halogen atom. Suitably, the halogen atom is fluoro or chloro such as fluoro. Suitably, the substituent is OC₁haloalkyl such as OCF₃. Suitably, the substituent is OC₂haloalkyl such as OCH₂CF₃.

In a ninth embodiment, the substituent is OC₀₋₂alkyleneC₃₋₆cycloalkyl, such as OC₃₋₆cycloalkyl, OC₁alkyleneC₃₋₆cycloalkyl or OC₂alkyleneC₃₋₆cycloalkyl.

In a tenth embodiment, the substituent is OC₀₋₂alkyleneC₃₋₆heterocycloalkyl such as OC₀₋₂alkyleneC₃heterocycloalkyl, OC₀₋₂alkyleneC₄heterocycloalkyl, OC₀₋₂alkyleneC₅heterocycloalkyl, OC₀₋₂alkyleneC₆heterocycloalkyl, OC₀alkyleneC₃₋₆heterocycloalkyl, OC₁alkyleneC₃₋₆heterocycloalkyl and OC₂alkyleneC₃₋₆heterocycloalkyl. Suitably the heterocycloalkyl is a heterocyclopropyl, heterocyclobutyl, heterocyclopentyl or heterocyclohexyl ring such as a heterocyclohexyl ring. Suitably, the heterocyclopentyl ring is tetrahydrofuranyl or pyrrolidinyl. Suitably, the heterocyclohexyl ring is tetrahydropyranyl or piperidinyl. Any nitrogen atom(s) (such as one nitrogen atom) in the C₃₋₆heterocycloalkyl ring may be substituted, for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄ haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃alkylOC₁₋₂alkyl such as C(O)CH₂OCH₃, and C₁₋₂alkylC(O)OC₁₋₄alkyl such as CH₂C(O)OCH₂CH₃. Suitably, any nitrogen atom in the C₃₋₆heterocycloalkyl ring is not substituted.

In an eleventh embodiment, the substituent is OC₁₋₃alkyl, such as OCH₃ or OCH₂CH₃.

In a twelfth embodiment, the substituent is NR_{21c}R_{22c}. In one embodiment R_{21c} is H. In a second embodiment R_{21c} is C₁₋₅alkyl, such as methyl, ethyl or propyl, especially methyl. In a third embodiment R_{21c} is C(O)C₁₋₅alkyl, such as C(O)CH₃. In a fourth embodiment R_{21c} is C(O)OC₁₋₅alkyl, such as C(O)OCH₃ or C(O)Otert-butyl. In a fifth embodiment R_{21c} is C₁₋₃alkylOC₁₋₂alkyl such as C₁alkylOC₁alkyl, C₂alkylOC₁alkyl or C₃alkylOC₁alkyl e.g. C₂alkylOC₁alkyl. In a sixth embodiment, R_{21c} is C₁₋₄haloalkyl, such as CF₃, CH₂CF₃ or CH₂CHF₂ e.g. CH₂CHF₂. In a seventh embodiment R_{21c} is C₄₋₆heterocycloalkyl, such as oxetanyl, tetrahydrofuranyl or tetrahydropyranyl e.g. oxetanyl, in particular 3-oxetanyl.

When the substituent is NR_{21c}R_{22c}, in one embodiment R_{22c} is H. In a second embodiment R_{22c} is methyl.

Suitably, R_{21c} is C(O)OCH₃ and R_{22c} is H. Suitably, R_{21c} is C(O)CH₃ and R_{22c} is H. Suitably, R_{21c} and R_{22c} are both CH₃. Suitably, R_{21c} and R_{22c} are both H.

In a thirteenth embodiment, the substituent is oxo.

In a fourteenth embodiment, the substituent is OH.

Suitably, the one or two substituents, in particular one substituent, are independently selected from the group consisting of C₁₋₃alkyl, oxo, OH, C₁₋₃alkylOH, C₁₋₃haloalkyl, halo, OC₁₋₃haloalkyl, OC₁₋₃alkyl and NR_{21c}R_{22c}.

More suitably, the substituent is independently selected from the group consisting of oxo, OH, halo, OC₁₋₃alkyl and NR_{21c}R_{22c}.

Most suitably, the substituent is independently selected from the group consisting of oxo, OH, fluoro, NR_{21c}R_{22c}.

Alternatively, R_{4ac} and R_{5ac} together with the carbon atom to which they are attached form a C₃₋₆cycloalkyl and one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl.

In one embodiment the C₃₋₆cycloalkyl formed by R_{4ac} and R_{5ac} together with the carbon atom to which they are attached is unsubstituted. In a second embodiment the C₃₋₆cycloalkyl formed by R_{4ac} and R_{5ac} together with the carbon atom to which they are attached is substituted by one or two substituents, in particular one substituent. Suitably, each substituent is independently selected from the group consisting of C₁₋₂alkyl or OCH₃.

Suitably one of the carbons of the C₃₋₆cycloalkyl which is formed by R_{4ac} and R_{5ac} is a spiro centre such that a spirocyclic ring system is formed, wherein the C₃₋₆cycloalkyl which is formed by R_{4ac} and R_{5ac} is a C₄₋₆cycloalkyl. Suitably the C₃₋₆heterocycloalkyl is an oxygen containing C₃₋₆heterocycloalkyl. Suitably, the C₃₋₆heterocycloalkyl is an oxygen comprising, such as containing, C₃₋₆heterocycloalkyl ring, such as a C₅cycloalkyl ring.

In an embodiment, R_{4ac} and R_{5ac} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heteroycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl. Suitably, each substituent is independently selected from the group consisting of C₁₋₂alkyl or OCH₃.

Suitably one of the carbons of the C₃₋₆heterocycloalkyl is a spiro centre such that a spirocyclic ring system is formed containing further ring C, wherein C is a C₄₋₆heterocycloalkyl. Suitably the C₄₋₆heterocycloalkyl is an oxygen containing C₄₋₆heterocycloalkyl such as tetrahydropyranyl or 1,3-dioxolanyl.

As stated above, when a heterocycloalkyl is formed from R_{4c} and R_{5c} together with the carbon atom to which they are attached, suitably any heteroatom is not directly connected to the carbon to which R_{4c} and R_{5c} are attached.

In an embodiment, R_{4ac} and R_{5ac} together with the carbon atom to which they are attached form a C₃₋₆heterocycloalkyl comprising one nitrogen atom, wherein said nitrogen atom is substituted by —S(O)₂R_{29c}.

Suitably, the C₃₋₆heterocycloalkyl is selected from the group consisting of aziridinyl, azetidinyl, pyrrolidinyl and piperidinyl such as piperidinyl.

Suitably, when the C₃₋₆heterocycloalkyl is piperidinyl, the nitrogen atom is in the 4-position relative to the quaternary carbon:

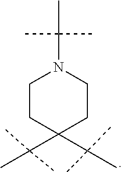

The C₃₋₆ heterocycloalkyl may be other groups as defined elsewhere herein.

In an embodiment, R_{29c} is C₁₋₃alkyl, C₀₋₂alkyleneC₃₋₅cycloalkyl which cycloalkyl is optionally substituted by CH₃, or CF₃. In one embodiment, R_{29c} is C₁₋₃alkyl such as methyl. In another embodiment, R_{29c} is C₀₋₂alkyleneC₃₋₅cycloalkyl which cycloalkyl is optionally substituted by CH₃. In some embodiments, R_{29c} is C₀₋₂alkyleneC₃₋₅cycloalkyl. In other embodiments, R_{29c} is C₀₋₂alkyleneC₃₋₅cycloalkyl which cycloalkyl is substituted by CH₃. R_{29c} may be C₃₋₅cycloalkyl, which cycloalkyl is optionally substituted by CH₃. R_{29c} may be C₁alkyleneC₃₋₅cycloalkyl, which cycloalkyl is optionally substituted by CH₃. R_{29c} may be C₂alkyleneC₃₋₅cycloalkyl, which cycloalkyl is optionally substituted by CH₃. R_{29c} may be C₀₋₂alkyleneC₃cycloalkyl, which cycloalkyl is optionally substituted by CH₃. R_{29c} may be C₀₋₂alkyleneC₄cycloalkyl, which cycloalkyl is optionally substituted by CH₃. R_{29c} may be C₀₋₂alkyleneC₅cycloalkyl, which cycloalkyl is optionally substituted by CH₃. Suitably, where C₀₋₂alkyleneC₃₋₅cycloalkyl is optionally substituted by CH₃, the CH₃ is at the point of attachment of the C₃₋₅cycloalkyl to the C₀₋₂alkylene. In another embodiment, R_{29c} is CF₃. In another embodiment, R_{29c} is N(C₁₋₃alkyl)₂ such as N(CH₃)₂. In another embodiment, R_{29c} is a 5 or 6 membered heteroaryl wherein the 5 or 6 membered heteroaryl is optionally substituted by methyl. In one embodiment, the 5 or 6 membered heteroaryl is substituted by methyl. In one embodiment, the 5 or 6 membered heteroaryl is not substituted by methyl. In one embodiment, R_{29c} is a 5-membered heteroaryl such as pyrrolyl, furanyl, thiophenyl, imidazolyl, pyrazolyl, oxazolyl, isoxazolyl, thiazolyl, isothiazolyl, such as pyrazolyl. Suitably the pyrazolyl is substituted by methyl. In another embodiment, R_{29c} is a 6-membered heteroaryl such as pyridinyl, pyridazinyl, pyrimidinyl or pyrazinyl.

In another embodiment, R_{4c} and R_{5c} are R_{4bc} and R_{5bc}.

In one embodiment, R_{4bc} is H. In a second embodiment R_{4bc} is C₁₋₆alkyl such as C₁₋₄alkyl, i.e. methyl, ethyl, propyl (n-propyl or isopropyl) or butyl (n-butyl, isobutyl, sec-butyl or tert-butyl). R_{4bc} may also be pentyl (e.g. n-pentyl, sec-pentyl, 3-pentyl, sec-isopentyl or active pentyl) or hexyl (e.g. n-hexyl, 2-methylpentyl, 3-methylpentyl, 2,2-dimethylbutyl and 2,3-dimethylbutyl). In a third embodiment, R_{4bc} is C₀₋₂alkyleneC₃₋₆cycloalkyl such as C₀₋₂alkyleneC₃₋₅cycloalkyl, such as C₀₋₂alkyleneC₃cycloalkyl, C₀₋₂alkyleneC₄cycloalkyl, C₀₋₂alkyleneC₅cycloalkyl, C₀alkyleneC₃₋₅ cycloalkyl, C₁alkyleneC₃₋₅cycloalkyl and C₂alkyleneC₃₋₅cycloalkyl. R_{4bc} may also be C₀₋₂alkyleneC₆cycloalkyl, C₀alkyleneC₃₋₆cycloalkyl, C₁alkyleneC₃₋₆cycloalkyl and C₂alkyleneC₃₋₆cycloalkyl. In a fourth embodiment R_{4bc} is C₁₋₃alkyleneOC₁₋₃alkyl, in particular C₁₋₂alkyleneOC₁₋₂ alkyl such as C₁alkyleneOC₁alkyl, C₂alkyleneOC₁alkyl, C₁alkyleneOC₂alkyl or C₂alkyleneOC₂alkyl. In a fifth embodiment R_{4bc} is C₁₋₆alkylOH such as C₁₋₄alkylOH such as C₁alkylOH, C₂alkylOH, C₃alkylOH or C₄alkylOH wherein C₁₋₄alkyl is methyl, ethyl, propyl (n-propyl or isopropyl) and butyl (n-butyl, isobutyl, sec-butyl or tert-butyl). R_{4bc} may also be C₅alkylOH or C₆alkylOH. In a sixth embodiment, R_{4bc} is C₁₋₆haloalkyl such as C₁₋₄haloalkyl such as C₁haloalkyl (e.g. CF₃), C₂haloalkyl (e.g. CH₂CF₃), C₃haloalkyl (e.g. CH₂CH₂CF₃) or C₄haloalkyl (e.g. CH₂CH₂CH₂CF₃). R_{4bc} may also be C₅haloalkyl (e.g. CH₂CH₂CH₂CH₂CF₃) or C₆haloalkyl (e.g. CH₂CH₂CH₂CH₂CH₂CF₃). In a seventh embodiment, R_{4bc} is C₀₋₂alkyleneC₃₋₆heterocycloalkyl such as C₀₋₂alkyleneC₃heterocycloalkyl, C₀₋₂alkyleneC₄heterocycloalkyl, C₀₋₂alkyleneC₅heterocycloalkyl, C₀₋₂alkyleneC₆heterocycloalkyl, C₀alkyleneC₃₋₆heterocycloalkyl, C₁alkyleneC₃₋₆heterocycloalkyl and C₂alkyleneC₃₋₆heterocycloalkyl. Suitably the heterocycloalkyl of a C₀₋₂alkyleneC₃₋₆heterocycloalkyl group is a heterocyclopropyl, heterocyclobutyl, heterocyclopentyl or heterocyclohexyl ring such as a heterocyclohexyl ring. Suitably, the heterocyclopentyl ring is tetrahydrofuranyl or pyrrolidinyl. Suitably, the heterocyclohexyl ring is tetrahydropyranyl or piperidinyl. Any nitrogen atom such as one nitrogen atom in the C₃₋₆heterocycloalkyl ring may be substituted, for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄ haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃alkylOC₁₋₂alkyl such as C(O)CH₂OCH₃, and C₁₋₂alkylC(O)OC₁₋₄alkyl such as CH₂C(O)OCH₂CH₃. Suitably, any nitrogen atom in the C₃₋₆heterocycloalkyl ring is not substituted. In an eighth embodiment, R_{4bc} and R_{5bc} together with the carbon atom to which they are attached form a C₃₋₆cycloalkyl or C₃₋₆heterocycloalkyl ring. Suitably R_{4bc} and R_{5bc} together with the carbon atom to which they are attached form a C₃₋₆cycloalkyl ring, such as a cyclopropyl, cyclobutyl, cyclopentyl or cyclohexyl ring. Suitably R_{4bc} and R_{5bc} together with the carbon atom to which they are attached form a C₃₋₆heterocycloalkyl ring, such as a heterocyclopropyl, heterocyclobutyl, heterocyclopentyl or heterocyclohexyl ring. Suitably, the heterocyclopentyl ring is tetrahydrofuranyl or pyrrolidinyl. Suitably, the heterocyclohexyl ring is tetrahydropyranyl or piperidinyl. Any nitrogen atom such as one nitrogen atom in the C₃₋₆heterocycloalkyl ring may be substituted, for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃alkylOC₁₋₂alkyl such as C(O)CH₂OCH₃, and C₁₋₂alkylC(O)OC₁₋₄alkyl such as CH₂C(O)OCH₂CH₃. Suitably, any nitrogen atom in the C₃₋₆heterocycloalkyl ring is not substituted.

Suitably R_{4bc} is H, CH₃ or ethyl, in particular CH₃ or ethyl. Suitably, R_{4bc} and R_{5bc} together with the carbon atom to which they are attached form a C₃₋₆cycloalkyl ring, such as a cyclopropyl ring.

In one embodiment, R_{5bc} is H. In a second embodiment R_{5bc} is C₁₋₆alkyl such as C₁₋₄alkyl, i.e. methyl, ethyl, propyl (n-propyl or isopropyl) or butyl (n-butyl, isobutyl, sec-butyl or tert-butyl). R_{5bc} may also be pentyl (e.g. n-pentyl, sec-pentyl, 3-pentyl, sec-isopentyl and active pentyl) or hexyl (e.g. n-hexyl, 2-methylpentyl, 3-methylpentyl, 2,2-dimethylbutyl and 2,3-dimethylbutyl). In a third embodiment, R_{5bc} is C₀₋₂alkyleneC₃₋₆cycloalkyl such as C₀₋₂alkyleneC₃₋₅cycloalkyl, such as C₀₋₂alkyleneC₃cycloalkyl, C₀₋₂alkyleneC₄cycloalkyl, C₀₋₂alkyleneC₅cycloalkyl, C₀alkyleneC₃₋₅ cycloalkyl, C₁alkyleneC₃₋₅cycloalkyl and C₂alkyleneC₃₋₅cycloalkyl. R_{5bc} may also be C₀₋₂alkyleneC₆cycloalkyl, C₀alkyleneC₃₋₆cycloalkyl, C₁alkyleneC₃₋₆cycloalkyl and C₂alkyleneC₃₋₆cycloalkyl. In a fourth embodiment R_{5bc} is C₁₋₃alkyleneOC₁₋₃alkyl, in particular C₁₋₂alkyleneOC₁₋₂ alkyl such as C₁alkyleneOC₁alkyl, C₂alkyleneOC₁alkyl, C₁alkyleneOC₂alkyl or C₂alkyleneOC₂alkyl. In a fifth embodiment R_{5bc} is C₁₋₆alkylOH such as C₁₋₄alkylOH such as C₁alkylOH, C₂alkylOH, C₃alkylOH or C₄alkylOH wherein C₁₋₄alkyl is methyl, ethyl, propyl (n-propyl or isopropyl) and butyl (n-butyl, isobutyl, sec-butyl or tert-butyl). R_{5bc} may also be C₅alkylOH or C₆alkylOH. In a sixth embodiment, R_{5bc} is C₁₋₆haloalkyl such as C₁₋₄haloalkyl such as C₁haloalkyl (e.g. CF₃), C₂haloalkyl (e.g. CH₂CF₃), C₃haloalkyl (e.g. CH₂CH₂CF₃), C₄haloalkyl (e.g. CH₂CH₂CH₂CF₃). R_{5bc} may also be C₅haloalkyl (e.g. CH₂CH₂CH₂CH₂CF₃) or C₆haloalkyl (e.g. CH₂CH₂CH₂CH₂CH₂CF₃). In a seventh embodiment, R_{5bc} is C₀₋₂alkyleneC₃₋₆heterocycloalkyl such as C₀₋₂alkyleneC₃heterocycloalkyl, C₀₋₂alkyleneC₄heterocycloalkyl, C₀₋₂alkyleneC₅heterocycloalkyl, C₀₋₂alkyleneC₆heterocycloalkyl, C₀alkyleneC₃₋₆heterocycloalkyl, C₁alkyleneC₃₋₆heterocycloalkyl and C₂alkyleneC₃₋₆heterocycloalkyl. Suitably the heterocycloalkyl is a heterocyclopropyl, heterocyclobutyl, heterocyclopentyl or heterocyclohexyl ring such as a heterocyclohexyl ring. Suitably, the heterocyclopentyl ring is tetrahydrofuranyl or pyrrolidinyl. Suitably, the heterocyclohexyl ring is tetrahydropyranyl or piperidinyl. Any nitrogen atom such as one nitrogen in the C₃₋₆heterocycloalkyl ring may be substituted, for example by C₁₋₄alkyl, C(O)H, C(O)C₁₋₄alkyl, C(O)OC₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃alkylOC₁₋₂alkyl such as C(O)CH₂OCH₃, and C₁₋₂alkylC(O)OC₁₋₄alkyl such as CH₂C(O)OCH₂CH₃. Suitably, any nitrogen atom in the C₃₋₆heterocycloalkyl ring is not substituted.

Suitably R_{5bc} is H, CH₃ or ethyl, in particular CH₃ or ethyl. Suitably, R_{4bc} and R_{5bc} together with the carbon atom to which they are attached form a C₃₋₆cycloalkyl ring, such as a cyclopropyl ring.

Suitably R_{4bc} is H, CH₃ or ethyl and R_{5bc} is H, CH₃ or ethyl, in particular R_{4bc} is CH₃ or ethyl and R_{5bc} is CH₃ or ethyl. For example, R_{4bc} and R_{5bc} are H, R_{4bc} and R_{5bc} are methyl or R_{4bc} and R_{5bc} are ethyl.

Suitably, R_{4bc} is CH₂CH₂OCH₃ and R_{5bc} is H.

In one embodiment, A_{c} is A_{ac} i.e. —CH₂NR_{6c}—.

In another embodiment, A_{c} is A_{bc} i.e. —C(═O)NR_{6c}—.

In one embodiment, R_{6c} is H. In another embodiment, R_{6c} is C₁₋₃alkyl, in particular CH₃.

In one embodiment Ar1c is a 6-membered aryl, i.e. phenyl. In a second embodiment Ar1c is a 6-membered heteroaryl, in particular containing one nitrogen atom (pyridyl) or two nitrogen atoms (pyridazinyl, pyrimidinyl or pyrazinyl).

In particular Ar1c is phenyl or 2-pyridyl, such as phenyl.

In one embodiment R_{10c} is H. In a second embodiment R_{10c} is halo, for example fluoro or chloro. In a third embodiment R_{10c} is C₁₋₃alkyl, i.e. CH₃, ethyl or propyl (e.g. n-propyl or iso-propyl). In a fourth embodiment R_{10c} is OC₁₋₂alkyl, such as OCH₃ or ethoxy. In a fifth embodiment, R_{10c} is C₁₋₂ haloalkyl, such as CF₃ or CH₂CF₃. In a sixth embodiment R_{10c} is OC₁₋₂haloalkyl, such as OCF₃. In a seventh embodiment R_{10c} is CN.

Suitably R_{10c} is H, fluoro, OCH₃, CH₃ or CF₃, in particular H or fluoro, especially H.

Suitably R_{10c} is attached at the ortho position of Ar1c relative to group A_{c}(i.e. proximal to group A_{c}).

In one embodiment R_{11c} is H. In a second embodiment R_{11c} is F. In a third embodiment, R_{11c} is Cl. In a fourth embodiment R_{11c} is CH₃. In a fifth embodiment R_{11c} is CH₂CH₃. In a sixth embodiment R_{11c} is OCH₃. In a seventh embodiment R_{11c} is CF₃. In an eighth embodiment R_{11c} is OCF₃. In a ninth embodiment R_{11c} is CN.

In one embodiment, R_{11c} is in the ortho position relative to group A_{c}. In another embodiment R_{11c} is in the meta position relative to group A_{c}.

In one embodiment Ar2c is a 6-membered aryl, i.e. phenyl. In a second embodiment Ar2c is a 6-membered heteroaryl, in particular containing one nitrogen atom (pyridyl) or two nitrogen atoms (pyridazinyl, pyrimidinyl or pyrazinyl).

In particular Ar2c is 3-pyridyl or 2,5-pyrazinyl, especially 2,5-pyrazinyl.

In one embodiment R_{12c} is H. In a second embodiment R_{12c} is halo, for example fluoro or chloro. In a third embodiment R_{12c} is C₁₋₄alkyl, i.e. methyl, ethyl, propyl (n-propyl or isopropyl) or butyl (n-butyl, isobutyl, sec-butyl or tert-butyl). In a fourth embodiment, R_{12c} is C₂₋₄alkynyl such as C₂alkynyl (i.e. C≡CH). In a fifth embodiment, R_{12c} is C(═O)C₁₋₂alkyl, such as C(═O)C₁alkyl or C(═O)C₂alkyl. In a sixth embodiment R_{12c} is OC₁₋₂alkyleneC₃₋₅cycloalkyl, such as OC₃₋₅cycloalkyl (e.g. cyclopropoxy or cyclobutoxy), OC₁alkyleneC₃₋₅cycloalkyl or OC₂alkyleneC₃₋₅cycloalkyl. In a seventh embodiment R_{12c} is OC₁₋₄alkyl, such as OCH₃₋₅ethoxy, iso-propoxy or n-propoxy. In an eighth embodiment, R_{12c} is C₁₋₃alkyleneOC₁₋₃alkyl in particular C₁₋₂alkyleneOC₁₋₂alkyl such as C₁alkyleneOC₁alkyl, C₂alkyleneOC₁alkyl, C₁alkyleneOC₂alkyl or C₂alkyleneOC₂alkyl. In a ninth embodiment R_{12c} is C₁₋₄haloalkyl, such as CF₃. In a tenth embodiment R_{12c} is OC₁₋₄haloalkyl, such as OCF₃, OCHF₂ or OCH₂CF₃. In an eleventh embodiment R_{12c} is CN. In an eleventh embodiment R_{12c} is OC₀₋₂alkyleneC₃₋₅cycloalkyl, such as OC₃₋₅cycloalkyl (e.g. cyclopropoxy or cyclobutoxy), OC₁alkyleneC₃₋₅cycloalkyl or OC₂alkyleneC₃₋₅cycloalkyl. In a twelfth embodiment R_{12c} is OCH₂CH₂N(CH₃)₂. In a thirteenth embodiment R_{12c} is OH. In a fourteenth embodiment R_{12c} is C₁₋₄alkylOH, such as CH₂OH or C(CH₃)₂OH. In a fifteenth embodiment R_{12c} is NR_{23c}R_{24c}. In a sixteenth embodiment R_{12c} is SO₂CH₃. In a seventeenth embodiment R_{12c} is C(O)N(CH₃)₂. In an eighteenth embodiment R_{12c} is NHC(O)C₁₋₃alkyl such as NHC(O)CH₃. In a nineteenth embodiment R_{12c} is a C₃₋₆heterocycloalkyl comprising (such as containing) one nitrogen located at the point of attachment to Ar2c, such as a C₅heterocycloalkyl, in particular pyrrolidinyl, or a C₆heterocycloalkyl such as morpholinyl. In a twentieth embodiment, R_{12c} together with a nitrogen atom to which it is attached forms an N-oxide (N⁺—O⁻).

In one embodiment, R_{23c} is H. In another embodiment, R_{23c} is C₁₋₂alkyl i.e. CH₃ or CH₂CH₃.

In one embodiment, R_{24c} is H. In another embodiment, R_{24c} is C₁₋₂alkyl i.e. CH₃ or CH₂CH₃.

R_{12c} is suitably H, fluoro, chloro, CH₃, Et, OCH₃, OEt, OiPr, CF₃ or OCH₂CF₃. In particular, R_{12c} is fluoro, chloro, CH₃, OCH₃, OEt, OiPr or CF₃, for example chloro, OEt, OiPr or CF₃ such as chloro, OEt or CF₃.

R_{12c} is suitably attached at the meta position of Ar2c. Alternatively, R_{12c} is attached at the ortho position of Ar2c.

The present invention provides compounds R94 and R95.

The present invention provides the following compounds:
N-(4-(2-((4-(6-Ethoxypyrazin-2-yl)-2-fluorobenzyl)amino)propan-2-yl)thiazol-2-yl)cyclopropanesulfonamide; and
N-(4-(2-(((5-(6-ethoxypyrazin-2-yl)pyridin-2-yl)methyl)amino)propan-2-yl)thiazol-2-yl)cyclopropanesulfonamide.

### General Terms and Definitions

In this section reference to variables and compounds of formula (I) are taken to generically cover variables wherein the suffix “a”, “b” or “c” has been added. For example, reference to R₄ is taken to include reference to R₄ₐ, R_{4b} and R_{4c} as well as sub-groups within these variables. The same applies for all other variables discussed in this section. Similarly, reference to compounds of formula (I) is taken to include compounds of formula (I-a), (I-b) and/or (I-c).

The term ‘alkyl’ as used herein, such as in C₁₋₃alkyl, C₁₋₄alkyl, C₁₋₅alkyl or C₁₋₆alkyl e.g. C₁₋₃alkyl, C₁₋₄alkyl or C₁₋₅alkyl, or such as in C₁₋₂alkyl, C₁₋₃alkyl or C₁₋₄alkyl whether alone or forming part of a larger group such as an Oalkyl group (e.g. OC₁₋₃alkyl, OC₁₋₄alkyl and OC₁₋₅alkyl or OC₁₋₂alkyl, OC₁₋₃alkyl or OC₁₋₄alkyl), is a straight or a branched fully saturated hydrocarbon chain containing the specified number of carbon atoms. Examples of alkyl groups include the C₁₋₅alkyl groups methyl, ethyl, n-propyl, iso-propyl, n-butyl, iso-butyl, sec-butyl, tert-butyl and n-pentyl, sec-pentyl and 3-pentyl, in particular the C₁₋₃alkyl groups methyl, ethyl, n-propyl and iso-propyl. Examples of alkyl groups also include the C₁₋₄alkyl groups methyl, ethyl, n-propyl, iso-propyl, n-butyl, iso-butyl, sec-butyl and tert-butyl, in particular the C₁₋₃alkyl groups methyl, ethyl, n-propyl and iso-propyl such as C₁₋₂alkyl groups methyl and ethyl. Reference to “propyl” includes n-propyl and iso-propyl, and reference to “butyl” includes n-butyl, isobutyl, sec-butyl and tert-butyl. Examples of Oalkyl groups include the OC₁₋₄alkyl groups methoxy, ethoxy, propoxy (which includes n-propoxy and iso-propoxy) and butoxy (which includes n-butoxy, iso-butoxy, sec-butoxy and tert-butoxy). C₅alkyl groups as used herein, whether alone or forming part of a larger group such as an OC₅alkyl group is a straight or a branched fully saturated hydrocarbon chain containing five carbon atoms. Examples of C₅alkyl groups include n-pentyl, sec-pentyl, 3-pentyl, sec-isopentyl and active pentyl. C₅alkyl groups as used herein, whether alone or forming part of a larger group such as an OC₆alkyl group is a straight or a branched fully saturated hydrocarbon chain containing six carbon atoms. Examples of C₆alkyl groups include n-hexyl, 2-methylpentyl, 3-methylpentyl, 2,2-dimethylbutyl and 2,3-dimethylbutyl.

The term ‘alkylene’ as used herein, such as in C₀₋₂alkyleneC₃₋₅cycloalkyl, C₁₋₃alkyleneOC₁₋₂alkyl C₁₋₂alkyleneOC₁₋₂alkyl or OC₀₋₂alkyleneC₃₋₅cycloalkyl is a bifunctional straight or a branched fully saturated hydrocarbon chain containing the specified number of carbon atoms. Examples of C₀₋₂alkylene groups are where the group is absent (i.e. C₀), methylene (C₁) and ethylene (C₂). Examples of C₁₋₃alkylene groups are where the group is methylene (C₁), ethylene (C₂) and propylene (C₃). Examples of C₁₋₂alkylene groups are where the group is methylene (C₁) and ethylene (C₂). Examples of C₀₋₁alkylene groups are where the group is absent (C₀) and methylene (C₁).

The term ‘alkenyl’ as used herein, such as in C₂₋₄alkenyl, is a straight or branched hydrocarbon chain containing the specified number of carbon atoms and a carbon-carbon double bond.

The term ‘alkynyl’ as used herein, such as in C₂₋₄alkynyl such as in C₂alkynyl is an unbranched hydrocarbon chain containing the specified number of carbons (e.g. 2, 3 or 4 carbons, such as two carbons), two of which carbon atoms are linked by a carbon-carbon triple bond.

The term ‘cycloalkyl’ as used herein, such as in C₃₋₄cycloalkyl, C₃₋₅cycloalkyl or C₃₋₆cycloalkyl, whether alone or forming part of a larger group such as OC₃₋₅cycloalkyl, C₀₋₂alkyleneC₃₋₆cycloalkyl, C₀₋₂alkyleneC₃₋₅cycloalkyl or C₀₋₁alkyleneC₃₋₄cycloalkyl is a fully saturated hydrocarbon ring containing the specified number of carbon atoms. Examples of cycloalkyl groups include the C₃₋₆cycloalkyl groups cyclopropyl, cyclobutyl, cyclopentyl and cyclohexyl, in particular the C₃₋₅cycloalkyl groups cyclopropyl, cyclobutyl and cyclopentyl:

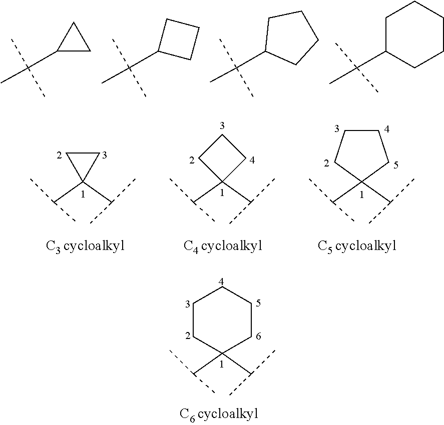

When B is B-a, the term ‘heterocycloalkyl’ as used herein, such as in C₃₋₆heterocycloalkyl or C₀₋₂alkyleneC₃₋₆heterocycloalkyl is a fully saturated hydrocarbon ring containing the specified number of carbon atoms, wherein at least one of the carbon atoms in the ring is replaced by a heteroatom such as N, S or O. When B is B-bc, the term ‘heterocycloalkyl’ as used herein, such as in C₃₋₆heterocycloalkyl or C₀₋₂alkyleneC₃₋₆heterocycloalkyl is a fully saturated hydrocarbon ring containing the specified number of ring atoms and includes the ring atom through which the heterocycloalkyl group is attached, wherein at least one of the atoms in the ring is a heteroatom such as O, N or S.

As required by valency, the nitrogen atom(s) may be connected to a hydrogen atom to form an NH group. Alternatively the nitrogen atom(s) may be substituted (such as one nitrogen atom is substituted), for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃alkylOC₁₋₂alkyl such as C(O)CH₂OCH₃, and C₁₋₂alkylC(O)OC₁₋₄alkyl such as CH₂C(O)OCH₂CH₃. Wherein a ring heteroatom is S, the term ‘heterocycloalkyl’ includes wherein the S atom(s) is substituted (such as one S atom is substituted) by one or two oxygen atoms (i.e. S(O) or S(O)₂). Alternatively, any sulphur atom(s) in the C₃₋₆heterocycloalkyl ring is not substituted.

Examples of C₃₋₆heterocycloalkyl groups include those comprising one heteroatom such as containing one heteroatom (e.g. oxygen) or containing two heteroatoms (e.g. two oxygen atoms or one oxygen atom and one nitrogen atom). Other examples of C₃₋₆heterocycloalkyl include those comprising one heteroatom atom such as containing one heteroatom (e.g. one oxygen atom or one nitrogen atom) or containing two heteroatoms (e.g. two nitrogen atoms or one nitrogen atom and one oxygen atom). Particular examples of C₃₋₆heterocycloalkyl comprising one nitrogen atom include pyrrolidinyl, pyrazolidinyl, piperidinyl, piperazinyl, morpholinyl and thiomorpholinyl, especially piperidinyl, pyrrolidinyl and morpholinyl. Particular examples of C₃₋₆heterocycloalkyl comprising one oxygen atom include oxiranyl, oxetanyl, 3-dioxolanyl, morpholinyl, 1,4-oxathianyl, tetrahydropyranyl, 1,4-thioxanyl and 1,3,5-trioxanyl. Examples of C₃₋₆heterocycloalkyl include those comprising one oxygen atom such as containing one oxygen atom, or containing two oxygen atoms. Particular examples of C₃₋₆heterocycloalkyl comprising one oxygen atom include oxiranyl, oxetanyl, 3-dioxolanyl, morpholinyl, 1,4-oxathianyl, tetrahydropyranyl, 1,4-thioxanyl and 1,3,5-trioxanyl. Particular examples of C₃₋₆heterocycloalkyl comprising one nitrogen atom include piperidinyl. Other examples of C₃₋₆heterocycloalkyl comprising one nitrogen atom include pyrrolidinyl, pyrazolidinyl, imidazolidinyl, thiazolidinyl, piperidinyl, piperazinyl, morpholinyl and thiomorpholinyl, such as piperidinyl.

In one embodiment and when B is B-a, the term ‘heterocycloalkyl’ as used herein, such as in C₃₋₆heterocycloalkyl is a fully saturated hydrocarbon ring containing the specified number of carbon atoms, wherein at least one of the carbon atoms in the ring is replaced by a heteroatom such as N, S or O. Examples of C₃₋₆heterocycloalkyl groups include those comprising one heteroatom such as containing one heteroatom (e.g. oxygen) or containing two heteroatoms (e.g. two oxygen atoms or one oxygen atom and one nitrogen atom).

When B is B-bc and the compound is a compound of formula (I-b), the term ‘heterocycloalkyl’ as used herein, such as in C₃₋₆heterocycloalkyl is a fully saturated hydrocarbon ring containing the specified number of ring atoms and includes the ring atom through which the heterocycloalkyl group is attached, wherein at least one of the atoms in the ring is a heteroatom such as O, N or S. Examples of C₃₋₆heterocycloalkyl include those comprising one nitrogen atom such as containing one heteroatom (i.e. nitrogen) or containing two heteroatoms (e.g. two nitrogen atoms or one nitrogen atom and one oxygen atom).

The term ‘5- or 6-membered oxygen-containing heterocycloalkyl’ as used herein, is a fully saturated hydrocarbon ring containing the specified number of ring atoms (i.e. 5 or 6), wherein at least one ring atom is an oxygen atom and the ring does not contain heteroatoms other than oxygen. Examples of oxygen-containing heterocycloalkyl groups are oxiranyl, oxetanyl, tetrahydrofuranyl, 3-dioxolanyl, tetrahydropyranyl, and 1,3,5-trioxanyl, such as tetrahydrofuranyl and tetrahydropyranyl. An example of a nitrogen-containing heterocycloalkyl group is piperidinyl.

The heterocycloalkyl groups may have any one of the following structures:

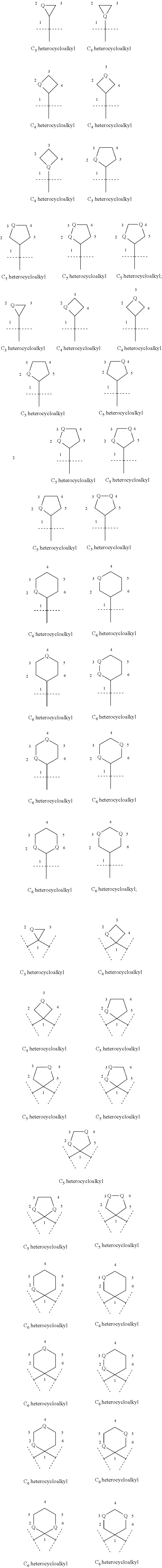

wherein each Q is independently selected from O, N or S, such as O or N. When Q is N, as required by valency, the nitrogen atom(s) may be connected to a hydrogen atom to form an NH group. Alternatively the nitrogen atom(s) may be substituted (such as one nitrogen atom is substituted), for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃alkylOC₁₋₂alkyl such as C(O)CH₂OCH₃, and C₁₋₂alkylC(O)OC₁₋₄alkyl such as CH₂C(O)OCH₂CH₃. When any Q is S, the S atoms can be substituted (such as one S atom is substituted) by one or two oxygen atoms (i.e. S(O) or S(O)₂). When R₄ and R₅ are R₄ₐ and R₅ₐ, Q is N substituted by S(O)₂R₂₉. Alternatively, any sulphur atom(s) in the C₃₋₆heterocycloalkyl ring is not substituted.

When Aₐ is —C(═O)NH—, —NH— or —CH₂NH— and R₄ₐ and/or R₅ₐ is C₀alkyleneC₃₋₆heterocycloalkyl, or when R₄ₐ and R₅ₐ together with the carbon atom to which they are attached form a C₃₋₆heterocycloalkyl, any heteroatom in the heterocycloalkyl may not be directly connected to the carbon to which R₄ₐ and R₅ₐ are connected.

When B is B-a, suitably, heterocycloalkyl is a fully saturated hydrocarbon ring containing the specified number of carbon atoms wherein at least one of the carbon atoms is replaced by a heteroatom such as N, S or O wherein as required by valency, any nitrogen atom is connected to a hydrogen atom, and wherein the S atom is not present as an oxide.

When B is B-bc and the compound is a compound of formula (I-b), suitably, heterocycloalkyl is a fully saturated hydrocarbon ring containing the specified number of ring atoms and includes the ring atom through which the heterocycloalkyl group is attached, wherein at least one of the atoms in the ring is a heteroatom such as O, N or S. Suitably, as required by valency, any nitrogen atom is connected to a hydrogen atom. Suitably any S atom is not present as an oxide. In particular, any nitrogen atom is connected to a hydrogen atom and any S atom is not present as an oxide.

When B is B-bc and the compound is a compound of formula (I-c), and when the heterocycloalkyl is formed from R₄ and R₅ together with the carbon atom to which they are attached, suitably any heteroatom is not directly connected to the carbon to which R₄ and R₅ are attached. Thus suitably, when the heterocycloalkyl is formed from R₄ and R₅ together with the carbon atom to which they are attached, the heterocycloalkyl may be:

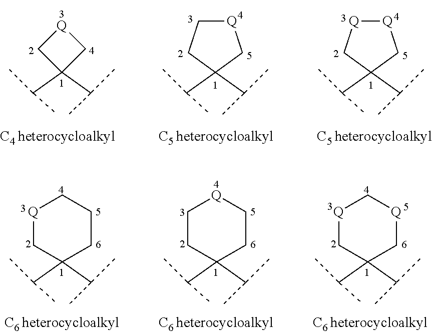

wherein each Q is independently O, N or S such as O or N. When Q is N, as required by valency, the nitrogen atom(s) may be connected to a hydrogen atom to form an NH group. Alternatively the nitrogen atom (s) may be substituted (such as one nitrogen atom is substituted), for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄haloalkyl such as C(O)OtBu. Additional substituent groups on any nitrogen atom(s) in the C₃₋₆heterocycloalkyl ring include C₁₋₄alkylCN such as CH₂CN, C(O)C₁₋₃ alkylOC₁₋₂alkyl such as C(O)CH₂OCH₃, and C₁₋₂alkylC(O)OC₁₋₄alkyl such as CH₂C(O)OCH₂CH₃. When any Q is S, the S atom(s) can be substituted (such as one S atom is substituted) by one or two oxygen atoms (i.e. S(O) or S(O)₂). When R₄ and R₅ are R₄ₐ and R₅ₐ, Q is N substituted by —S(O)₂R₂₉. Alternatively, any sulphur atom(s) in the C₃₋₆heterocycloalkyl ring is not substituted.

The term ‘halo’ or ‘halogen’ as used herein, refers to fluorine, chlorine, bromine or iodine. Particular examples of halo are fluorine and chlorine, especially fluorine.

The term ‘haloalkyl’ as used herein, such as in C₁₋₆haloalkyl, such as in C₁₋₄haloalkyl or C₁₋₂ haloalkyl, whether alone or forming part of a larger group such as an Ohaloalkyl group, such as in OC₁₋₆haloalkyl, such as in OC₁₋₄haloalkyl or OC₁₋₂haloalkyl, is a straight or a branched fully saturated hydrocarbon chain containing the specified number of carbon atoms and at least one halogen atom, such as fluoro or chloro, especially fluoro. An example of haloalkyl is CF₃. Further examples of haloalkyl are CHF₂ and CH₂CF₃. Another example of haloalkyl is CH₂CHF₂. Examples of Ohaloalkyl include OCF₃, OCHF₂ and OCH₂CF₃.

The term ‘6-membered aryl’ as used herein refers to a phenyl ring.

The term ‘6-membered heteroaryl’ as used herein refers to 6-membered aromatic rings containing at least one heteroatom (e.g. nitrogen). Exemplary 6-membered heteroaryls include one nitrogen atom (pyridinyl), two nitrogen atoms (pyridazinyl, pyrimidinyl or pyrazinyl) and three nitrogen atoms (triazinyl).

The phrase ‘in the para position relative to group A’ as used herein, such as in relation to the position of Ar2, means that compounds with the following substructure are formed:

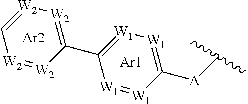

wherein when B is B-a, W₁ may be N, CH, CR₁₀ₐ or CR₁₁ₐ, and W₂ may be N, CH or CR₁₂ₐ as allowed by the definitions provided for compounds of formula (I). W₂ may also be CR₁₃ₐ as allowed by the definitions provided for compounds of formula (I-a); and

when B is B-bc and the compound is a compound of formula (I-b), compounds with the following substructure are formed:

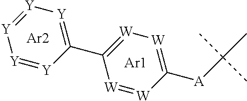

wherein W may be N, CH, CR_{10b} or CR_{11b}, and Y may be N, CH, CR_{12b} or CR_{13b} as allowed by the definitions provided for compounds of formula (I-b);

and when B is B-bc and the compound is a compound of formula (I-c), compounds with the following substructure are formed:

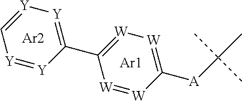

wherein W may be N, CH or CR_{10c}, and Y may be N, CH or CR_{12c} as required by the definitions provided for compounds of formula (I-c). W may also be CR_{11c} as allowed by the definitions provided for compounds of formula (I-c).

The terms ‘ortho’ and ‘meta’ as used herein, such as when used in respect of defining the position of R₁₂ on Ar2 is with respect to Ar1, means that the following structures may form:

when B is B-a:

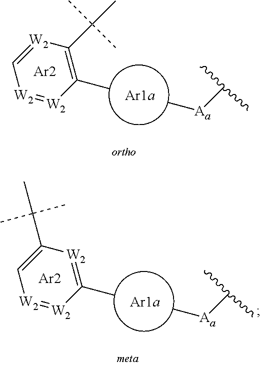

when B is B-bc and the compound is a compound of formula (I-b):

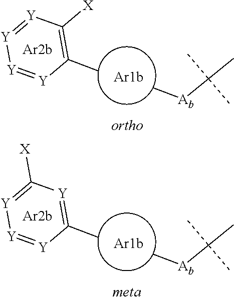

wherein X represents a substituent e.g. R_{12b}; and

when B is B-bc and the compound is a compound of formula (I-c):

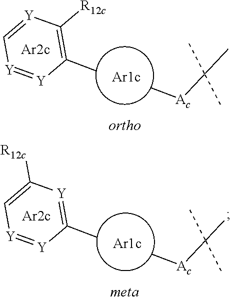

wherein all variables listed above are as defined elsewhere herein.

When a spirocyclic ring system is said to form, e.g. when R₄ₐₐ and R₅ₐₐ, R_{4ab} and R_{5ab}, or R_{4ac} and R_{5ac}, suitably together with the carbon atom to which they are attached form a C₃₋₆cycloalkyl and one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, the following spirocyclic groups are encompassed (which may optionally be substituted as mentioned above):

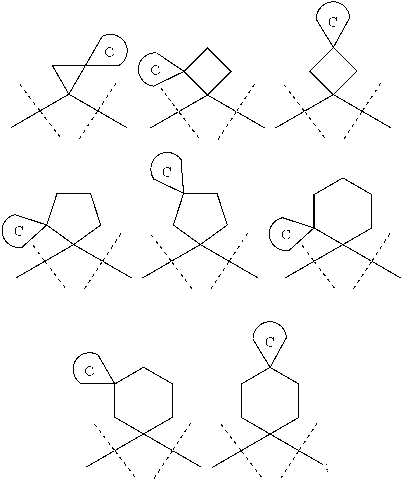

wherein C is a C₃₋₆cycloalkyl ring or a C₃₋₆heterocycloalkyl ring, as defined elsewhere herein. In one embodiment C is a C₃₋₆cycloalkyl ring. In a second embodiment C is a C₃₋₆heterocycloalkyl ring.

For example, one of the carbons is quaternary and is attached to a 5-membered dioxalane ring to form the following structure:

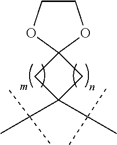

wherein m is 1 or 2 and n is 0, 1 or 2. Suitably m is 2 and n is 2.

Alternatively, when a spirocyclic ring system is said to form, e.g. when R₄ₐₐ and R₅ₐₐ, R_{4ab} and R_{5ab}, or R_{4ac} and R_{5ac} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, the following spirocyclic groups are encompassed (which may optionally be substituted as mentioned above):

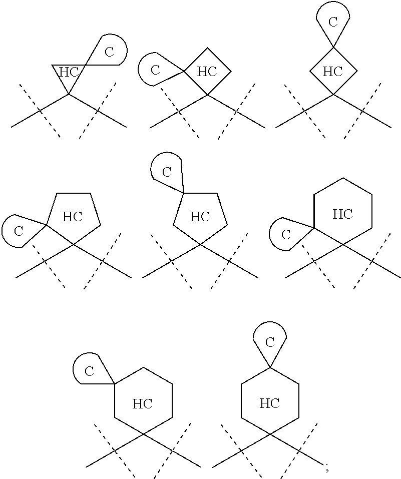

wherein C is a C₃₋₆cycloalkyl ring or a C₃₋₆heterocycloalkyl ring, as defined elsewhere herein, and HC is a C₃₋₆heterocycloalkyl ring formed by R₄ₐ and R₅ₐ (i.e. R₄ₐₐ and R₅ₐₐ, R_{4ab} and R_{ab}, or R_{4ac} and R_{5ac}) as defined elsewhere herein. In one embodiment C is a C₃₋₆cycloalkyl ring. In a second embodiment C is a C₃₋₆heterocycloalkyl ring.

Throughout the specification Ar1 and Ar2 may be depicted as follows:

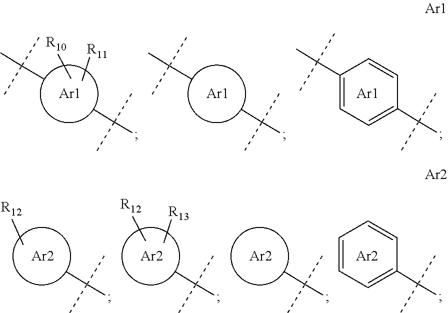

All depictions with respect to Ar1 are equivalent and all depictions with respect to Ar2 are equivalent, unless the context requires otherwise, depictions of Ar1 and Ar2 should not be taken to exclude the presence of heteroatoms or substitutions. Ar1 encompasses the variables Ar1 a, Ar1b and Ar1c. Ar2 encompasses the variables Ar2a, Ar2b and Ar2c.

The present invention provides N-oxides of the compound of formula (I). Suitably, when R₁₂ together with a nitrogen atom to which it is attached forms an N-oxide (N⁺—O⁻), the example following structures are formed:

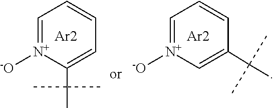

The position numbering for Ar1 is in respect of group A, with the carbon at the point of attachment designated position 1 and other numbers providing the relative location of the nitrogen atoms, for example:

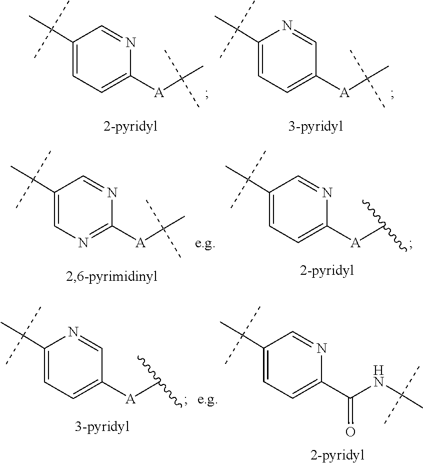

The position numbering for Ar2 is in respect of the point of attachment to Ar1, for example:

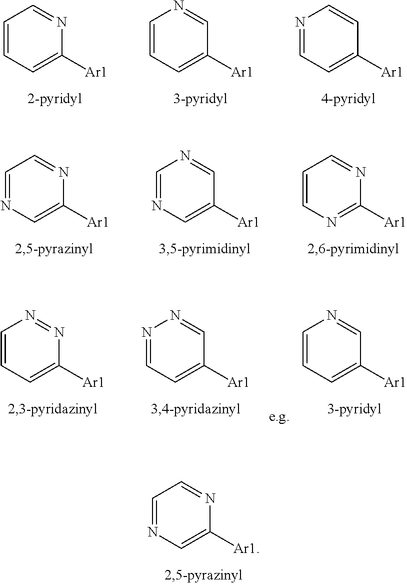

Reference to compounds of formula (I) throughout the application is intended to encompass reference to compounds of formulae (I-a), (I-b) and (I-c).

The compounds of the invention may be provided in the form of a pharmaceutically acceptable salt and/or solvate thereof and/or derivative thereof. In particular, the compound of formula (I) may be provided in the form of a pharmaceutically acceptable salt and/or solvate, such as a pharmaceutically acceptable salt Compounds of the invention of particular interest are those demonstrating an IC₅₀ of 1 uM or lower, especially 100 nM or lower, in respect of CTPS1 enzyme, using the methods of the examples (or comparable methods).

Compounds of the invention of particular interest are those demonstrating a selectivity for CTPS1 over CTPS2 of 2-30 fold, suitably >30-60 fold or more suitably >60 fold, using the methods of the examples (or comparable methods). Desirably the selectivity is for human CTPS1 over human CTPS2.

It will be appreciated that for use in medicine the salts of the compounds of formula (I) should be pharmaceutically acceptable. Non-pharmaceutically acceptable salts of the compounds of formula (I) may be of use in other contexts such as during preparation of the compounds of formula (I). Suitable pharmaceutically acceptable salts will be apparent to those skilled in the art.

Pharmaceutically acceptable salts include those described by Berge et al. (1977). Such pharmaceutically acceptable salts include acid and base addition salts. Pharmaceutically acceptable acid additional salts may be formed with inorganic acids e.g. hydrochloric, hydrobromic, sulphuric, nitric or phosphoric acid and organic acids e.g. succinic, maleic, acetic, fumaric, citric, tartaric, benzoic, p-toluenesulfonic, methanesulfonic or naphthalenesulfonic acid. Other salts e.g. oxalates or formates, may be used, for example in the isolation of compounds of formula (I) and are included within the scope of this invention.

Certain of the compounds of formula (I) may form acid or base addition salts with one or more equivalents of the acid or base. The present invention includes within its scope all possible stoichiometric and non-stoichiometric forms.

The compounds of formula (I) may be prepared in crystalline or non-crystalline form and, if crystalline, may optionally be solvated, e.g. as the hydrate. This invention includes within its scope stoichiometric solvates (e.g. hydrates) as well as compounds containing variable amounts of solvent (e.g. water).

It will be understood that the invention includes pharmaceutically acceptable derivatives of compounds of formula (I) and that these are included within the scope of the invention.

As used herein “pharmaceutically acceptable derivative” includes any pharmaceutically acceptable prodrug such as an ester or salt of such ester of a compound of formula (I) which, upon administration to the recipient is capable of providing (directly or indirectly) a compound of formula (I) or an active metabolite or residue thereof.

It is to be understood that the present invention encompasses all isomers of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses.

The present disclosure includes all isotopic forms of the compounds of the invention provided herein, whether in a form (i) wherein all atoms of a given atomic number have a mass number (or mixture of mass numbers) which predominates in nature (referred to herein as the “natural isotopic form”) or (ii) wherein one or more atoms are replaced by atoms having the same atomic number, but a mass number different from the mass number of atoms which predominates in nature (referred to herein as an “unnatural variant isotopic form”). It is understood that an atom may naturally exist as a mixture of mass numbers. The term “unnatural variant isotopic form” also includes embodiments in which the proportion of an atom of given atomic number having a mass number found less commonly in nature (referred to herein as an “uncommon isotope”) has been increased relative to that which is naturally occurring e.g. to the level of >20%, >50%, >75%, >90%, >95% or >99% by number of the atoms of that atomic number (the latter embodiment referred to as an “isotopically enriched variant form”). The term “unnatural variant isotopic form” also includes embodiments in which the proportion of an uncommon isotope has been reduced relative to that which is naturally occurring. Isotopic forms may include radioactive forms (i.e. they incorporate radioisotopes) and non-radioactive forms. Radioactive forms will typically be isotopically enriched variant forms.

An unnatural variant isotopic form of a compound may thus contain one or more artificial or uncommon isotopes such as deuterium (²H or D), carbon-11 (¹¹C), carbon-13 (¹³C), carbon-14 (¹⁴C), nitrogen-13 (¹³N), nitrogen-15 (¹⁵N), oxygen-15 (¹⁵O), oxygen-17 (¹⁷O), oxygen-18 (¹⁹O), phosphorus-32 (³²P), sulphur-35 (³⁵S), chlorine-36 (³⁶Cl), chlorine-37 (³⁷Cl), fluorine-18 (¹⁸F) iodine-123 (¹²³I), iodine-125 (¹²⁵I) in one or more atoms or may contain an increased proportion of said isotopes as compared with the proportion that predominates in nature in one or more atoms.

Unnatural variant isotopic forms comprising radioisotopes may, for example, be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Unnatural variant isotopic forms which incorporate deuterium i.e. ²H or D may afford certain therapeutic advantages resulting from greater metabolic stability, for example, increased in vivo half-life or reduced dosage requirements, and hence may be preferred in some circumstances. Further, unnatural variant isotopic forms may be prepared which incorporate positron emitting isotopes, such as ¹¹C, ¹⁸F, ¹⁵O and ¹³N, and would be useful in Positron Emission Topography (PET) studies for examining substrate receptor occupancy.

In one embodiment, the compounds of the invention are provided in a natural isotopic form.

In one embodiment, the compounds of the invention are provided in an unnatural variant isotopic form. In a specific embodiment, the unnatural variant isotopic form is a form in which deuterium (i.e. ²H or D) is incorporated where hydrogen is specified in the chemical structure in one or more atoms of a compound of the invention. In one embodiment, the atoms of the compounds of the invention are in an isotopic form which is not radioactive. In one embodiment, one or more atoms of the compounds of the invention are in an isotopic form which is radioactive. Suitably radioactive isotopes are stable isotopes. Suitably the unnatural variant isotopic form is a pharmaceutically acceptable form.

In one embodiment, a compound of the invention is provided whereby a single atom of the compound exists in an unnatural variant isotopic form. In another embodiment, a compound of the invention is provided whereby two or more atoms exist in an unnatural variant isotopic form.

Unnatural isotopic variant forms can generally be prepared by conventional techniques known to those skilled in the art or by processes described herein e.g. processes analogous to those described in the accompanying Examples for preparing natural isotopic forms. Thus, unnatural isotopic variant forms could be prepared by using appropriate isotopically variant (or labelled) reagents in place of the normal reagents employed in the Examples. Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

In general, the compounds of formula (I) may be made according to the organic synthesis techniques known to those skilled in this field, as well as by the representative methods set forth below, those in the Examples, and modifications thereof.

General Routes:

In this section reference to variables and compounds of formula (I) are taken to generically cover variables wherein the suffix “a”, “b” or “c” has been added. For example, reference to R₄ is taken to include reference to R₄ₐ, R_{4b} and R_{4c} as well as sub-groups within these variables. The same applies for all other variables discussed in this section. Reference to compounds of formula (I) is taken to include compounds of formula (I-a), (I-b) and/or (I-c).

Generic and specific routes by which compounds disclosed herein and compound examples of the invention may be conveniently prepared are disclosed in WO2019/106156, WO2019/106146, WO2019/179652, WO02019/180244 and WO2020/083975, each of which is herein incorporated in its entirety by reference, as well as those routes summarised below, and adaptations thereof.

Compounds of Formula (I-a)

Compounds of formula (I-a) may be synthesised according to general and specific methods disclosed in WO2019/179652, WO2019/180244 and WO02020/083975. For the avoidance of doubt, reference to variables in each of the schemes in this section encompasses variables specific to compounds of formula (I-a). For example, R₁ includes R₁ₐ. Furthermore, reference to intermediates in each of the schemes in this section encompasses corresponding intermediates for compounds of formula (I-a). For example, reference to compounds of formula (II) encompasses compounds of formula (II-a).

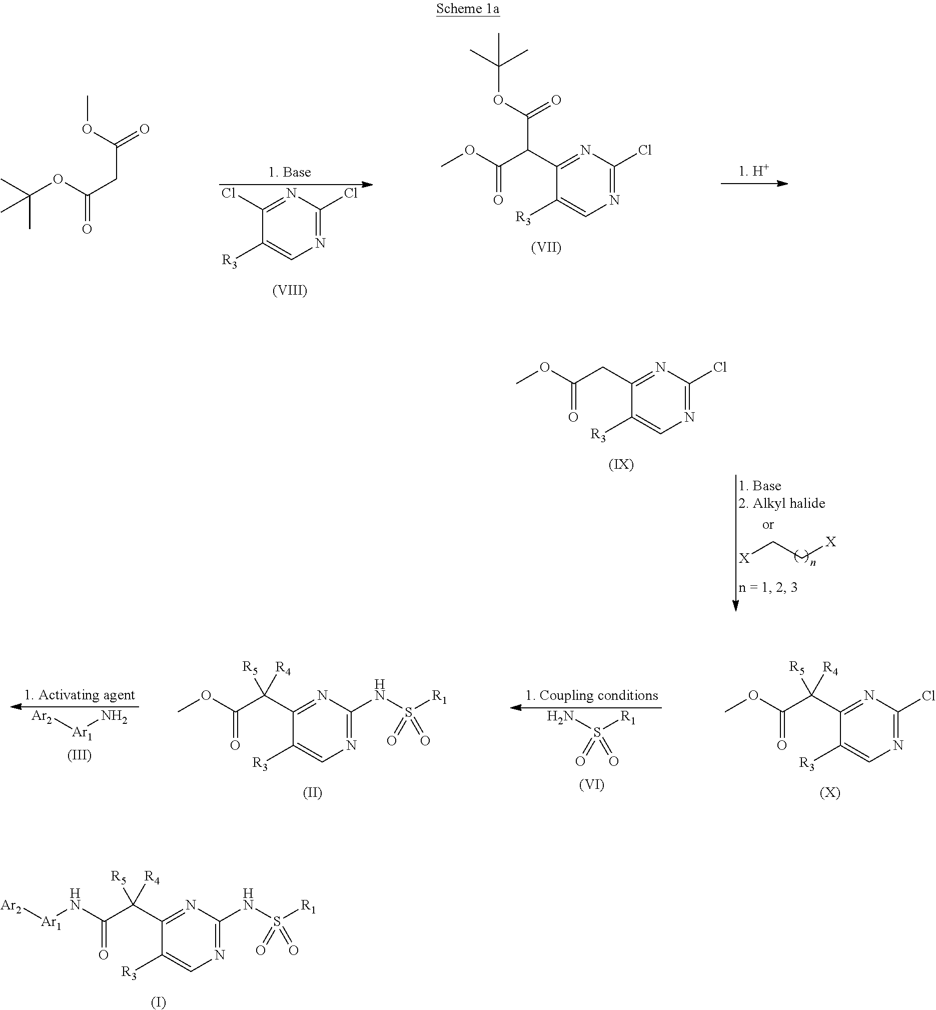

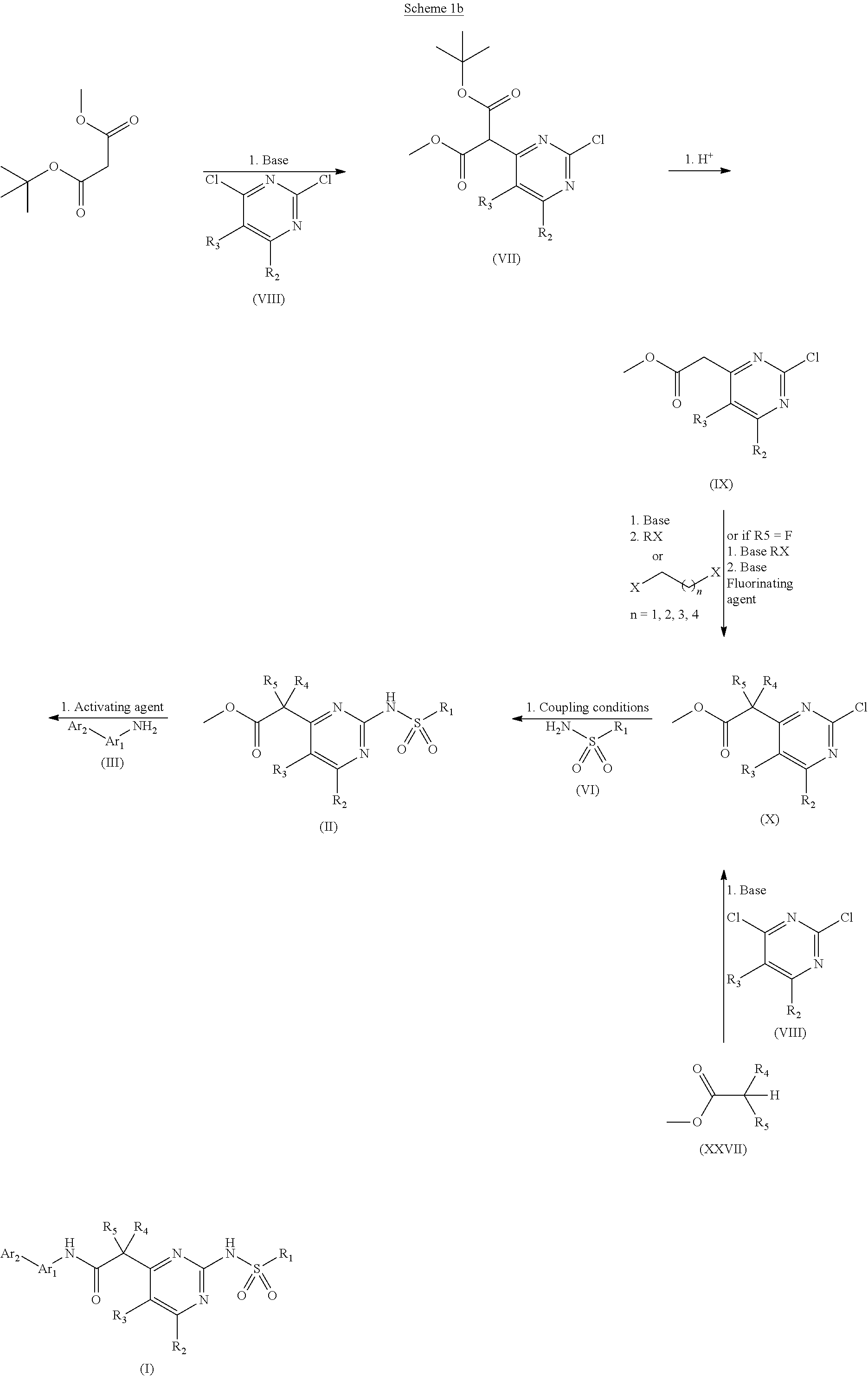

Suitably, R₂ is H, (IX) is converted to (X) using a base and alkyl halide or X—CH₂—(CH₂)n-X wherein n=1,2,3 and the compounds of general formula (I) are obtained by a five step process.

In general and as illustrated in Schemes 1a and 1b, compounds of general formula (I) may be obtained by a five or six step process from a 2,4-dichloropyrimidine derivative of general formula (VIII). Firstly, the derivative (VIII) can be reacted with an unsymmetrical malonate ester as shown in Schemes 1a, 1b of WO2019/179652, or 1a or 1b herein. For example, the unsymmetrical malonate ester can be treated with a base such as Cs₂CO_{M} in the presence of di-chloropyrimidine (VIII) in a solvent such as DMF and heated to an elevated temperature such as 80° C., followed by an aqueous work-up to obtain compounds of formula (VII). This intermediate compound can then be deprotected at this stage via a decarboxylation, initiated by the use of a strong acid such as TFA to yield intermediate derivative (IX). Certain intermediates such as (IX) where R₃=H, are commercially available. Reaction of a methyl 2-(2-chloropyrimidin-4-yl)acetate derivative of general formula (IX) with an inorganic base such as potassium carbonate, in the presence of an alkylating agent leads to alkylation alpha to the ester. It will be understood by persons skilled in the art that both mono- and dialkylation may be achieved with careful control of the reaction conditions, but for a more reliable synthesis of the monoalkylated product, an alternative procedure should be considered (as in Scheme 1a of WO2019179652). R₄ and R₅ can be connected to form a C₃₋₆cycloalkyl ring as defined above ((IX) to (X)). Such compounds may be prepared by double alkylation with a dihaloalkane, such as 1,2-dibromoethane or 1,3-dibromobutane in the presence of an inorganic base such as sodium hydroxide. For compounds of general formula (I) wherein R₄ and R₅ together with the carbon to which they are attached form a C₃₋₆heterocycloalkyl, double alkylation of intermediates (IX) using a di-haloheteroalkane (such as BrCH₂CH₂OCH₂CH₂Br) in the presence of a base such as Cs₂CO₃ in a solvent such as MeCN at an elevated temperature such as 60° C. followed by direct column chromatography can be used to provide compounds of formula (X).

Palladium catalysed sulfamination of intermediate (X) may be achieved using a catalyst such as [t-BuXPhosPd(allyl)]OTf or t-BuXPhos-Pd-G3 and substituted sulfonamide nucleophile (VI), in the presence of an inorganic base, for example potassium carbonate to form intermediate derivative (II). Alternatively, sulfamination of intermediate (X) may be achieved using a substituted sulfonamide nucleophile (VI), in the presence of an inorganic base, for example Cs₂CO₃ and a solvent such as N-methyl pyrrolidinone to form intermediates (II) which may be obtained by precipitation following dilution in aqueous 4M HCl.

Final transformation to compounds of general formula (I) can be prepared by conversion of intermediate (II) by activation of the ester moiety using trimethylaluminium (usually a 2.0 M solution in toluene or heptane) and addition of amine (ill) (commercially available or prepared as in Schemes 6a, 6b, 7a or 7b of WO2019179652). Alternatively, compounds of formula (I) may be obtained by a strong base-mediated amide formation between compounds (II) and (III) at room temperature using bases such as iPrMgCl, UHMDS or KOtBu.

Compounds of the general formula (VII) where R₂ is O-alkyl may be accessed in two steps from commercial 2,4,6-trichloropyrimidine derivatives such as (VIII) where R₂ is Cl. Reaction of an unsymmetrical malonate ester can yield compounds such as (VII) which can then be treated with an alkoxide base such as sodium methoxide to displace the more reactive chloride to give compounds of general formula (VII) where R₂=O-alkyl. Such compounds can then be progressed to final compounds of formula (I) following the steps previously described in Schemes 1a or 1b.

Compounds of general formula (I) where R1, Ar1 and Ar2 are defined above and R₄ and R₅ together with the carbon to which they are attached form a C₃₋₆heterocycloalkyl, may be prepared in five steps starting from intermediate of general formula (VIII). Firstly, alkyl esters of general formula (XXVII) can be treated with a strong base such as LIHMDS then reacted with 2,4-dichloropyrimidines such as derivative (VIII). Such compounds can then be converted to final compounds using the methods described in Scheme 1b. If any protecting groups remain after amide coupling, treatment with a strong acid such as TFA may yield final compounds of formula (I).

Following deprotection compounds of general formula (I) where R₁, Ar1 and Ar2 are defined above and R₄ and R₅ together with the carbon to which they are attached form a C₃₋₆aminocycloalkyl, may be further elaborated by treatment with a suitable electrophile such as an acid chloride or an isocyanate, to yield the corresponding amide or urea. Such compounds may also undergo reductive amination in the presence of a suitable aldehyde or ketone followed by treatment with sodium triacetoxyborohydride.

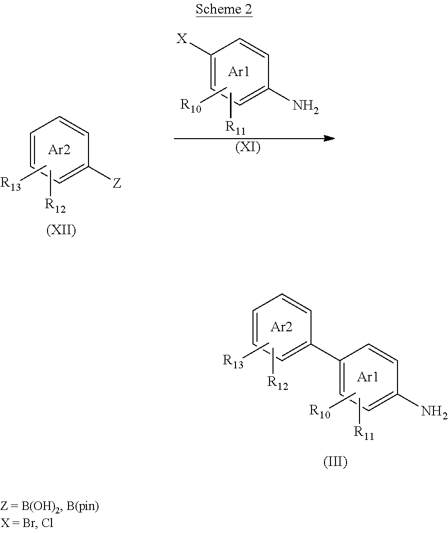

Intermediates of formula (III) wherein Ar1, R₁₀, R₁₁, R₁₂ and R₁₃ are defined above and Ar2 is an unsubstituted or substituted 3-pyridyl ring, may be synthesised by coupling under Suzuki conditions of a boronate of general formula (XII), wherein R₁₂ and R₁₃ are defined above and Z represents a dihydroxyboryl or dialkyloxyboryl group, usually a 4,4,5,5-tetramethyl-1,3,3,2-dioxaborolan-2-yl group, to a substituted pyridine of formula (XI) where X denotes a halide. The couplings according to the Suzuki method are performed, for example, by heating in the presence of a catalyst such as [1,1′-bis(diphenylphosphino)ferrocene]dichloropalladium(II) complex with dichloromethane and an inorganic base such as potassium carbonate in a solvent mixture of dioxane and water.

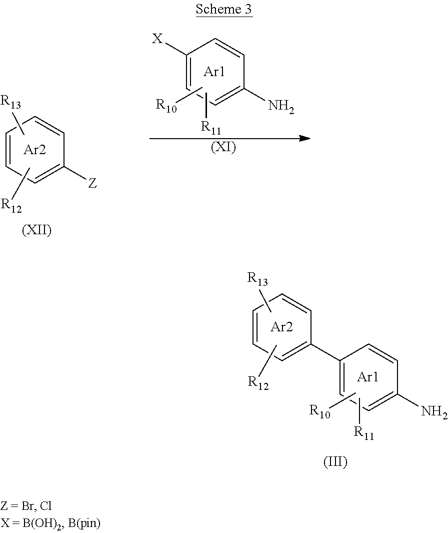

Intermediates of formula (III) wherein Ar1, R₁₀, R₁₁, R₁₂ and R₁₃ are defined above and Ar2 is an unsubstituted or substituted 2,5-pyrazinyl ring, may be synthesised by coupling under Suzuki conditions of an aromatic halide of general formula (XII) and Z represents a halide, to a boronate of general formula (XI) where X denotes a dihydroxyboryl or dialkyloxyboryl group, usually a 4,4,5,5-tetramethyl-1,3,3,2-dioxaborolan-2-yl group. The couplings according to the Suzuki method are performed, for example, by heating in the presence of a catalyst such as tetrakis(triphenylphosphine)palladium or [1,1′-bis(diphenylphosphino)ferrocene]dichloro palladium(II) and an inorganic base such as potassium carbonate in a solvent mixture of dioxane and water.

Benzamide Pyrimidines

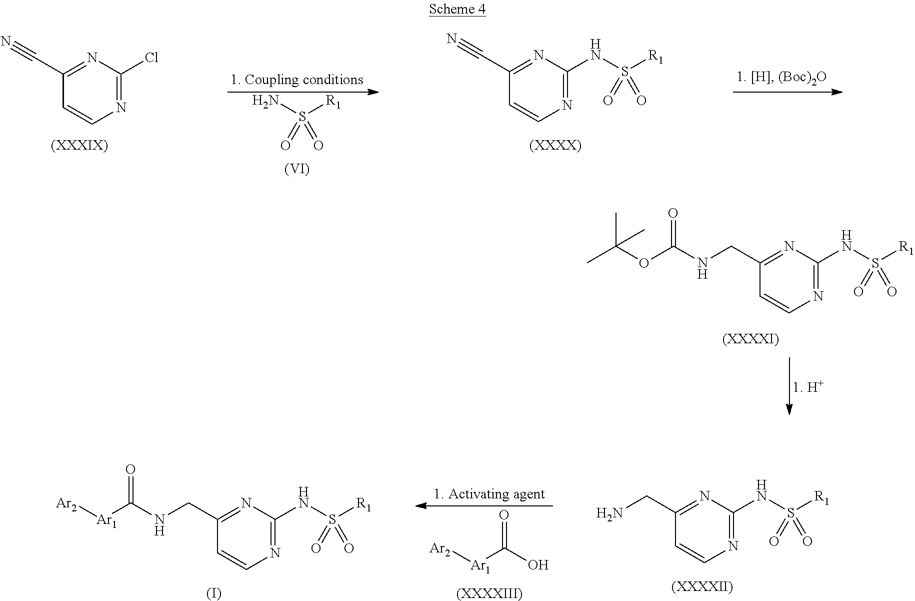

Compounds of general formula (I) may be obtained by a four step process, as shown in Scheme 4. 2-Chloropyrimidine-4-carbonitrile (XXXIX) can be converted to the corresponding sulfonamide (XXXX) using palladium catalysed sulfamination conditions previously reported in Scheme 1a and 1b of WO2019179652. Reduction of the nitrile group using sodium borohydride in the presence of nickel (II) chloride and di-tert-butyl dicarbonate may yield the protected benzylamine derivative of general formula (XXXXI). Deprotection can be carried out by acid hydrolysis using HCl in dioxane to yield benzylamine derivative of general formula (XXXXII). Amide coupling conditions may then be employed to convert the benzylamine derivative (XXXXII) to amides of general formula (I) by employing a coupling reagent together with a biaryl carboxylic acid (XXXXIII) (commercially available or prepared as in Scheme 19 of WO2019179652 or Scheme 6 herein).

Compounds of general formula (I) where A is an amine linker such as —CH₂NH—, where R₁, Ar₁ and Ar₂ are defined above, R₄ is C₁₋₆alkyl and R₅ is H or C₁₋₆alkyl or R₄ and R₅ together with the carbon to which they are attached form a C₃₋₆cycloalkyl or C₃₋₆heterocycloalkyl may be accessed in one step from benzyl amines such as (XXXXII). Reaction of (XXXXII) with aromatic aldehydes (LXXII) in the presence of a hydride source such as sodium triacetoxyborohydride may yield amines of formula (I).

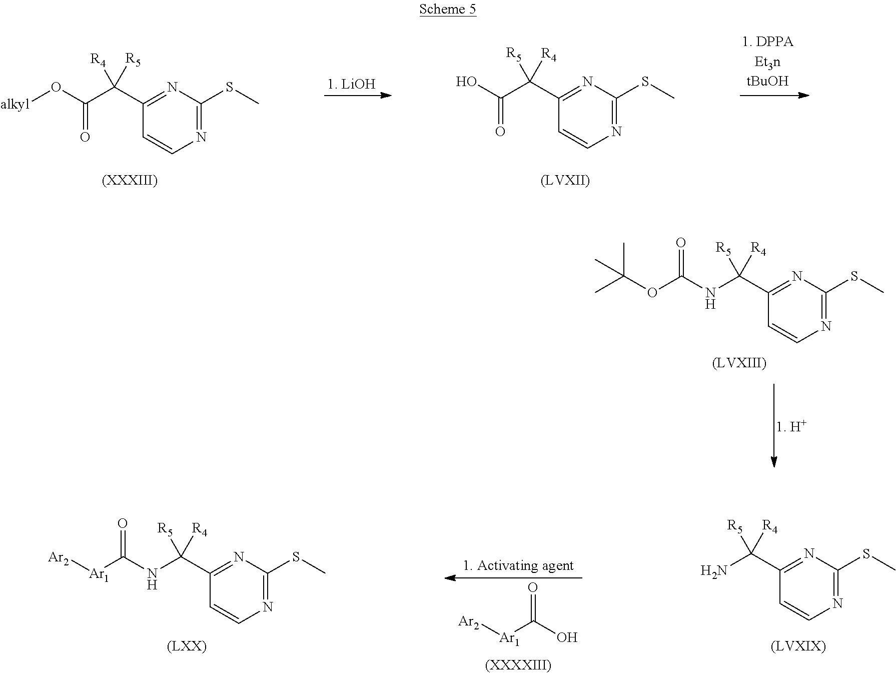

Compounds of general formula (I) where R₁, Ar₁ and Ar₂ are defined above, R₄ is C₁₋₆alkyl and R₅ is H or C₁₋₆alkyl or R₄ and R₅ together with the carbon to which they are attached form a C₃₋₆cycloalkyl or C₃₋₆heterocycloalkyl may be obtained by a six step process, as shown in Scheme 5. Firstly, the carboxylic acid (LVXII) can be obtained by hydrolysis of methyl ester (XXXIII) using an alkali metal base such as lithium hydroxide. Curtius rearrangement can be carried out, for example, using diphenylphosphoryl azide in the presence of propylphosphonic anhydride, triethylamine and tert-butanol to yield carbamates such as (LVXIII). Deprotection can be carried out by acid hydrolysis using HCl in dioxane to yield benzylamine derivative of general formula (LVXIX). Amide coupling conditions may then be employed to convert the benzylamine derivative (LVXIX) to amides of general formula (LXX) by employing a coupling reagent together with a biaryl carboxylic acid (XXXXIII) (commercially available or prepared as in Scheme 19 of WO02019179652). Compound of formula (LXX) can then be progressed to compounds of formula (I) following the oxidation, displacement sequence described in Scheme 9a.

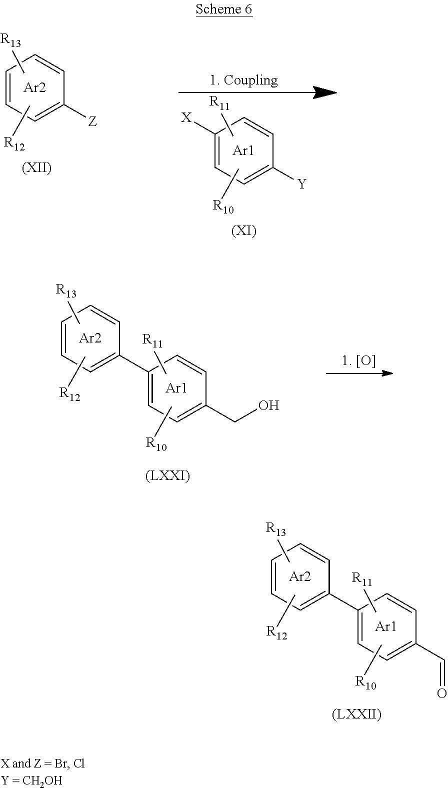

Intermediates of formula (LXXII) where Ar₂ is an unsubstituted or substituted 2-pyrazine ring or 3-pyridyl ring, may be synthesised as shown in Scheme 6, in a one-pot, two step procedure starting with borylation of (XI), where X denotes a halogen such as Br or Cl. followed by coupling under Suzuki conditions with an aromatic halide of general formula (XII), of which R₁₂ and R₁₃ are defined above and Z represents Br or Cl. Initially compounds such as (XI), can be converted to the corresponding boronate using a catalyst such as [1,1′-bis(diphenylphosphino)ferrocene]dichloropalladium(II).CH₂Cl₂ adduct and an inorganic base such as potassium acetate in a solvent such as dioxane. Aromatic halide (XII) may then be added to the reaction mixture along with an aqueous solution of an inorganic base such as caesium carbonate to yield alcohols of formula (LXXI). The aldehydes of general formula (LXXII) are obtained by treatment with an oxidant such as manganese dioxide.

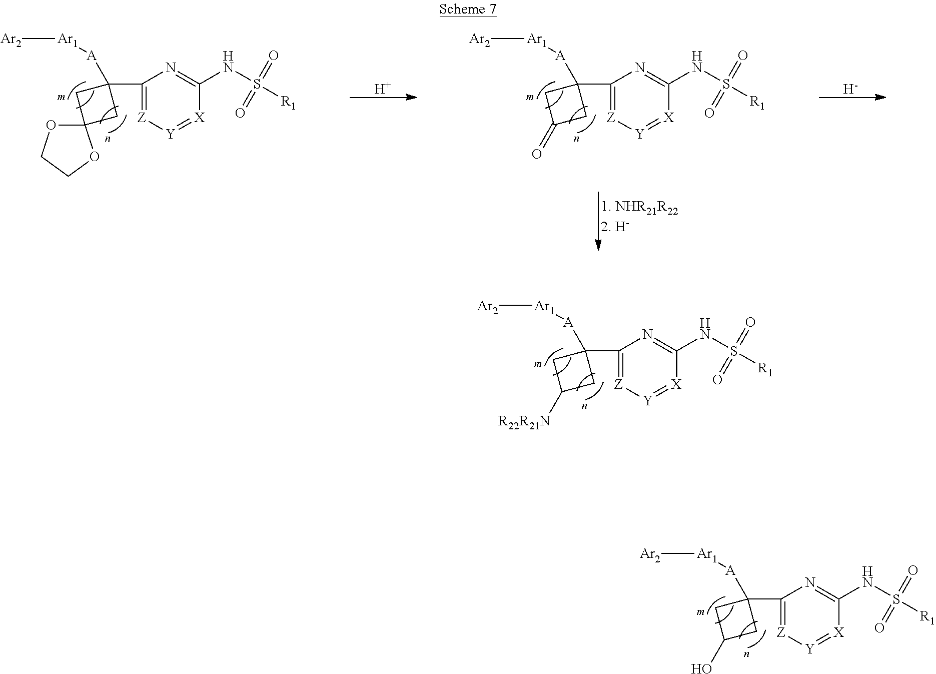

In general compounds of formula (I) where R₄ and R₅ together with the carbon to which they are attached form a 1,4-dioxaspiro[4.5]decane (i.e. m and n are 2) may be treated with a strong acid, such as HCl, to yield cyclic ketones of formula (I). Such ketones may then be treated with a hydride source, such as sodium borohydride, to yield the corresponding exocyclic alcohol or reacted with an amine, such as dimethylamine, followed by sodium triacetoxyborohydride to yield exocydic amines of formula (I).

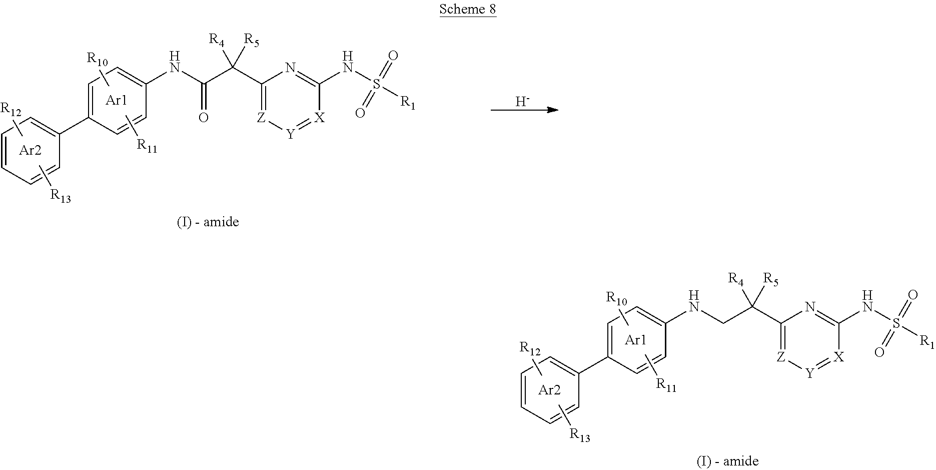

Compounds of formula (I) wherein A is —NR₆CH₂— can be obtained from compounds of formula (I) wherein A is —NR₆C(═O)—, by the reduction of the amide to the amine using a reducing agent such as LiAlH₄ in a solvent such as THF.

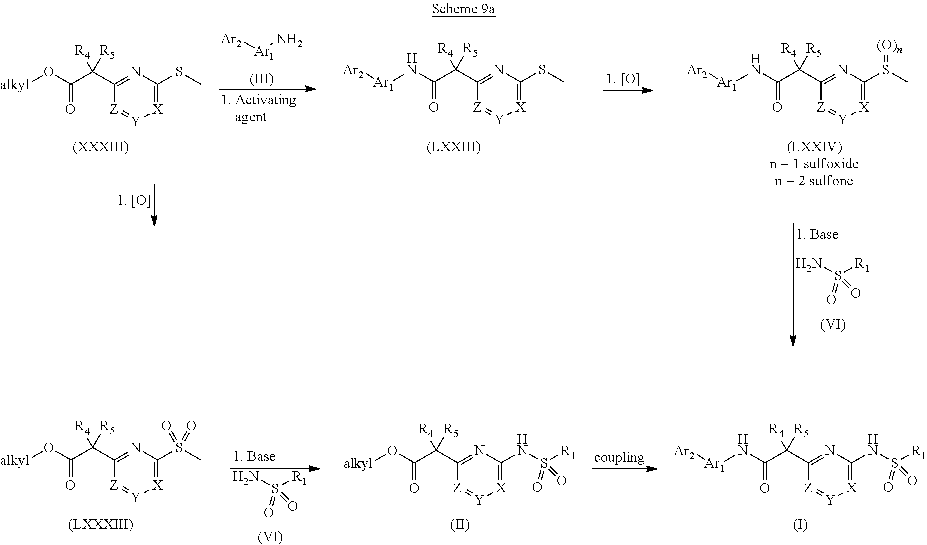

In general and as illustrated in Scheme 9a, compounds of formula (I) wherein R₁, Ar1 and Ar2 are as defined above, alkyl is C₁₋₄alkyl such as methyl or ethyl, e.g. methyl, and for example, R₄ and R₅ together with the carbon atom to which they are attached form a C₃₋₆heterocycloalkyl ring may be prepared starting from a general intermediate of formula (XXXIII). Intermediates such as (LXXIV) may be obtained by subjecting compounds such as (XXXIII) to amide coupling conditions such as those described in Scheme 9 of WO2019179652 using iPrMgCl. Thioethers of the general formula (LXXIII) may be transformed to sulfoxides or sulfones (LXXIV) in the presence of an oxidising agent such as mCPBA. Displacement of the sulfone group with a primary sulphonamide (VI) in the presence of a base such as Cs₂CO₃ and a solvent such as N-methyl pyrrolidone gives compounds of formula (I).

Compounds of formula (I) may also be accessed by oxidation of (XXXIII) to form sulphone (LXXXIII), which can be coupled with (VI), and then (III) using standard conditions disclosed elsewhere herein to give compounds of formula (I).

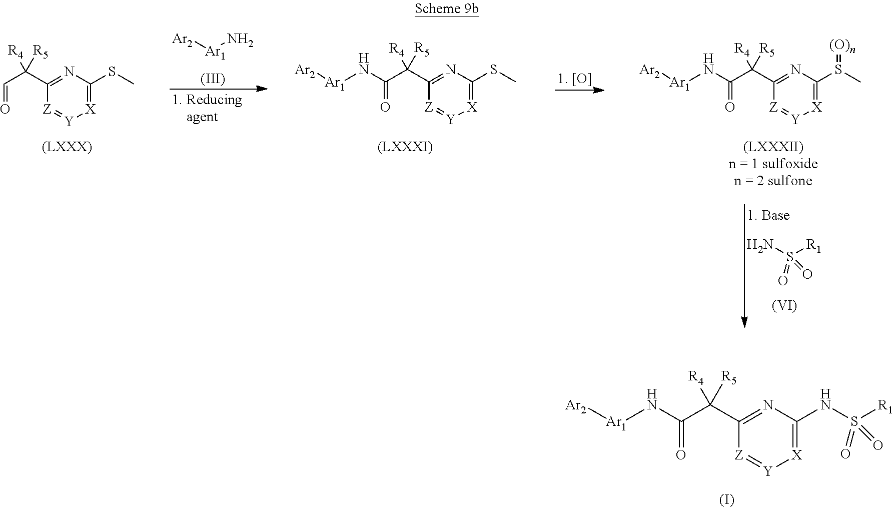

In general and as illustrated in Scheme 9b, compounds of formula (I) wherein R₁, Ar1 and Ar2 are as defined above, alkyl is C₁₋₄alkyl such as methyl or ethyl, e.g. methyl, and for example, R₄ and R₅ together with the carbon atom to which they are attached form a C₃₋₆heterocycloalkyl ring may be prepared starting from a general intermediate of formula (LXXX). Intermediates such as (LXXXI) may be obtained by subjecting compounds such as (LXXX) and (III) to reductive amination conditions such as those described in Scheme 7. Thioethers of the general formula (LXXXI) may be transformed to sulfoxides or sulfones (LXXXII) in the presence of an oxidising agent such as mCPBA. Displacement of the sulfone group with a primary sulphonamide (VI) in the presence of a base such as Cs₂CO₃ and a solvent such as N-methyl pyrrolidone gives compounds of formula (I).

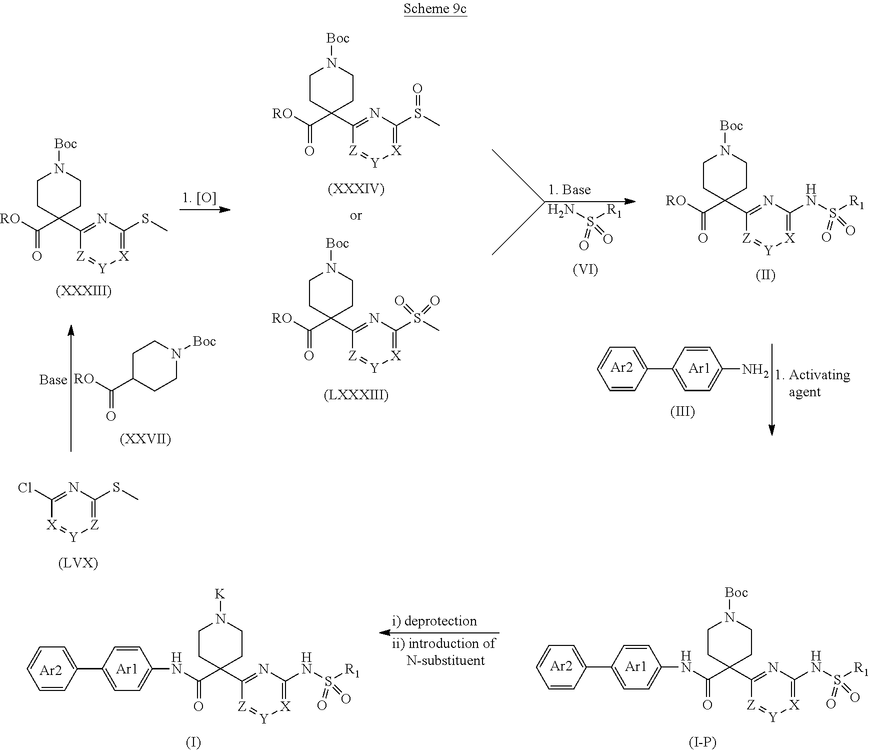

wherein R is H, C₁₋₄alkyl (e.g. methyl and ethyl) or benzyl. In general and as illustrated in Scheme 9c, compounds of formula (I) wherein R₁, X, Y, Z, Ar1 and Ar2 are as defined above and K is C₁₋₄ alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄haloalkyl such as C(O)OtBu, C₁₋₄alkylCN such as CH₂CN, C(O)C₁₋₃alkylOC₁₋₂alkyl such as C(O)CH₂OCH₃, and C₁₋₂alkylC(O)OC₁₋₄alkyl such as CH₂C(O)OCH₂CH₃, may be prepared in five steps from compounds of formula (LVX). An intermediate of formula (XXXVI) may be coupled to a compound of formula (LVX) in the presence of a base such as LiHMDS to give a compound of formula (XXXIII). Thioethers of the general formula (XXXIII) may be transformed to a sulfoxide of formula (XXXIV) or a sulfone of formula (LXXXIII) in the presence of an oxidising agent such as mCPBA. The skilled person will appreciate that formation of the sulfoxide or sulfone can be controlled by varying the number of equivalents of oxidising agent used, the length of reaction and/or the temperature of the reaction. Displacement of the sulfoxide group or the sulfone group with a primary sulfonamide (VI) in the presence of a base such as Cs₂CO₃ and a solvent such as N-methyl pyrrolidone gives compounds of formula (II). Compounds of formula (I) may be obtained by a strong base-mediated amide formation between compounds (II) and (III) at room temperature using bases such as iPrMgCl, UHMDS or KOtBu, to give compounds of formula (I-P), followed by removal of the Boc group using a strong acid such as TFA, and introduction of the N-substituent K. Introduction of K may be performed by reaction of the free NH group with K-LG wherein LG is a leaving group such as halo e.g. chloro or bromo, under standard conditions known to the skilled person, or by any other N-substituent forming conditions known to the skilled person (such as Mitsunobu conditions, reductive amination or N-acylation), to give a compound of formula (I).

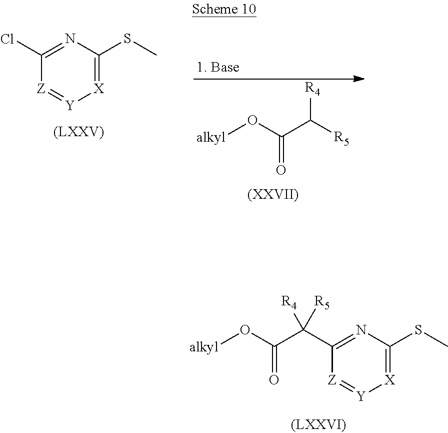

In general and as illustrated in Scheme 10, compounds of formula (I) wherein R₁, Ar1 and Ar2 are as defined above, alkyl is C₁₋₄alkyl such as methyl or ethyl, e.g. methyl, and for example, R₄ and R₅ together with the carbon atom to which they are attached form a C₃₋₆heterocycloalkyl ring may be prepared starting from chloro-pyrimidine (LXXV). Intermediates (XXXVII) are coupled to chloro-pyrimidine (LXXV) in the presence of a base such as LIHMDS to give intermediates (LXXVI). Thioethers of the general formula (LXXVI) may then be transformed to compounds of formula (I) following the route described in Scheme 9a of WO2019179652.

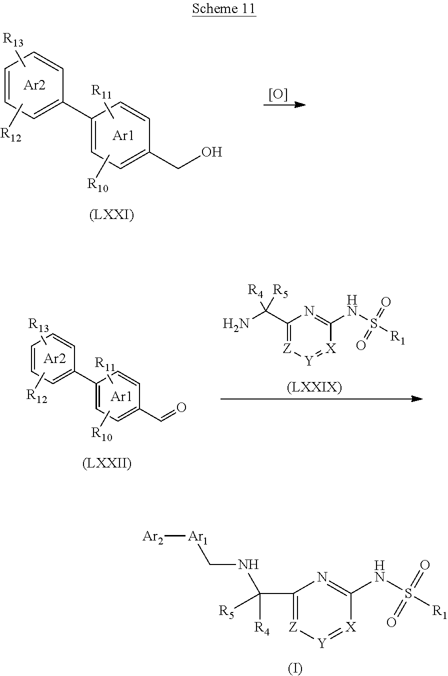

In general and as illustrated in Scheme 11, compounds of formula (I) wherein R1, Ar1 and Ar2 are as defined above, R₄ and R₅ together with the carbon atom to which they are attached form a C₃₋₆heterocycloalkyl ring may be prepared starting from alcohols (LXXI), which as oxidised to aldehydes (LXXII) in the presence of MnO₂ in a non-protic solvent such as DCM. Reductive coupling of amine (LXXIX) and aldehyde (LXXII) in the presence of a hydride source such as sodium triacetoxyborohydride in an aprotic solvent such as DCM in the presence of a proton source such as acetic acid affords compounds of formula (I) following the route described in Scheme 11.

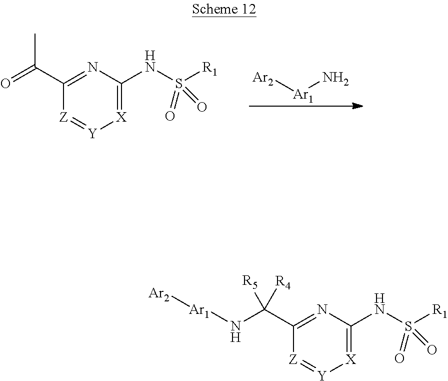

Compounds of formula (I) wherein A is —NH— and R₄ or R₅ is H may be prepared by reductive coupling of the appropriate amine and aldehyde in the presence of a hydride source such as sodium triacetoxyborohydride.

Compounds of Formula (I-b)

Compounds of formula (I-b) may be synthesised by general and specific methods disclosed in WO2019/106156 and those disclosed below. For the avoidance of doubt, reference to variables in each of the schemes in this section encompasses variables specific to compounds of formula (I-b). For example, R₁ includes R_{1b}. Furthermore, reference to intermediates in each of the schemes in this section encompasses corresponding intermediates for compounds of formula (I-b). For example, reference to compounds of formula (II) encompasses compounds of formula (II-b).

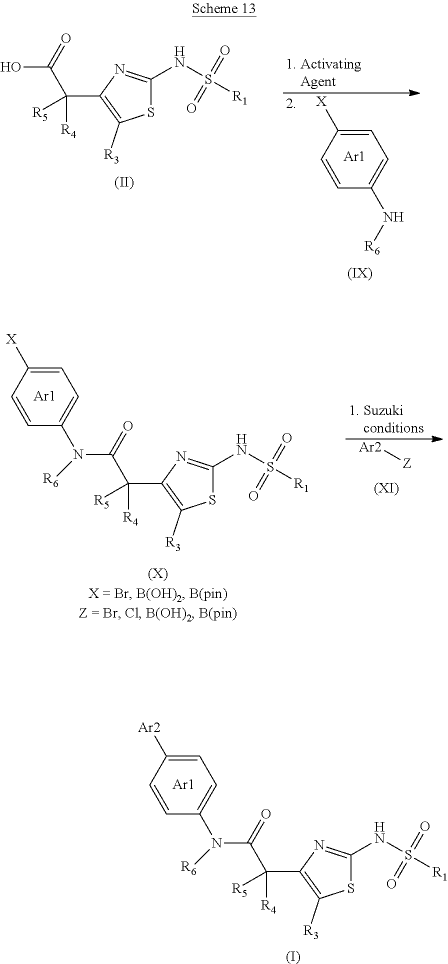

Compounds of formula (I) may be obtained by a general process whereby a carboxylic acid precursor (II), or a suitably protected derivative thereof, is reacted with an activating agent, to generate a reactive, electrophilic carboxylic acid derivative, followed by subsequent reaction with an amine of formula (IX). Intermediates of formula (X) are then converted to a compound of the invention of general formula (I) by coupling under Suzuki conditions with an aromatic halide or boronate of general formula (XI), of which X is defined above and represents usually a bromide, a dihydroxyboryl or dialkyloxyboryl group, usually a 4,4,5,5-tetramethyl-1,3,3,2-dioxaborolan-2-yl group. The couplings according to the Suzuki method are performed, for example, by heating in the presence of a catalyst such as bis(diphenylphosphino)ferrocene]dichloropalladium(II) and an inorganic base such as potassium carbonate in a solvent mixture of dioxane and water. It will be understood by persons skilled in the art that many catalysts and conditions can be employed for such couplings.

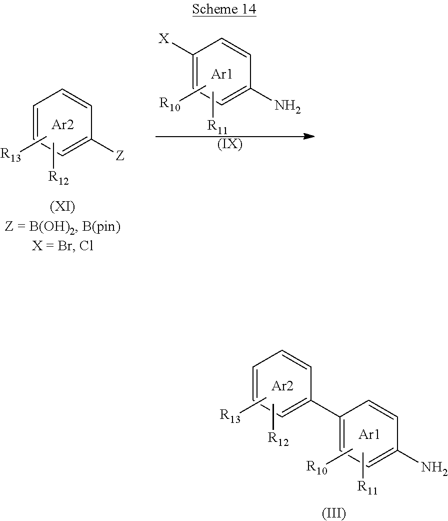

Intermediates of formula (III) where Ar2 is an unsubstituted or substituted 3-pyridyl ring, may be synthesised by coupling under Suzuki conditions of a boronate of general formula (XI), of which R¹² and R¹³ are defined above and Z represents a dihydroxyboryl or dialkyloxyboryl group, usually a 4,4,5,5-tetramethyl-1,3,3,2-dioxaborolan-2-yl group, to a substituted pyridine of formula (IX) of which R¹⁰ and R¹¹ are defined above and where X denotes a halide. The couplings according to the Suzuki method are performed, for example, by heating in the presence of a catalyst such as [1,1′-bis(diphenylphosphino)ferrocene]dichloropalladium(II) complex with dichloromethane and an inorganic base such as potassium carbonate in a solvent mixture of 1,4-dioxane and water.

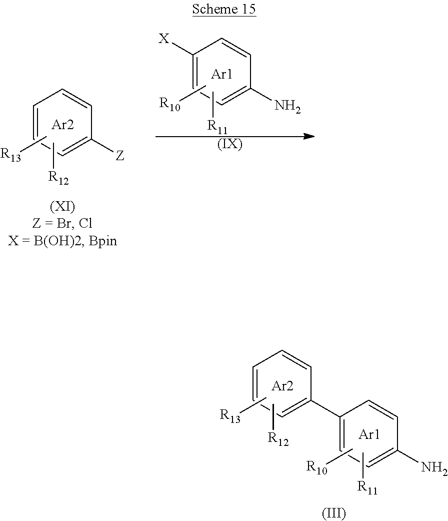

Intermediates of formula (III) where Ar2 is an unsubstituted or substituted 2-pyrazine ring, may be synthesised by coupling under Suzuki conditions of an aromatic halide of general formula (XI), of which R¹² and R¹³ are defined above and Z represents a halide, to a boronate of general formula (IX) of which R¹⁰ and R¹¹ are defined above and where X denotes a dihydroxyboryl or dialkyloxyboryl group, usually a 4,4,5,5-tetramethyl-1,3,3,2-dioxaborolan-2-yl group. The couplings according to the Suzuki method are performed, for example, by heating in the presence of a catalyst such as tetrakis(triphenylphosphine)palladium or [1,1′-bis(diphenylphosphino) ferrocene]dichloropalladium(II) and an inorganic base such as potassium carbonate in a solvent mixture of dioxane and water.

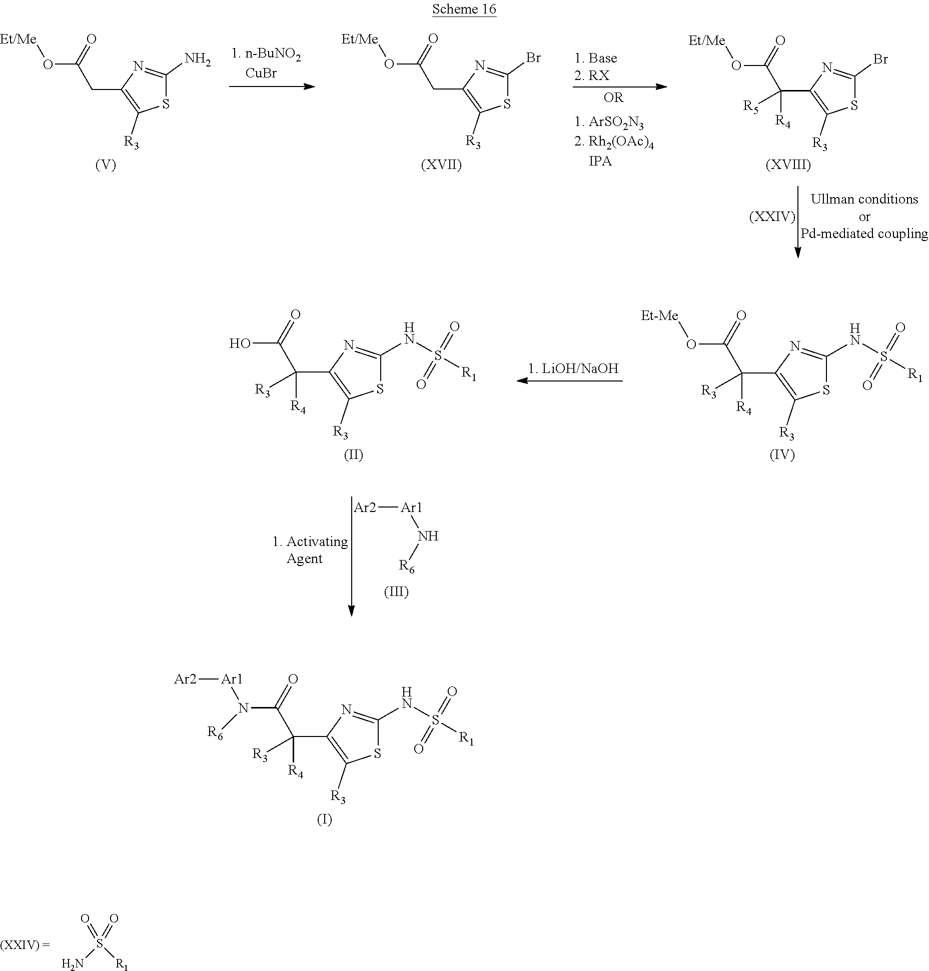

Compound of general formula (XVII) may be prepared by conversion of a suitable 2-(2-aminothiazol-4-yl)acetate derivative, such as a methyl or ethyl derivative, by a Sandmeyer type reaction using an organic nitrite, such as n-butylnitrite, in the presence of a halide source, such as Cu(I)Br in acetonitrille. Such reactions can be undertaken at temperatures of RT to 60° C.

Introduction of R₄/R₅can be undertaken by two alternative methods at this stage. Firstly, alkylation of compounds of general formula (XVII) can be undertaken by addition of a suitable base, for example, LiHMDS, together with an alkylating agent, such as iodomethane which results in dialkylation alpha to the ester moiety to yield compounds of formula (XVIII), where R_{4b}=R_{5b}=Me. Secondly, diazotisation of compounds of general formula (XVII) with the use of an diazo transfer reagent, such as 4-acetamidobenzenesulfonyl azide, under basic conditions, followed by treatment with rhodium and subsequent insertion of the corresponding nucleophile, such as isopropyl alcohol, gives intermediates of general formula (XVIII) where R_{4b}=Oisopropoxy and R_{5b}=H.

Introduction of the sulfonamide group in the preparation of compounds of formula (IV) may be achieved by an Ullmann coupling reaction i.e. Cu mediated coupling conditions using amines of formula (XXIV) and a copper catalyst, such as Cu(I)I, in the presence of an inorganic base, potassium carbonate, and a diamine ligand in dioxane. Such reactions are typically carried out at elevated temperatures such as 80° C. Alternatively, conversion of compounds of formula (XVIII) to (IV) can be achieved via a palladium mediated coupling, for example using a catalyst such as [t-BuXPhos Pd(allyl)]OTf and substituted sulfonamide nucleophile (XXIV), in the presence of an inorganic base, for example potassium carbonate to form compounds of formula (IV). Palladium meditated coupling conditions are particularly useful when R₄ and R₅ together with the carbon atom to which they are attached form a 5- or 6-membered heterocycloalkyl, such as a tetrahydropyranyl. The alkyl esters of formula (IV) may be conveniently converted to compounds of formula (I) according to synthetic steps reported in Scheme 13.

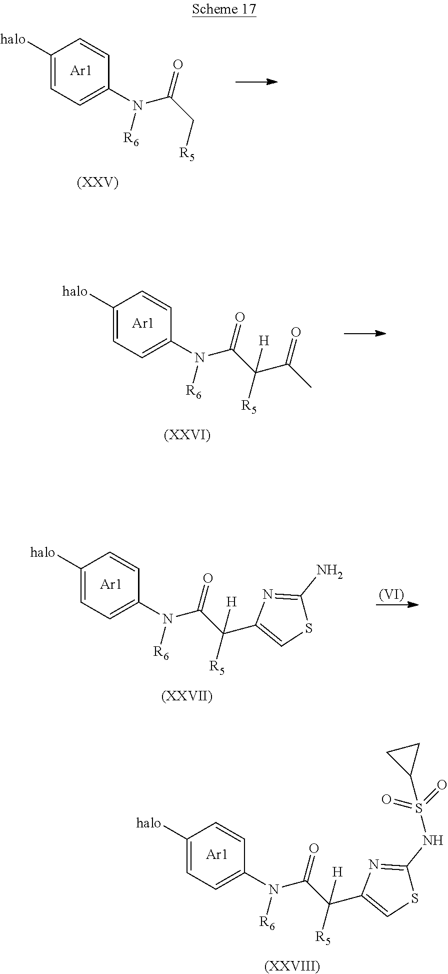

wherein halo is, for example, bromo. Compounds of formula (XXVII), for example, when R_{4b} is H and R_{5b} and R₆ are a C₂₋₃alkylene chain forming a 5- or 6-membered ring, can be accessed in three steps from compounds of formula (XXV). Acylation of compounds of formula (XXV) in the presence of a strong base such as LDA followed by a quench with an ⁻OAc source such as EtOAc provides compounds of formula (XXVI). Compounds of formula (XXVII) can be made from (XXVI) following addition of bromine and quench with a suitable thiourea, before a coupling with sulfonyl chlorides of formula (VI) to give compounds of formula (XXVIII). Compounds of formula (I) may be accessed using conditions set out in Scheme 14.

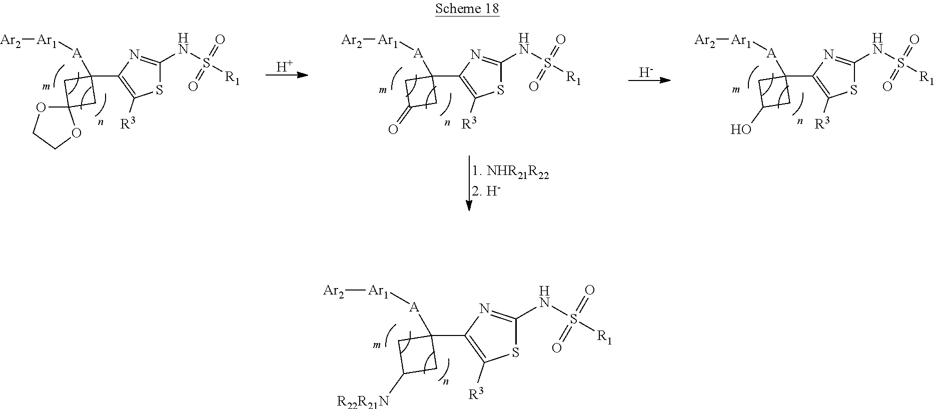

In general compounds of formula (I) where R₄ and R₅ together with the carbon to which they are attached form a 1,4-dioxaspiro[4.5]decane (i.e. m is 2 and n is 2) may be treated with a strong acid such as HCl to yield cyclic ketones of formula (I). Such ketones may then be treated with a hydride source such as sodium borohydride to yield the corresponding exocyclic alcohol or reacted with an amine such as dimethylamine followed by sodium triacetoxyborohydride to yield exocyclic amines of formula (I).

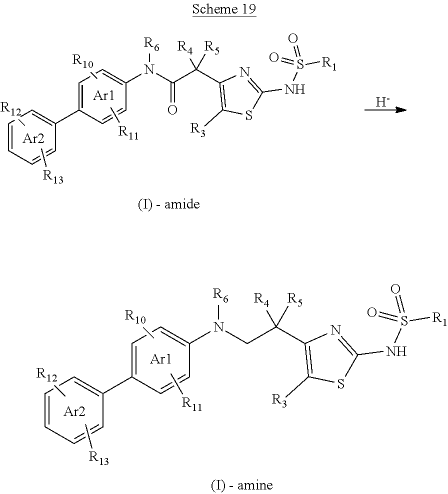

Compounds of formula (I) wherein A is —NR₆CH₂— can be obtained from compounds of formula (I) wherein A is —NR₆C(═O)—, by the reduction of the amide to the amine using a reducing agent such as LiAlH₄ in a solvent such as THF.

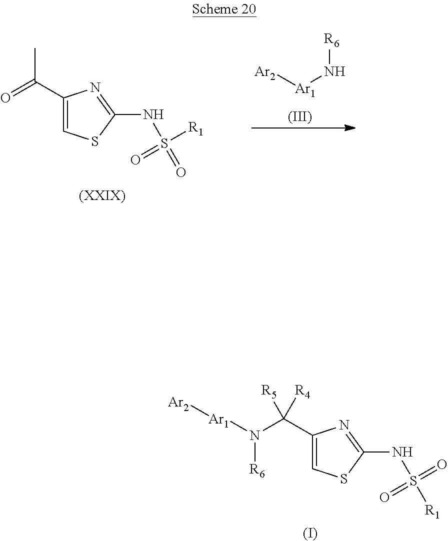

Compounds of formula (I) wherein A is —NR₆— and R₅ or R₄ are H can be obtained in a single step by a reductive amination between ketones of formula (XXIX) and amines of formula (III) in the presence of a hydride source such as sodium triacetoxyborohydride.

Compounds of Formula (I-c)

Compounds of formula (I-c) may be synthesised by general and specific methods disclosed in WO02019/106148 and those disclosed below. For the avoidance of doubt, reference to variables in each of the schemes in this section encompasses variables specific to compounds of formula (I-c). For example, R₁ includes R_{1c}. Furthermore, reference to intermediates in each of the schemes in this section encompasses corresponding intermediates for compounds of formula (I-c). For example, reference to compounds of formula (II) encompasses compounds of formula (II-c).

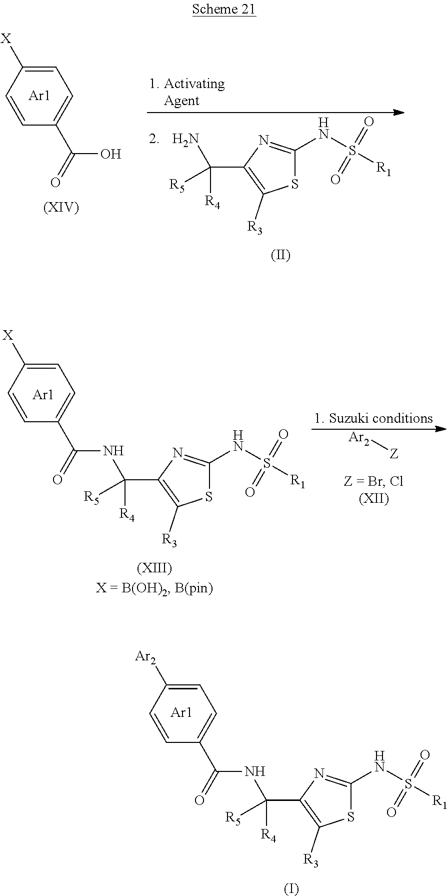

Compounds of formula (XIII) may be obtained by a general process as shown in Scheme 21 whereby a carboxylic acid precursor (XIV) is reacted with an activating agent such as HATU, T3P or Ghosez's reagent, to generate a reactive, electrophilic carboxylic acid derivative, followed by subsequent reaction with an amine of formula (II). Intermediates of formula (XIII) are then converted to a compound of general formula (I) by coupling under Suzuki conditions with an aromatic halide of general formula (XII), of which X is defined in Scheme 21 and represents a dihydroxyboryl or dialkyloxyboryl group, such as a 4,4,5,5-tetramethyl-1,3,3,2-dioxaborolan-2-yl group. The couplings according to the Suzuki method are performed, for example, by heating in the presence of a catalyst such as bis(diphenylphosphino)ferrocene]dichloropalladium(II).CH₂Cl₂ adduct and an inorganic base such as potassium carbonate in a solvent mixture of dioxane and water under an inert atmosphere such as a nitrogen atmosphere. It will be understood by persons skilled in the art that many catalysts and conditions can be employed for such couplings.

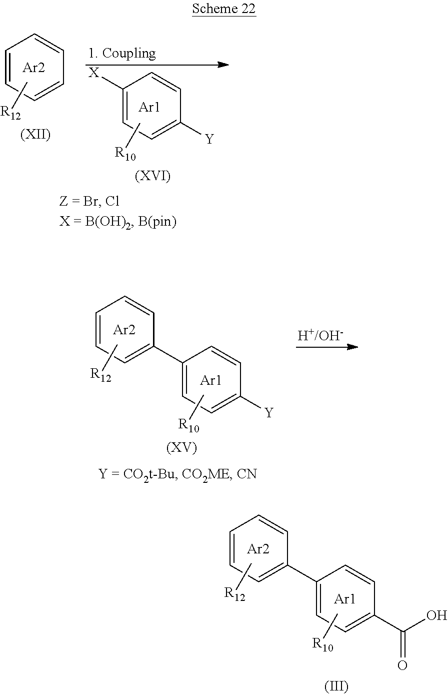

Intermediates of formula (III) where Ar₂ is an unsubstituted or substituted 2-pyrazine ring or 3-pyridyl ring, may be synthesised as shown in Scheme 22 by coupling under Suzuki conditions of an aromatic halide of general formula (XII), of which R₁₀ and R₁₂ are defined above and Z represents a halide such as Br or Cl, to a boronate of general formula (XVI) where X denotes a dihydroxyboryl or dialkyloxyboryl group, such as a 4,4,5,5-tetramethyl-1,3,3,2-dioxaborolan-2-yl group. The couplings according to the Suzuki method are performed, for example, by heating in the presence of a catalyst such as [1,1′-bis(diphenylphosphino)ferrocene]dichloropalladium(II).CH₂Cl₂ adduct and an inorganic base such as cesium carbonate in a solvent mixture of dioxane and water under an inert atmosphere such as a nitrogen atmosphere. The carboxylic acids of general formula (III) are obtained by either deprotection of the t-butyl ester using a strong acid, such as TFA in a solvent of CH₂Cl₂, hydrolysis of the methyl ester using an alkali metal hydroxide such as NaOH in a solvent mixture such as THF/MeOH or hydrolysis of the nitrile using a strong acid such as concentrated HCl. Compounds of formula (III-A) may also be made using this method.

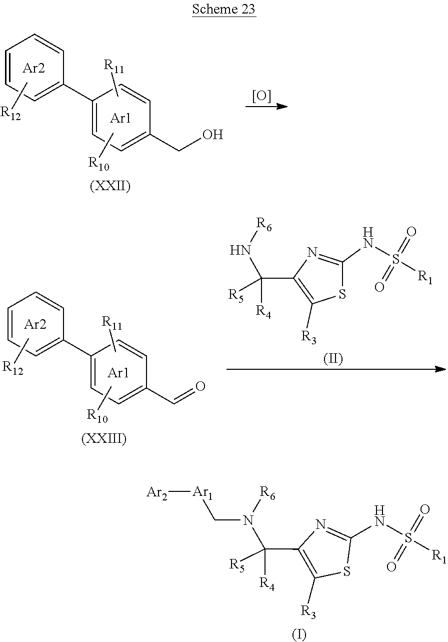

Compounds of formula (I) wherein Aₐ is —CH₂NR₆— can be accessed in two steps from compounds of formula (XXII). Oxidation of the alcohol (XXII) under standard oxidation conditions such as MnO₂ gives aldehyde (XXIII). A reductive amination using a hydride source such as sodium tiacetoxyborohydride between aldehyde (XXIII) and amine (II) gives compounds of formula (I).

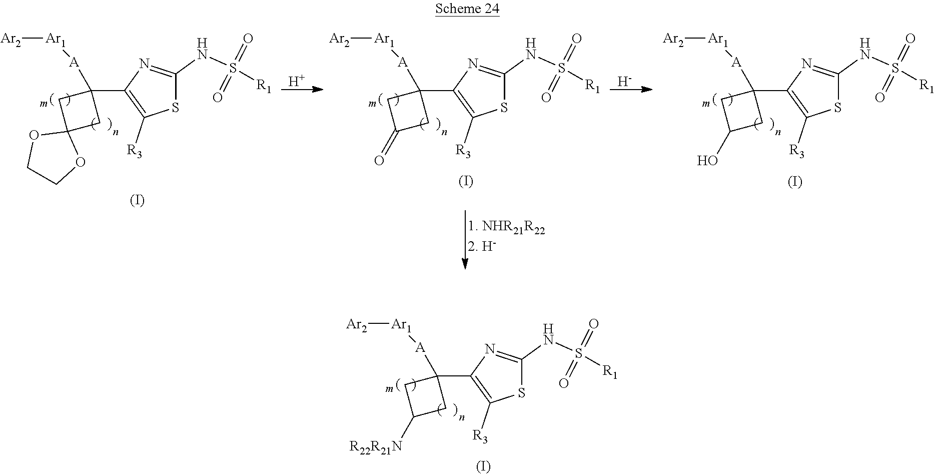

In general compounds of the formula (I) where R₄ₐ and R₅ₐ together with the carbon to the which they are attached form a C₃₋₆cycloalkyl wherein one of the carbons of the C₃₋₆cycloalkyl is a spiro centre such that spirocyclic ring system is formed by the C₃₋₆cycloalkyl ring and a C₃₋₆heterocycloalkyl ring, such as a 1,4-dioxaspiro[4.5]decane moiety (m is 2 and n is 2), may be treated with a strong acid such as HCl to yield cyclic ketones of formula (I). Such ketones may then be treated with a hydride source such as sodium borohydride to yield the corresponding exocyclic alcohol or reacted with an amine such as dimethylamine followed by sodium triacetoxyborohydride to yield exocyclic amines of formula (I).

### Intermediates of the Invention

Compounds of Formula (I-a)

The present invention also relates to novel intermediates in the synthesis of compounds of formula (I-a) such as compounds of formula (II-a) to (LVIX-a) such as compounds of formula (II-a) to (XXV-a), such as compounds of formula (II-a)-(XX-a). Particular intermediates of interest are those of the following general formulae, wherein the variable groups and associated preferences are as defined previously for compounds of formula (I-a):
a compound of formula (II-a):

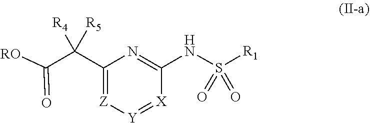

wherein R is H, C₁₋₆alkyl (e.g. methyl and ethyl) or benzyl;
a compound of formula (XX-a):

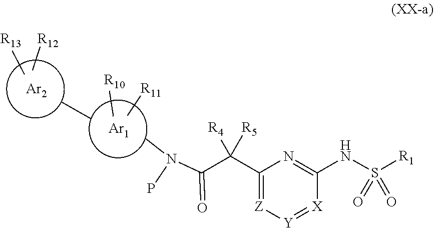

wherein P is a nitrogen protecting group such as para-methoxybenzyl;
a compound of formula (XXIV-a):

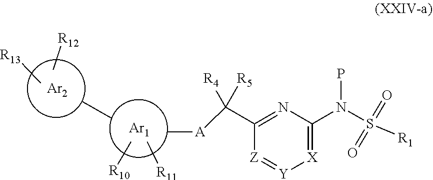

wherein P is a nitrogen protecting group such as para-methoxybenzyl;
a compound of formula (XXXI-a):

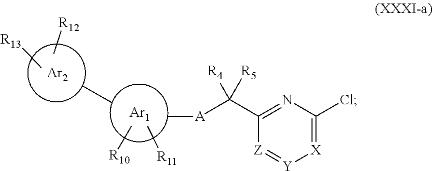

a compound of formula (XXXXII-a):

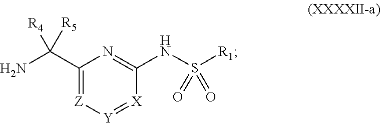

a compound of formula (LI-a):

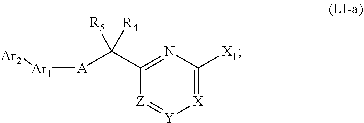

wherein X₁ is Cl or Br.
a compound of formula (LVIII-a):

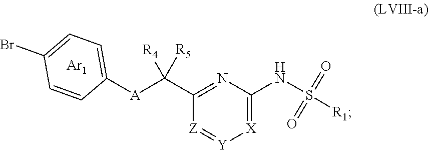

a compound of formula (LXXIII-a):

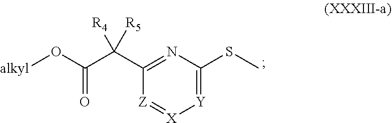

wherein alkyl is C₁₋₄alkyl such as methyl or ethyl, e.g. methyl;
a compound of formula (LXXIII-a)

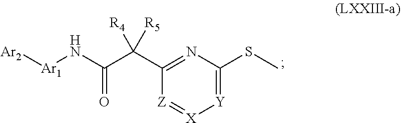

a compound of formula (LXXIV-a):

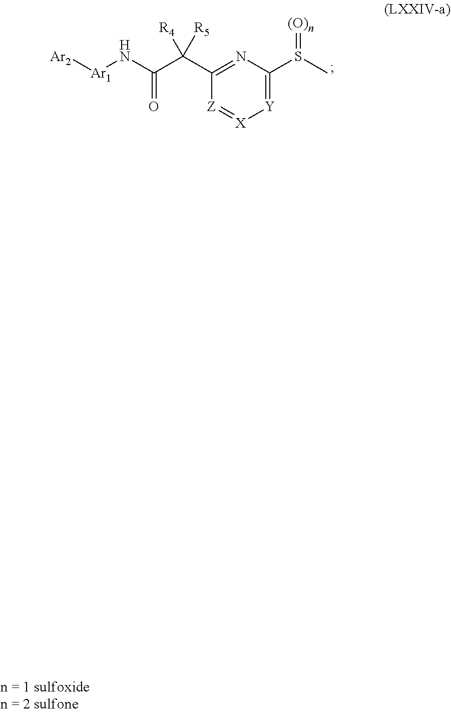

a compound of formula (LXXXIII-a):

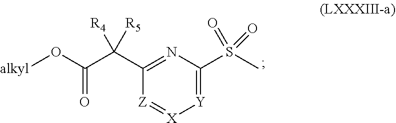

a compound of formula (XXXIV-a):

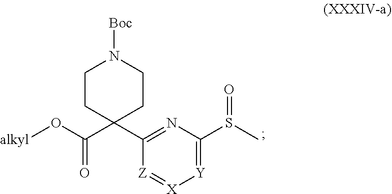

a compound of formula (LXXI-a):

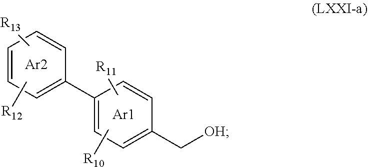

a compound of formula (LXXII-a):

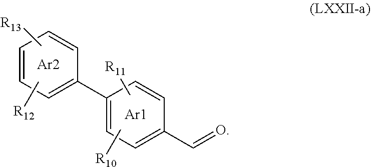

Suitably, at least one of R₁₀, R₁₁ and R₁₂ is other than H.

There is also provided protected derivatives of a compound of formula (I). There is also provided protected derivatives of any one of the above intermediates, such as (II-a), (XX-a), (XXIV-a), (XXXI-a), (XXXXII-a), (LI-a), (LVIII-a), (XXXIII-a), (LXXIII-a), (LXXIV-a) and (LXXXIII-a). For example, if the compound of formula (I) comprises a heterocyclyl group comprising a nitrogen atom, for example, when R_{4ba} and R_{5ba} join to form a piperidinyl ring, the nitrogen atom may be protected with a suitable nitrogen protecting group such as Boc. Also provided are compounds of formula (I) or any one of the above mentioned intermediates in which the protecting group, such as the Boc group has been removed.

Compounds of formula (I) may be considered intermediates for further compounds of formula (I), as described in the Examples below.

Included as an aspect of the invention are all novel intermediates described in the examples, including:
Intermediates INTC186 to INTC218; and
Intermediates INTC232 to INTC247.

Included as an aspect of the invention are salts such as pharmaceutically acceptable salts of any one of the intermediates disclosed herein, such as any one of compounds of formulae (II-a)-(LXXVII-a).

Compounds of Formula (I-b)

The present invention also relates to novel intermediates in the synthesis of compounds of formula (I-b) such as compounds of formula (II-b), (IV-b), (V-b), (VI-b), (VII-b), (X-b), (XII-b), (XVIII-b), (XIX-b), (XX-b), (XXI-b), (XXII-b) and (XXIII-b). Particular intermediates of interest are those of the following general formulae, wherein the variable groups and associated preferences are as defined previously for compounds of formula (I-b):
Compounds of formula (II-b)

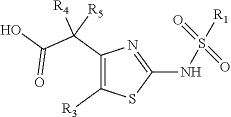

wherein R₁, R₃, R₄ and R₅ are as defined herein;
Compounds of formula (IV-b)

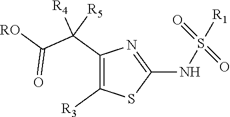

wherein R is C₁₋₆alkyl (e.g. methyl, ethyl) or benzyl.

Suitably, the intermediate is not:

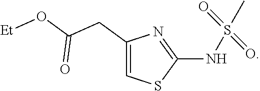 [SMILES: CCOC(=O)Cc1csc(NS(C)(=O)=O)n1]

Suitably, when the compound is a compound of formula (IV-b), R_{4b} and R_{5b} cannot both be H when R_{1b} is CH₃.

Included as an aspect of the invention are salts such as pharmaceutically acceptable salts of any one of the intermediates disclosed herein, such as any one of compounds of formulae (II-b), (IV-b), (V-b), (VI-b), (VII-b), (X-b), (XII-b), (XVIII-b), (XIX-b), (XX-b), (XXI-b), (XXII-b) and (XXIII-b).

Other intermediates of interest include those disclosed in WO2019/106156 such as INTA92, INTA93, INTA94, INTA95, INTA96, INTA97, INTA98, INTA103, INTA104, INTA105, INTA107, INTA3, INTA47, INTA48, INTA72, INTA106, INTB37, INTB38, INTB43.

Compounds of Formula (I-c)

The present invention also relates to novel intermediates in the synthesis of compounds of formula (I-c) such as compounds of formula (II-c), (IV-c), (V-c), (VI-c), (VIII-c), (IX-c), (X-c), (XI-c), (XIII-c), (XVIII-c), (XIX-c), (XX-c) and (XXI-c). Particular intermediates of interest are those of the following general formulae, wherein the variable groups and associated preferences are as defined previously for compounds of formula (I-c):
a compound of formula (II-c):

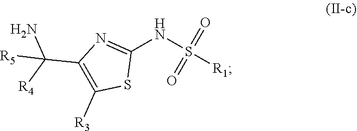

a compound of formula (VIII-c):

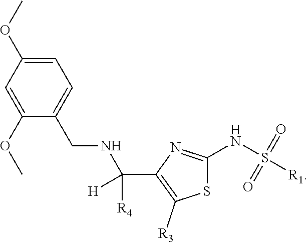

Included as an aspect of the invention are salts such as pharmaceutically acceptable salts of any one of the intermediates disclosed herein, such as any one of compounds of formulae (II-c), (IV-c), (V-c), (VI-c), (VIII-c), (IX-c), (X-c), (XI-c), (XIII-c), (XVIII-c), (XIX-c), (XX-c) and (XXI-c).

Therapeutic Methods

In the following section, reference to compounds of formula (I) encompasses compounds of formula (I-a), (I-b) and (I-c).

Compounds of formula (I) of the present invention have utility as inhibitors of CTPS1.

Therefore, the invention also provides a compound of formula (I), or a pharmaceutically acceptable salt and/or solvate (e.g. salt) and/or derivative thereof, for use as a medicament, in particular in the treatment or prophylaxis of a disease or disorder wherein an inhibitor of CTPS1 is beneficial, for example those diseases and disorders mentioned herein below.

The invention provides a method for the treatment or prophylaxis of a disease or disorder wherein an inhibitor of CTPS1 is beneficial, for example those diseases and disorders mentioned herein below, which comprises administering to a subject in need thereof an effective amount of a compound of formula (I) or a pharmaceutically acceptable salt and/or solvate (e.g. salt) and/or derivative thereof.

The invention also provides the use of a compound of formula (I), or a pharmaceutically acceptable salt and/or solvate thereof (e.g. salt) and/or derivative, in the manufacture of a medicament for the treatment or prophylaxis of a disease or disorder wherein an inhibitor of CTPS1 is beneficial, for example those diseases and disorders mentioned herein below.

More suitably, the disease or disorder wherein an inhibitor of CTPS1 is beneficial is a disease or disorder wherein a reduction in T-cell and/or B-cell proliferation would be beneficial.

The invention also provides a compound of formula (I), or a pharmaceutically acceptable salt and/or solvate (e.g. salt) and/or derivative thereof, for use in the inhibition of CTPS1 in a subject.

The invention provides a method for the inhibition of CTPS1 in a subject, which comprises administering to the subject an effective amount of a compound of formula (I) or a pharmaceutically acceptable salt and/or solvate (e.g. salt) and/or derivative thereof.

The invention also provides the use of a compound of formula (I), or a pharmaceutically acceptable salt and/or solvate thereof (e.g. salt) and/or derivative, in the manufacture of a medicament for the inhibition of CTPS1 in a subject

The invention also provides a compound of formula (I), or a pharmaceutically acceptable salt and/or solvate (e.g. salt) and/or derivative thereof, for use in the reduction of T-cell and/or B-cell proliferation in a subject

The invention provides a method for the reduction of T-cell and/or B-cell proliferation in a subject, which comprises administering to the subject an effective amount of a compound of formula (I) or a pharmaceutically acceptable salt and/or solvate (e.g. salt) and/or derivative thereof.

The invention also provides the use of a compound of formula (I), or a pharmaceutically acceptable salt and/or solvate thereof (e.g. salt) and/or derivative, in the manufacture of a medicament for the reduction of T-cell and/or B-cell proliferation in a subject More suitably, the disease or disorder wherein an inhibitor of CTPS1 is beneficial is a disease or disorder wherein a reduction in T-cell and/or B-cell proliferation would be beneficial.

The term ‘treatment’ or ‘treating’ as used herein includes the control, mitigation, reduction, or modulation of the disease state or its symptoms.

The term ‘prophylaxis’ or ‘preventing’ is used herein to mean preventing symptoms of a disease or disorder in a subject or preventing recurrence of symptoms of a disease or disorder in an afflicted subject and is not limited to complete prevention of an affliction.

Suitably, the disease or disorder is selected from rejection of transplanted cells and tissues, Graft-related diseases or disorders, allergies and autoimmune diseases.

In one embodiment the disease or disorder is the rejection of transplanted cells and tissues. The subject may have been transplanted with a graft selected from the group consisting of heart, kidney, lung, liver, pancreas, pancreatic islets, brain tissue, stomach, large intestine, small intestine, cornea, skin, trachea, bone, bone marrow (or any other source of hematopoietic precursor cells and stem cells including hematopoietic cells mobilized from bone marrow into peripheral blood or umbilical cord blood cells), muscle, or bladder. The compounds of the invention may be of use in preventing or suppressing an immune response associated with rejection of a donor tissue, cell, graft or organ transplant in a subject In a further embodiment the disease or disorder is a Graft-related disease or disorder. Graft-related diseases or disorders include graft versus host disease (GVHD), such as GVHD associated with bone marrow transplantation, and immune disorders resulting from or associated with rejection of organ, tissue, or cell graft transplantation (e.g., tissue or cell allografts or xenografts), including, e.g., grafts of skin, muscle, neurons, islets, organs, parenchymal cells of the liver, etc, and Host-Versus-Graft-Disease (HVGD). The compounds of the invention may be of use in preventing or suppressing acute rejection of such transplant in the recipient and/or for long-term maintenance therapy to prevent rejection of such transplant in the recipient (e.g., inhibiting rejection of insulin-producing islet cell transplant from a donor in the subject recipient suffering from diabetes). Thus the compounds of the invention have utility in preventing Host-Versus-Graft-Disease (HVGD) and Graft-Versus-Host-Disease (GVHD).

A CTPS1 inhibitor may be administered to the subject before, after transplantation and/or during transplantation. In some embodiments, the CTPS1 inhibitor may be administered to the subject on a periodic basis before and/or after transplantation.

In another embodiment, the disease or disorder is an allergy.

In additional embodiments the immune related disease or disorder is an autoimmune disease. As used herein, an “autoimmune disease” is a disease or disorder directed at a subject's own tissues. Examples of autoimmune diseases include, but are not limited to Addison's Disease, Adult-onset Still's disease, Alopecia Areata, Alzheimer's disease, Anti-neutrophil Cytoplasmic Antibodies (ANCA)-Associated Vasculitis, Ankylosing Spondylitis, Anti-phospholipid Syndrome (Hughes' Syndrome), A plastic Anemia, Arthritis, Asthma, Atherosclerosis, Atherosclerotic plaque, Atopic Dermatitis, Autoimmune Hemolytic Anemia, Autoimmune Hepatitis, Autoimmune Hypophysitis (Lymphocytic Hypophysitis), Autoimmune Inner Ear Disease, Autoimmune Lymphoproliferative Syndrome, Autoimmune Myocarditis, Autoimmune Neutropenia, Autoimmune Oophoritis, Autoimmune Orchitis, Auto-Inflammatory Diseases requiring an immunosuppressive treatment, Azoospermia, Bechet's Disease, Berger's Disease, Bullous Pemphigoid, Cardiomyopathy, Cardiovascular disease, Celiac disease including Refractory Celiac Disease (type I and type II), Chronic Fatigue Immune Dysfunction Syndrome (CFIDS), Chronic Idiopathic Polyneuritis, Chronic Inflammatory Demyelinating Polyneuropathy (CIPD), Chronic Relapsing Polyneuropathy (Guillain-Barré syndrome), Churg-Strauss Syndrome (CSS), Cicatricial Pemphigoid, Cold Agglutinin Disease (CAD), chronic obstructive pulmonary disease (COPD), CREST Syndrome, Cryoglobulin Syndromes, Cutaneous Lupus, Dermatitis Herpetiformis, Dermatomyositis, Eczema, Epidermolysis Bullosa Acquisita, Essential Mixed Cryoglobulinemia, Evan's Syndrome, Exophthalmos, Fibromyalgia, Goodpasture's Syndrome, Grave's disease, Hemophagocytic Lymphohistiocytosis (HLH) (including Type 1 Hemophagocytic Lymphohistiocytosis), Histiocytosis/Histiocytic Disorders, Hashimoto's Thyroiditis, Idiopathic Pulmonary Fibrosis, Idiopathic Thrombocytopenia Purpura (ITP), IgA Nephropathy, Immunoproliferative Diseases or Disorders, Inflammatory Bowel Disease (IBD), Interstitial Lung Disease, Juvenile Arthritis, Juvenile Idiopathic Arthritis (JIA), Kawasaki's Disease, Lambert-Eaton Myasthenic Syndrome, Lichen Planus, Localized Scleroderma, Lupus Nephritis, Méniére's Disease, Microangiopathic Hemoytic Anemia, Microscopic Polyangitis, Miller Fischer Syndrome/Acute Disseminated Encephalomyeloradiculopathy, Mixed Connective Tissue Disease, Multiple Sclerosis (MS), Muscular Rheumatism, Myalgic Encephalomyelitis (ME), Myasthenia Gravis, Ocular Inflammation, Pemphigus Foliaceus, Pemphigus Vulgaris, Pernicious Anemia, Polyarteritis Nodosa, Polychondritis, Polyglandular Syndromes (Whitaker's syndrome), Polymyalgia Rheumatica, Polymyositis, Primary Agammaglobulinemia, Primary Biliary Cirrhosis/Autoimmune Cholangiopathy, Primary Glomerulonephritis, Primary Sclerosing Cholangitis, Psoriasis, Psoriatic Arthritis, Pure Red Cell Anemia, Raynaud's Phenomenon, Reiter's Syndrome/Reactive Arthritis, Relapsing Polychondritis, Restenosis, Rheumatic Fever, Rheumatic Disease, Rheumatoid Arthritis, Sarcoidosis, Schmidt's Syndrome, Scleroderma/Systemic Sclerosis, Sjörgen's Syndrome, Stiff-Man Syndrome, The Sweet Syndrome (Febrile Neutrophilic Dermatosis), Systemic Lupus Erythematosus (SLE), Systemic Scleroderma, Takayasu Arteritis, Temporal Arteritis/Giant Cell Arteritis, Thyroiditis, Type 1 diabetes, Type 2 diabetes, Uveitis, Vasculitis, Vitiligo, Wegener's Granulomatosis, and X-linked lymphoproliferative disease.

Of particular interest are diseases and disorders which are mainly driven by T-cell activation and proliferation, including:
diseases and disorders which are not linked to alloreactivity including:
   Alopecia areata, atopic dermatitis, eczema, psoriasis, lichen planus, psoriatic arthritis, vitiligo;
   Uveitis;
   Ankylosing spondylitis, Reiter's syndrome/reactive arthritis;
   Aplastic anemia, autoimmune lymphoproliferative syndrome/disorders, hemophagocytic lymphohistiocytosis;
   Type 1 diabetes; and
   Refractory celiac disease;
Acute rejection of grafted tissues and transplanted organs; acute graft versus host disease (GVHD) after transplantation of bone marrow cells or any other source of allogenic cells including hematopoietic precursors cells and/or stem cells.

Also of interest are diseases and disorders which are driven by both T- and B-cell activation and proliferation, with an important involvement of B-cells, including:
diseases and disorders for which the involvement of pathogenic auto-antibodies is well characterized, including:
   a Allergy;
   Cicatricial pemphigoid, bullous pemphigoid, epidermolysis bullosa acquisita, pemphigus foliaceus, pemphigus vulgaris, dermatitis herpetiformis;
   ANCA-associated vasculitis and microscopic polyangitis, vasculitis, Wegener's granulomatosis; Churg-Strauss syndrome (CSS), polyarteritis nodosa, cryoglobulin syndromes and essential mixed cryglobulinemia;
   Systemic lupus erythematosus (SLE), antiphospholipid syndrome (Hughes' syndrome), cutaneous lupus, lupus nephritis, mixed connective tissue disease; a Thyroiditis, Hashimoto thyroiditis, Grave's disease, exophthalmos;
   Autoimmune hemolytic anemia, autoimmune neutropenia, ITP, pernicious anaemia, pure red cell anaemia, micro-angiopathic hemolytic anemia;
   Primary glomerulonephritis, Berger's disease, Goodpasture's syndrome, IgA nephropathy; and
   Chronic idiopathic polyneuritis, chronic inflammatory demyelinating polyneuropathy (CIPD), chronic relapsing polyneuropathy (Guillain-Barré syndrome), Miller Fischer syndrome, Stiff man syndrome, Lambert-Eaton myasthenic syndrome, myasthenia gravis.
diseases and disorders for which the involvement of B-cells is less clearly characterized (although sometimes illustrated by the efficacy of anti-CD20 monoclonal antibodies or intravenous immunoglobulin infusions) and may not correspond or be limited to the production of pathogenic antibodies (nevertheless, non-pathogenic antibodies are sometimes described or even often present and used as a diagnosis biomarker), including:
   Addison's disease, autoimmune oophoritis and azoospermia, polyglandular syndromes (Whitaker's syndrome), Schmidt's syndrome;
   Autoimmune myocarditis, cardiomyopathy, Kawasaki's disease;
   Rheumatoid arthritis, Sjögren's syndrome, mixed connective tissue disease, polymyositis and dermatomyositis; polychondritis;
   Primary glomerulonephritis;
   Multiple sclerosis;
   Autoimmune hepatitis, primary biliary cirrhosis/autoimmune cholangiopathy,
   Hyper acute rejection of transplanted organs;
   Chronic rejection of graft or transplants;
   Chronic Graft versus Host reaction/disease after transplantation of bone marrow cells or hematopoietic precursor cells.

Additionally of interest are diseases and disorders for which the mechanism is shared between activation/proliferation of T-cells and activation/proliferation of innate immune cells and other inflammatory cellular subpopulations (including myeloid cells such as macrophages or granulocytes) and resident cells (such as fibroblasts and endothelial cells), including:
COPD, idiopathic pulmonary fibrosis, interstitial lung disease, sarcoidosis;
Adult onset Still's disease, juvenile idiopathic arthritis, Systemic sclerosis, CREST syndrome where B cells and pathogen antibodies may also play a role; the Sweet syndrome; Takayasu arteritis, temporal arteritis/giant cell arteritis;
Ulcerative cholangitis, inflammatory bowel disease (IBD) including Crohn's disease and ulcerative colitis, primary sclerosing cholangitis.

Also of interest are diseases and disorders for which the mechanism remains poorly characterized but involves the activation and proliferation of T-cells, including:
Alzheimer's disease, cardiovascular syndrome, type 2 diabetes, restenosis, chronic fatigue immune dysfunction syndrome (CFIDS).
Autoimmune Lymphoproliferative disorders, including:
Autoimmune Lymphoproliferative Syndrome and X-linked lymphoproliferative disease.

Suitably the disease or disorder is selected from: inflammatory skin diseases such as psoriasis or lichen planus; acute and/or chronic GVHD such as steroid resistant acute GVHD; acute lymphoproliferative syndrome; systemic lupus erythematosus, lupus nephritis or cutaneous lupus; or transplantation. In addition, the disease or disorder may be selected from myasthenia gravis, multiple sclerosis, and scleroderma/systemic sclerosis.

The compounds of formula (I) may be used in the treatment of cancer.

Thus, in one embodiment there is provided a compound of formula (I), or a pharmaceutically acceptable salt and/or solvate thereof and/or derivative thereof, for use in the treatment of cancer.

Further, there is provided a method for treating cancer in a subject, by administering to a subject in need thereof a compound of formula (I) or a pharmaceutically acceptable salt and/or solvate thereof and/or derivative thereof.

Additionally provided is the use of a compound of formula (I), or a pharmaceutically acceptable salt and/or solvate thereof and/or derivative thereof, in the manufacture of a medicament for the treatment of cancer in a subject.

Suitably the cancer is a haematological cancer, such as Acute myeloid leukemia, Angioimmunoblastic T-cell lymphoma, B-cell acute lymphoblastic leukemia, Sweet Syndrome, T-cell Non-Hodgkins lymphoma (including natural killer/T-cell lymphoma, adult T-cell leukaemia/lymphoma, enteropathy type T-cell lymphoma, hepatosplenic T-cell lymphoma and cutaneous T-cell lymphoma), T-cell acute lymphoblastic leukemia, B-cell Non-Hodgkins lymphoma (including Burkitt lymphoma, diffuse large B-cell lymphoma, Follicular lymphoma, Mantle cell lymphoma, Marginal Zone lymphoma), Hairy Cell Leukemia, Hodgkin lymphoma, Lymphoblastic lymphoma, Lymphoplasmacytic lymphoma, Mucosa-associated lymphoid tissue lymphoma, Multiple myeloma, Myelodysplastic syndrome, Plasma cell myeloma, Primary mediastinal large B-cell lymphoma, chronic myeloproliferative disorders (such as chronic myeloid leukemia, primary myelofibrosis, essential thrombocytemia, polycytemia vera) or chronic lymphocytic leukemia.

Alternatively, the cancer is a non-haematological cancer, such as selected from the group consisting of bladder cancer, breast, melanoma, neuroblastoma, malignant pleural mesothelioma, and sarcoma.

In addition, compounds of formula (I) may be used in enhancing recovery from vascular injury or surgery and reducing morbidity and mortality associated with neointima and restenosis in a subject. For example, the compounds of formula (I) may be used in preventing, reducing, or inhibiting neointima formation. A medical device may be treated prior to insertion or implantation with an effective amount of a composition comprising a compound of formula (I) in order to prevent, reduce, or inhibit neointima formation following insertion or implantation of the device or graft into the subject. The device can be a device that is inserted into the subject transiently, or a device that is implanted permanently. In some embodiments, the device is a surgical device.

Examples of medical devices include, but are not limited to, needles, cannulas, catheters, shunts, balloons, and implants such as stents and valves.

Suitably the subject is a mammal, in particular the subject is a human.

Pharmaceutical Compositions

In the following section, reference to compounds of formula (I) encompasses compounds of formula (I-a), (I-b) and (I-c).

For use in therapy the compounds of the invention are usually administered as a pharmaceutical composition. The invention also provides a pharmaceutical composition comprising a compound of formula (I), or a pharmaceutically acceptable salt and/or solvate (e.g. salt) and/or derivative thereof, and a pharmaceutically acceptable carrier or excipient.

In one embodiment, there is provided a pharmaceutical composition comprising a compound of formula (I), or a pharmaceutically acceptable salt and/or solvate (e.g. salt) and/or derivative thereof, for use in the treatment or prophylaxis of a disease or disorder as described herein.

In a further embodiment, there is provided a method for the prophylaxis or treatment of a disease or disorder as described herein, which comprises administering to a subject in need thereof an effective amount of a pharmaceutical composition comprising a compound of formula (I) or a pharmaceutically acceptable salt and/or solvate (e.g. salt) and/or derivative thereof.

The invention also provides the use of a pharmaceutical composition comprising a compound of formula (I), or a pharmaceutically acceptable salt and/or solvate thereof (e.g. salt) and/or derivative thereof, in the manufacture of a medicament for the treatment or prophylaxis of a disease or disorder as described herein.

The compounds of formula (I) or their pharmaceutically acceptable salts and/or solvates and/or derivatives thereof may be administered by any convenient method, e.g. by oral, parenteral, buccal, sublingual, nasal, rectal or transdermal administration, and the pharmaceutical compositions adapted accordingly.

The compounds of formula (I) or their pharmaceutically acceptable salts and/or solvates and/or derivatives thereof may be administered topically, for example to the eye, gut or skin. Thus, in an embodiment there is provided a pharmaceutical composition comprising a compound of the invention optionally in combination with one or more topically acceptable diluents or carriers.

A pharmaceutical composition of the invention may be delivered topically to the skin. Compositions suitable for transdermal administration include ointments, gels and patches. Such a pharmaceutical composition may also suitably be in the form of a cream, lotion, foam, powder, paste or tincture.

The pharmaceutical composition may suitably include vitamin D3 analogues (e.g. calcipotriol and maxacalcitol), steroids (e.g. fluticasone propionate, betamethasone valerate and clobetasol propionate), retinoids (e.g. tazarotene), coal tar and dithranol. Topical medicaments are often used in combination with each other (e.g. a vitamin D3 and a steroid) or with further agents such as salicylic acid.

A pharmaceutical composition of the invention may be delivered topically to the eye. Such a pharmaceutical composition may suitably be in the form of eye drops or an ointment.

A pharmaceutical composition of the invention may be delivered topically to the gut. Such a pharmaceutical composition may suitably be delivered orally, such as in the form of a tablet or a capsule, or rectally, such as in the form of a suppository.

Suitably, delayed release formulations are in the form of a capsule.

The compounds of formula (I) or their pharmaceutically acceptable salts and/or solvates and/or derivatives thereof which are active when given orally can be formulated as liquids or solids, e.g. as syrups, suspensions, emulsions, tablets, capsules or lozenges.

A liquid formulation will generally consist of a suspension or solution of the active ingredient (such as a compound of formula (I) or a pharmaceutically acceptable salt and/or solvate (e.g. salt) and/or derivative thereof) in a suitable liquid carrier(s) e.g. an aqueous solvent such as water, ethanol or glycerine, or a non-aqueous solvent, such as polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient (such as a compound of formula (I) or a pharmaceutically acceptable salt and/or solvate (e.g. salt) and/or derivative thereof) can be prepared using standard carriers and then filled into a hard gelatin capsule; alternatively a dispersion or suspension can be prepared using any suitable pharmaceutical carrier(s), e.g. aqueous gums, celluloses, silicates or oils and the dispersion or suspension then filled into a soft gelatin capsule.

Typical parenteral compositions consist of a solution or suspension of the active ingredient (such as a compound of formula (I) or a pharmaceutically acceptable salt and/or solvate (e.g. salt) and/or derivative thereof) in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomising device. Alternatively the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a fluoro-chloro-hydrocarbon or hydrofluorocarbon. Aerosol dosage forms can also take the form of pump-atomisers.

Compositions suitable for buccal or sublingual administration include tablets, lozenges and pastilles where the active ingredient is formulated with a carrier such as sugar and acacia, tragacanth, or gelatin and glycerin.

Compositions for rectal administration are conveniently in the form of suppositories containing a conventional suppository base such as cocoa butter.

Suitably, the composition is in unit dose form such as a tablet, capsule or ampoule.

The composition may for example contain from 0.1% to 100% by weight, for example from 10 to 60% by weight, of the active material, depending on the method of administration. The composition may contain from 0% to 99% by weight, for example 40% to 90% by weight, of the carrier, depending on the method of administration. The composition may contain from 0.05 mg to 2000 mg, for example from 1.0 mg to 500 mg, of the active material, depending on the method of administration. The composition may contain from 50 mg to 1000 mg, for example from 100 mg to 400 mg of the carrier, depending on the method of administration. The dose of the compound used in the treatment or prophylaxis of the aforementioned disorders will vary in the usual way with the seriousness of the disorders, the weight of the sufferer, and other similar factors. However, as a general guide suitable unit doses may be 0.05 mg to 1000 mg, more suitably 1.0 mg to 500 mg, and such unit doses may be administered more than once a day, for example two or three a day. Such therapy may extend for a number of weeks or months.

The invention provides, in a further aspect, a combination comprising a compound of formula (I) or a pharmaceutically acceptable, salt, solvate and/or derivative thereof (e.g. a combination comprising a compound of formula (I) or a pharmaceutically acceptable derivative thereof) together with a further pharmaceutically acceptable active ingredient or ingredients.

The invention provides a compound of formula (I), for use in combination with a further pharmaceutically acceptable active ingredient or ingredients.

When the compounds are used in combination with other therapeutic agents, the compounds may be administered separately, sequentially or simultaneously by any convenient route.

Optimal combinations may depend on the disease or disorder. Possible combinations include those with one or more active agents selected from the list consisting of: 5-aminosalicylic acid, or a prodrug thereof (such as sulfasalazine, olsalazine or bisalazide); corticosteroids (e.g. prednisolone, methylprednisolone, or budesonide); immunosuppressants (e.g. cyclosporin, tacrolimus, sirolimus, methotrexate, azathioprine mycophenolate mofetil, leflunomide, cyclophosphamide, 6-mercaptopurine or anti-lymphocyte (or thymocyte) globulins); anti-TNF-alpha antibodies (e.g., infliximab, adalimumab, certolizumab pegol or golimumab); anti-IL12/IL23 antibodies (e.g., ustekinumab); anti-IL6 or anti-IL6R antibodies, anti-IL17 antibodies or small molecule IL12/IL23 inhibitors (e.g., apilimod); Anti-alpha-4-beta-7 antibodies (e.g., vedolizumab); MAdCAM-1 blockers (e.g., PF-00547659); antibodies against the cell adhesion molecule alpha-4-integrin (e.g., natalizumab); antibodies against the IL2 receptor alpha subunit (e.g., daclizumab or basiliximab); JAK inhibitors including JAK1 and JAK3 inhibitors (e.g., tofacitinib, baricitinib, R348); Syk inhibitors and prodrugs thereof (e.g., fostamatinib and R-406); Phosphodiesterase-4 inhibitors (e.g., tetomilast); HMPL-004; probiotics; Dersalazine; semapimod/CPSI-2364; and protein kinase C inhibitors (e.g. AEB-071).

For cancer, the further pharmaceutically acceptable active ingredient may be selected from anti-mitotic agents such as vinblastine, paclitaxel and docetaxel; alkylating agents, for example cisplatin, carboplatin, dacarbazine and cyclophosphamide; antimetabolites, for example 5-fluorouracil, cytosine arabinoside and hydroxyurea; intercalating agents for example adriamycin and bleomycin; topoisomerase inhibitors for example etoposide, topotecan and irinotecan; thymidylate synthase inhibitors for example raltitrexed; PI3 kinase inhibitors for example idelalisib; mTor inhibitors for example everolimus and temsirolimus; proteasome inhibitors for example bortezomib; histone deacetylase inhibitors for example panobinostat or vorinostat; and hedgehog pathway blockers such as vismodegib.

The further pharmaceutically acceptable active ingredient may be selected from tyrosine kinase inhibitors such as, for example, axitinib, dasatinib, erlotinib, imatinib, nilotinib, pazopanib and sunitinib.

Anticancer antibodies may be included in a combination therapy and may be selected from the group consisting of olaratumab, daratumumab, necitumumab, dinutuximab, traztuzumab emtansine, pertuzumab, obinutuzumab, brentuximab, ofatumumab, panitumumab, catumaxomab, bevacizumab, cetuximab, tositumomab, traztuzumab, gentuzumab ozogamycin and rituximab.

Compounds or pharmaceutical compositions of the invention may also be used in combination with radiotherapy.

Some of the combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations. The individual components of combinations may also be administered separately, through the same or different routes.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

Medical Devices

In the following section, reference to compounds of formula (I) encompasses compounds of formula (I-a), (I-b) and (I-c).

In an embodiment, compounds of the invention or pharmaceutical compositions comprising said compounds may be formulated to permit incorporation into the medical device, thus providing application of the compound or composition directly to the site to prevent or treat conditions disclosed herein.

In an embodiment, the compounds of the invention or pharmaceutical composition thereof is formulated by including it within a coating onto the medical device. There are various coatings that can be utilized such as, for example, polymer coatings that can release the compound over a prescribed time period. The compound, or a pharmaceutical composition thereof, can be embedded directly within the medical device. In some embodiments, the compound is coated onto or within the device in a delivery vehicle such as a microparticle or liposome that facilitates its release and delivery. In some embodiments, the compound or pharmaceutical composition is miscible in the coating.

In some embodiments, the medical device is a vascular implant such as a stent. Stents are utilized in medicine to prevent or eliminate vascular restrictions. The implants may be inserted into a restricted vessel whereby the restricted vessel is widened. Excessive growth of the adjacent cells following vascular implantation results in a restriction of the vessel particularly at the ends of the implants which results in reduced effectiveness of the implants. If a vascular implant is inserted into a human artery for the elimination of for example an arteriosclerotic stenosis, intima hyperplasia can occur within a year at the ends of the vascular implant and results in renewed stenosis (“restenosis”).

Accordingly, in some embodiments, the stents are coated or loaded with a composition including a compound of the invention or pharmaceutical composition thereof and optionally a targeting signal, a delivery vehicle, or a combination thereof. Many stents are commercially available or otherwise know in the art.

In some embodiments, the stent is a drug-eluting stent. Various drug eluting stents that simultaneously deliver a therapeutic substance to the treatment site while providing artificial radial support to the wall tissue are known in the art. Endoluminal devices including stents are sometimes coated on their outer surfaces with a substance such as a drug releasing agent, growth factor, or the like. Stents have also been developed having a hollow tubular structure with holes or ports cut through the sidewall to allow drug elution from a central lumen. Although the hollow nature of the stent allows the central lumen to be loaded with a drug solution that is delivered via the parts or holes in the sidewall of the stent, the hollow tubular structure may not have suitable mechanical strength to provide adequate scaffolding in the vessel.

In some embodiments, the devices are also coated or impregnated with a compound of the invention, or pharmaceutical composition thereof and one or more additional therapeutic agents, including, but not limited to, antiplatelet agents, anticoagulant agents, anti-inflammatory agents, antimicrobial agents, antimetabolic agents, additional anti-neointima agents, additional antiproliferative agents, immunomodulators, antiproliferative agents, agents that affect migration and extracellular matrix production, agents that affect platelet deposition or formation of thrombis, and agents that promote vascular healing and re-endothelialization, such as those and others described in Sousa et al. (2003) and Salu et al. (2004).

Examples of antithrombin agents include, but are not limited to, Heparin (including low molecular heparin), R-Hirudin, Hirulog, Argatroban, Efegatran, Tick anticoagulant peptide, and Ppack.

Examples of antiproliferative agents include, but are not limited to, Paclitaxel (Taxol), QP-2 Vincristin, Methotrexat, Angiopeptin, Mitomycin, BCP 678, Antisense c-myc, ABT 578, Actinomycin-D, RestenASE, 1-Chlor-deoxyadenosin, PCNA Ribozym, and Celecoxib.

Examples of anti-restenosis agents include, but are not limited to, immunomodulators such as Sirolimus (Rapamycin), Tacrolimus, Biorest, Mizoribin, Cyclosporin, Interferon-γ Ib, Leflunomid, Tranilast, Corticosteroide, Mycophenolic acid and Biphosphonate.

Examples of anti-migratory agents and extracellular matrix modulators include, but are not limited to Halofuginone, Propyl-hydroxylase-Inhibitors, C-Proteinase-Inhibitors, MMP-Inhibitors, Batimastat, Probucol.

Examples of antiplatelet agents include, but are not limited to, heparin.

Examples of wound healing agents and endothelialization promoters include vascular epithelial growth factor (“VEGF”), 17-Estradiol, Tkase-Inhibitors, BCP 671, Statins, nitric oxide (“NO”)-Donors, and endothelial progenitor cell (“EPC”)-antibodies.

Besides coronary applications, drugs and active agents may be incorporated into the stent or stent coating for other indications. For example, in urological applications, antibiotic agents may be incorporated into the stent or stent coating for the prevention of infection. In gastroenterological and urological applications, active agents may be incorporated into the stent or stent coating for the local treatment of carcinoma. It may also be advantageous to incorporate in or on the stent a contrast agent, radiopaque markers, or other additives to allow the stent to be imaged in vivo for tracking, positioning, and other purposes. Such additives could be added to the absorbable composition used to make the stent or stent coating, or absorbed into, melted onto, or sprayed onto the surface of part or all of the stent. Preferred additives for this purpose include silver, iodine and iodine labelled compounds, barium sulfate, gadolinium oxide, bismuth derivatives, zirconium dioxide, cadmium, tungsten, gold tantalum, bismuth, platinum, iridium, and rhodium. These additives may be, but are not limited to, micro- or nano-sized particles or nano particles. Radio-opacity may be determined by fluoroscopy or by x-ray analysis.

A compound of the invention and one or more additional agents, or pharmaceutical composition thereof, can be incorporated into the stent, either by loading the compound and one or more additional agents, or pharmaceutical composition thereof into the absorbable material prior to processing, and/or coating the surface of the stent with the agent(s). The rate of release of agent may be controlled by a number of methods including varying the following: the ratio of the absorbable material to the compound and one or more additional agents, or pharmaceutical composition, the molecular weight of the absorbable material, the composition of the compound and one or more additional agents, or pharmaceutical composition, the composition of the absorbable polymer, the coating thickness, the number of coating layers and their relative thicknesses, and/or the compound and one or more additional agents, or pharmaceutical composition concentration. Top coats of polymers and other materials, including absorbable polymers, may also be applied to active agent coatings to control the rate of release. For example, P4HB can be applied as a top coat on a metallic stent coated with P4HB including an active agent to retard the release of the active agent.

The invention is further exemplified by the following non-limiting examples.

### EXAMPLES

Abbreviations used herein are defined below. Any abbreviations not defined are intended to convey their generally accepted meaning.

### Abbreviations

Ac acetyl (C(O)CH₃)
AcOH glacial acetic acid
AlMe₃ trimethylaluminium
aq aqueous
Ar Aromatic ring
BEH ethylene bridged hybrid
Bispin Bis(pinacolato)diboron; 4,4,4′,4′,5,5,5′,5′-Octamethyl-2,2′-bi-1,3,2-dioxaborolane
Bz benzyl (CH₂-phenyl)
Boc tert-butyloxycarbonyl protecting group
Cs₂CO₃ Cesium carbonate
CSH charged surface hybrid
d doublet
DABAL-Me₃ adduct of trimethylaluminum and 1,4-diazabicyclo[2.2.2]octane
DCM dichloromethane
DIPEA N, N-diisopropylethylamine
dioxane 1,4-dioxane
DMAP 4-dimethylaminopyridine
DME dimethoxyethane
DMF N,N-dimethylformamide
DMSO dimethyl sulfoxide
DMP Dess-Martin Periodinane
DPPA diphenylphosphoryl azide
dppf 1,1′-bis(diphenylphosphino)ferrocene
(ES⁺) electrospray ionisation, positive mode
(ES⁻) electrospray ionisation, negative mode
ESI electrospray ionisation
Et ethyl
EtI Ethyl iodide
EtOAc ethyl acetate
EtOH ethanol
g grams
Hal halogen
HATU 1-[bis(dimethylamino)methylene]-1H-1,2,3-triazolo[4,5-b]pyridinium 3-oxid hexafluorophosphate
HPLC high performance liquid chromatography
hr(s) hour(s)
IC₅₀ 50% inhibitory concentration
iPr iso-propyl
iPrMgCl iso-propyl magnesium chloride
K₂CO₃ potassium carbonate
LCMS liquid chromatography-mass spectrometry
LiHMDS lithium hexamethyldisilazide
LiOH lithium hydroxide
(M+H)⁺ protonated molecular ion
(M−H)⁻ unprotonated molecular ion
M molar concentration
mCPBA Meta-chloroperoxybenzoic acid
mL millilitre
mm millimiter
mmol millimole
Me methyl
MeCN acetonitrile
MeI iodomethane
MeOH methanol
MesCl methanesulfonyl chloride
MHz megahertz
min(s) minute(s)
MSD mass selective detector
m/z mass-to-charge ratio
N₂ nitrogen gas
NH₃ ammonia
NH₄Cl ammonium chloride
NaH sodium hydride
NaHCO₃ sodium bicarbonate
NaBH(OAc)₃ Sodium triacetoxyborohydride
nm nanometre
NMR nuclear magnetic resonance (spectroscopy)
NSFI N-fluorobenzenesulfonimide
P4HB poly-4-hydroxybutyrate
PDA photodiode array
Pd 170 chloro(crotyl)(2-dicyclohexylphosphino-2′,4′,6′-triisopropybiphenyl)palladium(II) or XPhos Pd(crotyl)Cl
Pd 174 allyl(2-di-tert-butylphosphino-2′,4′,6′-triisopropyl-1,1′-biphenyl)palladium(II) triflate or [tBuXPhosPd(allyl)]OTf
[Pd(allyl)Cl₂]₂ bis(allyl)dichlorodipalladium
PdCl₂(dppf) [1,1′-bis(diphenylphosphino)ferrocene]dichloropalladium(II)
Pd(PPh₃)₄ tetrakis(triphenylphosphine)palladium(0)
PMB 4-methoxybenzyl
prep HPLC preparative high performance liquid chromatography
Ph phenyl
pos/neg positive/negative
q quartet
RF/MS RapidFire Mass Spectrometry
RT room temperature
Rt retention time
RP reverse phase
s singlet
S_{N}Ar nucleophilic aromatic substitution
sat saturated
SCX solid supported cation exchange (resin)
Selectfluor N-chloromethyl-N′-fluorotriethylenediammonium bis(tetrafluoroborate)
t triplet
tBu tert-butyl
T3P 2,4,6-tripropyl-1,3,5,2,4,6-trioxatriphosphorinane-2,4,6-trioxide
TBME tert-butyl methyl ether
TFA Trifluoroacetic acid
[t-BuXPhos Pd(allyl)]OTf allyl(2-di-tert-butylphosphino-2′,4′,6′-triisopropyl-1,1′-biphenyl)palladium(II) triflate
THF tetrahydrofuran
TMP 2,2,6,6-tetramethylpiperidinyl
TMSOK potassium trimethylsilanolate
TTIP titanium tetraisopropoxide
UPLC ultra performance liquid chromatography
UV ultraviolet
v/v volume/volume
VWD variable wave detector
wt weight
um micrometre
uL microlitre
° C. degrees Celsius

### General Procedures

All starting materials and solvents were obtained either from commercial sources or prepared according to the literature. Unless otherwise stated all reactions were stirred. Organic solutions were routinely dried over anhydrous magnesium sulfate. Hydrogenations were performed on a Thales H-cube flow reactor under the conditions stated.

Column chromatography was performed on pre-packed silica (230-400 mesh, 40-63 um) cartridges using the amount indicated. SCX was purchased from Supelco and treated with 1M hydrochloric acid prior to use. Unless stated otherwise the reaction mixture to be purified was first diluted with MeOH and made acidic with a few drops of AcOH. This solution was loaded directly onto the SCX and washed with MeOH. The desired material was then eluted by washing with 0.7 M NH₃ in MeOH.

Preparative Reverse Phase High Performance Liquid Chromatography

Prep HPLC

Acidic Prep

Waters X-Select CSH column C18, 5 um (19×50 mm), flow rate 28 mL min⁻¹ eluting with a H₂O-MeCN gradient containing 0.1% v/v formic acid over 8.5 min using UV detection at 254 nm.

Basic Prep

Waters X-Bridge Prep column C18, 5 um (19×50 mm), flow rate 28 mL min⁻¹ eluting with a 10 mM NH₄HCO₃-MeCN gradient over 6.5 min using UV detection at 254 nm.

Analytical Methods

Reverse Phase HPLC Conditions for the LCMS Analytical Methods

HPLC acidic: Acidic LCMS 4 minute (5-95%)

Analytical LCMS was carried out using a Waters X-Select CSH C18, 2.5 um, 4.6×30 mm column eluting with a gradient of 0.1% Formic acid in MeCN in 0.1% Formic acid in water. The gradient from 5-95% 0.1% Formic acid in MeCN occurs between 0.00-3.00 minutes at 2.5 mL/min with a flush from 3.01-3.5 minutes at 4.5 mL/min. A column re-equilibration to 5% MeCN is from 3.60-4.00 minutes at 2.5 mL/min. UV spectra of the eluted peaks were measured using an Agilent 1260 Infinity VWD at 254 nm. Mass spectra were measured using an Agilent 6120 MSD running with positive/negative switching.

HPLC basic: Basic LCMS 4 minute (5-95%)

Analytical LCMS was carried out using a Waters X-Select BEH C18, 2.5 um, 4.6×30 mm column eluting with a gradient of MeCN in aqueous 10 mM ammonium bicarbonate. The gradient from 5-95% MeCN occurs between 0.00-3.00 minutes at 2.5 mL/min with a flush from 3.01-3.5 minutes at 4.5 mL/min. A column re-equilibration to 5% MeCN is from 3.60-4.00 minutes at 2.5 mL/min. UV spectra of the eluted peaks were measured using an Agilent 1260 Infinity VWD at 254 nm. Mass spectra were measured using an Agilent 6120 MSD running with positive/negative switching.

Reverse Phase HPLC Conditions for the UPLC Analytical Methods

UPLC acidic: Acidic UPLC 3 minute

Analytical UPLC/MS was carried out using a Waters Acquity CSH C18, 1.7 um, 2.1×30 mm column eluting with a gradient of 0.1% Formic acid in MeCN in 0.1% Formic acid in water. The gradient is structured with a starting point of 5% MeCN held from 0.0-0.11 minutes. The gradient from 5-95% occurs between 0.11-2.15 minutes with a flush from 2.15-2.56 minutes. A column re-equilibration to 5% MeCN is from 2.56-2.83 minutes. UV spectra of the eluted peaks were measured using an Acquity PDA and mass spectra were recorded using an Acquity QDa detector with ESI pos/neg switching.

Acidic UPLC 2 Acidic UPLC 1 Minute

Analytical UPLC/MS was carried out using a Waters Acquity CSH C18, 1.7 um, 2.1×30 mm column eluting with a gradient of 0.1% Formic acid in MeCN in 0.1% Formic acid in water. The gradient is structured with a starting point of 5% MeCN held from 0.0-0.08 minutes. The gradient from 5-95% occurs between 0.08-0.70 minutes with a flush from 0.7-0.8 minutes. A column re-equilibration to 5% MeCN is from 0.8-0.9 minutes. UV spectra of the eluted peaks were measured using an Acquity PDA and mass spectra were recorded using an Acquity QDa detector with ESI pos/neg switching.

UPLC Basic: Basic UPLC 3 Minute

Analytical UPLC/MS was carried out using a Waters Acquity BEH C18, 1.7 um, 2.1×30 mm column eluting with a gradient of MeCN in aqueous 10 mM Ammonium Bicarbonate. The gradient is structured with a starting point of 5% MeCN held from 0.0-0.11 minutes. The gradient from 5-95% occurs between 0.11-2.15 minutes with a flush from 2.15-2.56 minutes. A column re-equilibration to 5% MeCN is from 2.56-2.83 minutes. UV spectra of the eluted peaks were measured using an Acquity PDA and mass spectra were recorded using an Acquity QDa detector with ESI pos/neg switching.

Basic UPLC 2 Basic UPLC 1 minute Analytical UPLC/MS was carried out using a Waters Acquity BEH C18, 1.7 um, 2.1×30 mm column eluting with a gradient of MeCN in aqueous 10 mM Ammonium Bicarbonate. The gradient is structured with a starting point of 5% MeCN held from 0.0-0.08 minutes. The gradient from 5-95% occurs between 0.08-0.70 minutes with a flush from 0.7-0.8 minutes. A column re-equilibration to 5% MeCN is from 0.8-0.9 minutes. UV spectra of the eluted peaks were measured using an Acquity PDA and mass spectra were recorded using an Acquity QDa detector with ESI pos/neg switching.

Column temperature is 40° C. in all runs. Injection volume is 3 uL and the flow rate is 0.77 mL/min.

PDA scan from 210-400 nm on all runs.

Normal Phase HPLC Conditions for the Chiral Analytical Methods

Chiral IC3 method: Chiral HPLC (Diacel Chiralpak IC, 5 um, 4.6×250 mm, 1.0 mL/min, 25-70% EtOH (0.2% TFA) in iso-hexane (0.2% TFA)

Chiral IC4 method: Chiral HPLC (Diacel Chiralpak IC, 5 um, 4.6×250 mm, 1.0 mL/min, 40% EtOH (0.2% TFA) in 4:1 heptane/chloroform (0.2% TFA).

Chiral IC5 method: Chiral HPLC (Diacel Chiralpak IC, 5 um, 4.6×250 mm, 1.0 mL/min, 20% EtOH (0.2% TFA) in iso-hexane (0.2% TFA).

Reverse Phase HPLC Conditions for the Chiral Analytical Methods

Chiral IC6 method: Chiral HPLC (Diacel Chiralpak IC, 5 um, 4.6×250 mm, 1.0 mL/min, 50% MeCN (0.1% formic acid) in water (0.1% formic acid).

Chiral IC7 method: Chiral HPLC (Diacel Chiralpak IC, 5 um, 4.6×250 mm, 1.0 mL/min, 5-95% MeCN (0.1% formic acid) in water (0.1% formic acid).

¹H NMR Spectroscopy

¹H NMR spectra were acquired on a Bruker Avance III spectrometer at 400 MHz or Bruker Avance III HD spectrometer at 500 MHz using residual undeuterated solvent as reference and unless specified otherwise were run in DMSO-d6.

### Preparation of Intermediates

Known synthetic intermediates were procured from commercial sources or were obtained using published literature procedures. Additional intermediates were prepared by the representative synthetic processes described herein.

Compounds of Formula (I-a)—Intermediates

Any one of Methods 1-1q (referred to later herein) or A-N and Q-R may be used in the synthesis of the compounds of formula (I). For example, a scheme which is shown using a compound wherein X=N, Y=CR₂ and Z=CR₃ may also be used in the synthesis of compounds wherein X, Y and Z are as defined in the claims.

The synthesis of INTC1 to INTC179 and INTD1 to INTD86 is disclosed in at least one of WO02019/179852, VNO2019/180244 and VNO2020/083975, each of which is herein incorporated in its entirety by reference.

Method A: Decarboxylation of Chloro-Heterocycles Such as Chloro-Pyrimidines

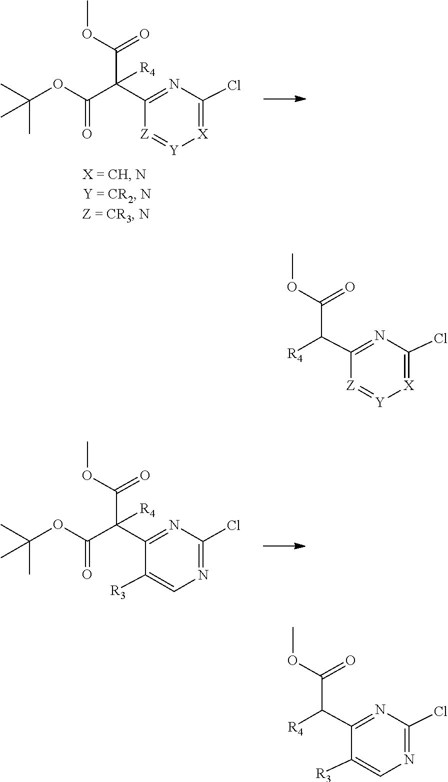

TFA (10 eq) was added dropwise to an ice-cooled, stirred solution of malonate derivative (1 eq) in DCM (15 volumes). The reaction vessel was stirred at RT for 18 hrs and then concentrated. The crude product was purified by normal phase chromatography.

Method B: Alkylation

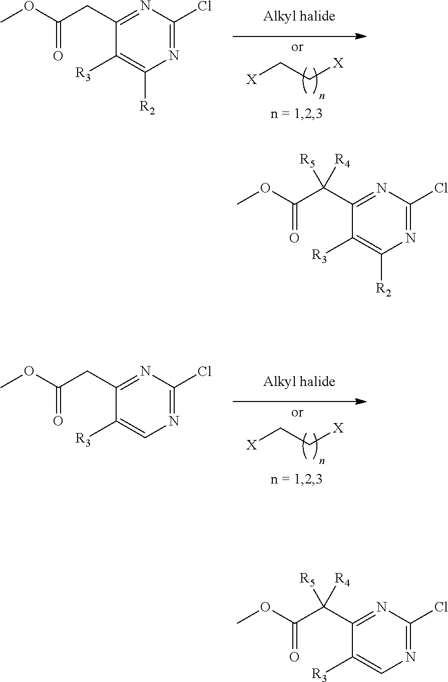

Base (2.5-5 eq) was added to an ice-cooled, stirred mixture of methyl 2-(2-chloropyrimidin-4-yl)acetate (1 eq) in appropriate polar aprotic solvent such as DMF or acetone (10 volumes). After 20 min, alkyl halide (1-5 eq) was added. The reaction vessel was stirred at 0° C. for 30 mins then at RT for 2 hrs. The reaction was quenched with NH₄Cl (aq) or 1M HCl (aq), stirred for 20 mins then extracted with EtOAc. The organic phases were died (phase separator) and concentrated. The crude product was purified by normal phase chromatography.

Method C: Formation of Sulfonamides from Aromatic Halides

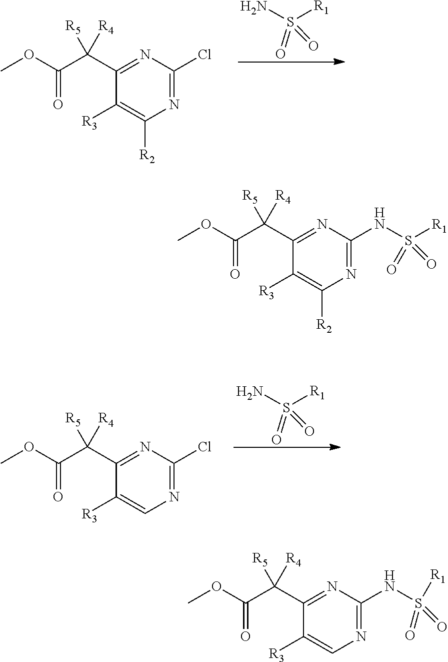

2-Chloropyrimidine intermediate (1 eq), sulfonamide (1.2 eq) and base (2 eq) were dissolved in dioxane (40 volumes). The mixture was degassed (N₂, 5 mins) then catalyst (5 mol %) was added. The resulting mixture was heated under nitrogen at 90° C. for 2 hrs. The mixture was filtered, washing with EtOAc or DCM and the resulting filtrate was concentrated. The crude product was purified by normal phase chromatography or trituration using a suitable solvent.

Method D: Decarboxylation of Pyrimidines Bearing Sulfonamides

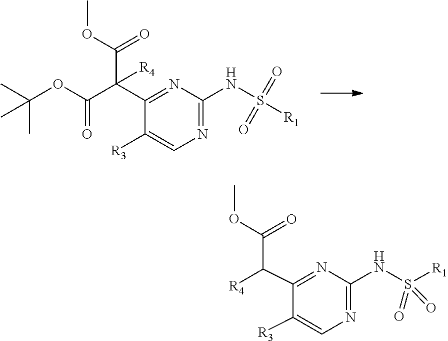

TFA (10 eq) was added dropwise to an ice-cooled, stirred solution of malonate derivative (1 eq) in DCM (15 volumes). The reaction vessel was stirred at RT for 18 hrs and then concentrated. The crude product was purified by normal phase chromatography.

Method H: Benzylic Fluorination of Hetero-Aromatic Esters

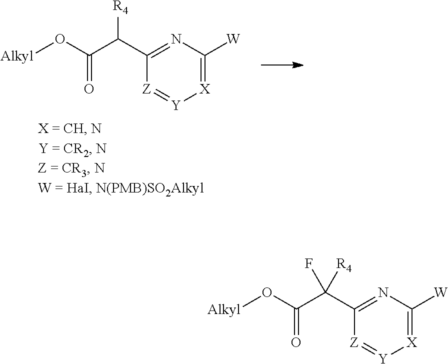

A solution of hetero-aromatic ester (1 eq) in THF (10 volumes) was cooled to −78° C. to which was added LiHMDS (1.25 eq 1M in THF). The reaction mixture was then warmed to RT for 1 hr. The solution was cooled to −78° C. and a solution (in THF) of, or solid, NSFI (1.25 eq) was added dropwise then warmed to RT for 2 hrs. The solution was diluted with sat NaHCO₃ (aq) and the product was extracted into EtOAc. The crude product was purified by normal phase chromatography.

Method I: Buchwald Coupling—Sulfonylation

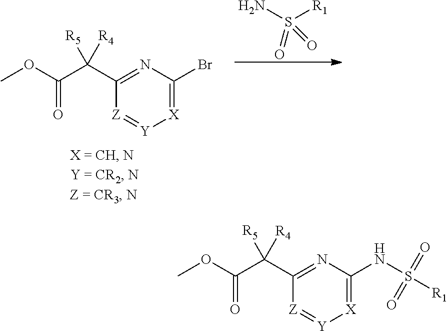

2-Bromopyridine intermediate (1 eq), sulfonamide (1.2 eq) and base (2 eq) were dissolved in dioxane (40 volumes). The mixture was degassed (N₂, 5 mins) then catalyst (5 mol %) was added. The resulting mixture was heated under nitrogen at 90° C. for 2 hrs. The mixture was filtered, washing with EtOAc or DCM and the resulting filtrate was concentrated. The crude product was purified by normal phase chromatography.

Method J: Hydrolysis

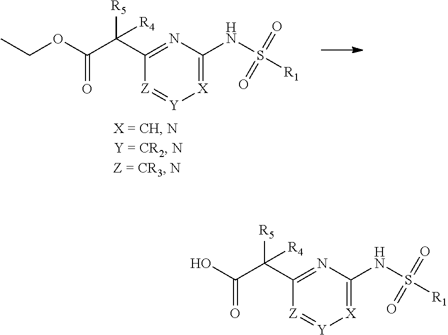

2M LiOH (aq, 2 eq) was added into a solution of ester (1 eq) in MeOH (3 volumes) and THF (3 volumes) and the resulting reaction mixture was stirred at 50° C. for 2 hrs. The solvent was removed under reduced pressure and then was acidified with 1M HCl (aq) until pH 3. The solution was extracted with EtOAc, the organic phase was passed through a phase separator and the solvent was removed. The compound was used crude or purified by reverse phase chromatography.

### Amine Intermediate Preparation

Method E: Suzuki Coupling of Halo Anilines with Heteroaromatic Boronates

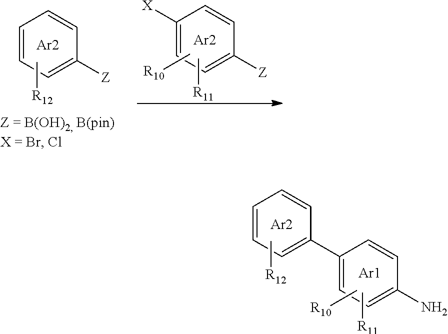

A solution of Ar1-X (1 eq) and Ar2-Z (1 eq) in solvent (3 volumes) and base (2.5 eq) was degassed (N₂, 5 min) and heated to 40° C. whereupon Pd catalyst (3 mol %) was added and the reaction mixture further degassed (N₂, 5 min) before being heated to 90° C. for 90 mins. The reaction mixture was allowed to cool to RT. In general, the desired compound was purified by column chromatography.

Method F: Suzuki Coupling of Heteroaromatic Halides with Aniline Boronates

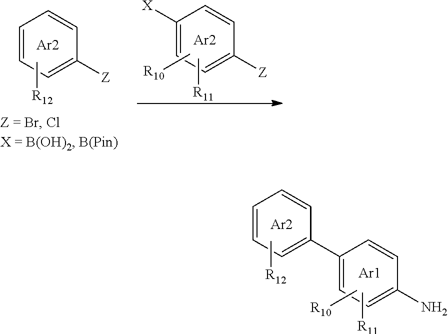

Pd catalyst (5 mol %) was added to a degassed (N₂, 5 mins) solution of Ar1-X (1 eq), Ar2-Z (1 eq) and base (3 eq, 6.85 mmol) in solvent (3 volumes). The solution was then degassed further (N₂, 5 mins) and then heated to 90° C. for 2 hrs then allowed to cool to RT. In general, the desired compound was purified by column chromatography.

Method G: Telescoped Boronate Formation and Suzuki Coupling

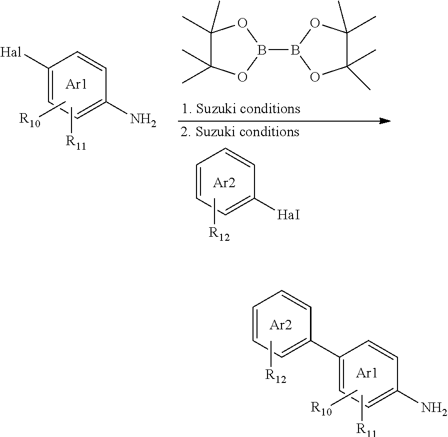

Bispin (1.1 eq) and KOAc (4 eq) were added to Ar1-Hal (1 eq) in dioxane (5 volumes). The reaction was heated to 60° C. and degassed (N₂, 5 mins). PdCl₂(dppf) (5 mol %) was added to the reaction mixture and the temperature was increased to 90° C. for 1 hr. The reaction mixture was then cooled to RT and a solution of Ar2-Hal (1 eq) in dioxane (3 volumes) was added, followed by a solution of K₂CO₃ (4 eq) in water (2 volumes). The temperature was then increased to 90° C. for 18 hrs. The reaction was cooled to RT, an aqueous work up was performed and the crude compound was purified by normal phase chromatography.

Anilines

Method K: Suzuki Coupling

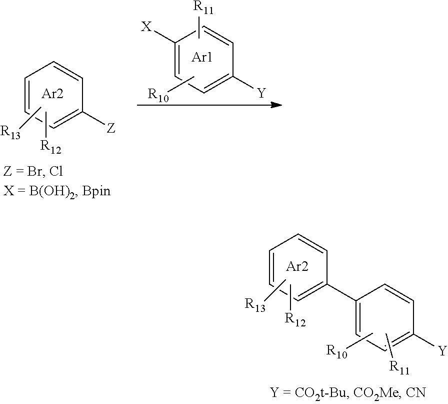

A solution of boronic acid (1 eq), aryl halide (1.05 eq.) and Cs₂CO₃ (3 eq.) in a mixture of dioxane (40 volumes) and water (6 volumes) was degassed (N₂, 5 mins). PdCl₂(dppf).CH₂Cl₂ (5 mol %) was added and the reaction was further degassed (N₂) before being heated to 90° C. for 18 hrs. The reaction mixture was filtered through celite before an aqueous workup was undertaken, followed by purification by normal phase chromatography.

Method L: Ester Deprotection with TFA

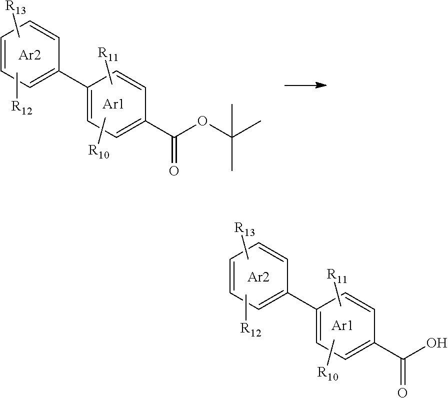

A solution of the ester (1 eq) in DCM (20 volumes) was treated with TFA (10 eq.) and stirred at RT for 3 hrs. The reaction mixture was then concentrated and azeotroped with MeOH and MeCN. No further purification was undertaken.

Method M: Ester Deprotection with Base

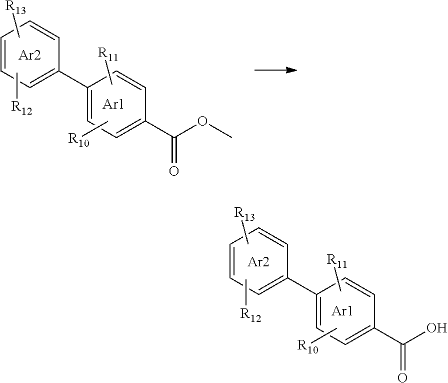

A solution of the ester (1 eq) in a mixture of THF/MeOH (4/1 volumes) was treated with LiOH (2.2-6 eq.) and stirred between RT and 50° C. for between 3 hrs and 18 hrs. The organic solvents were removed in vacuo then acidified with 1 M HCl and extracted with EtOAc. The organic phases were combined, dried (Na₂SO₄), filtered and concentrated. The products were used directly in the next step with no further purification undertaken.

Method N: Potassium Salt Formation

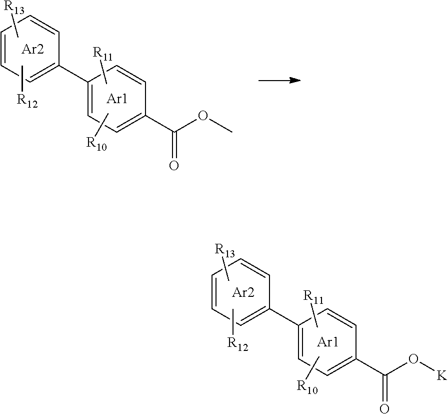

A solution of the ester (1 eq.) in THF (4 volumes) was treated with TMSOK (1 eq.) and stirred at RT for 2 hrs before the reaction mixtures were filtered and washed with iso-hexanes. The products were used directly in the next step with no further purification undertaken.

Method P: SNAR using 4-chloro-2-(methylthio)-heterocycles

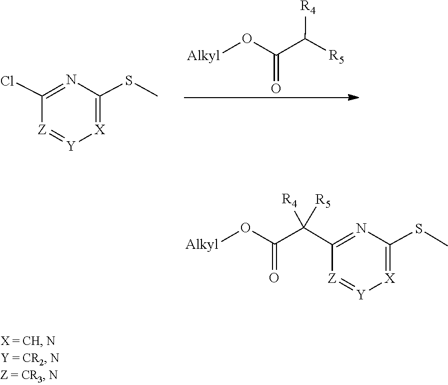

A solution of hetero-aromatic chloride (1 eq) and ester (1 eq) in THF (5-20 volumes) was warmed to 30° C. to which was added UHMDS (1.25 eq 1-1.5M in THF). The reaction mixture was stirred at this temperature for up to 3 hrs, then was poured into water and extracted with EtOAc. The organic extract was washed with brine, dried (MgSO₄), filtered and the solvent removed in vacuo to afford the desired compound. If required, the crude product was purified by normal phase chromatography.

TABLE 1
The following intermediates were made according to Method P.

| | Name/Structure | Synthesis | |
|---|---|---|---|
| | (All examples containing | Method, [LCMS | |
| | chiral centres are | Method], m/z | ¹H NMR Chemical Shift Data |
| INTC | racemates unless stated) | (M + H)⁺, (Rt/min) | (DMSO-d6 unless stated) |
| INTC186 | 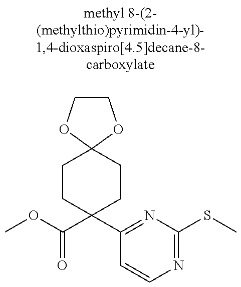 | Method P, [HPCL acidic], 325 (1.99). | 8.60 (d, J = 5.3 Hz, 1H), 7.24 (d, J = 5.3 Hz, 1H), 3.88-3.86 (m, 4H), 3.65 (s, 3H), 2.49 (s, 3H), 2.30-2.24 (m, 2H), 2.16-2.07 (m, 2H), 1.65-1.56 (m, 4H). |
| INTC187 | 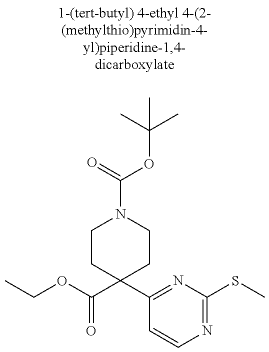 [SMILES: CCOC(=O)C1(c2ccnc(SC)n2)CCN(C(=O)OC(C)(C)C)CC1] | Method P, [HPLC acidic], 382 (2.54). | 8.61 (d, J = 5.3 Hz, 1H), 7.25 (d, J = 5.3 Hz, 1H), 4.14 (q, J = 7.1 Hz, 2H), 3.73-3.65 (m, 2H), 3.32 (s, 1H), 2.49 (s, 3H), 2.26- 2.20 (m, 1H), 2.02-1.93 (m, 2H), 1.40 (s, 9H), 1.14 (t, J = 7.1 Hz, 3H), 1H obscured by DMSO and 1H by obscured by water |
| INTC188 | 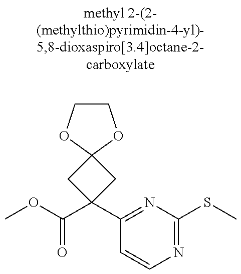 | Method P, [HPLC acidic], 297 (1.76). | No data collected |
| INTC190 | 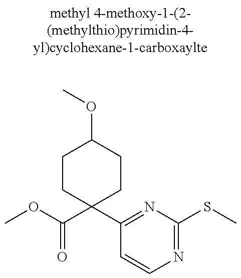 | Method P, [HPLC acidic], 297 (2.00). | 8.63-8.56 (m, 1H), 7.22 (d, J = 5.2 Hz, 1H), 3.65 (s, 3H), 3.24 (s, 3H), 3.23-3.20 (m, 1H), 2.48 (s, 3H), 2.40-2.28 (m, 2H), 1.93-1.80 (m, 4H), 1.36-1.24 (m, 2H). |
| INTC236 | 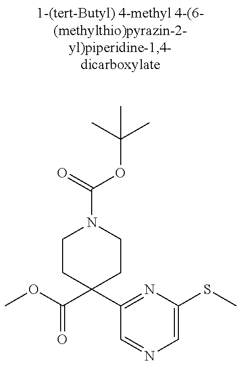 | Method P, [HPLC acidic], (M − Boc) + H 268, (1.58) | 8.53 (s, 1H), 8.40 (s, 1H), 3.72- 3.59 (m, 2H), 3.66 (s, 3H), 3.24- 3.00 (m, 2H), 2.52 (s, 3H), 2.25-2.30 (m, 2H), 2.00-2.10 (m, 2H), 1.40 (s, 9H). |
| INTC241 | 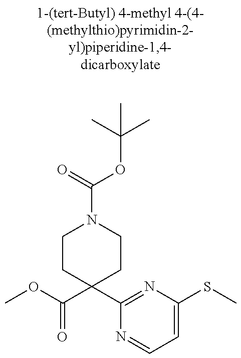 | Method P, [HPLC Acidic], (M − tBu) + H 312, (1.57) | 8.45 (d, J = 5.5 Hz, 1H), 7.35 (d, J = 5.5 Hz, 1H), 3.64 (s, 3H), 3.62-3.55 (m, 2H), 2.53-2.47 (m, 2H), 2.21-2.09 (m, 4H), 1.40 (s, 9H). 3H not observed, obscured by DMSO peak. |

### Ethyl 4-(2-(methylthio)pyrimidin-4-yl)piperidine-4-carboxylate INTC191

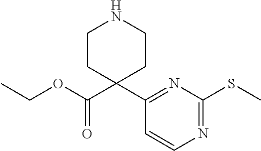 [SMILES: CCOC(=O)C1(c2ccnc(SC)n2)CCNCC1]

To a solution of 1-tert-butyl 4-ethyl 4-(2-(methylthio)pyrimidin-4-yl)piperidine-1,4-dicarboxylate (4 g, 9.44 mmol) INTC187 in DCM (30 mL) at RT was added TFA (5 mL). The reaction mixture was stirred at RT for 1 hr. Additional TFA (5 mL) added and the reaction was stirred at RT for a further 1 hr. The reaction mixture was quenched by addition of NaHCO₃ (aq, 100 mL), gas evolved, and was diluted with DCM (50 mL). The organics were isolated and dried (MgSO₄), filtered and solvent removed in vacuo to afford ethyl 4-(2-(methylthio)pyrimidin-4-yl)piperidine-4-carboxylate (2.6 g, 9.15 mmol, 97% yield) as a brown oil. Rt 0.97 min (HPLC, acidic); m/z 282 (M+H)⁺ (ES+); No NMR data collected.

### Ethyl 1-(methylsulfonyl)-4-(2-(methylthio)pyrimidin-4-yl)piperidine-4-carboxylate INTC192

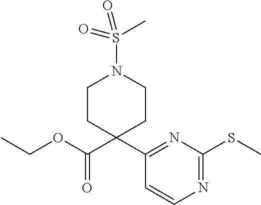 [SMILES: CCOC(=O)C1(c2ccnc(SC)n2)CCN(S(C)(=O)=O)CC1]

To a stirred solution of ethyl 4-(2-(methylthio)pyrimidin-4-yl)piperidine-4-carboxylate (1.3 g, 4.62 mmol) INTC191 in DCM (15 mL) at RT was added TEA (1.42 mL, 10.16 mmol) and then MesCl (0.37 mL, 5.08 mmol). After 1 hr, 1 M HCl (aq, 50 mL) and DCM (30 mL) were added. The organic layer was isolated by passage through a phase separation cartridge and then concentrated in vacuo to afford ethyl 1-(methylsulfonyl)-4-(2-(methylthio)pyrimidin-4-yl)piperidine-4-carboxylate (1.21 g, 3.37 mmol, 73% yield) as a brown oil. Rt 1.93 min (HPLC, acidic); m/z 360 (M+H)⁺ (ES+); No NMR data collected.

Amide Formation

### (4-(2-(Methylthio)pyrimidin-4-yl)tetrahydro-2H-pyran-4-yl)methanol INTC197

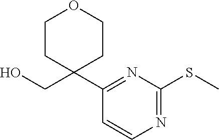 [SMILES: CSc1nccc(C2(CO)CCOCC2)n1]

LiCl (0.95 g, 22.4 mmol) followed by NaBH₄ (0.85 g, 22.4 mmol) and EtOH (15 mL) was added into a stirring solution of methyl 4-(2-(methylthio)pyrimidin-4-yl)tetrahydro-2H-pyran-4-carboxylate INTC178 (3 g, 11.2 mmol) in THF (15 mL). The resulting reaction mixture was stirred at RT for 18 hrs. The reaction mixture was acidified with 1M HCl (aq, 20 mL) and the volatiles were removed in vacuo. The residue was extracted with DCM (3×150 mL). The organic extract was dried (MgSO₄), filtered and solvent removed in vacuo. The crude product was purified by chromatography on silica gel (40 g column, 0-100% EA/iso-hexanes) to afford (4-(2-(methylthio)pyrimidin-4-yl)tetrahydro-2H-pyran-4-yl)methanol (2.3 g, 9.09 mmol, 81% yield) as a colourless gum. Rt 0.80 min (UPLC acidic); m/z 241 (M+H)⁺ (ES+). ¹H NMR (500 MHz, DMSO-d6) δ 8.54 (d, J=5.3 Hz, 1H), 7.23 (d, J=5.3 Hz, 1H), 4.73 (t, J=5.6 Hz, 1H), 3.74-3.67 (m, 2H), 3.49 (d, J=5.7 Hz, 2H), 3.35-3.27 (m, 2H), 2.50 (s, 3H), 2.19-2.10 (m, 2H), 1.77-1.67 (m, 2H).

### 4-(2-(Methylthio)pyrimidin-4-yl)tetrahydro-2H-pyran-4-carbaldehyde INTC198

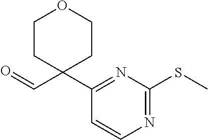 [SMILES: CSc1nccc(C2(C=O)CCOCC2)n1]

DMP (1.77 g, 4.16 mmol) was added portionwise into a stirring solution of (4-(2-(methylthio)pyrimidin-4-yl)tetrahydro-2H-pyran-4-yl)methanol INTC197 (1 g, 4.16 mmol) in DCM (25 ml). The resulting reaction mixture was stirred at RT for 1 hr. The reaction mixture was poured into sat. NaHCO₃ (aq, 100 mL) and extracted with DCM (3×100 mL). The organic extract was sequentially washed with saturated sat. NaHCO₃ (aq, 100 mL), and brine (100 mL). The organic extract was dried (MgSO₄), filtered and solvent in vacuo to afford 4-(2-(methylthio)pyrimidin-4-yl)tetrahydro-2H-pyran-4-carbaldehyde (900 mg, 3.40 mmol, 82% yield) as a colorless oil. Rt 1.61 min (HPLC acidic); m/z 239 (M+H)⁺ (ES⁺). ¹H NMR (500 MHz, DMSO-d6) δ 9.63 (s, 1H), 8.65 (d, J=5.2 Hz, 1H), 7.30 (d, J=5.2 Hz, 1H), 3.68-3.59 (m, 2H), 3.56-3.48 (m, 2H), 2.51 (s, 3H), 2.28-2.20 (m, 2H), 2.16-2.09 (m, 2H).

### 4-(6-Ethoxypyrazin-2-yl)-N-((4-(2-(methylthio)pyrimidin-4-yl)tetrahydro-2H-pyran-4-yl)methyl)aniline INTC199

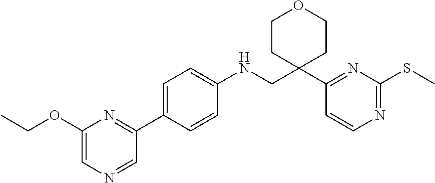 [SMILES: CCOc1cncc(-c2ccc(NCC3(c4ccnc(SC)n4)CCOCC3)cc2)n1]

NaBH(OAc)₃ (1.07 g, 5.04 mmol) was added into a suspension of 4-(2-(methylthio)pyrimidin-4-yl)tetrahydro-2H-pyran-4-carbaldehyde INTC198 (400 mg, 1.68 mmol) and 4-(6-ethoxypyrazin-2-yl)aniline INTD18 (542 mg, 2.52 mmol) in DCM (10 ml) and the resulting reaction mixture was stirred at ambient temperature for 18 hrs. The reaction mixture was diluted with DCM (100 mL) and sequentially washed with sat. NaHCO₃ (aq, 2×100 mL) and brine (100 mL), dried (MgSO₄), filtered and solvent removed in vacuo. The crude product was purified by chromatography on silica gel (25 g cartridge, 0-100% EtOAc/iso-hexanes) to afford 4-(6-ethoxypyrazin-2-yl)-N-((4-(2-(methylthio)pyrimidin-4-yl)tetrahydro-2H-pyran-4-yl)methyl)aniline (312 mg, 0.706 mmol, 42% yield) as a yellow gum. Rt 2.49 min (HPLC acidic); m/z 438 (M+H)⁺ (ES⁺). ¹H NMR (500 MHz, DMSO-d6) δ 8.57 (s, 1H), 8.52 (d, J=5.2 Hz, 1H), 7.99 (s, 1H), 7.81-7.74 (m, 2H), 7.29 (d, J=5.3 Hz, 1H), 6.66-6.57 (m, 2H), 6.00 (t, J=6.6 Hz, 1H), 4.42 (q, J=7.0 Hz, 2H), 3.80-3.70 (m, 2H), 3.38 (d, J=6.6 Hz, 2H), 3.32-3.26 (m, 2H), 2.52 (s, 3H), 2.32-2.26 (m, 2H), 1.89-1.80 (m, 2H), 1.38 (t, J=7.0 Hz, 3H).

Method Q: Oxidation of Thioethers to Sulfones or Sulfoxides

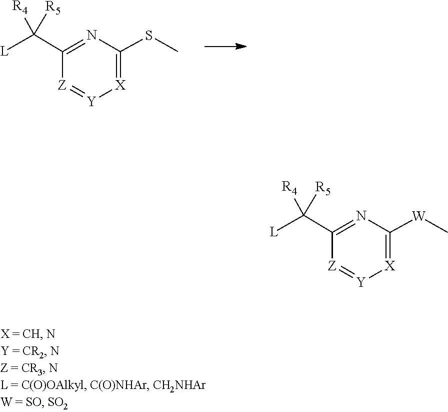

mCPBA (2.2 eq) was portionwise to a stirred solution of thiother (1 eq) in DCM (20-50 volumes) maintaining the internal temperature at RT. The resulting mixture was stirred at RT for a further 3 hrs. The reaction mixture was poured into sat aq. Na₂SO₃ and extracted with DCM. The organic extract was sequentially washed with sat aq. NaHCO₃ and brine, dried (MgSO₄), filtered and solvent removed in vacuo to afford the desire compound.

TABLE 2
The following intermediates were made according to Method Q.

| | | Synthesis | |
|---|---|---|---|
| | | Method, | |
| | Name/Structure | [LCMS | |
| | (All examples containing chiral | Method], m/z | |
| | centres are racemates unless | (M + H)⁺, | ¹H NMR Chemical Shift Data |
| INTC | stated) | (Rt/min) | (DMSO-d6 unless stated) |
| INTC203 | 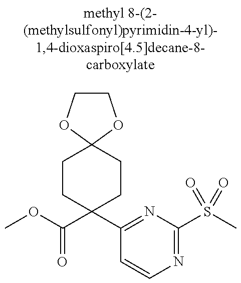 | Method Q using INTC186, [HPLC acidic], 357 (1.49). | 9.06 (d, J = 5.3 Hz, 1H), 7.93 (d, J = 5.3 Hz, 1H), 3.91-3.83 (m, 4H), 3.66 (s, 3H), 3.41 (s, 3H), 2.37-2.29 (m, 2H), 2.29- 2.18 (m, 2H), 1.68-1.56 (m, 4H). |
| INTC207 | 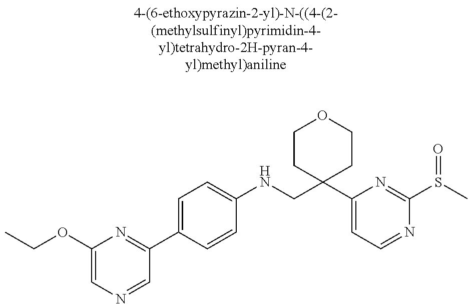 [SMILES: CCOc1cncc(-c2ccc(NCC3(c4ccnc(S(C)=O)n4)CCOCC3)cc2)n1] | Method Q using INTC199, [HPLC, acidic], 454 (1.89). | 8.87 (d, J = 5.3 Hz, 1H), 8.56 (s, 1H), 7.99 (s, 1H), 7.77- 7.71 (m, 3H), 6.57-6.47 (m, 2H), 6.07 (t, J = 6.7 Hz, 1H), 4.41 (q, J = 7.0 Hz, 2H), 3.80- 3.74 (m, 2H), 3.47-3.42 (m, 2H), 3.31-3.24 (m, 2H), 2.83 (s, 3H), 2.36 (d, J = 13.4 Hz, 2H), 1.95-1.84 (m, 2H), 1.37 (t, J = 7.0 Hz, 3H). |
| INTC208 | 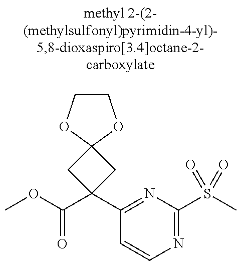 | Method Q using INTC188, [HPLC, acidic], 329 (1.27). | No data collected |
| INTC210 | 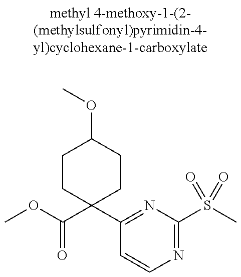 | Method Q using INTC190, [HPLC acidic], 329 (1.46). | No data collected |
| INTC233 | 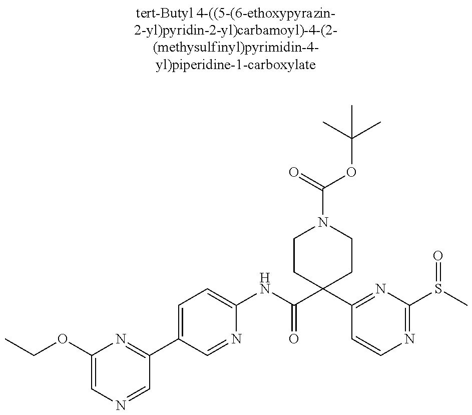 [SMILES: CCOc1cncc(-c2ccc(NC(=O)C3(c4ccnc(S(C)=O)n4)CCN(C(=O)OC(C)(C)C)CC3)nc2)n1] | Method Q using INTC232, [HPLC Acidic], 568, (2.23) | No NMR recorded |
| INTC237 | 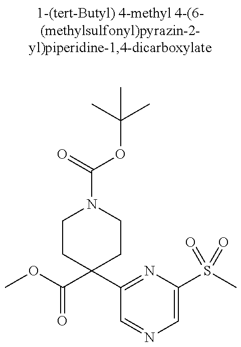 | Method Q using INTC236, [HPLC acidic], (M − Boc) + H 300, (1.32) | 9.19 (s, 1H), 9.17 (s, 1H), 3.67 (s, 3H), 3.59-3.46 (m, 2H), 3.35 (s, 3H), 3.30-3.20 (m, 2H), 2.36-2.28 (m, 2H), 2.28- 2.19 (m, 2H), 1.40 (d, J = 5.3 Hz, 9H). |
| INTC242 | 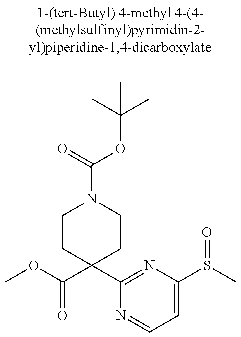 | Method Q using INTC241, [UPLC Acidic], (M − Boc) + H 284, (0.57) | No NMR recorded |

Method R: Formation of Sulfonamides from Aromatic Sulfones

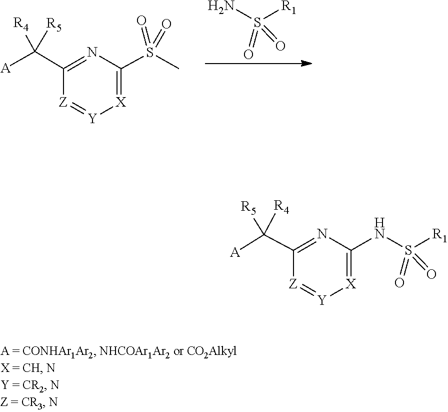

To a solution of sulfone (1.0 eq) and primary sulfonamide (1.1-2.0 eq) in polar aprotic solvent such as NMP (5-100 volumes) was added an inorganic base (3 eq) such as cesium carbonate and heated to 40-90° C. for 1-3 hrs. The reaction mixture was cooled to RT and diluted with water (50-100 volumes) and the mixture was washed with MTBE (100 volumes) and the aqueous was slowly acidified to pH 5 or lower using an appropriate acid such as HCl. The resulting precipitate was filtered to afford desired sulfonamide product.

TABLE 3
The following intermediates were made according to Method R.

| | | Synthesis | | |
|---|---|---|---|---|
| | | Method, | | |
| | | [LCMS | | |
| | | Method], | ¹H NMR Chemical Shift | |
| | Name/Structure | m/z | Data | |
| | (All examples containing chiral | (M + H)⁺, | (DMSO-d6 unless | Base, |
| INTC | centres are racemates unless stated) | (Rt/min) | stated) | Solvent |
| INTC214 | 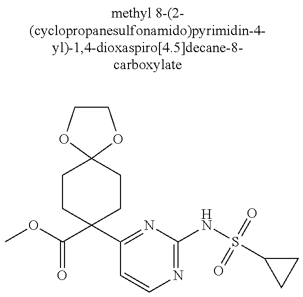 [SMILES: COC(=O)C1(c2ccnc(NS(=O)(=O)C3CC3)n2)CCC2(CC1)OCCO2] | Method R using INTC203, [HPLC acidic], 398 (1.63). | 11.28 (s, 1H), 8.63-8.54 (m, 1H), 7.21-7.11 (m, 1H), 3.87 (s, 3H), 3.65 (s, 4H), 3.24-3.15 (m, 1H), 2.31-2.23 (m, 2H), 2.18- 2.02 (m, 2H), 1.70-1.52 (m, 4H), 1.19-0.99 (m, 4H). | Cs₂CO₃, NMP |
| INTC215 | 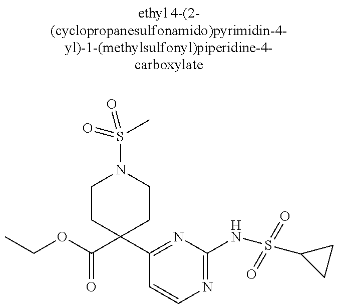 [SMILES: CCOC(=O)C1(c2ccnc(NS(=O)(=O)C3CC3)n2)CCN(S(C)(=O)=O)CC1] | Method R using INTC205, [HPLC acidic], 433 (1.63). | No data collected | Cs₂CO₃, NMP |
| INTC216 | 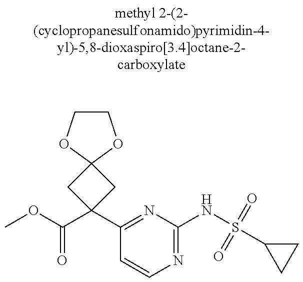 [SMILES: COC(=O)C1(c2ccnc(NS(=O)(=O)C3CC3)n2)CC2(C1)OCCO2] | Method R using INTC208, [UPLC acidic], 370 (0.48). | 11.32 (s, 1H), 8.60 (d, J = 5.2 Hz, 1H), 7.20 (d, J = 5.2 Hz, 1H), 3.87-3.82 (m, 2H), 3.82-3.78 (m, 2H), 3.66 (s, 3H), 3.27- 3.18 (m, 1H), 3.05-2.97 (m, 2H), 2.98-2.84 (m, 2H), 1.17-1.08 (m, 2H), 1.07-0.99 (m, 2H). | Cs₂CO₃, NMP |
| INTC218 | 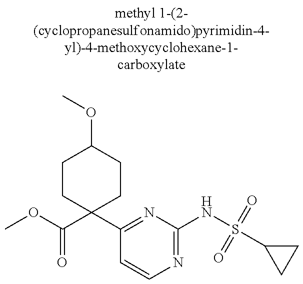 [SMILES: COC(=O)C1(c2ccnc(NS(=O)(=O)C3CC3)n2)CCC(OC)CC1] | Method R using INTC210, [HPLC acidic], 370 (1.62). | 11.25 (s, 1H), 8.57 (d, J = 5.3 Hz, 1H), 7.23-7.11 (m, 1H), 3.65 (s, 3H), 3.24 (s, 3H), 3.23-3.20 (m, 1H), 2.48-2.54 (m 1H, obscured by DMSO peak) 2.37-2.29 (m, 2H), 1.97-1.80 (m, 4H), 1.36- 1.24 (m, 2H), 1.16- 1.01 (m, 4H). | Cs₂CO₃, NMP |
| INTC234 | 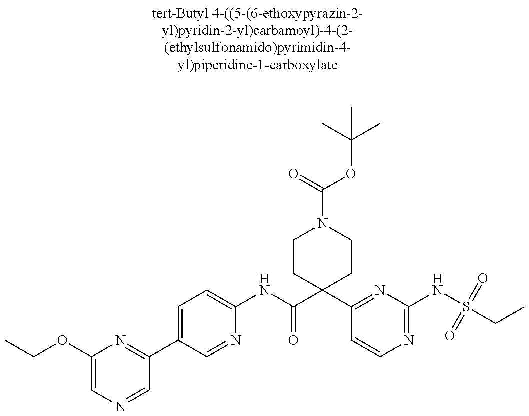 [SMILES: CCOc1cncc(-c2ccc(NC(=O)C3(c4ccnc(NS(=O)(=O)CC)n4)CCN(C(=O)OC(C)(C)C)CC3)nc2)n1] | Method R using INTC233 and ethane sulfonamide, [HPLC Acidic], 613, (2.48) | 11.23 (s, 1H), 10.18 (s, 1H), 9.04 (dd, J = 2.5, 0.8 Hz, 1H), 8.85 (s, 1H), 8.61 (d, J = 5.3 Hz, 1H), 8.51 (dd, J = 8.8, 2.5 Hz, 1H), 8.26 (s, 1H), 8.23- 8.17 (m, 1H), 7.23 (d, J = 5.3 Hz, 1H), 4.48 (q, J = 7.0 Hz, 2H), 3.68-3.62 (m, 2H), 3.56-3.48 (m, 2H), 2.44 (d, J = 14.1 Hz, 2H), 2.13-2.04 (m, 2H), 1.41 (d, J = 4.6 Hz, 11H), 1.13 (t, J = 7.3 Hz, 3H), 0.89-0.79 (m, 3H). | Cs₂CO₃, NMP |
| INTC238 | 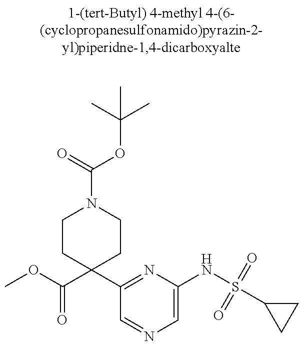 [SMILES: COC(=O)C1(c2cncc(NS(=O)(=O)C3CC3)n2)CCN(C(=O)OC(C)(C)C)CC1] | Method R using INTC237 and cyclopropyl sulfonamide, [UPLC Basic], (M − Boc) + H 341, (1.12) | No NMR recorded | Cs₂CO₃, NMP |
| INTC243 | 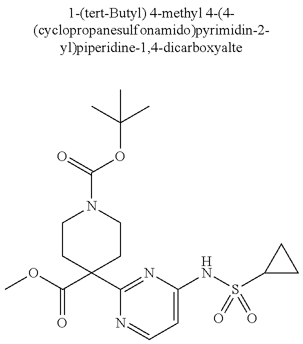 [SMILES: COC(=O)C1(c2nccc(NS(=O)(=O)C3CC3)n2)CCN(C(=O)OC(C)(C)C)CC1] | Method R using INTC242 and cyclopropyl sulfonamide, [HPLC Acidic], (M − Boc) + H 341, (2.03) | 11.31 (s, 1H), 8.51 (d, J = 5.7 Hz, 1H), 6.82 (d, J = 5.7 Hz, 1H), 3.66-3.59 (m, 4H), 3.15-3.06 (m, 4H), 2.18-2.05 (m, 4H), 1.40 (s, 10H), 1.15-1.05 (m, 4H). | Cs2CO₃, NMP |

### N-(5-(6-ethoxypyrazin-2-yl)pyridin-2-yl)-4-(2-(ethylsulfonamido)pyrimidin-4-yl)piperidine-4-carboxamide hydrochloride INTC235

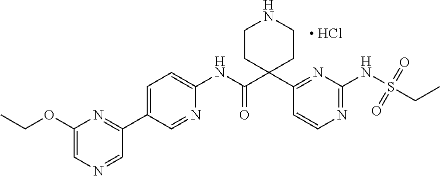 [SMILES: CCOc1cncc(-c2ccc(NC(=O)C3(c4ccnc(NS(=O)(=O)CC)n4)CCNCC3)nc2)n1 ; "· HCl"]

This compound was prepared by Boc-deprotection with HCl of INTC234, [HPLC acidic], 513, (2.72).

### 4-(6-(Cydopropanesulfonamido) pyrazin-2-yl)-N-(5-(6-ethoxypyrazin-2-yl)pyridin-2-yl)piperidine-4-carboxamide hydrochloride INTC240

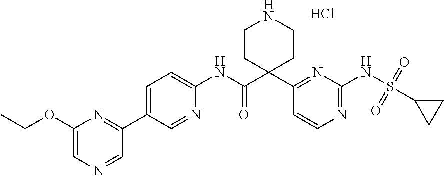 [SMILES: Cl ; CCOc1cncc(-c2ccc(NC(=O)C3(c4ccnc(NS(=O)(=O)C5CC5)n4)CCNCC3)nc2)n1]

This compound was prepared by Boc-deprotection with HCl of INTC239, [UPLC Acidic], 525, (0.83); ¹H NMR (DMSO-d6) 11.18 (s, 1H), 10.39 (s, 1H), 9.02 (d, J=2.4 Hz, 1H), 8.94-8.73 (m, 3H), 8.52 (dd, J=8.8, 2.4 Hz, 1H), 8.41 (s, 1H), 8.26 (s, 1H), 8.23 (s, 1H), 8.19 (d, J=8.8 Hz, 1H), 4.47 (q, J=7.0 Hz, 2H), 3.37-3.22 (m, 2H), 3.15-3.11 (m, 3H), 2.74-2.68 (m, 2H), 2.43-2.40 (m, 2H), 1.39 (t, J=7.0 Hz, 3H), 1.09-1.03 (m, 2H), 0.91-0.82 (m, 2H).

### 4-(4-(Cyclopropanesulfonamido)pyrimidin-2-yl)-N-(5-(6-cyclopropylpyrazin-2-yl)pyridin-2-yl)piperidine-4-carboxamide hydrochloride INTC245

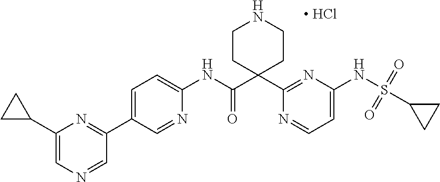 [SMILES: "· HCl" ; O=C(Nc1ccc(-c2cncc(C3CC3)n2)cn1)C1(c2nccc(NS(=O)(=O)C3CC3)n2)CCNCC1]

This compound was prepared by Boc-deprotection with HCl of INTC244, [HPLC Acidic], 521, (1.30); ¹H NMR (DMSO-d6) 11.42 (s, 1H), 10.23 (s, 1H), 9.03-8.96 (m, 2H), 8.89-8.70 (m, 2H), 8.63-8.57 (m, 2H), 8.52-8.48 (m, 1H), 8.21 (d, J=8.8 Hz, 1H), 6.89 (d, J=5.7 Hz, 1H), 3.29-3.07 (m, 5H), 2.53 (s, 2H), 2.29-2.22 (m, 1H), 1.13-1.06 (m, 6H), 0.95-0.89 (m, 2H). 2H not observed potentially obscured broad H₂O peak.

### 4-(4-(Cyclopropanesulfonamido)pyrimidin-2-yl)-N-(5-(6-ethoxypyrazin-2-yl)pyridin-2-yl)piperidine-4-carboxamide hydrochloride INTC247

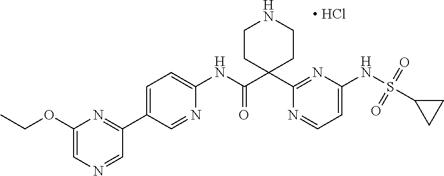 [SMILES: "· HCl" ; CCOc1cncc(-c2ccc(NC(=O)C3(c4nccc(NS(=O)(=O)C5CC5)n4)CCNCC3)nc2)n1]

This compound was prepared by Boc-deprotection with HCl of INTC246, [HPLC Acidic], 521, (1.30).

Method 13: t-BuOK Mediated Amide Coupling from Ester

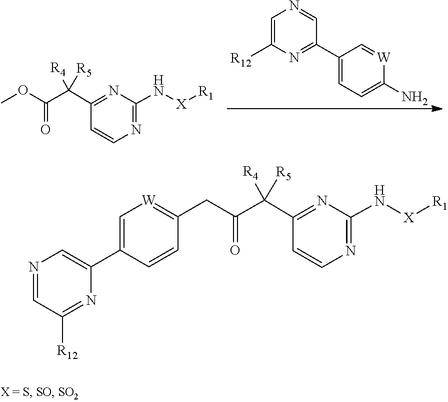

A stirred solution of ester (1.0 eq) and amine (1.1 eq) in THF (10-50 volumes) and DMSO (5 volumes) was cooled to 0° C. under an inert atmosphere. To the reaction mixture was added a solution of f-BuOK (3.3 eq) as a solution THF (10-50 volumes) over 15-80 mins. After addition was complete the reaction was warmed to RT for 1 hr. Reaction was quenched by the addition of aqueous acid either acetic acid or dilute HCl (1 M) until acidic pH was achieved. The reaction was diluted with water and extracted with EtOAc. The organics were combined, dried (phase separator) and concentrated in vacuo. The crude product was purified by reverse or normal phase chromatography or a combination of both.

| | | Synthesis | ¹H NMR Chemical Shift |
|---|---|---|---|
| | Name/Structure | Method, [LCMS | Data |
| | (All examples containing chiral centres | Method], m/z | (DMSO-d6 unless |
| INTC | are racemates unless stated) | (M + H)⁺, (RT/Min) | stated) |
| INTC232 | 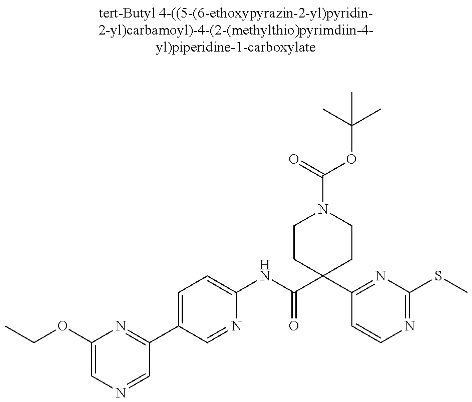 [SMILES: CCOc1cncc(-c2ccc(NC(=O)C3(c4ccnc(SC)n4)CCN(C(=O)OC(C)(C)C)CC3)nc2)n1] | Method 13 using INTC187 and INTD33, [HPLC Acidic], 552, (2.78) | No NMR recorded |

### (4-(6-Ethoxypyrazin-2-yl)-2-fluorophenyl)methanol INTD87

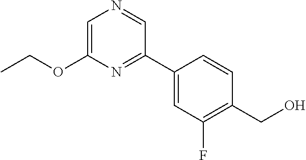 [SMILES: CCOc1cncc(-c2ccc(CO)c(F)c2)n1]

Prepared as for INTD84 using (4-bromo-2-fluorophenyl)methanol (205 mg, 1.00 mmol) and 2-ethoxy-6-(4,4,5,5-tetramethyl-1,3,2-dioxaborolan-2-yl)pyrazine (250 mg, 1.00 mmol) to afford (4-(6-ethoxypyrazin-2-yl)-2-fluorophenyl)methanol (260 mg, 0.995 mmol, quantitative yield) was isolated as a yellow gum. Rt 1.25 min (UPLC, acidic); m/z 249 (M+H)⁺(ES⁺); ¹H NMR (500 MHz, DMSO-d6) δ 8.85 (s, 1H), 8.26 (s, 1H), 7.99 (dd, J=8.0, 1.7 Hz, 1H), 7.91 (dd, J=11.6, 1.7 Hz, 1H), 7.66-7.57 (m, 1H), 5.37 (t, J=5.8 Hz, 1H), 4.62 (d, J=5.8 Hz, 2H), 4.49 (q, J=7.0 Hz, 2H), 1.41 (t, J=7.0 Hz, 3H).

### 4-(6-Ethoxypyrazin-2-yl)-2-fluorobenzaldehyde INTD88

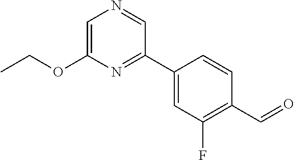 [SMILES: CCOc1cncc(-c2ccc(C=O)c(F)c2)n1]

Prepared as for INTD85 using (4-(6-ethoxypyrazin-2-yl)-2-fluorophenyl)methanol INTD87 (1.00 g, 4.03 mmol) to afford 4-(6-ethoxypyrazin-2-yl)-2-fluorobenzaldehyde (448 mg, 1.78 mmol, 44% yield) as a colourless solid. Rt 0.67 min (UPLC 2, acidic); m/z 247 (M+H)⁺(ES⁺); ¹H NMR (500 MHz, DMSO-d6) δ 10.28 (s, 1H), 8.98 (s, 1H), 8.37 (s, 1H), 8.22-8.13 (m, 2H), 7.99 (dd, J=8.3, 7.3 Hz, 1H), 4.52 (q, J=7.0 Hz, 2H), 1.42 (t, J=7.0 Hz, 3H).

Compounds of Formula (I-a)—Examples

The synthesis of a number of known CTPS1 inhibitors is disclosed in WO2019/179652, WO2019/180244 and WO2020/083975 (see compounds P1 to P225). Such compounds are made using general methods disclosed herein and represent further examples of compounds which are CTPS1 inhibitors. The full synthetic methods and characterising data for compounds P1 to P225 are provided in WO2019/179652, WO2019/180244 and WO2020/083975, each of which is herein incorporated in its entirety by reference.

Amide Formation

Method 1: Amide Coupling Using HATU

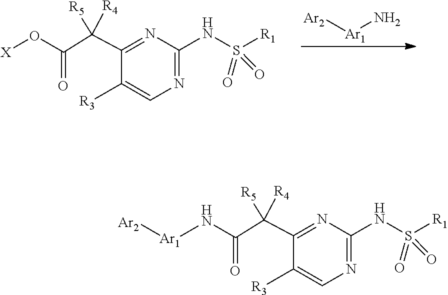

To a stirred suspension of the acid or the potassium salt (1 eq, X=H or K) and DIPEA (6 eq) in DMF (15 vol) the aniline (1 eq) and HATU (1.5 eq) were added. The reaction was stirred at RT for 18 hrs then concentrated in vacuo. MeOH and 2M NaOH (aq) were added. The mixture was stirred for 30 min then concentrated in vacuo. The aqueous phase acidified to pH 6 with 1M HCl (aq) and the product extracted into DCM. The organics were combined, dried (phase separator) and concentrated in vacuo.

The crude product was purified by reverse or normal phase chromatography or a combination of both.

### N-(4-(5-Chloropyridin-3-yl)phenyl)-2-(2-(cyclopropanesulfonamido)pyrimidin-4-yl)butanamide P1

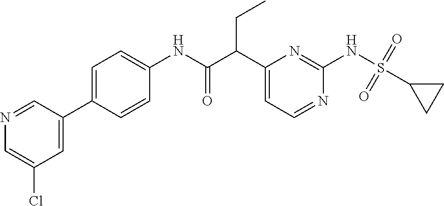 [SMILES: CCC(C(=O)Nc1ccc(-c2cncc(Cl)c2)cc1)c1ccnc(NS(=O)(=O)C2CC2)n1]

Method 2: AlMe₃ Mediated Amide Coupling from Ester

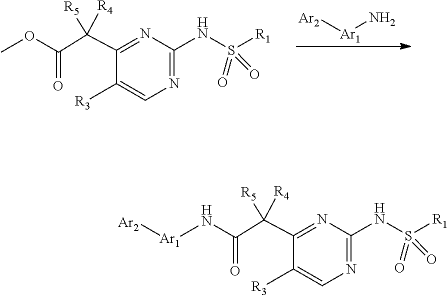

To an ice cooled solution of aniline (2 eq) in toluene (40 volumes) was added AlMe₃ (2.0 M in heptane, 2 eq). The mixture was stirred at this temperature for 5 mins then at RT for 10 mins. To this solution was added ester (1 eq) in one portion and the resultant mixture heated and stirred at 80° C. for 2 hrs. The reaction mixture was cooled in an ice bath and carefully quenched with MeOH (10 volumes). After stirring for 20 mins the mixture was diluted in a mixture of DCM/MeOH (10 volumes), filtered through celite and the filtrate concentrated. The crude product was purified by reverse or normal phase chromatography.

### 1-(2-(Cyclopropanesulfonamido)pyrimidin-4-yl)-N-(4-(6-ethoxypyrazin-2-yl)phenyl)cyclopentanecarboxamide P2

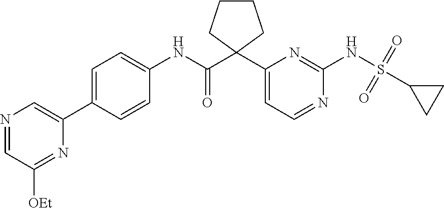 [SMILES: CCOc1cncc(-c2ccc(NC(=O)C3(c4ccnc(NS(=O)(=O)C5CC5)n4)CCCC3)cc2)n1]

### 2-(2-(cyclopropanesulfonamido)pyrimidin-4-yl)-N-(4-(6-methoxypyrazin-2-yl)phenyl)-2-methylpropanamide P3

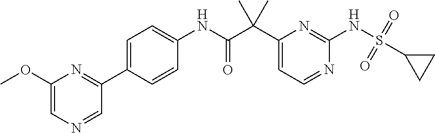 [SMILES: COc1cncc(-c2ccc(NC(=O)C(C)(C)c3ccnc(NS(=O)(=O)C4CC4)n3)cc2)n1]

### 2-(2-(Cyclopropanesulfonamido)pyrimidin-4-yl)-2-methyl-N-(4-(5-(trifluoromethyl)pyridin-3-yl)phenyl)propanamide P4

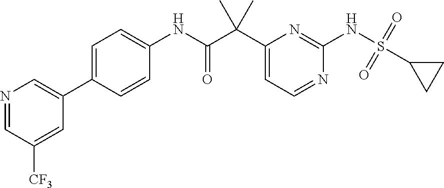 [SMILES: CC(C)(C(=O)Nc1ccc(-c2cncc(C(F)(F)F)c2)cc1)c1ccnc(NS(=O)(=O)C2CC2)n1]

### 2-Methyl-N-(2-methyl-4-(6-methylpyrazin-2-yl)phenyl)-2-(2-(methylsulfonamido)pyrimidin-4-yl)propanamide P5

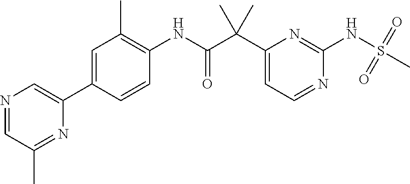 [SMILES: Cc1cncc(-c2ccc(NC(=O)C(C)(C)c3ccnc(NS(C)(=O)=O)n3)c(C)c2)n1]

Method 2b: DABALMe₃ Mediated Amide Coupling from Ester

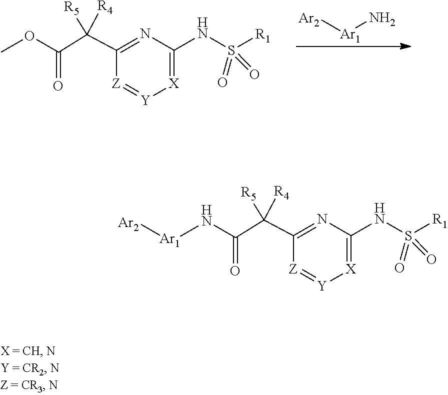

To a solution of ester (1 eq) and aniline (1.5 eq) in toluene (30 volumes) was added DABAL-Me₃ (1.5 eq) and the resulting mixture was heated at 100° C. for 4 h. The reaction mixture was cooled to 0° C. and quenched by careful addition of 1 M HCl (aq, 20 volumes). The aqueous phase was extracted with EtOAc (3×20 volumes). The combined organics were washed with 1 M HCl (aq, 2×10 volumes), dried over Na₂SO₄, filtered and concentrated in vacuo. The crude product was purified by reverse or normal phase chromatography.

Method 3: Amide Coupling from Potassium Salt Using T3P

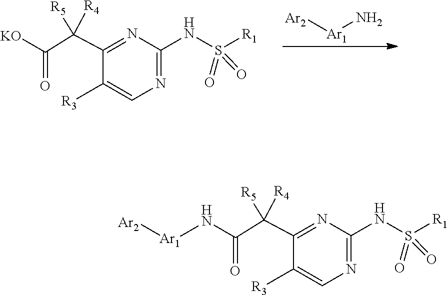

Pyridine (10 eq) followed by T3P (50 wt % in DMF, 2 eq) was added to a stirring solution of amine (1.1 eq) and potassium 2-(2-(cyclopropanesulfonamido)pyrimidin-4-yl)butanoate (1 eq) in DMF (16 volumes). The resulting reaction was stirred at RT for 24 hrs. The crude reaction mixture was concentrated in vacuo then diluted with NH₄Cl (sat. aq) and extracted with DCM. The combined organic extracts were dried (phase separator) and the solvent removed. The crude product was purified by reverse or normal phase chromatography.

### 2-(2-(Cyclopropanesulfonamido)pyrimidin-4-yl)-N-(2-fluoro-4-(pyrazin-2-yl)phenyl)butanamide P6

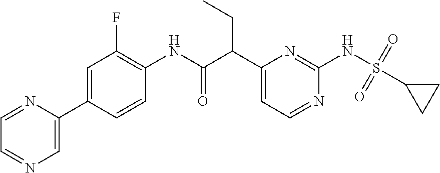 [SMILES: CCC(C(=O)Nc1ccc(-c2cnccn2)cc1F)c1ccnc(NS(=O)(=O)C2CC2)n1]

### 2-(2-(Cyclopropanesulfonamido)pyrimidin-4-yl)-N-(4-(5-(trifluoromethyl)pyridin-3-yl)phenyl)butanamide P7

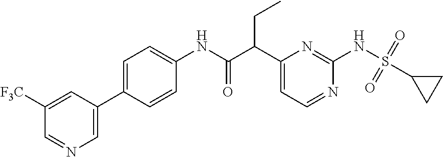 [SMILES: CCC(C(=O)Nc1ccc(-c2cncc(C(F)(F)F)c2)cc1)c1ccnc(NS(=O)(=O)C2CC2)n1]

### 2-(2-(Cyclopropanesulfonamido)pyrimidin-4-yl)-N-(4-(6-(trifluoromethyl)pyrazin-2-yl)phenyl)acetamide P8

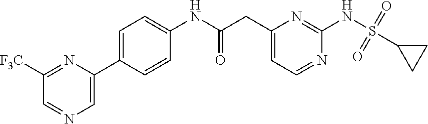 [SMILES: O=C(Cc1ccnc(NS(=O)(=O)C2CC2)n1)Nc1ccc(-c2cncc(C(F)(F)F)n2)cc1]

Method 4: Amide Coupling from Lithium Salt Using T3P

### N-(5-(6-Ethoxypyrazin-2-yl)pyridin-2-yl)-2-fluoro-2-(2-(N-(4-methoxybenzyl)cyclopropanesulfonamido)pyrimidin-4-yl)butanamide INTC51

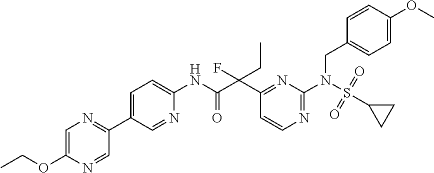 [SMILES: CCOc1cnc(-c2ccc(NC(=O)C(F)(CC)c3ccnc(N(Cc4ccc(OC)cc4)S(=O)(=O)C4CC4)n3)nc2)cn1]

Method 5: NH-Amide Formation Via Amide Deprotection and/or Decarboxylation

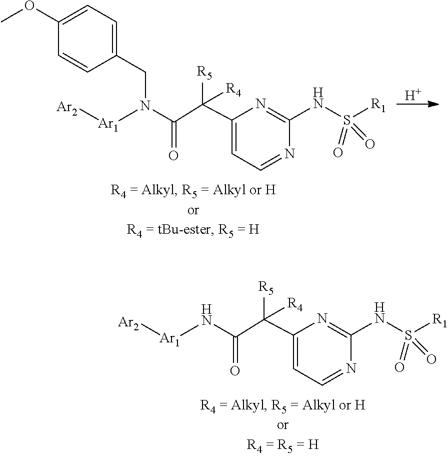

To a solution of the protected amide in DCM a mixture of TFA (88 eq) and triflic acid (1-6 eq) was added and the mixture left stirring at RT for 18-36 hrs and then concentrated in vacuo. The crude product was purified by column chromatography on silica gel or by RP chromatography.

Method 6: Deprotection of Sulfonamide

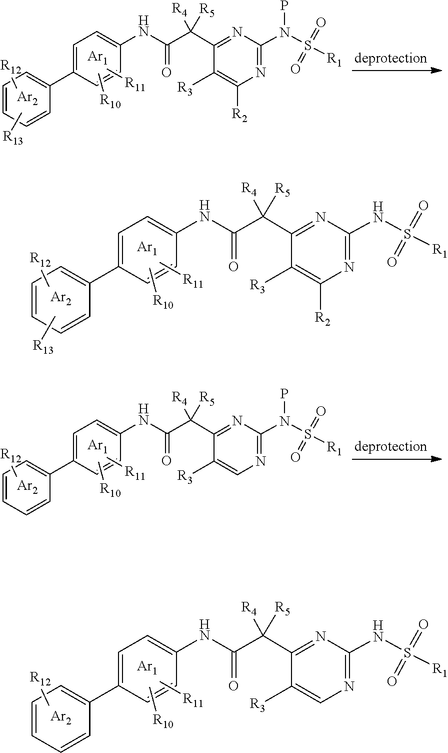

Method 7: Sulfonylation from Aromatic Chloride

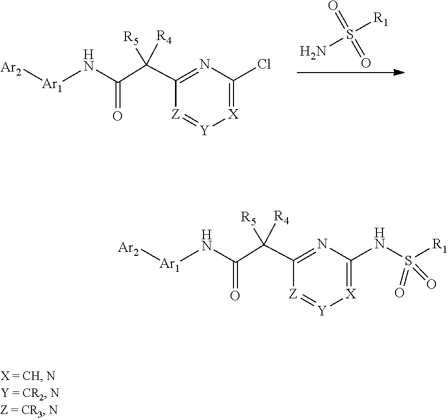

2-Chloro-heteroaromatic intermediate (1 eq), sulfonamide (1.2 eq) and base (2 eq) were dissolved in dioxane (40 volumes). The mixture was degassed (evacuated and backfilled with N₂×3) then catalyst (10 mol %) was added. The resulting mixture was heated under nitrogen at 90° C. for 2 hrs. The mixture was cooled to RT, diluted with sat. NH₄Cl (aq, 80 volumes) and DCM (80 volumes). The phases were separated and the aqueous was extracted with further DCM (2×80 volumes). The combined organics were dried (MgSO₄), filtered and concentrated in vacuo. The crude product was purified by normal phase chromatography or trituration using a suitable solvent.

Method 8: Amide Coupling Using 1-chloro-N,N,2-trimethylprop-1-en-1-amine

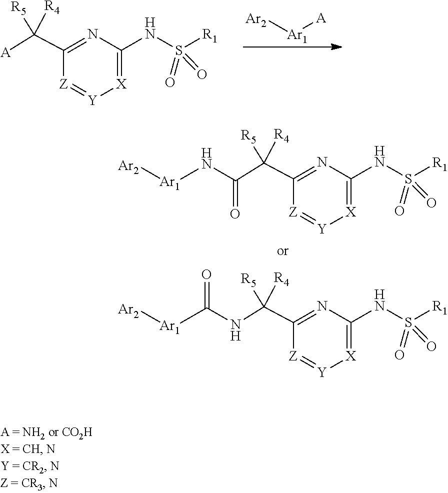

1-Chloro-N,N,2-trimethylprop-1-en-1-amine (2 eq) was added to a solution of carboxylic acid (1 eq) in DCM (20 volumes). The reaction mixture was stirred at RT for 2 hrs. The reaction mixture was concentrated in vacuo and the residue redissolved in DCM (20 volumes) before addition of pyridine (2 mL) followed by addition of the appropriate amine (1.1 eq). The reaction mixture was stirred at RT for 2 hrs. An aqueous work up was performed and the crude product was purified by normal phase chromatography, reverse phase chromatography or trituration from an appropriate solvent.

Method 9: Suzuki ArBr

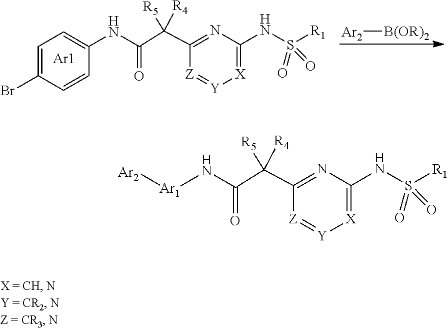

To a suspension of Ar1-Br (1 eq) in dioxane (10 volumes) was added arylboronic acid or ester (1 eq) and a solution of K₂CO₃ (2 eq) in water (5 volumes). The resulting suspension was degassed (N₂, 5 mins). PdCl₂(dppf)-CH₂Cl₂ adduct or other appropriate catalyst (10 mol %) was added and the reaction mixture was stirred at 80° C. for 2 hrs. The reaction mixture was then cooled to RT. An aqueous work up was performed and the crude product was purified by normal phase chromatography, reverse phase chromatography or trituration from an appropriate solvent.

Method 10: T3P with Free Acid

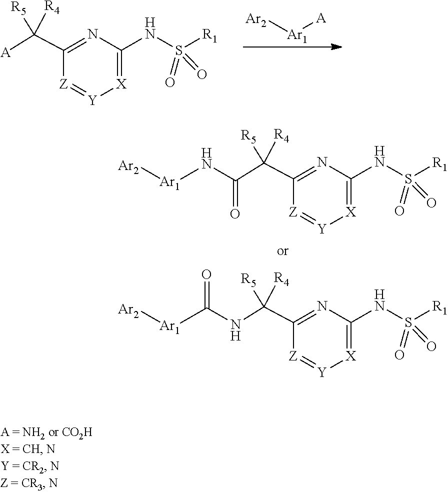

Pyridine (10 eq) followed by T3P (50 wt % in DMF, 2 eq) was added to a stirring solution of amine (1.1 eq) and carboxylic acid (1 eq) in DMF (16 volumes). The resulting reaction was stirred at RT for 24 hrs. The crude reaction mixture was concentrated in vacuo then diluted with NH₄Cl (sat. aq) and extracted with DCM. The combined organic extracts were dried (phase separator) and the solvent removed. The crude product was purified by reverse or normal phase chromatography.

TABLE 4
Compounds P9-P115, P117-P225

| | Name/Structure |
|---|---|
| | (All examples containing chiral |
| | centres are racemates unless |
| P | stated) |
| P9 | 2-(2- |
| | (cyclopropanesulfonamido)pyrimidin- |
| | 4-yl)-N-(5-(6-isopropoxypyrazin-2- |
| | yl)pyridin-2-yl)-2-methylpropanamide |
| | 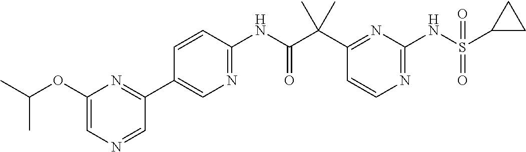 [SMILES: CC(C)Oc1cncc(-c2ccc(NC(=O)C(C)(C)c3ccnc(NS(=O)(=O)C4CC4)n3)nc2)n1] |
| P10 | 2-(2- (cyclopropanesulfonamido)pyrimidin- |
| | 4-yl)-N-(4-(6-ethoxypyrazin-2- |
| | yl)phenyl)-2-ethylbutanamide |
| | 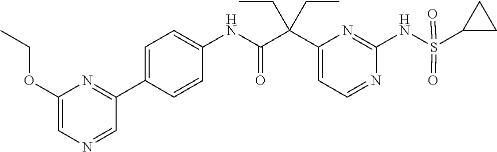 [SMILES: CCOc1cncc(-c2ccc(NC(=O)C(CC)(CC)c3ccnc(NS(=O)(=O)C4CC4)n3)cc2)n1] |
| P11 | 2-(2- |
| | (cyclopropanesulfonamido)pyrimidin- |
| | 4-yl)-N-(2-fluoro-4-(6- |
| | (trifluoromethyl)pyrazin-2- |
| | yl)phenyl)acetamide |
| | 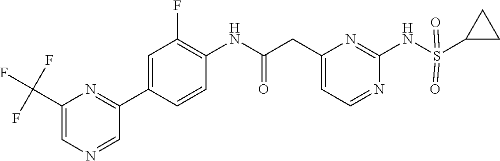 [SMILES: O=C(Cc1ccnc(NS(=O)(=O)C2CC2)n1)Nc1ccc(-c2cncc(C(F)(F)F)n2)cc1F] |
| P12 | 2-(2- |
| | (cyclopropanesulfonamido)pyrimidin- |
| | 4-yl)-N-(2-fluoro-4-(6- |
| | isopropoxypyrazin-2- |
| | yl)phenyl)acetamide |
| | 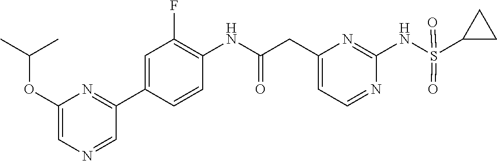 [SMILES: CC(C)Oc1cncc(-c2ccc(NC(=O)Cc3ccnc(NS(=O)(=O)C4CC4)n3)c(F)c2)n1] |
| P13 | 2-(2- |
| | (cyclopropanesulfonamido)pyrimidin- |
| | 4-yl)-N-(4-(5-(trifluoromethyl)pyridin- |
| | 3-yl)phenyl)acetamide |
| | 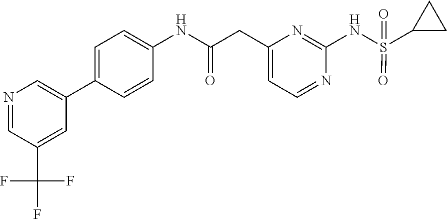 [SMILES: O=C(Cc1ccnc(NS(=O)(=O)C2CC2)n1)Nc1ccc(-c2cncc(C(F)(F)F)c2)cc1] |
| P14 | 2-(2- |
| | (cyclopropanesulfonamido)pyrimidin- |
| | 4-yl)-N-(4-(5-(2,2,2- |
| | trifluoroethoxy)pyridin-3- |
| | yl)phenyl)acetamide |
| | 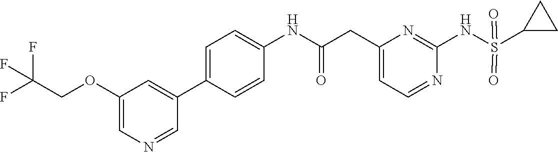 [SMILES: O=C(Cc1ccnc(NS(=O)(=O)C2CC2)n1)Nc1ccc(-c2cncc(OCC(F)(F)F)c2)cc1] |
| P15 | 2-(2-(cyclopropanesulfonamido)-5- |
| | fluoropyrimidin-4-yl)-N-(4-(pyriidn-3- |
| | yl)phenyl)acetamide |
| | 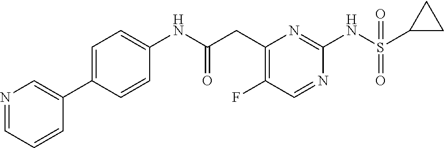 [SMILES: O=C(Cc1nc(NS(=O)(=O)C2CC2)ncc1F)Nc1ccc(-c2cccnc2)cc1] |
| P16 | 2-(2- |
| | (cyclopropanesulfonamido)pyrimidin- |
| | 4-yl)-N-(4-(pyridin-3- |
| | yl)phenyl)acetamide |
| | 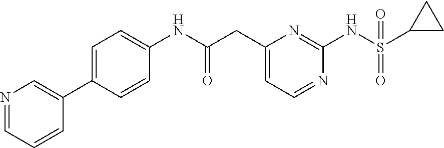 [SMILES: O=C(Cc1ccnc(NS(=O)(=O)C2CC2)n1)Nc1ccc(-c2cccnc2)cc1] |
| P17 | N-([1,1′-biphenyl]-4-yl)-2-(2- |
| | (cyclopropanesulfonamido)pyrimidin- |
| | 4-yl)acetamide |
| | 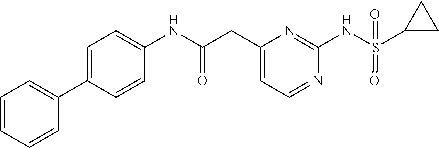 [SMILES: O=C(Cc1ccnc(NS(=O)(=O)C2CC2)n1)Nc1ccc(-c2ccccc2)cc1] |
| P18 | 2-(2- |
| | (cyclopropanesulfonamido)pyrimidin- |
| | 4-yl)-N-(4-(6-ethoxypyrazin-2- |
| | yl)phenyl)acetamide |
| | 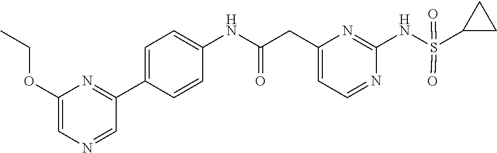 [SMILES: CCOc1cncc(-c2ccc(NC(=O)Cc3ccnc(NS(=O)(=O)C4CC4)n3)cc2)n1] |
| P19 | 2-(2- |
| | (cyclopropanesulfonamido)pyrimidin- |
| | 4-yl)-N-(4-(6-methoxypyrazin-2- |
| | yl)phenyl)acetamide |
| | 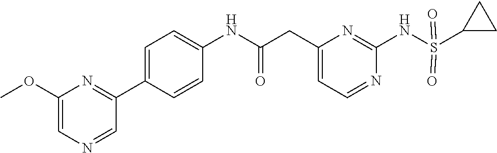 [SMILES: COc1cncc(-c2ccc(NC(=O)Cc3ccnc(NS(=O)(=O)C4CC4)n3)cc2)n1] |
| P20 | 2-(2- |
| | (cyclopropanesulfonamido)pyrimidin- |
| | 4-yl)-N-(4-(6-(2,2,2- |
| | trifluoroethoxy)pyrazin-2- |
| | yl)phenyl)acetamide |
| | 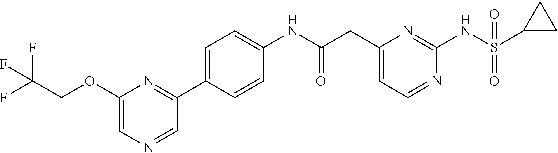 [SMILES: O=C(Cc1ccnc(NS(=O)(=O)C2CC2)n1)Nc1ccc(-c2cncc(OCC(F)(F)F)n2)cc1] |
| P21 | 2-(2- |
| | (cyclopropanesulfonamido)pyrimidin- |
| | 4-yl)-N-(4-(6-isopropoxypyrazin-2- |
| | yl)phenyl)acetamide |
| | 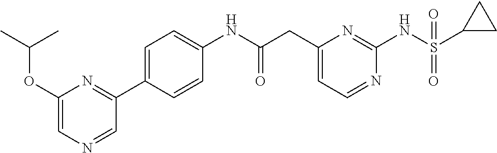 [SMILES: CC(C)Oc1cncc(-c2ccc(NC(=O)Cc3ccnc(NS(=O)(=O)C4CC4)n3)cc2)n1] |
| P22 | 2-(2- |
| | (cyclobutanesulfonamido)pyrimidin- |
| | 4-yl)-N-(5-(6-ethoxypyrazin-2- |
| | yl)pyridin-2-yl)-2-methylpropanamide |
| | 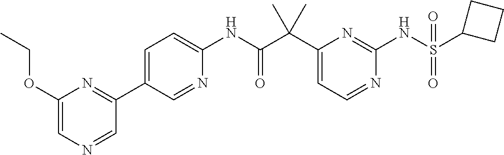 [SMILES: CCOc1cncc(-c2ccc(NC(=O)C(C)(C)c3ccnc(NS(=O)(=O)C4CCC4)n3)nc2)n1] |
| P23 | 2-(2- |
| | (cyclobutanesulfonamido)pyrimidin- |
| | 4-yl)-N-(2-fluoro-4-(6- |
| | isopropoxypyrazin-2-yl)phenyl)-2- |
| | methylpropanamide |
| | 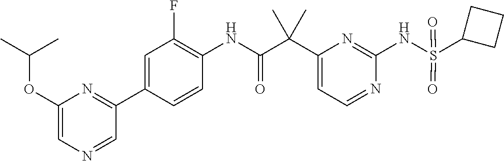 [SMILES: CC(C)Oc1cncc(-c2ccc(NC(=O)C(C)(C)c3ccnc(NS(=O)(=O)C4CCC4)n3)c(F)c2)n1] |
| P24 | 2-(2- |
| | (cyclobutanesulfonamido)pyrimidin- |
| | 4-yl)-N-(4-(6-ethoxypyrazin-2-yl)-2- |
| | methylphenyl)-2-methylpropanamide |
| | 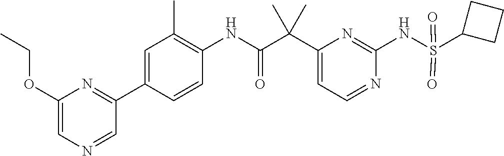 [SMILES: CCOc1cncc(-c2ccc(NC(=O)C(C)(C)c3ccnc(NS(=O)(=O)C4CCC4)n3)c(C)c2)n1] |
| P25 | 2-(2- |
| | (cyclobutanesulfonamido)pyrimidin- |
| | 4-yl)-N-(4-(6-methoxypyrazin-2- |
| | yl)phenyl)-2-methylpropanamide |
| | 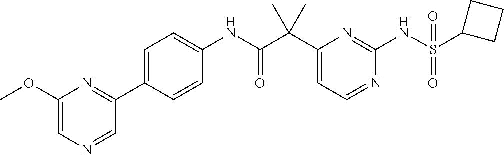 [SMILES: COc1cncc(-c2ccc(NC(=O)C(C)(C)c3ccnc(NS(=O)(=O)C4CCC4)n3)cc2)n1] |
| P26 | 2-(2- |
| | (cyclobutanesulfonamido)pyrimidin- |
| | 4-yl)-N-(4-(6-ethoxypyrazin-2- |
| | yl)phenyl)-2-methylpropanamide |
| | 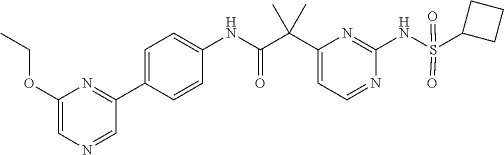 [SMILES: CCOc1cncc(-c2ccc(NC(=O)C(C)(C)c3ccnc(NS(=O)(=O)C4CCC4)n3)cc2)n1] |
| P27 | 2-(2- |
| | (cyclopropanesulfonamido)pyrimidin- |
| | 4-yl)-N-(5-(6-ethoxypyrazin-2-yl)-3- |
| | fluoropyridin-2-yl)-2- |
| | methylpropanamide |
| | 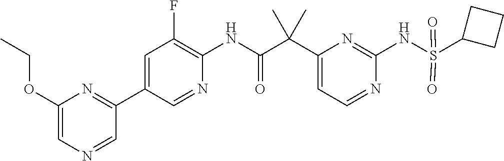 [SMILES: CCOc1cncc(-c2cnc(NC(=O)C(C)(C)c3ccnc(NS(=O)(=O)C4CCC4)n3)c(F)c2)n1] |
| P28 | 2-(2- |
| | (cyclopropanesulfonamido)pyrimidin- |
| | 4-yl)-N-(5′-ethoxy-[3,3′-bipyridin]-6- |
| | yl)-2-methylpropanamide |
| | 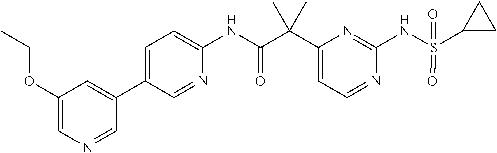 [SMILES: CCOc1cncc(-c2ccc(NC(=O)C(C)(C)c3ccnc(NS(=O)(=O)C4CC4)n3)nc2)c1] |
| P29 | N-([3,3′-bipyridin]-6-yl)-2-(2- |
| | (cyclopropanesulfonamido)pyrimidin- |
| | 4-yl)-2-methylpropanamide |
| | 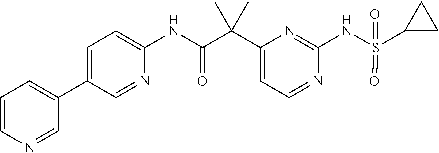 [SMILES: CC(C)(C(=O)Nc1ccc(-c2cccnc2)cn1)c1ccnc(NS(=O)(=O)C2CC2)n1] |
| P30 | 2-(2- |
| | (cyclopropanesulfonamido)pyrimidin- |
| | 4-yl)-2-methyl-N-(5-(6- |
| | (trifluoromethyl)pyrazin-2-yl)pyridin- |
| | 2-yl)propanamide |
| | 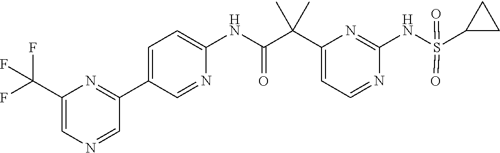 [SMILES: CC(C)(C(=O)Nc1ccc(-c2cncc(C(F)(F)F)n2)cn1)c1ccnc(NS(=O)(=O)C2CC2)n1] |
| P31 | 2-(2- |
| | (cyclopropanesulfonamido)pyrimidin- |
| | 4-yl)-N-(5-(6-ethoxypyrazin-2- |
| | yl)pyridin-2-yl)-2-methylpropanamide |
| | 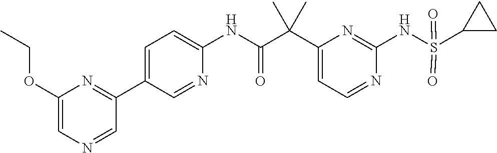 [SMILES: CCOc1cncc(-c2ccc(NC(=O)C(C)(C)c3ccnc(NS(=O)(=O)C4CC4)n3)nc2)n1] |
| P32 | 2-(2- |
| | (cyclopropanesulfonamido)pyrimidin- |
| | 4-yl)-N-(5-(6-cyclopropoxypyrazin-2- |
| | yl)pyridin-2-yl)-2-methylpropanamide |
| | 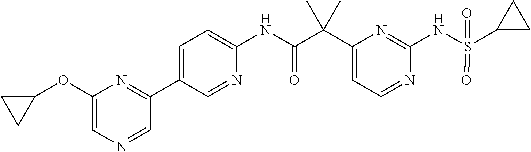 [SMILES: CC(C)(C(=O)Nc1ccc(-c2cncc(OC3CC3)n2)cn1)c1ccnc(NS(=O)(=O)C2CC2)n1] |
| P33 | N-(2-chloro-4-(6-ethoxypyrazin-2- |
| | yl)phenyl)-2-(2- |
| | (cyclopropanesulfonamido)pyrimidin- |
| | 4-yl)-2-methylpropanamide |
| | 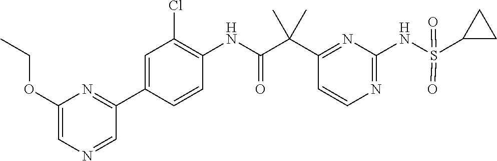 [SMILES: CCOc1cncc(-c2ccc(NC(=O)C(C)(C)c3ccnc(NS(=O)(=O)C4CC4)n3)c(Cl)c2)n1] |
| P34 | N-(2-cyano-4-(6-ethoxypyrazin-2- |
| | yl)phenyl)-2-(2- |
| | (cyclopropanesulfonamido)pyrimidin- |
| | 4-yl)-2-methylpropanamide |
| | 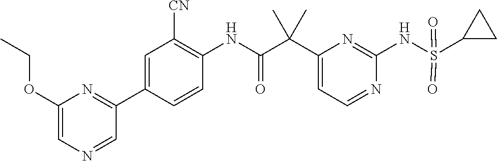 [SMILES: CCOc1cncc(-c2ccc(NC(=O)C(C)(C)c3ccnc(NS(=O)(=O)C4CC4)n3)c(C#N)c2)n1] |
| P35 | 2-(2- |
| | (cyclopropanesulfonamido)pyrimidin- |
| | 4-yl)-N-(2-fluoro-4-(5- |
| | isopropoxypyridin-3-yl)phenyl)-2- |
| | methylpropanamide |
| | 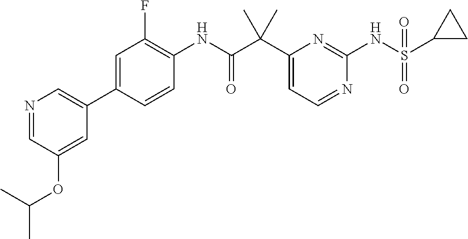 [SMILES: CC(C)Oc1cncc(-c2ccc(NC(=O)C(C)(C)c3ccnc(NS(=O)(=O)C4CC4)n3)c(F)c2)c1] |
| P36 | 2-(2- |
| | (cyclopropanesulfonamido)pyrimidin- |
| | 4-yl)-N-(2-fluoro-4-(pyridin-3- |
| | yl)phenyl)-2-methylpropanamide |
| | 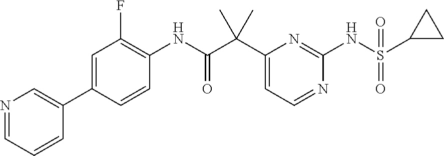 [SMILES: CC(C)(C(=O)Nc1ccc(-c2cccnc2)cc1F)c1ccnc(NS(=O)(=O)C2CC2)n1] |
| P37 | 2-(2- |
| | (cyclopropanesulfonamido)pyrimidin- |
| | 4-yl)-N-(2-fluoro-4-(6- |
| | (trifluoromethyl)pyrazin-2-yl)phenyl)- |
| | 2-methylpropanamide |
| | 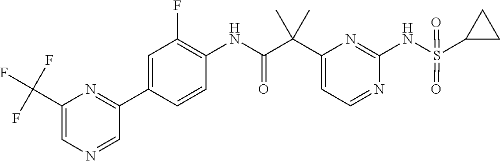 [SMILES: CC(C)(C(=O)Nc1ccc(-c2cncc(C(F)(F)F)n2)cc1F)c1ccnc(NS(=O)(=O)C2CC2)n1] |
| P38 | 2-(2- |
| | (cyclopropanesulfonamido)pyrimidin- |
| | 4-yl)-N-(4-(6-ethoxypyrazin-2-yl)-2- |
| | fluorophenyl)-2-methylpropanamide |
| | 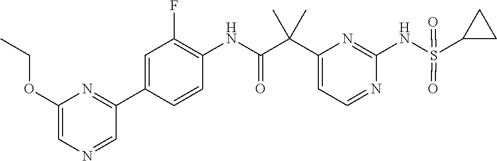 [SMILES: CCOc1cncc(-c2ccc(NC(=O)C(C)(C)c3ccnc(NS(=O)(=O)C4CC4)n3)c(F)c2)n1] |
| P39 | 2-(2- |
| | (cyclopropanesulfonamido)pyrimidin- |
| | 4-yl)-N-(2-fluoro-4-96- |
| | isopropoxypyrazin-2-yl)phenyl)-2- |
| | methylpropanamide |
| | 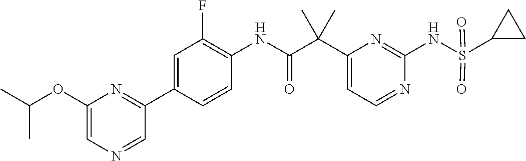 [SMILES: CC(C)Oc1cncc(-c2ccc(NC(=O)C(C)(C)c3ccnc(NS(=O)(=O)C4CC4)n3)c(F)c2)n1] |
| P40 | 2-(2- |
| | (cyclopropanesulfonamido)pyrimidin- |
| | 4-yl)-N-(4-(6-ethoxypyrazin-2-yl)-2- |
| | fluoro-5-methylphenyl)-2- |
| | methylpropanamide |
| | 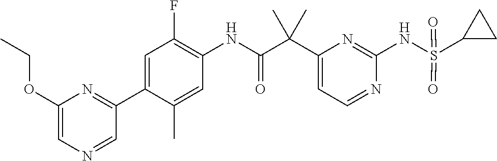 [SMILES: CCOc1cncc(-c2cc(F)c(NC(=O)C(C)(C)c3ccnc(NS(=O)(=O)C4CC4)n3)cc2C)n1] |
| P41 | 2-(2- |
| | (cyclopropanesulfonamido)pyrimidin- |
| | 4-yl)-N-(4-(6-ethoxypyrazin-2-yl)-2,6- |
| | difluorophenyl)-2- |
| | methylpropanamide |
| | 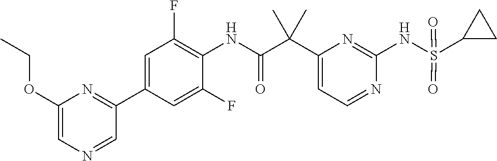 [SMILES: CCOc1cncc(-c2cc(F)c(NC(=O)C(C)(C)c3ccnc(NS(=O)(=O)C4CC4)n3)c(F)c2)n1] |
| P42 | 2-(2- |
| | (cyclopropanesulfonamido)pyrimidin- |
| | 4-yl)-N-(2-fluoro-4-(pyrazin-2- |
| | yl)phenyl)-2-methylpropanamide |
| | 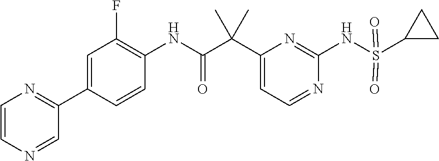 [SMILES: CC(C)(C(=O)Nc1ccc(-c2cnccn2)cc1F)c1ccnc(NS(=O)(=O)C2CC2)n1] |
| P43 | 2-(2- |
| | (cyclopropanesulfonamido)pyrimidin- |
| | 4-yl)-2-methyl-N-(2-methyl-4-(6- |
| | (trifluoromethyl)pyrazin-2- |
| | yl)phenyl)propanamide |
| | 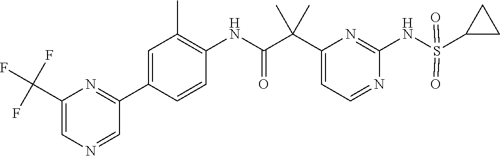 [SMILES: Cc1cc(-c2cncc(C(F)(F)F)n2)ccc1NC(=O)C(C)(C)c1ccnc(NS(=O)(=O)C2CC2)n1] |
| P44 | 2-(2- |
| | (cyclopropanesulfonamido)pyrimidin- |
| | 4-yl)-N-(4-(6-ethoxypyrazin-2-yl)-2,3- |
| | dimethylphenyl)-2- |
| | methylpropanamide |
| | 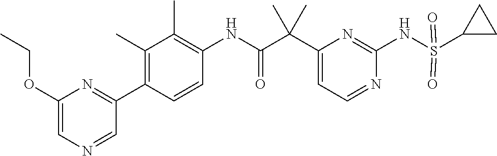 [SMILES: CCOc1cncc(-c2ccc(NC(=O)C(C)(C)c3ccnc(NS(=O)(=O)C4CC4)n3)c(C)c2C)n1] |
| P45 | 2-(2- |
| | (cyclopropanesulfonamido)pyrimidin- |
| | 4-yl)-N-(4-(6-ethoxypyrazin-2-yl)-5- |
| | fluoro-2-methylphenyl)-2- |
| | methylpropanamide |
| | 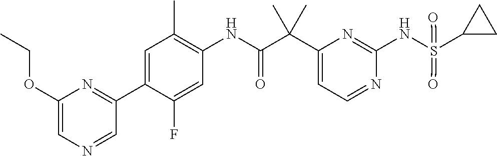 [SMILES: CCOc1cncc(-c2cc(C)c(NC(=O)C(C)(C)c3ccnc(NS(=O)(=O)C4CC4)n3)cc2F)n1] |
| P46 | 2-(2- |
| | (cyclopropanesulfonamido)pyrimidin- |
| | 4-yl)-N-(4-(6-ethoxypyrazin-2-yl)-2,5- |
| | dimethylphenyl)-2- |
| | methylpropanamide |
| | 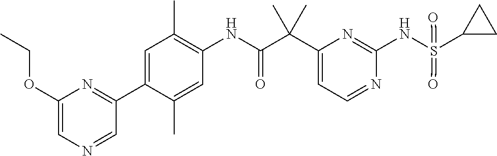 [SMILES: CCOc1cncc(-c2cc(C)c(NC(=O)C(C)(C)c3ccnc(NS(=O)(=O)C4CC4)n3)cc2C)n1] |
| P47 | 2-(2- |
| | (cyclopropanesulfonamido)pyrimidin- |
| | 4-yl)-N-(4-(6-ethoxypyrazin-2-yl)-2- |
| | (trifluoromethoxy)phenyl)-2- |
| | methylpropanamide |
| | 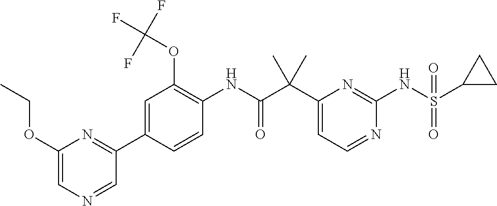 [SMILES: CCOc1cncc(-c2ccc(NC(=O)C(C)(C)c3ccnc(NS(=O)(=O)C4CC4)n3)c(OC(F)(F)F)c2)n1] |
| P48 | 2-(2- |
| | (cyclopropanesulfonamido)pyrimidin- |
| | 4-yl)-N-(4-(6-ethoxypyrazin-2-yl)-5- |
| | fluoro-2-methoxyphenyl)-2- |
| | methylpropanamide |
| | 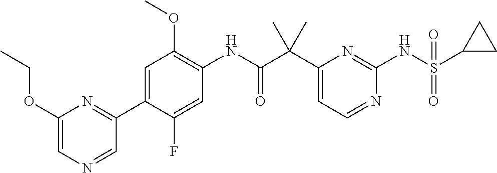 [SMILES: CCOc1cncc(-c2cc(OC)c(NC(=O)C(C)(C)c3ccnc(NS(=O)(=O)C4CC4)n3)cc2F)n1] |
| P49 | 2-(2- |
| | (cyclopropanesulfonamido)pyrimidin- |
| | 4-yl)-N-(4-(6-ethoxypyrazin-2-yl)-2- |
| | methoxyphenyl)-2- |
| | methylpropanamide |
| | 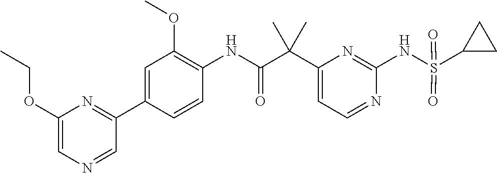 [SMILES: CCOc1cncc(-c2ccc(NC(=O)C(C)(C)c3ccnc(NS(=O)(=O)C4CC4)n3)c(OC)c2)n1] |
| P50 | 2-(2- |
| | (cyclopropanesulfonamido)pyrimidin- |
| | 4-yl)-2-methyl-N-(4-(pyrimidin-5- |
| | yl)phenyl)propanamide |
| | 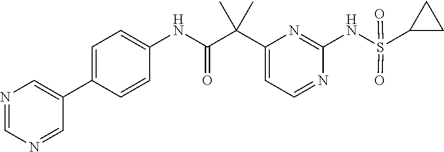 [SMILES: CC(C)(C(=O)Nc1ccc(-c2cncnc2)cc1)c1ccnc(NS(=O)(=O)C2CC2)n1] |
| P51 | N-(4-(-chloropyridin-3-yl)phenyl)-2- |
| | (2- |
| | (cyclopropanesulfonamido)pyrimidin- |
| | 4-yl)-2-methylpropanamide |
| | 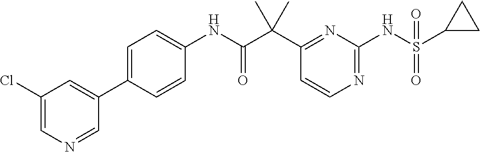 [SMILES: CC(C)(C(=O)Nc1ccc(-c2cncc(Cl)c2)cc1)c1ccnc(NS(=O)(=O)C2CC2)n1] |
| P52 | N-(4-(5-cyanopyridin-3-yl)phenyl)-2- |
| | (2- |
| | (cyclopropanesulfonamido)pyrimidin- |
| | 4-yl)-2-methylpropanamide |
| | 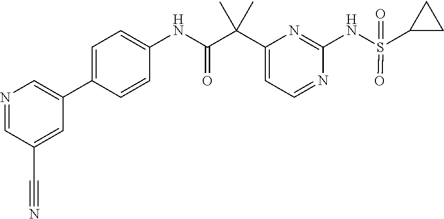 [SMILES: CC(C)(C(=O)Nc1ccc(-c2cncc(C#N)c2)cc1)c1ccnc(NS(=O)(=O)C2CC2)n1] |
| P53 | 2-(2- |
| | (cyclopropanesulfonamido)pyrimidin- |
| | 4-yl)-N-(4-(5-fluoropyridin-3- |
| | yl)phenyl)-2-methylpropanamide |
| | 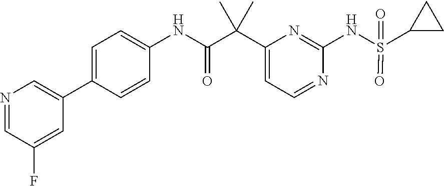 [SMILES: CC(C)(C(=O)Nc1ccc(-c2cncc(F)c2)cc1)c1ccnc(NS(=O)(=O)C2CC2)n1] |
| P54 | 2-(2- |
| | (cyclopropanesulfonamido)pyrimidin- |
| | 4-yl)-2-methyl-N-(4-(5-methylpyridin- |
| | 3-yl)phenyl)propanamide |
| | 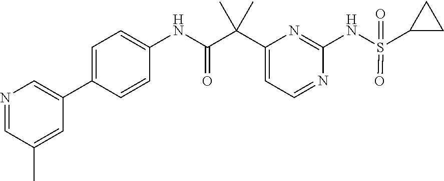 [SMILES: Cc1cncc(-c2ccc(NC(=O)C(C)(C)c3ccnc(NS(=O)(=O)C4CC4)n3)cc2)c1] |
| P55 | 2-(2- |
| | (cyclopropanesulfonamido)pyrimidin- |
| | 4-yl)-N-94-95- |
| | (difluoroethoxy)pyridin-3-yl)phenyl)- |
| | 2-methylpropanamide |
| | 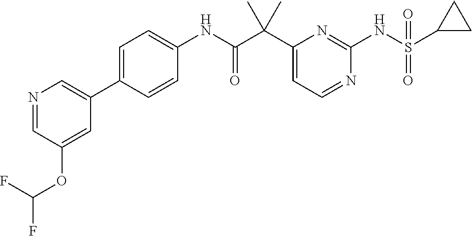 [SMILES: CC(C)(C(=O)Nc1ccc(-c2cncc(OC(F)F)c2)cc1)c1ccnc(NS(=O)(=O)C2CC2)n1] |
| P56 | 2-(2- |
| | (cyclopropanesulfonamido)pyrimidin- |
| | 4-yl)-N-(4-(5-methoxypyridin-3- |
| | yl)phenyl)-2-methylpropanamide |
| | 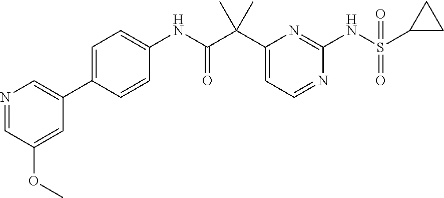 [SMILES: COc1cncc(-c2ccc(NC(=O)C(C)(C)c3ccnc(NS(=O)(=O)C4CC4)n3)cc2)c1] |
| P57 | 2-(2- |
| | (cyclopropanesulfonamido)pyrimidin- |
| | 4-yl)-N-(4-(5-ethoxypyridin-3- |
| | yl)phenyl)-2-methylpropanamide |
| | 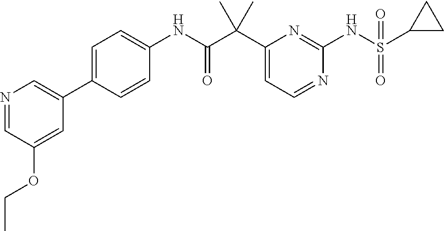 [SMILES: CCOc1cncc(-c2ccc(NC(=O)C(C)(C)c3ccnc(NS(=O)(=O)C4CC4)n3)cc2)c1] |
| P58 | 2-(2- |
| | (cyclopropanesulfonamido)pyrimidin- |
| | 4-yl)-N-(4-(5-isopropoxypyridin-3- |
| | yl)phenyl)-2-methylpropanamide |
| | 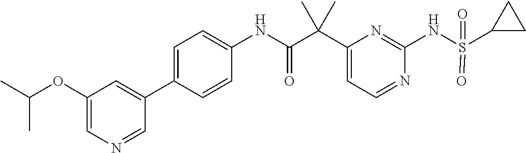 [SMILES: CC(C)Oc1cncc(-c2ccc(NC(=O)C(C)(C)c3ccnc(NS(=O)(=O)C4CC4)n3)cc2)c1] |
| P59 | 2-(2- |
| | (cyclopropanesulfonamido)pyrimidin- |
| | 4-yl)-2-methyl-N-(4-(pyridin-3- |
| | yl)phenyl)propanamide |
| | 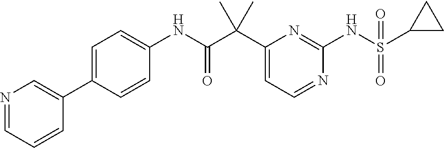 [SMILES: CC(C)(C(=O)Nc1ccc(-c2cccnc2)cc1)c1ccnc(NS(=O)(=O)C2CC2)n1] |
| P60 | 2-(2- |
| | (cyclopropanesulfonamido)pyrimidin- |
| | 4-yl)-2-methyl-N-(3′-(trifluoromethyl)- |
| | [1,1′-biphenyl]-4-yl)propanamide |
| | 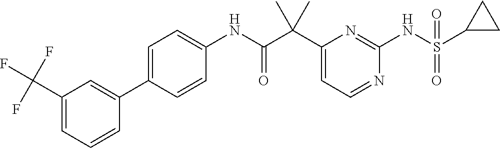 [SMILES: CC(C)(C(=O)Nc1ccc(-c2cccc(C(F)(F)F)c2)cc1)c1ccnc(NS(=O)(=O)C2CC2)n1] |
| P61 | N-(3′-chloro-[1,1′-biphenyl]-4-yl)-2-(2- |
| | (cyclopropanesulfonamido)pyrimidin- |
| | 4-yl)-2-methylpropanamide |
| | 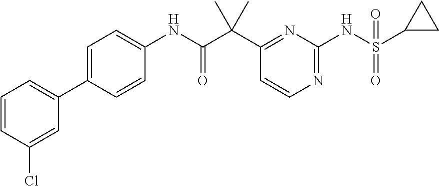 [SMILES: CC(C)(C(=O)Nc1ccc(-c2cccc(Cl)c2)cc1)c1ccnc(NS(=O)(=O)C2CC2)n1] |
| P62 | N-(3′-cyano-[1,1′-biphenyl]-4-yl)-2-(2- |
| | (cyclopropanesulfonamido)pyrimidin- |
| | 4-yl)-2-methylpropanamide |
| | 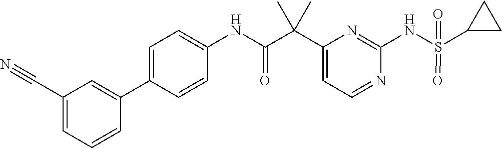 [SMILES: CC(C)(C(=O)Nc1ccc(-c2cccc(C#N)c2)cc1)c1ccnc(NS(=O)(=O)C2CC2)n1] |
| P63 | 2-(2- |
| | (cyclopropanesulfonamido)pyrimidin- |
| | 4-yl)-N-(3′-ethoxy-[1,1′-biphenyl]-4- |
| | yl)-2-methylpropanamide |
| | 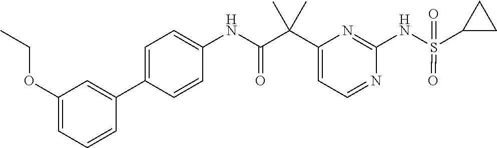 [SMILES: CCOc1cccc(-c2ccc(NC(=O)C(C)(C)c3ccnc(NS(=O)(=O)C4CC4)n3)cc2)c1] |
| P64 | 2-(2- |
| | (cyclopropanesulfonamido)pyrimidin- |
| | 4-yl)-2-methyl-N-(4-(6- |
| | (trifluoromethyl)pyrazin-2- |
| | yl)phenyl)propanamide |
| | 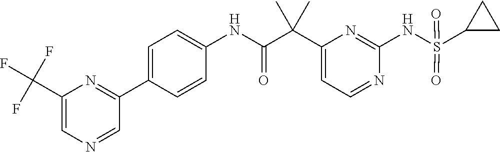 [SMILES: CC(C)(C(=O)Nc1ccc(-c2cncc(C(F)(F)F)n2)cc1)c1ccnc(NS(=O)(=O)C2CC2)n1] |
| P65 | 2-(2- |
| | (cyclopropanesulfonamido)pyrimidin- |
| | 4-yl)-N-(4-(6-ethoxypyrazin-2- |
| | yl)phenyl)-2-methylpropanamide |
| | 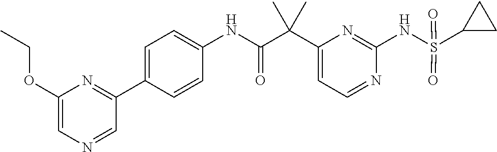 [SMILES: CCOc1cncc(-c2ccc(NC(=O)C(C)(C)c3ccnc(NS(=O)(=O)C4CC4)n3)cc2)n1] |
| P66 | 2-(2- |
| | (cyclopropanesulfonamido)pyrimidin- |
| | 4-yl)-N-(4-(6-cyclopropoxypyrazin-2- |
| | yl)phenyl)-2-methylpropanamide |
| | 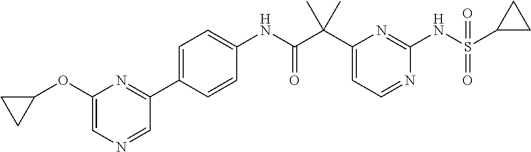 [SMILES: CC(C)(C(=O)Nc1ccc(-c2cncc(OC3CC3)n2)cc1)c1ccnc(NS(=O)(=O)C2CC2)n1] |
| P67 | 2-(2- |
| | (cyclopropanesulfonamido)pyrimidin- |
| | 4-yl)-N-(4-(6-isopropoxypyrazin-2- |
| | yl)phenyl)-2-methylpropanamide |
| | 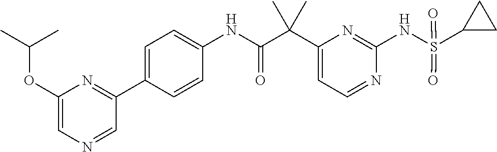 [SMILES: CC(C)Oc1cncc(-c2ccc(NC(=O)C(C)(C)c3ccnc(NS(=O)(=O)C4CC4)n3)cc2)n1] |
| P68 | 2-(2-(cyclopropanesulfonamido)-5- |
| | fluropyrimidin-4-yl)-N-(4-(9- |
| | ethoxypyrazin-2-yl)phenyl)-2- |
| | methylpropanamide |
| | 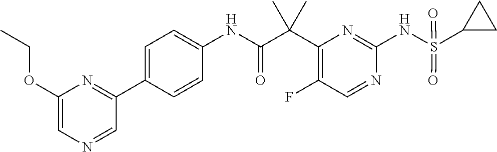 [SMILES: CCOc1cncc(-c2ccc(NC(=O)C(C)(C)c3nc(NS(=O)(=O)C4CC4)ncc3F)cc2)n1] |
| P69 | N-(4-(6-ethoxypyrazin-2-yl)phenyl)-2- |
| | methyl-2-(2-((1-methylcyclopropane)- |
| | 1-sulfonamido)pyrimidin-4- |
| | yl)propanamide |
| | 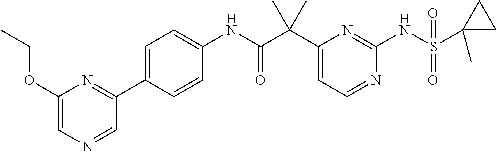 [SMILES: CCOc1cncc(-c2ccc(NC(=O)C(C)(C)c3ccnc(NS(=O)(=O)C4(C)CC4)n3)cc2)n1] |
| P70 | 2-(2-(cyclopropanesulfonamido)-5- |
| | methylpyrimidin-4-yl)-N-(4-(6- |
| | ethoxypyrazin-2-yl)phenyl)-2- |
| | methylpropanamide |
| | 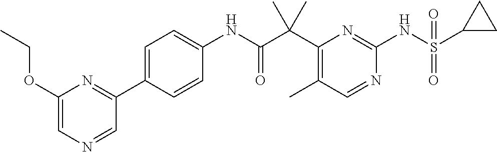 [SMILES: CCOc1cncc(-c2ccc(NC(=O)C(C)(C)c3nc(NS(=O)(=O)C4CC4)ncc3C)cc2)n1] |
| P71 | 2-(2- |
| | (cyclopropanesulfonamido)pyrimidin- |
| | 4-yl)-2-methyl-N-(4-(pyrazin-2- |
| | yl)phenyl)propanamide |
| | 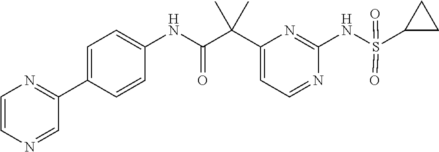 [SMILES: CC(C)(C(=O)Nc1ccc(-c2cnccn2)cc1)c1ccnc(NS(=O)(=O)C2CC2)n1] |
| P72 | N-(4-(6-ethoxypyrazin-2-yl)-2- |
| | fluorophenyl)-2-(2- |
| | (ethylsulfonamido)pyrimidin-4-yl)-2- |
| | methylpropanamide |
| | 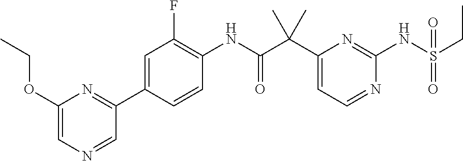 [SMILES: CCOc1cncc(-c2ccc(NC(=O)C(C)(C)c3ccnc(NS(=O)(=O)CC)n3)c(F)c2)n1] |
| P73 | 2-(2-(ethylsulfonamido)pyrimidin-4- |
| | yl)-2-methyl-N-(4-(6- |
| | (trifluoromethyl)pyrazin-2- |
| | yl)phenyl)propanamide |
| | 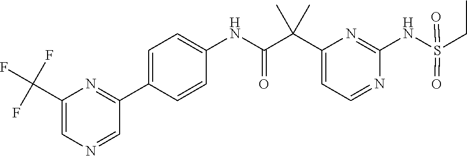 [SMILES: CCS(=O)(=O)Nc1nccc(C(C)(C)C(=O)Nc2ccc(-c3cncc(C(F)(F)F)n3)cc2)n1] |
| P74 | N-(4-(6-ethoxypyrazin-2-yl)phenyl)-2- |
| | (2-(ethylsulfonamido)pyrimidin-4-yl)- |
| | 2-methylpropanamide |
| | 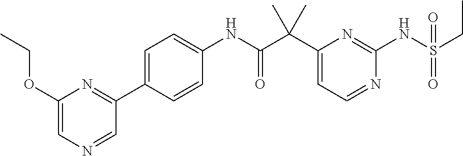 [SMILES: CCOc1cncc(-c2ccc(NC(=O)C(C)(C)c3ccnc(NS(=O)(=O)CC)n3)cc2)n1] |
| P75 | N-(5-(6-ethoxypyrazin-2-yl)-3- |
| | fluoropyridin-2-yl)-2-methyl-2-(2- |
| | (methylsulfonamido)pyrimidin-4- |
| | yl)propanamide |
| | 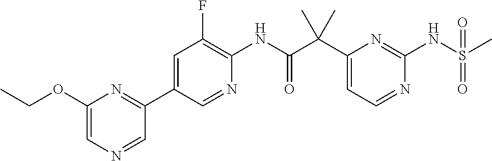 [SMILES: CCOc1cncc(-c2cnc(NC(=O)C(C)(C)c3ccnc(NS(C)(=O)=O)n3)c(F)c2)n1] |
| P76 | N-(5-(6-ethoxypyrazin-2-yl)pyridin-2- |
| | yl)-2-methyl-2-(2- |
| | (methylsulfonamido)pyrimidin-4- |
| | yl)propanamide |
| | 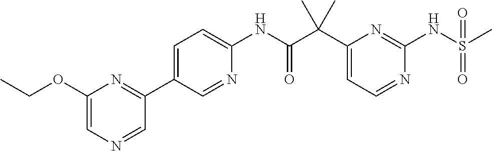 [SMILES: CCOc1cncc(-c2ccc(NC(=O)C(C)(C)c3ccnc(NS(C)(=O)=O)n3)nc2)n1] |
| P77 | N-(2-fluoro-4-(5-isopropoxypyridin-3- |
| | yl)phenyl)-2-methyl-2-(2- |
| | (methylsulfonamido)pyrimidin-4- |
| | yl)propanamide |
| | 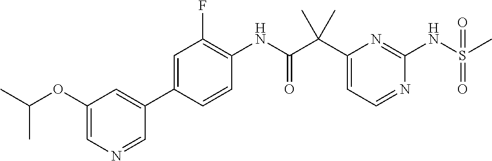 [SMILES: CC(C)Oc1cncc(-c2ccc(NC(=O)C(C)(C)c3ccnc(NS(C)(=O)=O)n3)c(F)c2)c1] |
| P78 | N-(2-fluoro-4-(6-isopropoxypyrazin-2- |
| | yl)phenyl)-2-methyl-2-(2- |
| | (methylsulfonamido)pyrimidin-4- |
| | yl)propanamide |
| | 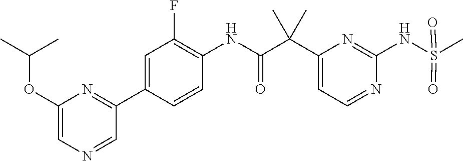 [SMILES: CC(C)Oc1cncc(-c2ccc(NC(=O)C(C)(C)c3ccnc(NS(C)(=O)=O)n3)c(F)c2)n1] |
| P79 | 2-methyl-N-(2-methyl-4-(6- |
| | (trifluoromethyl)pyrazin-2-yl)phenyl)- |
| | 2-(2-(methylsulfonamido)pyrimidin-4- |
| | yl)propanamide |
| | 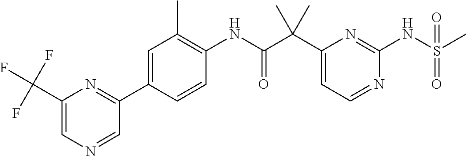 [SMILES: Cc1cc(-c2cncc(C(F)(F)F)n2)ccc1NC(=O)C(C)(C)c1ccnc(NS(C)(=O)=O)n1] |
| P80 | 2-methyl-2-(2- |
| | (methylsulfonamido)pyrimidin-4-yl)- |
| | N-(4-(6-(trifluoromethyl)pyrazin-2- |
| | yl)phenyl)propanamide |
| | 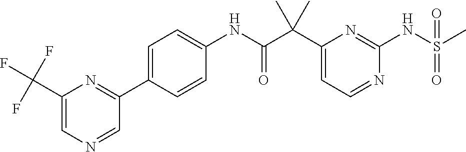 [SMILES: CC(C)(C(=O)Nc1ccc(-c2cncc(C(F)(F)F)n2)cc1)c1ccnc(NS(C)(=O)=O)n1] |
| P81 | N-(4-(6-ethoxypyrazin-2-yl)phenyl)-2- |
| | methyl-2-(2- |
| | (methylsulfonamido)pyrimidin-4- |
| | yl)propanamide |
| | 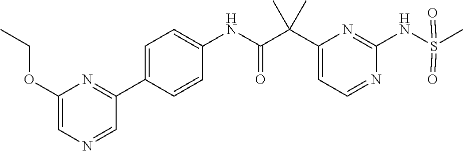 [SMILES: CCOc1cncc(-c2ccc(NC(=O)C(C)(C)c3ccnc(NS(C)(=O)=O)n3)cc2)n1] |
| P82 | 2-(2-((1,1- |
| | dimethylethyl)sulfonamido)pyrimidin- |
| | 4-yl)-N-(4-(6-ethoxypyrazin-2- |
| | yl)phenyl)-2-methylpropanamide |
| | 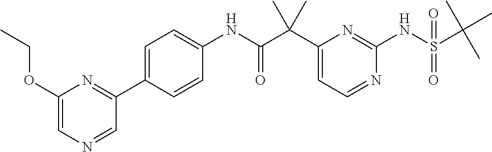 [SMILES: CCOc1cncc(-c2ccc(NC(=O)C(C)(C)c3ccnc(NS(=O)(=O)C(C)(C)C)n3)cc2)n1] |
| P83 | 1-(2- |
| | (cyclopropanesulfonamido)pyrimidin- |
| | 4-yl)-N-(4-(6-ethoxypyrazin-2- |
| | yl)phenyl)cyclopropanecarboxamide |
| | 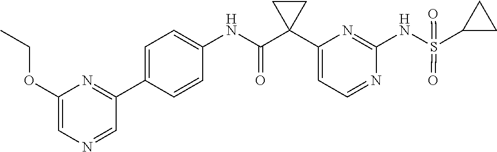 [SMILES: CCOc1cncc(-c2ccc(NC(=O)C3(c4ccnc(NS(=O)(=O)C5CC5)n4)CC3)cc2)n1] |
| P84 | 2-(2- |
| | (cyclopropanesulfonamido)pyrimidin- |
| | 4-yl)-N-(5′-(trifluoromethyl)-[3,3′- |
| | bipyridin]-6-yl)butanamide |
| | 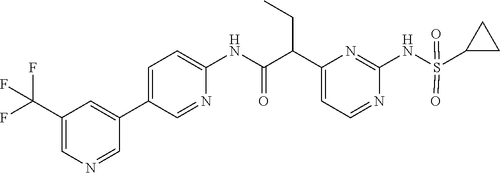 [SMILES: CCC(C(=O)Nc1ccc(-c2cncc(C(F)(F)F)c2)cn1)c1ccnc(NS(=O)(=O)C2CC2)n1] |
| P85 | 2-(2- |
| | (cyclopropanesulfonamido)pyrimidin- |
| | 4-yl)-N-(5′-(2,2,2-trifluoroethoxy)- |
| | [3,3′-bipyridin]-6-yl)butanamide |
| | 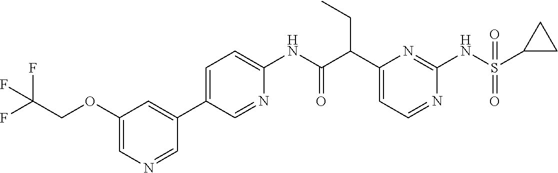 [SMILES: CCC(C(=O)Nc1ccc(-c2cncc(OCC(F)(F)F)c2)cn1)c1ccnc(NS(=O)(=O)C2CC2)n1] |
| P86 | N-([3,3′-bipyridin]-6-yl)-2-(2- |
| | (cyclopropanesulfonamido)pyrimidin- |
| | 4-yl)butanamide |
| | 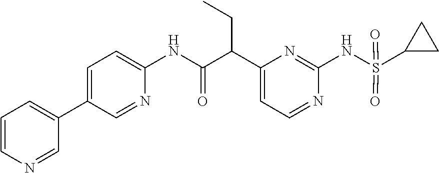 [SMILES: CCC(C(=O)Nc1ccc(-c2cccnc2)cn1)c1ccnc(NS(=O)(=O)C2CC2)n1] |
| P87 | 2-(2- |
| | (cyclopropanesulfonamido)pyrimidin- |
| | 4-yl)-N-(5-(6-(trifluoromethyl)pyrazin- |
| | 2-yl)pyridin-2-yl)butanamide |
| | 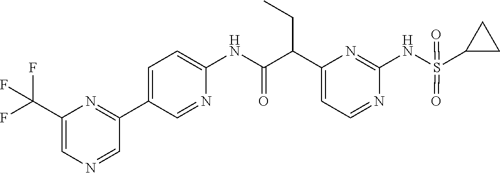 [SMILES: CCC(C(=O)Nc1ccc(-c2cncc(C(F)(F)F)n2)cn1)c1ccnc(NS(=O)(=O)C2CC2)n1] |
| P88 | 2-(2- |
| | (cyclopropanesulfonamido)pyrimidin- |
| | 4-yl)-N-(5-(6-ethoxypyrazin-2- |
| | yl)pyridin-2-yl)butanamide |
| | 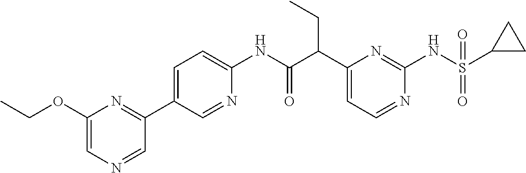 [SMILES: CCOc1cncc(-c2ccc(NC(=O)C(CC)c3ccnc(NS(=O)(=O)C4CC4)n3)nc2)n1] |
| P89 | 2-(2- |
| | (cyclopropanesulfonamido)pyrimidin- |
| | 4-yl)-N-(5-(6-isopropoxypyrazin-2- |
| | yl)pyridin-2-yl)butanamide |
| | 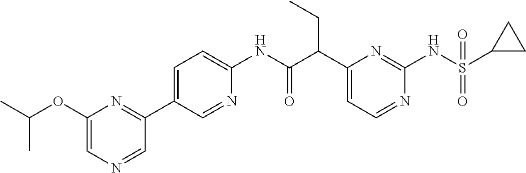 [SMILES: CCC(C(=O)Nc1ccc(-c2cncc(OC(C)C)n2)cn1)c1ccnc(NS(=O)(=O)C2CC2)n1] |
| P90 | N-(4-(5-chloropyridin-3-yl)-2- |
| | fluorophenyl)-2-(2- |
| | (cyclopropanesulfonamido)pyrimidin- |
| | 4-yl)butanamide |
| | 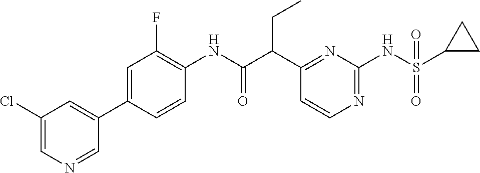 [SMILES: CCC(C(=O)Nc1ccc(-c2cncc(Cl)c2)cc1F)c1ccnc(NS(=O)(=O)C2CC2)n1] |
| P91 | 2-(2- |
| | (cyclopropanesulfonamido)pyrimidin- |
| | 4-yl)-N-(2-fluoro-4-(5-(2,2,2- |
| | trifluoroethoxy)pyridin-3- |
| | yl)phenyl)butanamide |
| | 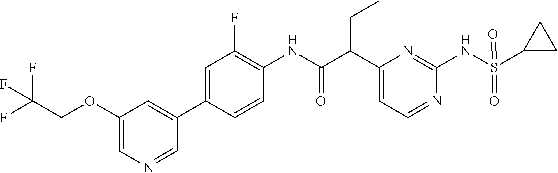 [SMILES: CCC(C(=O)Nc1ccc(-c2cncc(OCC(F)(F)F)c2)cc1F)c1ccnc(NS(=O)(=O)C2CC2)n1] |
| P92 | 2-(2- |
| | (cyclopropanesulfonamido)pyrimidin- |
| | 4-yl)-N-(2-fluoro-4-(5- |
| | isopropoxypyridin-3- |
| | yl)phenyl)butanamide |
| | 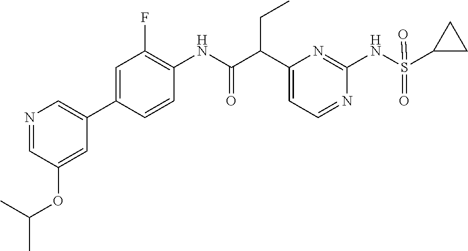 [SMILES: CCC(C(=O)Nc1ccc(-c2cncc(OC(C)C)c2)cc1F)c1ccnc(NS(=O)(=O)C2CC2)n1] |
| P93 | 2-(2- |
| | (cyclopropanesulfonamido)pyrimidin- |
| | 4-yl)-N-(2-fluoro-4-(pyridin-3- |
| | yl)phenyl)butanamide |
| | 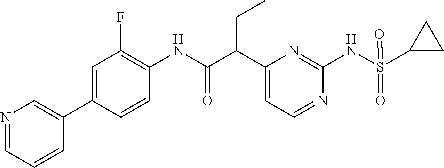 [SMILES: CCC(C(=O)Nc1ccc(-c2cccnc2)cc1F)c1ccnc(NS(=O)(=O)C2CC2)n1] |
| P94 | 2-(2- |
| | (cyclopropanesulfonamido)pyrimidin- |
| | 4-yl)-N-(2-fluoro-4-(6- |
| | (trifluoromethyl)pyrazin-2- |
| | yl)phenyl)butanamide |
| | 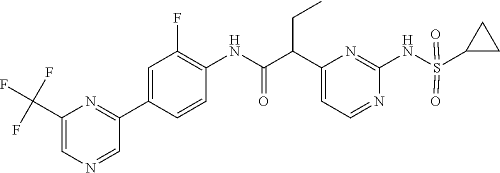 [SMILES: CCC(C(=O)Nc1ccc(-c2cncc(C(F)(F)F)n2)cc1F)c1ccnc(NS(=O)(=O)C2CC2)n1] |
| P95 | 2-(2- |
| | (cyclopropanesulfonamido)pyrimidin- |
| | 4-yl)-N-(2-fluoro-4-(6- |
| | methoxypyrazin-2- |
| | yl)phenyl)butanamide |
| | 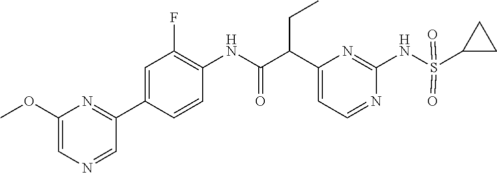 [SMILES: CCC(C(=O)Nc1ccc(-c2cncc(OC)n2)cc1F)c1ccnc(NS(=O)(=O)C2CC2)n1] |
| P96 | 2-(2- |
| | (cyclopropanesulfonamido)pyrimidin- |
| | 4-yl)-N-(4-(6-ethoxypyrazin-2-yl)-2- |
| | fluorophenyl)butanamide |
| | 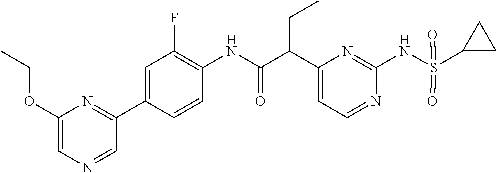 [SMILES: CCOc1cncc(-c2ccc(NC(=O)C(CC)c3ccnc(NS(=O)(=O)C4CC4)n3)c(F)c2)n1] |
| P97 | 2-(2- |
| | (cyclopropanesulfonamido)pyrimidin- |
| | 4-yl)-N-(2-fluoro-4-(6- |
| | isopropoxypyrazin-2- |
| | yl)phenyl)butanamide |
| | 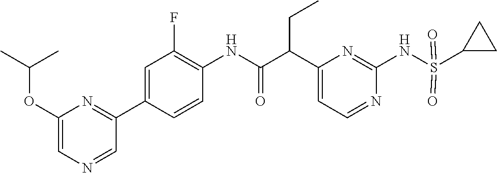 [SMILES: CCC(C(=O)Nc1ccc(-c2cncc(OC(C)C)n2)cc1F)c1ccnc(NS(=O)(=O)C2CC2)n1] |
| P98 | 2-(2- |
| | (cyclopropanesulfonamido)pyrimidin- |
| | 4-yl)-N-(2-fluoro-4-(6-(2,2,2- |
| | trifluoroethoxy)pyrazin-2- |
| | yl)phenyl)butanamide |
| | 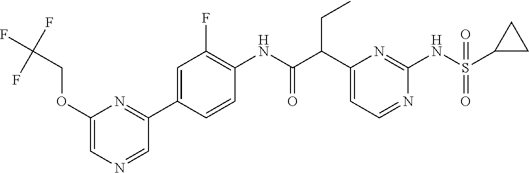 [SMILES: CCC(C(=O)Nc1ccc(-c2cncc(OCC(F)(F)F)n2)cc1F)c1ccnc(NS(=O)(=O)C2CC2)n1] |
| P99 | N-(4-(5-cyanopyridin-3-yl)phenyl)-2- |
| | (2- |
| | (cyclopropanesulfonamido)pyrimidin- |
| | 4-yl)butanamide |
| | 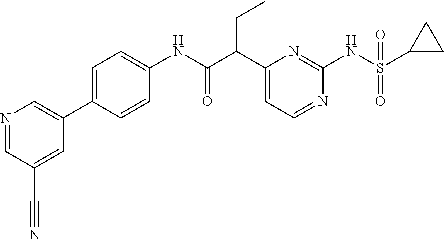 [SMILES: CCC(C(=O)Nc1ccc(-c2cncc(C#N)c2)cc1)c1ccnc(NS(=O)(=O)C2CC2)n1] |
| P100 | 2-(2- |
| | (cyclopropanesulfonamido)pyrimidin- |
| | 4-yl)-N-(4-(5-(2,2,2- |
| | trifluoroethoxy)pyridin-3- |
| | yl)phenyl)butanamide |
| | 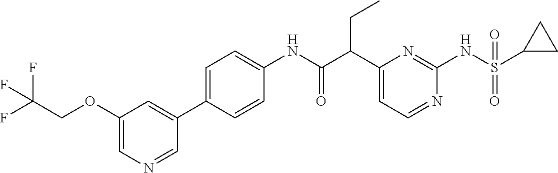 [SMILES: CCC(C(=O)Nc1ccc(-c2cncc(OCC(F)(F)F)c2)cc1)c1ccnc(NS(=O)(=O)C2CC2)n1] |
| P101 | 2-(2- |
| | (cyclopropanesulfonamido)pyrimidin- |
| | 4-yl)-N-(4-(5-isopropoxypyridin-3- |
| | yl)phenyl)butanamide |
| | 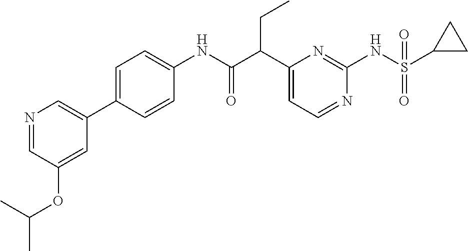 [SMILES: CCC(C(=O)Nc1ccc(-c2cncc(OC(C)C)c2)cc1)c1ccnc(NS(=O)(=O)C2CC2)n1] |
| P102 | 2-(2- |
| | (cyclopropanesulfonamido)pyrimidin- |
| | 4-yl)-N-(4-(pyridin-3- |
| | yl)phenyl)butanamide |
| | 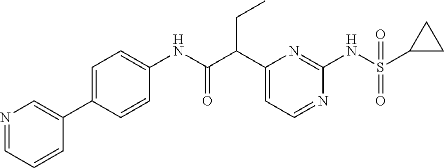 [SMILES: CCC(C(=O)Nc1ccc(-c2cccnc2)cc1)c1ccnc(NS(=O)(=O)C2CC2)n1] |
| P103 | 2-(2- |
| | (cyclopropanesulfonamido)pyrimidin- |
| | 4-yl)-N-(4-(6-(trifluoromethyl)pyrazin- |
| | 2-yl)phenyl)butanamide |
| | 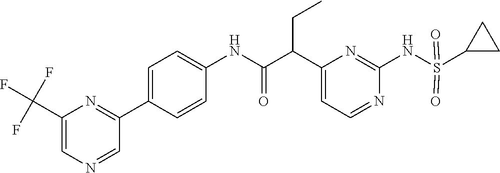 [SMILES: CCC(C(=O)Nc1ccc(-c2cncc(C(F)(F)F)n2)cc1)c1ccnc(NS(=O)(=O)C2CC2)n1] |
| P104 | N-(4-(6-chloropyrazin-2-yl)phenyl)-2- |
| | (2- |
| | (cyclopropanesulfonamido)pyrimidin- |
| | 4-yl)butanamide |
| | 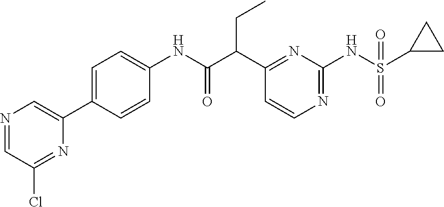 [SMILES: CCC(C(=O)Nc1ccc(-c2cncc(Cl)n2)cc1)c1ccnc(NS(=O)(=O)C2CC2)n1] |
| P105 | 2-(2- |
| | (cyclopropanesulfonamido)pyrimidin- |
| | 4-yl)-N-(4-(6-ethoxypyrazin-2- |
| | yl)phenyl)butanamide |
| | 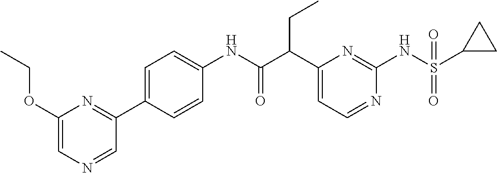 [SMILES: CCOc1cncc(-c2ccc(NC(=O)C(CC)c3ccnc(NS(=O)(=O)C4CC4)n3)cc2)n1] |
| P106 | 2-(2- |
| | (cyclopropanesulfonamido)pyrimidin- |
| | 4-yl)-N-(4-(6-methoxypyrazin-2- |
| | yl)phenyl)butanamide |
| | 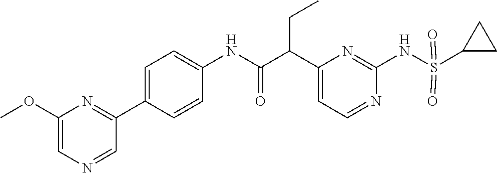 [SMILES: CCC(C(=O)Nc1ccc(-c2cncc(OC)n2)cc1)c1ccnc(NS(=O)(=O)C2CC2)n1] |
| P107 | 2-(2- |
| | (cyclopropanesulfonamido)pyrimidin- |
| | 4-yl)-N-(4-(6-isopropoxypyrazin-2- |
| | yl)phenyl)butanamide |
| | 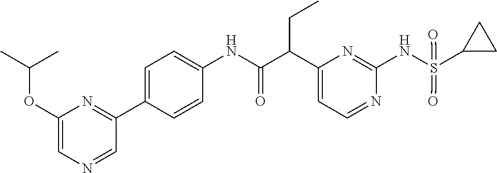 [SMILES: CCC(C(=O)Nc1ccc(-c2cncc(OC(C)C)n2)cc1)c1ccnc(NS(=O)(=O)C2CC2)n1] |
| P108 | 2-(2- |
| | (cyclopropanesulfonamido)pyrimidin- |
| | 4-yl)-N-(4-(9-(2,2,2- |
| | trifluoroethoxy)pyrazin-2- |
| | yl)phenyl)butanamide |
| | 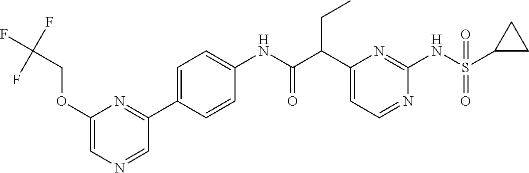 [SMILES: CCC(C(=O)Nc1ccc(-c2cncc(OCC(F)(F)F)n2)cc1)c1ccnc(NS(=O)(=O)C2CC2)n1] |
| P109 | 2-(2- |
| | (cyclopropanesulfonamido)pyrimidin- |
| | 4-yl)-N-(4-(pyrazin-2- |
| | yl)phenyl)butanamide |
| | 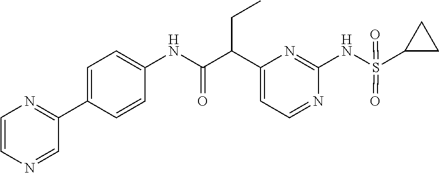 [SMILES: CCC(C(=O)Nc1ccc(-c2cnccn2)cc1)c1ccnc(NS(=O)(=O)C2CC2)n1] |
| P110 | 2-(2- |
| | (cyclopropanesulfonamido)pyrimidin- |
| | 4-yl)-N-(4-(9-ethoxypyrazin-2- |
| | yl)phenyl)-4-methoxypbutanamide |
| | 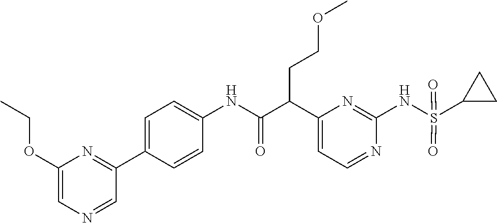 [SMILES: CCOc1cncc(-c2ccc(NC(=O)C(CCOC)c3ccnc(NS(=O)(=O)C4CC4)n3)cc2)n1] |
| P111 | 2-(2- |
| | (cyclopropanesulfonamido)pyrimidin- |
| | 4-yl)-N-(4-(pyridin-3- |
| | yl)phenyl)propanamide |
| | 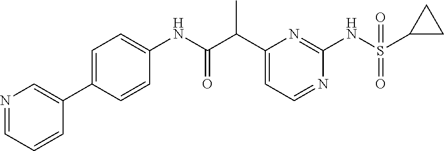 [SMILES: CC(C(=O)Nc1ccc(-c2cccnc2)cc1)c1ccnc(NS(=O)(=O)C2CC2)n1] |
| P112 | 2-(2- |
| | (Cyclopropanesulfonamido)pyrimidin- |
| | 4-yl)-N-(5-(6-ethoxypyrazin-2- |
| | yl)pyridin-2-yl)-2-fluorobutanamide |
| | 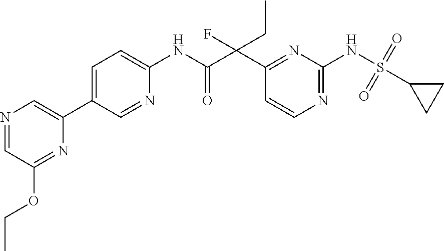 [SMILES: CCOc1cncc(-c2ccc(NC(=O)C(F)(CC)c3ccnc(NS(=O)(=O)C4CC4)n3)nc2)n1] |
| P113 | Single enantiomer - stereochemistry |
| | not assigned 2-(2- |
| | (Cyclopropanesulfonamido)pyrimidin- |
| | 4-yl)-N-(5-(6-ethoxypyrazin-2- |
| | yl)pyridin-2-yl)-2-fluorobutanamide |
| | 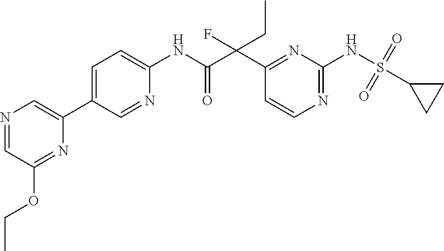 [SMILES: CCOc1cncc(-c2ccc(NC(=O)C(F)(CC)c3ccnc(NS(=O)(=O)C4CC4)n3)nc2)n1] |
| P114 | Single enantiomer - stereochemistry |
| | not assigned 2-(2- |
| | (Cyclopropanesulfonamido)pyrimidin- |
| | 4-yl)-N-(5-(6-ethoxypyrazin-2- |
| | yl)pyridin-2-yl)-2-fluorobutanamide |
| | 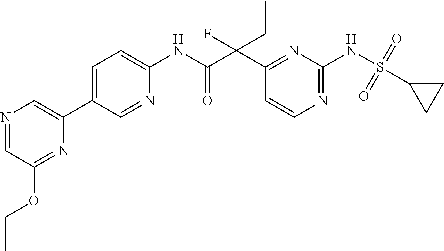 [SMILES: CCOc1cncc(-c2ccc(NC(=O)C(F)(CC)c3ccnc(NS(=O)(=O)C4CC4)n3)nc2)n1] |
| P115 | 4-(2- |
| | (Cyclopropanesulfonamido)pyrimidin- |
| | 4-yl)-N-(5-(6-ethoxypyrazin-2- |
| | yl)pyridin-2-yl)tetrahydro-2H-pyran-4- |
| | carboxamide |
| | 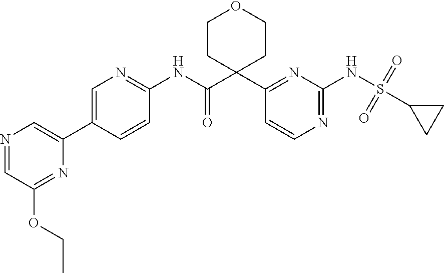 [SMILES: CCOc1cncc(-c2ccc(NC(=O)C3(c4ccnc(NS(=O)(=O)C5CC5)n4)CCOCC3)nc2)n1] |
| P117 | 2-(2- |
| | (cyclopropanesulfonamido)pyrimidin- |
| | 4-yl)-N-(4-(6-ethoxypyrazin-2-yl)-2- |
| | fluorophenyl)-2,2-difluoroacetamide |
| | 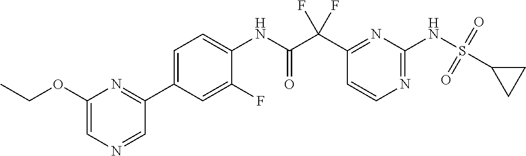 [SMILES: CCOc1cncc(-c2ccc(NC(=O)C(F)(F)c3ccnc(NS(=O)(=O)C4CC4)n3)c(F)c2)n1] |
| P118 | N-((2- |
| | (cyclopropanesulfonamido)pyrimidin-4- |
| | yl)methyl)-4-(6-ethoxypyrazin-2- |
| | yl)benzamide |
| | 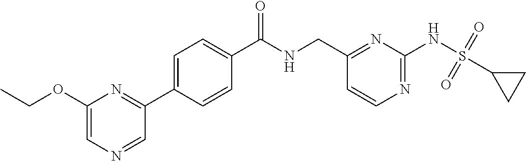 [SMILES: CCOc1cncc(-c2ccc(C(=O)NCc3ccnc(NS(=O)(=O)C4CC4)n3)cc2)n1] |
| P122 | 2-(2-(cyclopropanesulfonamido)pyrimidin- |
| | 4-yl)-2-methyl-N-(5-(6-(prop-1-en-2- |
| | yl)pyrazin-2-yl)pyridin-2-yl)propanamide |
| | 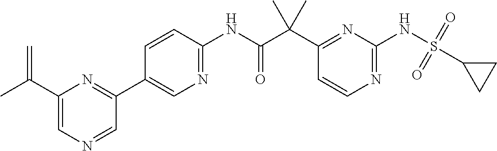 [SMILES: C=C(C)c1cncc(-c2ccc(NC(=O)C(C)(C)c3ccnc(NS(=O)(=O)C4CC4)n3)nc2)n1] |
| P123 | 2-(2-(cyclopropanesulfonamido)-6- |
| | methylpyrimidin-4-yl)-N-(5-(6- |
| | ethoxypyrazin-2-yl)pyridin-2-yl)-2- |
| | methylpropanamide |
| | 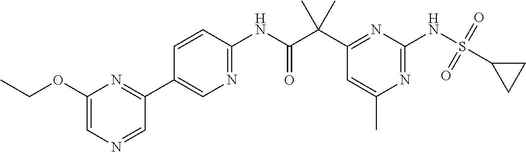 [SMILES: CCOc1cncc(-c2ccc(NC(=O)C(C)(C)c3cc(C)nc(NS(=O)(=O)C4CC4)n3)nc2)n1] |
| P124 | 2-(2-(cyclopropanesulfonamido)-6- |
| | (trifluoromethyl)pyrimidin-4-yl)-N-(5-(6- |
| | ethoxypyrazin-2-yl)pyridin-2-yl)-2- |
| | methylpropanamide |
| | 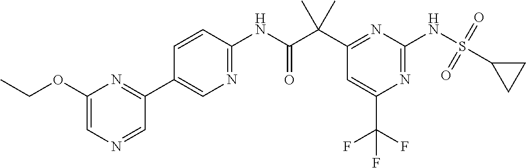 [SMILES: CCOc1cncc(-c2ccc(NC(=O)C(C)(C)c3cc(C(F)(F)F)nc(NS(=O)(=O)C4CC4)n3)nc2)n1] |
| P125 | 2-(2-(cyclopropanesulfonamido)pyrimidin- |
| | 4-yl)-N-(5-(6-cyclopropylpyrazin-2- |
| | yl)pyridin-2-yl)-2-methylpropanamide |
| | 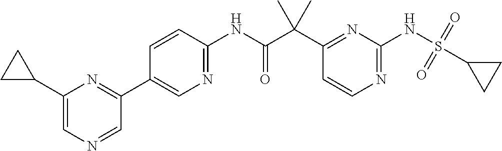 [SMILES: CC(C)(C(=O)Nc1ccc(-c2cncc(C3CC3)n2)cn1)c1ccnc(NS(=O)(=O)C2CC2)n1] |
| P126 | 2-(2-(cyclopropanesulfonamido)pyrimidin- |
| | 4-yl)-N-(6-(6-ethoxypyrazin-2-yl)pyridin-3- |
| | yl)-2-methylpropanamide |
| | 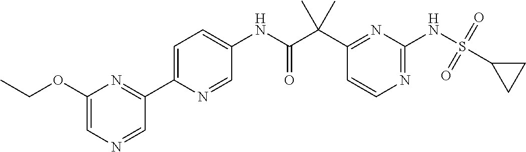 [SMILES: CCOc1cncc(-c2ccc(NC(=O)C(C)(C)c3ccnc(NS(=O)(=O)C4CC4)n3)cn2)n1] |
| P128 | 2-(2-(cyclopropanesulfonamido)pyrimidin- |
| | 4-yl)-N-(4-(9-cyclopropylpyrazin-2-yl)-2- |
| | fluorophenyl)-2-methylpropanamide |
| | 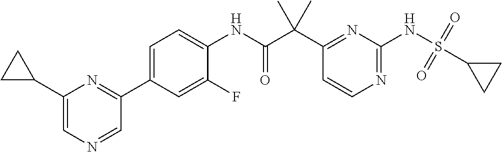 [SMILES: CC(C)(C(=O)Nc1ccc(-c2cncc(C3CC3)n2)cc1F)c1ccnc(NS(=O)(=O)C2CC2)n1] |
| P129 | 2-(2-(cyclopropanesulfonamido)-6- |
| | methylpyrimidin-4-yl)-N-(4-(6- |
| | ethoxypyrazin-2-yl)-2-fluorophenyl)-2- |
| | methylpropanamide |
| | 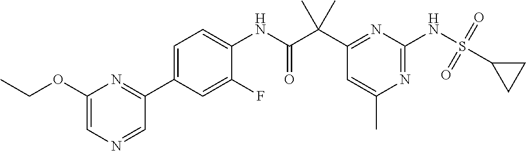 [SMILES: CCOc1cncc(-c2ccc(NC(=O)C(C)(C)c3cc(C)nc(NS(=O)(=O)C4CC4)n3)c(F)c2)n1] |
| P130 | 2-(2-(cyclopropanesulfonamido)-6- |
| | (trifluoromethyl)pyrimidin-4-yl)-N-(4-(6- |
| | ethoxypyrazin-2-yl)-2-fluorophenyl)-2- |
| | methylpropanamide |
| | 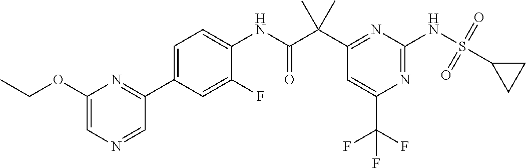 [SMILES: CCOc1cncc(-c2ccc(NC(=O)C(C)(C)c3cc(C(F)(F)F)nc(NS(=O)(=O)C4CC4)n3)c(F)c2)n1] |
| P131 | 2-(2-(cyclopropanesulfonamido)pyrimidin- |
| | 4-yl)-2-methyl-N-(4-(6-(prop-1-en-1- |
| | yl)pyrazin-2-yl)phenyl)propanamide |
| | 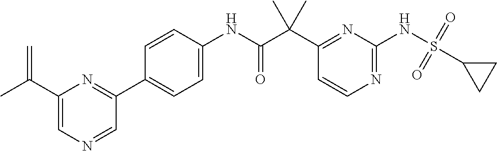 [SMILES: C=C(C)c1cncc(-c2ccc(NC(=O)C(C)(C)c3ccnc(NS(=O)(=O)C4CC4)n3)cc2)n1] |
| P132 | 2-(2-(cyclopropanesulfonamido)pyrimidin- |
| | 4-yl)-N-(4-(6-isopropylpyrazin-2- |
| | yl)phenyl)-2-methylpropanamide |
| | 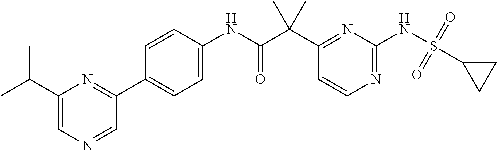 [SMILES: CC(C)c1cncc(-c2ccc(NC(=O)C(C)(C)c3ccnc(NS(=O)(=O)C4CC4)n3)cc2)n1] |
| P133 | 2-(2-(cyclopropanesulfonamido)pyrimidin- |
| | 4-yl)-N-(4-(6-(dimethylamino)pyrazin-2- |
| | yl)phenyl)-2-methylpropanamide |
| | 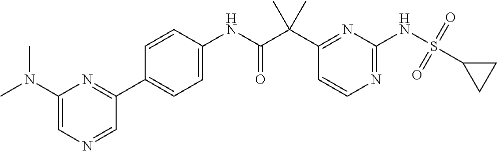 [SMILES: CN(C)c1cncc(-c2ccc(NC(=O)C(C)(C)c3ccnc(NS(=O)(=O)C4CC4)n3)cc2)n1] |
| P134 | 2-(2-(cyclopropanesulfonamido)-6- |
| | methylpyrimidin-4-yl)-N-(4-(6- |
| | ethoxypyrazin-2-yl)phenyl)-2- |
| | methylpropanamide |
| | 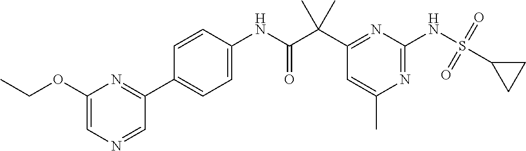 [SMILES: CCOc1cncc(-c2ccc(NC(=O)C(C)(C)c3cc(C)nc(NS(=O)(=O)C4CC4)n3)cc2)n1] |
| P135 | 2-(2-(cyclopropanesulfonamido)-6- |
| | (trifluoromethyl)pyrimidin-4-yl)-N-(4-(6- |
| | ethoxypyrazin-2-yl)phenyl)-2- |
| | methylpropanamide |
| | 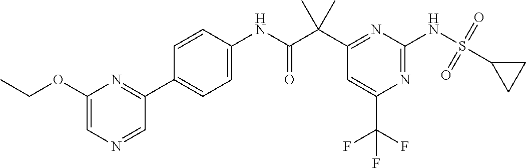 [SMILES: CCOc1cncc(-c2ccc(NC(=O)C(C)(C)c3cc(C(F)(F)F)nc(NS(=O)(=O)C4CC4)n3)cc2)n1] |
| P127 | 2-(2-(Cyclopropanesulfonamido)-6- |
| | methoxypyrimidin-4-yl)-2-methyl-N-(4- |
| | (pyridin-3-yl)phenyl)propanamide |
| | 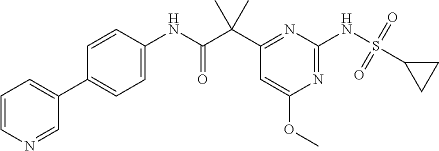 [SMILES: COc1cc(C(C)(C)C(=O)Nc2ccc(-c3cccnc3)cc2)nc(NS(=O)(=O)C2CC2)n1] |
| P136 | 1-(2-(cyclopropanesulfonamido)pyrimidin- |
| | 4-yl)-N-(5-(6-ethoxypyrazin-2-yl)pyridin-2- |
| | yl)cyclopentane-1-carboxamide |
| | 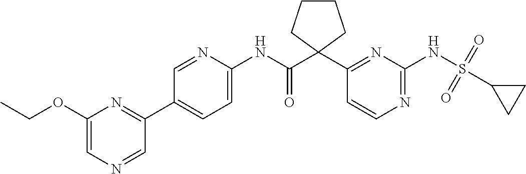 [SMILES: CCOc1cncc(-c2ccc(NC(=O)C3(c4ccnc(NS(=O)(=O)C5CC5)n4)CCCC3)nc2)n1] |
| P137 | 4-(2-(cyclopropanesulfonamido)pyrimidin- |
| | 4-yl)-N-(4-(6-ethoxypyrazin-2- |
| | yl)phenyl)tetrahydro-2H-pyran-4- |
| | carboxamide |
| | 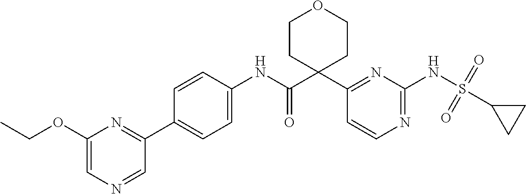 [SMILES: CCOc1cncc(-c2ccc(NC(=O)C3(c4ccnc(NS(=O)(=O)C5CC5)n4)CCOCC3)cc2)n1] |
| P138 | N-(5-(6-ethoxypyrazin-2-yl)pyridin-2-yl)-4- |
| | (2-(methylsulfonamido)pyrimidin-4- |
| | yl)piperidine-4-carboxamide |
| | 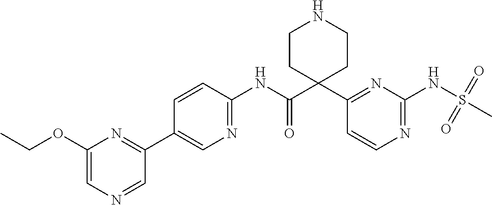 [SMILES: CCOc1cncc(-c2ccc(NC(=O)C3(c4ccnc(NS(C)(=O)=O)n4)CCNCC3)nc2)n1] |
| P139 | tert-butyl 4-(2- |
| | (cyclopropanesulfonamido)pyrimidin-4-yl)- |
| | 4-((5-(6-ethoxypyrazin-2-yl)pyridin-2- |
| | yl)carbamoyl)piperidine-1-carboxylate |
| | 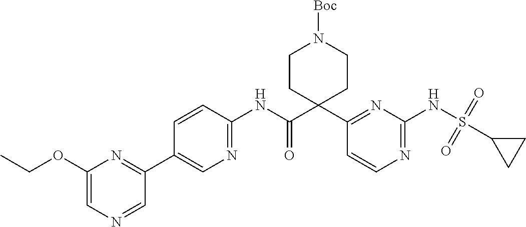 [SMILES: CCOc1cncc(-c2ccc(NC(=O)C3(c4ccnc(NS(=O)(=O)C5CC5)n4)CCN(C(=O)OC(C)(C)C)CC3)nc2)n1] |
| P140 | 4-(2-(cyclopropanesulfonamido)pyrimidin- |
| | 4-yl)-N-(5-(6-ethoxypyrazin-2-yl)pyridin-2- |
| | yl)piperidine-4-carboxamide |
| | 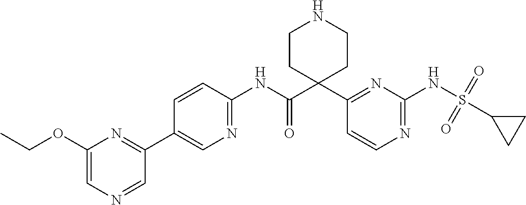 [SMILES: CCOc1cncc(-c2ccc(NC(=O)C3(c4ccnc(NS(=O)(=O)C5CC5)n4)CCNCC3)nc2)n1] |
| P141 | tert-butyl 3-(2- |
| | (cyclopropanesulfonamido)pyrimidin-4-yl)- |
| | 3-((5-(6-ethoxypyrazin-2-yl)pyridin-2- |
| | yl)carbamoyl)azetidine-1-carboxylate |
| | 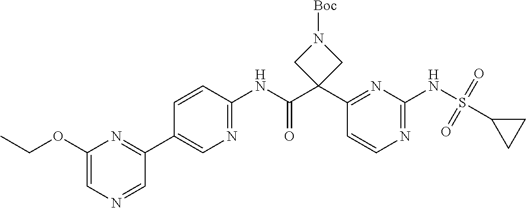 [SMILES: CCOc1cncc(-c2ccc(NC(=O)C3(c4ccnc(NS(=O)(=O)C5CC5)n4)CN(C(=O)OC(C)(C)C)C3)nc2)n1] |
| P142 | tert-butyl 4-((5-(6-ethoxypyrazin-2- |
| | yl)pyridin-2-yl)carbamoyl)-4-(2- |
| | (methylsulfonamido)pyrimidin-4- |
| | yl)piperidine-1-carboxylate |
| | 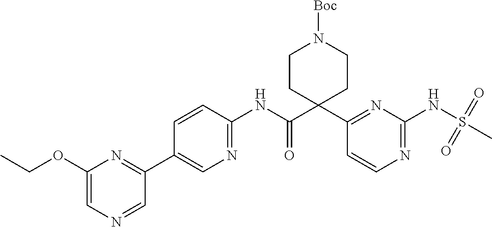 [SMILES: CCOc1cncc(-c2ccc(NC(=O)C3(c4ccnc(NS(C)(=O)=O)n4)CCN(C(=O)OC(C)(C)C)CC3)nc2)n1] |
| P143 | 4-(2-(cyclopropanesulfonamido)pyrimidin- |
| | 4-yl)-N-(4-(6-ethoxypyrazin-2-yl)-2- |
| | fluorophenyl)tetrahydro-2H-pyran-4- |
| | carboxamide |
| | 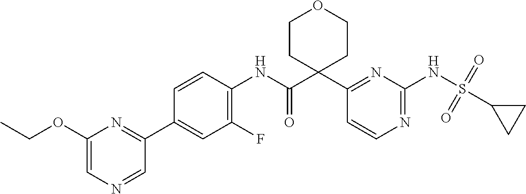 [SMILES: CCOc1cncc(-c2ccc(NC(=O)C3(c4ccnc(NS(=O)(=O)C5CC5)n4)CCOCC3)c(F)c2)n1] |
| P144 | 2-(2-(cyclopropanesulfonamido)pyrimidin- |
| | 4-yl)-N-(5-(6-ethoxypyrazin-2-yl)-3- |
| | fluoropyridin-2-yl)-4-methoxybutanamide |
| | 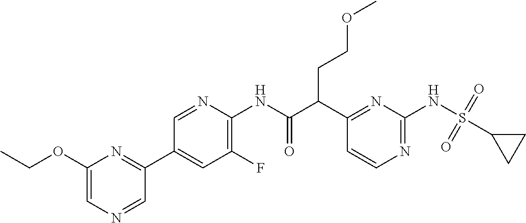 [SMILES: CCOc1cncc(-c2cnc(NC(=O)C(CCOC)c3ccnc(NS(=O)(=O)C4CC4)n3)c(F)c2)n1] |
| P145 | 2-(2-(cyclopropanesulfonamido)pyrimidin- |
| | 4-yl)-N-(5-(6-ethoxypyrazin-2-yl)pyridin-2- |
| | yl)-4-methoxybutanamide |
| | 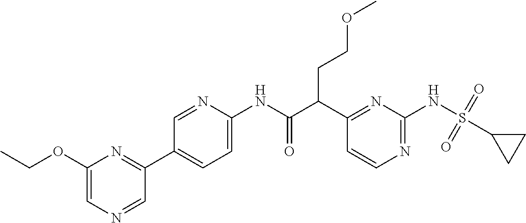 [SMILES: CCOc1cncc(-c2ccc(NC(=O)C(CCOC)c3ccnc(NS(=O)(=O)C4CC4)n3)nc2)n1] |
| P146 | 2-(2-(cyclopropanesulfonamido)pyrimidin- |
| | 4-yl)-N-(4-(6-ethoxypyrazin-2-yl)-2- |
| | fluorophenyl)-4-methoxybutanamide |
| | 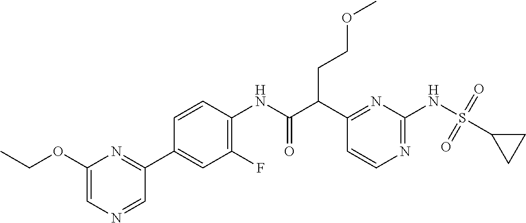 [SMILES: CCOc1cncc(-c2ccc(NC(=O)C(CCOC)c3ccnc(NS(=O)(=O)C4CC4)n3)c(F)c2)n1] |
| P147 | N-(5-(6-ethoxypyrazin-2-yl)pyridin-2-yl)-4- |
| | methoxy-2-methyl-2-(2- |
| | (methylsulfonamido)pyrimidin-4- |
| | yl)butanamide |
| | 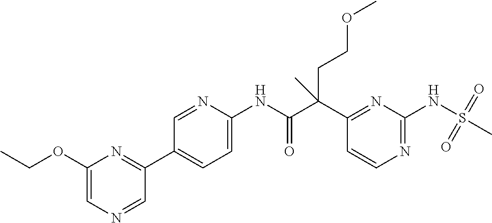 [SMILES: CCOc1cncc(-c2ccc(NC(=O)C(C)(CCOC)c3ccnc(NS(C)(=O)=O)n3)nc2)n1] |
| P148 | N-(5′-chloro-[3,3′-bipyridin]-6-yl)-2-(2- |
| | (cyclopropanesulfonamido)pyrimidin-4- |
| | yl)butanamide |
| | 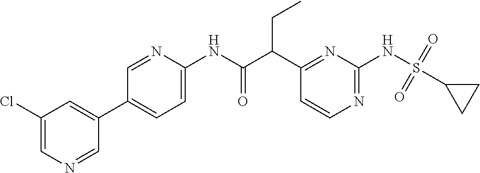 [SMILES: CCC(C(=O)Nc1ccc(-c2cncc(Cl)c2)cn1)c1ccnc(NS(=O)(=O)C2CC2)n1] |
| P149 | N-(5′-chloro-[3,3′-bipyridin]-6-yl)-2-(2- |
| | (cyclopropanesulfonamido)pyrimidin-4-yl)- |
| | 2-fluorobutanamide |
| | 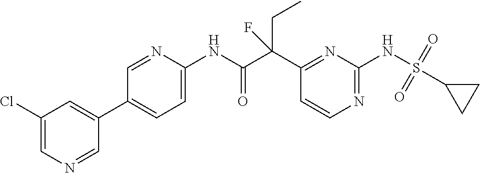 [SMILES: CCC(F)(C(=O)Nc1ccc(-c2cncc(Cl)c2)cn1)c1ccnc(NS(=O)(=O)C2CC2)n1] |
| P150 | 2-(2-(cyclopropanesulfonamido)pyrimidin- |
| | 4-yl)-N-(5-(6-cyclopropylpyrazin-2- |
| | yl)pyridin-2-yl)-2-fluorobutanamide |
| | 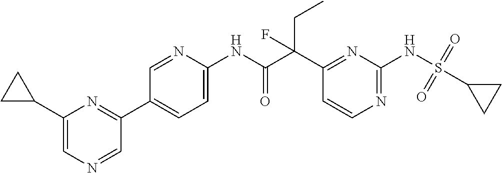 [SMILES: CCC(F)(C(=O)Nc1ccc(-c2cncc(C3CC3)n2)cn1)c1ccnc(NS(=O)(=O)C2CC2)n1] |
| P151 | N-(5-(6-ethoxypyrazin-2-yl)pyridin-2-yl)-2- |
| | fluoro-2-(2-(methylsulfonamido)pyrimidin- |
| | 4-yl)butanamide |
| | 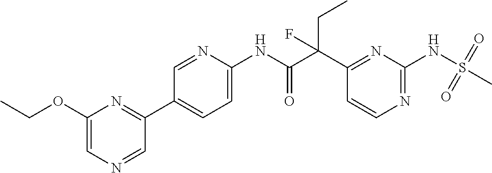 [SMILES: CCOc1cncc(-c2ccc(NC(=O)C(F)(CC)c3ccnc(NS(C)(=O)=O)n3)nc2)n1] |
| P155 | 2-(2-(cyclopropanesulfonamido)pyrimidin- |
| | 4-yl)-N-(5-(6-ethoxypyrazin-2-yl)-3- |
| | methylpyridin-2-yl)butanamide |
| | 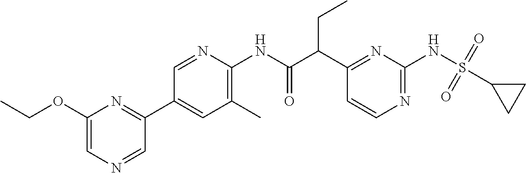 [SMILES: CCOc1cncc(-c2cnc(NC(=O)C(CC)c3ccnc(NS(=O)(=O)C4CC4)n3)c(C)c2)n1] |
| P156 | 2-(2-(cyclopropanesulfonamido)pyrimidin- |
| | 4-yl)-N-(5-(6-cyclopropylpyrazin-2- |
| | yl)pyridin-2-yl)butanamide |
| | 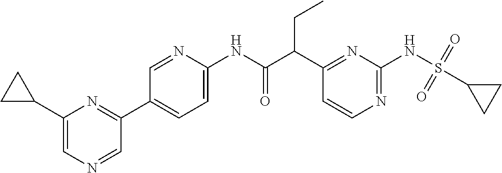 [SMILES: CCC(C(=O)Nc1ccc(-c2cncc(C3CC3)n2)cn1)c1ccnc(NS(=O)(=O)C2CC2)n1] |
| P157 | 2-(2-(cyclopropanesulfonamido)pyrimidin- |
| | 4-yl)-N-(5-(6-(2,2,2- |
| | trifluoroethoxy)pyrazin-2-yl)pyridin-2- |
| | yl)butanamide |
| | 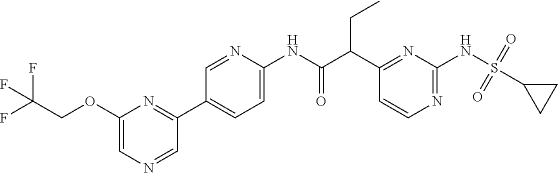 [SMILES: CCC(C(=O)Nc1ccc(-c2cncc(OCC(F)(F)F)n2)cn1)c1ccnc(NS(=O)(=O)C2CC2)n1] |
| P158 | 2-(2-(cyclopropanesulfonamido)pyrimidin- |
| | 4-yl)-N-(3-fluoro-5-(6-methoxypyrazin-2- |
| | yl)pyridin-2-yl)butanamide |
| | 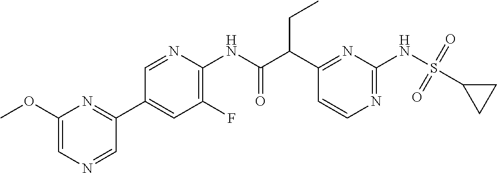 [SMILES: CCC(C(=O)Nc1ncc(-c2cncc(OC)n2)cc1F)c1ccnc(NS(=O)(=O)C2CC2)n1] |
| P159 | 2-(2-(cyclopropanesulfonamido)pyrimidin- |
| | 4-yl)-N-(5-(6-methoxypyrazin-2-yl)pyridin- |
| | 2-yl)butanamide |
| | 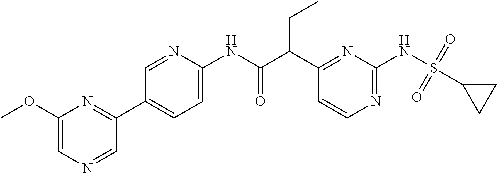 [SMILES: CCC(C(=O)Nc1ccc(-c2cncc(OC)n2)cn1)c1ccnc(NS(=O)(=O)C2CC2)n1] |
| P160 | 2-(2-(cyclopropanesulfonamido)pyrimidin- |
| | 4-yl)-N-(4-(6-cyclopropylpyrazin-2-yl)-2- |
| | fluorophenyl)butanamide |
| | 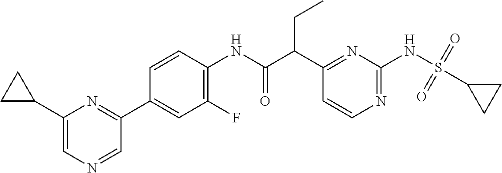 [SMILES: CCC(C(=O)Nc1ccc(-c2cncc(C3CC3)n2)cc1F)c1ccnc(NS(=O)(=O)C2CC2)n1] |
| P161 | 2-(2-(cyclopropanesulfonamido)pyrimidin- |
| | 4-yl)-N-(4-(9-ethoxypyrazin-2-yl)-2- |
| | methylphenyl)butanamide |
| | 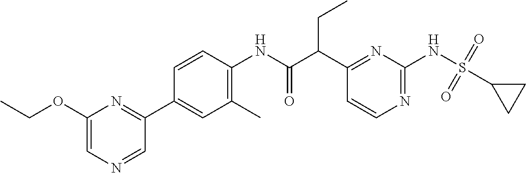 [SMILES: CCOc1cncc(-c2ccc(NC(=O)C(CC)c3ccnc(NS(=O)(=O)C4CC4)n3)c(C)c2)n1] |
| P162 | 2-(2-(cyclopropanesulfonamido)pyrimidin- |
| | 4-yl)-N-(5-(6-ethoxypyrazin-2-yl)-3- |
| | fluoropyridin-2-yl)butanamide |
| | 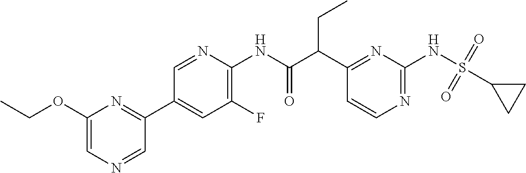 [SMILES: CCOc1cncc(-c2cnc(NC(=O)C(CC)c3ccnc(NS(=O)(=O)C4CC4)n3)c(F)c2)n1] |
| P163 | 2-(2-(cyclopropanesulfonamido)pyrimidin- |
| | 4-yl)-N-(5-(6-ethoxypyrazin-2-yl)pyridin-2- |
| | yl)-2-methylbutanamide |
| | 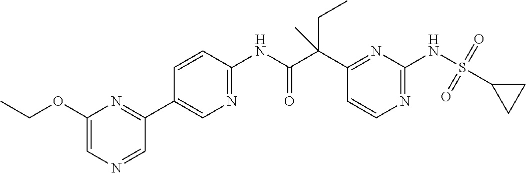 [SMILES: CCOc1cncc(-c2ccc(NC(=O)C(C)(CC)c3ccnc(NS(=O)(=O)C4CC4)n3)nc2)n1] |
| P152 | 2-(2-(cyclopropanesulfonamido)pyrimidin- |
| | 4-yl)-N-(5-(6-ethoxypyrazin-2-yl)pyridin-2- |
| | yl)-2-fluoro-3-methylbutanamide |
| | 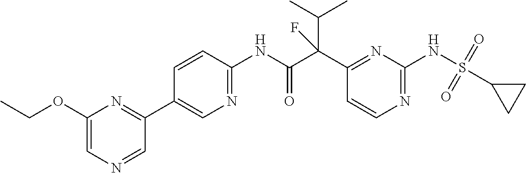 [SMILES: CCOc1cncc(-c2ccc(NC(=O)C(F)(c3ccnc(NS(=O)(=O)C4CC4)n3)C(C)C)nc2)n1] |
| P153 | 2-(2-(cyclopropanesulfonamido)pyrimidin- |
| | 4-yl)-N-(4-(6-ethoxypyrazin-2-yl)-2- |
| | fluorophenyl)-3-methylbutanamide |
| | 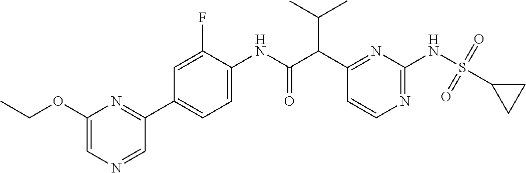 [SMILES: CCOc1cncc(-c2ccc(NC(=O)C(c3ccnc(NS(=O)(=O)C4CC4)n3)C(C)C)c(F)c2)n1] |
| P154 | 2-(2-(cyclopropanesulfonamido)pyrimidin- |
| | 4-yl)-N-(5-(6-ethoxypyrazin-2-yl)pyridin-2- |
| | yl)-3-methylbutanamide |
| | 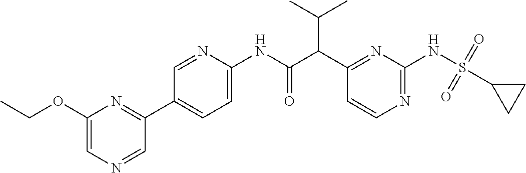 [SMILES: CCOc1cncc(-c2ccc(NC(=O)C(c3ccnc(NS(=O)(=O)C4CC4)n3)C(C)C)nc2)n1] |
| P164 | 2-(2-(cyclopropanesulfonamido)pyrimidin- |
| | 4-yl)-N-(4-(6-ethoxypyrazin-2-yl)phenyl)- |
| | 2-methoxyacetamide |
| | 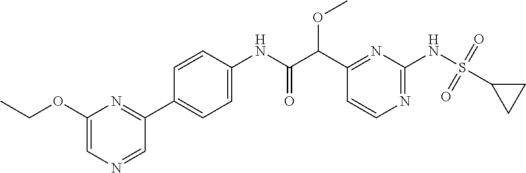 [SMILES: CCOc1cncc(-c2ccc(NC(=O)C(OC)c3ccnc(NS(=O)(=O)C4CC4)n3)cc2)n1] |
| P165 | Single enantiomer - stereochemistry |
| | unassigned |
| | N-(5-(6-ethoxypyrazin-2-yl)pyridin-2-yl)-2- |
| | fluoro-2-(2-(methylsulfonamido)pyrimidin- |
| | 4-yl)butanamide |
| | 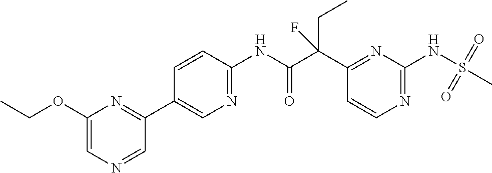 [SMILES: CCOc1cncc(-c2ccc(NC(=O)C(F)(CC)c3ccnc(NS(C)(=O)=O)n3)nc2)n1] |
| P166 | Single enantiomer - stereochemistry |
| | unassigned |
| | N-(5-(6-ethoxypyrazin-2-yl)pyridin-2-yl)-2- |
| | fluoro-2-(2-(methylsulfonamido)pyrimidin- |
| | 4-yl)butanamide |
| | 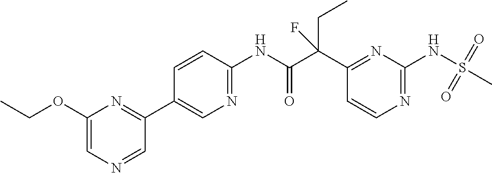 [SMILES: CCOc1cncc(-c2ccc(NC(=O)C(F)(CC)c3ccnc(NS(C)(=O)=O)n3)nc2)n1] |
| P167 | N-(4-(5-chloropyridin-3-yl)phenyl)-2-(6- |
| | (cyclopropanesulfonamido)pyridin-2- |
| | yl)acetamide |
| | 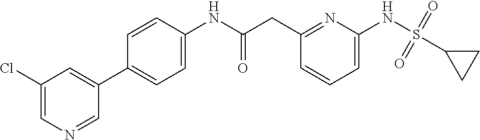 [SMILES: O=C(Cc1cccc(NS(=O)(=O)C2CC2)n1)Nc1ccc(-c2cncc(Cl)c2)cc1] |
| P168 | N-(4-(5-cyanopyridin-3-yl)phenyl)-2-(6- |
| | (cyclopropanesulfonamido)pyridin-2- |
| | yl)acetamide |
| | 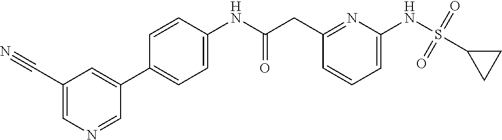 [SMILES: N#Cc1cncc(-c2ccc(NC(=O)Cc3cccc(NS(=O)(=O)C4CC4)n3)cc2)c1] |
| P169 | 2-(6-(cyclopropanesulfonamido)pyridin-2- |
| | yl)-N-(4-(5-fluoropyridin-3- |
| | yl)phenyl)acetamide |
| | 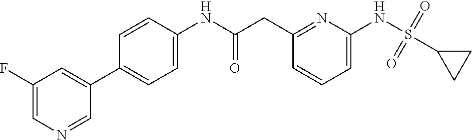 [SMILES: O=C(Cc1cccc(NS(=O)(=O)C2CC2)n1)Nc1ccc(-c2cncc(F)c2)cc1] |
| P170 | 2-(6-(cyclopropanesulfonamido)pyridin-2- |
| | yl)-N-(4-(6-methoxypyridin-3- |
| | yl)phenyl)acetamide |
| | 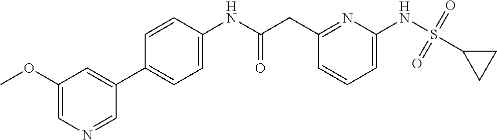 [SMILES: COc1cncc(-c2ccc(NC(=O)Cc3cccc(NS(=O)(=O)C4CC4)n3)cc2)c1] |
| P171 | 2-(6-(cyclopropanesulfonamido)pyridin-2- |
| | yl)-N-(4-(pyridin-3-yl)phenyl)acetamide |
| | 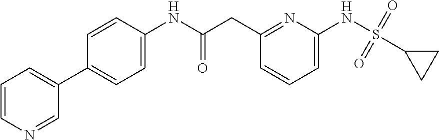 [SMILES: O=C(Cc1cccc(NS(=O)(=O)C2CC2)n1)Nc1ccc(-c2cccnc2)cc1] |
| P172 | 2-(6-(cyclopropanesulfonamido)pyridin-2- |
| | yl)-N-(4-(6-(trifluoromethyl)pyrazin-2- |
| | yl)phenyl)acetamide |
| | 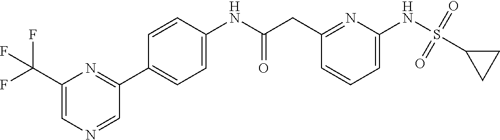 [SMILES: O=C(Cc1cccc(NS(=O)(=O)C2CC2)n1)Nc1ccc(-c2cncc(C(F)(F)F)n2)cc1] |
| P173 | 2-(6-(cyclopropanesulfonamido)pyridin-2- |
| | yl)-N-(4-(6-methoxypyrazin-2- |
| | yl)phenyl)acetamide |
| | 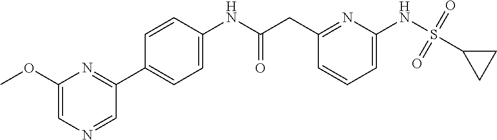 [SMILES: COc1cncc(-c2ccc(NC(=O)Cc3cccc(NS(=O)(=O)C4CC4)n3)cc2)n1] |
| P174 | 2-(6-(cyclopropanesulfonamido)pyridin-2- |
| | yl)-N-(4-(pyrazin-2-yl)phenyl)acetamide |
| | 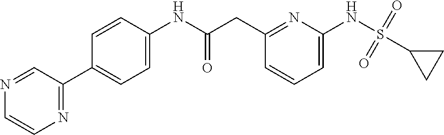 [SMILES: O=C(Cc1cccc(NS(=O)(=O)C2CC2)n1)Nc1ccc(-c2cnccn2)cc1] |
| P175 | N-([3,3′-bipyridin]-6-yl)-2-(6- |
| | (cyclopropanesulfonamido)pyridin-2-yl)-2- |
| | methylpropanamide |
| | 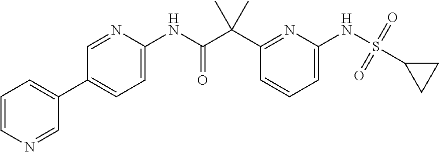 [SMILES: CC(C)(C(=O)Nc1ccc(-c2cccnc2)cn1)c1cccc(NS(=O)(=O)C2CC2)n1] |
| P176 | N-(4-(5-chloropyridin-3-yl)phenyl)-2-(6- |
| | (cyclopropanesulfonamido)pyridin-2-yl)-2- |
| | methylpropanamide |
| | 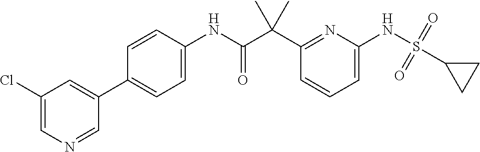 [SMILES: CC(C)(C(=O)Nc1ccc(-c2cncc(Cl)c2)cc1)c1cccc(NS(=O)(=O)C2CC2)n1] |
| P177 | 2-(6-(cyclopropanesulfonamido)pyridin-2- |
| | yl)-N-(4-(5-fluoropyridin-3-yl)phenyl)-2- |
| | methylpropanamide |
| | 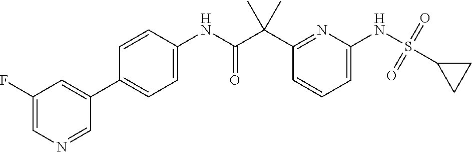 [SMILES: CC(C)(C(=O)Nc1ccc(-c2cncc(F)c2)cc1)c1cccc(NS(=O)(=O)C2CC2)n1] |
| P178 | 2-(6-(cyclopropanesulfonamido)pyridin-2- |
| | yl)-N-(4-(5-ethoxypyridin-3-yl)phenyl)-2- |
| | methylpropanamide |
| | 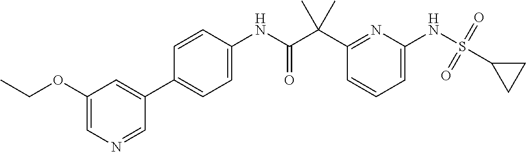 [SMILES: CCOc1cncc(-c2ccc(NC(=O)C(C)(C)c3cccc(NS(=O)(=O)C4CC4)n3)cc2)c1] |
| P179 | 2-(6-(cyclopropanesulfonamido)pyridin-2- |
| | yl)-2-methyl-N-(4-(pyridin-3- |
| | yl)phenyl)propanamide |
| | 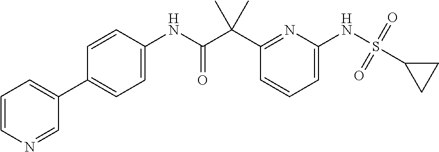 [SMILES: CC(C)(C(=O)Nc1ccc(-c2cccnc2)cc1)c1cccc(NS(=O)(=O)C2CC2)n1] |
| P180 | 2-(6-(cyclopropanesulfonamido)pyridin-2- |
| | yl)-N-(2-fluoro-4-(pyrazin-2-yl)phenyl)-2- |
| | methylpropanamide |
| | 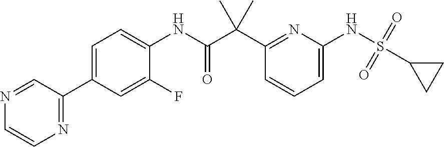 [SMILES: CC(C)(C(=O)Nc1ccc(-c2cnccn2)cc1F)c1cccc(NS(=O)(=O)C2CC2)n1] |
| P181 | 2-(6-(cyclopropanesulfonamido)pyridin-2- |
| | yl)-2-methyl-N-(4-(6- |
| | (trifluoromethyl)pyrazin-2- |
| | yl)phenyl)propanamide |
| | 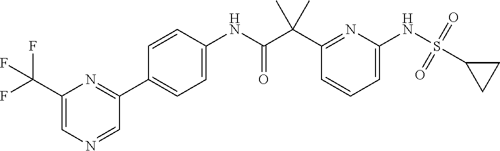 [SMILES: CC(C)(C(=O)Nc1ccc(-c2cncc(C(F)(F)F)n2)cc1)c1cccc(NS(=O)(=O)C2CC2)n1] |
| P182 | N-(4-(6-chloropyrazin-2-yl)phenyl)-2-(6- |
| | (cyclopropanesulfonamido)pyridin-2-yl)-2- |
| | methylpropanamide |
| | 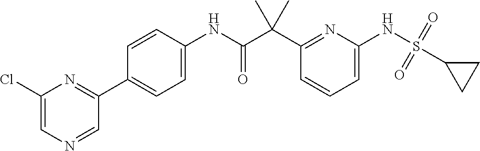 [SMILES: CC(C)(C(=O)Nc1ccc(-c2cncc(Cl)n2)cc1)c1cccc(NS(=O)(=O)C2CC2)n1] |
| P183 | 2-(6-(cyclopropanesulfonamido)pyridin-2- |
| | yl)-N-(4-(6-ethoxypyrazin-2-yl)phenyl)-2- |
| | methylpropanamide |
| | 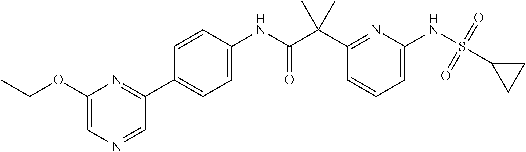 [SMILES: CCOc1cncc(-c2ccc(NC(=O)C(C)(C)c3cccc(NS(=O)(=O)C4CC4)n3)cc2)n1] |
| P184 | 2-(6-(cyclopropanesulfonamido)pyridin-2- |
| | yl)-N-(4-(6-methoxypyrazin-2-yl)phenyl)-2- |
| | methylpropanamide |
| | 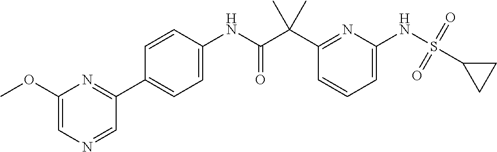 [SMILES: COc1cncc(-c2ccc(NC(=O)C(C)(C)c3cccc(NS(=O)(=O)C4CC4)n3)cc2)n1] |
| P185 | 2-(6-(cyclopropanesulfonamido)pyridin-2- |
| | yl)-2-methyl-N-(4-(pyrazin-2- |
| | yl)phenyl)propanamide |
| | 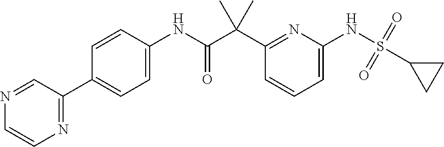 [SMILES: CC(C)(C(=O)Nc1ccc(-c2cnccn2)cc1)c1cccc(NS(=O)(=O)C2CC2)n1] |
| P186 | 4-(6-(cyclopropanesulfonamido)pyridin-2- |
| | yl)-N-(5-(6-ethoxypyrazin-2-yl)pyridin-2- |
| | yl)tetrahydro-2H-pyran-4-carboxamide |
| | 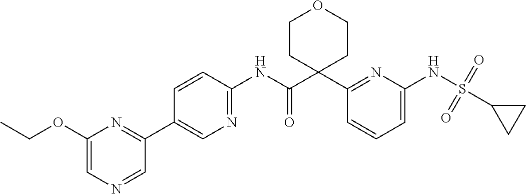 [SMILES: CCOc1cncc(-c2ccc(NC(=O)C3(c4cccc(NS(=O)(=O)C5CC5)n4)CCOCC3)nc2)n1] |
| P187 | 2-(6-(cyclopropanesulfonamido)pyridin-2- |
| | yl)-N-(5-(6-(trifluoromethyl)pyrazin-2- |
| | yl)pyridin-2-yl)butanamide |
| | 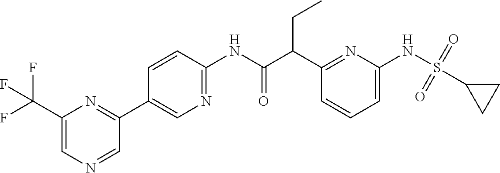 [SMILES: CCC(C(=O)Nc1ccc(-c2cncc(C(F)(F)F)n2)cn1)c1cccc(NS(=O)(=O)C2CC2)n1] |
| P188 | 2-(6-(cyclopropanesulfonamido)pyridin-2- |
| | yl)-N-(5-(6-ethoxypyrazin-2-yl)pyridin-2- |
| | yl)butanamide |
| | 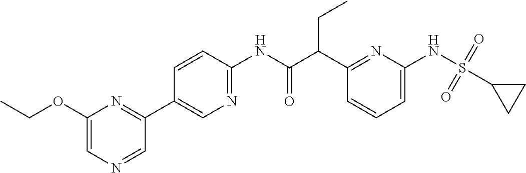 [SMILES: CCOc1cncc(-c2ccc(NC(=O)C(CC)c3cccc(NS(=O)(=O)C4CC4)n3)nc2)n1] |
| P189 | N-(4-(5-chloropyridin-3-yl)phenyl)-2-(6- |
| | (cyclopropanesulfonamido)pyridin-2- |
| | yl)butanamide |
| | 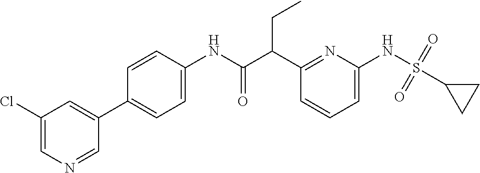 [SMILES: CCC(C(=O)Nc1ccc(-c2cncc(Cl)c2)cc1)c1cccc(NS(=O)(=O)C2CC2)n1] |
| P190 | 2-(6-(cyclopropanesulfonamido)pyridin-2- |
| | yl)-N-(4-(6-ethoxypyrazin-2-yl)-2- |
| | fluorophenyl)butanamide |
| | 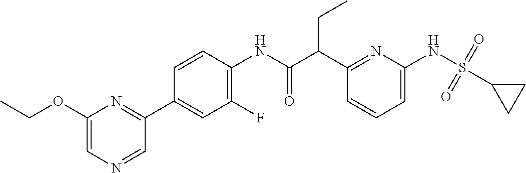 [SMILES: CCOc1cncc(-c2ccc(NC(=O)C(CC)c3cccc(NS(=O)(=O)C4CC4)n3)c(F)c2)n1] |
| P191 | 2-(6-(cyclopropanesulfonamido)pyridin-2- |
| | yl)-N-(4-(6-ethoxypyrazin-2- |
| | yl)phenyl)butanamide |
| | 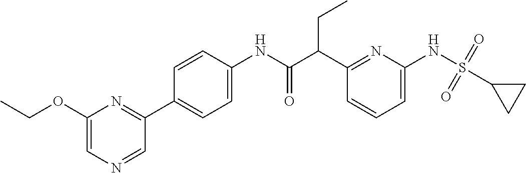 [SMILES: CCOc1cncc(-c2ccc(NC(=O)C(CC)c3cccc(NS(=O)(=O)C4CC4)n3)cc2)n1] |
| P192 | 2-(6-(cyclopropanesulfonamido)pyrazin-2- |
| | yl)-N-(4-(pyridin-3-yl)phenyl)acetamide |
| | 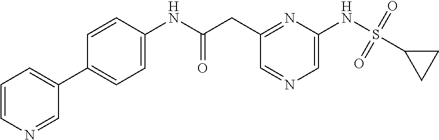 [SMILES: O=C(Cc1cncc(NS(=O)(=O)C2CC2)n1)Nc1ccc(-c2cccnc2)cc1] |
| P193 | 2-(6-(ethylsulfonamido)pyrazin-2-yl)-N-(4- |
| | (pyridin-3-yl)phenyl)acetamide |
| | 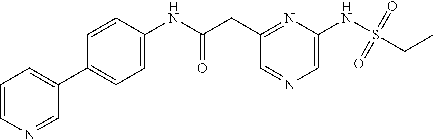 [SMILES: CCS(=O)(=O)Nc1cncc(CC(=O)Nc2ccc(-c3cccnc3)cc2)n1] |
| P194 | 2-(6-(methylsulfonamido)pyrazin-2-yl)-N- |
| | (4-(pyridin-3-yl)phenyl)acetamide |
| | 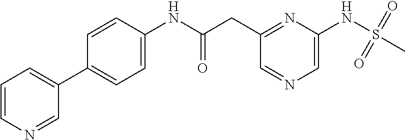 [SMILES: CS(=O)(=O)Nc1cncc(CC(=O)Nc2ccc(-c3cccnc3)cc2)n1] |
| P195 | 2-(6-(cyclopropanesulfonamido)pyrazin-2- |
| | yl)-N-(5-(6-ethoxypyrazin-2-yl)pyridin-2- |
| | yl)-2-methylpropanamide |
| | 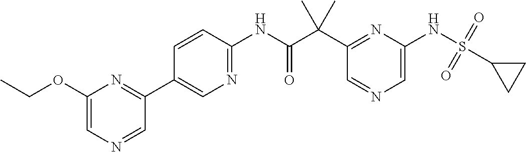 [SMILES: CCOc1cncc(-c2ccc(NC(=O)C(C)(C)c3cncc(NS(=O)(=O)C4CC4)n3)nc2)n1] |
| P196 | 2-(6-(cyclopropanesulfonamido)pyrazin-2- |
| | yl)-N-(4-(6-ethoxypyrazin-2-yl)phenyl)-2- |
| | methylpropanamide |
| | 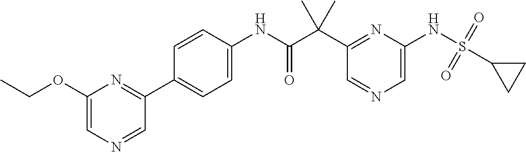 [SMILES: CCOc1cncc(-c2ccc(NC(=O)C(C)(C)c3cncc(NS(=O)(=O)C4CC4)n3)cc2)n1] |
| P197 | 4-(6-(cyclopropanesulfonamido)pyrazin-2- |
| | yl)-N-(5-(6-ethoxypyrazin-2-yl)pyridin-2- |
| | yl)tetrahydro-2H-pyran-4-carboxmaide |
| | 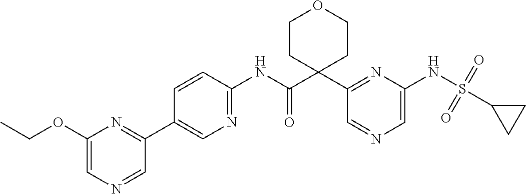 [SMILES: CCOc1cncc(-c2ccc(NC(=O)C3(c4cncc(NS(=O)(=O)C5CC5)n4)CCOCC3)nc2)n1] |
| P198 | 2-(6-(cyclopropanesulfonamido)pyrazin-2- |
| | yl)-N-(5-(6-ethoxypyrazin-2-yl)pyridin-2- |
| | yl)-4-methoxy-2-methylbutanamide |
| | 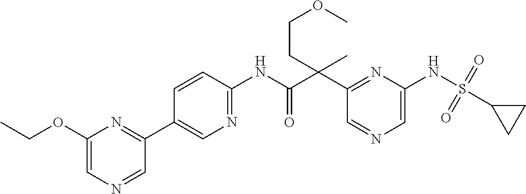 [SMILES: CCOc1cncc(-c2ccc(NC(=O)C(C)(CCOC)c3cncc(NS(=O)(=O)C4CC4)n3)nc2)n1] |
| P199 | N-(5-(6-ethoxypyrazin-2-yl)pyridin-2-yl)-4- |
| | methoxy-2-methyl-2-(6- |
| | (methylsulfonamido)pyrazin-2- |
| | yl)butanamide |
| | 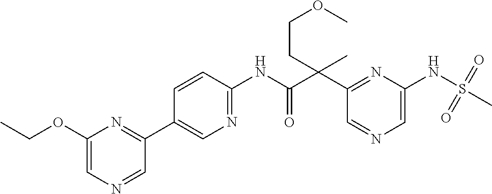 [SMILES: CCOc1cncc(-c2ccc(NC(=O)C(C)(CCOC)c3cncc(NS(C)(=O)=O)n3)nc2)n1] |
| P200 | 2-(6-(cyclopropanesulfonamido)pyrazin-2- |
| | yl)-N-(5-(6-ethoxypyrazin-2-yl)pyridin-2- |
| | yl)-2-fluorobutanamide |
| | 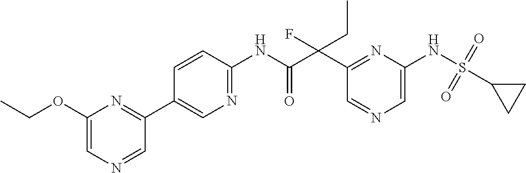 [SMILES: CCOc1cncc(-c2ccc(NC(=O)C(F)(CC)c3cncc(NS(=O)(=O)C4CC4)n3)nc2)n1] |
| P201 | 2-(6-(cyclopropanesulfonamido)pyrazin-2- |
| | yl)-N-(5-(6-ethoxypyrazin-2-yl)pyridin-2- |
| | yl)butanamide |
| | 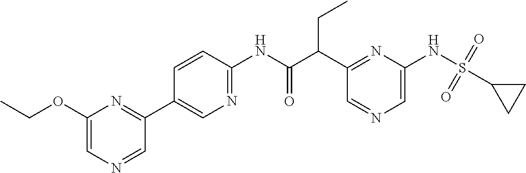 [SMILES: CCOc1cncc(-c2ccc(NC(=O)C(CC)c3cncc(NS(=O)(=O)C4CC4)n3)nc2)n1] |
| P202 | 2-(6-(cyclopropanesulfonamido)pyrazin-2- |
| | yl)-N-(4-(6-ethoxypyrazin-2- |
| | yl)phenyl)butanamide |
| | 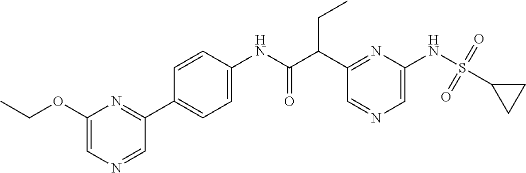 [SMILES: CCOc1cncc(-c2ccc(NC(=O)C(CC)c3cncc(NS(=O)(=O)C4CC4)n3)cc2)n1] |
| P203 | 2-(6-(cyclopropanesulfonamido)pyrazin-2- |
| | yl)-N-(5-(6-ethoxypyrazin-2-yl)pyridin-2- |
| | yl)-2-methoxyacetamide |
| | 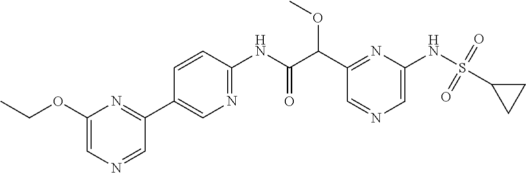 [SMILES: CCOc1cncc(-c2ccc(NC(=O)C(OC)c3cncc(NS(=O)(=O)C4CC4)n3)nc2)n1] |
| P204 | 2-(6-(cyclopropanesulfonamido)pyrazin-2- |
| | yl)-N-(4-(6-ethoxypyrazin-2-yl)phenyl)-2- |
| | methoxyacetamide |
| | 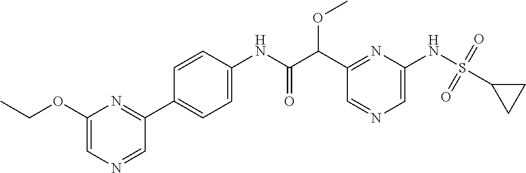 [SMILES: CCOc1cncc(-c2ccc(NC(=O)C(OC)c3cncc(NS(=O)(=O)C4CC4)n3)cc2)n1] |
| P205 | 2-(6-(cyclopropanesulfonamido)pyrazin-2- |
| | yl)-N-(5-(6-ethoxypyrazin-2-yl)pyridin-2- |
| | yl)-2-methoxypropanamide |
| | 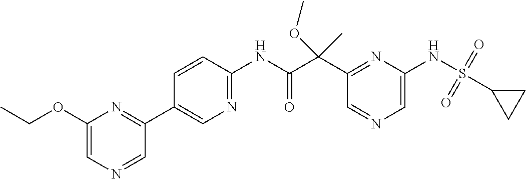 [SMILES: CCOc1cncc(-c2ccc(NC(=O)C(C)(OC)c3cncc(NS(=O)(=O)C4CC4)n3)nc2)n1] |
| P205a | Single enantiomer - stereochemistry |
| | unassigned |
| | 2-(6-(cyclopropanesulfonamido)pyrazin-2- |
| | yl)-N-(5-(6-ethoxypyrazin-2-yl)pyridin-2- |
| | yl)-2-methoxypropanamide |
| | 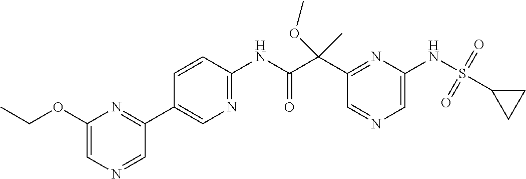 [SMILES: CCOc1cncc(-c2ccc(NC(=O)C(C)(OC)c3cncc(NS(=O)(=O)C4CC4)n3)nc2)n1] |
| P205b | Single enantiomer - stereochemistry |
| | unassigned |
| | 2-(6-(cyclopropanesulfonamido)pyrazin-2- |
| | yl)-N-(5-(6-ethoxypyrazin-2-yl)pyridin-2- |
| | yl)-2-methoxypropanamide |
| | 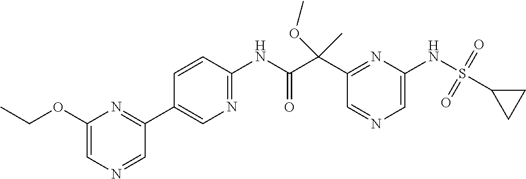 [SMILES: CCOc1cncc(-c2ccc(NC(=O)C(C)(OC)c3cncc(NS(=O)(=O)C4CC4)n3)nc2)n1] |
| P206 | Single enantiomer - stereochemistry |
| | unassigned |
| | 2-(6-(2-cyclopropanesulfonamido)pyrazin- |
| | 2-yl)-N-(5-(6-ethoxypyrazin-2-yl)pyridin-2- |
| | yl)-2-fluorobutanamide |
| | 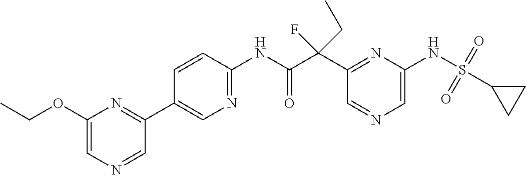 [SMILES: CCOc1cncc(-c2ccc(NC(=O)C(F)(CC)c3cncc(NS(=O)(=O)C4CC4)n3)nc2)n1] |
| P207 | Single enantiomer - stereochemistry |
| | unassigned |
| | 2-(6-(cyclopropanesulfonamido)pyrazin- |
| | 2-yl)-N-(5-(6-ethoxypyrazin-2-yl)pyridin-2- |
| | yl)-2-fluorobutanamide |
| | 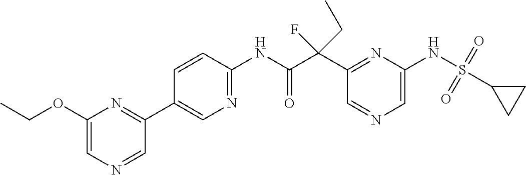 [SMILES: CCOc1cncc(-c2ccc(NC(=O)C(F)(CC)c3cncc(NS(=O)(=O)C4CC4)n3)nc2)n1] |
| P208 | 2-(4-(cyclopropanesulfonamido)pyrimidin- |
| | 2-yl)-N-(5-(6-ethoxypyrazin-2-yl)pyridin-2- |
| | yl)butanamide |
| | 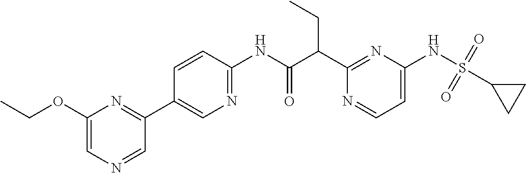 [SMILES: CCOc1cncc(-c2ccc(NC(=O)C(CC)c3nccc(NS(=O)(=O)C4CC4)n3)nc2)n1] |
| P209 | N-(1-(2- |
| | (cyclopropanesulfonamido)pyrimidin-4- |
| | yl)cyclopropyl)-4-(6-ethoxypyrazin-2-yl)-2- |
| | fluorobenzamide |
| | 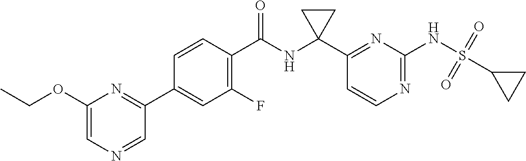 [SMILES: CCOc1cncc(-c2ccc(C(=O)NC3(c4ccnc(NS(=O)(=O)C5CC5)n4)CC3)c(F)c2)n1] |
| P210 | N-(1-(2- |
| | (cyclopropanesulfonamido)pyrimidin-4- |
| | yl)propyl)-5-(6-ethoxypyrazin-2- |
| | yl)picolinamide |
| | 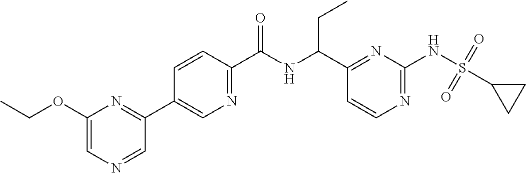 [SMILES: CCOc1cncc(-c2ccc(C(=O)NC(CC)c3ccnc(NS(=O)(=O)C4CC4)n3)nc2)n1] |
| P211 | N-(1-(2- |
| | (cyclopropanesulfonamido)pyrimidin-4- |
| | yl)propyl)-2-fluoro-4-(5- |
| | (trifluoromethyl)pyridin-3-yl)benzamide |
| | 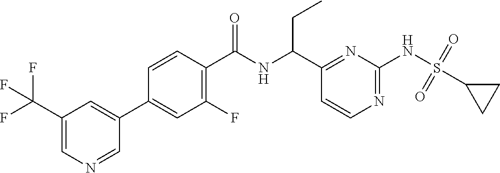 [SMILES: CCC(NC(=O)c1ccc(-c2cncc(C(F)(F)F)c2)cc1F)c1ccnc(NS(=O)(=O)C2CC2)n1] |
| P212 | 4-(5-chloropyridin-3-yl)-N-(1-(2- |
| | (cyclopropanesulfonamido)pyrimidin-4- |
| | yl)propyl)-2-fluorobenzamide |
| | 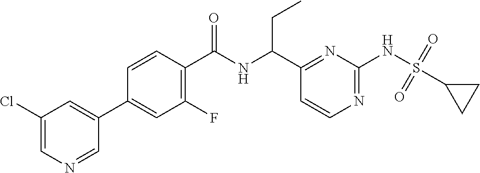 [SMILES: CCC(NC(=O)c1ccc(-c2cncc(Cl)c2)cc1F)c1ccnc(NS(=O)(=O)C2CC2)n1] |
| P213 | N-(1-(2- |
| | (cyclopropanesulfonamido)pyrimidin-4- |
| | yl)propyl)-4-(5-(trifluoromethyl)pyridin-3- |
| | yl)benzamide |
| | 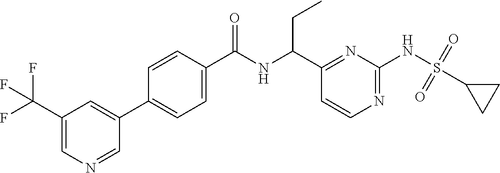 [SMILES: CCC(NC(=O)c1ccc(-c2cncc(C(F)(F)F)c2)cc1)c1ccnc(NS(=O)(=O)C2CC2)n1] |
| P214 | 4-(5-chloropyridin-3-yl)-N-(1-(2- |
| | (cyclopropanesulfonamido)pyrimidin-4- |
| | yl)propyl)benzamide |
| | 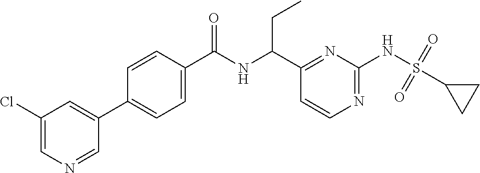 [SMILES: CCC(NC(=O)c1ccc(-c2cncc(Cl)c2)cc1)c1ccnc(NS(=O)(=O)C2CC2)n1] |
| P215 | N-(1-(2- |
| | (cyclopropanesulfonamido)pyrimidin-4- |
| | yl)propyl)-4-(6-ethoxypyrazin-2-yl)-2- |
| | (trifluoromethyl)benzamide |
| | 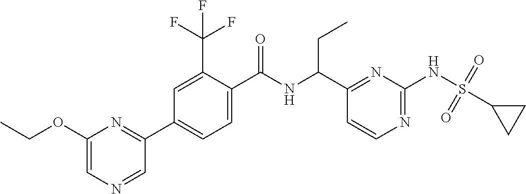 [SMILES: CCOc1cncc(-c2ccc(C(=O)NC(CC)c3ccnc(NS(=O)(=O)C4CC4)n3)c(C(F)(F)F)c2)n1] |
| P216 | N-(1-(2- |
| | (cyclopropanesulfonamido)pyrimidin-4- |
| | yl)propyl)-4-(6-ethoxypyrazin-2-yl)-2- |
| | fluorobenzamide |
| | 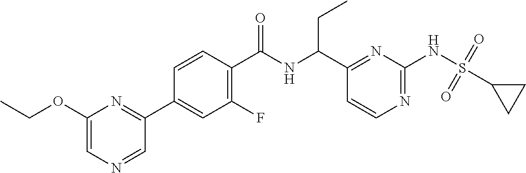 [SMILES: CCOc1cncc(-c2ccc(C(=O)NC(CC)c3ccnc(NS(=O)(=O)C4CC4)n3)c(F)c2)n1] |
| P217 | N-(1-(2- |
| | (cyclopropanesulfonamido)pyrimidin-4- |
| | yl)propyl)-4-(6-(trifluoromethyl)pyrazin-2- |
| | yl)benzamide |
| | 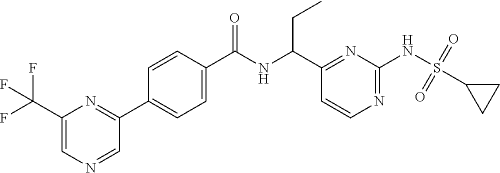 [SMILES: CCC(NC(=O)c1ccc(-c2cncc(C(F)(F)F)n2)cc1)c1ccnc(NS(=O)(=O)C2CC2)n1] |
| P218 | N-(1-(2- |
| | (cyclopropanesulfonamido)pyrimidin-4- |
| | yl)propyl)-4-(6-isopropoxypyrazin-2- |
| | yl)benzamide |
| | 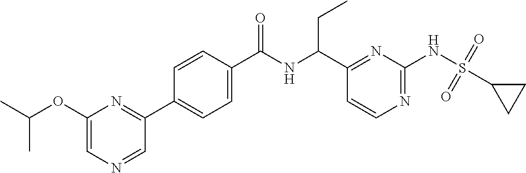 [SMILES: CCC(NC(=O)c1ccc(-c2cncc(OC(C)C)n2)cc1)c1ccnc(NS(=O)(=O)C2CC2)n1] |
| P219 | N-(1-(2- |
| | (cyclopropanesulfonamido)pyrimidin-4- |
| | yl)propyl)-4-(6-ethoxypyrazin-2- |
| | yl)benzamide |
| | 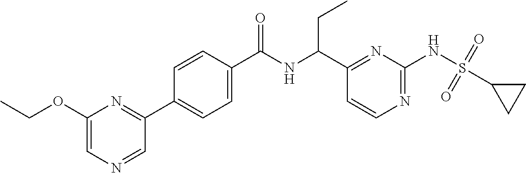 [SMILES: CCOc1cncc(-c2ccc(C(=O)NC(CC)c3ccnc(NS(=O)(=O)C4CC4)n3)cc2)n1] |
| P220 | N-(2-(2- |
| | (cyclopropanesulfonamido)pyrimidin-4- |
| | yl)butan-2-yl)-4-(6-ethoxypyrazin-2-yl)-2- |
| | fluorobenzamide |
| | 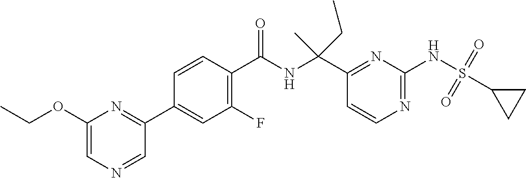 [SMILES: CCOc1cncc(-c2ccc(C(=O)NC(C)(CC)c3ccnc(NS(=O)(=O)C4CC4)n3)c(F)c2)n1] |
| P221 | N-(2-(6- |
| | (cyclopropanesulfonamido)pyrazin-2- |
| | yl)propan-2-yl)-2-fluoro-4-(6- |
| | isopropoxypyrazin-2-yl)benzamide |
| | 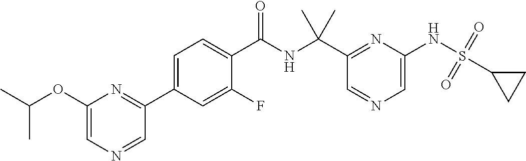 [SMILES: CC(C)Oc1cncc(-c2ccc(C(=O)NC(C)(C)c3cncc(NS(=O)(=O)C4CC4)n3)c(F)c2)n1] |
| P222 | N-(2-(6- |
| | (cyclopropanesulfonamido)pyrazin-2- |
| | yl)propan-2-yl)-4-(6- |
| | (trifluoromethyl)pyrazin-2-yl)benzamide |
| | 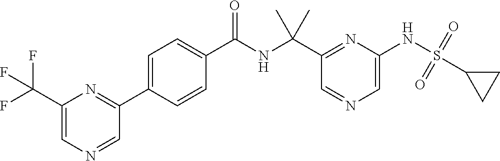 [SMILES: CC(C)(NC(=O)c1ccc(-c2cncc(C(F)(F)F)n2)cc1)c1cncc(NS(=O)(=O)C2CC2)n1] |
| P223 | N-(1-(6- |
| | (cyclopropanesulfonamido)pyrazin-2- |
| | yl)propyl)-4-(6-ethoxypyrazin-2-yl)-2- |
| | fluorobenzamide |
| | 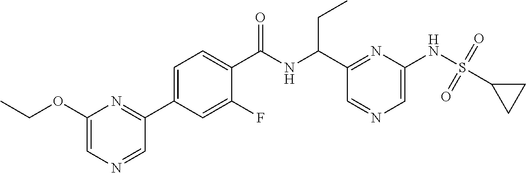 [SMILES: CCOc1cncc(-c2ccc(C(=O)NC(CC)c3cncc(NS(=O)(=O)C4CC4)n3)c(F)c2)n1] |
| P224 | Single enantiomer - stereochemistry |
| | unassigned |
| | N-(1-(6- |
| | (cyclopropanesulfonamido)pyrazin-2- |
| | yl)propyl)-4-(6-ethoxypyrazin-2-yl)-2- |
| | fluorobenzamide |
| | 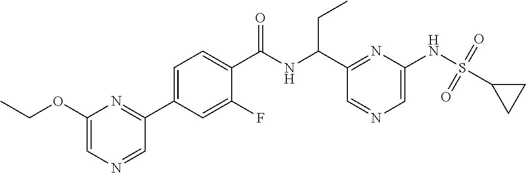 [SMILES: CCOc1cncc(-c2ccc(C(=O)NC(CC)c3cncc(NS(=O)(=O)C4CC4)n3)c(F)c2)n1] |
| P225 | Single enantiomer - stereochemistry |
| | unassigned |
| | N-(1-(6- |
| | (cyclopropanesulfonamido)pyrazin-2- |
| | yl)propyl)-4-(6-ethoxypyrazin-2-yl)-2- |
| | fluorobenzamide |
| | 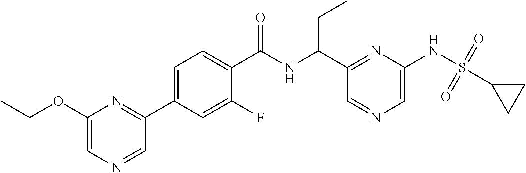 [SMILES: CCOc1cncc(-c2ccc(C(=O)NC(CC)c3cncc(NS(=O)(=O)C4CC4)n3)c(F)c2)n1] |

### 2-(2-(Cydopropanesulfonamido)pyrimidin-4-yl)-N-(5-(6-isopropylpyrazin-2-yl)pyridin-2-yl)-2-methylpropanamide P116

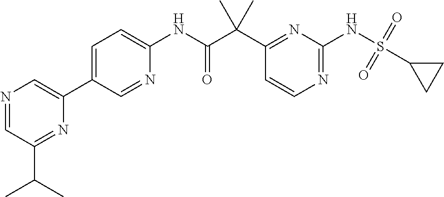 [SMILES: CC(C)c1cncc(-c2ccc(NC(=O)C(C)(C)c3ccnc(NS(=O)(=O)C4CC4)n3)nc2)n1]

A solution of 2-(2-(cydopropanesulfonamido)pyrimidin-4-yl)-2-methyl-N-(5-(6-(prop-1-en-2-yl)pyrazin-2-yl)pyridin-2-yl)propanamide P122 (77 mg, 0.161 mmol) in MeOH/DCM (4:1, 10 mL) was hydrogenated using the H-Cube flow hydrogenation apparatus (10% Pd/C, 30×4 mm, Full hydrogen, 25° C., 1 mL/min). The crude product was purified by chromatography on silica gel (12 g column, 50-100% EtOAc/iso-hexane) to afford 2-(2-(cyclopropanesulfonamido)pyrimidin-4-yl)-N-(5-(6-isopropylpyrazin-2-yl)pyridin-2-yl)-2-methylpropanamide (21 mg, 0.043 mmol, 27% yield) as a white solid. Rt 2.22 mins (HPLC acidic); m/z 482 (M+H)⁺ (ES⁺); ¹H NMR (500 MHz, DMSO-d6) δ 11.23 (s, 1H), 10.15 (s, 1H), 9.10 (s, 1H), 9.03 (dd, J=2.4, 0.8 Hz, 1H), 8.59 (d, J=5.3 Hz, 1H), 8.56 (s, 1H), 8.52 (dd, J=8.8, 2.5 Hz, 1H), 8.21 (dd, J=8.8, 0.8 Hz, 1H), 7.19 (d, J=5.3 Hz, 1H), 3.23-3.10 (m, 2H), 1.61 (s, 6H), 1.32 (d, J=6.9 Hz, 6H), 1.04-0.97 (m, 2H), 0.80-0.72 (m, 2H).

The following compounds were prepared using the methods described herein and below. Numbering of certain intermediates refers either to intermediates disclosed herein, or intermediates disclosed in WO02019/179852, WO2019/180244 and/or WO2020/083975.

### 1-(2-(Cydopropanesulfonamido)pyrimidin-4-yl)-N-(5-(6-ethoxypyrazin-2-yl)pyridin-2-yl)-4-oxocyclohexanecarboxamide P226

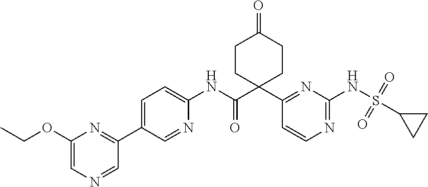 [SMILES: CCOc1cncc(-c2ccc(NC(=O)C3(c4ccnc(NS(=O)(=O)C5CC5)n4)CCC(=O)CC3)nc2)n1]

A solution of HCl (1N in water) (17.19 mL, 17.19 mmol) was added into a stirring solution of 8-(2-(cydopropanesulfonamido)pyrimidin-4-yl)-N-(5-(6-ethoxypyrazin-2-yl)pyridin-2-yl)-1,4-dioxaspiro[4.5]decane-8-carboxamide P244 (1.0 g, 1.72 mmol) in THF (30 mL). The resulting reaction mixture was stirred at 30° C. for 14 days. The reaction mixture was diluted with EtOAc (200 mL) and washed with water (100 mL) and brine (100 mL). The organic extract was dried (MgSO₄), filtered and concentrated in vacuo. The crude product was purified by chromatography on silica gel (40 g cartridge, 0-100% EtOAc/iso-hexanes) to afford 1-(2-(cydopropanesulfonamido)pyrimidin-4-yl)-N-(5-(6-ethoxypyrazin-2-yl)pyridin-2-yl)-4-oxocyclohexanecarboxamide (414 mg, 0.762 mmol, 44% yield) as a white solid. Rt 2.03 min (HPLC acidic); m/z 538 (M+H)⁺(ES⁺). ¹H NMR (500 MHz, DMSO-d6) δ 11.32 (s, 1H), 10.33 (s, 1H), 9.03 (d, J=2.5 Hz, 1H), 8.85 (s, 1H), 8.64 (d, J=5.3 Hz, 1H), 8.51 (dd, J=8.8, 2.5 Hz, 1H), 8.31-8.20 (m, 2H), 7.30 (d, J=5.3 Hz, 1H), 4.48 (q, J=7.0 Hz, 2H), 3.28-3.21 (m, 1H), 2.78-2.68 (m, 2H), 2.60-2.41 (m, 4H (obscured by DMSO)), 2.39-2.32 (m, 2H), 1.40 (t, J=7.0 Hz, 3H), 110-1.02 (m, 2H), 0.92-0.82 (m, 2H).

### 1-(2-(Cyclopropanesulfonamido)pyrimidin-4-yl)-N-(5-(6-ethoxypyrazin-2-yl)pyridin-2-yl)-4-hydroxycyclohexane-1-carboxamide

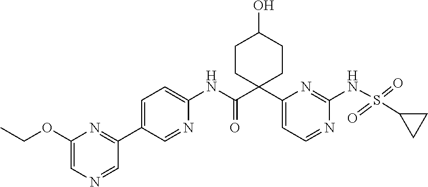 [SMILES: CCOc1cncc(-c2ccc(NC(=O)C3(c4ccnc(NS(=O)(=O)C5CC5)n4)CCC(O)CC3)nc2)n1]

NaBH₄ (10.6 mg, 0.28 mmol) was added into a stirring suspension of 1-(2-(cydopropanesulfonamido)pyrimidin-4-yl)-N-(5-(6-ethoxypyrazin-2-yl)pyridin-2-yl)-4-oxocyclohexanecarboxamide P226 (100 mg, 0.186 mmol) in EtOH (20 mL) and stirred at RT for 3 hrs. The reaction mixture was concentrated in vacuo and the crude product was purified by chromatography on RP Flash C18 (24 g column, 0-100% MeCN/Water 0.1% formic acid) to afford two diastereoisomers of the title compound.

P227—First Eluting Peak from Column

1-(2-(cydopropanesulfonamido)pyrimidin-4-yl)-N-(5-(6-ethoxypyrazin-2-yl)pyridin-2-yl)-4-hydroxycyclohexanecarboxamide (26 mg, 0.048 mmol, 26% yield) as a white solid. Rt 1.85 min (HPLC, acidic); m/z 540 (M+H)⁺ (ES⁺); ¹H NMR (500 MHz, DMSO-d6) δ 11.24 (s, 1H), 10.05 (s, 1H), 9.06-8.98 (m, 1H), 8.85 (s, 1H), 8.57 (d, J=5.3 Hz, 1H), 8.51 (dd, J=8.8, 2.5 Hz, 1H), 8.26 (s, 1H), 8.23-8.20 (m, 1H), 7.19 (d, J=5.3 Hz, 1H), 4.58 (d, J=4.7 Hz, 1H), 4.48 (q, J=7.0 Hz, 2H), 3.56-3.45 (m, 1H), 3.29-3.22 (m, 1H), 2.60 (d, J=13.6 Hz, 2H), 1.91-1.77 (m, 4H), 1.52-1.37 (m, 5H), 1.07-0.99 (m, 2H), 0.88-0.78 (m, 2H).

P228—Second Eluting Peak from Column

1-(2-(cyclopropanesulfonamido)pyrimidin-4-yl)-N-(5-(6-ethoxypyrazin-2-yl)pyridin-2-yl)-4-hydroxycyclohexanecarboxamide (23 mg, 0.042 mmol, 22% yield) as a white solid. Rt 1.95 min (HPLC, acidic); m/z 540 (M+H)⁺(ES⁺); ¹H NMR (500 MHz, DMSO-d6) δ 11.31 (s, 1H), 9.75 (s, 1H), 9.02 (d, J=2.4 Hz, 1H), 8.83 (s, 1H), 8.62 (s, 1H), 8.48 (dd, J=8.8, 2.5 Hz, 1H), 8.25 (s, 1H), 8.13 (d, J=8.8 Hz, 1H), 7.29 (s, 1H), 4.51-4.44 (m, 3H), 3.70-3.62 (m, 1H), 3.29-3.20 (m, 1H), 2.22-2.11 (m, 2H), 1.80-1.68 (m, 2H), 1.41-1.29 (m, 7H), 1.11-1.05 (m, 2H), 0.96-0.88 (m, 2H).

### 1-(2-(Cydopropanesulfonamido)pyrimidin-4-yl)-4-(dimethylamino)-N-(5-(6-ethoxypyrazin-2-yl)pyridin-2-yl)cyclohexane-1-carboxamide

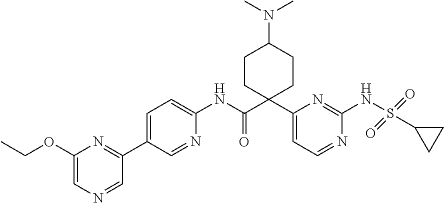 [SMILES: CCOc1cncc(-c2ccc(NC(=O)C3(c4ccnc(NS(=O)(=O)C5CC5)n4)CCC(N(C)C)CC3)nc2)n1]

NaH(BOAc)₃ (118 mg, 0.558 mmol) was added into a suspension of 1-(2-(cydopropanesulfonamido)pyrimidin-4-yl)-N-(5-(6-ethoxypyrazin-2-yl)pyridin-2-yl)-4-oxocyclohexanecarboxamide (100 mg, 0.186 mmol) P226 and dimethylamine (2M in THF) (0.93 mL, 1.86 mmol) in DCM (10 mL) and the resulting reaction mixture was stirred at RT for 18 hrs. The reaction mixture was concentrated in vacuo and the crude product was purified by preparative HPLC (Acidic prep method (5-95% MeCN in water) to afford two diastereoisomers of the title compound.

P229—First Eluting Peak from Prep HPLC

1-(2-(cyclopropanesulfonamido)pyrimidin-4-yl)-4-(dimethylamino)-N-(5-(6-ethoxypyrazin-2-yl)pyridin-2-yl)cyclohexane-1-carboxamide (22 mg, 0.037 mmol, 20% yield) as a white solid. Rt 1.40 min (HPLC, acidic); m/z 567 (M+H)⁺(ES⁺); ¹H NMR (500 MHz, DMSO-d6) δ 10.17 (s, 1H), 9.02 (d, J=2.4 Hz, 1H), 8.84 (s, 1H), 8.60-8.44 (m, 2H), 8.25 (s, 1H), 8.22-8.20 (m, 2H), 7.09 (d, J=5.3 Hz, 1H), 4.48 (q, J=7.0 Hz, 2H), 3.28-3.20 (m, 1H), 2.72 (d, J=13.4 Hz, 2H), 2.32 (s, 6H), 2.25 (s, 1H), 1.91 (d, J=12.3 Hz, 2H), 1.79 (t, J=12.6 Hz, 2H), 1.51 (q, J=12.2 Hz, 2H), 1.40 (t, J=7.0 Hz, 3H), 1.03-0.98 (m, 2H), 0.87-0.73 (m, 2H).

P230—Second Eluting Peak from Prep HPLC

1-(2-(cyclopropanesulfonamido)pyrimidin-4-yl)-4-(dimethylamino)-N-(5-(6-ethoxypyrazin-2-yl)pyridin-2-yl)cyclohexane-1-carboxamide (26 mg, 0.045 mmol, 24% yield) as a white solid. Rt 1.48 min (HPLC, acidic); m/z 567 (M+H)⁺(ES⁺); ¹H NMR (500 MHz, DMSO-d6) δ 9.65 (s, 1H), 9.02 (d, J=2.4 Hz, 1H), 8.83 (s, 1H), 8.55 (d, J=5.2 Hz, 1H), 8.48 (dd, J=8.8, 2.4 Hz, 1H), 8.25 (s, 1H), 8.20 (s, 1H), 8.10 (d, J=8.8 Hz, 1H), 7.19 (d, J=5.3 Hz, 1H), 4.47 (q, J=7.0 Hz, 2H), 3.25-3.18 (m, 1H), 2.78-2.67 (m, 2H), 2.48-2.39 (m, 1H), 2.26 (s, 6H), 2.02 (t, J=12.4 Hz, 2H), 1.87-1.77 (m, 2H), 1.42-1.29 (m, 5H), 1.07-1.00 (m, 2H), 0.97-0.85 (m, 2H).

Method 11: i-PrMgCl mediated amide coupling from ester

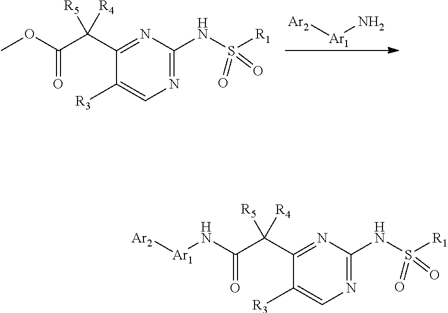

To an ice cooled solution of aniline (1.1 eq) in THF (10-50 volumes) was added i-PrMgCl (2.0 M in THF, 2.0 eq) dropwise over 5-15 mins to maintain an internal temperature of less than 10° C. The reaction mixture was warmed to RT over 45 mins, then a solution of ester (1.0 eq) in THF (5-20 volumes) was added dropwise over 5-15 min. The reaction mixture was stirred at ambient temperature for 5-15 mins then further i-PrMgCl (2.0 M in THF, 2.0 eq) was added dropwise over 5-20 min. The reaction mixture was stirred at RT for 30 mins and then the solution was slowly poured into 1M HCl (aq) and extracted with EtOAc. The organics were combined, dried (phase separator) and concentrated in vacuo. The crude product was purified by reverse or normal phase chromatography or a combination of both.

Reductive Amination General Method:

Method 12: Reductive Amination

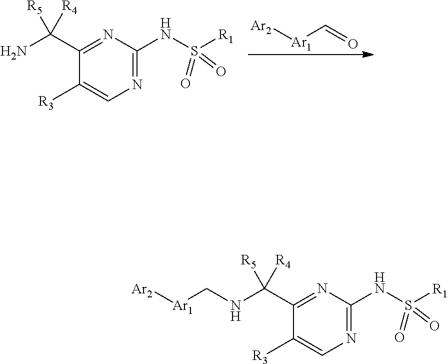

To a suspension of aldehyde (1.5 eq) and amine (HCl salt can be used, 1.0 eq) in an organic solvent such as DCM (2-10 volumes) was added AcOH (1.0 eq) at RT and stirred for up to 1 hr. NaBH(OAc)₃ (1-2 eq) was then added and stirring continued for up to 24 hrs and monitored by LCMS. On completion 1% NH₃ in MeOH (10 volumes) was added and the volatiles removed in vacuo. The crude product was purified by reverse or normal phase chromatography or a combination of both.

### N-(4-(1-((4-(6-Ethoxypyrazin-2-yl)-2-fluorobenzyl)amino)propyl)pyrimidin-2-yl)cyclopropanesulfonamide P235

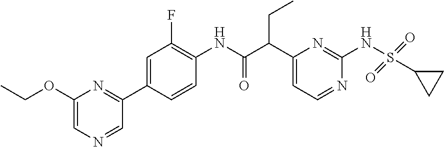 [SMILES: CCOc1cncc(-c2ccc(NC(=O)C(CC)c3ccnc(NS(=O)(=O)C4CC4)n3)c(F)c2)n1]

A suspension of 4-(6-ethoxypyrazin-2-yl)-2-fluorobenzaldehyde INTD88 (259 mg, 1.05 mmol) and N-(4-(1-aminopropyl)pyrimidin-2-yl)cyclopropanesulfonamide INTC162 (300 mg, 1.05 mmol) in DCM (2 mL) was treated with AcOH (0.065 mL, 1.14 mmol) and stirred for 15 mins then NaBH(OAc)₃ (223 mg, 1.06 mmol) was added and the reaction mixture was stirred at RT for 3 hrs. To the reaction mixture was added 1% NH₃ in MeOH (2 mL) and the volatiles were removed in vacuo. The crude product was purified by chromatography on RP Flash C18 (12 g cartridge, 15-70% MeCN/10 mM ammonium bicarbonate). The crude material was purified by capture and release on SCX (1 g) eluting with MeOH (20 mL) then removing product with 1% NH₃ in MeOH (30 mL). The crude material was finally purified a second time by chromatography on RP Flash C18 (12 g cartridge, 10-50% MeCN/10 mM Ammonium Bicarbonate) to afford N-(4-(1-((4-(6-ethoxypyrazin-2-yl)-2-fluorobenzyl)amino)propyl)pyrimidin-2-yl)cyclopropanesulfonamide (20 mg, 0.031 mmol, 3% yield) as a yellow gum. Rt 2.02 min (HPLC, basic); m/z 487 (M+H)⁺(ES⁺); ¹H NMR (500 MHz, DMSO-d6) δ 8.85 (s, 1H), 8.27-8.26 (m, 2H), 7.96 (d, J=7.8 Hz, 1H), 7.87 (d, J=11.4 Hz, 1H), 7.67-7.58 (m, 1H), 7.23 (d, J=5.1 Hz, 1H), 5.38 (t, J=5.7 Hz, 1H), 4.62 (d, J=5.8 Hz, 2H), 4.49 (q, J=7.1 Hz, 2H), 3.52-3.50 (m, 1H), 1.71-1.67 (m, 2H), 1.41 (t, J=7.0 Hz, 3H), 1.13-1.09 (m, 2H), 1.04-1.01 (m, 2H), 0.84 (t, J=7.4 Hz, 3H). Two exchangeable protons not observed.

TABLE 5
Preparation methods and characterisation data of certain intermediates and examples P242 onwards

| | | Synthesis Method, | |
|---|---|---|---|
| | | [LCMS Method], | ¹H NMR Chemical Shift |
| INTC# | | m/z (M + H)⁺, | Data (DMSO-d6 unless |
| or P# | Name/Structure (All examples containing chiral centres are racemates unless stated) | (Rt/Min) | stated) |
| INTC 246 | 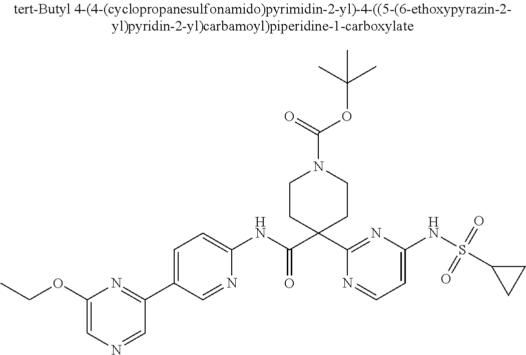 [SMILES: CCOc1cncc(-c2ccc(NC(=O)C3(c4nccc(NS(=O)(=O)C5CC5)n4)CCN(C(=O)OC(C)(C)C)CC3)nc2)n1] | Method 11 using INTC243 and INTD33, [UPLC Acidic], 625, (1.66) | 11.32 (s, 1H), 9.99 (s, 1H), 9.01 (d, J = 2.4 Hz, 1H), 8.84 (s, 1H), 8.58-8.55 (m, 1H), 8.53-8.47 (m, 1H), 8.25 (s, 1H), 8.21 (d, J = 8.8 Hz, 1H), 6.85 (d, J = 5.8 Hz, 1H), 4.48 (q, J = 7.0 Hz, 2H), 3.51-3.48 (m, 2H), 3.38-3.32 (m, 2H), 3.16- 3.12 (m, 1H), 2.32-2.29 (m, 4H), 1.41 (s, 9H), 1.40 (t, J = 7.10 Hz, 3H), 1.08 (s, 2H), 0.96-0.91 (m, 2H). |
| INTC 239 | 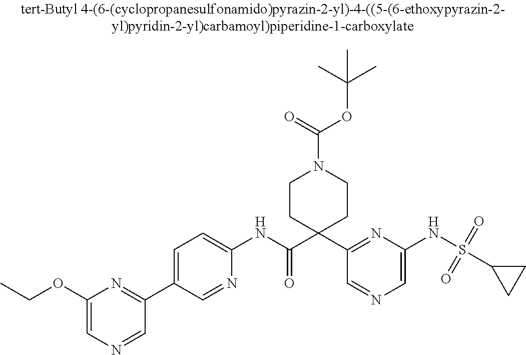 [SMILES: CCOc1cncc(-c2ccc(NC(=O)C3(c4cncc(NS(=O)(=O)C5CC5)n4)CCN(C(=O)OC(C)(C)C)CC3)nc2)n1] | Method 11 using INTC238 and INTD33, [UPLC Acidic], 625, (1.63) | 11.06 (s, 1H), 10.17 (s, 1H), 9.01 (s, 1H), 8.83 (s, 1H), 8.52-8.46 (m, 1H), 8.42 (s, 1H), 8.25 (s, 1H), 8.21-8.15 (m, 2H), 4.47 (q, J = 7.0 Hz, 2H), 3.71-3.65 (m, 2H), 3.28-3.16 (m, 2H), 3.13-3.03 (m, 1H), 2.18-2.05 (m, 2H), 1.43-1.36 (m, 12H), 1.05-1.02 (m, 2H), 0.86-0.81 (m, 2H). 2H not observed, obscured by DMSO peak. |
| INTC 244 | 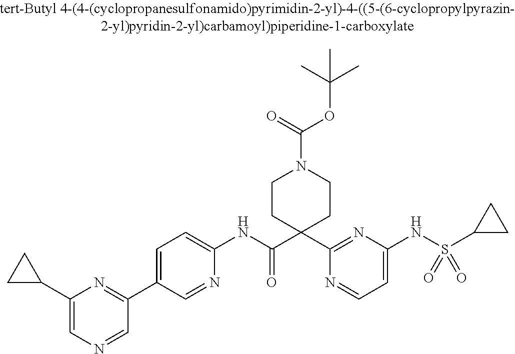 [SMILES: CC(C)(C)OC(=O)N1CCC(C(=O)Nc2ccc(-c3cncc(C4CC4)n3)cn2)(c2nccc(NS(=O)(=O)C3CC3)n2)CC1] | Method 11 using INTC243 and INTD54, [HPLC Acidic], 621, (2.57) | 11.32 (s, 1H), 9.97 (s, 1H), 9.02-8.95 (m, 2H), 8.58 (s, 1H), 8.56 (s, 1H), 8.46 (dd, J = 8.8, 2.4 Hz, 1H), 8.19 (d, J = 8.8 Hz, 1H), 6.85 (d, J = 5.8 Hz, 1H), 3.50-3.47 (m, 2H), 3.42-3.26 (m, 2H), 3.16-3.12 (m, 1H), 2.32-2.22 (m, 5H), 1.41 (s, 9H), 1.17-1.06 (m, 6H), 0.96-0.91 (m, 2H). |
| P242 | 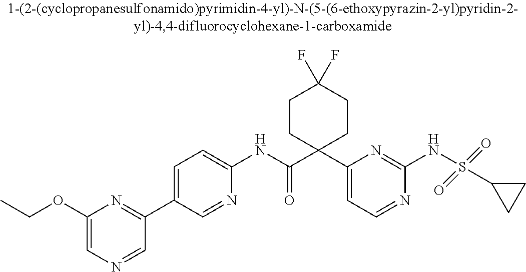 [SMILES: CCOc1cncc(-c2ccc(NC(=O)C3(c4ccnc(NS(=O)(=O)C5CC5)n4)CCC(F)(F)CC3)nc2)n1] | Method 11: using INTC212 and INTD33, [HPLC Acidic], 560, (2.42) | 11.33 (s, 1H), 10.28 (s, 1H), 9.03 (dd, J = 2.5, 0.8 Hz, 1H), 8.85 (s, 1H), 8.63 (d, J = 5.3 Hz, 1H), 8.51 (dd, J = 8.8, 2.5 Hz, 1H), 8.26 (s, 1H), 8.19 (dd, J = 8.8, 0.8 Hz, 1H), 7.26 (d, J = 5.4 Hz, 1H), 4.48 (q, J = 7.1 Hz, 2H), 3.27-3.18 (m, 1H), 2.65-2.53 (m, 2H), 2.29-2.18 (m, 2H), 2.13-2.01 (m, 4H), 1.40 (t, J = 7.0 Hz, 3H), 1.11-1.02 (m, 2H), 0.92-0.83 (m, 2H). |
| P244 | 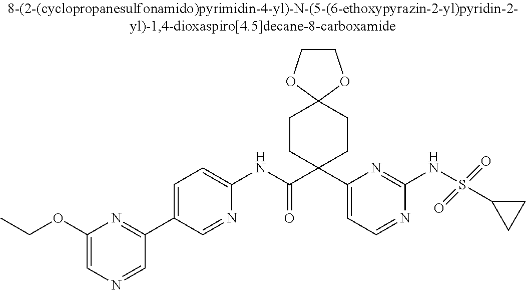 [SMILES: CCOc1cncc(-c2ccc(NC(=O)C3(c4ccnc(NS(=O)(=O)C5CC5)n4)CCC4(CC3)OCCO4)nc2)n1] | Method 11: using INTC214 and INTD33, [HPLC Acidic], 582, (2.20) | 11.29 (s, 1H), 10.05 (s, 1H), 9.02 (dd, J = 2.5, 0.8 Hz, 1H), 8.84 (s, 1H), 8.60 (d, J = 5.3 Hz, 1H), 8.50 (dd, J = 8.8, 2.5 Hz, 1H), 8.26 (s, 1H), 8.22-8.11 (m, 1H), 7.23 (s, 1H), 4.48 (q, J = 7.0 Hz, 2H), 3.88 (s, 4H), 3.28-3.18 (m, 1H), 2.55-2.47 (m, 2H obscured by water peak), 2.27-2.13 (m, 2H), 1.79-1.62 (m, 4H), 1.40 (t, J = 7.0 Hz, 3H), 1.09-1.00 (m, 2H), 0.94-0.80 (m, 2H). |
| P248 | 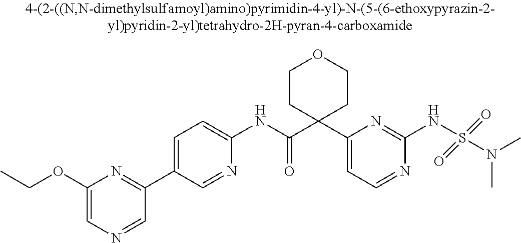 [SMILES: CCOc1cncc(-c2ccc(NC(=O)C3(c4ccnc(NS(=O)(=O)N(C)C)n4)CCOCC3)nc2)n1] | Method R: using INTC204, [HPLC Acidic], 529, (2.10) | 11.04 (s, 1H), 10.02 (s, 1H), 9.04 (d, J = 2.5 Hz, 1H), 8.85 (s, 1H), 8.60 (d, J = 5.3 Hz, 1H), 8.51 (dd, J = 8.8, 2.5 Hz, 1H), 8.26 (s, 1H), 8.19 (d, J = 8.8 Hz, 1H), 7.22 (d, J = 5.3 Hz, 1H), 4.48 (q, J = 7.0 Hz, 2H), 3.73-3.57 (m, 4H), 2.84 (s, 6H), 2.47-2.37 (m, 2H), 2.28-2.17 (m, 2H), 1.40 (t, J = 7.0 Hz, 3H). |
| P251 | 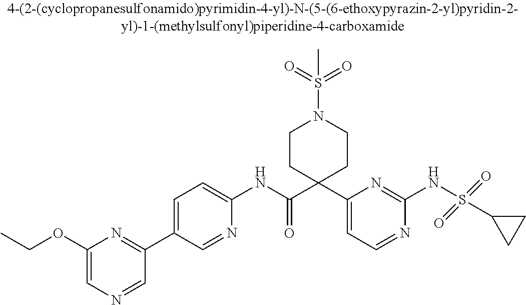 [SMILES: CCOc1cncc(-c2ccc(NC(=O)C3(c4ccnc(NS(=O)(=O)C5CC5)n4)CCN(S(C)(=O)=O)CC3)nc2)n1] | Method 11: using INTC215 and INTD33, [HPLC Acidic], 603, (2.05) | 11.33 (s, 1H), 10.27 (s, 1H), 9.03 (d, J = 2.4 Hz, 1H), 8.85 (s, 1H), 8.63 (d, J = 5.3 Hz, 1H), 8.54-8.48 (m, 1H), 8.26 (s, 1H), 8.21 (d, J = 8.8 Hz, 1H), 7.26 (d, J = 5.3 Hz, 1H), 4.48 (q, J = 7.0 Hz, 2H), 3.47-3.40 (m, 2H), 3.29-3.21 (m, 1H), 3.10 (t, J = 10.7 Hz, 2H), 2.87 (s, 2H), 2.64-2.57 (m, 2H), 2.33-2.21 (m, 2H), 1.43-1.37 (m, 3H), 1.32-1.09 (m, 1H), 1.09-1.04 (m, 2H), 0.92-0.80 (m, 2H). |
| P254 | 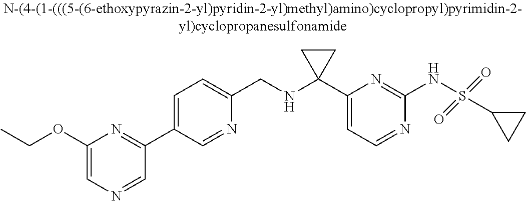 [SMILES: CCOc1cncc(-c2ccc(CNC3(c4ccnc(NS(=O)(=O)C5CC5)n4)CC3)nc2)n1] | Method 12 using INTC156 and INTD85, [UPLC Basic], 468, (1.07) | 11.08 (s, 1H), 9.22 (d, J = 2.3 Hz, 1H), 8.88 (s, 1H), 8.50-8.42 (m, 2H), 8.29 (s, 1H), 7.64 (d, J = 8.2 Hz, 1H), 7.43 (d, J = 5.2 Hz, 1H), 4.50 (q, J = 7.0 Hz, 2H), 3.94 (s, 2H), 3.16-3.13 (m, 1H), 1.41 (t, J = 7.0 Hz, 3H), 1.38-1.33 (m, 2H), 1.26-1.21 (m, 2H), 1.09-0.99 (m, 4H). One exchangeable proton not observed |
| P255 | 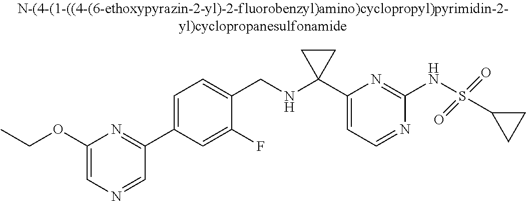 [SMILES: CCOc1cncc(-c2ccc(CNC3(c4ccnc(NS(=O)(=O)C5CC5)n4)CC3)c(F)c2)n1] | Method 12 using INTC156 and INTD88, [UPLC Basic], 485, (1.36) | 11.06 (s, 1H), 8.86 (d, J = 2.4 Hz, 1H), 8.48 (s, 1H), 8.27 (d, J = 2.5 Hz, 1H), 7.97 (d, J = 8.3 Hz, 1H), 7.90 (d, J = 11.5 Hz, 1H), 7.70-7.63 (m, 1H), 7.43 (s, 1H), 4.53-4.45 (m, 2H), 3.84 (s, 2H), 3.16-3.12 (m, 1H), 1.44-1.39 (m, 3H), 1.38-1.32 (m, 2H), 1.27-1.21 (m, 2H), 1.09-1.01 (m, 4H). One exchangeable proton not observed |
| P256 | 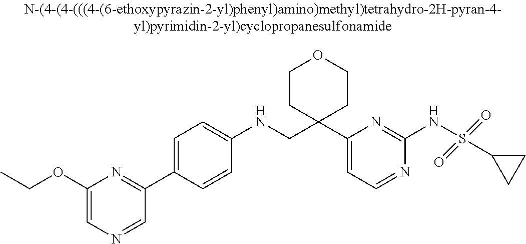 [SMILES: CCOc1cncc(-c2ccc(NCC3(c4ccnc(NS(=O)(=O)C5CC5)n4)CCOCC3)cc2)n1] | Method R using INTC207, [HPLC Acidic], 511, (2.19) | 11.23 (s, 1H), 8.57 (s, 1H), 8.51 (d, J = 5.3 Hz, 1H), 8.00 (s, 1H), 7.83-7.74 (m, 2H), 7.22 (d, J = 5.1 Hz, 1H), 6.67-6.59 (m, 2H), 6.02 (d, J = 6.5 Hz, 1H), 4.42 (q, J = 7.0 Hz, 2H), 3.80-3.69 (m, 2H), 3.42-3.27 (m, 4H), 3.25-3.17 (m, 1H), 2.34-2.25 (m, 2H), 1.89-1.80 (m, 2H), 1.38 (t, J = 7.0 Hz, 3H), 1.14-1.06 (m, 2H), 1.07-0.98 (m, 2H |
| P258 | 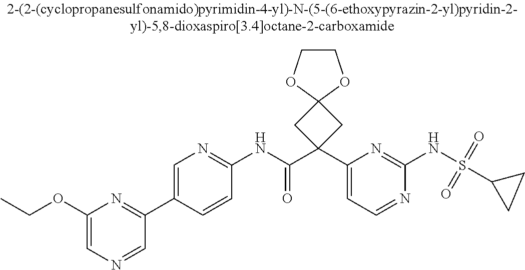 [SMILES: CCOc1cncc(-c2ccc(NC(=O)C3(c4ccnc(NS(=O)(=O)C5CC5)n4)CC4(C3)OCCO4)nc2)n1] | Method 11: using INTC216 and INTD33, [UPLC acidic], 554, (1.32) | 11.31 (s, 1H), 10.56 (s, 1H), 9.03 (d, J = 2.4 Hz, 1H), 8.84 (s, 1H), 8.60 (d, J = 5.3 Hz, 1H), 8.51 (dd, J = 8.8, 2.5 Hz, 1H), 8.26-8.20 (m, 2H), 7.30 (d, J = 5.3 Hz, 1H), 4.48 (q, J = 7.0 Hz, 2H), 3.89-3.78 (m, 4H), 3.32-3.25 (m, 1H), 3.18-3.11 (m, 2H), 2.99-2.92 (m, 2H), 1.39 (t, J = 7.0 Hz, 3H), 1.10-1.01 (m, 2H), 0.94-0.87 (m, 2H). |
| P260 | 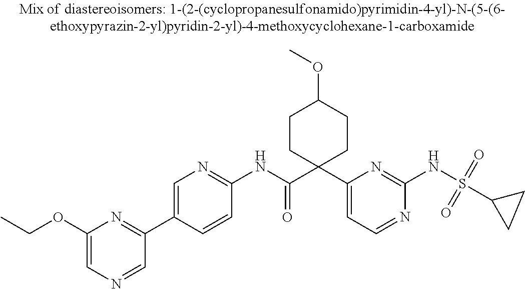 [SMILES: CCOc1cncc(-c2ccc(NC(=O)C3(c4ccnc(NS(=O)(=O)C5CC5)n4)CCC(OC)CC3)nc2)n1] | Method 11: using INTC218 and INTD33, [HPLC Acidic], 554, (2.17 and 2.27) | 11.26 (s, 1H), 10.04 (s, 1H), 9.02 (d, J = 2.4 Hz, 1H), 8.84 (d, J = 1.9 Hz, 1H), 8.60 (d, J = 5.6 Hz, 1H), 8.55-8.46 (m, 1H), 8.25 (s, 1H), 8.22-8.14 (m, 1H), 7.22 (d, J = 5.4 Hz, 1H), 4.48 (qd, J = 7.0, 2.3 Hz, 2H), 3.30-3.18 (m, 4H), 2.59 (d, J = 13.3 Hz, 2H), 2.40-2.19 (m, 1H), 1.97-1.84 (m, 2H), 1.83-1.50 (m, 2H), 1.48-1.36 (m, 4H), 1.11-0.98 (m, 2H), 0.94-0.71 (m, 3H). |
| P261 | 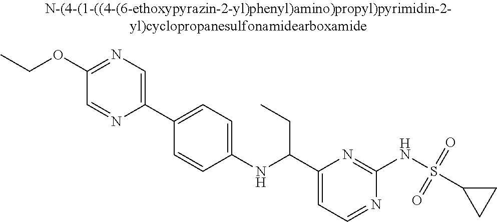 [SMILES: CCOc1cnc(-c2ccc(NC(CC)c3ccnc(NS(=O)(=O)C4CC4)n3)cc2)cn1] | Method 12 using INTC155 and INTD18, [UPLC Acidic], 455, (1.48) | Methanol-d4, 8.45-8.39 (m, 2H), 7.90 (s, 1H), 7.86-7.81 (m, 2H), 7.70-7.62 (m, 1H), 7.61-7.54 (m, 1H), 7.12 (d, J = 5.2 Hz, 1H), 6.69-6.64 (m, 2H), 4.49 (q, J = 7.1 Hz, 2H), 4.45-4.39 (m, 1H), 3.24-3.15 (m, 1H), 2.06-1.85 (m, 2H), 1.44 (t, J = 7.1 Hz, 3H), 1.34-1.25 (m, 1H), 1.24-1.15 (m, 1H), 1.09 (t, J = 7.4 Hz, 3H), 1.07-1.00 (m, 1H), 0.96-0.86 (m, 1H). |
| P288 | 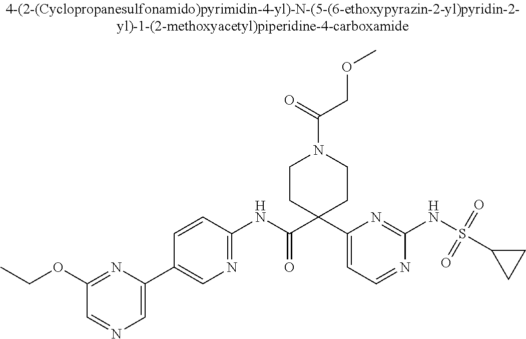 [SMILES: CCOc1cncc(-c2ccc(NC(=O)C3(c4ccnc(NS(=O)(=O)C5CC5)n4)CCN(C(=O)COC)CC3)nc2)n1] | Prepared by reacting P140 (in WO2019/179652) with 2- methoxyacetyl chloride, TEA, in DCM at RT for 24 h, [UPLC Acidic], 597, (1.21) | 11.38 (s, 1H), 10.21 (s, 1H), 9.02 (dd, J = 2.5, 0.8 Hz, 1H), 8.83 (s, 1H), 8.61 (d, J = 5.4 Hz, 1H), 8.49 (dd, J = 8.8, 2.5 Hz, 1H), 8.25 (s, 1H), 8.21-8.13 (m, 1H), 7.25-7.20 (m, 1H), 4.47 (q, J = 7.0 Hz, 2H), 4.10 (m, 2H), 3.90 (d, J = 13.5 Hz, 1H), 3.60 (d, J = 13.6 Hz, 1H), 3.28 (s, 3H), 3.26-3.21 (dt, m, 1H), 2.47 (s, 3H), 2.20-2.05 (m, 3H), 1.39 (t, J = 7.0 Hz, 3H), 1.05-1.03 (m, 2H), 0.91-0.85 (m, 2H). |
| P289 | 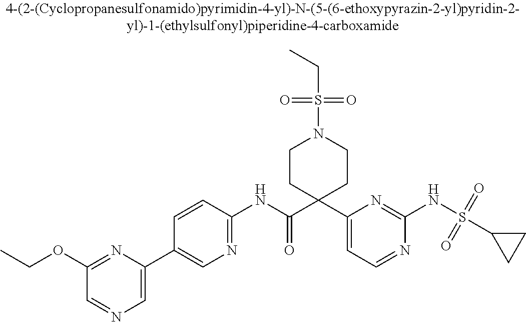 [SMILES: CCOc1cncc(-c2ccc(NC(=O)C3(c4ccnc(NS(=O)(=O)C5CC5)n4)CCN(S(=O)(=O)CC)CC3)nc2)n1] | Prepared by reacting P140 (in WO2019/179652) with ethanesulfonyl chloride, TEA, in DCM at RT for 24 h, [UPLC Acidic], 617, (1.38) | 11.32 (s, 1H), 10.25 (s, 1H), 9.02 (d, J = 2.5 Hz, 1H), 8.84 (s, 1H), 8.62 (d, J = 5.4 Hz, 1H), 8.50 (dd, J = 8.8, 2.5 Hz, 1H), 8.25 (s, 1H), 8.20 (d, J = 8.8 Hz, 1H), 7.26-7.22 (m, 1H), 4.47 (q, J = 7.0 Hz, 2H), 3.51-3.45 (m, 2H), 3.21-3.13 (m, 2H), 3.05 (q, J = 7.4 Hz, 2H), 2.61-2.54 (m, 2H), 2.26-2.18 (m, 1H), 1.39 (t, J = 7.0 Hz, 3H), 1.18 (t, J = 7.3 Hz, 3H), 1.08-1.02 (m, 2H), 0.91-0.80 (m, 4H). |
| P290 | 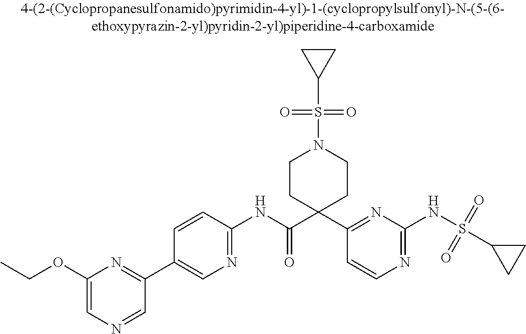 [SMILES: CCOc1cncc(-c2ccc(NC(=O)C3(c4ccnc(NS(=O)(=O)C5CC5)n4)CCN(S(=O)(=O)C4CC4)CC3)nc2)n1] | Prepared by reacting P140 (in WO2019/179652) with cyclopropane sulfonyl chloride, TEA, in DCM at RT for 24 h, [UPLC Acidic], 629, (1.38) | 11.32 (s, 1H), 10.29 (s, 1H), 9.03 (dd, J = 2.4, 0.8 Hz, 1H), 8.85 (s, 1H), 8.63 (d, J = 5.3 Hz, 1H), 8.51 (dd, J = 8.8, 2.5 Hz, 1H), 8.26 (s, 1H), 8.21 (dd, J = 8.8, 0.8 Hz, 1H), 7.26 (d, J = 5.4 Hz, 1H), 4.48 (q, J = 7.0 Hz, 2H), 3.55-3.49 (m, 2H), 3.29-3.11 (m, 3H), 2.69-2.52 (m, 4H), 2.29-2.17 (m, 2H), 1.40 (t, J = 7.0 Hz, 3H), 1.10-1.01 (m, 2H), 1.01-0.79 (m, 5H). |
| P291 | 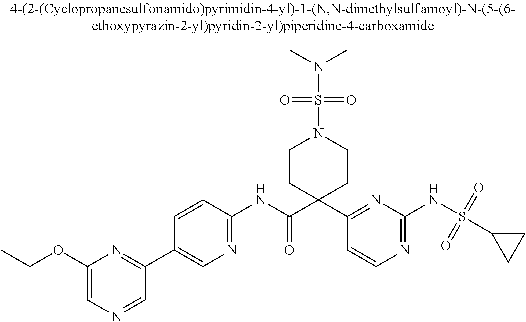 [SMILES: CCOc1cncc(-c2ccc(NC(=O)C3(c4ccnc(NS(=O)(=O)C5CC5)n4)CCN(S(=O)(=O)N(C)C)CC3)nc2)n1] | Prepared by reacting P140 (in WO2019/179652) with dimethylsulfamoyl chloride, TEA, in DCM at RT for 24 h, [UPLC Acidic], 632, (1.45) | 11.32 (s, 1H), 10.24 (s, 1H), 9.02 (d, J = 2.4 Hz, 1H), 8.84 (s, 1H), 8.62 (d, J = 5.3 Hz, 1H), 8.50 (dd, J = 8.8, 2.4 Hz, 1H), 8.25 (s, 1H), 8.19 (d, J = 8.7 Hz, 1H), 7.24 (d, J = 5.4 Hz, 1H), 4.47 (q, J = 7.0 Hz, 2H), 3.45-3.39 (m, 2H), 3.26-3.21 (m, 1H), 3.20-3.12 (m, 2H), 2.74 (s, 6H), 2.59-2.53 (m, 2H), 2.26-2.18 (m, 2H), 1.39 (t, J = 7.0 Hz, 3H), 1.07-1.04 (m, 2H), 0.86-0.82 (m, 2H). |
| P292 | 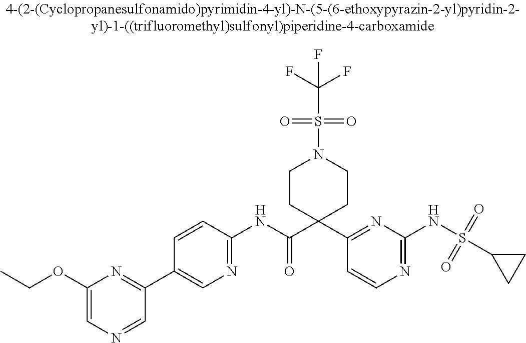 [SMILES: CCOc1cncc(-c2ccc(NC(=O)C3(c4ccnc(NS(=O)(=O)C5CC5)n4)CCN(S(=O)(=O)C(F)(F)F)CC3)nc2)n1] | Prepared by reacting P140 (in WO2019/179652) with trifluoromethane- sulfonic anhydride, TEA, in DCM at RT for 24 h, [UPLC Acidic], 657, (1.62) | 11.35 (s, 1H), 10.36 (s, 1H), 9.03 (d, J = 2.4 Hz, 1H), 8.84 (s, 1H), 8.62 (d, J = 5.3 Hz, 1H), 8.51 (dd, J = 8.8, 2.5 Hz, 1H), 8.25 (s, 1H), 8.19 (d, J = 8.8 Hz, 1H), 7.22 (d, J = 5.3 Hz, 1H), 4.47 (q, J = 7.0 Hz, 2H), 3.78-3.74 (m, 2H), 3.53-3.49 (m, 2H), 3.26-3.18 (m, 1H), 2.69-2.55 (m, 2H), 2.29 (s, 2H), 1.39 (t, J = 7.0 Hz, 3H), 1.08-1.03 (m, 2H), 0.92-0.77 (m, 2H). |
| P293 | 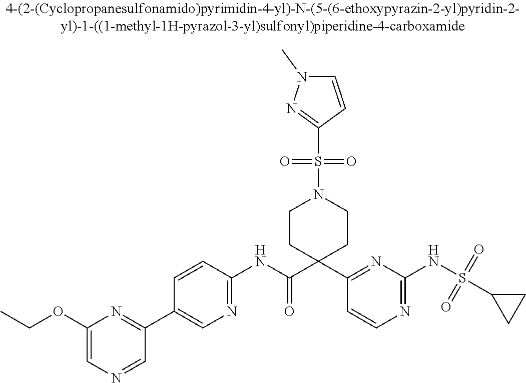 [SMILES: CCOc1cncc(-c2ccc(NC(=O)C3(c4ccnc(NS(=O)(=O)C5CC5)n4)CCN(S(=O)(=O)c4ccn(C)n4)CC3)nc2)n1] | Prepared by reacting P140 (in WO2019/179652) with methyl-1H- pyrazole-3- sulfonyl chloride, TEA, in DCM at RT for 24 h, [UPLC Acidic], 669, (1.34) | 11.30 (s, 1H), 10.23 (s, 1H), 9.01 (d, J = 2.5 Hz, 1H), 8.83 (s, 1H), 8.61 (d, J = 5.3 Hz, 1H), 8.47 (dd, J = 8.8, 2.5 Hz, 1H), 8.25 (s, 1H), 8.06 (d, J = 8.8 Hz, 1H), 7.90 (d, J = 2.3 Hz, 1H), 7.20 (d, J = 5.3 Hz, 1H), 6.65 (d, J = 2.3 Hz, 1H), 4.47 (q, J = 7.0 Hz, 2H), 3.88 (s, 3H), 3.58-3.38 (m, 2H), 3.25-3.16 (m, 1H), 2.92-2.85 (m, 2H), 2.62-2.56 (m, 2H), 2.25-2.16 (m, 2H), 1.39 (t, J = 7.0 Hz, 3H), 1.07-1.00 (m, 2H), 0.91-0.79 (m, 2H). |
| P294 | 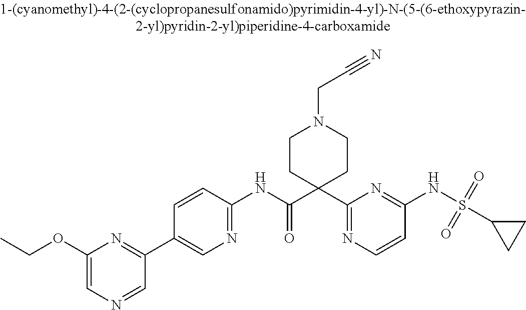 [SMILES: CCOc1cncc(-c2ccc(NC(=O)C3(c4nccc(NS(=O)(=O)C5CC5)n4)CCN(CC#N)CC3)nc2)n1] | Alkylation capping using INTC247 and 2- bromoacetonitrile, [UPLC Acidic], 564, (1.23) | 11.34 (s, 1H), 9.92 (s, 1H), 9.04-9.00 (m, 1H), 8.84 (s, 1H), 8.57 (s, 1H), 8.51 (dd, J = 8.8, 2.5 Hz, 1H), 8.25 (s, 1H), 8.21 (d, J = 8.7 Hz, 1H), 6.84 (s, 1H), 4.48 (q, J = 7.0 Hz, 2H), 3.70 (s, 2H), 3.18-3.14 (m, 1H), 2.42 (s, 6H), 1.40 (t, J = 7.0 Hz, 3H), 1.09-1.05 (m, 2H), 0.96-0.91 (m, 2H). 2H obscured in DMSO peak |
| P295 | 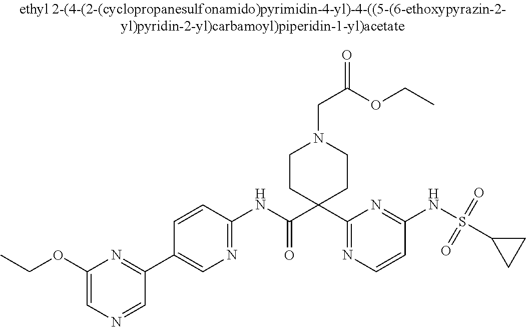 [SMILES: CCOC(=O)CN1CCC(C(=O)Nc2ccc(-c3cncc(OCC)n3)cn2)(c2nccc(NS(=O)(=O)C3CC3)n2)CC1] | Prepared by reacting INTC247 and ethyl 2- oxoacetate, [UPLC Acidic], 611, (0.95) | 11.32 (s, 1H), 9.83 (s, 1H), 9.01 (d, J = 2.5 Hz, 1H), 8.84 (s, 1H), 8.50 (dd, J = 8.8, 2.5 Hz, 1H), 8.25 (s, 1H), 8.20 (d, J = 8.8 Hz, 1H), 6.81 (s, 1H), 4.48 (q, J = 7.0 Hz, 2H), 4.06 (q, J = 7.2 Hz, 2H), 3.20-3.17 (m, 2H), 3.15-3.12 (m, 1H), 2.60-2.57 (m, 3H), 2.40-2.36 (m, 4H), 1.93-1.90 (m, 1H), 1.40 (t, J = 7.0 Hz, 3H), 1.16 (t, J = 7.1 Hz, 3H), 1.07-1.04 (m, 2H), 0.92 (d, J = 6.4 Hz, 2H). 1H obscured in DMSO. |
| P296 | 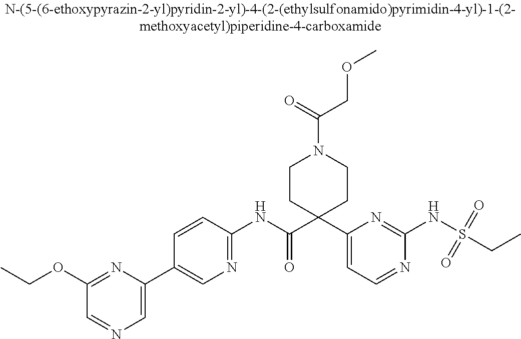 [SMILES: CCOc1cncc(-c2ccc(NC(=O)C3(c4ccnc(NS(=O)(=O)CC)n4)CCN(C(=O)COC)CC3)nc2)n1] | Prepared as for P288 using INTC235 and 2- methoxyacetyl chloride, [UPLC Acidic], 585, (1.18) | 11.24 (s, 1H), 10.23 (s, 1H), 9.04 (d, J = 2.5 Hz, 1H), 8.85 (s, 1H), 8.62 (s, 1H), 8.51 (dd, J = 8.7, 2.5 Hz, 1H), 8.26 (s, 1H), 8.21 (d, J = 8.8 Hz, 1H), 7.24 (s, 1H), 4.48 (q, J = 7.0 Hz, 2H), 4.20-4.02 (m, 2H), 3.89 (s, 1H), 3.59 (s, 1H), 3.51 (s, 2H), 3.41-3.33 (m, 1H), 3.29 (s, 3H), 3.22 (s, 1H), 2.24-1.95 (m, 2H), 1.40 (t, J = 7.0 Hz, 3H), 1.12 (t, J = 7.4 Hz, 3H), 0.89-0.80 (m, 2H). |
| P297 | 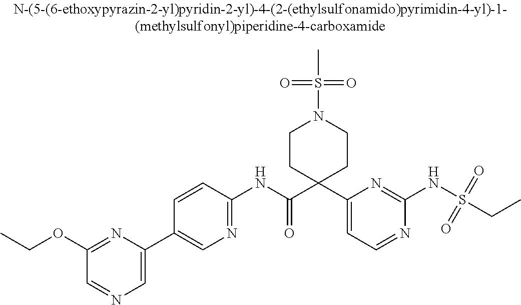 [SMILES: CCOc1cncc(-c2ccc(NC(=O)C3(c4ccnc(NS(=O)(=O)CC)n4)CCN(S(C)(=O)=O)CC3)nc2)n1] | Prepared as for P288 using INTC235 and methanesulfonyl chloride, [UPLC Acidic], 591, (1.26) | 11.25 (s, 1H), 10.27 (s, 1H), 9.03 (d, J = 2.5 Hz, 1H), 8.84 (s, 1H), 8.62 (d, J = 5.3 Hz, 1H), 8.51 (dd, J = 8.8, 2.5 Hz, 1H), 8.25 (s, 1H), 8.22 (d, J = 8.8 Hz, 1H), 7.24 (d, J = 5.3 Hz, 1H), 4.47 (q, J = 7.0 Hz, 2H), 3.53 (q, J = 7.3 Hz, 2H), 3.42 (d, J = 12.9 Hz, 2H), 3.09 (t, J = 10.7 Hz, 2H), 2.86 (s, 3H), 2.59-2.54 (m, 2H), 2.28-2.19 (m, 2H), 1.39 (t, J = 7.0 Hz, 3H), 1.13 (t, J = 7.3 Hz, 3H). |
| P298 | 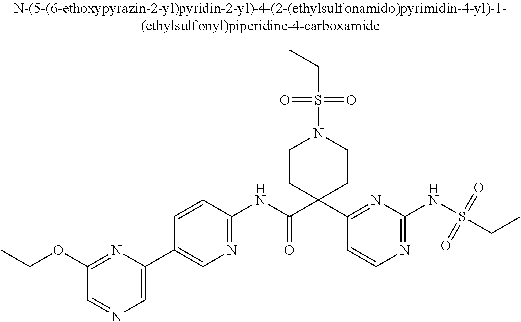 [SMILES: CCOc1cncc(-c2ccc(NC(=O)C3(c4ccnc(NS(=O)(=O)CC)n4)CCN(S(=O)(=O)CC)CC3)nc2)n1] | Prepared as for P288 using INTC235 and ethanesulfonyl chloride, [UPLC Acidic], 605, (1.34) | 11.24 (s, 1H), 10.25 (s, 1H), 9.03 (d, J = 2.5 Hz, 1H), 8.84 (s, 1H), 8.61 (d, J = 5.3 Hz, 1H), 8.50 (dd, J = 8.8, 2.5 Hz, 1H), 8.25 (s, 1H), 8.21 (d, J = 8.8 Hz, 1H), 7.23 (d, J = 5.3 Hz, 1H), 4.47 (q, J = 7.0 Hz, 2H), 3.56-3.43 (m, 4H), 3.20-3.12 (m, 2H), 3.04 (q, J = 7.4 Hz, 2H), 2.58-2.50 (m, 2H), 2.25-2.14 (m, 2H), 1.39 (t, J = 7.0 Hz, 3H), 1.18 (t, J = 7.4 Hz, 3H), 1.12 (t, J = 7.3 Hz, 3H). |
| P299 | 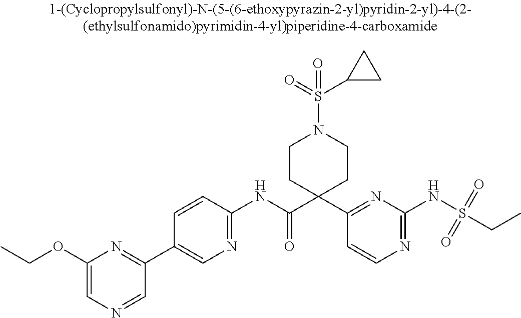 [SMILES: CCOc1cncc(-c2ccc(NC(=O)C3(c4ccnc(NS(=O)(=O)CC)n4)CCN(S(=O)(=O)C4CC4)CC3)nc2)n1] | Prepared as for P288 using INTC235 and cyclopropane sulfonyl chloride, [UPLC Acidic], 617, (1.35) | 11.27 (s, 1H), 10.28 (s, 1H), 9.04 (d, J = 2.4 Hz, 1H), 8.84 (s, 1H), 8.61 (d, J = 5.3 Hz, 1H), 8.51 (dd, J = 8.8, 2.5 Hz, 1H), 8.25 (s, 1H), 8.22 (d, J = 8.8 Hz, 1H), 7.23-7.20 (m, 1H), 4.48 (q, J = 7.0 Hz, 2H), 3.53-3.47 (m, 4H), 3.19 (t, J = 11.0 Hz, 2H), 2.67-2.51 (m, 4H), 2.27-2.18 (m, 1H), 1.40 (t, J = 7.0 Hz, 3H), 1.12 (t, J = 7.4 Hz, 3H), 0.99-0.88 (m, 4H) |
| P300 | 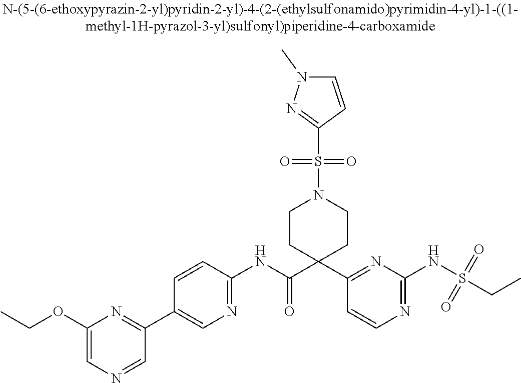 [SMILES: CCOc1cncc(-c2ccc(NC(=O)C3(c4ccnc(NS(=O)(=O)CC)n4)CCN(S(=O)(=O)c4ccn(C)n4)CC3)nc2)n1] | Prepared as for P288 using INTC235 and 1- methyl-1H- pyrazole-3- sulfony chloride, [UPLC Acidic], 657, (1.30) | 11.22 (s, 1H), 10.24 (s, 1H), 9.01 (dd, J = 2.4, 0.8 Hz, 1H), 8.83 (s, 1H), 8.60 (d, J = 5.3 Hz, 1H), 8.48 (dd, J = 8.7, 2.5 Hz, 1H), 8.25 (s, 1H), 8.10-8.05 (m, 1H), 7.90 (d, J = 2.3 Hz, 1H), 7.20 (d, J = 5.3 Hz, 1H), 6.65 (d, J = 2.3 Hz, 1H), 4.47 (q, J = 7.0 Hz, 2H), 3.88 (s, 3H), 3.53-3.39 (m, 4H), 2.93-2.84 (m, 2H), 2.60-2.53 (m, 2H), 2.24-2.15 (m, 2H), 1.39 (t, J = 7.0 Hz, 3H), 1.09 (t, J = 7.3 Hz, 3H). |
| P301 | 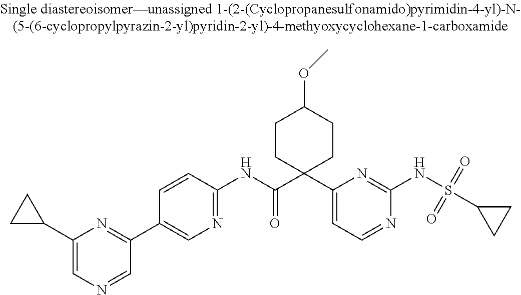 [SMILES: COC1CCC(C(=O)Nc2ccc(-c3cncc(C4CC4)n3)cn2)(c2ccnc(NS(=O)(=O)C3CC3)n2)CC1] | Method 11, using INTC218 and INTD54, [UPLC Acidic], 549, (1.37) | 11.28 (s, 1H), 10.01 (s, 1H), 9.01 (s, 1H), 8.98 (d, J = 2.4 Hz, 1H), 8.58 (s, 1H), 8.51-8.31 (m, 1H), 8.18 (d, J = 8.8 Hz, 1H), 7.17 (s, 1H), 3.24 (s, 3H), 2.78-2.53 (m, 2H), 2.33-2.15 (m, 1H), 2.03-1.77 (m, 4H), 1.55-1.33 (m, 2H), 1.22-0.98 (m, 6H), 0.91-0.75 (m, 2H). 1H not observed, 2H obscured under DMSO peak |
| P302 | 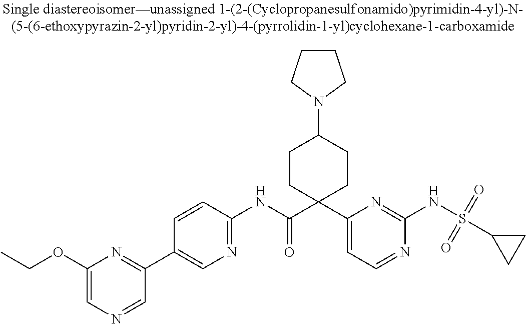 [SMILES: CCOc1cncc(-c2ccc(NC(=O)C3(c4ccnc(NS(=O)(=O)C5CC5)n4)CCC(N4CCCC4)CC3)nc2)n1] | Prepared as for P229 using P226 and pyrrolidine, [UPLC Acidic], 593, (0.91) | 9.56 (s, 1H), 9.01 (d, J = 2.4 Hz, 1H), 8.82 (s, 1H), 8.47 (dd, J = 8.8, 2.6 Hz, 2H), 8.24 (s, 1H), 8.18 (s, 2H), 8.09 (d, J = 8.8 Hz, 1H), 7.10 (s, 1H), 4.46 (q, J = 7.0 Hz, 2H), 3.15-3.11 (m, 1H), 2.87-2.83 (m, 3H), 2.71-2.68 (m, 2H), 2.02-1.99 (m, 3H), 1.96-1.92 (m, 2H), 1.78-1.75 (m, 5H), 1.45-1.41 (m, 2H), 1.38 (t, J = 7.0 Hz, 3H), 1.01-0.97 (m, 2H), 0.90-0.85 (m, 2H). Isolated as formate salt |
| P303 | 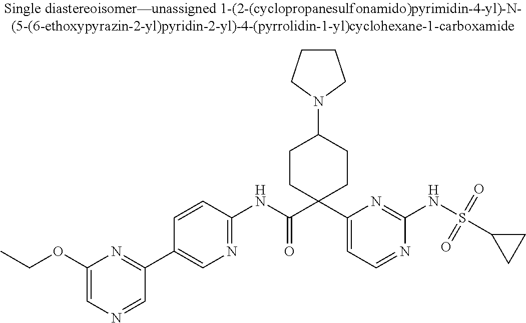 [SMILES: CCOc1cncc(-c2ccc(NC(=O)C3(c4ccnc(NS(=O)(=O)C5CC5)n4)CCC(N4CCCC4)CC3)nc2)n1] | Prepared as for P229 using P226 and pyrrolidine, [UPLC Acidic], 593, (0.85) | 10.19 (s, 1H), 9.04-9.00 (m, 1H), 8.84 (s, 1H), 8.52 (d, J = 5.3 Hz, 1H), 8.49 (dd, J = 8.8, 2.5 Hz, 1H), 8.26-8.23 (m, 3H), 8.23-8.17 (m, 1H), 7.06 (d, J = 5.3 Hz, 1H), 4.47 (q, J = 7.0 Hz, 2H), 3.28-3.19 (m, 1H), 2.73-2.57 (m, 5H), 2.05-1.99 (m, 3H), 1.85-1.73 (m, 7H), 1.65-1.48 (m, 2H), 1.39 (t, J = 7.0 Hz, 3H), 1.03-0.96 (m, 2H), 0.85-0.76 (m, 2H). Isolated as formate salt |
| P304 | 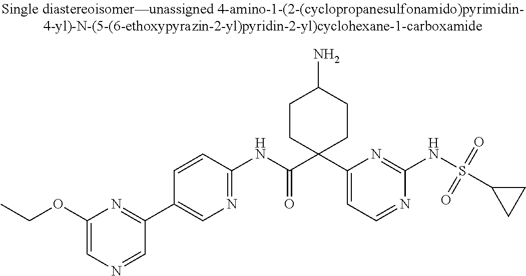 [SMILES: CCOc1cncc(-c2ccc(NC(=O)C3(c4ccnc(NS(=O)(=O)C5CC5)n4)CCC(N)CC3)nc2)n1] | Prepared as for P229 using P226 and ammonium acetate, [UPLC Acidic], 539, (0.78) | 10.22 (s, 1H), 9.05 (d, J = 2.5 Hz, 1H), 8.85 (s, 1H), 8.52 (dd, J = 8.8, 2.5 Hz, 1H), 8.33 (d, J = 5.2 Hz, 1H), 8.31 (s, 1H), 8.26 (s, 1H), 8.22 (d, J = 8.8 Hz, 1H), 6.70 (s, 1H), 4.49 (q, J = 7.1 Hz, 2H), 3.19-3.08 (m, 2H), 2.71-2.65 (m, 2H), 2.02-1.96 (m, 2H), 1.92-1.82 (m, 2H), 1.68-1.54 (m, 2H), 1.41 (t, J = 7.1 Hz, 3H), 0.91-0.86 (m, 2H), 0.71-0.65 (m, 2H), 2H not observed Isolated as partial formate salt |
| P305 | 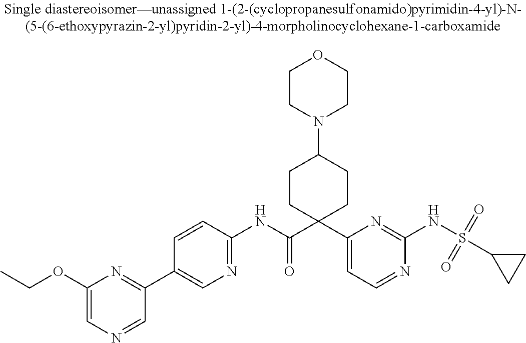 [SMILES: CCOc1cncc(-c2ccc(NC(=O)C3(c4ccnc(NS(=O)(=O)C5CC5)n4)CCC(N4CCOCC4)CC3)nc2)n1] | Prepared as for P229 using P226 and morpholine, [UPLC Basic], 609, (1.12) | 9.64 (s, 1H), 9.02 (d, J = 2.4 Hz, 1H), 8.83 (s, 1H), 8.62 (d, J = 5.3 Hz, 1H), 8.47 (dd, J = 8.8, 2.5 Hz, 1H), 8.25 (s, 1H), 8.16 (s, 1H formate), 8.10 (d, J = 8.8 Hz, 1H), 7.32 (d, J = 5.3 Hz, 1H), 4.47 (q, J = 7.0 Hz, 2H), 3.53 (t, J = 4.6 Hz, 4H), 3.30-3.21 (m, 1H), 2.71 (d, J = 13.3 Hz, 2H), 2.42-2.36 (m, 4H), 2.28-2.24 (m, 1H), 2.08-1.99 (m, 2H), 1.81-1.75 (m, 2H), 1.39 (t, J = 7.0 Hz, 3H), 1.34-1.24 (m, 2H), 1.10 (dt, J = 6.5, 3.3 Hz, 2H), 1.04-0.94 (m, 2H). 1H not observed. Isolated as formate salt. |
| P306 | 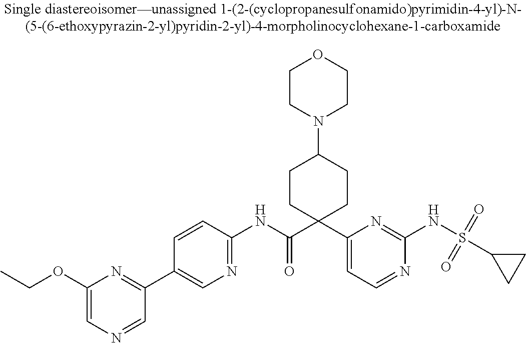 [SMILES: CCOc1cncc(-c2ccc(NC(=O)C3(c4ccnc(NS(=O)(=O)C5CC5)n4)CCC(N4CCOCC4)CC3)nc2)n1] | Prepared as for P229 using P226 and morpholine, [UPLC Basic], 609, (1.07) | 10.13, (s, 1H), 9.03-8.99 (m, 1H), 8.83 (s, 1H), 8.57 (d, J = 5.3 Hz, 1H), 8.49 (dd, J = 8.8, 2.5 Hz, 1H), 8.25 (s, 1H), 8.23-8.19 (m, 1H), 8.15 (s, 1H) formate, 7.16 (d, J = 5.3 Hz, 1H), 4.47 (q, J = 7.0 Hz, 2H), 3.57-3.51 (m, 4H), 3.30-3.21 (m, 1H), 2.72-2.66 (m, 2H), 2.45 (d, J = 5.0 Hz, 4H), 2.30-2.22 (m, 1H), 1.80-1.84 (m, 2H), 1.82-1.73 (m, 2H), 1.51-1.42 (m, 2H), 1.39 (t, J = 7.0 Hz, 3H), 1.04-0.97 (m, 2H), 0.86-0.78 (m, 2H). 1H not observed. Isolated as formate salt |
| P307 | 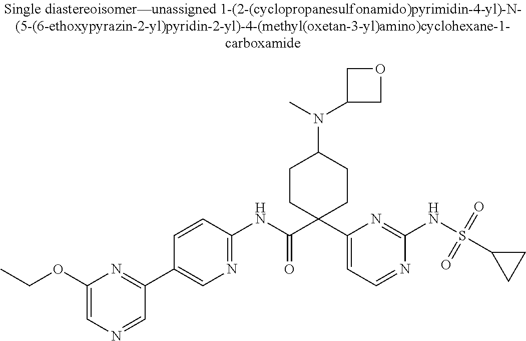 [SMILES: CCOc1cncc(-c2ccc(NC(=O)C3(c4ccnc(NS(=O)(=O)C5CC5)n4)CCC(N(C)C4COC4)CC3)nc2)n1] | Prepared as for P229 using P226 and N- methyloxetan-3- amine, [UPLC Basic], 609, (1.01) | 10.16 (s, 1H), 9.01 (d, J = 2.5 Hz, 1H), 8.83 (s, 1H), 8.55 (d, J = 5.3 Hz, 1H), 8.50 (dd, J = 8.8, 2.5 Hz, 1H), 8.25 (s, 1H), 8.21 (d, J = 8.8 Hz, 1H), 8.15 (s, 0.4H, partial formate salt), 7.13 (d, J = 5.4 Hz, 1H), 4.51-4.42 (m, 6H), 3.91-3.82 (m, 1H), 3.29-3.19 (m, 1H), 2.71-2.65 (m, 2H), 2.43-2.30 (m, 1H), 2.06 (s, 3H), 1.77-1.68 (m, 2H), 1.68-1.62 (m, 2H), 1.52-1.47 (m, 1H), 1.47-1.38 (m, 3H), 1.40-1.36 (m, 1H), 1.04-0.97 (m, 2H), 0.83-0.76 (m, 2H). 1H not observed. Isolated as partial formate salt |
| P308 | 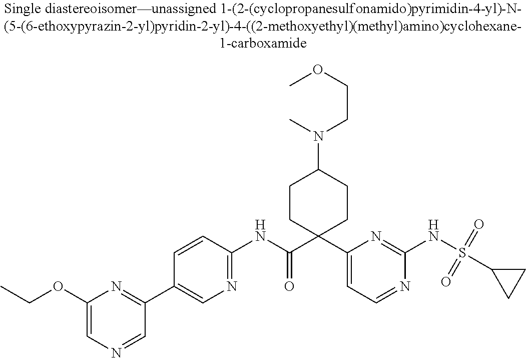 [SMILES: CCOc1cncc(-c2ccc(NC(=O)C3(c4ccnc(NS(=O)(=O)C5CC5)n4)CCC(N(C)CCOC)CC3)nc2)n1] | Prepared as for P229 using P226 and 2-methoxy-N- methylethan-1- amine, [UPLC Basic], 611, (0.98) | 10.14 (s, 1H), 9.06-9.00 (m, 1H), 8.84 (s, 1H), 8.55 (d, J = 5.4 Hz, 1H), 8.50 (dd, J = 8.7, 2.5 Hz, 1H), 8.30-8.24 (m, 1H), 8.22 (d, J = 8.8 Hz, 1H), 8.19 (s, 1H, formate), 7.13 (d, J = 5.4 Hz, 1H), 4.48 (q, J = 7.0 Hz, 2H), 3.37 (t, J = 6.1 Hz, 2H), 3.29-3.22 (m, 1H), 3.22 (s, 3H), 2.77-2.64 (m, 2H), 2.61-2.53 (m, 1H), 2.21 (s, 3H), 1.88-1.66 (m, 4H), 1.55-1.43 (m, 2H), 1.40 (t, J = 7.0 Hz, 3H), 1.05-0.95 (m, 2H), 0.85-0.77 (m, 2H). 1H not observed. Isolated as formate salt. 2H obscured by DMSO peak. |
| P309 | 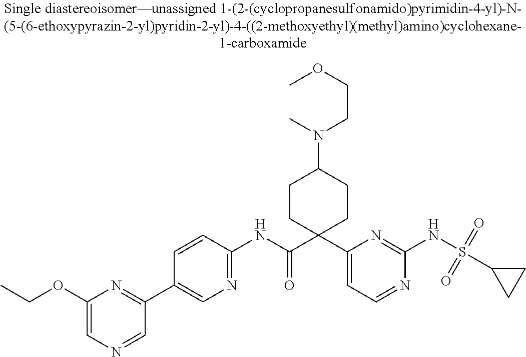 [SMILES: CCOc1cncc(-c2ccc(NC(=O)C3(c4ccnc(NS(=O)(=O)C5CC5)n4)CCC(N(C)CCOC)CC3)nc2)n1] | Prepared as for P229 using P226 and 2-methoxy-N- methylethan-1- amine, [UPLC Basic], 611, (1.02) | 9.60 (s, 1H), 9.02 (d, J = 2.5 Hz, 1H), 8.83 (s, 1H), 8.55 (s, 1H), 8.47 (dd, J = 9.0, 2.5 Hz, 1H), 8.25 (s, 1H), 8.18 (s, 1H formate), 8.08 (d, J = 8.8 Hz, 1H), 7.20-7.10 (m, 1H), 4.47 (q, J = 7.0 Hz, 2H), 3.26 (s, 1H), 3.21 (s, 3H), 2.78-2.72 (m, 2H), 2.13 (s, 3H), 2.02-1.92 (m, 2H), 1.73-1.67 (m, 2H), 1.39 (t, J = 7.0 Hz, 3H), 1.28-1.22 (m, 2H), 1.06-0.97 (m, 2H), 0.96-0.78 (m, 2H). 1H not observed. Isolated as formate salt. 5H obscured by DMSO and H₂O peaks |
| P310 | 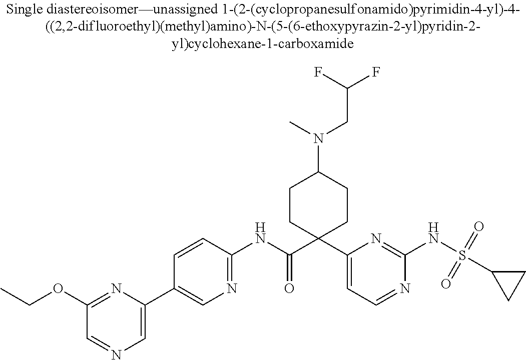 [SMILES: CCOc1cncc(-c2ccc(NC(=O)C3(c4ccnc(NS(=O)(=O)C5CC5)n4)CCC(N(C)CC(F)F)CC3)nc2)n1] | Prepared as for P229 using P226 and 2,2-difluoro- N-methylethan-1- amine, HCl, [UPLC Basic], 617, (1.33) | 10.16 (s, 1H), 9.02 (d, J = 2.5 Hz, 1H), 8.84 (s, 1H), 8.58-8.53 (m, 1H), 8.53-8.45 (m, 1H), 8.27-8.20 (m, 2H), 7.13 (d, J = 5.4 Hz, 1H), 6.14-5.86 (m, 1H), 4.48 (q, J = 7.0 Hz, 2H), 3.69-3.60 (m, 1H), 3.30-3.21 (m, 1H), 2.79-2.66 (m, 4H), 2.27 (s, 3H), 1.81-1.72 (m, 4H), 1.53-1.45 (m, 2H), 1.40 (t, J = 7.0 Hz, 3H), 1.05-0.99 (m, 2H), 0.85-0.77 (m, 2H). 1H not observed. Isolated as partial formate salt. |
| P311 | 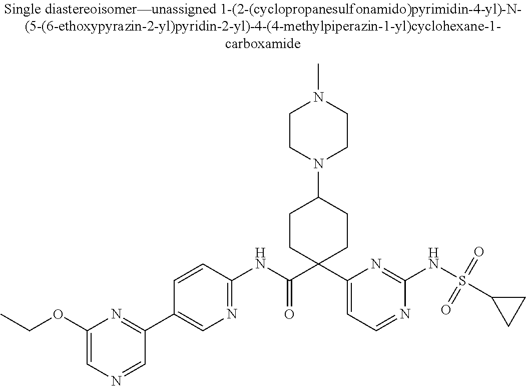 [SMILES: CCOc1cncc(-c2ccc(NC(=O)C3(c4ccnc(NS(=O)(=O)C5CC5)n4)CCC(N4CCN(C)CC4)CC3)nc2)n1] | Prepared as for P229 using P226 and 1- methylpiperazine, [UPLC Basic], 622, (0.97) | 9.60 (s, 1H), 9.02 (d, J = 2.5 Hz, 1H), 8.83 (s, 1H), 8.55 (s, 1H), 8.47 (dd, J = 9.0, 2.5 Hz, 1H), 8.25 (s, 1H), 8.18 (s, 1H formate), 8.08 (d, J = 8.8 Hz, 1H), 7.20-7.10 (m, 1H), 4.47 (q, J = 7.0 Hz, 2H), 3.26 (s, 1H), 3.21 (s, 3H), 2.78-2.72 (m, 2H), 2.13 (s, 3H), 2.02-1.92 (m, 2H), 1.73-1.67 (m, 2H), 1.39 (t, J = 7.0 Hz, 3H), 1.28-1.22 (m, 2H), 1.06-0.97 (m, 2H), 0.96-0.78 (m, 2H). 1H not observed. Isolated as formate salt. 5H obscured by DMSO and H₂O peaks |
| P312 | 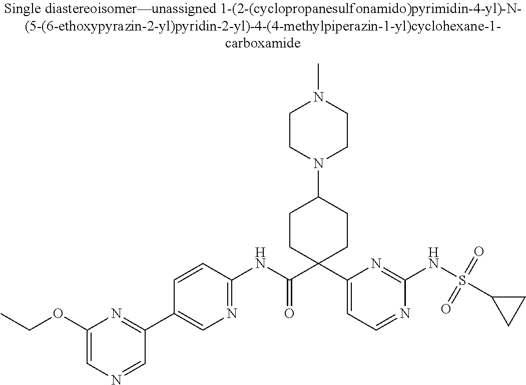 [SMILES: CCOc1cncc(-c2ccc(NC(=O)C3(c4ccnc(NS(=O)(=O)C5CC5)n4)CCC(N4CCN(C)CC4)CC3)nc2)n1] | Prepared as for P229 using P226 and 1- methylpiperazine, [UPLC Basic], 622, (0.91) | 10.12 (s, 1H), 9.04-9.00 (m, 1H), 8.84 (s, 1H), 8.57-8.53 (m, 1H), 8.53-8.47 (m, 1H), 8.28-8.23 (m, 1H), 8.23-8.16 (m, 1.5H) (formate), 7.13 (d, J = 5.4 Hz, 1H), 4.48 (q, J = 7.0 Hz, 2H), 3.28-3.21 (m, 1H), 2.72-2.66 (m, 2H), 2.42-2.25 (m, 8H), 2.16 (s, 3H), 1.84 (d, J = 12.5 Hz, 2H), 1.81-1.72 (m, 2H), 1.51-1.43 (m, 2H), 1.43-1.37 (m, 3H), 1.05-0.98 (m, 2H), 0.85-0.78 (m, 2H), 1H not observed. Isolated as partial formate salt. 1H obscured by DMSO |
| P313 | 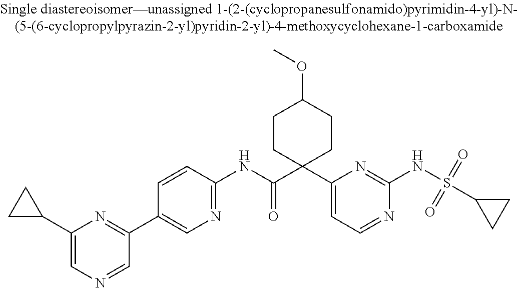 [SMILES: COC1CCC(C(=O)Nc2ccc(-c3cncc(C4CC4)n3)cn2)(c2ccnc(NS(=O)(=O)C3CC3)n2)CC1] | Method 11, using INTC218 and INTD54, [UPLC Acidic], 550, (1.42) | 11.29 (s, 1H), 9.88 (s, 1H), 9.00-8.98 (m, 2H), 8.60-8.58 (m, 2H), 8.45 (dd, J = 8.8, 2.5 Hz, 1H), 8.14 (d, J = 8.7 Hz, 1H), 7.20 (s, 1H) 3.22 (s, 3H), 2.38-2.34 (m, 2H), 2.32-2.18 (m, 3H), 1.82-1.75 (m, 2H), 1.58-1.50 (m, 2H), 1.29-1.24 (m, 2H), 1.10-1.05 (m, 5H), 0.92-0.85 (m, 3H) |
| P314 | 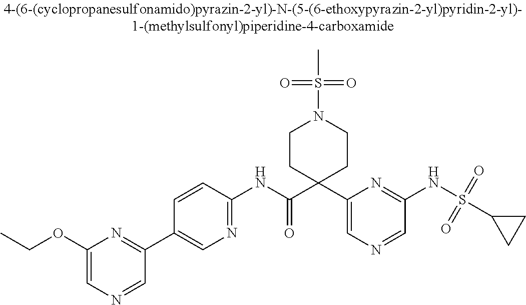 [SMILES: CCOc1cncc(-c2ccc(NC(=O)C3(c4cncc(NS(=O)(=O)C5CC5)n4)CCN(S(C)(=O)=O)CC3)nc2)n1] | Prepared as for P288 using INTC240 and methanesulfonyl chloride, [HPLC Acidic], 603, (2.02) | 11.08 (s, 1H), 10.26 (s, 1H), 9.01 (d, J = 2.4 Hz, 1H), 8.83 (s, 1H), 8.49 (dd, J = 8.8, 2.4 Hz, 1H), 8.42 (s, 1H), 8.25 (s, 1H), 8.23-8.17 (m, 2H), 4.47 (q, J = 7.0 Hz, 2H), 3.47-3.41 (m, 2H), 3.15-3.04 (m, 3H), 2.86 (s, 3H), 2.69-2.63 (m, 2H), 2.32-2.23 (m, 2H), 1.42-1.36 (m, 3H), 1.07-1.00 (m, 2H), 0.88-0.77 (m, 2H). |
| P315 | 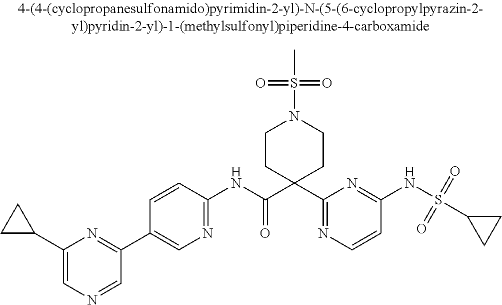 [SMILES: CS(=O)(=O)N1CCC(C(=O)Nc2ccc(-c3cncc(C4CC4)n3)cn2)(c2nccc(NS(=O)(=O)C3CC3)n2)CC1] | Prepared as for P288 using INTC245 and methanesulfonyl chloride, [HPLC Acidic], 599, (2.06) | 11.35 (s, 1H), 10.06 (s, 1H), 9.03-8.96 (m, 2H), 8.59 (d, J = 5.7 Hz, 2H), 8.47 (dd, J = 8.8, 2.4 Hz, 1H), 8.22 (d, J = 8.7 Hz, 1H), 6.87 (d, J = 5.7 Hz, 1H), 3.37-3.32 (m, 1H), 3.22-3.13 (m, 4H), 2.85 (s, 3H), 2.49-2.38 (m, 4H), 2.30-2.22 (m, 1H), 1.21-1.02 (m, 6H), 0.99-0.91 (m, 2H). |
| P316 | 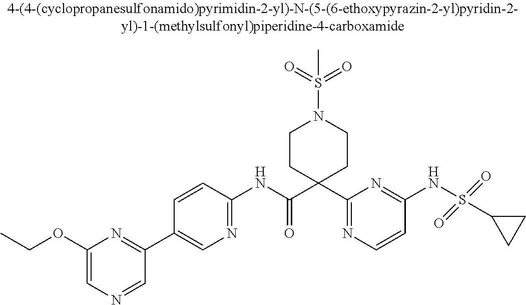 [SMILES: CCOc1cncc(-c2ccc(NC(=O)C3(c4nccc(NS(=O)(=O)C5CC5)n4)CCN(S(C)(=O)=O)CC3)nc2)n1] | Prepared as for P288 using INTC247 and methanesulfonyl chloride, [UPLC Acidic], 603, (1.33) | 11.35 (s, 1H), 10.08 (s, 1H), 9.02 (d, J = 2.4 Hz, 1H), 8.84 (s, 1H), 8.59 (d, J = 5.7 Hz, 1H), 8.53-8.47 (m, 1H), 8.27-8.20 (m, 2H), 6.88 (d, J = 5.7 Hz, 1H), 4.48 (q, J = 7.0 Hz, 2H), 3.34-3.30 (m, 2H), 3.22-3.14 (m, 3H), 2.85 (s, 3H), 2.50-2.38 (m, 4H), 1.40 (t, J = 7.0 Hz, 3H), 1.17-1.05 (m, 2H), 0.99-0.91 (m, 2H). |
| P317 | 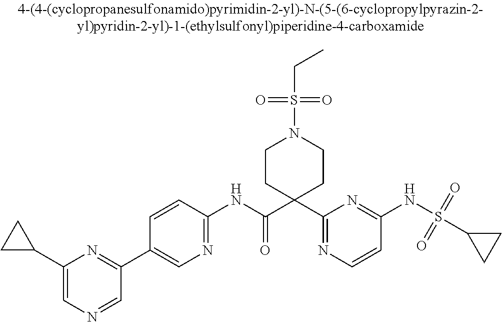 [SMILES: CCS(=O)(=O)N1CCC(C(=O)Nc2ccc(-c3cncc(C4CC4)n3)cn2)(c2nccc(NS(=O)(=O)C3CC3)n2)CC1] | Prepared as for P288 using INTC245 and ethanesulfonyl chloride, [HPLC Acidic], 613, (2.15) | 11.35 (s, 1H), 10.06 (s, 1H), 9.00 (s, 1H), 8.98 (d, J = 2.4 Hz, 1H), 8.61-8.56 (m, 2H), 8.47 (dd, J = 8.8, 2.4 Hz, 1H), 8.21 (d, J = 8.8 Hz, 1H), 6.87 (d, J = 5.7 Hz, 1H), 3.41-3.34 (m, 2H), 3.29-3.21 (m, 2H), 3.21-3.12 (m, 1H), 3.04 (q, J = 7.3 Hz, 2H), 2.48-2.34 (m, 4H), 2.30-2.22 (m, 1H), 1.19 (t, J = 7.3 Hz, 3H), 1.16-1.06 (m, 6H), 0.98-0.90 (m, 2H). |
| P318 | 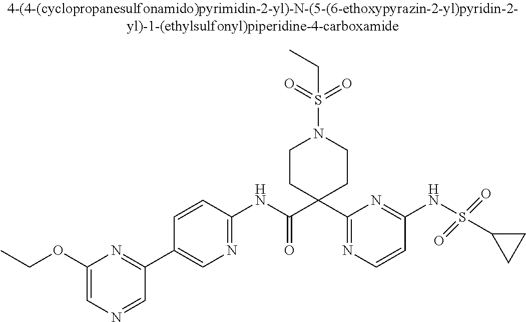 [SMILES: CCOc1cncc(-c2ccc(NC(=O)C3(c4nccc(NS(=O)(=O)C5CC5)n4)CCN(S(=O)(=O)CC)CC3)nc2)n1] | Prepared as for P288 using INTC247 and ethanesulfonyl chloride, [HPLC Acidic], 617, (2.16) | 11.35 (s, 1H), 10.07 (s, 1H), 9.02 (d, J = 2.4 Hz, 1H), 8.84 (s, 1H), 8.59-8.55 (m, 1H), 8.50 (dd, J = 8.7, 2.4 Hz, 1H), 8.25 (s, 1H), 8.22 (d, J = 8.8 Hz, 1H), 6.86 (d, J = 5.9 Hz, 1H), 4.48 (q, J = 7.0 Hz, 2H), 3.43-3.21 (m, 4H), 3.20-3.12 (m, 1H), 3.04 (q, J = 7.3 Hz, 2H), 2.47-2.37 (m, 4H), 1.40 (t, J = 7.0 Hz, 3H), 1.19 (t, J = 7.3 Hz, 3H), 1.09-1.05 (m, 2H), 0.96-0.91 (m, 2H). |

Compounds of Formula (I-b)—Intermediates

The synthesis of intermediates INTA1 to INTA117 and INTB1 to INTB120 is disclosed in WO2019/106156, which is herein incorporated in its entirety by reference, and uses the general methods shown below.

Method A: Formation of Thiazole Amines from Ketoesters

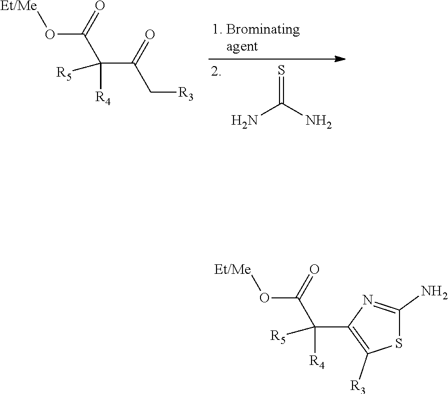

To a solution of ketoester (1 eq) in an alcoholic solvent such as MeOH or EtOH (1 volume) at 0° C. was added bromine (1.5 eq) dropwise over 10 mins. The reaction was stirred at 0° C. for 10 mins. The reaction mixture was then heated at 30° C. for 2 hrs. After cooling to RT the reaction mixture was diluted with water. The product was extracted using an appropriate solvent such as EtOAc. The combined organics were dried (Na₂SO₄) and concentrated in vacuo. The resulting compound was dissolved in alcoholic solvent such as MeOH or EtOH (1 volume) and thiourea (1 eq) was added. The reaction mixture was heated at 40° C. for 1 hr, then stirred at RT for 18 hrs. The reaction mixture was concentrated in vacuo and purified by normal phase chromatography or via trituration with an appropriate solvent.

Method B: Formation of Sulfonamides from Sulfonyl Chlorides

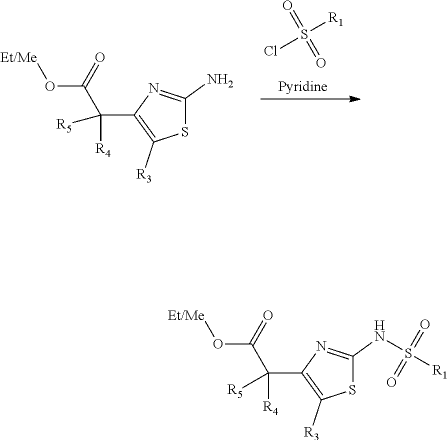

A solution of amine (1.0 eq) and appropriate sulfonyl chloride (1.1 eq) in pyridine (3M volumes) was warmed to 40° C. and stirred for 18 hrs. The reaction mixture was purified by normal or reverse phase chromatography or via trituration with an appropriate solvent.

Method C: Formation of Sulfonamides from Heterocyclic Halides

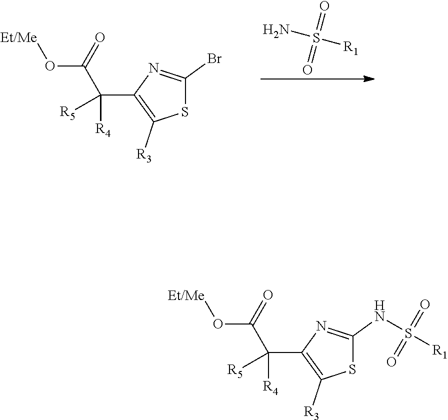

A suspension of (1R,2R)—N1,N2-dimethylcyclohexane-1,2-diamine (0.2 eq), bromothiazole intermediate (1 eq), alkylsulfonamide (1 eq), and K₂CO₃ (1.1 eq) in dioxane (10 volumes) at 40° C. was degassed (N₂, 5 mins) then copper(I) iodide (0.1 equiv.) was added. The solution was again degassed (N₂, 5 mins) before being warmed to 80° C. The reaction was progressed for 2 hrs before being allowed to cool to RT. 1M HCl (aq) was added and the aqueous phase was extracted with EtOAc. The organic phases were combined, dried (Na₂SO₄), filtered and concentrated. The crude material was purified by normal phase chromatography.

Method D: Formation of Acids from Esters

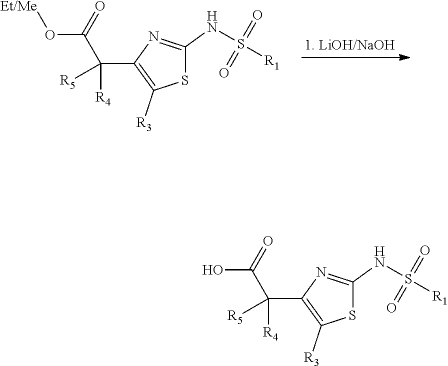

To a solution of ester (1.0 eq.) in THF/MeOH (2:1) was added a solution of appropriate base (2.0 eq. of LiOH or NaOH aq solutions at 1-2M). The reaction was stirred at RT or with heating up to 50° C. The reaction was concentrated in vacuo to half volume and was acidified with 1M HCl. The product was extracted using an appropriate organic solvent (EtOAc). The combined organics were dried (Na₂SO₄) and concentrated in vacuo to give the desired compound.

Aniline Intermediate Preparation

Method E: Suzuki Coupling of Halo Anilines with Heteroaromatic Boronates

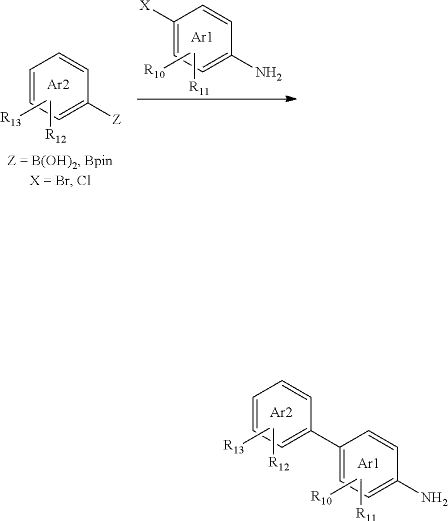

A solution of Ar1-X (1 eq) and Ar2-Z (1 eq) in solvent (3 volumes) and base (2.5 eq) was degassed (N₂, 5 min) and heated to 40° C. whereupon Pd catalyst (3 mol %) was added and the reaction mixture further degassed (N₂, 5 min) before being heated to 90° C. for 90 mins. The reaction mixture was allowed to cool to RT. In general, the desired compound is purified by column chromatography.

Method F: Suzuki Coupling of Heteroaromatic Halides with Aniline Boronates

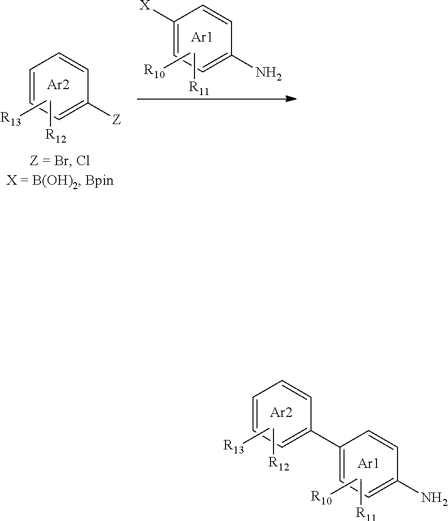

Pd catalyst (5 mol %) was added to a degassed (N₂, 5 mins) solution of Ar1-X (1 eq), Ar2-Z (1 eq) and base (3 eq, 6.85 mmol) in solvent (3 volumes). The solution was then degassed further (N₂, 5 mins) and then heated to 90° C. for 2 hrs then allowed to cool to RT. In general, the desired compound is purified by column chromatography.

Method G: Telescoped Boronate Formation and Suzuki Coupling

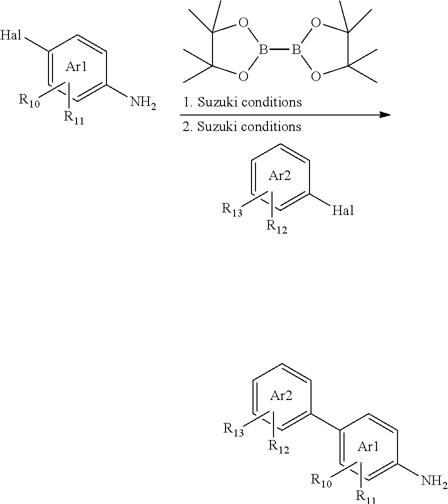

Bispin (1.1 eq) and KOAc (4 eq) were added to Ar1-Hal (1 eq) in dioxane (5 volumes). The reaction was heated to 60° C. and degassed (N₂, 5 mins). PdCl₂(dppf)-DCM adduct (5 mol %) was added to the reaction mixture and the temperature was increased to 90° C. for 1 hr. The reaction mixture was then cooled to RT and a solution of Ar2-Hal (1 eq) in dioxane (3 volumes) was added, followed by a solution of K₂CO₃ (4 eq) in water (2 volumes). The temperature was then increased to 90° C. for 18 hrs. The reaction was cooled to RT, an aqueous work up was performed and the crude compound was purified by normal phase chromatography.

Method H: Suzuki Coupling of Halo Pyrimidines with Heteroaromatic Boronates

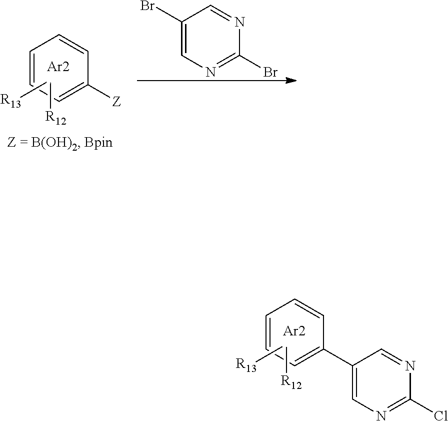

A solution of 5-bromo-2-chloropyrimidine (1.2 eq) and Ar2-Z (1 eq) in solvent (3 volumes) and base (4 eq) was degassed (N₂, 5 min) and heated to 40° C. whereupon Pd catalyst (5 mol %) was added and the reaction mixture further degassed (N₂, 5 min) before being heated to 90° C. for up to 24 hrs. The reaction mixture was allowed to cool to RT and an aqueous work-up was performed. In general, the desired compound is purified by column chromatography.

Compounds of Formula (I-b)—Examples

The synthesis of a number of known CTPS1 inhibitors is disclosed in WO2019/106156 (see compounds T1 to T465). Such compounds are made using general methods disclosed herein and represent further examples of compounds which are CTPS1 inhibitors. The full synthetic methods and characterising data for compounds T1 to T465 are provided in WO2019/106156.

Amide Couplings

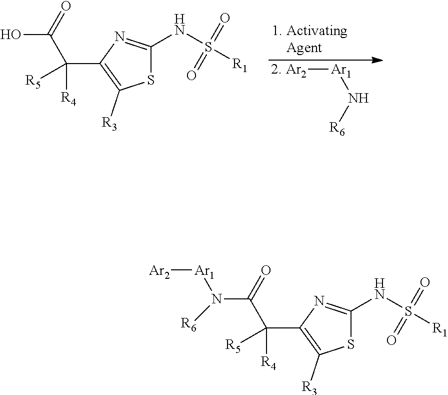

Method 1: HATU (1.2 eq) was added to a solution of appropriate acid (1 eq), amine (1 eq) and DIPEA (3 eq) in DCM (10 volumes) at RT. The reaction was stirred at RT for 18 hrs. The solvent was removed and the crude product was purified by normal phase chromatography, reverse phase chromatography or trituration from an appropriate solvent.

Method 1b: 1-chloro-N,N,2-trimethylprop-1-en-1-amine (2 eq) was added to a solution of 2-(2-(cydopropanesulfonamido)thiazol-4-yl)-2-methylpropanoic acid (1 eq) in DCM (20 volumes). The reaction mixture was stirred at RT for 2 hrs. The reaction mixture was concentrated in vacuo and the residue redissolved in pyridine (2 mL) before addition of the appropriate amine (1.1 eq). The reaction mixture was stirred at RT for 2 hrs. An aqueous work up was performed and the crude product was purified by normal phase chromatography, reverse phase chromatography or trituration from an appropriate solvent

Method 1c: T3P (50% in EtOAc, 2.5 eq) was added to a solution of appropriate acid (1 eq), amine (1 eq) and pyridine (3 eq) in a mixture of EtOAc (20 volumes) and DMF (10 volumes). The reaction was stirred for 1 hr at RT. An aqueous work up was performed and the crude product was purified by normal phase chromatography, reverse phase chromatography or trituration from an appropriate solvent.

Method 1e: Thionyl chloride (2 eq) was added to a solution of an appropriate acid (1 eq) in toluene (20 volumes) at 70° C. The reaction mixture was stirred at 70° C. for 1 hr. The reaction mixture was cooled to RT and concentrated to dryness. The resulting intermediate was redissolved in EtOAc (10 volumes) and a solution of amine (1.1 eq) in EtOAc (20 volumes) was added followed by triethylamine (2 eq). The reaction mixture was stirred at 40° C. for 16 hrs. An aqueous work up was performed and the crude product was purified by normal phase chromatography, reverse phase chromatography or trituration from an appropriate solvent

Suzuki Couplings

Method 2: Suzuki Coupling of Ar1-Bromide with Heteroaromatic Boronates

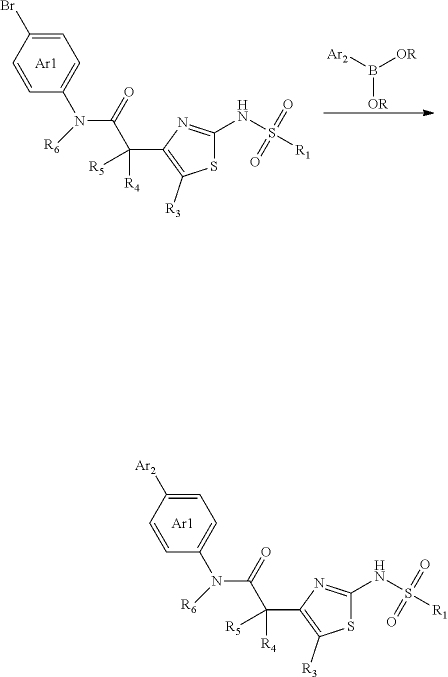

To a suspension of Ar1-Br (1 eq) in dioxane (10 volumes) was added arylboronic acid or ester (1 eq) and 2M K₂PO₄ (2 eq). The resulting suspension was heated to 60° C. and degassed (N₂, 5 mins). Pd 170 or other appropriate catalyst (5 mol %) was added and the reaction mixtures were stirred at 60° C. for 16 hrs. The reaction mixture was then cooled to RT. An aqueous work up was performed and the crude product was purified by normal phase chromatography, reverse phase chromatography or trituration from an appropriate solvent.

Method 2b: Suzuki Coupling of Ar1-B(OR)₂ with Heteroaromatic Halides

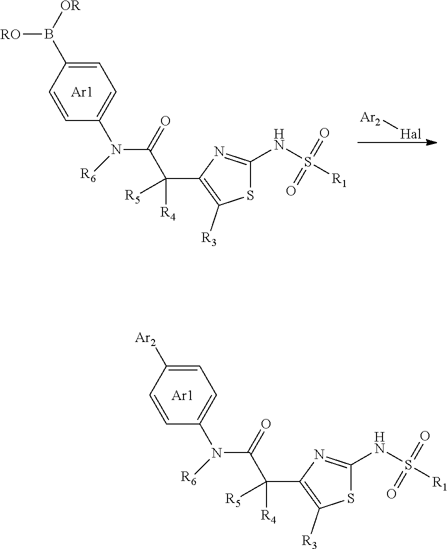

PdCl₂(dppf)-CH₂Cl₂ (10 mol %) or other appropriate catalyst was added to a degassed (N₂, 5 mins) solution of Ar1-B(OR)₂ (1 eq), Ar2-halide (1.2 eq) and K₂CO₃ (5 eq) in dioxane (10 volumes) and water (15 volumes). The solution was then degassed further (N₂, 5 mins) and then heated to 90° C. for 1-2 hrs. The reaction mixture was allowed to cool to RT. An aqueous work up was performed and the crude product was purified by normal phase chromatography, reverse phase chromatography or trituration from an appropriate solvent.

Method 3: Coupling of Primary Amides with 2-Chloropyrimidines

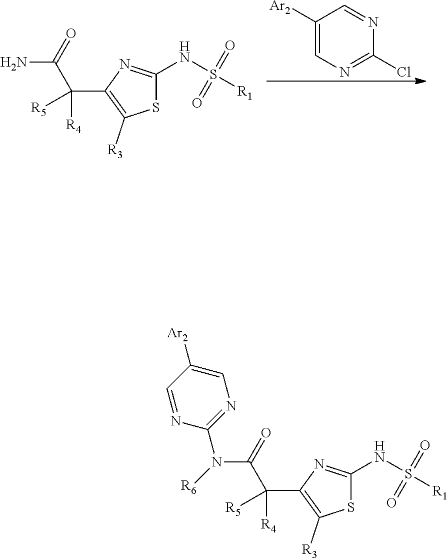

To a solution of amide (1 eq) and 2-chloropyrimidine (1 eq) in dioxane (30 volumes) was added Cs₂CO₃ (1.5 eq). The reaction mixture was heated to 60° C. and degassed (N₂, 5 mins). Pd 177 (10 mol %) was added to the reaction mixture and the temperature was increased to 90° C. After 2 hrs, the reaction was stirred for 16 hrs at 60° C. The reaction mixture was cooled to RT and an aqueous work up was performed. The crude product was purified by normal phase chromatography, reverse phase chromatography or trituration from an appropriate solvent.

### N-([1,1′-biphenyl]-4-yl)-2-(2-(methylsulfonamido)thiazol-4-yl)acetamide T1

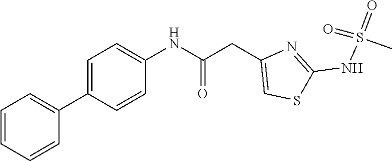 [SMILES: CS(=O)(=O)Nc1nc(CC(=O)Nc2ccc(-c3ccccc3)cc2)cs1]

### N-([1,1′-biphenyl]-4-yl)-2-(2-(cyclopropanesulfonamido)thiazol-4-yl)acetamide T2

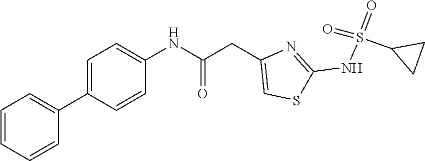 [SMILES: O=C(Cc1csc(NS(=O)(=O)C2CC2)n1)Nc1ccc(-c2ccccc2)cc1]

### 2-(2-(cyclopropanesulfonamido)thiazol-4-yl)-2-ethyl-N-(5-(pyrazin-2-yl)pyridin-2-yl)butanamide T3

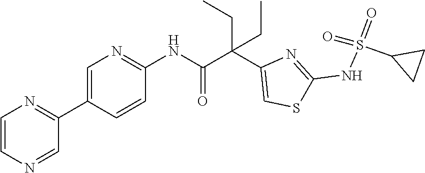 [SMILES: CCC(CC)(C(=O)Nc1ccc(-c2cnccn2)cn1)c1csc(NS(=O)(=O)C2CC2)n1]

Prepared as Method 1b from INTB41 and 5-(pyrazin-2-yl)pyridin-2-amine (Cheng et al., 2016).

### 2-(2-(cyclopropanesulfonamido)thiazol-4-yl)-2-methyl-N-(4-(pyrimidin-2-yl)phenyl)propanamide T4

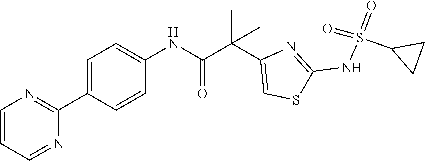 [SMILES: CC(C)(C(=O)Nc1ccc(-c2ncccn2)cc1)c1csc(NS(=O)(=O)C2CC2)n1]

### 2-(2-(cyclopropanesulfonamido)thiazol-4-yl)-N-(4-(pyridin-3-yl)phenyl)butanamide T5

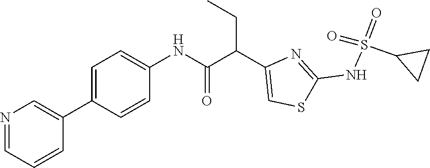 [SMILES: CCC(C(=O)Nc1ccc(-c2cccnc2)cc1)c1csc(NS(=O)(=O)C2CC2)n1]

The racemate T5 (180 mg) was separated by chiral preparative HPLC (Gilson, iso-hexane+0.2% TFA: DCM, 4:1 with EtOH 30%). A salt exchange (TFA to HCl) was undertaken by adding 1.25M HCl (EtOH, 2 mL×5) and removing solvent to afford:

Peak A: Stereochemistry of Product was not Assigned
2-(2-(cyclopropanesulfonamido)thiazol-4-yl)-N-(4-(pyridin-3-yl)phenyl)butanamide.HCl T6.

Peak B: Stereochemistry of Product was not Assigned
2-(2-(cydopropanesulfonamido)thiazol-4-yl)-N-(4-(pyridin-3-yl)phenyl)butanamide.HCl T7.
N-(4-(5-chloropyridin-3-yl)phenyl)-2-(2-(cydopropanesulfonamido)thiazol-4-yl)butanamide T8

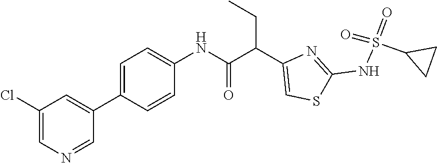 [SMILES: CCC(C(=O)Nc1ccc(-c2cncc(Cl)c2)cc1)c1csc(NS(=O)(=O)C2CC2)n1]

Prepared as Method 1 from INTB38.

The racemate T8 was separated by chiral preparative HPLC (30% EtOH vs 4:1 isoehexanes+0.2% TFA:DCM IA column). A salt exchange (TFA to HCl) was undertaken by adding 1.25M HCl (EtOH, 2 mL×5) and removing solvent to afford:

Peak A: Stereochemistry of Product was not Assigned
N-(4-(5-chloropyridin-3-yl)phenyl)-2-(2-(cyclopropanesulfonamido)thiazol-4-yl)butanamide.HCl.

Peak B: Stereochemistry of Product was not Assigned
N-(4-(5-chloropyridin-3-yl)phenyl)-2-(2-(cyclopropanesulfonamido)thiazol-4-yl)butanamide.HCl T10.
2-(2-(cyclopropanesulfonamido)thiazol-4-yl)-3-methyl-N-(4-(pyrimidin-5-yl)phenyl)butanamide T11

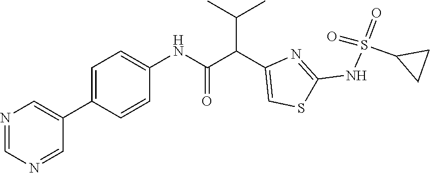 [SMILES: CC(C)C(C(=O)Nc1ccc(-c2cncnc2)cc1)c1csc(NS(=O)(=O)C2CC2)n1]

Prepared as Method 1 from INTB40.
2-(2-(cyclopropanesulfonamido)thiazol-4-yl)-3-methyl-N-(4-(pyridin-3-yl)phenyl)butanamide T12

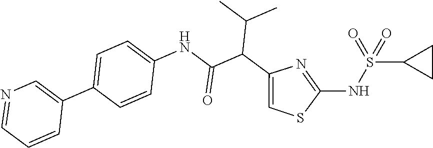 [SMILES: CC(C)C(C(=O)Nc1ccc(-c2cccnc2)cc1)c1csc(NS(=O)(=O)C2CC2)n1]

Prepared as Method 1 from INTB40 and 4-(pyridin-3-yl)aniline (Xing-Li et al, 2009)

The racemate T12 was separated by chiral preparative HPLC (Gilson, iso-hexane+0.2% TFA: DCM, 4:1 with EtOH 30%). A salt exchange (TFA to HCl) was undertaken by adding 1.25M HCl (EtOH, 2 mL×5) and removing solvent to afford:

Peak A: Stereochemistry of Product was not Assigned
2-(2-(cyclopropanesulfonamido)thiazol-4-yl)-3-methyl-N-(4-(pyridin-3-yl)phenyl)butanamide.HCl T13.

Peak B: Stereochemistry of Product was not Assigned
2-(2-(cyclopropanesulfonamido)thiazol-4-yl)-3-methyl-N-(4-(pyridin-3-yl)phenyl)butanamide.HCl T14.
2-(2-(cyclopropanesulfonamido)thiazol-4-yl)-N-(4-(5-methoxypyridin-3-yl)phenyl)-2-methylpropanamide T15

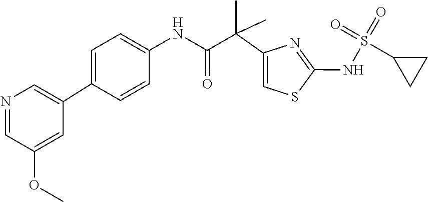 [SMILES: COc1cncc(-c2ccc(NC(=O)C(C)(C)c3csc(NS(=O)(=O)C4CC4)n3)cc2)c1]

N-(2-chloro-4-(pyridin-3-yl)phenyl)-2-(2-(cyclopropanesulfonamido)thiazol-4-yl)acetamide T16

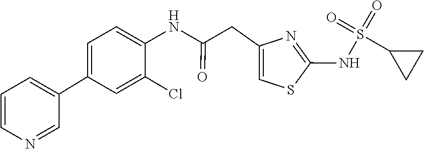 [SMILES: O=C(Cc1csc(NS(=O)(=O)C2CC2)n1)Nc1ccc(-c2cccnc2)cc1Cl]

2-(2-(cyclopropanesulfonamido)-5-methylthiazol-4-yl)-2-methyl-N-(4-(6-(trifluoromethyl)pyrazin-2-yl)phenyl)propanamide T17

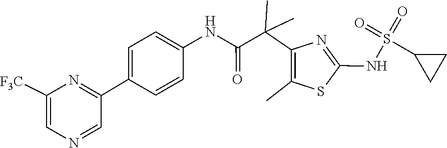 [SMILES: Cc1sc(NS(=O)(=O)C2CC2)nc1C(C)(C)C(=O)Nc1ccc(-c2cncc(C(F)(F)F)n2)cc1]

2-(2-(cyclopropanesulfonamido)thiazol-4-yl)-2-methyl-N-(4-(pyrimidin-5-yl)phenyl)propanamide T18

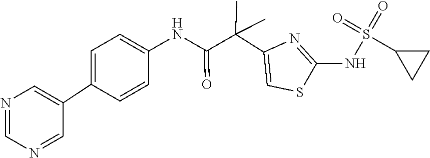 [SMILES: CC(C)(C(=O)Nc1ccc(-c2cncnc2)cc1)c1csc(NS(=O)(=O)C2CC2)n1]

6-(4-(2-(2-(cydopropanesulfonamido)thiazol-4-yl)-2-methylpropanamido)phenyl)-N,N-dimethylpyrazine-2-carboxamide T19

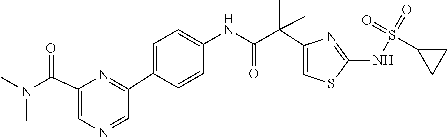 [SMILES: CN(C)C(=O)c1cncc(-c2ccc(NC(=O)C(C)(C)c3csc(NS(=O)(=O)C4CC4)n3)cc2)n1]

N-(5-(5-cyanopyridin-3-yl)pyrimidin-2-yl)-2-(2-(cyclopropanesulfonamido)thiazol-4-yl)-2-methylpropanamide T20

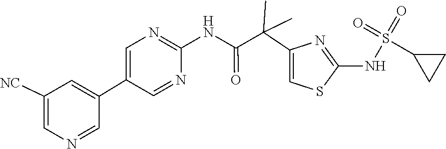 [SMILES: CC(C)(C(=O)Nc1ncc(-c2cncc(C#N)c2)cn1)c1csc(NS(=O)(=O)C2CC2)n1]

N-([1,1′-biphenyl]4-yl)-2-(5-chloro-2-(cydopropanesulfonamido)thiazol-4-yl)acetamide T21

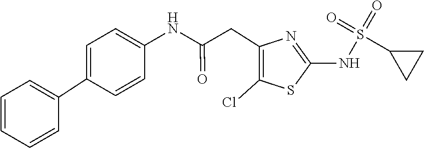 [SMILES: O=C(Cc1nc(NS(=O)(=O)C2CC2)sc1Cl)Nc1ccc(-c2ccccc2)cc1]

2-(2-(cyclopropanesulfonamido)thiazol-4-yl)-N-(4-(6-ethynylpyrazin-2-yl)phenyl)butanamide T22

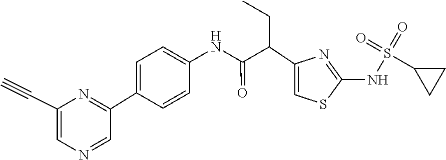 [SMILES: C#Cc1cncc(-c2ccc(NC(=O)C(CC)c3csc(NS(=O)(=O)C4CC4)n3)cc2)n1]

TABLE 6
Compounds T23-T322, T422-T443 and T445-T465.

| | Name/Structure |
|---|---|
| | (All examples containing chiral |
| | centres are racemates unless |
| T# | stated) |
| T23 | 2-(2- |
| | (cyclopropanesulfonamido)thiazol-4- |
| | yl)-N-(6-(pyrimidin-5-yl)pyridin-3- |
| | yl)acetamide |
| | 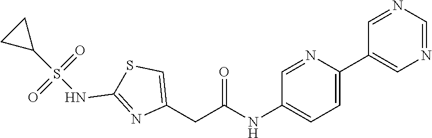 [SMILES: O=C(Cc1csc(NS(=O)(=O)C2CC2)n1)Nc1ccc(-c2cncnc2)nc1] |
| T24 | 2-(2- |
| | (cyclopropanesulfonamido)thiazol-4- |
| | yl)-N-(5-phenylpyridin-2-yl)acetamide |
| | 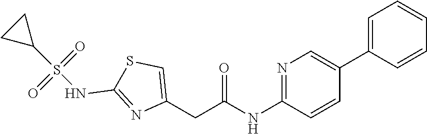 [SMILES: O=C(Cc1csc(NS(=O)(=O)C2CC2)n1)Nc1ccc(-c2ccccc2)cn1] |
| T25 | 2-(2- |
| | (cyclopropanesulfonamido)thiazol-4- |
| | yl)-N-(4′-fluoro-[1,1′-biphenyl]-4- |
| | yl)acetamide |
| | 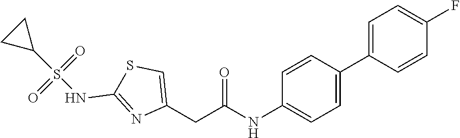 [SMILES: O=C(Cc1csc(NS(=O)(=O)C2CC2)n1)Nc1ccc(-c2ccc(F)cc2)cc1] |
| T26 | 2-(2- |
| | (cyclopropanesulfonamido)thiazol-4- |
| | yl)-N-methyl-N-(4-(pyridin-3- |
| | yl)phenyl)acetamide |
| | 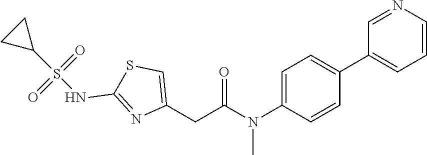 [SMILES: CN(C(=O)Cc1csc(NS(=O)(=O)C2CC2)n1)c1ccc(-c2cccnc2)cc1] |
| T27 | 2-([2,3′-bipyridin]-5-yl)-2-(2- |
| | (cyclopropanesulfonamido)thiazol-4- |
| | yl)propanamide |
| | 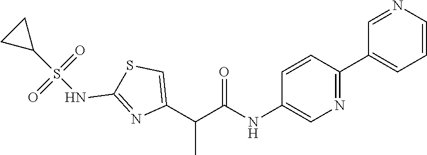 [SMILES: CC(C(=O)Nc1ccc(-c2cccnc2)nc1)c1csc(NS(=O)(=O)C2CC2)n1] |
| T28 | 2-(2- |
| | (cyclopropanesulfonamido)thiazol-4- |
| | yl)-N-(3′-methoxy-[1,1′-biphenyl]-4- |
| | yl)-2-methylpropanamide |
| | 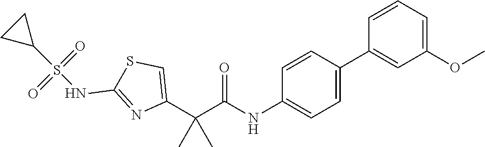 [SMILES: COc1cccc(-c2ccc(NC(=O)C(C)(C)c3csc(NS(=O)(=O)C4CC4)n3)cc2)c1] |
| T29 | 2-(2- |
| | (cyclopropanesulfonamido)thiazol-4- |
| | yl)-2-methyl-N-(4-(pyridin-3- |
| | yl)phenyl)propanamide |
| | 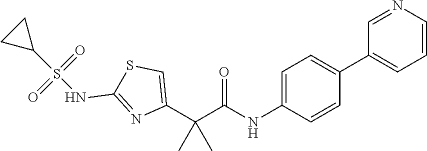 [SMILES: CC(C)(C(=O)Nc1ccc(-c2cccnc2)cc1)c1csc(NS(=O)(=O)C2CC2)n1] |
| T30 | 2-(2- |
| | (cyclopropanesulfonamido)thiazol-4- |
| | yl)-2-methyl-N-(4-(5-methylpyridin-3- |
| | yl)phenyl)propanamide |
| | 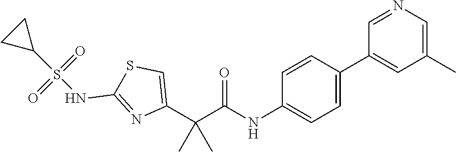 [SMILES: Cc1cncc(-c2ccc(NC(=O)C(C)(C)c3csc(NS(=O)(=O)C4CC4)n3)cc2)c1] |
| T31 | 2-(2- |
| | (cyclopropanesulfonamido)thiazol-4- |
| | yl)-2-methyl-N-(4-(pyridazin-4- |
| | yl)phenyl)propanamide |
| | 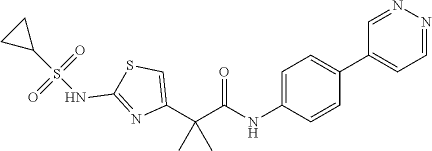 [SMILES: CC(C)(C(=O)Nc1ccc(-c2ccnnc2)cc1)c1csc(NS(=O)(=O)C2CC2)n1] |
| T32 | 2-(2- |
| | (cyclopropanesulfonamido)thiazol-4- |
| | yl)-2-methyl-N-(4-(pyrazin-2- |
| | yl)phenyl)propanamide |
| | 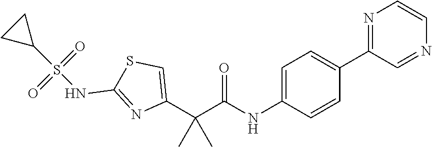 [SMILES: CC(C)(C(=O)Nc1ccc(-c2cnccn2)cc1)c1csc(NS(=O)(=O)C2CC2)n1] |
| T33 | 2-(2- |
| | (cyclopropanesulfonamido)thiazol-4- |
| | yl)-N-(4-(6-methoxypyrazin-2- |
| | yl)phenyl)butanamide |
| | 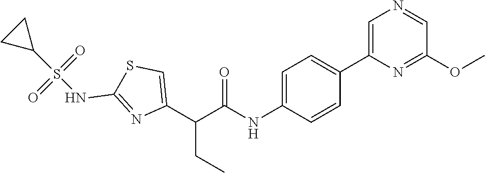 [SMILES: CCC(C(=O)Nc1ccc(-c2cncc(OC)n2)cc1)c1csc(NS(=O)(=O)C2CC2)n1] |
| T34 | N-(3-cyano-4-(pyrazin-2-yl)phenyl)-2- |
| | (2-(cyclopropanesulfonamido)thiazol- |
| | 4-yl)-2-methylpropanamide |
| | 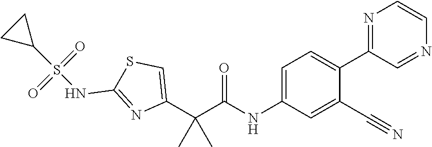 [SMILES: CC(C)(C(=O)Nc1ccc(-c2cnccn2)c(C#N)c1)c1csc(NS(=O)(=O)C2CC2)n1] |
| T35 | 2-(2- |
| | (cyclopropanesulfonamido)thiazol-4- |
| | yl)-2-methyl-N-(5-(6- |
| | (trifluoromethyl)pyrazin-2-yl)pyridin-2- |
| | yl)propanamide |
| | 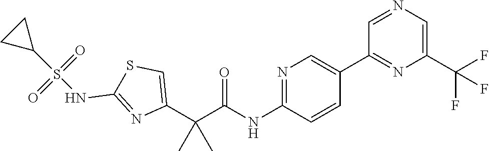 [SMILES: CC(C)(C(=O)Nc1ccc(-c2cncc(C(F)(F)F)n2)cn1)c1csc(NS(=O)(=O)C2CC2)n1] |
| T36 | 2-(2- |
| | (cyclopropanesulfonamido)thiazol-4- |
| | yl)-N-(2,3-difluoro-4-(pyridin-3- |
| | yl)phenyl)-2-methylpropanamide |
| | 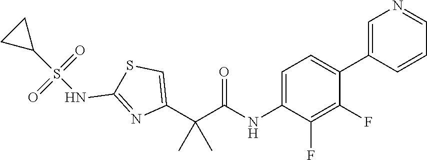 [SMILES: CC(C)(C(=O)Nc1ccc(-c2cccnc2)c(F)c1F)c1csc(NS(=O)(=O)C2CC2)n1] |
| T37 | 2-(2- |
| | (cyclopropanesulfonamido)thiazol-4- |
| | yl)-2-methyl-N-(5-(pyridin-3- |
| | yl)pyrimidin-2-yl)propanamide |
| | 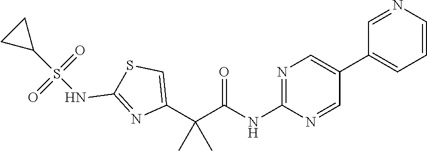 [SMILES: CC(C)(C(=O)Nc1ncc(-c2cccnc2)cn1)c1csc(NS(=O)(=O)C2CC2)n1] |
| T38 | 2-(2- |
| | (cyclopropanesulfonamido)thiazol-4- |
| | yl)-2-methyl-N-(5-(6-propoxypyrazin- |
| | 2-yl)pyridin-2-yl)propanamide |
| | 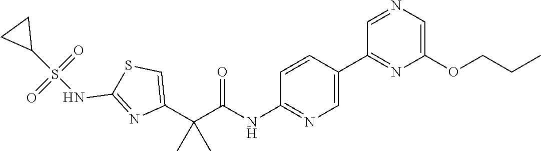 [SMILES: CCCOc1cncc(-c2ccc(NC(=O)C(C)(C)c3csc(NS(=O)(=O)C4CC4)n3)nc2)n1] |
| T39 | 2-(2- |
| | (cyclopropanesulfonamido)thiazol-4- |
| | yl)-N-(3-fluoro-4-(pyrazin-2- |
| | yl)phenyl)-2-methylpropanamide |
| | 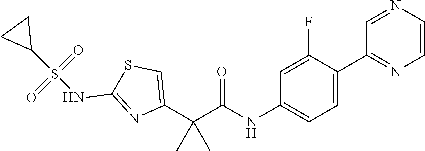 [SMILES: CC(C)(C(=O)Nc1ccc(-c2cnccn2)c(F)c1)c1csc(NS(=O)(=O)C2CC2)n1] |
| T40 | 2-(2- |
| | (cyclopropanesulfonamido)thiazol-4- |
| | yl)-2-methyl-N-(4-(pyridin-3-yl)-2- |
| | (trifluoromethoxy)phenyl)propanamide |
| | 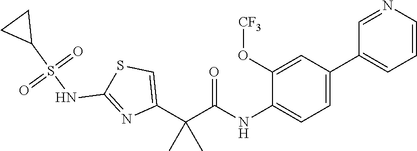 [SMILES: CC(C)(C(=O)Nc1ccc(-c2cccnc2)cc1OC(F)(F)F)c1csc(NS(=O)(=O)C2CC2)n1] |
| T41 | N-(2-chloro-4-(pyridin-3-yl)phenyl)-2- |
| | (2-(cyclopropanesulfonamido)thiazol- |
| | 4-yl)-2-methylpropanamide |
| | 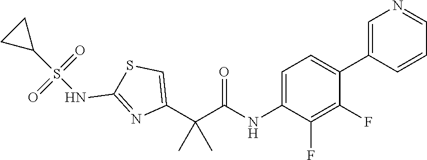 [SMILES: CC(C)(C(=O)Nc1ccc(-c2cccnc2)c(F)c1F)c1csc(NS(=O)(=O)C2CC2)n1] |
| T42 | 2-(2- |
| | (cyclopropanesulfonamido)thiazol-4- |
| | yl)-N-(2-fluoro-4-(pyridin-3-yl)phenyl)- |
| | 2-methylpropanamide |
| | 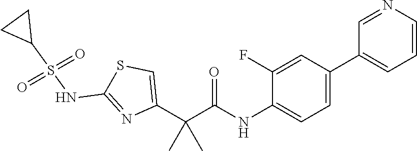 [SMILES: CC(C)(C(=O)Nc1ccc(-c2cccnc2)cc1F)c1csc(NS(=O)(=O)C2CC2)n1] |
| T43 | 2-(2- |
| | (cyclopropanesulfonamido)thiazol-4- |
| | yl)-N-(3-methoxy-4-(pyrazin-2- |
| | yl)phenyl)-2-methylpropanamide |
| | 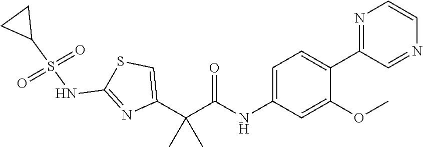 [SMILES: COc1cc(NC(=O)C(C)(C)c2csc(NS(=O)(=O)C3CC3)n2)ccc1-c1cnccn1] |
| T44 | 2-(2- |
| | (cyclopropanesulfonamido)thiazol-4- |
| | yl)-N-(4-(2-methoxypyridin-3- |
| | yl)phenyl)-2-methylpropanamide |
| | 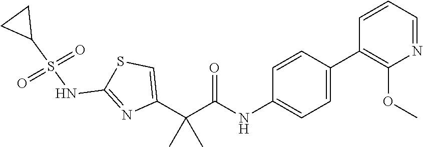 [SMILES: COc1ncccc1-c1ccc(NC(=O)C(C)(C)c2csc(NS(=O)(=O)C3CC3)n2)cc1] |
| T45 | 2-(2- |
| | (cyclopropanesulfonamido)thiazol-4- |
| | yl)-N-(4-(5-(hydroxymethyl)pyridin-3- |
| | yl)phenyl)acetamide |
| | 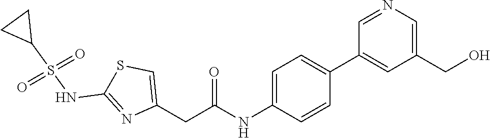 [SMILES: O=C(Cc1csc(NS(=O)(=O)C2CC2)n1)Nc1ccc(-c2cncc(CO)c2)cc1] |
| T46 | 2-(2- |
| | (cyclopropanesulfonamido)thiazol-4- |
| | yl)-N-(4-(6-methoxypyridin-3- |
| | yl)phenyl)-2-methylpropanamide |
| | 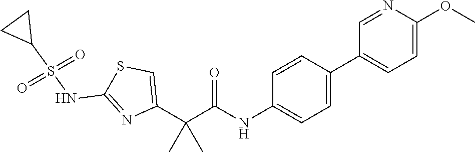 [SMILES: COc1ccc(-c2ccc(NC(=O)C(C)(C)c3csc(NS(=O)(=O)C4CC4)n3)cc2)cn1] |
| T47 | N-(4-(5-cyanopyridin-3-yl)phenyl)-2- |
| | (2-(cyclopropanesulfonamido)thiazol- |
| | 4-yl)-2-methylpropanamide |
| | 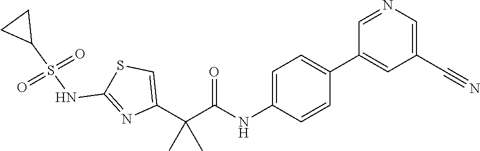 [SMILES: CC(C)(C(=O)Nc1ccc(-c2cncc(C#N)c2)cc1)c1csc(NS(=O)(=O)C2CC2)n1] |
| T48 | 2-(2- |
| | (cyclopropanesulfonamido)thiazol-4- |
| | yl)-2-methyl-N-(4-(5- |
| | (trifluoromethyl)pyridin-3- |
| | yl)phenyl)propanamide |
| | 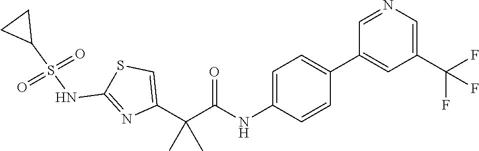 [SMILES: CC(C)(C(=O)Nc1ccc(-c2cncc(C(F)(F)F)c2)cc1)c1csc(NS(=O)(=O)C2CC2)n1] |
| T49 | 2-(2- |
| | (cyclopropanesulfonamido)thiazol-4- |
| | yl)-2-methyl-N-(4-(5- |
| | (methylsulfonyl)pyridin-3- |
| | yl)phenyl)propanamide |
| | 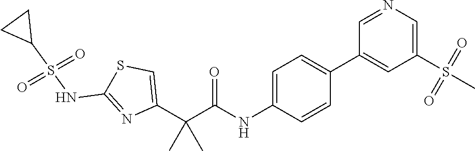 [SMILES: CC(C)(C(=O)Nc1ccc(-c2cncc(S(C)(=O)=O)c2)cc1)c1csc(NS(=O)(=O)C2CC2)n1] |
| T50 | 2-(2- |
| | (cyclopropanesulfonamido)thiazol-4- |
| | yl)-N-(2-methoxy-4-(5- |
| | methoxypyridin-3-yl)phenyl)-2- |
| | methylpropanamide |
| | 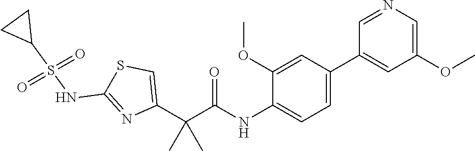 [SMILES: COc1cncc(-c2ccc(NC(=O)C(C)(C)c3csc(NS(=O)(=O)C4CC4)n3)c(OC)c2)c1] |
| T51 | 2-(2- |
| | (cyclopropanesulfonamido)thiazol-4- |
| | yl)-2-methyl-N-(6-(trifluoromethyl)- |
| | [2,3′-bipyridin]-6′-yl)propanamide |
| | 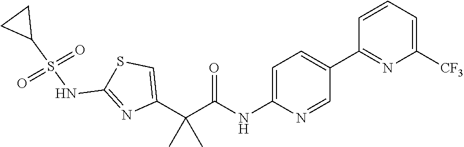 [SMILES: CC(C)(C(=O)Nc1ccc(-c2cccc(C(F)(F)F)n2)cn1)c1csc(NS(=O)(=O)C2CC2)n1] |
| T52 | 2-(2- |
| | (cyclopropanesulfonamido)thiazol-4- |
| | yl)-N-(4-(6-ethoxypyrazin-2- |
| | yl)phenyl)-2-methylpropanamide |
| | 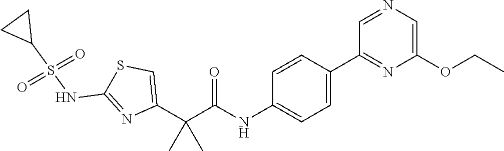 [SMILES: CCOc1cncc(-c2ccc(NC(=O)C(C)(C)c3csc(NS(=O)(=O)C4CC4)n3)cc2)n1] |
| T53 | 2-(2- |
| | (cyclopropanesulfonamido)thiazol-4- |
| | yl)-2-methyl-N-(4-(6- |
| | morpholinopyrazin-2- |
| | yl)phenyl)propanamide |
| | 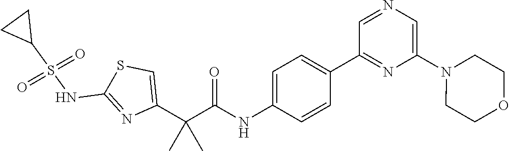 [SMILES: CC(C)(C(=O)Nc1ccc(-c2cncc(N3CCOCC3)n2)cc1)c1csc(NS(=O)(=O)C2CC2)n1] |
| T54 | N-(4-(6-cyclobutoxypyrazin-2- |
| | yl)phenyl)-2-(2- |
| | (cyclopropanesulfonamido)thiazol-4- |
| | yl)-2-methylpropanamide |
| | 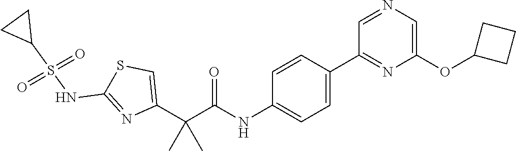 [SMILES: CC(C)(C(=O)Nc1ccc(-c2cncc(OC3CCC3)n2)cc1)c1csc(NS(=O)(=O)C2CC2)n1] |
| T55 | 2-(2- |
| | (cyclopropanesulfonamido)thiazol-4- |
| | yl)-2-methyl-N-(4-(6-propoxypyrazin- |
| | 2-yl)phenyl)propanamide |
| | 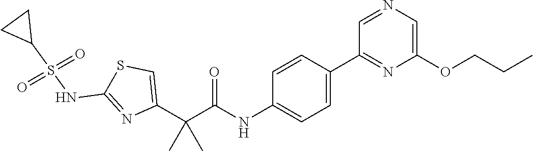 [SMILES: CCCOc1cncc(-c2ccc(NC(=O)C(C)(C)c3csc(NS(=O)(=O)C4CC4)n3)cc2)n1] |
| T56 | 2-(2- |
| | (cyclopropanesulfonamido)thiazol-4- |
| | yl)-N-(2-fluoro-4-(5-methoxypyridin-3- |
| | yl)phenyl)-2-methylpropanamide |
| | 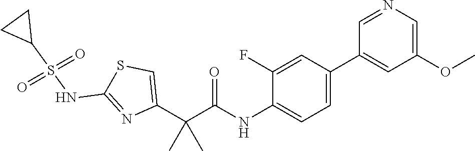 [SMILES: COc1cncc(-c2ccc(NC(=O)C(C)(C)c3csc(NS(=O)(=O)C4CC4)n3)c(F)c2)c1] |
| T57 | 2-(2- |
| | (cyclopropanesulfonamido)thiazol-4- |
| | yl)-2-methoxy-N-(4-(6- |
| | methoxypyrazin-2- |
| | yl)phenyl)acetamide |
| | 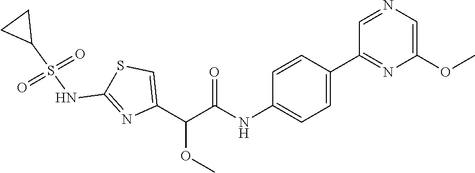 [SMILES: COc1cncc(-c2ccc(NC(=O)C(OC)c3csc(NS(=O)(=O)C4CC4)n3)cc2)n1] |
| T58 | N-(4-(5-chloropyridin-3-yl)phenyl)-2- |
| | (2-(cyclopropanesulfonamido)thiazol- |
| | 4-yl)-2-isopropoxyacetamide |
| | 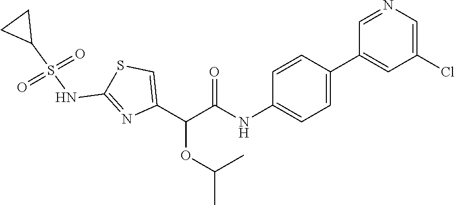 [SMILES: CC(C)OC(C(=O)Nc1ccc(-c2cncc(Cl)c2)cc1)c1csc(NS(=O)(=O)C2CC2)n1] |
| T59 | 2-(2- |
| | (cyclopropanesulfonamido)thiazol-4- |
| | yl)-4-methoxy-N-(5-(6- |
| | (trifluoromethyl)pyrazin-2-yl)pyridin-2- |
| | yl)butanamide |
| | 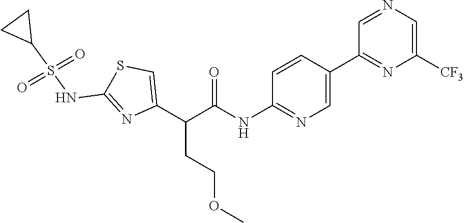 [SMILES: COCCC(C(=O)Nc1ccc(-c2cncc(C(F)(F)F)n2)cn1)c1csc(NS(=O)(=O)C2CC2)n1] |
| T60 | N-([1,1′-biphenyl]-4-yl)-2-(2- |
| | (cyclopropanesulfonamido)thiazol-4- |
| | yl)-2,2-difluoroacetamide |
| | 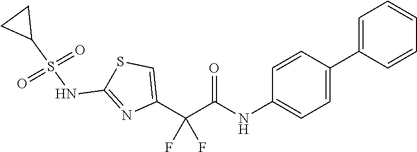 [SMILES: O=C(Nc1ccc(-c2ccccc2)cc1)C(F)(F)c1csc(NS(=O)(=O)C2CC2)n1] |
| T61 | 2-(2-(cyclobutanesulfonamido)thiazol- |
| | 4-yl)-N-(4-(6-methoxypyrazin-2- |
| | yl)phenyl)acetamide |
| | 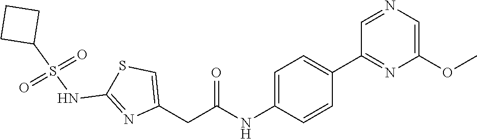 [SMILES: COc1cncc(-c2ccc(NC(=O)Cc3csc(NS(=O)(=O)C4CCC4)n3)cc2)n1] |
| T62 | N-([3,3′-bipyridin]-6-yl)-2-(2- |
| | (cyclopropanesulfonamido)thiazol-4- |
| | yl)butanamide |
| | 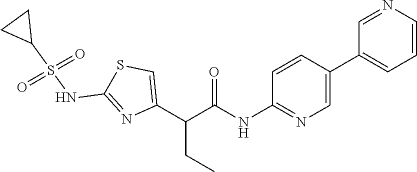 [SMILES: CCC(C(=O)Nc1ccc(-c2cccnc2)cn1)c1csc(NS(=O)(=O)C2CC2)n1] |
| T63 | 2-(2- |
| | (cyclopropanesulfonamido)thiazol-4- |
| | yl)-2-methyl-N-(5-phenylpyridin-2- |
| | yl)propanamide |
| | 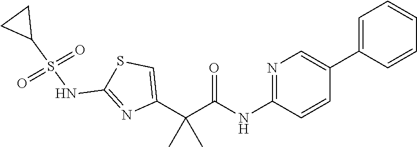 [SMILES: CC(C)(C(=O)Nc1ccc(-c2ccccc2)cn1)c1csc(NS(=O)(=O)C2CC2)n1] |
| T64 | 2-(2- |
| | (cyclopropanesulfonamido)thiazol-4- |
| | yl)-N-(5-(pyrimidin-5-yl)pyridin-2- |
| | yl)acetamide |
| | 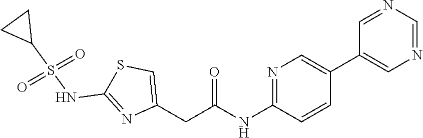 [SMILES: O=C(Cc1csc(NS(=O)(=O)C2CC2)n1)Nc1ccc(-c2cncnc2)cn1] |
| T65 | N-([3,3′-bipyridin]-6-yl)-2-(2- |
| | (cyclopropanesulfonamido)thiazol-4- |
| | yl)acetamide |
| | 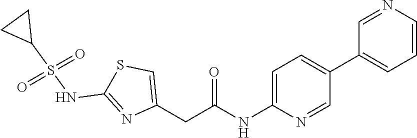 [SMILES: O=C(Cc1csc(NS(=O)(=O)C2CC2)n1)Nc1ccc(-c2cccnc2)cn1] |
| T66 | 2-(2- |
| | (cyclopropanesulfonamido)thiazol-4- |
| | yl)-N-(6-phenylpyridin-3-yl)acetamide |
| | 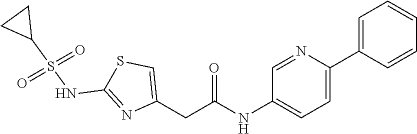 [SMILES: O=C(Cc1csc(NS(=O)(=O)C2CC2)n1)Nc1ccc(-c2ccccc2)nc1] |
| T67 | N-([2,3′-bipyridin]-5-yl)-2-(2- |
| | (cyclopropanesulfonamido)thiazol-4- |
| | yl)acetamide |
| | 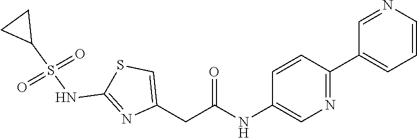 [SMILES: O=C(Cc1csc(NS(=O)(=O)C2CC2)n1)Nc1ccc(-c2cccnc2)nc1] |
| T68 | 2-(2- |
| | (cyclopropanesulfonamido)thiazol-4- |
| | yl)-2-methyl-N-(4-(pyridazin-3- |
| | yl)phenyl)propanamide |
| | 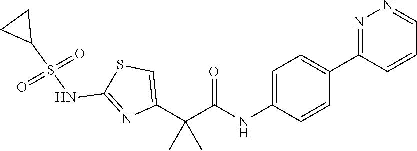 [SMILES: CC(C)(C(=O)Nc1ccc(-c2cccnn2)cc1)c1csc(NS(=O)(=O)C2CC2)n1] |
| T69 | 2-(2- |
| | (cyclopropanesulfonamido)thiazol-4- |
| | yl)-N-(4-(pyridazin-4- |
| | yl)phenyl)butanamide |
| | 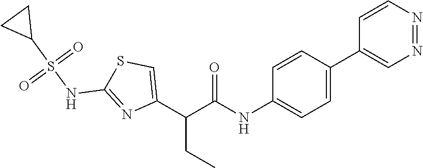 [SMILES: CCC(C(=O)Nc1ccc(-c2ccnnc2)cc1)c1csc(NS(=O)(=O)C2CC2)n1] |
| T70 | 2-(2- |
| | (cyclopropanesulfonamido)thiazol-4- |
| | yl)-N-(4-(pyrazin-2- |
| | yl)phenyl)butanamide |
| | 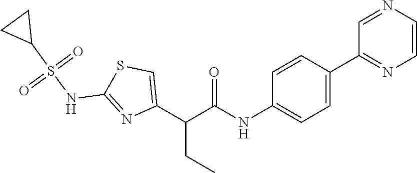 [SMILES: CCC(C(=O)Nc1ccc(-c2cnccn2)cc1)c1csc(NS(=O)(=O)C2CC2)n1] |
| T71 | N-(4-(5-chloropyridin-3-yl)phenyl)-2- |
| | (2-(cyclopropanesulfonamido)thiazol- |
| | 4-yl)-4-methoxybutanamide |
| | 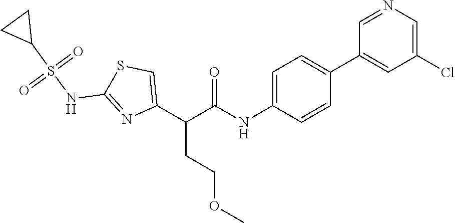 [SMILES: COCCC(C(=O)Nc1ccc(-c2cncc(Cl)c2)cc1)c1csc(NS(=O)(=O)C2CC2)n1] |
| T72 | Single enantiomer— |
| | stereochemistry unassigned 2-(2- |
| | (cyclopropanesulfonamido)thiazol-4- |
| | yl)-N-(4-(5-fluoropyridin-3- |
| | yl)phenyl)butanamide |
| | 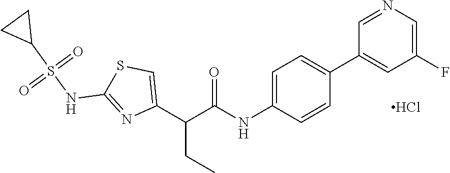 [SMILES: CCC(C(=O)Nc1ccc(-c2cncc(F)c2)cc1)c1csc(NS(=O)(=O)C2CC2)n1 ; Cl] |
| T73 | 2-(2- |
| | (cyclopropanesulfonamido)thiazol-4- |
| | yl)-N-(4-(6-(trifluoromethyl)pyrazin-2- |
| | yl)phenyl)butanamide |
| | 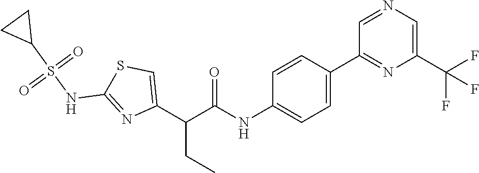 [SMILES: CCC(C(=O)Nc1ccc(-c2cncc(C(F)(F)F)n2)cc1)c1csc(NS(=O)(=O)C2CC2)n1] |
| T74 | 2-(2- |
| | (cyclopropanesulfonamido)thiazol-4- |
| | yl)-3-methyl-N-(4-(pyrazin-2- |
| | yl)phenyl)butanamide |
| | 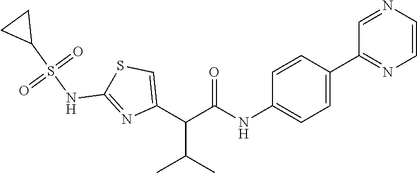 [SMILES: CC(C)C(C(=O)Nc1ccc(-c2cnccn2)cc1)c1csc(NS(=O)(=O)C2CC2)n1] |
| T75 | 2-(2- |
| | (cyclopropanesulfonamido)thiazol-4- |
| | yl)-N-(4-(6-propoxypyrazin-2- |
| | yl)phenyl)butanamide |
| | 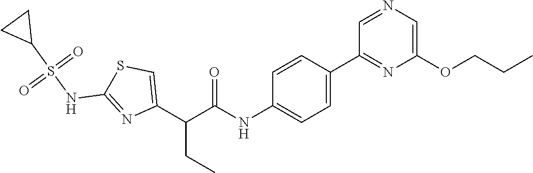 [SMILES: CCCOc1cncc(-c2ccc(NC(=O)C(CC)c3csc(NS(=O)(=O)C4CC4)n3)cc2)n1] |
| T76 | 2-(2- |
| | (cyclopropanesulfonamido)thiazol-4- |
| | yl)-N-(4-(6-isopropoxypyrazin-2- |
| | yl)phenyl)butanamide |
| | 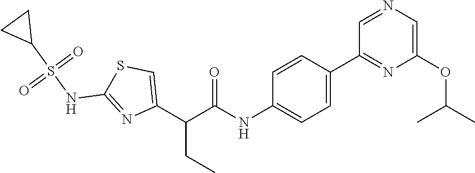 [SMILES: CCC(C(=O)Nc1ccc(-c2cncc(OC(C)C)n2)cc1)c1csc(NS(=O)(=O)C2CC2)n1] |
| T77 | 2-(2- |
| | (cyclopropanesulfonamido)thiazol-4- |
| | yl)-N-(4-(6-cyclopropoxypyrazin-2- |
| | yl)phenyl)butanamide |
| | 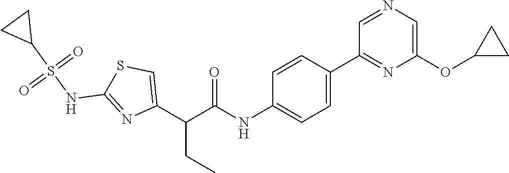 [SMILES: CCC(C(=O)Nc1ccc(-c2cncc(OC3CC3)n2)cc1)c1csc(NS(=O)(=O)C2CC2)n1] |
| T78 | N-(4-(6-chloropyrazin-2-yl)phenyl)-2- |
| | (2-(cyclopropanesulfonamido)thiazol- |
| | 4-yl)butanamide |
| | 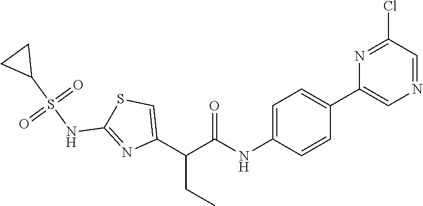 [SMILES: CCC(C(=O)Nc1ccc(-c2cncc(Cl)n2)cc1)c1csc(NS(=O)(=O)C2CC2)n1] |
| T79 | N-(4-(6-cyanopyrazin-2-yl)phenyl)-2- |
| | (2-(cyclopropanesulfonamido)thiazol- |
| | 4-yl)butanamide |
| | 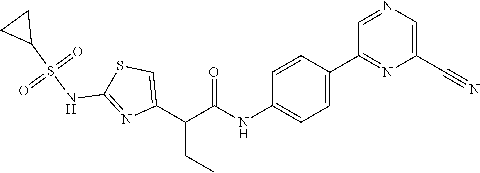 [SMILES: CCC(C(=O)Nc1ccc(-c2cncc(C#N)n2)cc1)c1csc(NS(=O)(=O)C2CC2)n1] |
| T80 | Single enantiomer— |
| | stereochemistry unassigned |
| | 2-(2- |
| | (cyclopropanesulfonamido)thiazol-4- |
| | yl)-N-(4-(6-(trifluoromethyl)pyrazin-2- |
| | yl)phenyl)butanamide |
| | 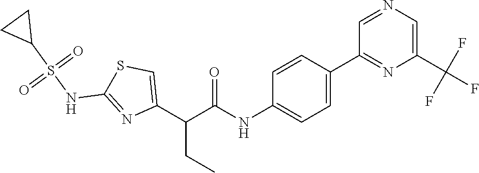 [SMILES: CCC(C(=O)Nc1ccc(-c2cncc(C(F)(F)F)n2)cc1)c1csc(NS(=O)(=O)C2CC2)n1] |
| T81 | 2-(2- |
| | (cyclopropanesulfonamido)thiazol-4- |
| | yl)-N-(4-(6-methoxypyrazin-2- |
| | yl)phenyl)acetamide |
| | 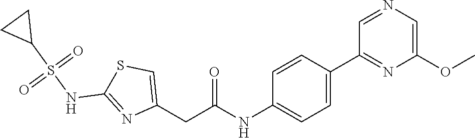 [SMILES: COc1cncc(-c2ccc(NC(=O)Cc3csc(NS(=O)(=O)C4CC4)n3)cc2)n1] |
| T82 | 2-(2- |
| | (cyclopropanesulfonamido)thiazol-4- |
| | yl)-N-(4-(pyrazin-2- |
| | yl)phenyl)acetamide |
| | 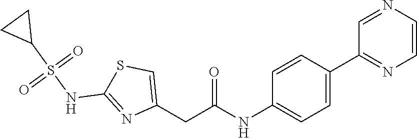 [SMILES: O=C(Cc1csc(NS(=O)(=O)C2CC2)n1)Nc1ccc(-c2cnccn2)cc1] |
| T83 | N-([1,1′-biphenyl]-4-yl)-2- |
| | (cyclopropanesulfonamido)-4,5,6,7- |
| | tetrahydrobenzo[d]thiazole-4- |
| | carboxamide |
| | 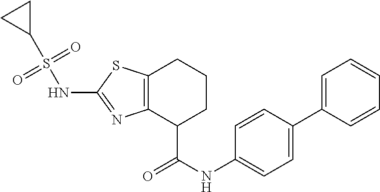 [SMILES: O=C(Nc1ccc(-c2ccccc2)cc1)C1CCCc2sc(NS(=O)(=O)C3CC3)nc21] |
| T84 | 2-(cyclopropanesulfonamido)-N-(4- |
| | (pyridin-3-yl)phenyl)-4,5,6,7- |
| | tetrahydrobenzo[d]thiazole-4- |
| | carboxamide |
| | 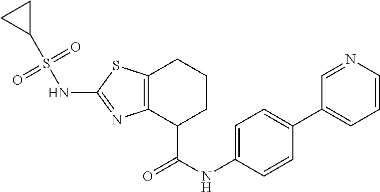 [SMILES: O=C(Nc1ccc(-c2cccnc2)cc1)C1CCCc2sc(NS(=O)(=O)C3CC3)nc21] |
| T85 | N-([1,1′-biphenyl]-4-yl)-2-(2- |
| | (cyclopropanesulfonamido)thiazol-4- |
| | yl)butanamide |
| | 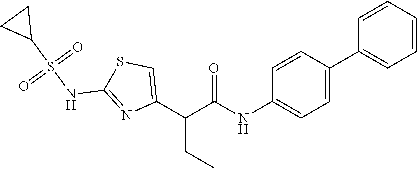 [SMILES: CCC(C(=O)Nc1ccc(-c2ccccc2)cc1)c1csc(NS(=O)(=O)C2CC2)n1] |
| T86 | N-([1,1′-biphenyl]-4-yl)-2-(2- |
| | (cyclopropanesulfonamido)thiazol-4- |
| | yl)-3-methylbutanamide |
| | 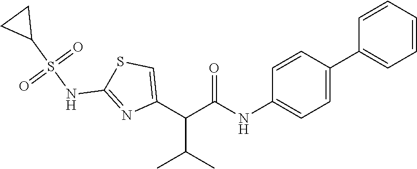 [SMILES: CC(C)C(C(=O)Nc1ccc(-c2ccccc2)cc1)c1csc(NS(=O)(=O)C2CC2)n1] |
| T87 | N-(3′-chloro-[1,1′-biphenyl]-4-yl)-2-(2- |
| | (cyclopropanesulfonamido)thiazol-4- |
| | yl)-2-methylpropanamide |
| | 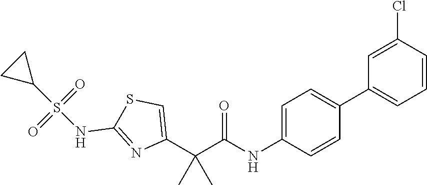 [SMILES: CC(C)(C(=O)Nc1ccc(-c2cccc(Cl)c2)cc1)c1csc(NS(=O)(=O)C2CC2)n1] |
| T88 | N-(3′-cyano-[1,1′-biphenyl]-4-yl)-2-(2- |
| | (cyclopropanesulfonamido)thiazol-4- |
| | yl)-2-methylpropanamide |
| | 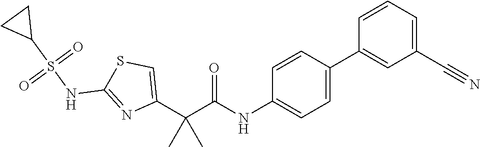 [SMILES: CC(C)(C(=O)Nc1ccc(-c2cccc(C#N)c2)cc1)c1csc(NS(=O)(=O)C2CC2)n1] |
| T89 | 2-(2- |
| | (cyclopropanesulfonamido)thiazol-4- |
| | yl)-2,2-difluoro-N-(4-(pyridin-3- |
| | yl)phenyl)acetamide |
| | 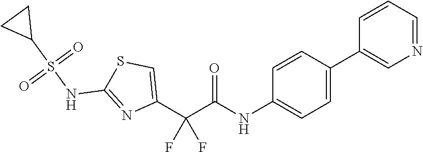 [SMILES: O=C(Nc1ccc(-c2cccnc2)cc1)C(F)(F)c1csc(NS(=O)(=O)C2CC2)n1] |
| T90 | N-(4-(5-fluoropyridin-3-yl)phenyl)-2- |
| | (2-(cyclopropanesulfonamido)thiazol- |
| | 4-yl)butanamide |
| | 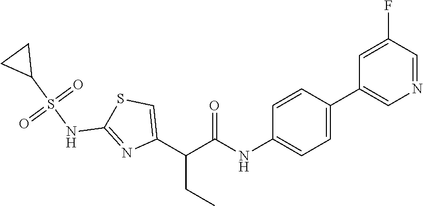 [SMILES: CCC(C(=O)Nc1ccc(-c2cncc(F)c2)cc1)c1csc(NS(=O)(=O)C2CC2)n1] |
| T91 | Single enantiomer— |
| | stereochemistry unassigned |
| | N-(4-(5-fluoropyridin-3-yl)phenyl)-2- |
| | (2-(cyclopropanesulfonamido)thiazol- |
| | 4-yl)butanamide. HCl |
| | 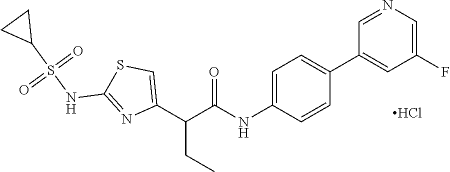 [SMILES: CCC(C(=O)Nc1ccc(-c2cncc(F)c2)cc1)c1csc(NS(=O)(=O)C2CC2)n1 ; Cl] |
| T92 | 2-(2- |
| | (cyclopropanesulfonamido)thiazol-4- |
| | yl)-2-ethyl-N-(4-(pyridin-3- |
| | yl)phenyl)butanamide |
| | 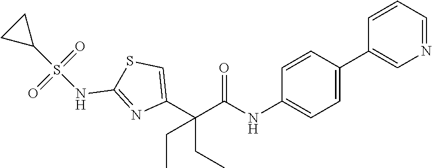 [SMILES: CCC(CC)(C(=O)Nc1ccc(-c2cccnc2)cc1)c1csc(NS(=O)(=O)C2CC2)n1] |
| T93 | N-(4-(5-cyanopyridin-3-yl)phenyl)-2- |
| | (2-(cyclopropanesulfonamido)thiazol- |
| | 4-yl)-2-ethylbutanamide |
| | 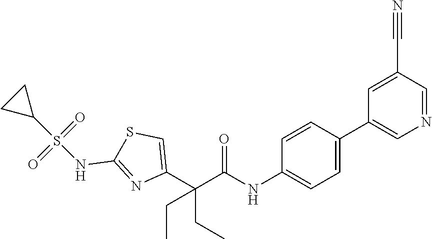 [SMILES: CCC(CC)(C(=O)Nc1ccc(-c2cncc(C#N)c2)cc1)c1csc(NS(=O)(=O)C2CC2)n1] |
| T94 | 2-(2- |
| | (cyclopropanesulfonamido)thiazol-4- |
| | yl)-N-(4-(5-ethoxypyridin-3- |
| | yl)phenyl)propanamide |
| | 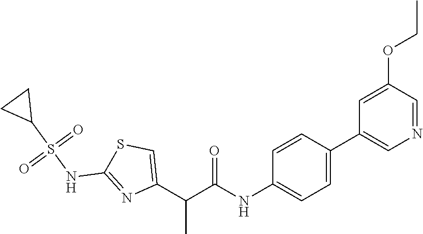 [SMILES: CCOc1cncc(-c2ccc(NC(=O)C(C)c3csc(NS(=O)(=O)C4CC4)n3)cc2)c1] |
| T95 | N-(4-(5-chloropyridin-3-yl)phenyl)-2- |
| | (2-(cyclopropanesulfonamido)thiazol- |
| | 4-yl)propanamide |
| | 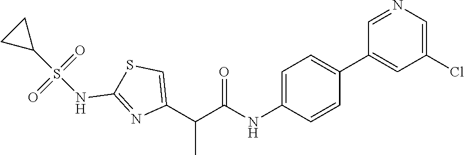 [SMILES: CC(C(=O)Nc1ccc(-c2cncc(Cl)c2)cc1)c1csc(NS(=O)(=O)C2CC2)n1] |
| T96 | 2-(2- |
| | (cyclopropanesulfonamido)thiazol-4- |
| | yl)-N-(4-(5-ethoxypyridin-3- |
| | yl)phenyl)butanamide |
| | 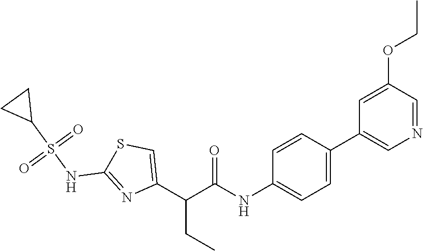 [SMILES: CCOc1cncc(-c2ccc(NC(=O)C(CC)c3csc(NS(=O)(=O)C4CC4)n3)cc2)c1] |
| T97 | N-([1,1′-biphenyl]-4-yl)-2-(2- |
| | (cyclopropanesulfonamido)thiazol-4- |
| | yl)-2-methylpropanamide |
| | 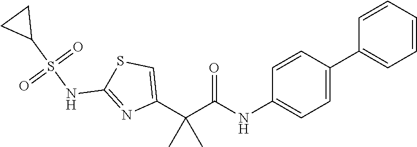 [SMILES: CC(C)(C(=O)Nc1ccc(-c2ccccc2)cc1)c1csc(NS(=O)(=O)C2CC2)n1] |
| T98 | 2-(2- |
| | (cyclopropanesulfonamido)thiazol-4- |
| | yl)-N-(4-(4-methylpyridin-3- |
| | yl)phenyl)acetamide |
| | 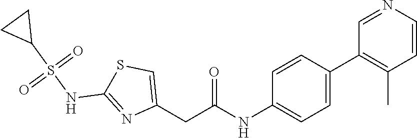 [SMILES: Cc1ccncc1-c1ccc(NC(=O)Cc2csc(NS(=O)(=O)C3CC3)n2)cc1] |
| T99 | 2-(2- |
| | (cyclopropanesulfonamido)thiazol-4- |
| | yl)-N-(4-(5-methylpyridin-3- |
| | yl)phenyl)acetamide |
| | 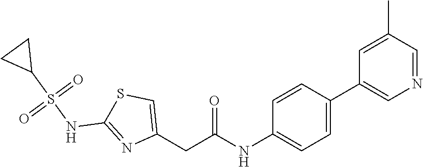 [SMILES: Cc1cncc(-c2ccc(NC(=O)Cc3csc(NS(=O)(=O)C4CC4)n3)cc2)c1] |
| T100 | 2-(2- |
| | (cyclopropanesulfonamido)thiazol-4- |
| | yl)-N-(4-(2-methylpyridin-3- |
| | yl)phenyl)acetamide |
| | 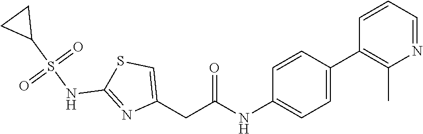 [SMILES: Cc1ncccc1-c1ccc(NC(=O)Cc2csc(NS(=O)(=O)C3CC3)n2)cc1] |
| T101 | 2-(2- |
| | (cyclopropanesulfonamido)thiazol-4- |
| | yl)-N-(4-(6-methylpyridin-3- |
| | yl)phenyl)acetamide |
| | 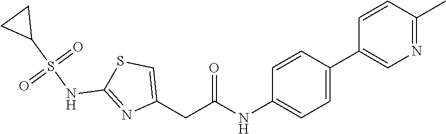 [SMILES: Cc1ccc(-c2ccc(NC(=O)Cc3csc(NS(=O)(=O)C4CC4)n3)cc2)cn1] |
| T102 | 2-(2- |
| | (cyclopropanesulfonamido)thiazol-4- |
| | yl)-N-(4-(pyridin-3- |
| | yl)phenyl)propanamide |
| | 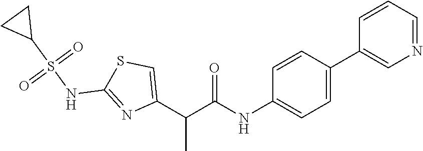 [SMILES: CC(C(=O)Nc1ccc(-c2cccnc2)cc1)c1csc(NS(=O)(=O)C2CC2)n1] |
| T103 | 2-(2- |
| | (cyclopropanesulfonamido)thiazol-4- |
| | yl)-2-methyl-N-(4-(2-methylpyridin-3- |
| | yl)phenyl)propanamide |
| | 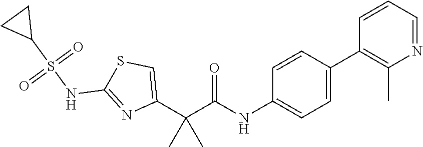 [SMILES: Cc1ncccc1-c1ccc(NC(=O)C(C)(C)c2csc(NS(=O)(=O)C3CC3)n2)cc1] |
| T104 | 2-(2- |
| | (cyclopropanesulfonamido)thiazol-4- |
| | yl)-2-oxo-N-(4-(pyridin-3- |
| | yl)phenyl)acetamide |
| | 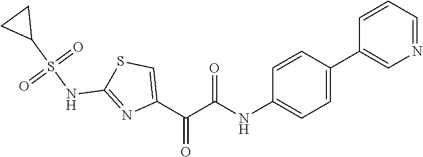 [SMILES: O=C(Nc1ccc(-c2cccnc2)cc1)C(=O)c1csc(NS(=O)(=O)C2CC2)n1] |
| T105 | 2-(2- |
| | (cyclopropanesulfonamido)thiazol-4- |
| | yl)-2-methyl-N-(4-(6-methylpyridin-3- |
| | yl)phenyl)propanamide |
| | 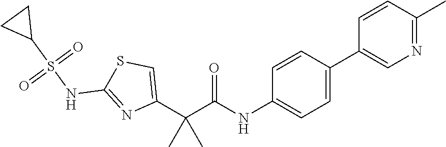 [SMILES: Cc1ccc(-c2ccc(NC(=O)C(C)(C)c3csc(NS(=O)(=O)C4CC4)n3)cc2)cn1] |
| T106 | Single enantiomer— |
| | stereochemistry unassigned |
| | 2-(2- |
| | (cyclopropanesulfonamido)thiazol-4- |
| | yl)-N-(4-(6-(trifluoromethyl)pyrazin-2- |
| | yl)phenyl)butanamide |
| | 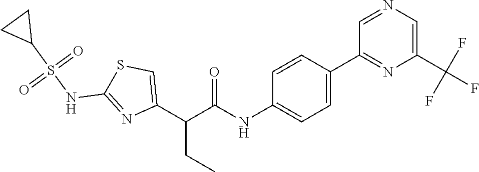 [SMILES: CCC(C(=O)Nc1ccc(-c2cncc(C(F)(F)F)n2)cc1)c1csc(NS(=O)(=O)C2CC2)n1] |
| T107 | Single enantiomer— |
| | stereochemistry unassigned |
| | 2-(2- |
| | (cyclopropanesulfonamido)thiazol-4- |
| | yl)-2-methoxy-N-(4-(6- |
| | methoxypyrazin-2- |
| | yl)phenyl)acetamide |
| | 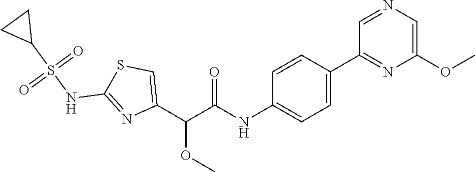 [SMILES: COc1cncc(-c2ccc(NC(=O)C(OC)c3csc(NS(=O)(=O)C4CC4)n3)cc2)n1] |
| T108 | Single enantiomer— |
| | stereochemistry unassigned |
| | 2-(2- |
| | (cyclopropanesulfonamido)thiazol-4- |
| | yl)-2-methoxy-N-(4-(6- |
| | methoxypyrazin-2- |
| | yl)phenyl)acetamide |
| | 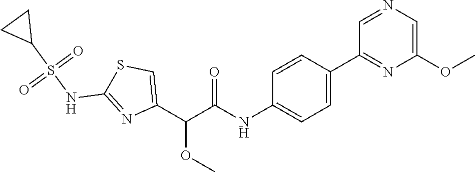 [SMILES: COc1cncc(-c2ccc(NC(=O)C(OC)c3csc(NS(=O)(=O)C4CC4)n3)cc2)n1] |
| T109 | 2-(2- |
| | (cyclopropanesulfonamido)thiazol-4- |
| | yl)-2-methyl-N-(5-(6-(2,2,2- |
| | trifluoroethoxy)pyrazin-2-yl)pyridin-2- |
| | yl)propanamide |
| | 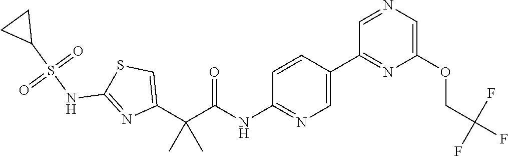 [SMILES: CC(C)(C(=O)Nc1ccc(-c2cncc(OCC(F)(F)F)n2)cn1)c1csc(NS(=O)(=O)C2CC2)n1] |
| T110 | 2-(2- |
| | (cyclopropanesulfonamido)thiazol-4- |
| | yl)-N-(3-fluoro-5-(pyrazin-2-yl)pyridin- |
| | 2-yl)-2-methylpropanamide |
| | 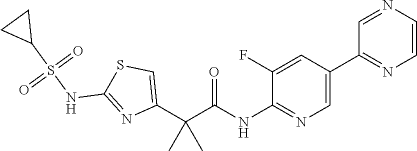 [SMILES: CC(C)(C(=O)Nc1ncc(-c2cnccn2)cc1F)c1csc(NS(=O)(=O)C2CC2)n1] |
| T111 | 2-(2- |
| | (cyclopropanesulfonamido)thiazol-4- |
| | yl)-N-(5-(6-ethoxypyrazin-2-yl)-3- |
| | fluoropyridin-2-yl)-2- |
| | methylpropanamide |
| | 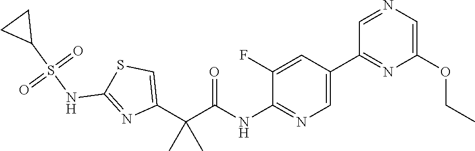 [SMILES: CCOc1cncc(-c2cnc(NC(=O)C(C)(C)c3csc(NS(=O)(=O)C4CC4)n3)c(F)c2)n1] |
| T112 | 2-(2- |
| | (cyclopropanesulfonamido)thiazol-4- |
| | yl)-N-(3-fluoro-5-(6- |
| | (trifluoromethyl)pyrazin-2-yl)pyridin-2- |
| | yl)-2-methylpropanamide |
| | 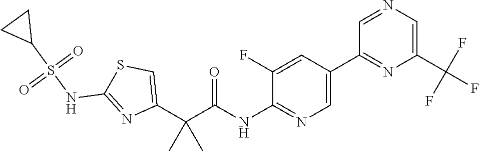 [SMILES: CC(C)(C(=O)Nc1ncc(-c2cncc(C(F)(F)F)n2)cc1F)c1csc(NS(=O)(=O)C2CC2)n1] |
| T113 | N-(5-(6-cyanopyrazin-2-yl)-3- |
| | fluoropyridin-2-yl)-2-(2- |
| | (cyclopropanesulfonamido)thiazol-4- |
| | yl)-2-methylpropanamide |
| | 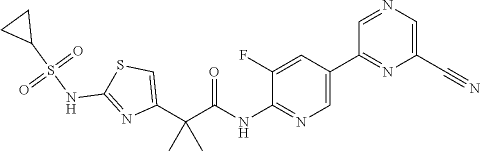 [SMILES: CC(C)(C(=O)Nc1ncc(-c2cncc(C#N)n2)cc1F)c1csc(NS(=O)(=O)C2CC2)n1] |
| T114 | 2-(2- |
| | (cyclopropanesulfonamido)thiazol-4- |
| | yl)-2-methyl-N-(5′-(2,2,2- |
| | trifluoroethoxy)-[3,3′-bipyridin]-6- |
| | yl)propanamide |
| | 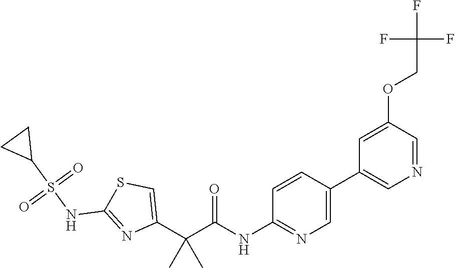 [SMILES: CC(C)(C(=O)Nc1ccc(-c2cncc(OCC(F)(F)F)c2)cn1)c1csc(NS(=O)(=O)C2CC2)n1] |
| T115 | 2-(2- |
| | (cyclopropanesulfonamido)thiazol-4- |
| | yl)-N-(5′-(difluoromethoxy)-[3,3′- |
| | bipyridin]-6-yl)-2-methylpropanamide |
| | 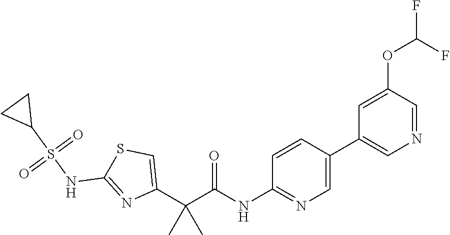 [SMILES: CC(C)(C(=O)Nc1ccc(-c2cncc(OC(F)F)c2)cn1)c1csc(NS(=O)(=O)C2CC2)n1] |
| T116 | N-([2,3′-bipyridin]-5-yl)-2-(2- |
| | (cyclopropanesulfonamido)thiazol-4- |
| | yl)-2-methylpropanamide |
| | 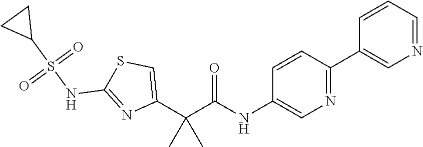 [SMILES: CC(C)(C(=O)Nc1ccc(-c2cccnc2)nc1)c1csc(NS(=O)(=O)C2CC2)n1] |
| T117 | 2-(2- |
| | (cyclopropanesulfonamido)thiazol-4- |
| | yl)-2-methyl-N-(6-(pyrimidin-5- |
| | yl)pyridin-3-yl)propanamide |
| | 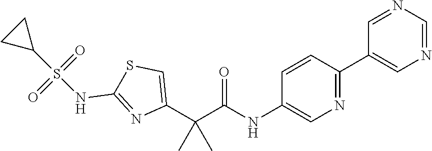 [SMILES: CC(C)(C(=O)Nc1ccc(-c2cncnc2)nc1)c1csc(NS(=O)(=O)C2CC2)n1] |
| T118 | 2-(2- |
| | (cyclopropanesulfonamido)thiazol-4- |
| | yl)-N-(4-(5-(difluoromethoxy)pyridin- |
| | 3-yl)phenyl)-2-methylpropanamide |
| | 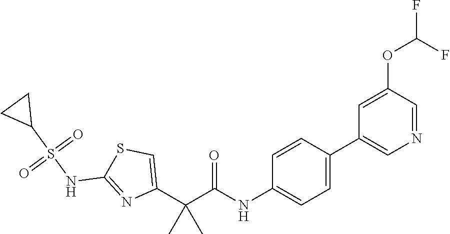 [SMILES: CC(C)(C(=O)Nc1ccc(-c2cncc(OC(F)F)c2)cc1)c1csc(NS(=O)(=O)C2CC2)n1] |
| T119 | 2-(2- |
| | (cyclopropanesulfonamido)thiazol-4- |
| | yl)-2-ethyl-N-(4-(6-methoxypyrazin-2- |
| | yl)phenyl)butanamide |
| | 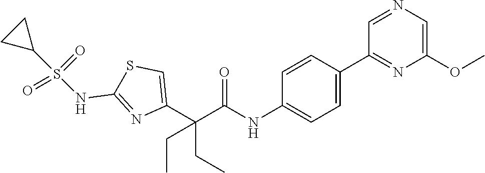 [SMILES: CCC(CC)(C(=O)Nc1ccc(-c2cncc(OC)n2)cc1)c1csc(NS(=O)(=O)C2CC2)n1] |
| T120 | N-(4-(5-chloropyridin-3-yl)phenyl)-2- |
| | (2-(cyclopropanesulfonamido)thiazol- |
| | 4-yl)-2-ethylbutanamide |
| | 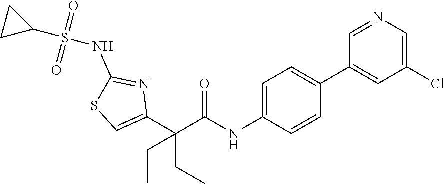 [SMILES: CCC(CC)(C(=O)Nc1ccc(-c2cncc(Cl)c2)cc1)c1csc(NS(=O)(=O)C2CC2)n1] |
| T121 | 2-(2- |
| | (cyclopropanesulfonamido)thiazol-4- |
| | yl)-2-ethyl-N-(2-fluoro-4-(pyridin-3- |
| | yl)phenyl)butanamide |
| | 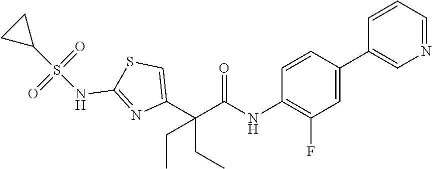 [SMILES: CCC(CC)(C(=O)Nc1ccc(-c2cccnc2)cc1F)c1csc(NS(=O)(=O)C2CC2)n1] |
| T122 | 2-(2- |
| | (cyclopropanesulfonamido)thiazol-4- |
| | yl)-2-ethyl-N-(4-(pyrazin-2- |
| | yl)phenyl)butanamide |
| | 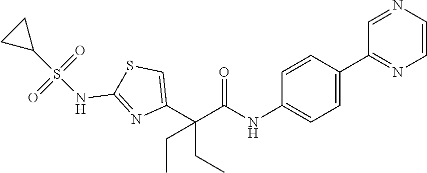 [SMILES: CCC(CC)(C(=O)Nc1ccc(-c2cnccn2)cc1)c1csc(NS(=O)(=O)C2CC2)n1] |
| T123 | 2-(2- |
| | (cyclopropanesulfonamido)thiazol-4- |
| | yl)-2-ethyl-N-(4-(6-propoxypyrazin-2- |
| | yl)phenyl)butanamide |
| | 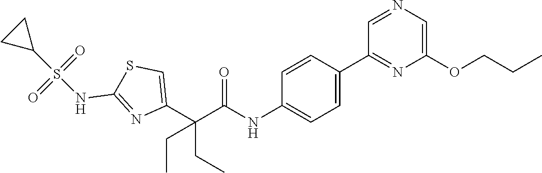 [SMILES: CCCOc1cncc(-c2ccc(NC(=O)C(CC)(CC)c3csc(NS(=O)(=O)C4CC4)n3)cc2)n1] |
| T124 | 2-(2- |
| | (cyclopropanesulfonamido)thiazol-4- |
| | yl)-N-(2-fluoro-4-(6- |
| | (trifluoromethyl)pyrazin-2-yl)phenyl)- |
| | 2-methylpropanamide |
| | 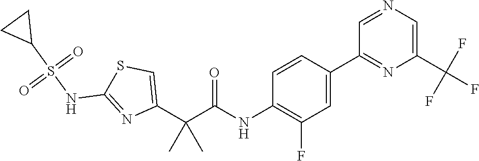 [SMILES: CC(C)(C(=O)Nc1ccc(-c2cncc(C(F)(F)F)n2)cc1F)c1csc(NS(=O)(=O)C2CC2)n1] |
| T125 | 2-(2- |
| | (cyclopropanesulfonamido)thiazol-4- |
| | yl)-N-(4-(5-ethoxypyridin-3-yl)-2- |
| | fluorophenyl)-2-methylpropanamide |
| | 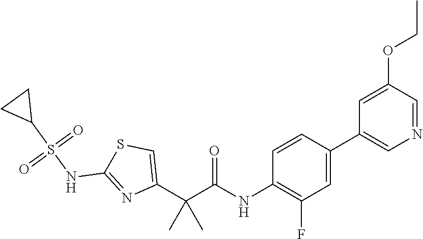 [SMILES: CCOc1cncc(-c2ccc(NC(=O)C(C)(C)c3csc(NS(=O)(=O)C4CC4)n3)c(F)c2)c1] |
| T126 | 2-(2- |
| | (cyclopropanesulfonamido)thiazol-4- |
| | yl)-N-(2-fluoro-4-(5-fluoropyridin-3- |
| | yl)phenyl)-2-methylpropanamide |
| | 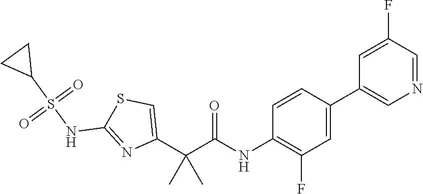 [SMILES: CC(C)(C(=O)Nc1ccc(-c2cncc(F)c2)cc1F)c1csc(NS(=O)(=O)C2CC2)n1] |
| T127 | 2-(2- |
| | (cyclopropanesulfonamido)thiazol-4- |
| | yl)-2-methyl-N-(4-(6-(2,2,2- |
| | trifluoroethoxy)pyrazin-2- |
| | yl)phenyl)propanamide |
| | 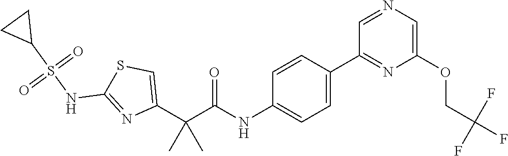 [SMILES: CC(C)(C(=O)Nc1ccc(-c2cncc(OCC(F)(F)F)n2)cc1)c1csc(NS(=O)(=O)C2CC2)n1] |
| T128 | N-(4-(5-chloropyridin-3-yl)-2- |
| | fluorophenyl)-2-(2- |
| | (cyclopropanesulfonamido)thiazol-4- |
| | yl)-2-methylpropanamide |
| | 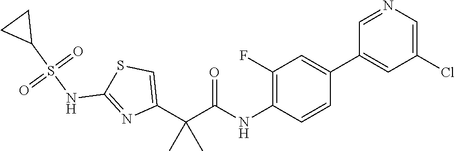 [SMILES: CC(C)(C(=O)Nc1ccc(-c2cncc(Cl)c2)cc1F)c1csc(NS(=O)(=O)C2CC2)n1] |
| T129 | N-(4-(5-cyanopyridin-3-yl)-2- |
| | fluorophenyl)-2-(2- |
| | (cyclopropanesulfonamido)thiazol-4- |
| | yl)-2-methylpropanamide |
| | 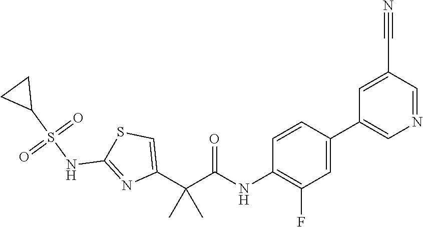 [SMILES: CC(C)(C(=O)Nc1ccc(-c2cncc(C#N)c2)cc1F)c1csc(NS(=O)(=O)C2CC2)n1] |
| T130 | 1-(2- |
| | (cyclopropanesulfonamido)thiazol-4- |
| | yl)-N-(4-(6-methoxypyrazin-2- |
| | yl)phenyl)cyclopentane-1- |
| | carboxamide |
| | 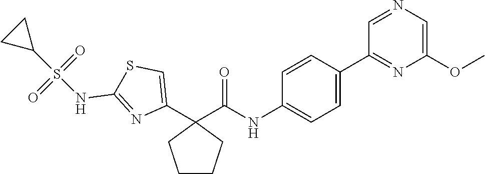 [SMILES: COc1cncc(-c2ccc(NC(=O)C3(c4csc(NS(=O)(=O)C5CC5)n4)CCCC3)cc2)n1] |
| T131 | 2-(2- |
| | (cyclopropanesulfonamido)thiazol-4- |
| | yl)-N-(2-fluoro-4-(6-(2,2,2- |
| | trifluoroethoxy)pyrazin-2-yl)phenyl)-2- |
| | methylpropanamide |
| | 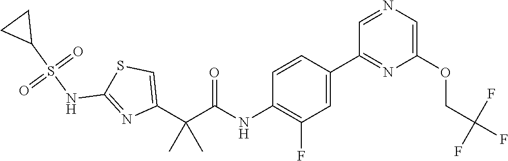 [SMILES: CC(C)(C(=O)Nc1ccc(-c2cncc(OCC(F)(F)F)n2)cc1F)c1csc(NS(=O)(=O)C2CC2)n1] |
| T132 | 2-(2- |
| | (cyclopropanesulfonamido)thiazol-4- |
| | yl)-N-(2-fluoro-4-(5-(2,2,2- |
| | trifluoroethoxy)pyridin-3-yl)phenyl)-2- |
| | methylpropanamide |
| | 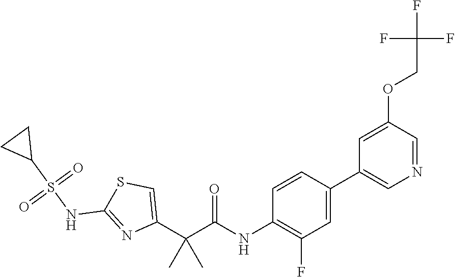 [SMILES: CC(C)(C(=O)Nc1ccc(-c2cncc(OCC(F)(F)F)c2)cc1F)c1csc(NS(=O)(=O)C2CC2)n1] |
| T133 | 2-(2- |
| | (cyclopropanesulfonamido)thiazol-4- |
| | yl)-N-(2-fluoro-4-(5- |
| | (trifluoromethyl)pyridin-3-yl)phenyl)-2- |
| | methylpropanamide |
| | 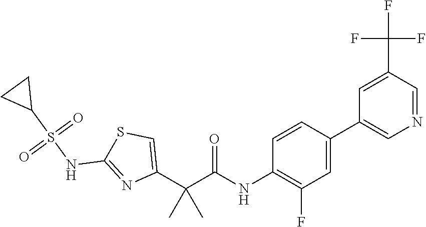 [SMILES: CC(C)(C(=O)Nc1ccc(-c2cncc(C(F)(F)F)c2)cc1F)c1csc(NS(=O)(=O)C2CC2)n1] |
| T134 | 2-(2- |
| | (cyclopropanesulfonamido)thiazol-4- |
| | yl)-N-(4-(6-ethoxypyrazin-2-yl)-2- |
| | fluorophenyl)-2-methylpropanamide |
| | 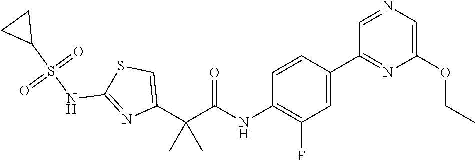 [SMILES: CCOc1cncc(-c2ccc(NC(=O)C(C)(C)c3csc(NS(=O)(=O)C4CC4)n3)c(F)c2)n1] |
| T135 | 2-(2- |
| | (cyclopropanesulfonamido)thiazol-4- |
| | yl)-2-methyl-N-(4-(5-(2,2,2- |
| | trifluoroethoxy)pyridin-3- |
| | yl)phenyl)propanamide |
| | 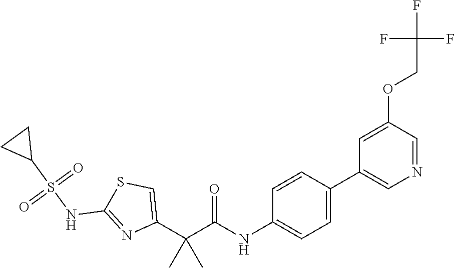 [SMILES: CC(C)(C(=O)Nc1ccc(-c2cncc(OCC(F)(F)F)c2)cc1)c1csc(NS(=O)(=O)C2CC2)n1] |
| T136 | 2-(2- |
| | (cyclopropanesulfonamido)thiazol-4- |
| | yl)-N-(4-(6-ethynylpyrazin-2- |
| | yl)phenyl)-2-methylpropanamide |
| | 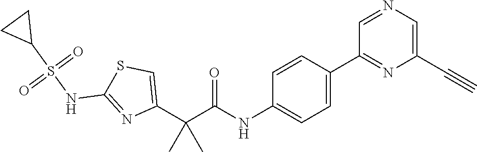 [SMILES: C#Cc1cncc(-c2ccc(NC(=O)C(C)(C)c3csc(NS(=O)(=O)C4CC4)n3)cc2)n1] |
| T137 | 2-(2- |
| | (cyclopropanesulfonamido)thiazol-4- |
| | yl)-N-(4-(6-ethoxypyrazin-2-yl)-2- |
| | methylphenyl)-2-methylpropanamide |
| | 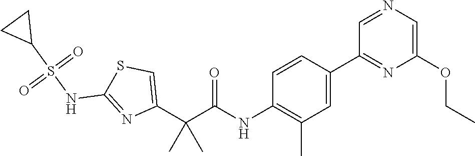 [SMILES: CCOc1cncc(-c2ccc(NC(=O)C(C)(C)c3csc(NS(=O)(=O)C4CC4)n3)c(C)c2)n1] |
| T138 | N-(4-(6-chloropyrazin-2-yl)-2- |
| | methylphenyl)-2-(2- |
| | (cyclopropanesulfonamido)thiazol-4- |
| | yl)-2-methylpropanamide |
| | 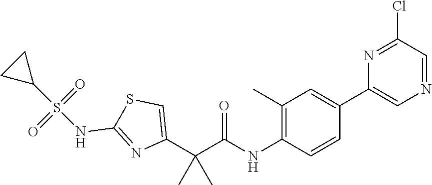 [SMILES: Cc1cc(-c2cncc(Cl)n2)ccc1NC(=O)C(C)(C)c1csc(NS(=O)(=O)C2CC2)n1] |
| T139 | 2-(2- |
| | (cyclopropanesulfonamido)thiazol-4- |
| | yl)-N-(4-(5-(difluoromethoxy)pyridin- |
| | 3-yl)-2-fluorophenyl)-2- |
| | methylpropanamide |
| | 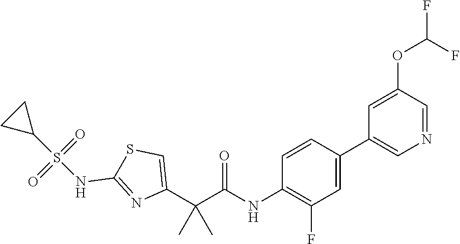 [SMILES: CC(C)(C(=O)Nc1ccc(-c2cncc(OC(F)F)c2)cc1F)c1csc(NS(=O)(=O)C2CC2)n1] |
| T140 | 2-(2- |
| | (cyclopropanesulfonamido)thiazol-4- |
| | yl)-2-methyl-N-(5-(pyrazin-2- |
| | yl)pyridin-2-yl)propanamide |
| | 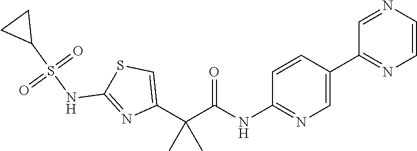 [SMILES: CC(C)(C(=O)Nc1ccc(-c2cnccn2)cn1)c1csc(NS(=O)(=O)C2CC2)n1] |
| T141 | N-(5-(6-cyclobutoxypyrazin-2- |
| | yl)pyridin-2-yl)-2-(2- |
| | (cyclopropanesulfonamido)thiazol-4- |
| | yl)-2-methylpropanamide |
| | 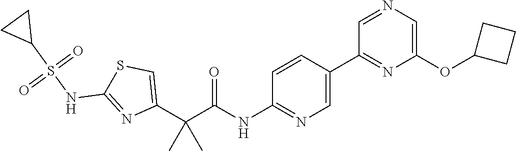 [SMILES: CC(C)(C(=O)Nc1ccc(-c2cncc(OC3CCC3)n2)cn1)c1csc(NS(=O)(=O)C2CC2)n1] |
| T142 | 2-(2- |
| | (cyclopropanesulfonamido)thiazol-4- |
| | yl)-N-(5-(6-cyclopropoxypyrazin-2- |
| | yl)pyridin-2-yl)-2-methylpropanamide |
| | 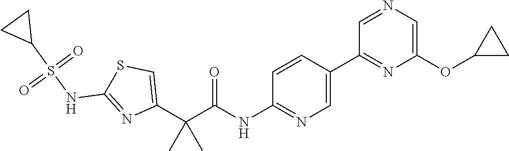 [SMILES: CC(C)(C(=O)Nc1ccc(-c2cncc(OC3CC3)n2)cn1)c1csc(NS(=O)(=O)C2CC2)n1] |
| T143 | 2-(2- |
| | (cyclopropanesulfonamido)thiazol-4- |
| | yl)-N-(5-(6-isopropoxypyrazin-2- |
| | yl)pyridin-2-yl)-2-methylpropanamide |
| | 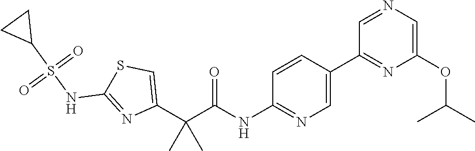 [SMILES: CC(C)Oc1cncc(-c2ccc(NC(=O)C(C)(C)c3csc(NS(=O)(=O)C4CC4)n3)nc2)n1] |
| T144 | N-([3,3′-bipyridin]-6-yl)-2-(2- |
| | (cyclopropanesulfonamido)thiazol-4- |
| | yl)-2-ethylbutanamide |
| | 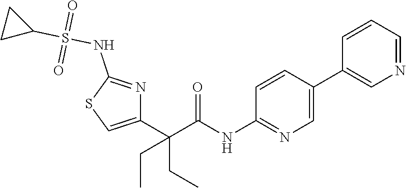 [SMILES: CCC(CC)(C(=O)Nc1ccc(-c2cccnc2)cn1)c1csc(NS(=O)(=O)C2CC2)n1] |
| T145 | 2-(2- |
| | (cyclopropanesulfonamido)thiazol-4- |
| | yl)-N-(5′-ethoxy-[3,3′-bipyridin]-6-yl)- |
| | 2-ethylbutanamide |
| | 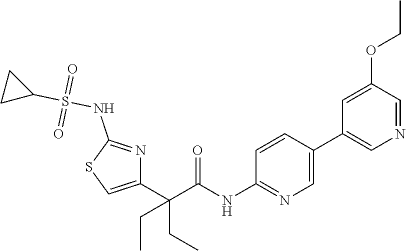 [SMILES: CCOc1cncc(-c2ccc(NC(=O)C(CC)(CC)c3csc(NS(=O)(=O)C4CC4)n3)nc2)c1] |
| T146 | 2-(2- |
| | (cyclopropanesulfonamido)thiazol-4- |
| | yl)-2-methyl-N-(5′-propoxy-[3,3′- |
| | bipyridin]-6-yl)propanamide |
| | 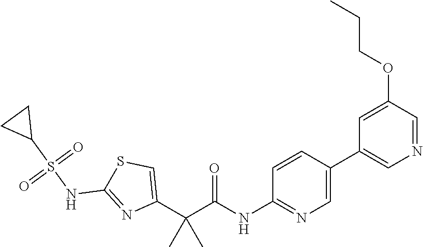 [SMILES: CCCOc1cncc(-c2ccc(NC(=O)C(C)(C)c3csc(NS(=O)(=O)C4CC4)n3)nc2)c1] |
| T147 | 2-(2- |
| | (cyclopropanesulfonamido)thiazol-4- |
| | yl)-2-ethyl-N-(5-(6- |
| | (trifluoromethyl)pyrazin-2-yl)pyridin-2- |
| | yl)butanamide |
| | 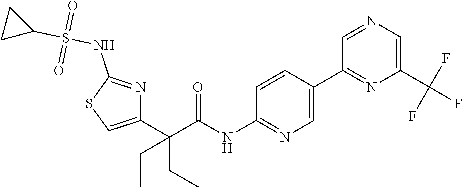 [SMILES: CCC(CC)(C(=O)Nc1ccc(-c2cncc(C(F)(F)F)n2)cn1)c1csc(NS(=O)(=O)C2CC2)n1] |
| T148 | N-([3,3′-bipyridin]-6-yl)-2-(2- |
| | (cyclopropanesulfonamido)thiazol-4- |
| | yl)-2-methylpropanamide |
| | 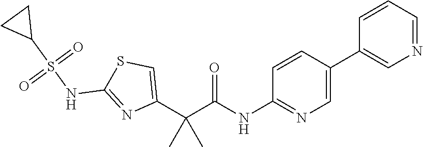 [SMILES: CC(C)(C(=O)Nc1ccc(-c2cccnc2)cn1)c1csc(NS(=O)(=O)C2CC2)n1] |
| T149 | 2-(2- |
| | (cyclopropanesulfonamido)thiazol-4- |
| | yl)-N-(2-methoxy-4-(pyridin-3- |
| | yl)phenyl)-2-methylpropanamide |
| | 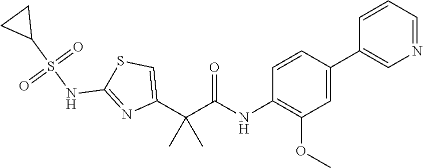 [SMILES: COc1cc(-c2cccnc2)ccc1NC(=O)C(C)(C)c1csc(NS(=O)(=O)C2CC2)n1] |
| T150 | 2-(2- |
| | (cyclopropanesulfonamido)thiazol-4- |
| | yl)-N-(3-methoxy-4-(pyridin-3- |
| | yl)phenyl)-2-methylpropanamide |
| | 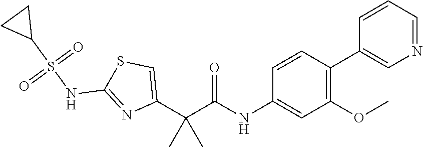 [SMILES: COc1cc(NC(=O)C(C)(C)c2csc(NS(=O)(=O)C3CC3)n2)ccc1-c1cccnc1] |
| T151 | 2-(2- |
| | (cyclopropanesulfonamido)thiazol-4- |
| | yl)-N-(3-fluoro-4-(pyridin-3-yl)phenyl)- |
| | 2-methylpropanamide |
| | 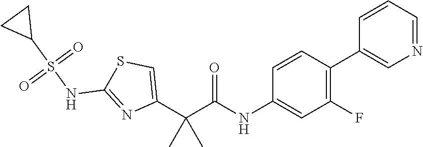 [SMILES: CC(C)(C(=O)Nc1ccc(-c2cccnc2)c(F)c1)c1csc(NS(=O)(=O)C2CC2)n1] |
| T152 | N-(3-cyano-4-(pyridin-3-yl)phenyl)-2- |
| | (2-(cyclopropanesulfonamido)thiazol- |
| | 4-yl)-2-methylpropanamide |
| | 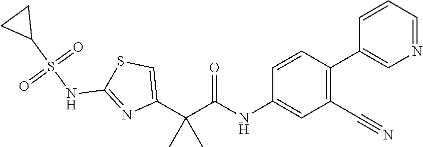 [SMILES: CC(C)(C(=O)Nc1ccc(-c2cccnc2)c(C#N)c1)c1csc(NS(=O)(=O)C2CC2)n1] |
| T153 | N-(3-chloro-4-(pyridin-3-yl)phenyl)-2- |
| | (2-(cyclopropanesulfonamido)thiazol- |
| | 4-yl)-2-methylpropanamide |
| | 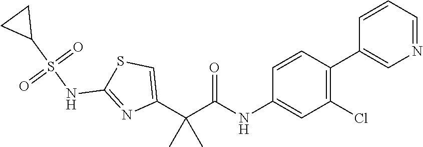 [SMILES: CC(C)(C(=O)Nc1ccc(-c2cccnc2)c(Cl)c1)c1csc(NS(=O)(=O)C2CC2)n1] |
| T154 | N-(4-(6-cyanopyrazin-2-yl)phenyl)-2- |
| | (2-(cyclopropanesulfonamido)thiazol- |
| | 4-yl)-2-methylpropanamide |
| | 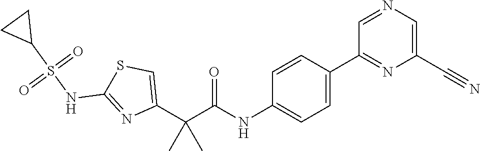 [SMILES: CC(C)(C(=O)Nc1ccc(-c2cncc(C#N)n2)cc1)c1csc(NS(=O)(=O)C2CC2)n1] |
| T155 | N-(4-(6-chloropyrazin-2-yl)phenyl)-2- |
| | (2-(cyclopropanesulfonamido)thiazol- |
| | 4-yl)-2-methylpropanamide |
| | 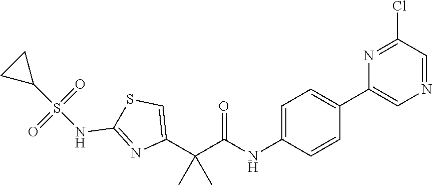 [SMILES: CC(C)(C(=O)Nc1ccc(-c2cncc(Cl)n2)cc1)c1csc(NS(=O)(=O)C2CC2)n1] |
| T156 | 2-(2- |
| | (cyclopropanesulfonamido)thiazol-4- |
| | yl)-2-ethyl-N-(4-(5-fluoropyridin-3- |
| | yl)phenyl)butanamide |
| | 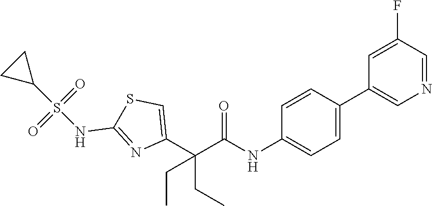 [SMILES: CCC(CC)(C(=O)Nc1ccc(-c2cncc(F)c2)cc1)c1csc(NS(=O)(=O)C2CC2)n1] |
| T157 | 2-(2- |
| | (cyclopropanesulfonamido)thiazol-4- |
| | yl)-2-methyl-N-(4-(5-propoxypyridin- |
| | 3-yl)phenyl)propanamide |
| | 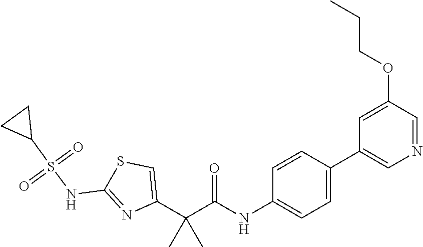 [SMILES: CCCOc1cncc(-c2ccc(NC(=O)C(C)(C)c3csc(NS(=O)(=O)C4CC4)n3)cc2)c1] |
| T158 | 2-(2- |
| | (cyclopropanesulfonamido)thiazol-4- |
| | yl)-N-(4-(5-isopropoxypyridin-3- |
| | yl)phenyl)-2-methylpropanamide |
| | 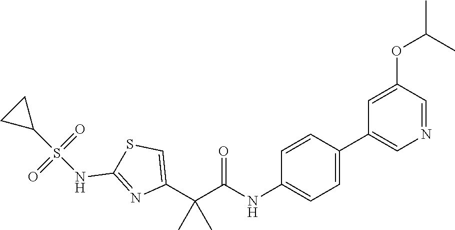 [SMILES: CC(C)Oc1cncc(-c2ccc(NC(=O)C(C)(C)c3csc(NS(=O)(=O)C4CC4)n3)cc2)c1] |
| T159 | 2-(2- |
| | (cyclopropanesulfonamido)thiazol-4- |
| | yl)-N-(2-fluoro-4-(5-isopropoxypyridin- |
| | 3-yl)phenyl)-2-methylpropanamide |
| | 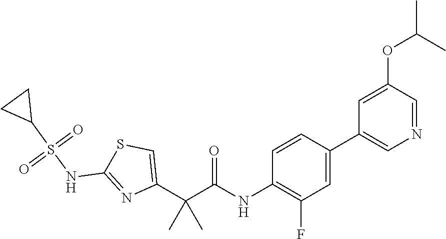 [SMILES: CC(C)Oc1cncc(-c2ccc(NC(=O)C(C)(C)c3csc(NS(=O)(=O)C4CC4)n3)c(F)c2)c1] |
| T160 | N-(4-(6-chloropyrazin-2-yl)phenyl)-2- |
| | (2-(cyclopropanesulfonamido)thiazol- |
| | 4-yl)-2-ethylbutanamide |
| | 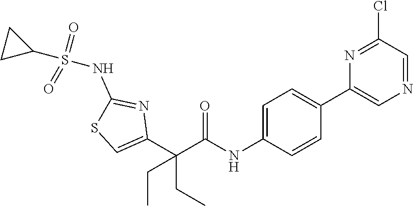 [SMILES: CCC(CC)(C(=O)Nc1ccc(-c2cncc(Cl)n2)cc1)c1csc(NS(=O)(=O)C2CC2)n1] |
| T161 | N-(4-(6-cyanopyrazin-2-yl)phenyl)-2- |
| | (2-(cyclopropanesulfonamido)thiazol- |
| | 4-yl)-2-ethylbutanamide |
| | 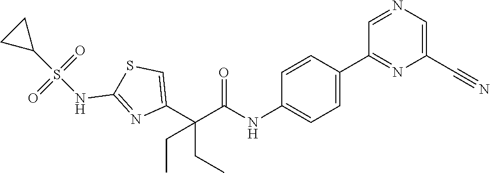 [SMILES: CCC(CC)(C(=O)Nc1ccc(-c2cncc(C#N)n2)cc1)c1csc(NS(=O)(=O)C2CC2)n1] |
| T163 | 2-methyl-2-(2- |
| | (methylsulfonamido)thiazol-4-yl)-N- |
| | (4-(pyridin-3-yl)phenyl)propanamide |
| | 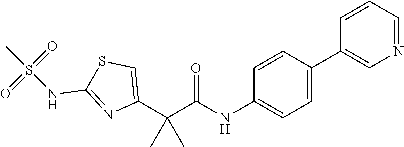 [SMILES: CC(C)(C(=O)Nc1ccc(-c2cccnc2)cc1)c1csc(NS(C)(=O)=O)n1] |
| T164 | 2-(2- |
| | (cyclopropanesulfonamido)thiazol-4- |
| | yl)-N,2-dimethyl-N-(4-(pyridin-3- |
| | yl)phenyl)propanamide |
| | 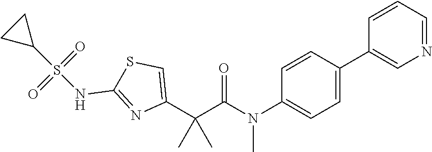 [SMILES: CN(C(=O)C(C)(C)c1csc(NS(=O)(=O)C2CC2)n1)c1ccc(-c2cccnc2)cc1] |
| T169 | 2-(cyclopropanesulfonamido)-N-(5-(6- |
| | (trifluoromethyl)pyrazin-2-yl)pyridin-2- |
| | yl)-5,6-dihydro-4H- |
| | cyclopenta[d]thiazole-4-carboxamide |
| | 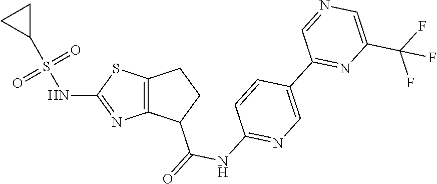 [SMILES: O=C(Nc1ccc(-c2cncc(C(F)(F)F)n2)cn1)C1CCc2sc(NS(=O)(=O)C3CC3)nc21] |
| T170 | 2-(2- |
| | (cyclopropanesulfonamido)thiazol-4- |
| | yl)-4-methoxy-N-(5-(pyrazin-2- |
| | yl)pyridin-2-yl)butanamide |
| | 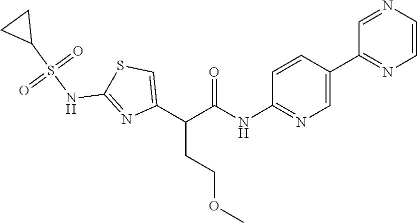 [SMILES: COCCC(C(=O)Nc1ccc(-c2cnccn2)cn1)c1csc(NS(=O)(=O)C2CC2)n1] |
| T171 | 2-(2- |
| | (cyclopropanesulfonamido)thiazol-4- |
| | yl)-4-methoxy-N-(5′-methoxy-[3,3′- |
| | bipyridin]-6-yl)butanamide |
| | 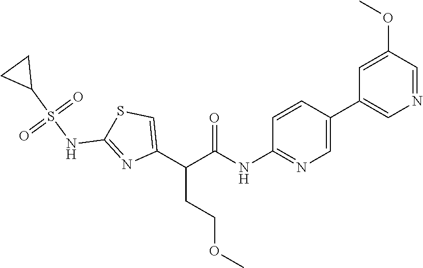 [SMILES: COCCC(C(=O)Nc1ccc(-c2cncc(OC)c2)cn1)c1csc(NS(=O)(=O)C2CC2)n1] |
| T172 | 2-(2- |
| | (cyclopropanesulfonamido)thiazol-4- |
| | yl)-2-isopropoxy-N-(5-(6- |
| | (trifluoromethyl)pyrazin-2-yl)pyridin-2- |
| | yl)acetamide |
| | 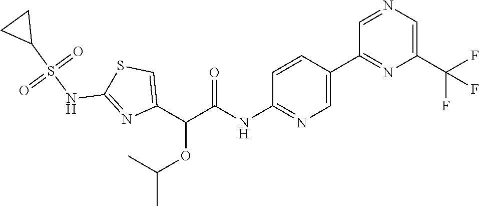 [SMILES: CC(C)OC(C(=O)Nc1ccc(-c2cncc(C(F)(F)F)n2)cn1)c1csc(NS(=O)(=O)C2CC2)n1] |
| T173 | 2-(2- |
| | (cyclopropanesulfonamido)thiazol-4- |
| | yl)-N-(5-(6-propoxypyrazin-2- |
| | yl)pyridin-2-yl)butanamide |
| | 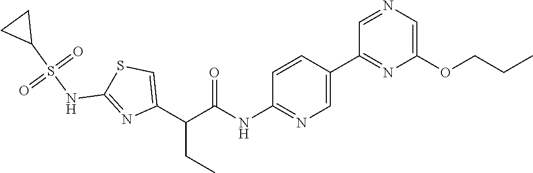 [SMILES: CCCOc1cncc(-c2ccc(NC(=O)C(CC)c3csc(NS(=O)(=O)C4CC4)n3)nc2)n1] |
| T174 | 2-(2- |
| | (cyclopropanesulfonamido)thiazol-4- |
| | yl)-N-(5-(6-isopropoxypyrazin-2- |
| | yl)pyridin-2-yl)butanamide |
| | 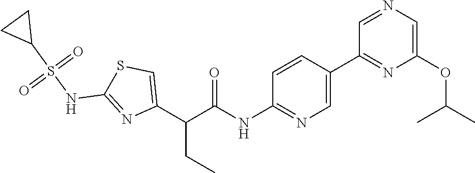 [SMILES: CCC(C(=O)Nc1ccc(-c2cncc(OC(C)C)n2)cn1)c1csc(NS(=O)(=O)C2CC2)n1] |
| T175 | 2-(2- |
| | (cyclopropanesulfonamido)thiazol-4- |
| | yl)-N-(5-(6-(trifluoromethyl)pyrazin-2- |
| | yl)pyridin-2-yl)butanamide |
| | 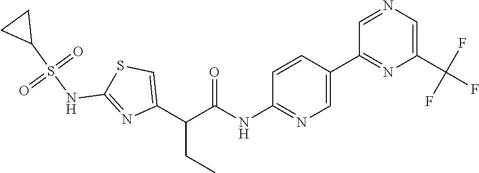 [SMILES: CCC(C(=O)Nc1ccc(-c2cncc(C(F)(F)F)n2)cn1)c1csc(NS(=O)(=O)C2CC2)n1] |
| T176 | 2-(2- |
| | (cyclopropanesulfonamido)thiazol-4- |
| | yl)-N-(5-(6-methoxypyrazin-2- |
| | yl)pyridin-2-yl)butanamide |
| | 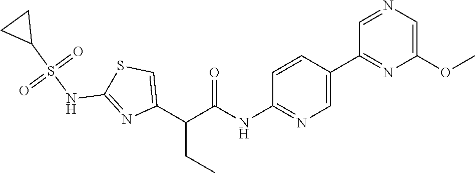 [SMILES: CCC(C(=O)Nc1ccc(-c2cncc(OC)n2)cn1)c1csc(NS(=O)(=O)C2CC2)n1] |
| T177 | 2-(2- |
| | (cyclopropanesulfonamido)thiazol-4- |
| | yl)-N-(5-(6-ethoxypyrazin-2-yl)pyridin- |
| | 2-yl)butanamide |
| | 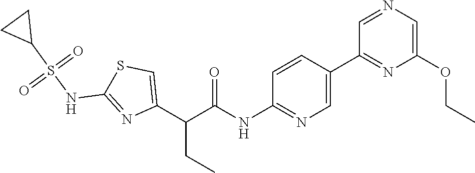 [SMILES: CCOc1cncc(-c2ccc(NC(=O)C(CC)c3csc(NS(=O)(=O)C4CC4)n3)nc2)n1] |
| T178 | N-(5-(6-cyanopyrazin-2-yl)pyridin-2- |
| | yl)-2-(2- |
| | (cyclopropanesulfonamido)thiazol-4- |
| | yl)butanamide |
| | 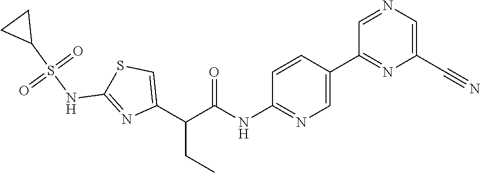 [SMILES: CCC(C(=O)Nc1ccc(-c2cncc(C#N)n2)cn1)c1csc(NS(=O)(=O)C2CC2)n1] |
| T179 | 2-(2- |
| | (cyclopropanesulfonamido)thiazol-4- |
| | yl)-N-(5′-fluoro-[3,3′-bipyridin]-6- |
| | yl)butanamide |
| | 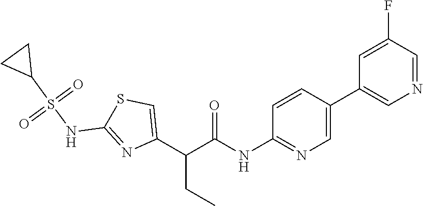 [SMILES: CCC(C(=O)Nc1ccc(-c2cncc(F)c2)cn1)c1csc(NS(=O)(=O)C2CC2)n1] |
| T180 | N-(5′-cyano-[3,3′-bipyridin]-6-yl)-2-(2- |
| | (cyclopropanesulfonamido)thiazol-4- |
| | yl)butanamide |
| | 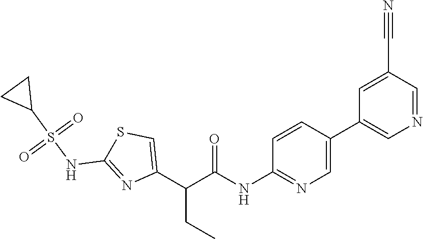 [SMILES: CCC(C(=O)Nc1ccc(-c2cncc(C#N)c2)cn1)c1csc(NS(=O)(=O)C2CC2)n1] |
| T181 | 2-(2- |
| | (cyclopropanesulfonamido)thiazol-4- |
| | yl)-N-(5-phenylpyridin-2- |
| | yl)butanamide |
| | 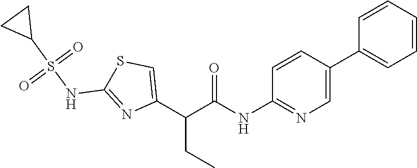 [SMILES: CCC(C(=O)Nc1ccc(-c2ccccc2)cn1)c1csc(NS(=O)(=O)C2CC2)n1] |
| T182 | 2-(2- |
| | (cyclopropanesulfonamido)thiazol-4- |
| | yl)-N-(5-(6-(2,2,2- |
| | trifluoroethoxy)pyrazin-2-yl)pyridin-2- |
| | yl)butanamide |
| | 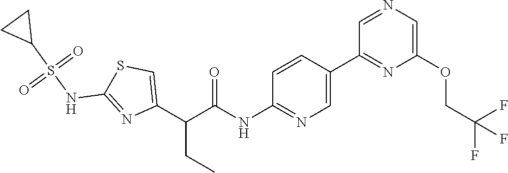 [SMILES: CCC(C(=O)Nc1ccc(-c2cncc(OCC(F)(F)F)n2)cn1)c1csc(NS(=O)(=O)C2CC2)n1] |
| T183 | 2-(2- |
| | (cyclopropanesulfonamido)thiazol-4- |
| | yl)-N-(5-(6-ethoxypyrazin-2-yl)-3- |
| | fluoropyridin-2-yl)butanamide |
| | 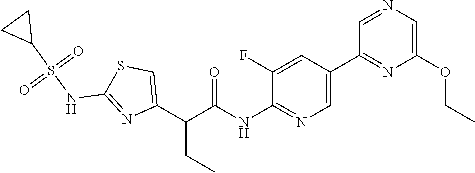 [SMILES: CCOc1cncc(-c2cnc(NC(=O)C(CC)c3csc(NS(=O)(=O)C4CC4)n3)c(F)c2)n1] |
| T184 | N-(5-(6-cyanopyrazin-2-yl)-3- |
| | fluoropyridin-2-yl)-2-(2- |
| | (cyclopropanesulfonamido)thiazol-4- |
| | yl)butanamide |
| | 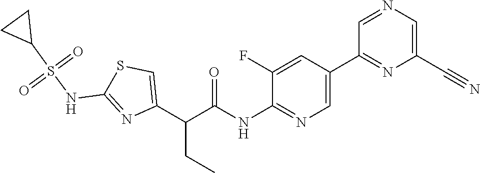 [SMILES: CCC(C(=O)Nc1ncc(-c2cncc(C#N)n2)cc1F)c1csc(NS(=O)(=O)C2CC2)n1] |
| T185 | 2-(2- |
| | (cyclopropanesulfonamido)thiazol-4- |
| | yl)-N-(5′-(2,2,2-trifluoroethoxy)-[3,3′- |
| | bipyridin]-6-yl)butanamide |
| | 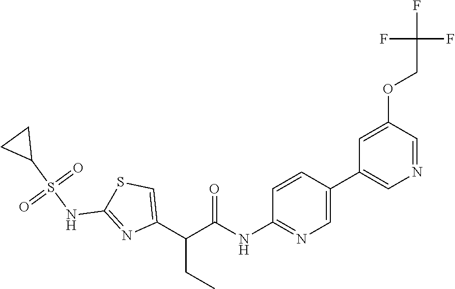 [SMILES: CCC(C(=O)Nc1ccc(-c2cncc(OCC(F)(F)F)c2)cn1)c1csc(NS(=O)(=O)C2CC2)n1] |
| T186 | 2-(2- |
| | (cyclopropanesulfonamido)thiazol-4- |
| | yl)-N-(5′-(difluoromethoxy)-[3,3′- |
| | bipyridin]-6-yl)butanamide |
| | 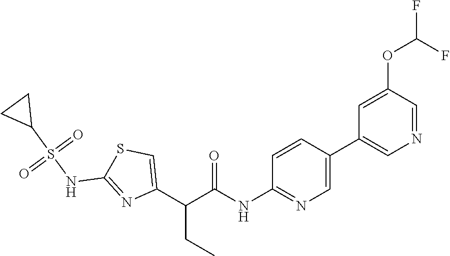 [SMILES: CCC(C(=O)Nc1ccc(-c2cncc(OC(F)F)c2)cn1)c1csc(NS(=O)(=O)C2CC2)n1] |
| T187 | N-(5-(6-chloropyrazin-2-yl)pyridin-2- |
| | yl)-2-(2- |
| | (cyclopropanesulfonamido)thiazol-4- |
| | yl)butanamide |
| | 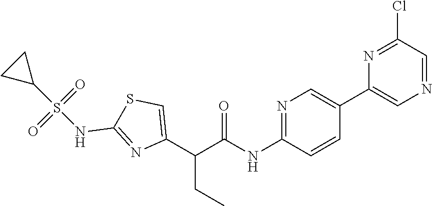 [SMILES: CCC(C(=O)Nc1ccc(-c2cncc(Cl)n2)cn1)c1csc(NS(=O)(=O)C2CC2)n1] |
| T188 | 2-(2- |
| | (cyclopropanesulfonamido)thiazol-4- |
| | yl)-N-(2,3-difluoro-4-(pyridin-3- |
| | yl)phenyl)acetamide |
| | 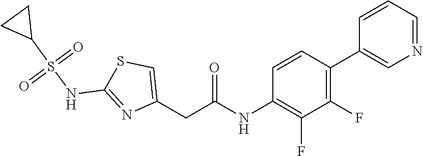 [SMILES: O=C(Cc1csc(NS(=O)(=O)C2CC2)n1)Nc1ccc(-c2cccnc2)c(F)c1F] |
| T189 | 2-(2- |
| | (cyclopropanesulfonamido)thiazol-4- |
| | yl)-N-(4-(6-ethoxypyrazin-2- |
| | yl)phenyl)butanamide |
| | 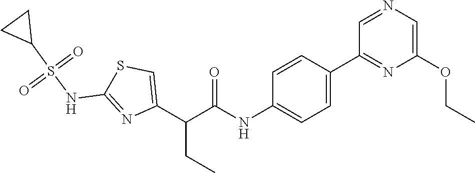 [SMILES: CCOc1cncc(-c2ccc(NC(=O)C(CC)c3csc(NS(=O)(=O)C4CC4)n3)cc2)n1] |
| T190 | 2-(2- |
| | (cyclopropanesulfonamido)thiazol-4- |
| | yl)-4-methoxy-N-(4-(6- |
| | methoxypyrazin-2- |
| | yl)phenyl)butanamide |
| | 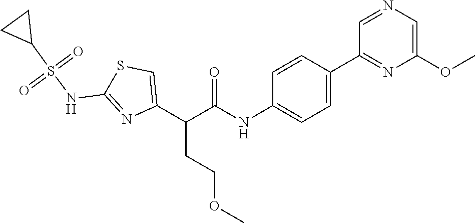 [SMILES: COCCC(C(=O)Nc1ccc(-c2cncc(OC)n2)cc1)c1csc(NS(=O)(=O)C2CC2)n1] |
| T191 | 2-(2- |
| | (cyclopropanesulfonamido)thiazol-4- |
| | yl)-N-(4-(5-fluoropyridin-3-yl)phenyl)- |
| | 4-methoxybutanamide |
| | 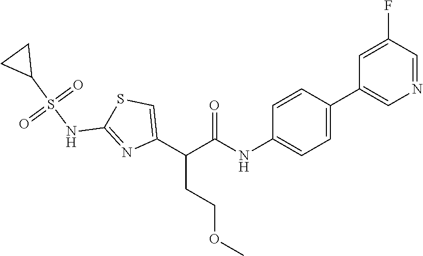 [SMILES: COCCC(C(=O)Nc1ccc(-c2cncc(F)c2)cc1)c1csc(NS(=O)(=O)C2CC2)n1] |
| T192 | 2-(2- |
| | (cyclopropanesulfonamido)thiazol-4- |
| | yl)-N-(4-(6-ethoxypyrazin-2- |
| | yl)phenyl)propanamide |
| | 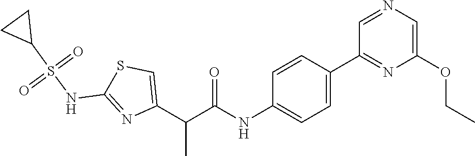 [SMILES: CCOc1cncc(-c2ccc(NC(=O)C(C)c3csc(NS(=O)(=O)C4CC4)n3)cc2)n1] |
| T193 | 2-(2- |
| | (cyclopropanesulfonamido)thiazol-4- |
| | yl)-N-(4-(5-(trifluoromethyl)pyridin-3- |
| | yl)phenyl)butanamide |
| | 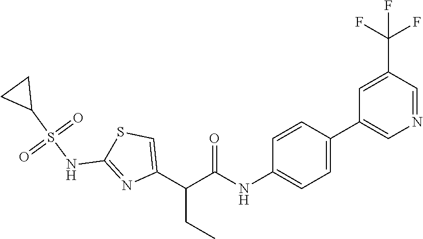 [SMILES: CCC(C(=O)Nc1ccc(-c2cncc(C(F)(F)F)c2)cc1)c1csc(NS(=O)(=O)C2CC2)n1] |
| T194 | N-(4-(5-cyanopyridin-3-yl)phenyl)-2- |
| | (2-(cyclopropanesulfonamido)thiazol- |
| | 4-yl)butanamide |
| | 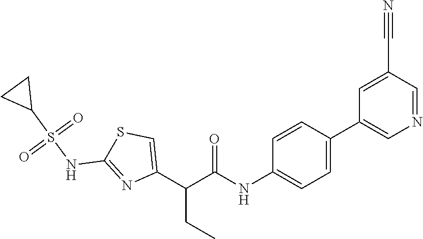 [SMILES: CCC(C(=O)Nc1ccc(-c2cncc(C#N)c2)cc1)c1csc(NS(=O)(=O)C2CC2)n1] |
| T195 | 2-(2- |
| | (cyclopropanesulfonamido)thiazol-4- |
| | yl)-N-(4-(6-(2,2,2- |
| | trifluoroethoxy)pyrazin-2- |
| | yl)phenyl)butanamide |
| | 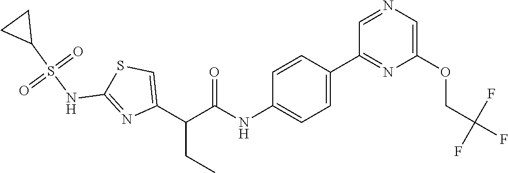 [SMILES: CCC(C(=O)Nc1ccc(-c2cncc(OCC(F)(F)F)n2)cc1)c1csc(NS(=O)(=O)C2CC2)n1] |
| T196 | 2-(2- |
| | (cyclopropanesulfonamido)thiazol-4- |
| | yl)-N-(2-fluoro-4-(pyrazin-2- |
| | yl)phenyl)butanamide |
| | 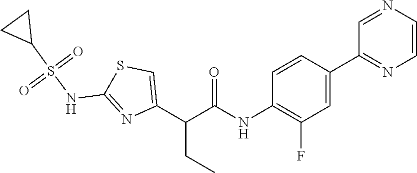 [SMILES: CCC(C(=O)Nc1ccc(-c2cnccn2)cc1F)c1csc(NS(=O)(=O)C2CC2)n1] |
| T197 | 2-(2-(cyclopropanesulfonamido)thiazol-4- |
| | yl)-N-(2-fluoro-4-(6- |
| | (trifluoromethyl)pyrazin-2- |
| | yl)phenyl)butanamide |
| | 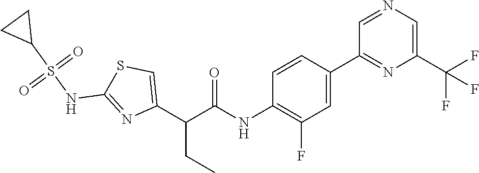 [SMILES: CCC(C(=O)Nc1ccc(-c2cncc(C(F)(F)F)n2)cc1F)c1csc(NS(=O)(=O)C2CC2)n1] |
| T198 | 2-(2-(cyclopropanesulfonamido)thiazol-4- |
| | yl)-N-(4-(6-ethoxypyrazin-2-yl)-2- |
| | fluorophenyl)butanamide |
| | 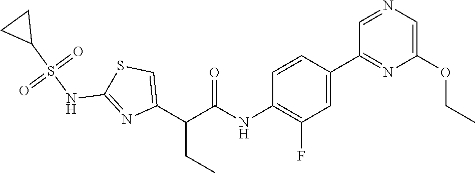 [SMILES: CCOc1cncc(-c2ccc(NC(=O)C(CC)c3csc(NS(=O)(=O)C4CC4)n3)c(F)c2)n1] |
| T199 | 2-(2-(cyclopropanesulfonamido)thiazol-4- |
| | yl)-N-(4-(5-ethoxypyridin-3-yl)-2- |
| | fluorophenyl)butanamide |
| | 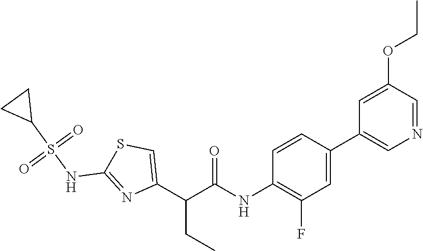 [SMILES: CCOc1cncc(-c2ccc(NC(=O)C(CC)c3csc(NS(=O)(=O)C4CC4)n3)c(F)c2)c1] |
| T200 | 2-(2-(cyclopropanesulfonamido)thiazol-4- |
| | yl)-N-(2-fluoro-4-(5-fluoropyridin-3- |
| | yl)phenyl)butanamide |
| | 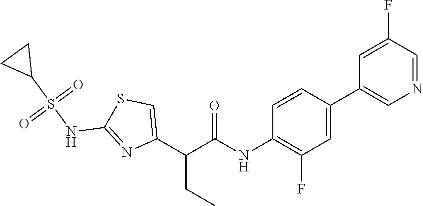 [SMILES: CCC(C(=O)Nc1ccc(-c2cncc(F)c2)cc1F)c1csc(NS(=O)(=O)C2CC2)n1] |
| T201 | 2-(2-(cyclopropanesulfonamido)thiazol-4- |
| | yl)-N-(2-fluoro-4-(pyridin-3- |
| | yl)phenyl)butanamide |
| | 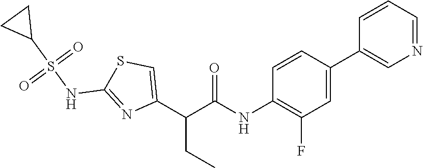 [SMILES: CCC(C(=O)Nc1ccc(-c2cccnc2)cc1F)c1csc(NS(=O)(=O)C2CC2)n1] |
| T202 | N-(4-(5-cyanopyridin-3-yl)-2-fluorophenyl)- |
| | 2-(2-(cyclopropanesulfonamido)thiazol-4- |
| | yl)butanamide |
| | 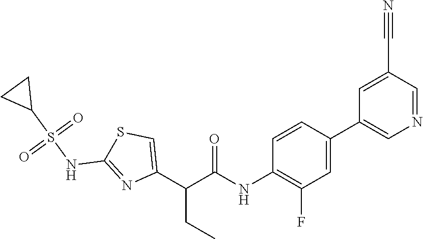 [SMILES: CCC(C(=O)Nc1ccc(-c2cncc(C#N)c2)cc1F)c1csc(NS(=O)(=O)C2CC2)n1] |
| T203 | N-(4-(5-chloropyridin-3-yl)-2-fluorophenyl)- |
| | 2-(2-(cyclopropanesulfonamido)thiazol-4- |
| | yl)butanamide |
| | 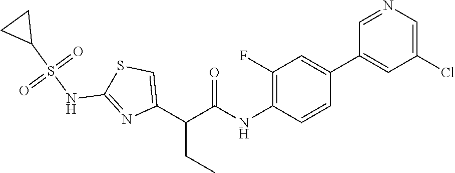 [SMILES: CCC(C(=O)Nc1ccc(-c2cncc(Cl)c2)cc1F)c1csc(NS(=O)(=O)C2CC2)n1] |
| T204 | Single enantiomer—stereochemistry |
| | unassigned |
| | 2-(2-(cyclopropanesulfonamido)thiazol-4- |
| | yl)-N-(4-(6-ethoxypyrazin-2- |
| | yl)phenyl)butanamide |
| | 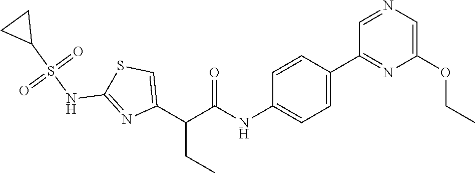 [SMILES: CCOc1cncc(-c2ccc(NC(=O)C(CC)c3csc(NS(=O)(=O)C4CC4)n3)cc2)n1] |
| T205 | Single enantiomer—stereochemistry |
| | unassigned 2-(2- |
| | (cyclopropanesulfonamido)thiazol-4-yl)-N- |
| | (4-(6-ethoxypyrazin-2- |
| | yl)phenyl)butanamide |
| | 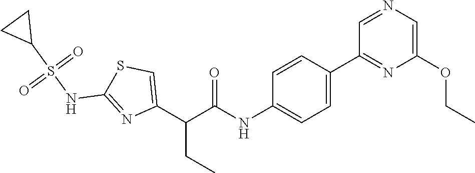 [SMILES: CCOc1cncc(-c2ccc(NC(=O)C(CC)c3csc(NS(=O)(=O)C4CC4)n3)cc2)n1] |
| T206 | N-(4-(1-(5-(6-ethoxypyrazin-2-yl)indolin-1- |
| | yl)-1-oxobutan-2-yl)thiazol-2- |
| | yl)cyclopropanesulfonamide |
| | 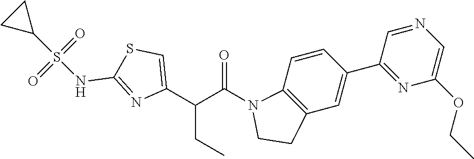 [SMILES: CCOc1cncc(-c2ccc3c(c2)CCN3C(=O)C(CC)c2csc(NS(=O)(=O)C3CC3)n2)n1] |
| T207 | 2-(2-(cyclopropanesulfonamido)thiazol-4- |
| | yl)-N-(2-fluoro-4-(6-(2,2,2- |
| | trifluoroethoxy)pyrazin-2- |
| | yl)phenyl)butanamide |
| | 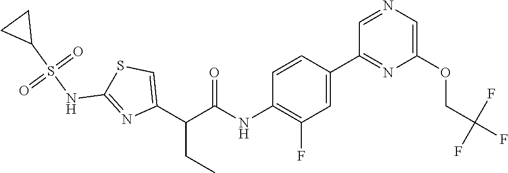 [SMILES: CCC(C(=O)Nc1ccc(-c2cncc(OCC(F)(F)F)n2)cc1F)c1csc(NS(=O)(=O)C2CC2)n1] |
| T208 | 2-(2-(cyclopropanesulfonamido)thiazol-4- |
| | yl)-N-(2-fluoro-4-(6-methoxypyrazin-2- |
| | yl)phenyl)butanamide |
| | 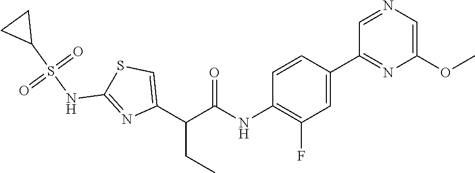 [SMILES: CCC(C(=O)Nc1ccc(-c2cncc(OC)n2)cc1F)c1csc(NS(=O)(=O)C2CC2)n1] |
| T209 | 2-(2-(cyclopropanesulfonamido)thiazol-4- |
| | yl)-N-(4-(5-(difluoromethoxy)pyridin-3-yl)- |
| | 2-fluorophenyl)butanamide |
| | 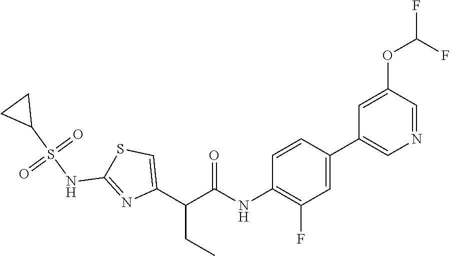 [SMILES: CCC(C(=O)Nc1ccc(-c2cncc(OC(F)F)c2)cc1F)c1csc(NS(=O)(=O)C2CC2)n1] |
| T210 | 2-(2-(cyclopropanesulfonamido)thiazol-4- |
| | yl)-N-(4-(5-(difluoromethoxy)pyridin-3- |
| | yl)phenyl)butanamide |
| | 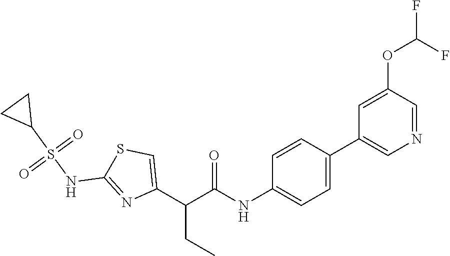 [SMILES: CCC(C(=O)Nc1ccc(-c2cncc(OC(F)F)c2)cc1)c1csc(NS(=O)(=O)C2CC2)n1] |
| T211 | 2-(2-(cyclopropanesulfonamido)thiazol-4- |
| | yl)-N-(4-(5-(2,2,2-trifluoroethoxy)pyridin-3- |
| | yl)phenyl)butanamide |
| | 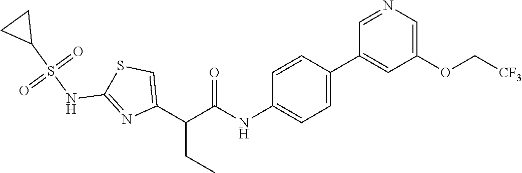 [SMILES: CCC(C(=O)Nc1ccc(-c2cncc(OCC(F)(F)F)c2)cc1)c1csc(NS(=O)(=O)C2CC2)n1] |
| T212 | 2-(2-(cyclopropanesulfonamido)thiazol-4- |
| | yl)-N-(2-fluoro-4-(5-(2,2,2- |
| | trifluoroethoxy)pyridin-3- |
| | yl)phenyl)butanamide |
| | 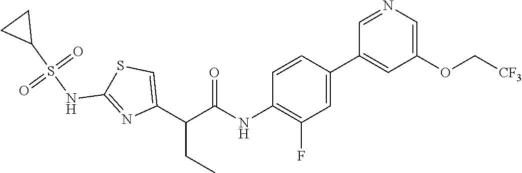 [SMILES: CCC(C(=O)Nc1ccc(-c2cncc(OCC(F)(F)F)c2)cc1F)c1csc(NS(=O)(=O)C2CC2)n1] |
| T213 | 2-(cyclopropanesulfonamido)-N-(4- |
| | (pyridin-3-yl)phenyl)-5,6-dihydro-4H- |
| | cyclopenta[d]thiazole-4-carboxamide |
| | 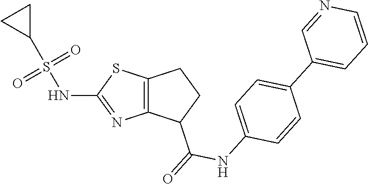 [SMILES: O=C(Nc1ccc(-c2cccnc2)cc1)C1CCc2sc(NS(=O)(=O)C3CC3)nc21] |
| T214 | 2-(2-(cyclopropanesulfonamido)thiazol-4- |
| | yl)-2-methoxy-N-(5-(6- |
| | (trifluoromethyl)pyrazin-2-yl)pyridin-2- |
| | yl)acetamide |
| | 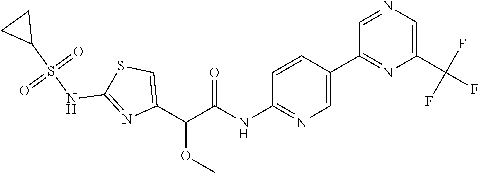 [SMILES: COC(C(=O)Nc1ccc(-c2cncc(C(F)(F)F)n2)cn1)c1csc(NS(=O)(=O)C2CC2)n1] |
| T215 | 2-(2-(cyclopropanesulfonamido)thiazol-4- |
| | yl)-N-(2-methoxy-4-(pyridin-3- |
| | yl)phenyl)acetamide |
| | 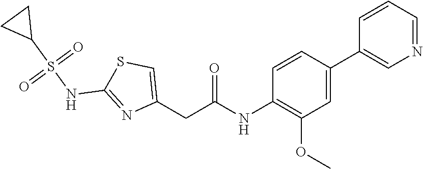 [SMILES: COc1cc(-c2cccnc2)ccc1NC(=O)Cc1csc(NS(=O)(=O)C2CC2)n1] |
| T216 | 2-(2-(cyclopropanesulfonamido)thiazol-4- |
| | yl)-N-(2-fluoro-4-(pyridin-3- |
| | yl)phenyl)acetamide |
| | 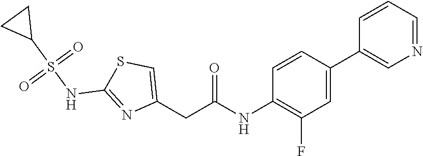 [SMILES: O=C(Cc1csc(NS(=O)(=O)C2CC2)n1)Nc1ccc(-c2cccnc2)cc1F] |
| T217 | N-(4-(5-cyanopyridin-3-yl)phenyl)-2- |
| | (cyclopropanesulfonamido)-5,6-dihydro- |
| | 4H-cyclopenta[d]thiazole-4-carboxamide |
| | 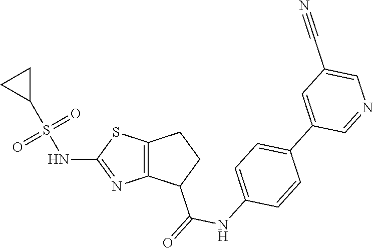 [SMILES: N#Cc1cncc(-c2ccc(NC(=O)C3CCc4sc(NS(=O)(=O)C5CC5)nc43)cc2)c1] |
| T218 | 2-(cyclopropanesulfonamido)-N-(4-(5- |
| | fluoropyridin-3-yl)phenyl)-5,6-dihydro-4H- |
| | cyclopenta[d]thiazole-4-carboxamide |
| | 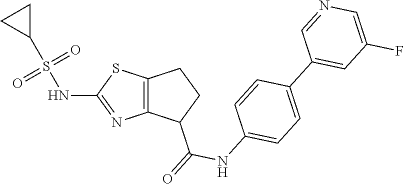 [SMILES: O=C(Nc1ccc(-c2cncc(F)c2)cc1)C1CCc2sc(NS(=O)(=O)C3CC3)nc21] |
| T219 | 2-(2-(cyclopropanesulfonamido)thiazol-4- |
| | yl)-2-methoxy-N-(4-(pyridin-3- |
| | yl)phenyl)acetamide |
| | 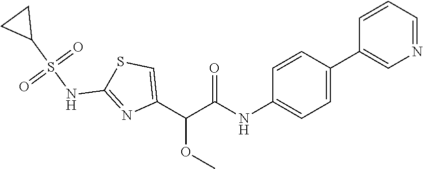 [SMILES: COC(C(=O)Nc1ccc(-c2cccnc2)cc1)c1csc(NS(=O)(=O)C2CC2)n1] |
| T220 | 2-(2-(cyclopropanesulfonamido)thiazol-4- |
| | yl)-N-(2-fluoro-4-(pyridin-3-yl)phenyl)-2- |
| | methoxyacetamide |
| | 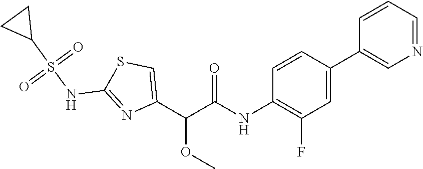 [SMILES: COC(C(=O)Nc1ccc(-c2cccnc2)cc1F)c1csc(NS(=O)(=O)C2CC2)n1] |
| T221 | N-(2-chloro-4-(pyridin-3-yl)phenyl)-2-(2- |
| | (cyclopropanesulfonamido)thiazol-4- |
| | yl)butanamide |
| | 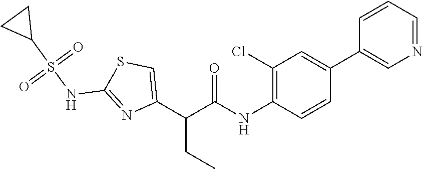 [SMILES: CCC(C(=O)Nc1ccc(-c2cccnc2)cc1Cl)c1csc(NS(=O)(=O)C2CC2)n1] |
| T222 | Single enantiomer—stereochemistry |
| | unassigned |
| | 2-(2-(cyclopropanesulfonamido)thiazol-4- |
| | yl)-N-(4-(6-(2,2,2-trifluoroethoxy)pyrazin-2- |
| | yl)phenyl)butanamide |
| | 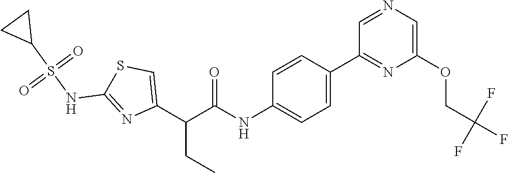 [SMILES: CCC(C(=O)Nc1ccc(-c2cncc(OCC(F)(F)F)n2)cc1)c1csc(NS(=O)(=O)C2CC2)n1] |
| T223 | Single enantiomer—stereochemistry |
| | unassigned |
| | 2-(2-(cyclopropanesulfonamido)thiazol-4- |
| | yl)-N-(4-(6-(2,2,2-trifluoroethoxy)pyrazin-2- |
| | yl)phenyl)butanamide |
| | 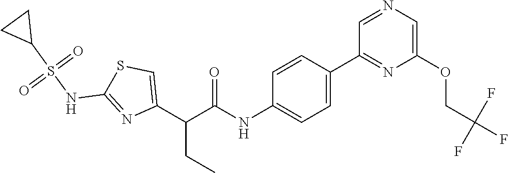 [SMILES: CCC(C(=O)Nc1ccc(-c2cncc(OCC(F)(F)F)n2)cc1)c1csc(NS(=O)(=O)C2CC2)n1] |
| T224 | 2-(2-(cyclopropanesulfonamido)thiazol-4- |
| | yl)-N-(5′-methoxy-[3,3′-bipyridin]-6-yl)-2- |
| | methylpropanamide |
| | 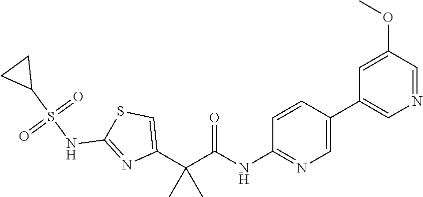 [SMILES: COc1cncc(-c2ccc(NC(=O)C(C)(C)c3csc(NS(=O)(=O)C4CC4)n3)nc2)c1] |
| T225 | N-(5′-chloro-[3,3′-bipyridin]-6-yl)-2-(2- |
| | (cyclopropanesulfonamido)thiazol-4-yl)-2- |
| | methylpropanamide |
| | 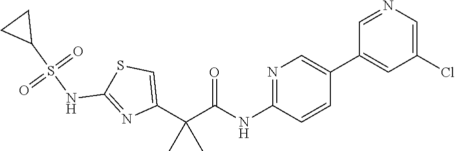 [SMILES: CC(C)(C(=O)Nc1ccc(-c2cncc(Cl)c2)cn1)c1csc(NS(=O)(=O)C2CC2)n1] |
| T226 | N-(5′-cyano-[3,3′-bipyridin]-6-yl)-2-(2- |
| | (cyclopropanesulfonamido)thiazol-4-yl)-2- |
| | methylpropanamide |
| | 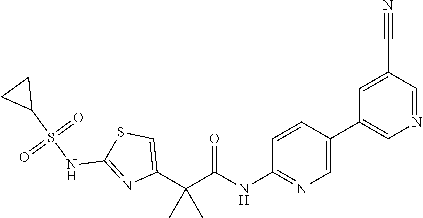 [SMILES: CC(C)(C(=O)Nc1ccc(-c2cncc(C#N)c2)cn1)c1csc(NS(=O)(=O)C2CC2)n1] |
| T227 | 2-(2-(cyclopropanesulfonamido)thiazol-4- |
| | yl)-N-(5-fluoro-[3,3′-bipyridin]-6-yl)-2- |
| | methylpropanamide |
| | 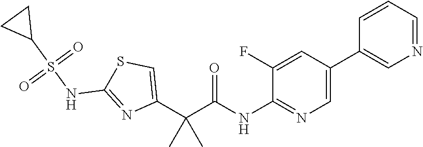 [SMILES: CC(C)(C(=O)Nc1ncc(-c2cccnc2)cc1F)c1csc(NS(=O)(=O)C2CC2)n1] |
| T228 | N-(5′-cyano-5-fluoro-[3,3′-bipyridin]-6-yl)-2- |
| | (2-(cyclopropanesulfonamido)thiazol-4-yl)- |
| | 2-methylpropanamide |
| | 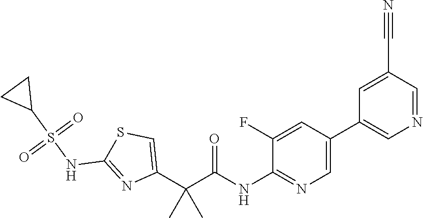 [SMILES: CC(C)(C(=O)Nc1ncc(-c2cncc(C#N)c2)cc1F)c1csc(NS(=O)(=O)C2CC2)n1] |
| T229 | N-(5′-chloro-5-fluoro-[3,3′-bipyridin]-6-yl)-2- |
| | (2-(cyclopropanesulfonamido)thiazol-4-yl)- |
| | 2-methylpropanamide |
| | 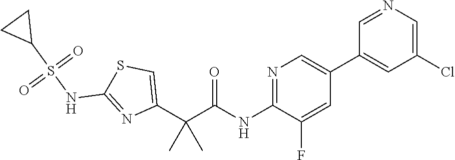 [SMILES: CC(C)(C(=O)Nc1ncc(-c2cncc(Cl)c2)cc1F)c1csc(NS(=O)(=O)C2CC2)n1] |
| T230 | 2-(2-(cyclopropanesulfonamido)thiazol-4- |
| | yl)-N-(5,5′-difluoro-[3,3′-bipyridin]-6-yl)-2- |
| | methylpropanamide |
| | 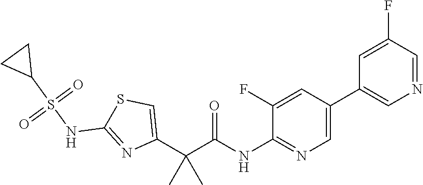 [SMILES: CC(C)(C(=O)Nc1ncc(-c2cncc(F)c2)cc1F)c1csc(NS(=O)(=O)C2CC2)n1] |
| T231 | N-(5-(3-chloro-5-methylphenyl)pyridin-2- |
| | yl)-2-(2-(cyclopropanesulfonamido)thiazol- |
| | 4-yl)-2-methylpropanamide |
| | 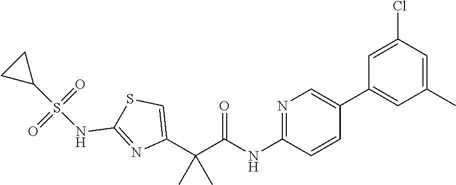 [SMILES: Cc1cc(Cl)cc(-c2ccc(NC(=O)C(C)(C)c3csc(NS(=O)(=O)C4CC4)n3)nc2)c1] |
| T232 | 2-(2-(cyclopropanesulfonamido)thiazol-4- |
| | yl)-N-(5-(3-methoxyphenyl)pyridin-2-yl)-2- |
| | methylpropanamide |
| | 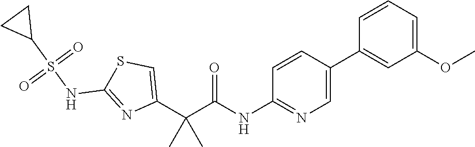 [SMILES: COc1cccc(-c2ccc(NC(=O)C(C)(C)c3csc(NS(=O)(=O)C4CC4)n3)nc2)c1] |
| T233 | 2-(2-(cyclopropanesulfonamido)thiazol-4- |
| | yl)-N-(5-(3-fluoro-5-methoxyphenyl)pyridin- |
| | 2-yl)-2-methylpropanamide |
| | 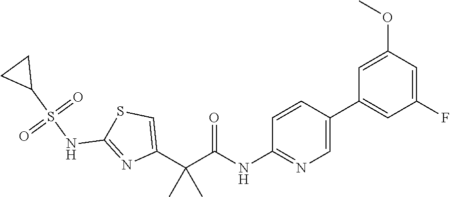 [SMILES: COc1cc(F)cc(-c2ccc(NC(=O)C(C)(C)c3csc(NS(=O)(=O)C4CC4)n3)nc2)c1] |
| T234 | 2-(2-(cyclopropanesulfonamido)thiazol-4- |
| | yl)-N-(5-(3,5-dimethoxyphenyl)pyridin-2- |
| | yl)-2-methylpropanamide |
| | 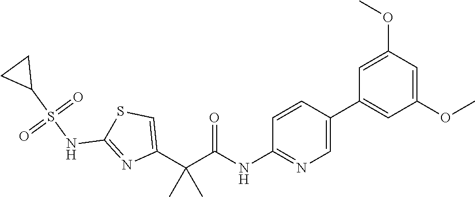 [SMILES: COc1cc(OC)cc(-c2ccc(NC(=O)C(C)(C)c3csc(NS(=O)(=O)C4CC4)n3)nc2)c1] |
| T235 | 2-(2-(cyclopropanesulfonamido)thiazol-4- |
| | yl)-2-methyl-N-(5-(3- |
| | (trifluoromethyl)phenyl)pyridin-2- |
| | yl)propanamide |
| | 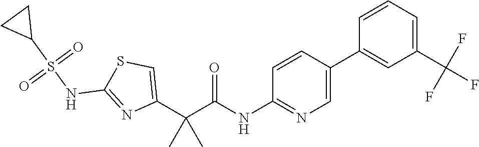 [SMILES: CC(C)(C(=O)Nc1ccc(-c2cccc(C(F)(F)F)c2)cn1)c1csc(NS(=O)(=O)C2CC2)n1] |
| T236 | 2-(2-(cyclopropanesulfonamido)thiazol-4- |
| | yl)-2-methyl-N-(5-(3- |
| | (trifluoromethoxy)phenyl)pyridin-2- |
| | yl)propanamide |
| | 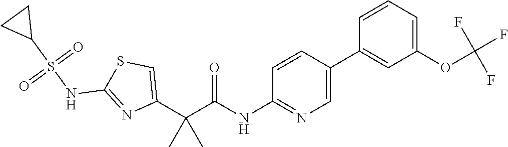 [SMILES: CC(C)(C(=O)Nc1ccc(-c2cccc(OC(F)(F)F)c2)cn1)c1csc(NS(=O)(=O)C2CC2)n1] |
| T237 | 2-(2-(cyclopropanesulfonamido)thiazol-4- |
| | yl)-N-(5-(3-(2-hydroxypropan-2- |
| | yl)phenyl)pyridin-2-yl)-2- |
| | methylpropanamide |
| | 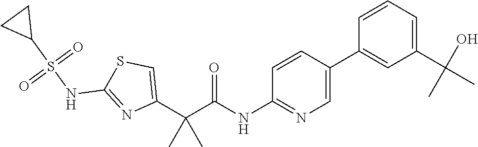 [SMILES: CC(C)(O)c1cccc(-c2ccc(NC(=O)C(C)(C)c3csc(NS(=O)(=O)C4CC4)n3)nc2)c1] |
| T238 | 2-(2-(cyclopropanesulfonamido)thiazol-4- |
| | yl)-2-methyl-N-(5-(3- |
| | morpholinophenyl)pyridin-2- |
| | yl)propanamide |
| | 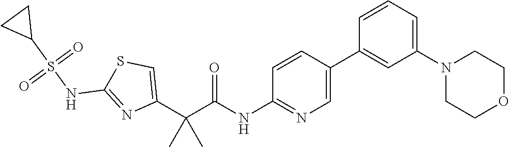 [SMILES: CC(C)(C(=O)Nc1ccc(-c2cccc(N3CCOCC3)c2)cn1)c1csc(NS(=O)(=O)C2CC2)n1] |
| T239 | 2-(2-(cyclopropanesulfonamido)thiazol-4- |
| | yl)-2-methyl-N-(6-phenylpyridin-3- |
| | yl)propanamide |
| | 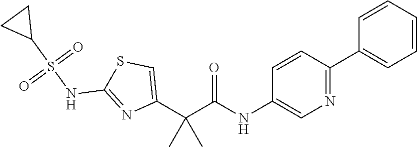 [SMILES: CC(C)(C(=O)Nc1ccc(-c2ccccc2)nc1)c1csc(NS(=O)(=O)C2CC2)n1] |
| T240 | 2-(2-(cyclopropanesulfonamido)thiazol-4- |
| | yl)-N-(4-(2-fluoropyridin-3-yl)phenyl)-2- |
| | methylpropanamide |
| | 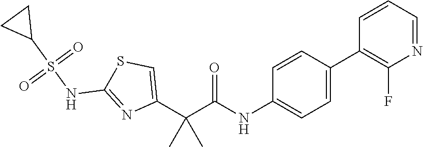 [SMILES: CC(C)(C(=O)Nc1ccc(-c2cccnc2F)cc1)c1csc(NS(=O)(=O)C2CC2)n1] |
| T241 | 2-(2-(cyclopropanesulfonamido)thiazol-4- |
| | yl)-N-(4-(5-(hydroxymethyl)pyridin-3- |
| | yl)phenyl)-2-methylpropanamide |
| | 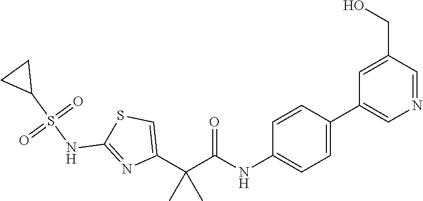 [SMILES: CC(C)(C(=O)Nc1ccc(-c2cncc(CO)c2)cc1)c1csc(NS(=O)(=O)C2CC2)n1] |
| T242 | 2-(2-(cyclopropanesulfonamido)thiazol-4- |
| | yl)-N-(4-(2-methoxypyrimidin-5- |
| | yl)phenyl)acetamide |
| | 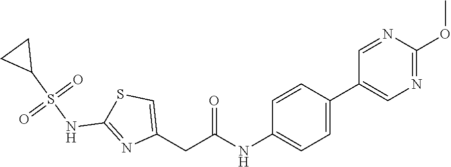 [SMILES: COc1ncc(-c2ccc(NC(=O)Cc3csc(NS(=O)(=O)C4CC4)n3)cc2)cn1] |
| T243 | N-(4′-(tert-butyl)-[1,1′-biphenyl]-4-yl)-2-(2- |
| | (cyclopropanesulfonamido)thiazol-4- |
| | yl)acetamide |
| | 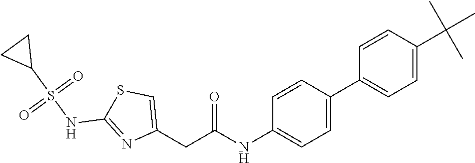 [SMILES: CC(C)(C)c1ccc(-c2ccc(NC(=O)Cc3csc(NS(=O)(=O)C4CC4)n3)cc2)cc1] |
| T244 | 2-(2-(cyclopropanesulfonamido)thiazol-4- |
| | yl)-N-(4-(pyridin-3-yl)phenyl)acetamide |
| | 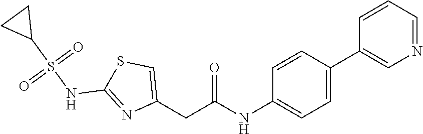 [SMILES: O=C(Cc1csc(NS(=O)(=O)C2CC2)n1)Nc1ccc(-c2cccnc2)cc1] |
| T245 | 2-(2-(cyclopropanesulfonamido)thiazol-4- |
| | yl)-N-(4-(pyridin-4-yl)phenyl)acetamide |
| | 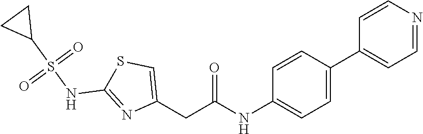 [SMILES: O=C(Cc1csc(NS(=O)(=O)C2CC2)n1)Nc1ccc(-c2ccncc2)cc1] |
| T246 | 2-(2-(cyclopropanesulfonamido)thiazol-4- |
| | yl)-N-(2′-methoxy-[1,1′-biphenyl]-4- |
| | yl)acetamide |
| | 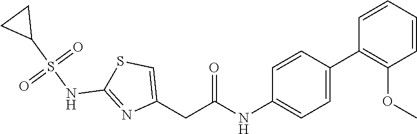 [SMILES: COc1ccccc1-c1ccc(NC(=O)Cc2csc(NS(=O)(=O)C3CC3)n2)cc1] |
| T247 | 2-(2-(cyclopropanesulfonamido)thiazol-4- |
| | yl)-N-(4-(pyrimidin-5-yl)phenyl)acetamide |
| | 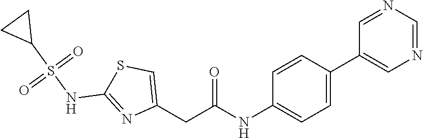 [SMILES: O=C(Cc1csc(NS(=O)(=O)C2CC2)n1)Nc1ccc(-c2cncnc2)cc1] |
| T248 | 2-(2-(cyclopropanesulfonamido)thiazol-4- |
| | yl)-2-methyl-N-(4-(2- |
| | (trifluoromethyl)pyridin-3- |
| | yl)phenyl)propanamide |
| | 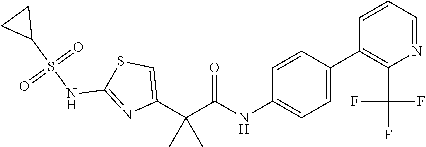 [SMILES: CC(C)(C(=O)Nc1ccc(-c2cccnc2C(F)(F)F)cc1)c1csc(NS(=O)(=O)C2CC2)n1] |
| T249 | 2-(2-(cyclopropanesulfonamido)thiazol-4- |
| | yl)-2-methyl-N-(5′-methyl-[3,3′-bipyridin]-6- |
| | yl)propanamide |
| | 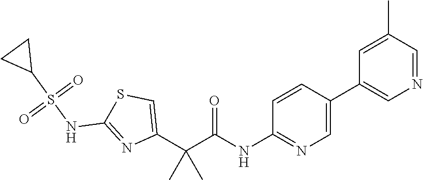 [SMILES: Cc1cncc(-c2ccc(NC(=O)C(C)(C)c3csc(NS(=O)(=O)C4CC4)n3)nc2)c1] |
| T250 | 2-(2-(cyclopropanesulfonamido)thiazol-4- |
| | yl)-N-(4-(2-methoxy-4-methylpyridin-3- |
| | yl)phenyl)-2-methylpropanamide |
| | 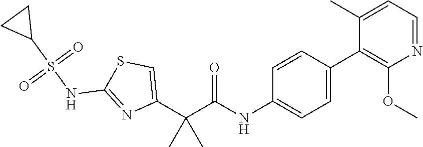 [SMILES: COc1nccc(C)c1-c1ccc(NC(=O)C(C)(C)c2csc(NS(=O)(=O)C3CC3)n2)cc1] |
| T251 | 2-(2-(cyclopropanesulfonamido)thiazol-4- |
| | yl)-N-(4-(6-methoxy-5-methylpyridin-3- |
| | yl)phenyl)-2-methylpropanamide |
| | 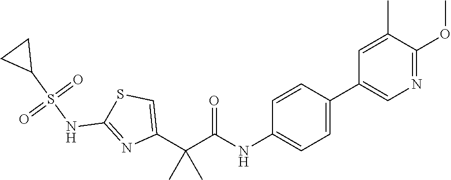 [SMILES: COc1ncc(-c2ccc(NC(=O)C(C)(C)c3csc(NS(=O)(=O)C4CC4)n3)cc2)cc1C] |
| T252 | N-(4-(5-chloropyridin-3-yl)phenyl)-2-(2- |
| | (cyclopropanesulfonamido)thiazol-4-yl)-2- |
| | methylpropanamide |
| | 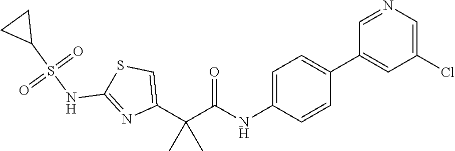 [SMILES: CC(C)(C(=O)Nc1ccc(-c2cncc(Cl)c2)cc1)c1csc(NS(=O)(=O)C2CC2)n1] |
| T253 | 2-(2-(cyclopropanesulfonamido)thiazol-4- |
| | yl)-N-(4-(5-fluoropyridin-3-yl)phenyl)-2- |
| | methylpropanamide |
| | 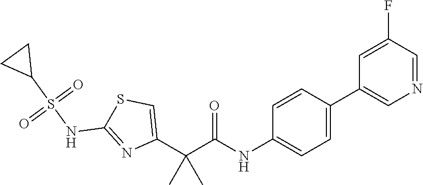 [SMILES: CC(C)(C(=O)Nc1ccc(-c2cncc(F)c2)cc1)c1csc(NS(=O)(=O)C2CC2)n1] |
| T254 | 2-(2-(cyclopropanesulfonamido)thiazol-4- |
| | yl)-2-methyl-N-(4-(4-methylpyridin-3- |
| | yl)phenyl)propanamide |
| | 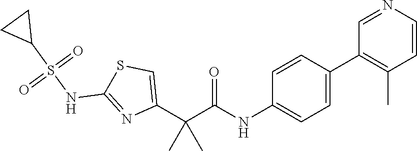 [SMILES: Cc1ccncc1-c1ccc(NC(=O)C(C)(C)c2csc(NS(=O)(=O)C3CC3)n2)cc1] |
| T255 | 2-(2-(cyclopropanesulfonamido)thiazol-4- |
| | yl)-2-methyl-N-(4-(4- |
| | (trifluoromethyl)pyridin-3- |
| | yl)phenyl)propanamide |
| | 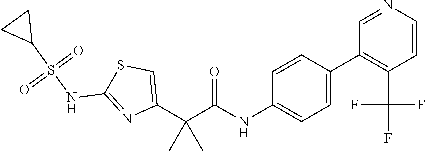 [SMILES: CC(C)(C(=O)Nc1ccc(-c2cnccc2C(F)(F)F)cc1)c1csc(NS(=O)(=O)C2CC2)n1] |
| T256 | N-(4-(5-chloropyridin-3-yl)-2- |
| | methoxyphenyl)-2-(2- |
| | (cyclopropanesulfonamido)thiazol-4-yl)-2- |
| | methylpropanamide |
| | 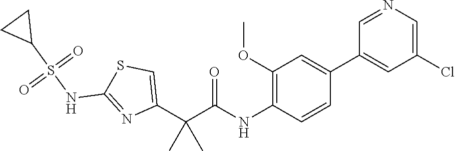 [SMILES: COc1cc(-c2cncc(Cl)c2)ccc1NC(=O)C(C)(C)c1csc(NS(=O)(=O)C2CC2)n1] |
| T257 | 2-(2-(cyclopropanesulfonamido)thiazol-4- |
| | yl)-N-(4-(5-(dimethylamino)pyridin-3- |
| | yl)phenyl)-2-methylpropanamide |
| | 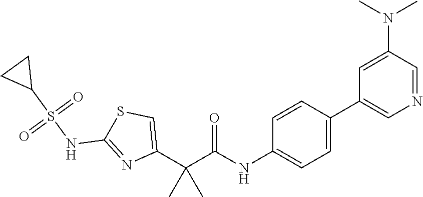 [SMILES: CN(C)c1cncc(-c2ccc(NC(=O)C(C)(C)c3csc(NS(=O)(=O)C4CC4)n3)cc2)c1] |
| T258 | 2-(2-(cyclopropanesulfonamido)thiazol-4- |
| | yl)-N-(2-methoxy-4-(5-methylpyridin-3- |
| | yl)phenyl)-2-methylpropanamide |
| | 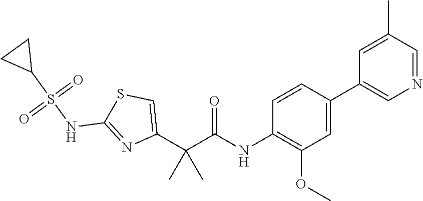 [SMILES: COc1cc(-c2cncc(C)c2)ccc1NC(=O)C(C)(C)c1csc(NS(=O)(=O)C2CC2)n1] |
| T259 | 2-(2-(cyclopropanesulfonamido)thiazol-4- |
| | yl)-N-(2-methoxy-4-(5- |
| | (trifluoromethyl)pyridin-3-yl)phenyl)-2- |
| | methylpropanamide |
| | 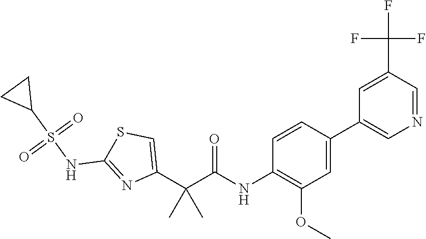 [SMILES: COc1cc(-c2cncc(C(F)(F)F)c2)ccc1NC(=O)C(C)(C)c1csc(NS(=O)(=O)C2CC2)n1] |
| T260 | 2-(2-(cyclopropanesulfonamido)thiazol-4- |
| | yl)-N-(4-(6-methoxypyridin-3- |
| | yl)phenyl)acetamide |
| | 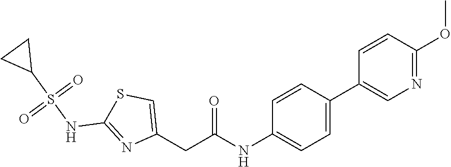 [SMILES: COc1ccc(-c2ccc(NC(=O)Cc3csc(NS(=O)(=O)C4CC4)n3)cc2)cn1] |
| T261 | 2-(2-(cyclopropanesulfonamido)thiazol-4- |
| | yl)-N-(5′-fluoro-[3,3′-bipyridin]-6-yl)-2- |
| | methylpropanamide |
| | 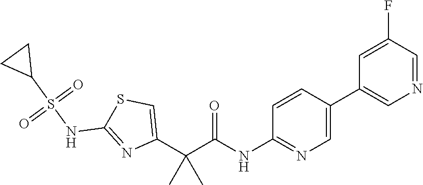 [SMILES: CC(C)(C(=O)Nc1ccc(-c2cncc(F)c2)cn1)c1csc(NS(=O)(=O)C2CC2)n1] |
| T262 | N-(5-(6-chloropyrazin-2-yl)pyridin-2-yl)-2- |
| | (2-(cyclopropanesulfonamido)thiazol-4-yl)- |
| | 2-methylpropanamide |
| | 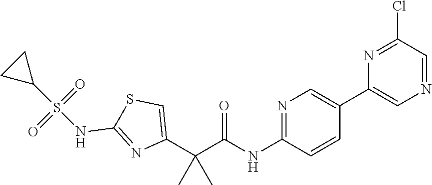 [SMILES: CC(C)(C(=O)Nc1ccc(-c2cncc(Cl)n2)cn1)c1csc(NS(=O)(=O)C2CC2)n1] |
| T263 | N-(5-(6-cyanopyrazin-2-yl)pyridin-2-yl)-2- |
| | (2-(cyclopropanesulfonamido)thiazol-4-yl)- |
| | 2-methylpropanamide |
| | 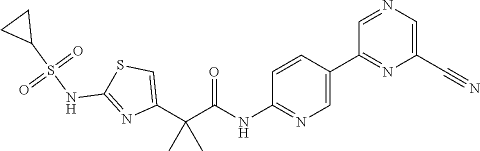 [SMILES: CC(C)(C(=O)Nc1ccc(-c2cncc(C#N)n2)cn1)c1csc(NS(=O)(=O)C2CC2)n1] |
| T264 | 2-(2-(cyclopropanesulfonamido)thiazol-4- |
| | yl)-2-methyl-N-(5-(pyrimidin-5-yl)pyridin-2- |
| | yl)propanamide |
| | 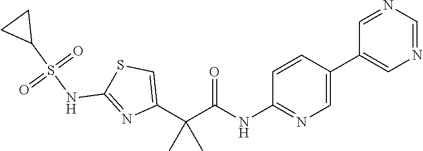 [SMILES: CC(C)(C(=O)Nc1ccc(-c2cncnc2)cn1)c1csc(NS(=O)(=O)C2CC2)n1] |
| T265 | 2-(2-(cyclopropanesulfonamido)thiazol-4- |
| | yl)-N-(4-(6-methoxypyrazin-2-yl)phenyl)-2- |
| | methylpropanamide |
| | 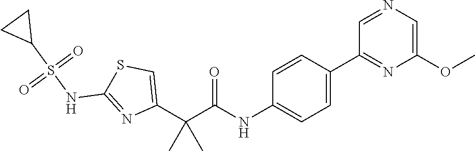 [SMILES: COc1cncc(-c2ccc(NC(=O)C(C)(C)c3csc(NS(=O)(=O)C4CC4)n3)cc2)n1] |
| T266 | 2-(2-(cyclopropanesulfonamido)thiazol-4- |
| | yl)-2-methyl-N-(4-(6-methylpyrazin-2- |
| | yl)phenyl)propanamide |
| | 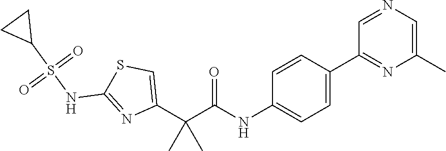 [SMILES: Cc1cncc(-c2ccc(NC(=O)C(C)(C)c3csc(NS(=O)(=O)C4CC4)n3)cc2)n1] |
| T267 | 2-(2-(cyclopropanesulfonamido)thiazol-4- |
| | yl)-2-methyl-N-(4-(6- |
| | (trifluoromethyl)pyrazin-2- |
| | yl)phenyl)propanamide |
| | 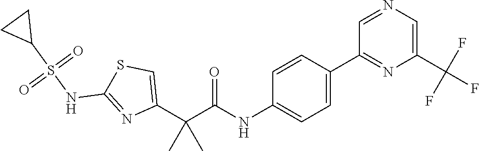 [SMILES: CC(C)(C(=O)Nc1ccc(-c2cncc(C(F)(F)F)n2)cc1)c1csc(NS(=O)(=O)C2CC2)n1] |
| T268 | N-(4-(6-chloropyridin-2-yl)phenyl)-2-(2- |
| | (cyclopropanesulfonamido)thiazol-4-yl)-2- |
| | methylpropanamide |
| | 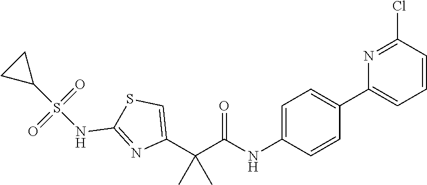 [SMILES: CC(C)(C(=O)Nc1ccc(-c2cccc(Cl)n2)cc1)c1csc(NS(=O)(=O)C2CC2)n1] |
| T269 | 2-(2-(cyclopropanesulfonamido)thiazol-4- |
| | yl)-N-(4-(6-methoxypyridin-2-yl)phenyl)-2- |
| | methylpropanamide |
| | 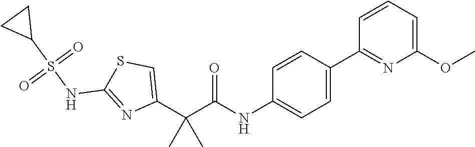 [SMILES: COc1cccc(-c2ccc(NC(=O)C(C)(C)c3csc(NS(=O)(=O)C4CC4)n3)cc2)n1] |
| T270 | 2-(2-(cyclopropanesulfonamido)thiazol-4- |
| | yl)-2-methyl-N-(4-(6- |
| | (trifluoromethyl)pyridin-2- |
| | yl)phenyl)propanamide |
| | 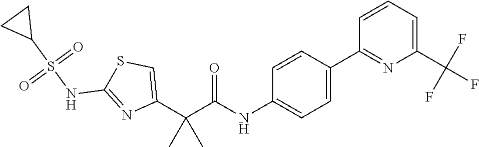 [SMILES: CC(C)(C(=O)Nc1ccc(-c2cccc(C(F)(F)F)n2)cc1)c1csc(NS(=O)(=O)C2CC2)n1] |
| T271 | 2-(2-(cyclopropanesulfonamido)thiazol-4- |
| | yl)-N-(4-(4-methoxypyridin-2-yl)phenyl)-2- |
| | methylpropanamide |
| | 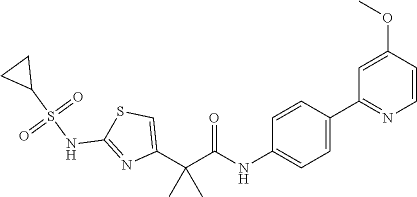 [SMILES: COc1ccnc(-c2ccc(NC(=O)C(C)(C)c3csc(NS(=O)(=O)C4CC4)n3)cc2)c1] |
| T272 | 2-(2-(cyclopropanesulfonamido)thiazol-4- |
| | yl)-N-(4-(6-isopropoxypyrazin-2-yl)phenyl)- |
| | 2-methylpropanamide |
| | 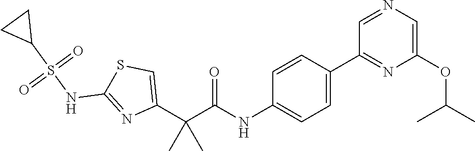 [SMILES: CC(C)Oc1cncc(-c2ccc(NC(=O)C(C)(C)c3csc(NS(=O)(=O)C4CC4)n3)cc2)n1] |
| T273 | 2-(2-(cyclopropanesulfonamido)thiazol-4- |
| | yl)-N-(4-(6-cyclopropoxypyrazin-2- |
| | yl)phenyl)-2-methylpropanamide |
| | 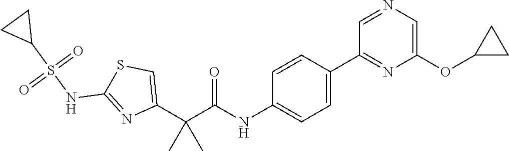 [SMILES: CC(C)(C(=O)Nc1ccc(-c2cncc(OC3CC3)n2)cc1)c1csc(NS(=O)(=O)C2CC2)n1] |
| T274 | 2-(2-(cyclopropanesulfonamido)thiazol-4- |
| | yl)-N-(2-fluoro-4-(pyrazin-2-yl)phenyl)-2- |
| | methylpropanamide |
| | 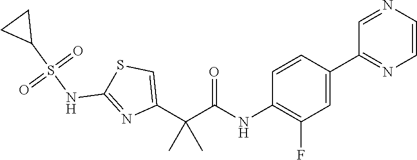 [SMILES: CC(C)(C(=O)Nc1ccc(-c2cnccn2)cc1F)c1csc(NS(=O)(=O)C2CC2)n1] |
| T275 | 2-(2-(cyclopropanesulfonamido)thiazol-4- |
| | yl)-N-(2-fluoro-4-(6-methoxypyrazin-2- |
| | yl)phenyl)-2-methylpropanamide |
| | 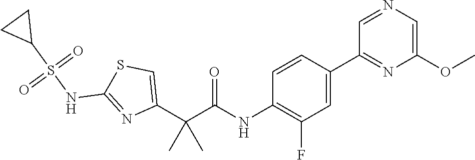 [SMILES: COc1cncc(-c2ccc(NC(=O)C(C)(C)c3csc(NS(=O)(=O)C4CC4)n3)c(F)c2)n1] |
| T276 | N-(4-(6-chloro-3-methylpyrazin-2- |
| | yl)phenyl)-2-(2- |
| | (cyclopropanesulfonamido)thiazol-4-yl)-2- |
| | methylpropanamide |
| | 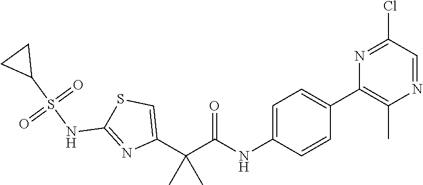 [SMILES: Cc1ncc(Cl)nc1-c1ccc(NC(=O)C(C)(C)c2csc(NS(=O)(=O)C3CC3)n2)cc1] |
| T277 | N-(4-(6-chloro-5-methylpyrazin-2- |
| | yl)phenyl)-2-(2- |
| | (cyclopropanesulfonamido)thiazol-4-yl)-2- |
| | methylpropanamide |
| | 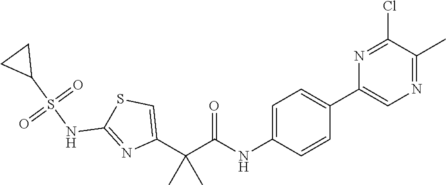 [SMILES: Cc1ncc(-c2ccc(NC(=O)C(C)(C)c3csc(NS(=O)(=O)C4CC4)n3)cc2)nc1Cl] |
| T278 | 2-(2-(cyclopropanesulfonamido)thiazol-4- |
| | yl)-2-methyl-N-(4-(6-(pyrrolidin-1- |
| | yl)pyrazin-2-yl)phenyl)propanamide |
| | 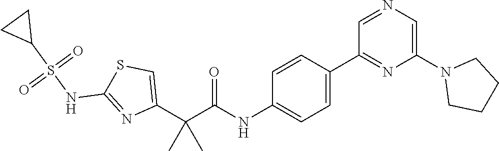 [SMILES: CC(C)(C(=O)Nc1ccc(-c2cncc(N3CCCC3)n2)cc1)c1csc(NS(=O)(=O)C2CC2)n1] |
| T279 | 2-(2-(cyclopropanesulfonamido)thiazol-4- |
| | yl)-N-(4-(6-(2- |
| | (dimethylamino)ethoxy)pyrazin-2- |
| | yl)phenyl)-2-methylpropanamide |
| | 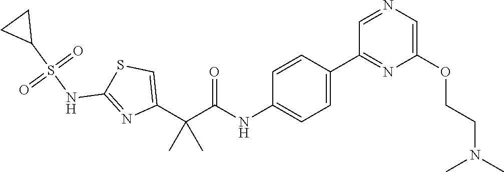 [SMILES: CN(C)CCOc1cncc(-c2ccc(NC(=O)C(C)(C)c3csc(NS(=O)(=O)C4CC4)n3)cc2)n1] |
| T280 | 2-(2-(cyclopropanesulfonamido)thiazol-4- |
| | yl)-2-methyl-N-(4-(3-methylpyrazin-2- |
| | yl)phenyl)propanamide |
| | 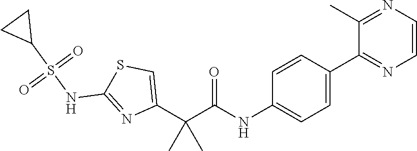 [SMILES: Cc1nccnc1-c1ccc(NC(=O)C(C)(C)c2csc(NS(=O)(=O)C3CC3)n2)cc1] |
| T281 | N-(4-(6-acetamidopyrazin-2-yl)phenyl)-2- |
| | (2-(cyclopropanesulfonamido)thiazol-4-yl)- |
| | 2-methylpropanamide |
| | 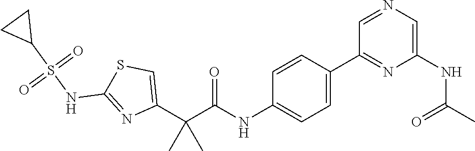 [SMILES: CC(=O)Nc1cncc(-c2ccc(NC(=O)C(C)(C)c3csc(NS(=O)(=O)C4CC4)n3)cc2)n1] |
| T282 | 2-(2-(cyclopropanesulfonamido)thiazol-4- |
| | yl)-N-(4-(5,6-dimethylpyrazin-2-yl)phenyl)- |
| | 2-methylpropanamide |
| | 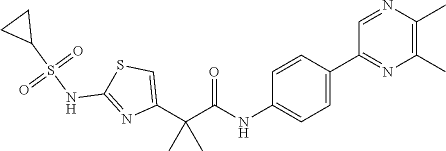 [SMILES: Cc1ncc(-c2ccc(NC(=O)C(C)(C)c3csc(NS(=O)(=O)C4CC4)n3)cc2)nc1C] |
| T283 | 2-(2-(cyclopropanesulfonamido)thiazol-4- |
| | yl)-N-(4-(6-(hydroxymethyl)pyrazin-2- |
| | yl)phenyl)-2-methylpropanamide |
| | 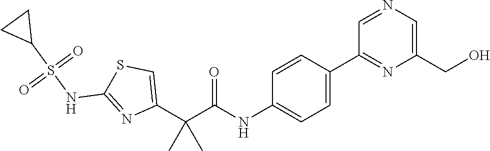 [SMILES: CC(C)(C(=O)Nc1ccc(-c2cncc(CO)n2)cc1)c1csc(NS(=O)(=O)C2CC2)n1] |
| T284 | 2-(2-(cyclopropanesulfonamido)thiazol-4- |
| | yl)-N-(4-(3,6-dimethylpyrazin-2-yl)phenyl)- |
| | 2-methylpropanamide |
| | 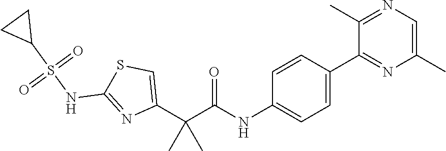 [SMILES: Cc1cnc(C)c(-c2ccc(NC(=O)C(C)(C)c3csc(NS(=O)(=O)C4CC4)n3)cc2)n1] |
| T285 | 2-(2-(cyclopropanesulfonamido)thiazol-4- |
| | yl)-N-(4-(5-methoxypyridin-3-yl)-2- |
| | methylphenyl)-2-methylpropanamide |
| | 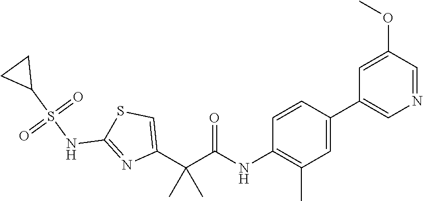 [SMILES: COc1cncc(-c2ccc(NC(=O)C(C)(C)c3csc(NS(=O)(=O)C4CC4)n3)c(C)c2)c1] |
| T286 | N-(4-(5-cyanopyridin-3-yl)-2- |
| | methylphenyl)-2-(2- |
| | (cyclopropanesulfonamido)thiazol-4-yl)-2- |
| | methylpropanamide |
| | 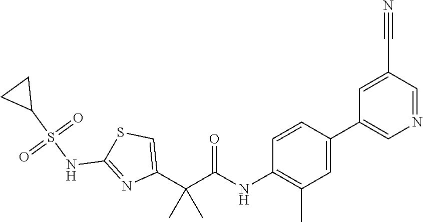 [SMILES: Cc1cc(-c2cncc(C#N)c2)ccc1NC(=O)C(C)(C)c1csc(NS(=O)(=O)C2CC2)n1] |
| T287 | 2-(2-(cyclopropanesulfonamido)thiazol-4- |
| | yl)-N-(4-(5-fluoropyridin-3-yl)-2- |
| | methylphenyl)-2-methylpropanamide |
| | 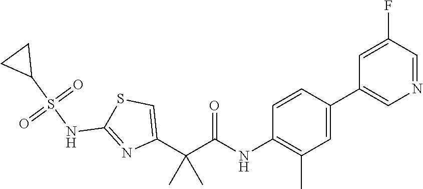 [SMILES: Cc1cc(-c2cncc(F)c2)ccc1NC(=O)C(C)(C)c1csc(NS(=O)(=O)C2CC2)n1] |
| T288 | N-(4-(5-chloropyridin-3-yl)-3- |
| | methylphenyl)-2-(2- |
| | (cyclopropanesulfonamido)thiazol-4-yl)-2- |
| | methylpropanamide |
| | 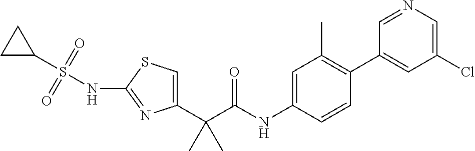 [SMILES: Cc1cc(NC(=O)C(C)(C)c2csc(NS(=O)(=O)C3CC3)n2)ccc1-c1cncc(Cl)c1] |
| T289 | N-(4-(5-cyanopyridin-3-yl)-3- |
| | ethoxyphenyl)-2-(2- |
| | (cyclopropanesulfonamido)thiazol-4-yl)-2- |
| | methylpropanamide |
| | 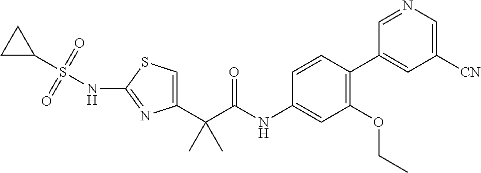 [SMILES: CCOc1cc(NC(=O)C(C)(C)c2csc(NS(=O)(=O)C3CC3)n2)ccc1-c1cncc(C#N)c1] |
| T290 | 2-(2-(cyclopropanesulfonamido)thiazol-4- |
| | yl)-N-(4-(5-ethoxypyridin-3-yl)phenyl)-2- |
| | methylpropanamide |
| | 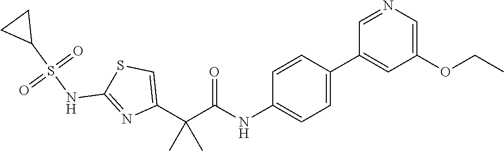 [SMILES: CCOc1cncc(-c2ccc(NC(=O)C(C)(C)c3csc(NS(=O)(=O)C4CC4)n3)cc2)c1] |
| T291 | 2-(2-(cyclopropanesulfonamido)thiazol-4- |
| | yl)-N-(4-(6-cyclopropylpyrazin-2- |
| | yl)phenyl)butanamide |
| | 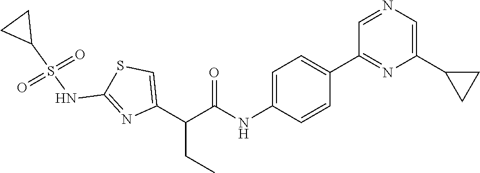 [SMILES: CCC(C(=O)Nc1ccc(-c2cncc(C3CC3)n2)cc1)c1csc(NS(=O)(=O)C2CC2)n1] |
| T292 | 2-(2-(cyclopropanesulfonamido)thiazol-4- |
| | yl)-N-(5-(5-methoxypyridin-3-yl)pyrimidin- |
| | 2-yl)-2-methylpropanamide |
| | 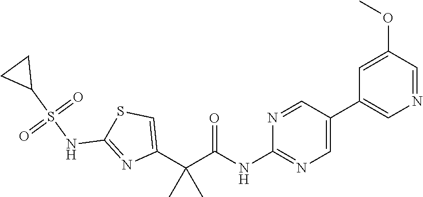 [SMILES: COc1cncc(-c2cnc(NC(=O)C(C)(C)c3csc(NS(=O)(=O)C4CC4)n3)nc2)c1] |
| T293 | 2-(2-(cyclopropanesulfonamido)thiazol-4- |
| | yl)-N-(5-(5-fluoropyridin-3-yl)pyrimidin-2- |
| | yl)-2-methylpropanamide |
| | 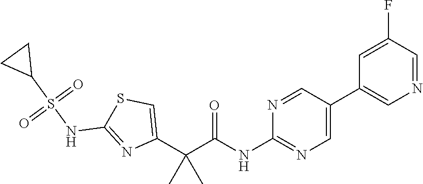 [SMILES: CC(C)(C(=O)Nc1ncc(-c2cncc(F)c2)cn1)c1csc(NS(=O)(=O)C2CC2)n1] |
| T294 | 2-(2-(cyclopropanesulfonamido)thiazol-4- |
| | yl)-2-methyl-N-(5-(5- |
| | (trifluoromethyl)pyridin-3-yl)pyrimidin-2- |
| | yl)propanamide |
| | 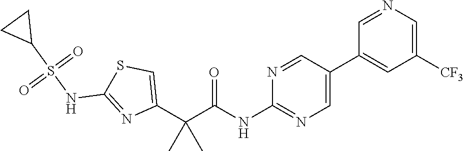 [SMILES: CC(C)(C(=O)Nc1ncc(-c2cncc(C(F)(F)F)c2)cn1)c1csc(NS(=O)(=O)C2CC2)n1] |
| T295 | N-(5-(6-ethoxypyrazin-2-yl)pyridin-2-yl)-2- |
| | methyl-2-(2-((2- |
| | methylpropyl)sulfonamido)thiazol-4- |
| | yl)propanamide |
| | 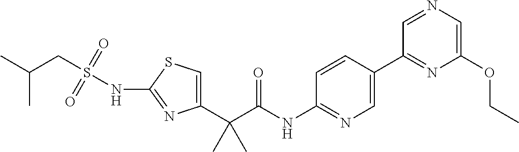 [SMILES: CCOc1cncc(-c2ccc(NC(=O)C(C)(C)c3csc(NS(=O)(=O)CC(C)C)n3)nc2)n1] |
| T296 | N-(4-(6-ethoxypyrazin-2-yl)phenyl)-2- |
| | methyl-2-(2- |
| | ((trifluoromethyl)sulfonamido)thiazol-4- |
| | yl)propanamide |
| | 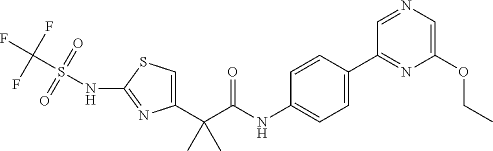 [SMILES: CCOc1cncc(-c2ccc(NC(=O)C(C)(C)c3csc(NS(=O)(=O)C(F)(F)F)n3)cc2)n1] |
| T297 | 2-methyl-2-(2-((1- |
| | methylethyl)sulfonamido)thiazol-4-yl)-N-(4- |
| | (pyridin-3-yl)phenyl)propanamide |
| | 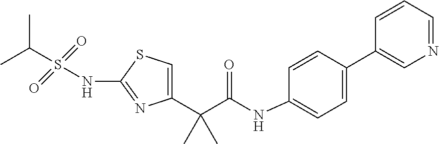 [SMILES: CC(C)S(=O)(=O)Nc1nc(C(C)(C)C(=O)Nc2ccc(-c3cccnc3)cc2)cs1] |
| T298 | N-(4-(6-ethoxypyrazin-2-yl)phenyl)-2- |
| | methyl-2-(2-((1- |
| | methylethyl)sulfonamido)thiazol-4- |
| | yl)propanamide |
| | 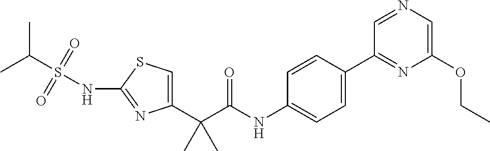 [SMILES: CCOc1cncc(-c2ccc(NC(=O)C(C)(C)c3csc(NS(=O)(=O)C(C)C)n3)cc2)n1] |
| T299 | 2-methyl-2-(2-((1-methylcyclopropane)-1- |
| | sulfonamido)thiazol-4-yl)-N-(4-(pyridin-3- |
| | yl)phenyl)propanamide |
| | 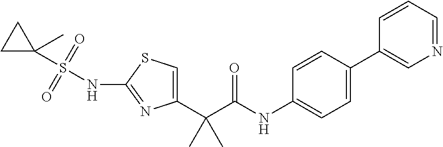 [SMILES: CC(C)(C(=O)Nc1ccc(-c2cccnc2)cc1)c1csc(NS(=O)(=O)C2(C)CC2)n1] |
| T300 | N-(4-(5-chloropyridin-3-yl)phenyl)-2- |
| | methyl-2-(2-((1-methylcyclopropane)-1- |
| | sulfonamido)thiazol-4-yl)propanamide |
| | 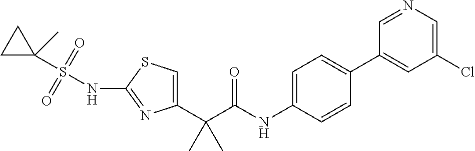 [SMILES: CC(C)(C(=O)Nc1ccc(-c2cncc(Cl)c2)cc1)c1csc(NS(=O)(=O)C2(C)CC2)n1] |
| T301 | N-(4-(6-ethoxypyrazin-2-yl)phenyl)-2- |
| | methyl-2-(2-((1-methylcyclopropane)-1- |
| | sulfonamido)thiazol-4-yl)propanamide |
| | 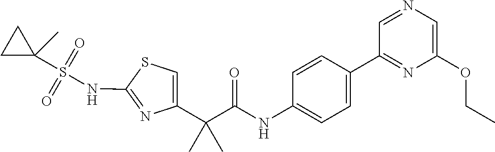 [SMILES: CCOc1cncc(-c2ccc(NC(=O)C(C)(C)c3csc(NS(=O)(=O)C4(C)CC4)n3)cc2)n1] |
| T302 | 2-methyl-2-(2-((1-methylcyclopropane)-1- |
| | sulfonamido)thiazol-4-yl)-N-(4-(6- |
| | (trifluoromethyl)pyrazin-2- |
| | yl)phenyl)propanamide |
| | 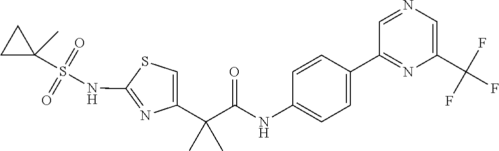 [SMILES: CC(C)(C(=O)Nc1ccc(-c2cncc(C(F)(F)F)n2)cc1)c1csc(NS(=O)(=O)C2(C)CC2)n1] |
| T303 | 2-(2-((1,1- |
| | dimethylethyl)sulfonamido)thiazol-4-yl)-2- |
| | methyl-N-(4-(pyridin-3- |
| | yl)phenyl)propanamide |
| | 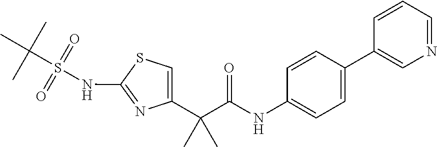 [SMILES: CC(C)(C(=O)Nc1ccc(-c2cccnc2)cc1)c1csc(NS(=O)(=O)C(C)(C)C)n1] |
| T304 | 2-(2-((1,1- |
| | dimethylethyl)sulfonamido)thiazol-4-yl)-N- |
| | (4-(6-ethoxypyrazin-2-yl)phenyl)-2- |
| | methylpropanamide |
| | 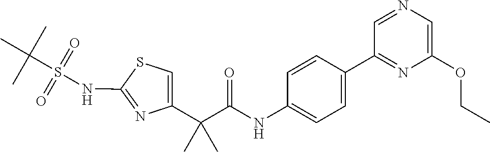 [SMILES: CCOc1cncc(-c2ccc(NC(=O)C(C)(C)c3csc(NS(=O)(=O)C(C)(C)C)n3)cc2)n1] |
| T305 | 2-(2-((1,1- |
| | dimethylethyl)sulfonamido)thiazol-4-yl)-2- |
| | methyl-N-(4-(6-(trifluoromethyl)pyrazin-2- |
| | yl)phenyl)propanamide |
| | 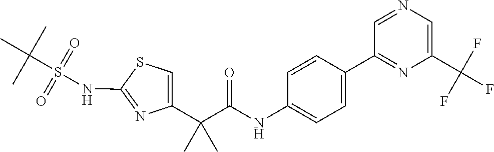 [SMILES: CC(C)(C(=O)Nc1ccc(-c2cncc(C(F)(F)F)n2)cc1)c1csc(NS(=O)(=O)C(C)(C)C)n1] |
| T306 | 2-(2-(cyclobutanesulfonamido)thiazol-4-yl)- |
| | 2-methyl-N-(4-(pyridin-3- |
| | yl)phenyl)propanamide |
| | 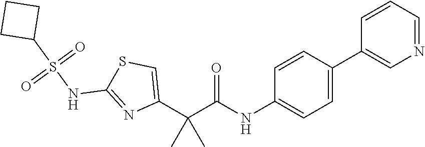 [SMILES: CC(C)(C(=O)Nc1ccc(-c2cccnc2)cc1)c1csc(NS(=O)(=O)C2CCC2)n1] |
| T307 | 2-(2-(cyclobutanesulfonamido)thiazol-4-yl)- |
| | N-(4-(6-ethoxypyrazin-2-yl)phenyl)-2- |
| | methylpropanamide |
| | 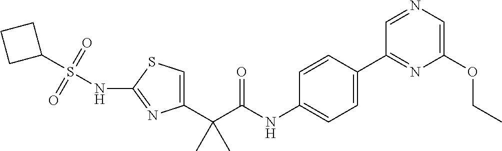 [SMILES: CCOc1cncc(-c2ccc(NC(=O)C(C)(C)c3csc(NS(=O)(=O)C4CCC4)n3)cc2)n1] |
| T308 | 2-(2-(cyclobutanesulfonamido)thiazol-4-yl)- |
| | 2-methyl-N-(4-(6-(trifluoromethyl)pyrazin- |
| | 2-yl)phenyl)propanamide |
| | 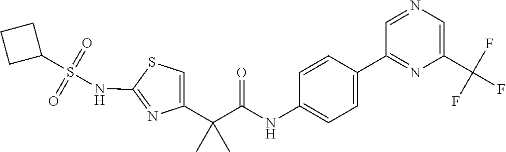 [SMILES: CC(C)(C(=O)Nc1ccc(-c2cncc(C(F)(F)F)n2)cc1)c1csc(NS(=O)(=O)C2CCC2)n1] |
| T309 | N-(4-(5-cyanopyridin-3-yl)phenyl)-2-(2- |
| | (cyclopropanesulfonamido)thiazol-4-yl)- |
| | N,2-dimethylpropanamide |
| | 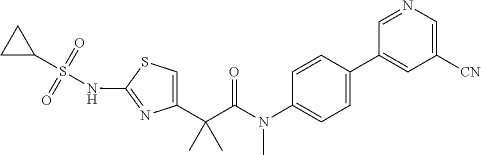 [SMILES: CN(C(=O)C(C)(C)c1csc(NS(=O)(=O)C2CC2)n1)c1ccc(-c2cncc(C#N)c2)cc1] |
| T310 | 2-(2-(cyclopropanesulfonamido)thiazol-4- |
| | yl)-N,2-dimethyl-N-(4-(6- |
| | (trifluoromethyl)pyrazin-2- |
| | yl)phenyl)propanamide |
| | 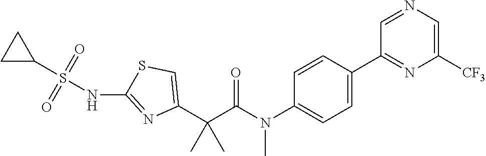 [SMILES: CN(C(=O)C(C)(C)c1csc(NS(=O)(=O)C2CC2)n1)c1ccc(-c2cncc(C(F)(F)F)n2)cc1] |
| T311 | 2-methyl-2-(2-((2- |
| | methylpropyl)sulfonamido)thiazol-4-yl)-N- |
| | (4-(pyridin-3-yl)phenyl)propanamide |
| | 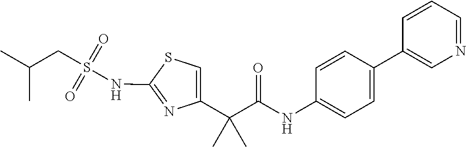 [SMILES: CC(C)CS(=O)(=O)Nc1nc(C(C)(C)C(=O)Nc2ccc(-c3cccnc3)cc2)cs1] |
| T312 | N-(4-(6-ethoxypyrazin-2-yl)phenyl)-2- |
| | methyl-2-(2-((2- |
| | methylpropyl)sulfonamido)thiazol-4- |
| | yl)propanamide |
| | 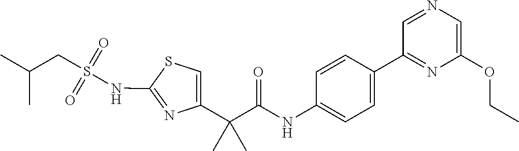 [SMILES: CCOc1cncc(-c2ccc(NC(=O)C(C)(C)c3csc(NS(=O)(=O)CC(C)C)n3)cc2)n1] |
| T313 | 2-methyl-2-(2-((2- |
| | methylpropyl)sulfonamido)thiazol-4-yl)-N- |
| | (4-(6-(trifluoromethyl)pyrazin-2- |
| | yl)phenyl)propanamide |
| | 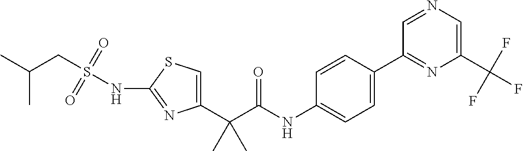 [SMILES: CC(C)CS(=O)(=O)Nc1nc(C(C)(C)C(=O)Nc2ccc(-c3cncc(C(F)(F)F)n3)cc2)cs1] |
| T314 | N-(4-(5-chloropyridin-3-yl)phenyl)-2-(2- |
| | (cyclopropanesulfonamido)-5- |
| | methylthiazol-4-yl)-2-methylpropanamide |
| | 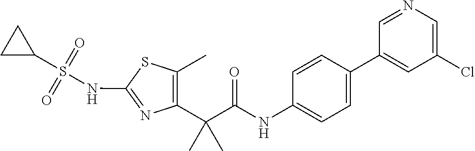 [SMILES: Cc1sc(NS(=O)(=O)C2CC2)nc1C(C)(C)C(=O)Nc1ccc(-c2cncc(Cl)c2)cc1] |
| T315 | 2-(2-(cyclopropanesulfonamido)thiazol-4- |
| | yl)-N-methyl-N-(4-(pyridin-3- |
| | yl)phenyl)butanamide |
| | 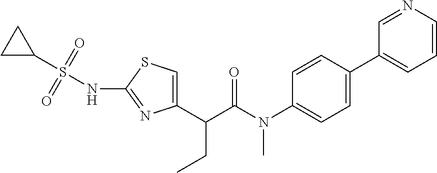 [SMILES: CCC(C(=O)N(C)c1ccc(-c2cccnc2)cc1)c1csc(NS(=O)(=O)C2CC2)n1] |
| T316 | N-(4-(5-cyanopyridin-3-yl)phenyl)-2-(2- |
| | (cyclopropanesulfonamido)thiazol-4-yl)-N- |
| | methylbutanamide |
| | 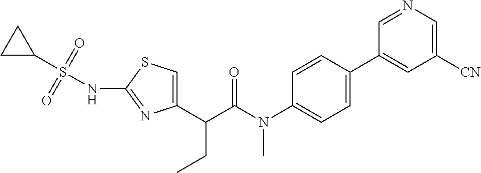 [SMILES: CCC(C(=O)N(C)c1ccc(-c2cncc(C#N)c2)cc1)c1csc(NS(=O)(=O)C2CC2)n1] |
| T317 | 2-(2-(cyclopropanesulfonamido)-5- |
| | methylthiazol-4-yl)-N-(4-(6-ethoxypyrazin- |
| | 2-yl)phenyl)-2-methylpropanamide |
| | 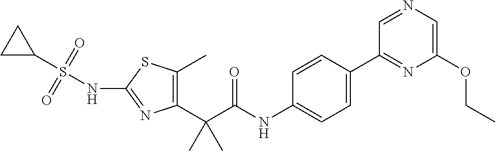 [SMILES: CCOc1cncc(-c2ccc(NC(=O)C(C)(C)c3nc(NS(=O)(=O)C4CC4)sc3C)cc2)n1] |
| T318 | N-(4-(5-chloropyridin-3-yl)phenyl)-2-(2- |
| | (cyclopropanesulfonamido)thiazol-4-yl)- |
| | N,2-dimethylpropanamide |
| | 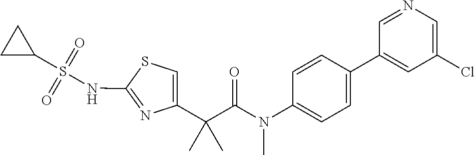 [SMILES: CN(C(=O)C(C)(C)c1csc(NS(=O)(=O)C2CC2)n1)c1ccc(-c2cncc(Cl)c2)cc1] |
| T319 | 2-(2-(cyclopropanesulfonamido)-5- |
| | methylthiazol-4-yl)-2-methyl-N-(4-(pyridin- |
| | 3-yl)phenyl)propanamide |
| | 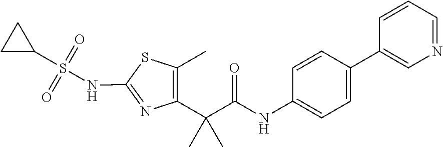 [SMILES: Cc1sc(NS(=O)(=O)C2CC2)nc1C(C)(C)C(=O)Nc1ccc(-c2cccnc2)cc1] |
| T320 | N-(4-(5-cyanopyridin-3-yl)-2,6- |
| | dimethylphenyl)-2-(2- |
| | (cyclopropanesulfonamido)thiazol-4-yl)-2- |
| | methylpropanamide |
| | 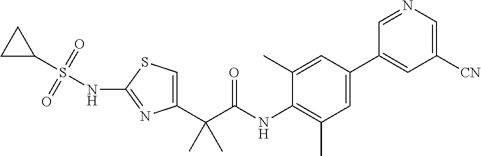 [SMILES: Cc1cc(-c2cncc(C#N)c2)cc(C)c1NC(=O)C(C)(C)c1csc(NS(=O)(=O)C2CC2)n1] |
| T321 | N-(4-(5-chloropyridin-3-yl)-2,6- |
| | dimethylphenyl)-2-(2- |
| | (cyclopropanesulfonamido)thiazol-4-yl)-2- |
| | methylpropanamide |
| | 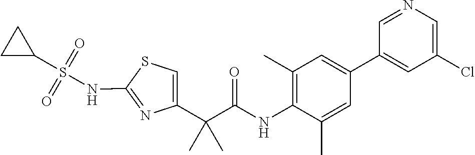 [SMILES: Cc1cc(-c2cncc(Cl)c2)cc(C)c1NC(=O)C(C)(C)c1csc(NS(=O)(=O)C2CC2)n1] |
| T322 | N-(4-(5-cyanopyridin-3-yl)-3- |
| | methylphenyl)-2-(2- |
| | (cyclopropanesulfonamido)thiazol-4-yl)-2- |
| | methylpropanamide |
| | 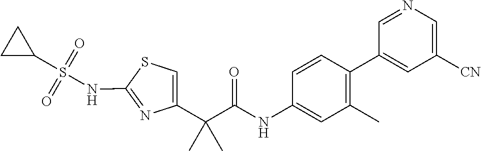 [SMILES: Cc1cc(NC(=O)C(C)(C)c2csc(NS(=O)(=O)C3CC3)n2)ccc1-c1cncc(C#N)c1] |
| T422 | 2-(2-(cyclopropanesulfonamido)thiazol-4- |
| | yl)-N-(5-(6-ethoxypyrazin-2-yl)pyridin-2-yl)- |
| | 2,2-difluoroacetamide |
| | 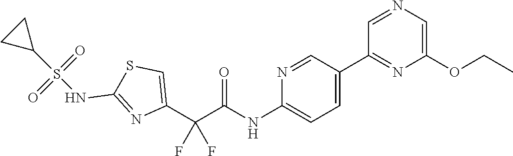 [SMILES: CCOc1cncc(-c2ccc(NC(=O)C(F)(F)c3csc(NS(=O)(=O)C4CC4)n3)nc2)n1] |
| T423 | 2-(2-(cyclopropanesulfonamido)thiazol-4- |
| | yl)-N-(4-(6-ethoxypyrazin-2-yl)-2- |
| | fluorophenyl)acetamide |
| | 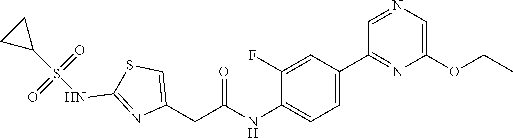 [SMILES: CCOc1cncc(-c2ccc(NC(=O)Cc3csc(NS(=O)(=O)C4CC4)n3)c(F)c2)n1] |
| T424 | 2-(2-(cyclopropanesulfonamido)thiazol-4- |
| | yl)-N-(4-(6-ethoxypyrazin-2- |
| | yl)phenyl)acetamide |
| | 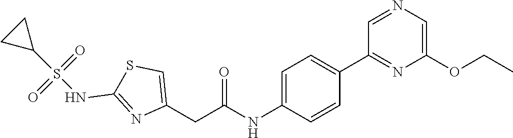 [SMILES: CCOc1cncc(-c2ccc(NC(=O)Cc3csc(NS(=O)(=O)C4CC4)n3)cc2)n1] |
| T425 | 2-methyl-2-(2-(methylsulfonamido)thiazol- |
| | 4-yl)-N-(5-(6-(trifluoromethyl)pyrazin-2- |
| | yl)pyridin-2-yl)propanamide |
| | 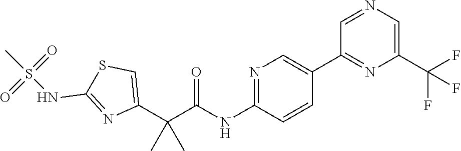 [SMILES: CC(C)(C(=O)Nc1ccc(-c2cncc(C(F)(F)F)n2)cn1)c1csc(NS(C)(=O)=O)n1] |
| T426 | N-(2-fluoro-4-(5-(trifluoromethyl)pyridin-3- |
| | yl)phenyl)-2-methyl-2-(2- |
| | (methylsulfonamido)thiazol-4- |
| | yl)propanamide |
| | 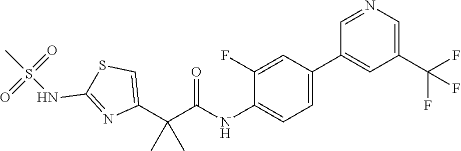 [SMILES: CC(C)(C(=O)Nc1ccc(-c2cncc(C(F)(F)F)c2)cc1F)c1csc(NS(C)(=O)=O)n1] |
| T427 | 2-(2- |
| | ((cyclopropylmethyl)sulfonamido)thiazol-4- |
| | yl)-N-(2-fluoro-4-(5-(trifluoromethyl)pyridin- |
| | 3-yl)phenyl)-2-methylpropanamide |
| | 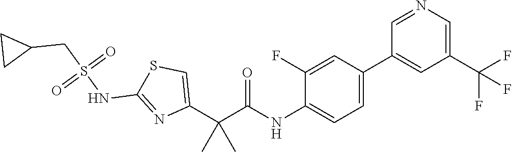 [SMILES: CC(C)(C(=O)Nc1ccc(-c2cncc(C(F)(F)F)c2)cc1F)c1csc(NS(=O)(=O)CC2CC2)n1] |
| T428 | N-(4-(5-chloro-4-methylpyridin-3- |
| | yl)phenyl)-2-(2- |
| | (cyclopropanesulfonamido)thiazol-4-yl)-2- |
| | methylpropanamide |
| | 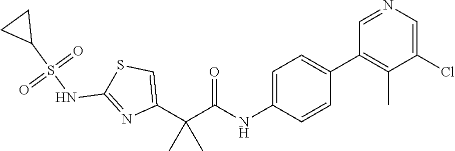 [SMILES: Cc1c(Cl)cncc1-c1ccc(NC(=O)C(C)(C)c2csc(NS(=O)(=O)C3CC3)n2)cc1] |
| T429 | N-(4-(6-ethoxypyrazin-2-yl)-2- |
| | (trifluoromethyl)phenyl)-2-methyl-2-(2- |
| | (methylsulfonamido)thiazol-4- |
| | yl)propanamide |
| | 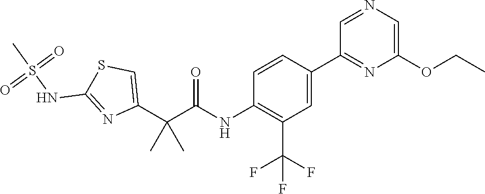 [SMILES: CCOc1cncc(-c2ccc(NC(=O)C(C)(C)c3csc(NS(C)(=O)=O)n3)c(C(F)(F)F)c2)n1] |
| T430 | 2-(2- |
| | ((cyclopropylmethyl)sulfonamido)thiazol-4- |
| | yl)-N-(2-fluoro-4-(6- |
| | (trifluoromethyl)pyrazin-2-yl)phenyl)-2- |
| | methylpropanamide |
| | 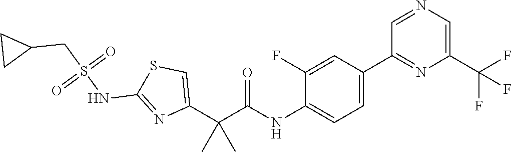 [SMILES: CC(C)(C(=O)Nc1ccc(-c2cncc(C(F)(F)F)n2)cc1F)c1csc(NS(=O)(=O)CC2CC2)n1] |
| T431 | N-(4-(6-ethoxypyrazin-2-yl)-2- |
| | fluorophenyl)-2-methyl-2-(2- |
| | (methylsulfonamido)thiazol-4- |
| | yl)propanamide |
| | 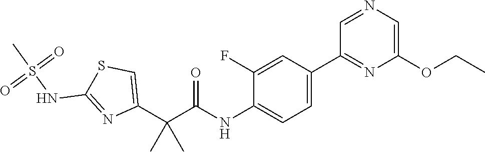 [SMILES: CCOc1cncc(-c2ccc(NC(=O)C(C)(C)c3csc(NS(C)(=O)=O)n3)c(F)c2)n1] |
| T432 | N-(4-(6-ethoxypyrazin-2-yl)-2- |
| | fluorophenyl)-2-(2-((2- |
| | methoxyethyl)sulfonamido)thiazol-4-yl)-2- |
| | methylpropanamide |
| | 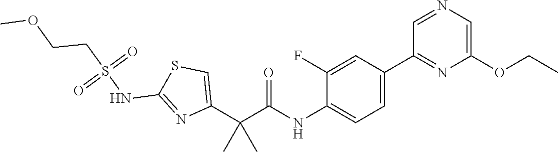 [SMILES: CCOc1cncc(-c2ccc(NC(=O)C(C)(C)c3csc(NS(=O)(=O)CCOC)n3)c(F)c2)n1] |
| T433 | 2-(2- |
| | ((cyclopropylmethyl)sulfonamido)thiazol-4- |
| | yl)-N-(4-(6-ethoxypyrazin-2-yl)-2- |
| | fluorophenyl)-2-methylpropanamide |
| | 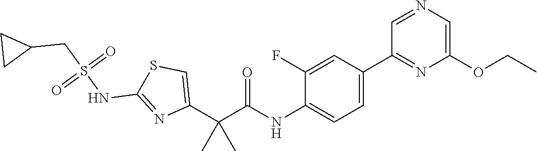 [SMILES: CCOc1cncc(-c2ccc(NC(=O)C(C)(C)c3csc(NS(=O)(=O)CC4CC4)n3)c(F)c2)n1] |
| T434 | N-(4-(6-ethoxypyrazin-2-yl)phenyl)-2- |
| | methyl-2-(2-(methylsulfonamido)thiazol-4- |
| | yl)propanamide |
| | 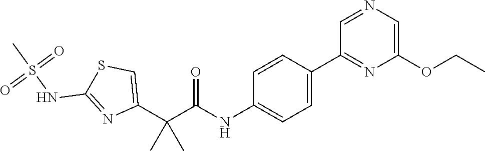 [SMILES: CCOc1cncc(-c2ccc(NC(=O)C(C)(C)c3csc(NS(C)(=O)=O)n3)cc2)n1] |
| T435 | 2-(2-(cyclopropanesulfonamido)thiazol-4- |
| | yl)-N-(5-(6-ethoxypyrazin-2-yl)-3- |
| | fluoropyridin-2-yl)-4-methoxybutanamide |
| | 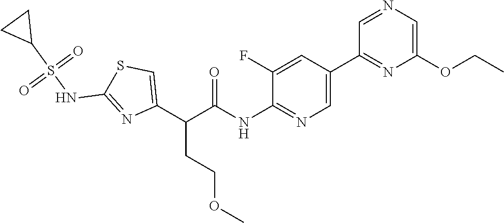 [SMILES: CCOc1cncc(-c2cnc(NC(=O)C(CCOC)c3csc(NS(=O)(=O)C4CC4)n3)c(F)c2)n1] |
| T436 | 2-(2-(cyclopropanesulfonamido)thiazol-4- |
| | yl)-N-(5-(6-ethoxypyrazin-2-yl)pyridin-2-yl)- |
| | 4-methoxybutanamide |
| | 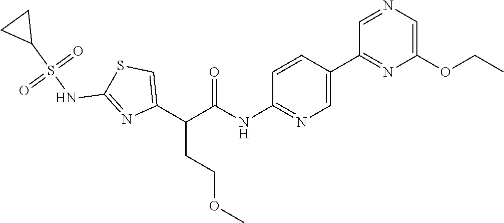 [SMILES: CCOc1cncc(-c2ccc(NC(=O)C(CCOC)c3csc(NS(=O)(=O)C4CC4)n3)nc2)n1] |
| T437 | 2-(2-(cyclopropanesulfonamido)thiazol-4- |
| | yl)-4-methoxy-N-(4-(6- |
| | (trifluoromethyl)pyrazin-2- |
| | yl)phenyl)butanamide |
| | 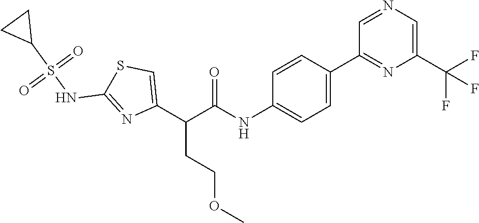 [SMILES: COCCC(C(=O)Nc1ccc(-c2cncc(C(F)(F)F)n2)cc1)c1csc(NS(=O)(=O)C2CC2)n1] |
| T438 | 2-(2-(cyclopropanesulfonamido)thiazol-4- |
| | yl)-N-(5-(6-ethoxypyrazin-2-yl)-3- |
| | methylpyridin-2-yl)butanamide |
| | 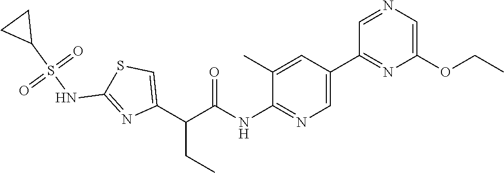 [SMILES: CCOc1cncc(-c2cnc(NC(=O)C(CC)c3csc(NS(=O)(=O)C4CC4)n3)c(C)c2)n1] |
| T439 | N-(2-chloro-4-(6-ethoxypyrazin-2- |
| | yl)phenyl)-2-(2- |
| | (cyclopropanesulfonamido)thiazol-4- |
| | yl)butanamide |
| | 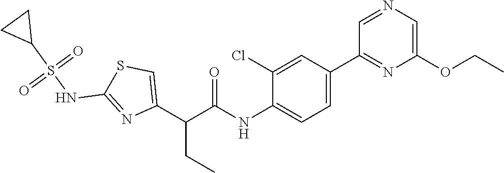 [SMILES: CCOc1cncc(-c2ccc(NC(=O)C(CC)c3csc(NS(=O)(=O)C4CC4)n3)c(Cl)c2)n1] |
| T440 | N-(2-cyano-4-(6-ethoxypyrazin-2- |
| | yl)phenyl)-2-(2- |
| | (cyclopropanesulfonamido)thiazol-4- |
| | yl)butanamide |
| | 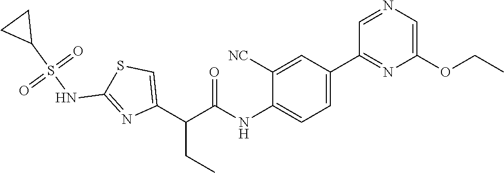 [SMILES: CCOc1cncc(-c2ccc(NC(=O)C(CC)c3csc(NS(=O)(=O)C4CC4)n3)c(C#N)c2)n1] |
| T441 | 2-(2-(cyclopropanesulfonamido)thiazol-4- |
| | yl)-N-(4-(6-ethoxypyrazin-2-yl)-2- |
| | methylphenyl)butanamide |
| | 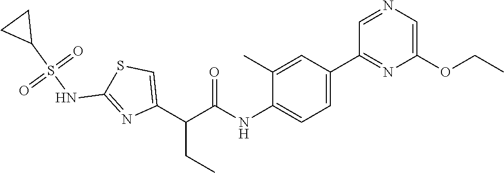 [SMILES: CCOc1cncc(-c2ccc(NC(=O)C(CC)c3csc(NS(=O)(=O)C4CC4)n3)c(C)c2)n1] |
| T442 | 2-(2-(cyclopropanesulfonamido)thiazol-4- |
| | yl)-N-(4-(6-ethoxypyrazin-2-yl)-2- |
| | (trifluoromethoxy)phenyl)butanamide |
| | 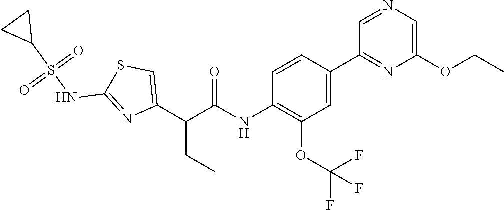 [SMILES: CCOc1cncc(-c2ccc(NC(=O)C(CC)c3csc(NS(=O)(=O)C4CC4)n3)c(OC(F)(F)F)c2)n1] |
| T443 | 2-(2-(cyclopropanesulfonamido)thiazol-4- |
| | yl)-N-(4-(6-ethoxypyrazin-2-yl)-2- |
| | methoxyphenyl)butanamide |
| | 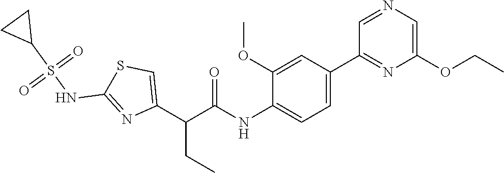 [SMILES: CCOc1cncc(-c2ccc(NC(=O)C(CC)c3csc(NS(=O)(=O)C4CC4)n3)c(OC)c2)n1] |
| T445 | 2-(2-(cyclopropanesulfonamido)thiazol-4- |
| | yl)-N-(5-(6-ethoxypyrazin-2-yl)-3- |
| | fluoropyridin-2-yl)-2-methoxyacetamide |
| | 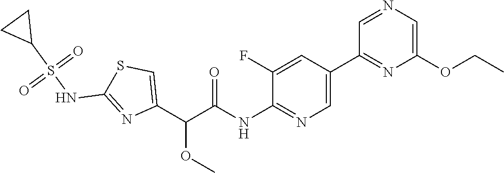 [SMILES: CCOc1cncc(-c2cnc(NC(=O)C(OC)c3csc(NS(=O)(=O)C4CC4)n3)c(F)c2)n1] |
| T446 | 2-(2-(cyclopropanesulfonamido)thiazol-4- |
| | yl)-N-(5-(6-ethoxypyrazin-2-yl)pyridin-2-yl)- |
| | 2-methoxyacetamide |
| | 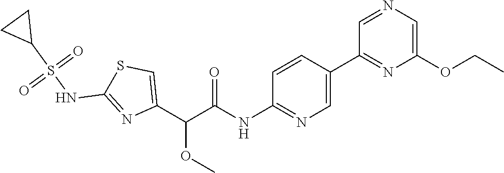 [SMILES: CCOc1cncc(-c2ccc(NC(=O)C(OC)c3csc(NS(=O)(=O)C4CC4)n3)nc2)n1] |
| T447 | 2-(2-(cyclopropanesulfonamido)thiazol-4- |
| | yl)-N-(4-(5-fluoropyridin-3-yl)-2- |
| | (trifluoromethyl)phenyl)-2- |
| | methoxyacetamide |
| | 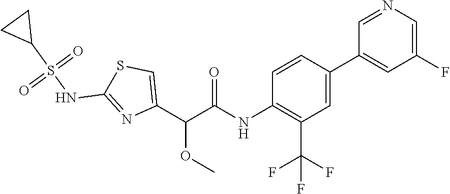 [SMILES: COC(C(=O)Nc1ccc(-c2cncc(F)c2)cc1C(F)(F)F)c1csc(NS(=O)(=O)C2CC2)n1] |
| T448 | 2-(2-(cyclopropanesulfonamido)thiazol-4- |
| | yl)-N-(2-fluoro-4-(5-(trifluoromethyl)pyridin- |
| | 3-yl)phenyl)-2-methoxyacetamide |
| | 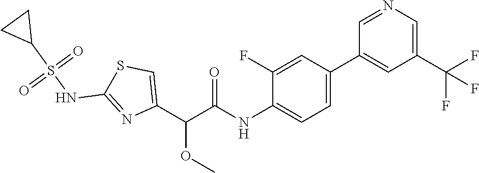 [SMILES: COC(C(=O)Nc1ccc(-c2cncc(C(F)(F)F)c2)cc1F)c1csc(NS(=O)(=O)C2CC2)n1] |
| T449 | 2-(2-(cyclopropanesulfonamido)thiazol-4- |
| | yl)-N-(4-(6-ethoxypyrazin-2-yl)-2- |
| | (trifluoromethyl)phenyl)-2- |
| | methoxyacetamide |
| | 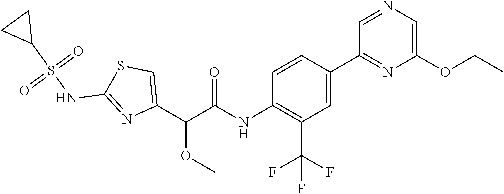 [SMILES: CCOc1cncc(-c2ccc(NC(=O)C(OC)c3csc(NS(=O)(=O)C4CC4)n3)c(C(F)(F)F)c2)n1] |
| T450 | N-(2-chloro-4-(6-ethoxypyrazin-2- |
| | yl)phenyl)-2-(2- |
| | (cyclopropanesulfonamido)thiazol-4-yl)-2- |
| | methoxyacetamide |
| | 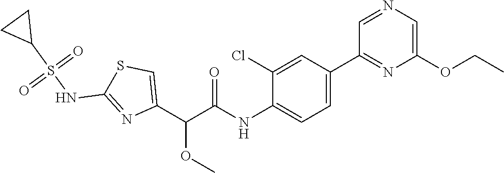 [SMILES: CCOc1cncc(-c2ccc(NC(=O)C(OC)c3csc(NS(=O)(=O)C4CC4)n3)c(Cl)c2)n1] |
| T451 | N-(2-cyano-4-(6-ethoxypyrazin-2- |
| | yl)phenyl)-2-(2- |
| | (cyclopropanesulfonamido)thiazol-4-yl)-2- |
| | methoxyacetamide |
| | 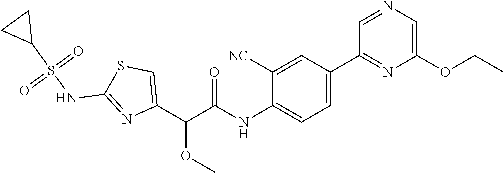 [SMILES: CCOc1cncc(-c2ccc(NC(=O)C(OC)c3csc(NS(=O)(=O)C4CC4)n3)c(C#N)c2)n1] |
| T452 | N-(2-fluoro-4-(6-(trifluoromethyl)pyrazin-2- |
| | yl)phenyl)-2-methoxy-2-(2- |
| | (methylsulfonamido)thiazol-4-yl)acetamide |
| | 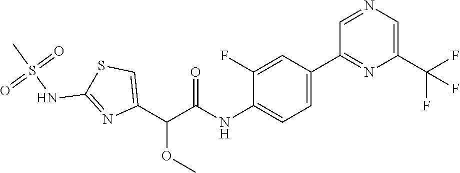 [SMILES: COC(C(=O)Nc1ccc(-c2cncc(C(F)(F)F)n2)cc1F)c1csc(NS(C)(=O)=O)n1] |
| T453 | 2-(2-(cyclopropanesulfonamido)thiazol-4- |
| | yl)-N-(4-(6-ethoxypyrazin-2-yl)-2,6- |
| | difluorophenyl)-2-methoxyacetamide |
| | 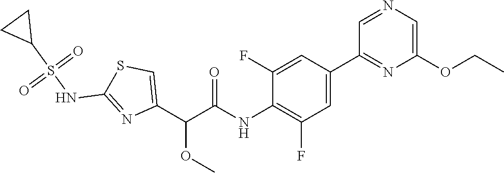 [SMILES: CCOc1cncc(-c2cc(F)c(NC(=O)C(OC)c3csc(NS(=O)(=O)C4CC4)n3)c(F)c2)n1] |
| T454 | 2-(2-(cyclopropanesulfonamido)thiazol-4- |
| | yl)-N-(4-(6-ethoxypyrazin-2-yl)-2- |
| | (trifluoromethoxy)phenyl)-2- |
| | methoxyacetamide |
| | 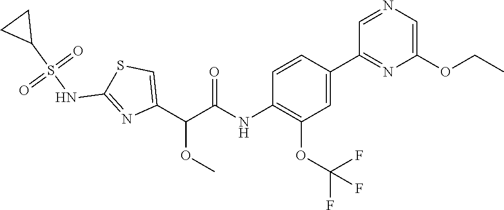 [SMILES: CCOc1cncc(-c2ccc(NC(=O)C(OC)c3csc(NS(=O)(=O)C4CC4)n3)c(OC(F)(F)F)c2)n1] |
| T455 | N-(4-(6-ethoxypyrazin-2-yl)phenyl)-2- |
| | methoxy-2-(2-(methylsulfonamido)thiazol- |
| | 4-yl)acetamide |
| | 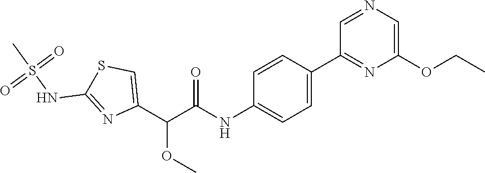 [SMILES: CCOc1cncc(-c2ccc(NC(=O)C(OC)c3csc(NS(C)(=O)=O)n3)cc2)n1] |
| T456 | Single enantiomer—stereochemistry |
| | unassigned |
| | 2-(2-(cyclopropanesulfonamido)thiazol-4- |
| | yl)-N-(5-(6-ethoxypyrazin-2-yl)-3- |
| | fluoropyridin-2-yl)butanamide |
| | 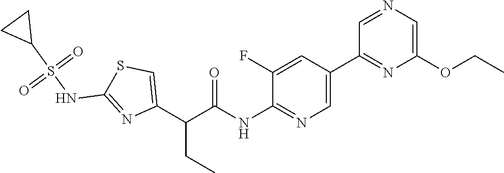 [SMILES: CCOc1cncc(-c2cnc(NC(=O)C(CC)c3csc(NS(=O)(=O)C4CC4)n3)c(F)c2)n1] |
| T457 | Single enantiomer—stereochemistry |
| | unassigned |
| | 2-(2-(cyclopropanesulfonamido)thiazol-4- |
| | yl)-N-(5-(6-ethoxypyrazin-2-yl)-3- |
| | fluoropyridin-2-yl)butanamide |
| | 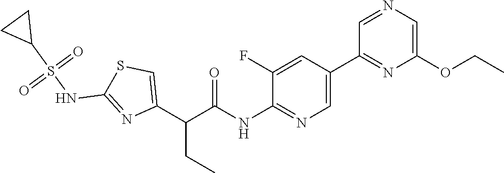 [SMILES: CCOc1cncc(-c2cnc(NC(=O)C(CC)c3csc(NS(=O)(=O)C4CC4)n3)c(F)c2)n1] |
| T458 | Single enantiomer—stereochemistry |
| | unassigned |
| | 2-(2-(cyclopropanesulfonamido)thiazol-4- |
| | yl)-N-(2-fluoro-4-(6- |
| | (trifluoromethyl)pyrazin-2-yl)phenyl)-2- |
| | methoxyacetamide |
| | 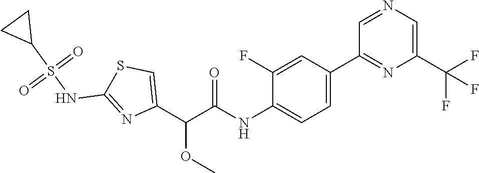 [SMILES: COC(C(=O)Nc1ccc(-c2cncc(C(F)(F)F)n2)cc1F)c1csc(NS(=O)(=O)C2CC2)n1] |
| T459 | Single enantiomer—stereochemistry |
| | unassigned |
| | 2-(2-(cyclopropanesulfonamido)thiazol-4- |
| | yl)-N-(2-fluoro-4-(6- |
| | (trifluoromethyl)pyrazin-2-yl)phenyl)-2- |
| | methoxyacetamide |
| | 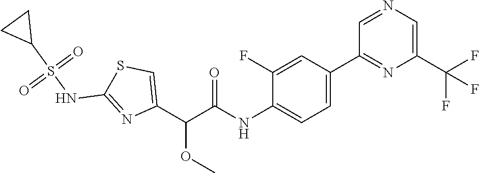 [SMILES: COC(C(=O)Nc1ccc(-c2cncc(C(F)(F)F)n2)cc1F)c1csc(NS(=O)(=O)C2CC2)n1] |
| T460 | 4-(2-(cyclopropanesulfonamido)thiazol-4- |
| | yl)-N-(4-(6-ethoxypyrazin-2-yl)-2- |
| | fluorophenyl)tetrahydro-2H-pyran-4- |
| | carboxamide |
| | 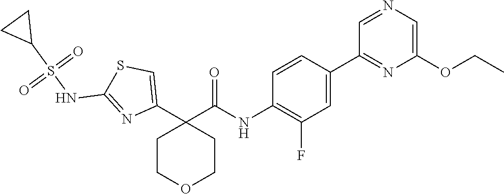 [SMILES: CCOc1cncc(-c2ccc(NC(=O)C3(c4csc(NS(=O)(=O)C5CC5)n4)CCOCC3)c(F)c2)n1] |
| T461 | 4-(2-(cyclopropanesulfonamido)thiazol-4- |
| | yl)-N-(5-(6-(trifluoromethyl)pyrazin-2- |
| | yl)pyridin-2-yl)tetrahydro-2H-pyran-4- |
| | carboxamide |
| | 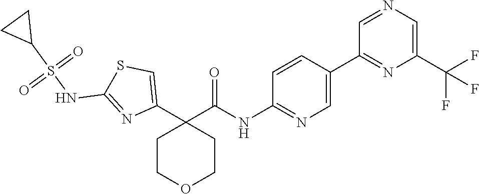 [SMILES: O=C(Nc1ccc(-c2cncc(C(F)(F)F)n2)cn1)C1(c2csc(NS(=O)(=O)C3CC3)n2)CCOCC1] |
| T462 | 4-(2-(cyclopropanesulfonamido)thiazol-4- |
| | yl)-N-(5-(6-ethoxypyrazin-2-yl)pyridin-2- |
| | yl)tetrahydro-2H-pyran-4-carboxamide |
| | 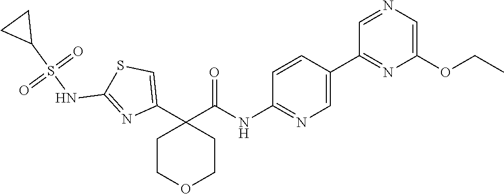 [SMILES: CCOc1cncc(-c2ccc(NC(=O)C3(c4csc(NS(=O)(=O)C5CC5)n4)CCOCC3)nc2)n1] |
| T463 | N-(4-(1-(4-(5-methoxypyridin-3-yl)phenyl)- |
| | 2-oxopyrrolidin-3-yl)thiazol-2- |
| | yl)cyclopropanesulfonamide |
| | 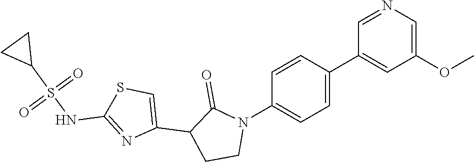 [SMILES: COc1cncc(-c2ccc(N3CCC(c4csc(NS(=O)(=O)C5CC5)n4)C3=O)cc2)c1] |
| T464 | 2-(2-(cyclopropanesulfonamido)thiazol-4- |
| | yl)-2-methyl-N-(5-(6-methylpyrazin-2- |
| | yl)pyridin-2-yl)propanamide |
| | 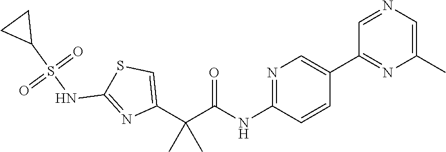 [SMILES: Cc1cncc(-c2ccc(NC(=O)C(C)(C)c3csc(NS(=O)(=O)C4CC4)n3)nc2)n1] |
| T465 | N-(4-(6-cyanopyrazin-2-yl)-2- |
| | methylphenyl)-2-(2- |
| | (cyclopropanesulfonamido)thiazol-4-yl)-2- |
| | methylpropanamide |
| | 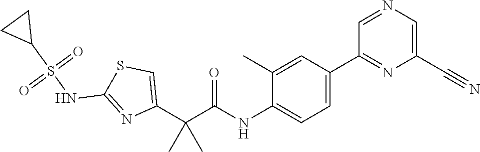 [SMILES: Cc1cc(-c2cncc(C#N)n2)ccc1NC(=O)C(C)(C)c1csc(NS(=O)(=O)C2CC2)n1] |

Method 2c: Telescoped Boronate Formation and Suzuki Coupling on Sulfonamide Scaffold

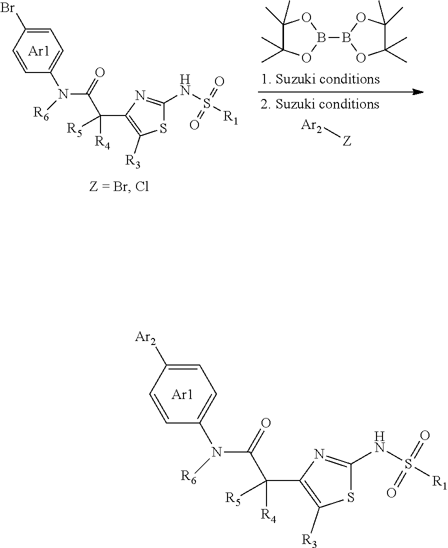

A suspension of Ar1-Br (1 eq), bispin (1.1 eq.) and KOAc (2 eq) in dioxane (50 volumes) was degassed (N₂, 5 mins, ×3) then charged with PdCl₂(dppo)-DCM adduct (5 mol %) and again degassed (N₂, 5 mins, ×3). The reaction mixture was heated to 90° C. for 1 hr and then the reaction was allowed to cool to RT. Ar2-Z (1 eq) and 2M K₂CO₃ (aq, 2 eq) were added and the reaction was then heated to 90° C. for 18 hrs. The reaction was allowed to cool to RT, an aqueous work up was performed and the crude compound was purified by normal phase chromatography.
2-Amino-2-(2-(cyclopropanesulfonamido)thiazol-4-yl)-N-(4-(6-ethoxypyrazin-2-yl)phenyl)acetamide T325

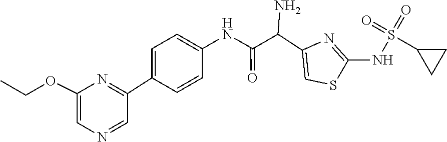 [SMILES: CCOc1cncc(-c2ccc(NC(=O)C(N)c3csc(NS(=O)(=O)C4CC4)n3)cc2)n1]

2-acetamido-2-(2-(cyclopropanesulfonamido)thiazol-4-yl)-N-(4-(6-ethoxypyrazin-2-yl)phenyl)acetamide T326

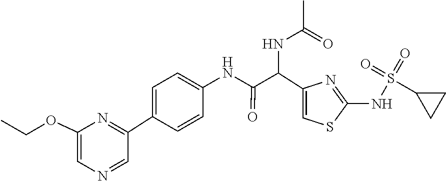 [SMILES: CCOc1cncc(-c2ccc(NC(=O)C(NC(C)=O)c3csc(NS(=O)(=O)C4CC4)n3)cc2)n1]

methyl (1-(2-(cyclopropanesulfonamido)thiazol-4-yl)-2-((4-(6-ethoxypyrazin-2-yl)phenyl)amino)-2-oxoethyl)carbamate T327

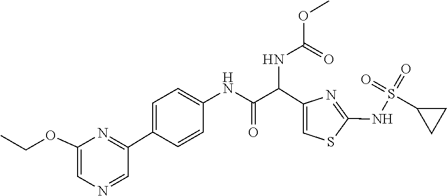 [SMILES: CCOc1cncc(-c2ccc(NC(=O)C(NC(=O)OC)c3csc(NS(=O)(=O)C4CC4)n3)cc2)n1]

2-(2-(cyclopropanesulfonamido)thiazol-4-yl)-2-(dimethylamino)-N-(4-(6-ethoxypyrazin-2-yl)phenyl)acetamide T328

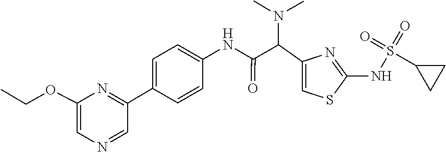 [SMILES: CCOc1cncc(-c2ccc(NC(=O)C(c3csc(NS(=O)(=O)C4CC4)n3)N(C)C)cc2)n1]

2-(2-(cyclopropanesulfonamido)thiazol-4-yl)-N-(4-(6-ethoxypyrazin-2-yl)phenyl)-4-hydroxybutanamide T329

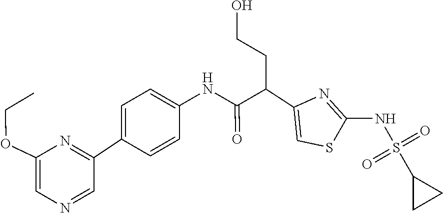 [SMILES: CCOc1cncc(-c2ccc(NC(=O)C(CCO)c3csc(NS(=O)(=O)C4CC4)n3)cc2)n1]

The racemate T410 was separated by chiral preparative HPLC [Chiralpak® IB (Daicel Ltd.) column (4.6 mm×25 mm), flow rate 0.5 mL min⁻¹ eluting with a mixture of (30% of ethanol) ethanol in heptane+0.2% Et₂NH, UV detection at 254 nm followed by SCX (300 mg) purification (elution with MeOH) to afford:
2-(2-(cyclopropanesulfonamido)thiazol-4-yl)-N-(4-(6-ethoxypyrazin-2-yl)phenyl)-2-methoxyacetamide T410

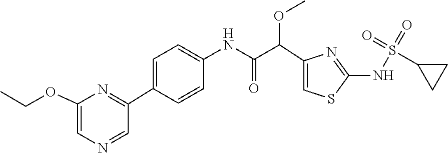 [SMILES: CCOc1cncc(-c2ccc(NC(=O)C(OC)c3csc(NS(=O)(=O)C4CC4)n3)cc2)n1]

The racemate T410 was prepared using Method 1b was separated by chiral preparative HPLC using a Diacel Chiralpak IB column (30% EtOH (0.1% DEA) in iso-hexane (0.2% DEA) to afford:

Peak 1: Stereochemistry of Product was Unassigned
2-(2-(cyclopropanesulfonamido)thiazol-4-yl)-N-(4-(6-ethoxypyrazin-2-yl)phenyl)-2-methoxyacetamide T330.

Peak 2: Stereochemistry of Product was Unassigned
2-(2-(cyclopropanesulfonamido)thiazol-4-yl)-N-(4-(6-ethoxypyrazin-2-yl)phenyl)-2-methoxyacetamide T331.

TABLE 7
Compounds T332-T416

| | Name/Structure |
|---|---|
| | (All examples containing chiral |
| T# | centres are racemates unless stated) |
| T332 | 2-(2-((2- |
| | methoxyethyl)sulfonamido)thiazol-4-yl)-2- |
| | methyl-N-(5-(6-(trifluoronnethyl)pyrazin-2- |
| | yl)pyridin-2-yl)propanamide |
| | 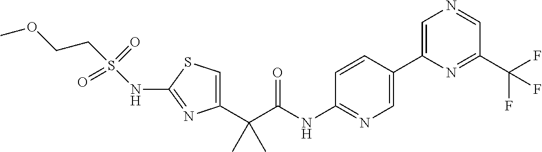 [SMILES: COCCS(=O)(=O)Nc1nc(C(C)(C)C(=O)Nc2ccc(-c3cncc(C(F)(F)F)n3)cn2)cs1] |
| T333 | 2-(2-(cyclopentanesulfonamido)thiazol-4- |
| | yl)-2-methyl-N-(4-(pyridin-3- |
| | yl)phenyl)propanamide |
| | 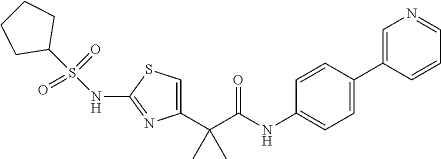 [SMILES: CC(C)(C(=O)Nc1ccc(-c2cccnc2)cc1)c1csc(NS(=O)(=O)C2CCCC2)n1] |
| T334 | 2-(2-(cyclopentanesulfonamido)thiazol-4- |
| | yl)-2-methyl-N-(4-(6- |
| | (trifluoromethyl)pyrazin-2- |
| | yl)phenyl)propanamide |
| | 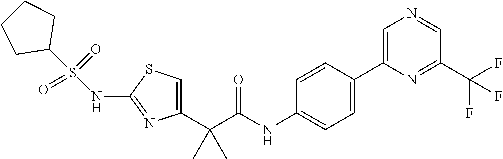 [SMILES: CC(C)(C(=O)Nc1ccc(-c2cncc(C(F)(F)F)n2)cc1)c1csc(NS(=O)(=O)C2CCCC2)n1] |
| T335 | 2-(2-(cyclopentanesulfonamido)thiazol-4- |
| | yl)-N-(4-(6-ethoxypyrazin-2-yl)phenyl)-2- |
| | methylpropanamide |
| | 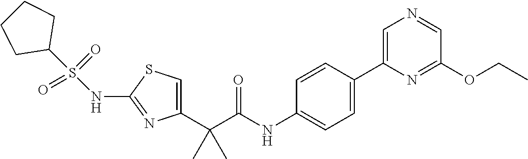 [SMILES: CCOc1cncc(-c2ccc(NC(=O)C(C)(C)c3csc(NS(=O)(=O)C4CCCC4)n3)cc2)n1] |
| T336 | 2-(2-(cyclopropanesulfonamido)thiazol-4- |
| | yl)-N-(5-(6-isopropylpyrazin-2-yl)pyridin- |
| | 2-yl)-2-methylpropanamide |
| | 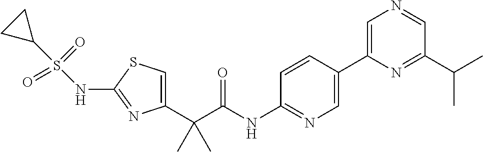 [SMILES: CC(C)c1cncc(-c2ccc(NC(=O)C(C)(C)c3csc(NS(=O)(=O)C4CC4)n3)nc2)n1] |
| T337 | 2-(2-(cyclopropanesulfonamido)thiazol-4- |
| | yl)-N-(5′-ethoxy-[3,3′-bipyridin]-6-yl)-2- |
| | methylpropanamide |
| | 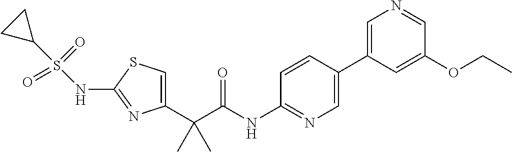 [SMILES: CCOc1cncc(-c2ccc(NC(=O)C(C)(C)c3csc(NS(=O)(=O)C4CC4)n3)nc2)c1] |
| T338 | 2-(2-(cyclopropanesulfonamido)thiazol-4- |
| | yl)-N-(5-(6-(2-hydroxypropan-2- |
| | yl)pyrazin-2-yl)pyridin-2-yl)-2- |
| | methylpropanamide |
| | 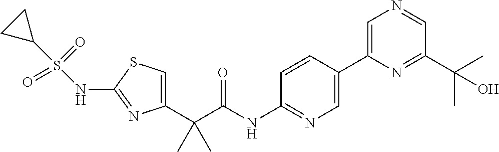 [SMILES: CC(C)(O)c1cncc(-c2ccc(NC(=O)C(C)(C)c3csc(NS(=O)(=O)C4CC4)n3)nc2)n1] |
| T339 | 2-(2-(cyclopropanesulfonamido)thiazol-4- |
| | yl)-N-(5-(6-(2-methoxypropan-2- |
| | yl)pyrazin-2-yl)pyridin-2-yl)-2- |
| | methylpropanamide |
| | 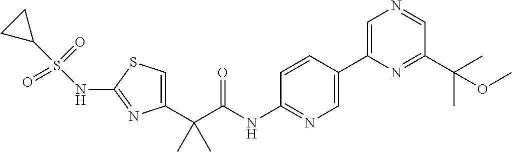 [SMILES: COC(C)(C)c1cncc(-c2ccc(NC(=O)C(C)(C)c3csc(NS(=O)(=O)C4CC4)n3)nc2)n1] |
| T340 | 2-(2-(cyclopropanesulfonamido)-5- |
| | methylthiazol-4-yl)-2-methyl-N-(5-(6- |
| | (trifluoromethyl)pyrazin-2-yl)pyridin-2- |
| | yl)propanamide |
| | 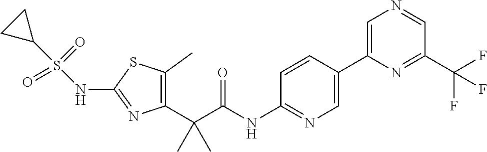 [SMILES: Cc1sc(NS(=O)(=O)C2CC2)nc1C(C)(C)C(=O)Nc1ccc(-c2cncc(C(F)(F)F)n2)cn1] |
| T341 | 2-(2-(cyclopropanesulfonamido)thiazol-4- |
| | yl)-N-(5-(6-ethoxypyrazin-2-yl)pyridin-2- |
| | yl)-2-methylpropanamide |

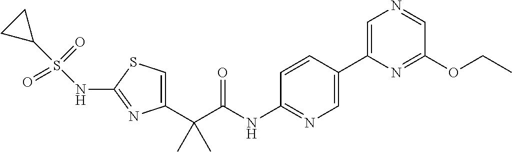 [SMILES: CCOc1cncc(-c2ccc(NC(=O)C(C)(C)c3csc(NS(=O)(=O)C4CC4)n3)nc2)n1]

| | |
|---|---|
| T342 | 2-(2-(cyclopropanesulfonamido)-5- |
| | methylthiazol-4-yl)-N-(5-(6- |
| | ethoxypyrazin-2-yl)pyridin-2-yl)-2- |
| | methylpropanamide |
| | 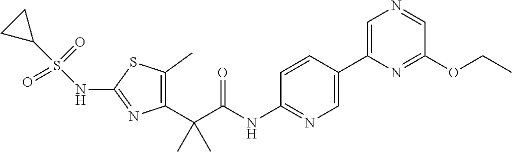 [SMILES: CCOc1cncc(-c2ccc(NC(=O)C(C)(C)c3nc(NS(=O)(=O)C4CC4)sc3C)nc2)n1] |
| T343 | N-(4-(5-chloropyridin-3-yl)-2- |
| | (trifluoromethyl)phenyl)-2-(2- |
| | (cyclopropanesulfonamido)thiazol-4-yl)-2- |
| | methylpropanamide |
| | 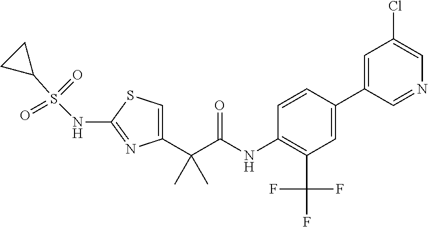 [SMILES: CC(C)(C(=O)Nc1ccc(-c2cncc(Cl)c2)cc1C(F)(F)F)c1csc(NS(=O)(=O)C2CC2)n1] |
| T344 | N-(4-(5-cyanopyridin-3-yl)-2- |
| | (trifluoromethyl)phenyl)-2-(2- |
| | (cyclopropanesulfonamido)thiazol-4-yl)-2- |
| | methylpropanamide |
| | 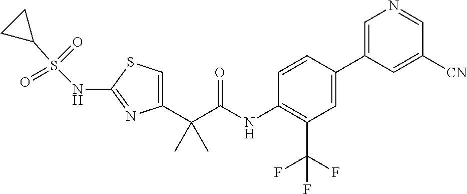 [SMILES: CC(C)(C(=O)Nc1ccc(-c2cncc(C#N)c2)cc1C(F)(F)F)c1csc(NS(=O)(=O)C2CC2)n1] |
| T345 | 2-(2-(cyclopropanesulfonamido)thiazol-4- |
| | yl)-N-(4-(5-fluoropyridin-3-yl)-2- |
| | (trifluoromethyl)phenyl)-2- |
| | methylpropanamide |
| | 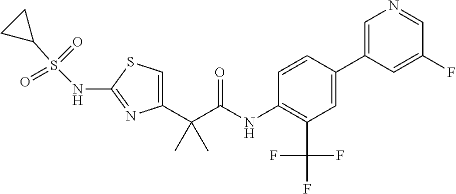 [SMILES: CC(C)(C(=O)Nc1ccc(-c2cncc(F)c2)cc1C(F)(F)F)c1csc(NS(=O)(=O)C2CC2)n1] |
| T346 | 2-(2-(cyclopropanesulfonamido)thiazol-4- |
| | yl)-2-methyl-N-(2-(trifluoromethyl)-4-(6- |
| | (trifluoromethyl)pyrazin-2- |
| | yl)phenyl)propanamide |
| | 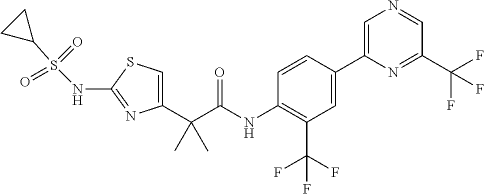 [SMILES: CC(C)(C(=O)Nc1ccc(-c2cncc(C(F)(F)F)n2)cc1C(F)(F)F)c1csc(NS(=O)(=O)C2CC2)n1] |
| T347 | 2-(2-(cyclopropanesulfonamido)thiazol-4- |
| | yl)-N-(4-(6-ethoxypyrazin-2-yl)-2- |
| | (trifluoromethyl)phenyl)-2- |
| | methylpropanamide |
| | 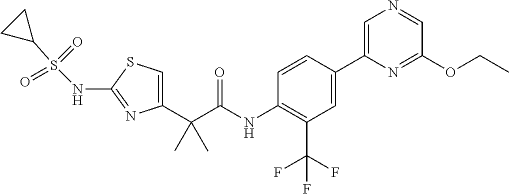 [SMILES: CCOc1cncc(-c2ccc(NC(=O)C(C)(C)c3csc(NS(=O)(=O)C4CC4)n3)c(C(F)(F)F)c2)n1] |
| T348 | N-(4-(5-chloropyridin-3-yl)-2,6- |
| | diethylphenyl)-2-(2- |
| | (cyclopropanesulfonamido)thiazol-4-yl)-2- |
| | methylpropanamide |
| | 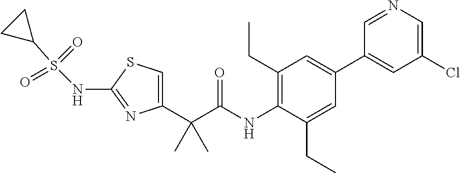 [SMILES: CCc1cc(-c2cncc(Cl)c2)cc(CC)c1NC(=O)C(C)(C)c1csc(NS(=O)(=O)C2CC2)n1] |
| T349 | N-(4-(5-cyanopyridin-3-yl)-2,6- |
| | diethylphenyl)-2-(2- |
| | (cyclopropanesulfonamido)thiazol-4-yl)-2- |
| | methylpropanamide |
| | 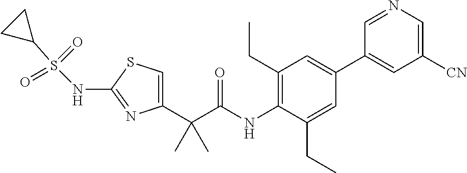 [SMILES: CCc1cc(-c2cncc(C#N)c2)cc(CC)c1NC(=O)C(C)(C)c1csc(NS(=O)(=O)C2CC2)n1] |
| T350 | 2-(2-(cyclopropanesulfonamido)-5- |
| | methylthiazol-4-yl)-N-(2-fluoro-4-(5- |
| | (trifluoromethyl)pyridin-3-yl)phenyl)-2- |
| | methylpropanamide |
| | 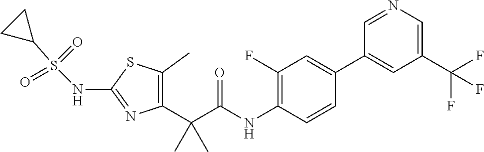 [SMILES: Cc1sc(NS(=O)(=O)C2CC2)nc1C(C)(C)C(=O)Nc1ccc(-c2cncc(C(F)(F)F)c2)cc1F] |
| T351 | N-(4-(5-chloropyridin-3-yl)-2,6- |
| | difluorophenyl)-2-(2- |
| | (cyclopropanesulfonamido)thiazol-4-yl)-2- |
| | methylpropanamide |
| | 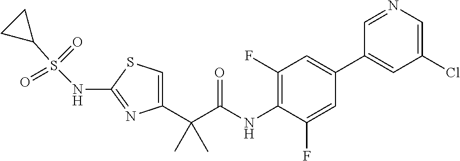 [SMILES: CC(C)(C(=O)Nc1c(F)cc(-c2cncc(Cl)c2)cc1F)c1csc(NS(=O)(=O)C2CC2)n1] |
| T352 | N-(4-(5-chloropyridin-3-yl)-2-fluoro-5- |
| | methylphenyl)-2-(2- |
| | (cyclopropanesulfonamido)thiazol-4-yl)- |
| | 2-methylpropanamide |
| | 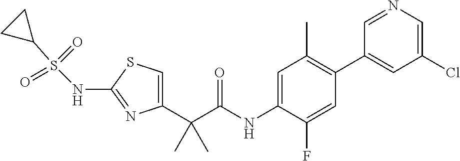 [SMILES: Cc1cc(NC(=O)C(C)(C)c2csc(NS(=O)(=O)C3CC3)n2)c(F)cc1-c1cncc(Cl)c1] |
| T353 | 2-(2-(cyclopropanesulfonamido)thiazol-4- |
| | yl)-N-(2-fluoro-4-(6-(2-methoxypropan-2- |
| | yl)pyrazin-2-yl)phenyl)-2- |
| | methylpropanamide |
| | 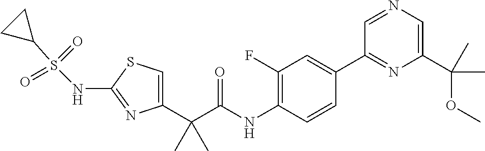 [SMILES: COC(C)(C)c1cncc(-c2ccc(NC(=O)C(C)(C)c3csc(NS(=O)(=O)C4CC4)n3)c(F)c2)n1] |
| T354 | 2-(2-(cyclopropanesulfonamido)-5- |
| | methylthiazol-4-yl)-N-(2-fluoro-4-(6- |
| | (trifluoromethyl)pyrazin-2-yl)phenyl)-2- |
| | methylpropanamide |
| | 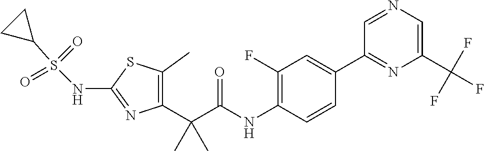 [SMILES: Cc1sc(NS(=O)(=O)C2CC2)nc1C(C)(C)C(=O)Nc1ccc(-c2cncc(C(F)(F)F)n2)cc1F] |
| T355 | N-(4-(6-cyanopyrazin-2-yl)-2- |
| | fluorophenyl)-2-(2- |
| | (cyclopropanesulfonamido)thiazol-4-yl)-2- |
| | methylpropanamide |
| | 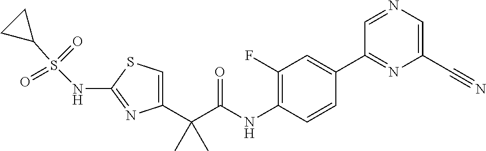 [SMILES: CC(C)(C(=O)Nc1ccc(-c2cncc(C#N)n2)cc1F)c1csc(NS(=O)(=O)C2CC2)n1] |
| T356 | 2-(2-(cyclopropanesulfonamido)thiazol-4- |
| | yl)-N-(4-(6-ethylpyrazin-2-yl)-2- |
| | fluorophenyl)-2-methylpropanamide |
| | 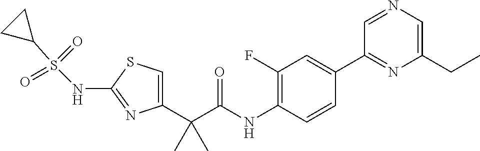 [SMILES: CCc1cncc(-c2ccc(NC(=O)C(C)(C)c3csc(NS(=O)(=O)C4CC4)n3)c(F)c2)n1] |
| T357 | 2-(2-(cyclopropanesulfonamido)thiazol-4- |
| | yl)-N-(2-fluoro-4-(6-isopropoxypyrazin-2- |
| | yl)phenyl)-2-methylpropanamide |
| | 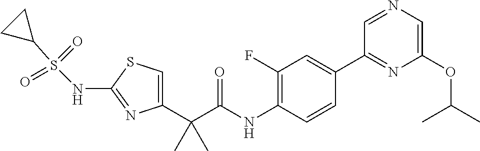 [SMILES: CC(C)Oc1cncc(-c2ccc(NC(=O)C(C)(C)c3csc(NS(=O)(=O)C4CC4)n3)c(F)c2)n1] |
| T358 | 2-(2-(cyclopropanesulfonamido)-5- |
| | methylthiazol-4-yl)-N-(4-(6- |
| | ethoxypyrazin-2-yl)-2-fluorophenyl)-2- |
| | methylpropanamide |
| | 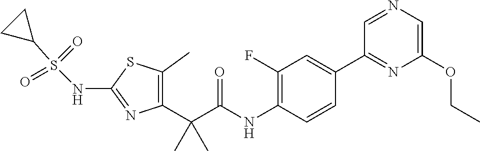 [SMILES: CCOc1cncc(-c2ccc(NC(=O)C(C)(C)c3nc(NS(=O)(=O)C4CC4)sc3C)c(F)c2)n1] |
| T359 | N-(4-(5-chloropyridin-3-yl)-2- |
| | isopropylphenyl)-2-(2- |
| | (cyclopropanesulfonamido)thiazol-4-yl)-2- |
| | methylpropanamide |
| | 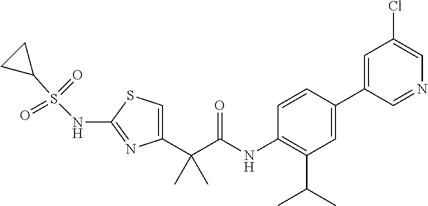 [SMILES: CC(C)c1cc(-c2cncc(Cl)c2)ccc1NC(=O)C(C)(C)c1csc(NS(=O)(=O)C2CC2)n1] |
| T360 | N-(4-(5-cyanopyridin-3-yl)-2- |
| | isopropylphenyl)-2-(2- |
| | (cyclopropanesulfonamido)thiazol-4-yl)-2- |
| | methylpropanamide |
| | 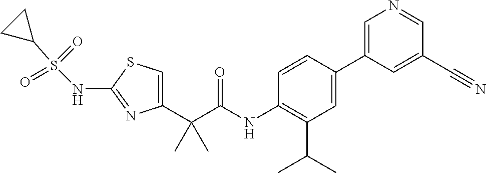 [SMILES: CC(C)c1cc(-c2cncc(C#N)c2)ccc1NC(=O)C(C)(C)c1csc(NS(=O)(=O)C2CC2)n1] |
| T361 | 2-(2-(cyclopropanesulfonamido)thiazol-4- |
| | yl)-N-(2-isopropyl-4-(6- |
| | (trifluoromethyl)pyrazin-2-yl)phenyl)-2- |
| | methylpropanamide |
| | 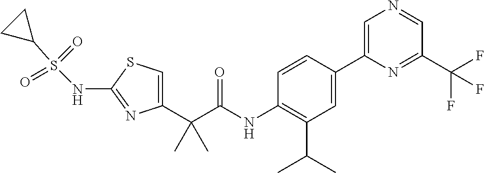 [SMILES: CC(C)c1cc(-c2cncc(C(F)(F)F)n2)ccc1NC(=O)C(C)(C)c1csc(NS(=O)(=O)C2CC2)n1] |
| T362 | 2-(2-(cyclopropanesulfonamido)thiazol-4- |
| | yl)-N-(4-(6-ethoxypyrazin-2-yl)-2- |
| | isopropylphenyl)-2-methylpropanamide |
| | 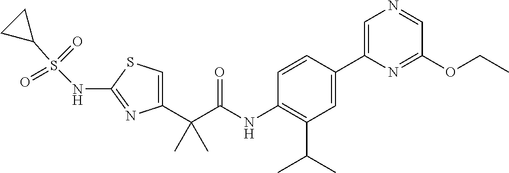 [SMILES: CCOc1cncc(-c2ccc(NC(=O)C(C)(C)c3csc(NS(=O)(=O)C4CC4)n3)c(C(C)C)c2)n1] |
| T363 | N-(4-(5-chloropyridin-3-yl)-3-fluoro-2- |
| | methylphenyl)-2-(2- |
| | (cyclopropanesulfonamido)thiazol-4-yl)- |
| | 2-methylpropanamide |
| | 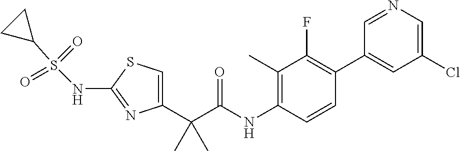 [SMILES: Cc1c(NC(=O)C(C)(C)c2csc(NS(=O)(=O)C3CC3)n2)ccc(-c2cncc(Cl)c2)c1F] |
| T364 | N-(4-(5-chloropyridin-3-yl)-5-fluoro-2- |
| | methylphenyl)-2-(2- |
| | (cyclopropanesulfonamido)thiazol-4-yl)- |
| | 2-methylpropanamide |
| | 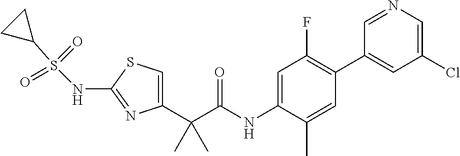 [SMILES: Cc1cc(-c2cncc(Cl)c2)c(F)cc1NC(=O)C(C)(C)c1csc(NS(=O)(=O)C2CC2)n1] |
| T365 | N-(4-(5-chloropyridin-3-yl)-2,3- |
| | dimethylphenyl)-2-(2- |
| | (cyclopropanesulfonamido)thiazol-4-yl)-2- |
| | methylpropanamide |
| | 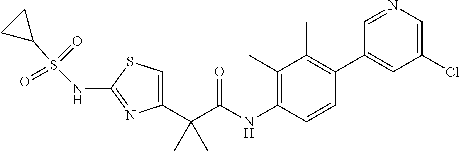 [SMILES: Cc1c(NC(=O)C(C)(C)c2csc(NS(=O)(=O)C3CC3)n2)ccc(-c2cncc(Cl)c2)c1C] |
| T366 | N-(4-(5-chloropyridin-3-yl)-2,5- |
| | dimethylphenyl)-2-(2- |
| | (cyclopropanesulfonamido)thiazol-4-yl)-2- |
| | methylpropanamide |
| | 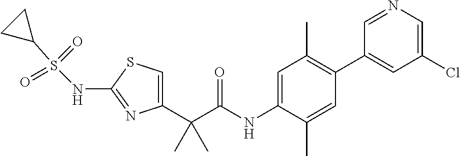 [SMILES: Cc1cc(-c2cncc(Cl)c2)c(C)cc1NC(=O)C(C)(C)c1csc(NS(=O)(=O)C2CC2)n1] |
| T367 | N-(4-(5-cyanopyridin-3-yl)-3-fluoro-2- |
| | methylphenyl)-2-(2- |
| | (cyclopropanesulfonamido)thiazol-4-yl)- |
| | 2-methylpropanamide |
| | 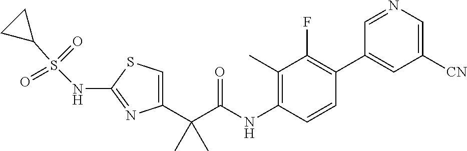 [SMILES: Cc1c(NC(=O)C(C)(C)c2csc(NS(=O)(=O)C3CC3)n2)ccc(-c2cncc(C#N)c2)c1F] |
| T368 | 2-(2-(cyclopropanesulfonamido)thiazol-4- |
| | yl)-2-methyl-N-(2-methyl-4-(6- |
| | (trifluoromethyl) pyrazin-2- |
| | yl)phenyl)propanamide |
| | 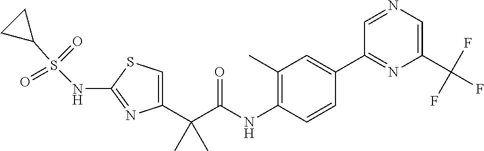 [SMILES: Cc1cc(-c2cncc(C(F)(F)F)n2)ccc1NC(=O)C(C)(C)c1csc(NS(=O)(=O)C2CC2)n1] |
| T369 | N-(4-(5-chloropyridin-3-yl)-5-fluoro-2- |
| | methoxyphenyl)-2-(2- |
| | (cyclopropanesulfonamido)thiazol-4-yl)- |
| | 2-methylpropanamide |
| | 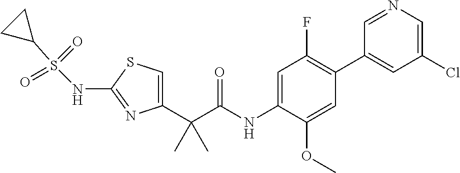 [SMILES: COc1cc(-c2cncc(Cl)c2)c(F)cc1NC(=O)C(C)(C)c1csc(NS(=O)(=O)C2CC2)n1] |
| T370 | N-(4-(5-chloropyridin-3-yl)-3- |
| | (trifluoromethyl)phenyl)-2-(2- |
| | (cyclopropanesulfonamido)thiazol-4-yl)-2- |
| | methylpropanamide |
| | 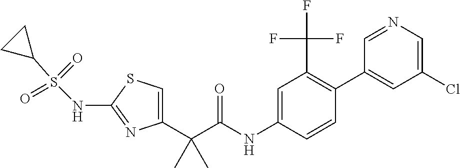 [SMILES: CC(C)(C(=O)Nc1ccc(-c2cncc(Cl)c2)c(C(F)(F)F)c1)c1csc(NS(=O)(=O)C2CC2)n1] |
| T371 | 2-(2-(cyclopropanesulfonamido)thiazol-4- |
| | yl)-N-(4-(6-ethoxypyrazin-2-yl)-3- |
| | methylphenyl)-2-methylpropanamide |
| | 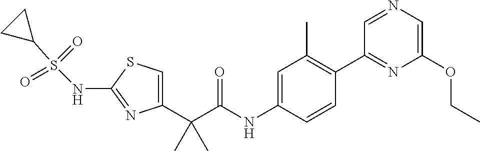 [SMILES: CCOc1cncc(-c2ccc(NC(=O)C(C)(C)c3csc(NS(=O)(=O)C4CC4)n3)cc2C)n1] |
| T372 | N-(4-(5-chloropyridin-3-yl)-3- |
| | ethoxyphenyl)-2-(2- |
| | (cyclopropanesulfonamido)thiazol-4-yl)-2- |
| | methylpropanamide |
| | 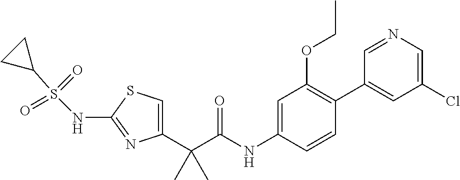 [SMILES: CCOc1cc(NC(=O)C(C)(C)c2csc(NS(=O)(=O)C3CC3)n2)ccc1-c1cncc(Cl)c1] |
| T373 | N-(4-(5-chloropyridin-3-yl)phenyl)-1-(2- |
| | (cyclopropanesulfonamido)thiazol-4- |
| | yl)cyclopropane-1-carboxamide |
| | 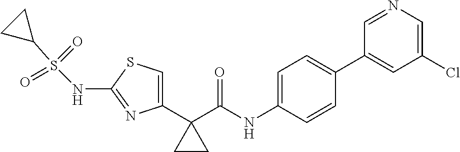 [SMILES: O=C(Nc1ccc(-c2cncc(Cl)c2)cc1)C1(c2csc(NS(=O)(=O)C3CC3)n2)CC1] |
| T374 | N-(4-(5-cyanopyridin-3-yl)phenyl)-2-(2- |
| | (cyclopropanesulfonamido)-5- |
| | methylthiazol-4-yl)-2-methylpropanamide |
| | 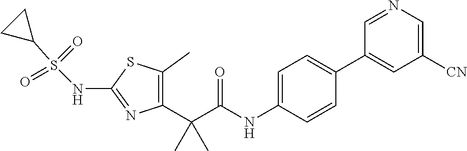 [SMILES: Cc1sc(NS(=O)(=O)C2CC2)nc1C(C)(C)C(=O)Nc1ccc(-c2cncc(C#N)c2)cc1] |
| T375 | 2-(2-(cyclopropanesulfonamido)thiazol-4- |
| | yl)-N-(4-(6-(2-methoxypropan-2- |
| | yl)pyrazin-2-yl)phenyl)-2- |
| | methylpropanamide |
| | 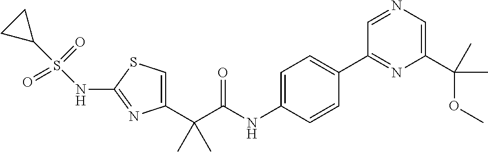 [SMILES: COC(C)(C)c1cncc(-c2ccc(NC(=O)C(C)(C)c3csc(NS(=O)(=O)C4CC4)n3)cc2)n1] |
| T376 | 2-(5-chloro-2- |
| | (cyclopropanesulfonamido)thiazol-4-yl)- |
| | N-(4-(6-ethoxypyrazin-2-yl)phenyl)-2- |
| | methylpropanamide |
| | 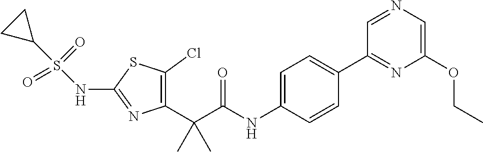 [SMILES: CCOc1cncc(-c2ccc(NC(=O)C(C)(C)c3nc(NS(=O)(=O)C4CC4)sc3Cl)cc2)n1] |
| T377 | 2-(2-(cyclopropanesulfonamido)-5- |
| | methoxythiazol-4-yl)-N-(4-(6- |
| | ethoxypyrazin-2-yl)phenyl)-2- |
| | methylpropanamide |
| | 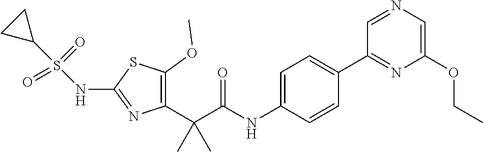 [SMILES: CCOc1cncc(-c2ccc(NC(=O)C(C)(C)c3nc(NS(=O)(=O)C4CC4)sc3OC)cc2)n1] |
| T378 | N-(4-(6-(cyclopentylmethoxy)pyrazin-2- |
| | yl)phenyl)-2-(2- |
| | (cyclopropanesulfonamido)thiazol-4-yl)- |
| | 2-methylpropanamide |
| | 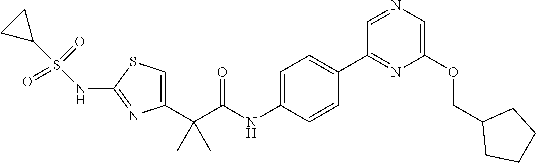 [SMILES: CC(C)(C(=O)Nc1ccc(-c2cncc(OCC3CCCC3)n2)cc1)c1csc(NS(=O)(=O)C2CC2)n1] |
| T379 | 2-(2-(cyclopropanesulfonamido)thiazol-4- |
| | yl)-N-(4-(6-hydroxypyrazin-2-yl)phenyl)-2- |
| | methylpropanamide |
| | 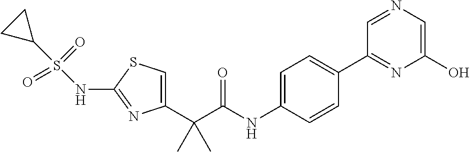 [SMILES: CC(C)(C(=O)Nc1ccc(-c2cncc(O)n2)cc1)c1csc(NS(=O)(=O)C2CC2)n1] |
| T380 | 2-(2-(ethylsulfonamido)thiazol-4-yl)-2- |
| | methyl-N-(5′-(trifluoromethyl)-[3,3′- |
| | bipyridin]-6-yl)propanamide |
| | 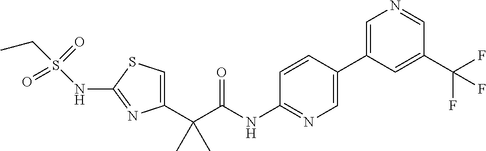 [SMILES: CCS(=O)(=O)Nc1nc(C(C)(C)C(=O)Nc2ccc(-c3cncc(C(F)(F)F)c3)cn2)cs1] |
| T381 | 2-(2-(ethylsulfonamido)thiazol-4-yl)-2- |
| | methyl-N-(5-(6-(trifluoromethyl)pyrazin-2- |
| | yl)pyridin-2-yl)propanamide |
| | 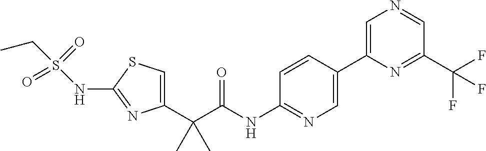 [SMILES: CCS(=O)(=O)Nc1nc(C(C)(C)C(=O)Nc2ccc(-c3cncc(C(F)(F)F)n3)cn2)cs1] |
| T382 | N-(5-(6-ethoxypyrazin-2-yl)pyridin-2-yl)-2- |
| | (2-(ethylsulfonamido)thiazol-4-yl)-2- |
| | methylpropanamide |
| | 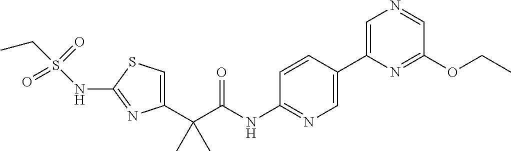 [SMILES: CCOc1cncc(-c2ccc(NC(=O)C(C)(C)c3csc(NS(=O)(=O)CC)n3)nc2)n1] |
| T383 | 2-(2-(ethylsulfonamido)thiazol-4-yl)-N-(2- |
| | fluoro-4-(6-isopropoxypyrazin-2- |
| | yl)phenyl)-2-methylpropanamide |
| | 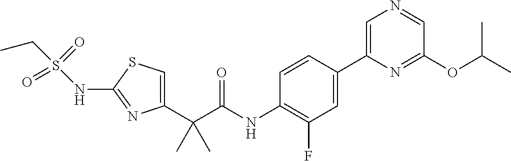 [SMILES: CCS(=O)(=O)Nc1nc(C(C)(C)C(=O)Nc2ccc(-c3cncc(OC(C)C)n3)cc2F)cs1] |
| T384 | N-(4-(5-cyanopyridin-3-yl)phenyl)-2-(2- |
| | (ethylsulfonamido)thiazol-4-yl)-2- |
| | methylpropanamide |
| | 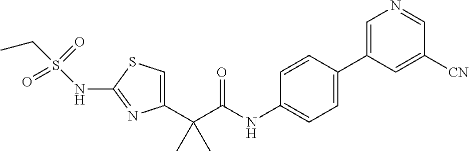 [SMILES: CCS(=O)(=O)Nc1nc(C(C)(C)C(=O)Nc2ccc(-c3cncc(C#N)c3)cc2)cs1] |
| T385 | 2-(2-(ethylsulfonamido)thiazol-4-yl)-N-(4- |
| | (5-fluoropyridin-3-yl)phenyl)-2- |
| | methylpropanamide |
| | 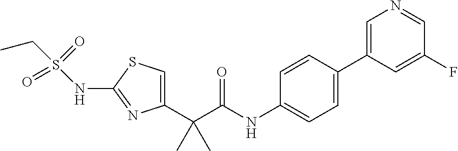 [SMILES: CCS(=O)(=O)Nc1nc(C(C)(C)C(=O)Nc2ccc(-c3cncc(F)c3)cc2)cs1] |
| T386 | 2-(2-(ethylsulfonamido)thiazol-4-yl)-2- |
| | methyl-N-(4-(pyridin-3- |
| | yl)phenyl)propanamide |
| | 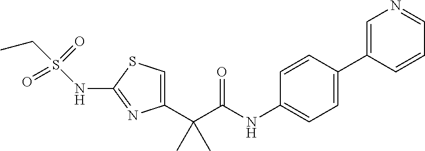 [SMILES: CCS(=O)(=O)Nc1nc(C(C)(C)C(=O)Nc2ccc(-c3cccnc3)cc2)cs1] |
| T387 | 2-(2-(ethylsulfonamido)thiazol-4-yl)-2- |
| | methyl-N-(4-(6-(trifluoromethyppyrazin-2- |
| | yl)phenyl)propanamide |
| | 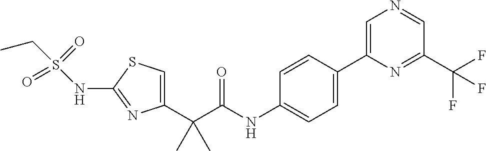 [SMILES: CCS(=O)(=O)Nc1nc(C(C)(C)C(=O)Nc2ccc(-c3cncc(C(F)(F)F)n3)cc2)cs1] |
| T388 | 2-(2-(ethylsulfonamido)thiazol-4-yl)-N-(4- |
| | (6-isopropoxypyrazin-2-yl)phenyl)-2- |
| | methylpropanamide |
| | 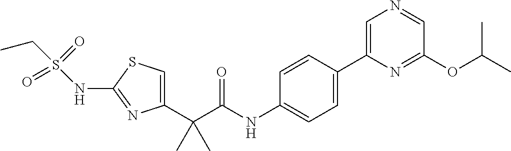 [SMILES: CCS(=O)(=O)Nc1nc(C(C)(C)C(=O)Nc2ccc(-c3cncc(OC(C)C)n3)cc2)cs1] |
| T389 | 2-methyl-2-(2- |
| | (methylsulfonamido)thiazol-4-yl)-N-(5′- |
| | (trifluoromethyl)-[3,3′-bipyridin]-6- |
| | yl)propanamide |
| | 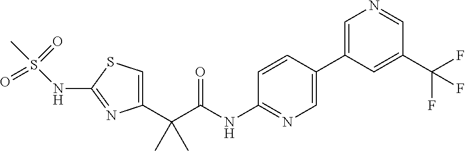 [SMILES: CC(C)(C(=O)Nc1ccc(-c2cncc(C(F)(F)F)c2)cn1)c1csc(NS(C)(=O)=O)n1] |
| T390 | N-(5-(6-ethoxypyrazin-2-yl)pyridin-2-y0-2- |
| | methyl-2-(2-(methylsulfonamido)thiazol- |
| | 4-yl)propanamide |
| | 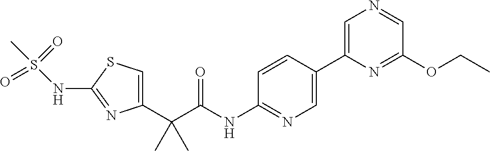 [SMILES: CCOc1cncc(-c2ccc(NC(=O)C(C)(C)c3csc(NS(C)(=O)=O)n3)nc2)n1] |
| T391 | N-(2-fluoro-4-(6-(trifluoromethyl)pyrazin- |
| | 2-yl)phenyl)-2-methyl-2-(2- |
| | (methylsulfonamido)thiazol-4- |
| | yl)propanamide |
| | 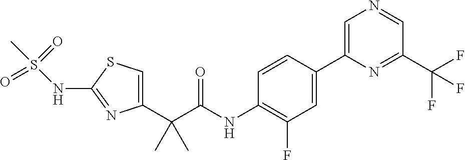 [SMILES: CC(C)(C(=O)Nc1ccc(-c2cncc(C(F)(F)F)n2)cc1F)c1csc(NS(C)(=O)=O)n1] |
| T392 | N-(2-fluoro-4-(6-isopropoxypyrazin-2- |
| | yl)phenyl)-2-methyl-2-(2- |
| | (methylsulfonamido)thiazol-4- |
| | yl)propanamide |
| | 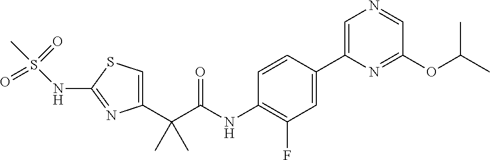 [SMILES: CC(C)Oc1cncc(-c2ccc(NC(=O)C(C)(C)c3csc(NS(C)(=O)=O)n3)c(F)c2)n1] |
| T393 | N-(4-(5-chloropyridin-3-yl)phenyl)-2- |
| | methyl-2-(2-(methylsulfonamido)thiazol- |
| | 4-yl)propanamide |
| | 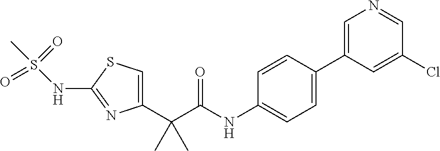 [SMILES: CC(C)(C(=O)Nc1ccc(-c2cncc(Cl)c2)cc1)c1csc(NS(C)(=O)=O)n1] |
| T394 | 2-methyl-2-(2- |
| | (methylsulfonamido)thiazol-4-yl)-N-(4-(6- |
| | (trifluoromethyl)pyrazin-2-yl)phenyl) |
| | propanamide |
| | 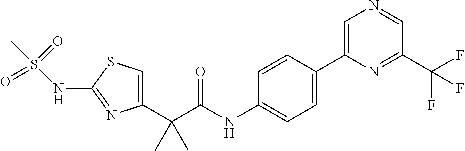 [SMILES: CC(C)(C(=O)Nc1ccc(-c2cncc(C(F)(F)F)n2)cc1)c1csc(NS(C)(=O)=O)n1] |
| T395 | N-(4-(6-isopropoxypyrazin-2-yl)phenyl)-2- |
| | methyl-2-(2-(methylsulfonamido)thiazol- |
| | 4-yl)propanamide |
| | 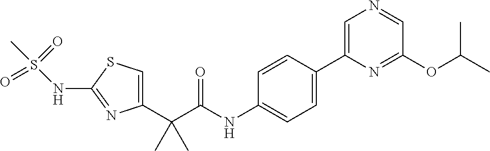 [SMILES: CC(C)Oc1cncc(-c2ccc(NC(=O)C(C)(C)c3csc(NS(C)(=O)=O)n3)cc2)n1] |
| T396 | 2-(2- |
| | ((cyclopropylmethyl)sulfonamido)thiazol- |
| | 4-yl)-2-methyl-N-(5-(6- |
| | (trifluoromethyl)pyrazin-2-yl)pyridin-2- |
| | yl)propanamide |
| | 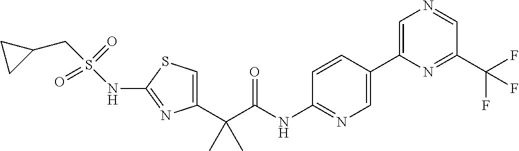 [SMILES: CC(C)(C(=O)Nc1ccc(-c2cncc(C(F)(F)F)n2)cn1)c1csc(NS(=O)(=O)CC2CC2)n1] |
| T397 | 1-(2-(cyclopropanesulfonamido)thiazol-4- |
| | yl)-N-(4-(6-(trifluoromethyl)pyrazin-2- |
| | yl)phenyl)cyclopropane-1-carboxamide |
| | 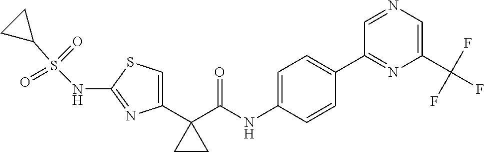 [SMILES: O=C(Nc1ccc(-c2cncc(C(F)(F)F)n2)cc1)C1(c2csc(NS(=O)(=O)C3CC3)n2)CC1] |
| T398 | 1-(2-(cyclopropanesulfonamido)thiazol-4- |
| | yl)-N-(4-(6-ethoxypyrazin-2- |
| | yl)phenyl)cyclopropane-1-carboxamide |
| | 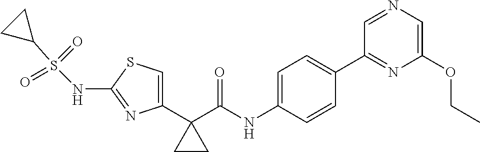 [SMILES: CCOc1cncc(-c2ccc(NC(=O)C3(c4csc(NS(=O)(=O)C5CC5)n4)CC3)cc2)n1] |
| T399 | 2-(2-(cyclopropanesulfonamido)thiazol-4- |
| | yl)-N-(2-fluoro-4-(6- |
| | (trifluoromethyppyrazin-2-yl)phenyl)-4- |
| | methylpropanamide |
| | 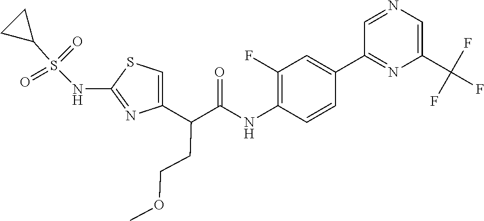 [SMILES: COCCC(C(=O)Nc1ccc(-c2cncc(C(F)(F)F)n2)cc1F)c1csc(NS(=O)(=O)C2CC2)n1] |
| T400 | 2-(2-(cyclopropanesulfonamido)thiazol-4- |
| | yl)-N-(2-fluoro-4-(6-isopropoxypyrazin-2- |
| | yl)phenyl)-4-methoxybutanamide |
| | 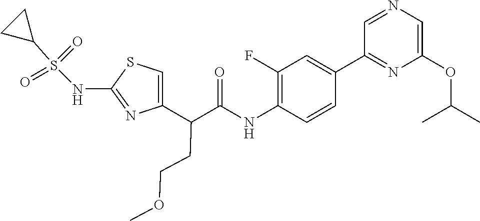 [SMILES: COCCC(C(=O)Nc1ccc(-c2cncc(OC(C)C)n2)cc1F)c1csc(NS(=O)(=O)C2CC2)n1] |
| T401 | 2-(2-(cyclopropanesulfonamido)thiazol-4- |
| | yl)-N-(5-(6-isopropylpyrazin-2-yl)pyridin- |
| | 2-yl)butanamide |
| | 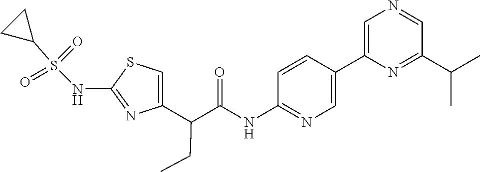 [SMILES: CCC(C(=O)Nc1ccc(-c2cncc(C(C)C)n2)cn1)c1csc(NS(=O)(=O)C2CC2)n1] |
| T402 | 2-(2-(cyclopropanesulfonamido)thiazol-4- |
| | yl)-N-(5-(6-(2-methoxypropan-2- |
| | yl)pyrazin-2-yl)pyridin-2-yl)butanamide |
| | 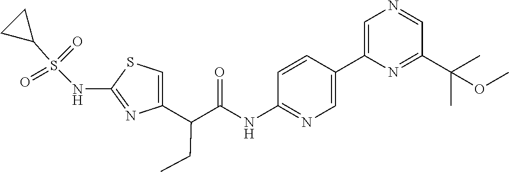 [SMILES: CCC(C(=O)Nc1ccc(-c2cncc(C(C)(C)OC)n2)cn1)c1csc(NS(=O)(=O)C2CC2)n1] |
| T403 | 2-(2-(cyclopropanesulfonamido)thiazol-4- |
| | yl)-N-(2-fluoro-4-(6-(2-methoxypropan-2- |
| | yl)pyrazin-2-yl)phenyl)butanamide |
| | 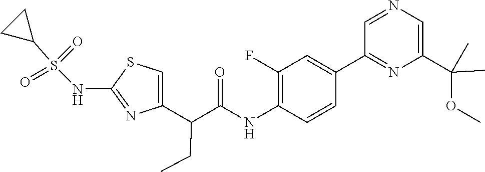 [SMILES: CCC(C(=O)Nc1ccc(-c2cncc(C(C)(C)OC)n2)cc1F)c1csc(NS(=O)(=O)C2CC2)n1] |
| T404 | N-(4-(6-cyanopyrazin-2-yl)-2- |
| | fluorophenyl)-2-(2- |
| | (cyclopropanesulfonamido)thiazol-4- |
| | yl)butanamide |
| | 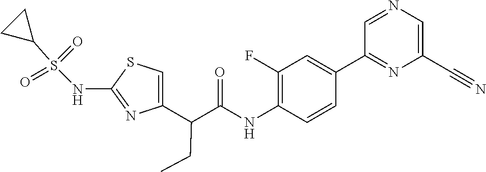 [SMILES: CCC(C(=O)Nc1ccc(-c2cncc(C#N)n2)cc1F)c1csc(NS(=O)(=O)C2CC2)n1] |
| T405 | 2-(2-(cyclopropanesulfonamido)thiazol-4- |
| | yl)-N-(4-(6-ethylpyrazin-2-yl)-2- |
| | fluorophenyl)butanamide |
| | 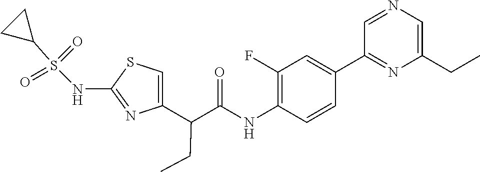 [SMILES: CCc1cncc(-c2ccc(NC(=O)C(CC)c3csc(NS(=O)(=O)C4CC4)n3)c(F)c2)n1] |
| T406 | 2-(2-(cyclopropanesulfonamido)thiazol-4- |
| | yl)-N-(4-(6-(2-methoxypropan-2- |
| | yl)pyrazin-2-yl)phenyl)butanamide |
| | 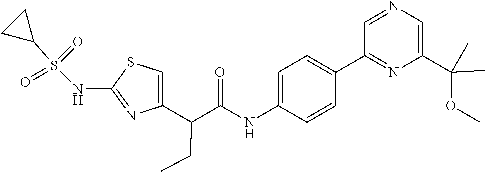 [SMILES: CCC(C(=O)Nc1ccc(-c2cncc(C(C)(C)OC)n2)cc1)c1csc(NS(=O)(=O)C2CC2)n1] |
| T407 | tert-butyl (1-(2- |
| | (cyclopropanesulfonamido)thiazol-4-yl)-2- |
| | ((4-(6-ethoxypyrazin-2-yl)phenyl)amino)- |
| | 2-oxoethyl)carbamate |
| | 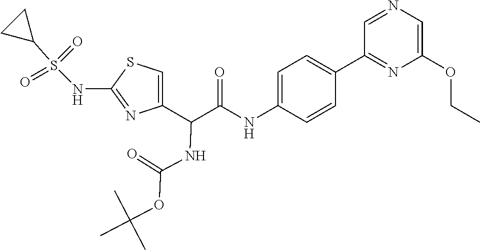 [SMILES: CCOc1cncc(-c2ccc(NC(=O)C(NC(=O)OC(C)(C)C)c3csc(NS(=O)(=O)C4CC4)n3)cc2)n1] |
| T408 | 2-(2-(cyclopropanesulfonamido)thiazol-4- |
| | yl)-N-(2-fluoro-4-(6- |
| | (trifluoromethyl)pyrazin-2-yl)phenyl)-2- |
| | methoxyacetamide |
| | 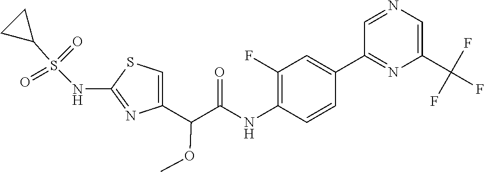 [SMILES: COC(C(=O)Nc1ccc(-c2cncc(C(F)(F)F)n2)cc1F)c1csc(NS(=O)(=O)C2CC2)n1] |
| T409 | 2-(2-(cyclopropanesulfonamido)thiazol-4- |
| | yl)-N-(4-(6-ethoxypyrazin-2-yl)-2- |
| | fluorophenyl)-2-methoxyacetamide |
| | 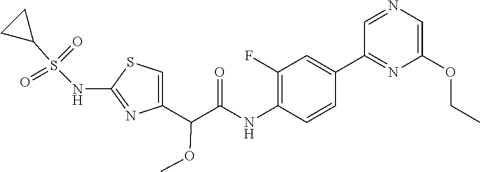 [SMILES: CCOc1cncc(-c2ccc(NC(=O)C(OC)c3csc(NS(=O)(=O)C4CC4)n3)c(F)c2)n1] |
| T410 | 2-(2-(cyclopropanesulfonamido)thiazol-4- |
| | yl)-N-(4-(6-ethoxypyrazin-2-yl)phenyl)-2- |
| | methoxyacetamide |
| | 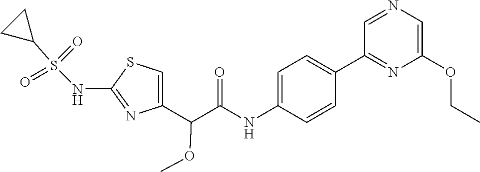 [SMILES: CCOc1cncc(-c2ccc(NC(=O)C(OC)c3csc(NS(=O)(=O)C4CC4)n3)cc2)n1] |
| T411 | 2-(2-(cyclopropanesulfonamido)thiazol-4- |
| | yl)-N-(4-(6-isopropoxypyrazin-2- |
| | yl)phenyl)-2-methoxyacetamide |
| | 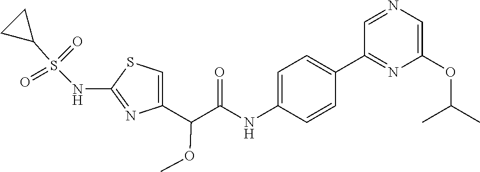 [SMILES: COC(C(=O)Nc1ccc(-c2cncc(OC(C)C)n2)cc1)c1csc(NS(=O)(=O)C2CC2)n1] |
| T412 | Single enantiomer - stereochemistry |
| | unassigned |
| | 2-(2-(cyclopropanesulfonamido)thiazol-4- |
| | yl)-N-(4-(6-ethoxypyrazin-2-yl)-2- |
| | fluorophenyl)butanamide |
| | 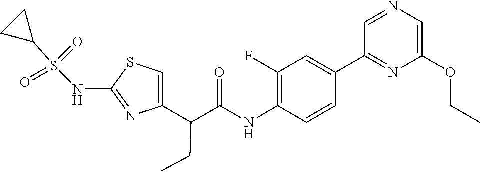 [SMILES: CCOc1cncc(-c2ccc(NC(=O)C(CC)c3csc(NS(=O)(=O)C4CC4)n3)c(F)c2)n1] |
| T413 | Single enantiomer - stereochemistry |
| | unassigned |
| | 2-(2-(cyclopropanesulfonamido)thiazol- |
| | 4-yl)-N-(4-(6-ethoxypyrazin-2-yl)-2- |
| | fluorophenyl)butanamide |
| | 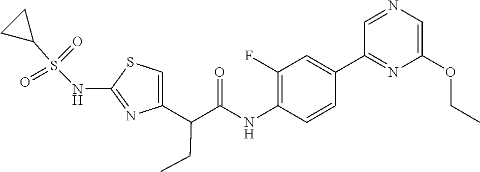 [SMILES: CCOc1cncc(-c2ccc(NC(=O)C(CC)c3csc(NS(=O)(=O)C4CC4)n3)c(F)c2)n1] |
| T414 | Single enantiomer - stereochemistry |
| | unassigned |
| | 2-(2-(cyclopropanesulfonamido)thiazol- |
| | 4-yl)-N-(5-(6-(trifluoromethyl)pyrazin-2- |
| | yl)pyridin-2-yl)butanamide |
| | 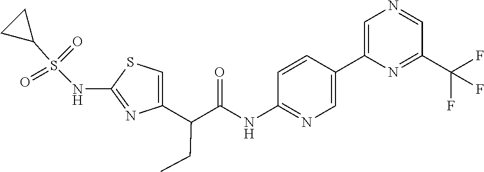 [SMILES: CCC(C(=O)Nc1ccc(-c2cncc(C(F)(F)F)n2)cn1)c1csc(NS(=O)(=O)C2CC2)n1] |
| T415 | Single enantiomer - stereochemistry |
| | unassigned |
| | 2-(2-(cyclopropanesulfonamido)thiazol-4- |
| | yl)-N-(2-fluoro-4-(5- |
| | (trifluoromethyl)pyridin-3- |
| | yl)phenyl)butanamide |

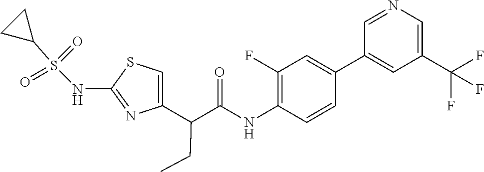 [SMILES: CCC(C(=O)Nc1ccc(-c2cncc(C(F)(F)F)c2)cc1F)c1csc(NS(=O)(=O)C2CC2)n1]

| | |
|---|---|
| T416 | Single enantiomer - stereochemistry |
| | unassigned |
| | 2-(2-(cyclopropanesulfonamido)thiazol-4- |
| | yl)-N-(2-fluoro-4-(5- |
| | (trifluoromethyl)pyridin-3- |
| | yl)phenyl)butanamide |
| | 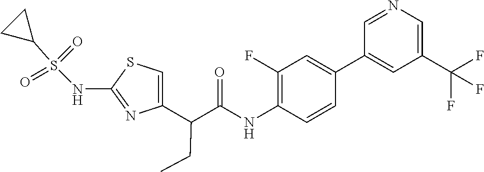 [SMILES: CCC(C(=O)Nc1ccc(-c2cncc(C(F)(F)F)c2)cc1F)c1csc(NS(=O)(=O)C2CC2)n1] |

2-(2-(Cyclopropanesulfonamido)thiazol-4-yl)-N-(4-(6-(ethylamino)pyrazin-2-yl)phenyl)butanamide T444

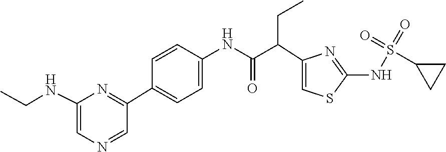 [SMILES: CCNc1cncc(-c2ccc(NC(=O)C(CC)c3csc(NS(=O)(=O)C4CC4)n3)cc2)n1]

2-Amino-2-(2-(cyclopropanesulfonamido)thiazolyl)-N-(5-(6-(trifluoromethyl)pyrazin-2-yl)pyridin-2-yl)acetamide hydrochloride T417

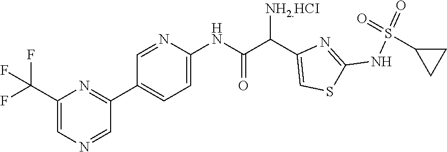

2-Amino-2-(2-(cyclopropanesulfonamido)thiazol-4-yl)-N-(4-(6-ethoxypyrazin-2-yl)-2-fluorophenyl)acetamide hydrochloride T419

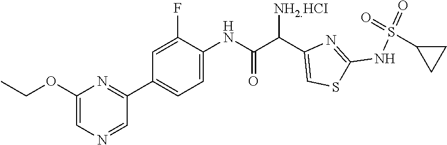

2-Amino-2-(2-(cyclopropanesulifonamido)thiazol-4-yl)-N-(2-fluoro-4-(6-(trifluoromethyl)pyrazin-2-yl)phenyl)acetamide T418

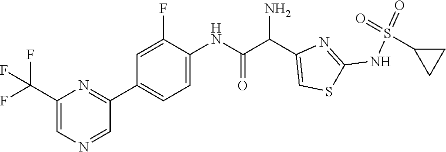 [SMILES: NC(C(=O)Nc1ccc(-c2cncc(C(F)(F)F)n2)cc1F)c1csc(NS(=O)(=O)C2CC2)n1]

2-(2-(Cyclopropanesulfonamido)thiazol-4-yl)-2-(dimethylamino)-N-(2-fluoro-4-(6-(trifluoromethyl)pyrazin-2-yl)phenyl)acetamide T420

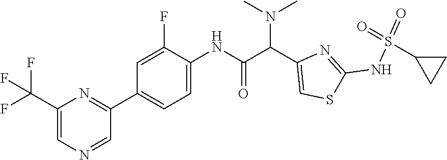 [SMILES: CN(C)C(C(=O)Nc1ccc(-c2cncc(C(F)(F)F)n2)cc1F)c1csc(NS(=O)(=O)C2CC2)n1]

2-(2-(Cyclopropanesulfonamido)thiazol-4-yl)-2-(dimethylamino)-N-(4-(6-ethoxypyrazin-2-yl)-2-fluorophenyl)acetamide T421

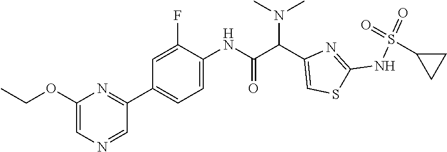 [SMILES: CCOc1cncc(-c2ccc(NC(=O)C(c3csc(NS(=O)(=O)C4CC4)n3)N(C)C)c(F)c2)n1]

N-(4-(1-((2-fluoro-4-(pyridin-3-yl)phenyl)amino)-2-methylpropan-2-yl)thiazol-2-yl)cyclopropanesulfonamide T466

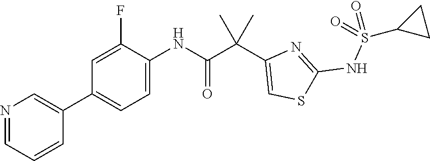 [SMILES: CC(C)(C(=O)Nc1ccc(-c2cccnc2)cc1F)c1csc(NS(=O)(=O)C2CC2)n1]

LiAlH₄ (0.870 mL, 1.74 mmol) was added into a solution of 2-(2-(cydopropanesulfonamido)thiazol-4-yl)-N-(2-fluoro-4-(pyridin-3-yl)phenyl)-2-methylpropanamide T42 (200 mg, 0.434 mmol) in THF (20 mL, 0.434 mmol) and the resulting solution was stirred at RT for 16 hrs. The reaction mixture was quenched with sat. NH₄Cl (aq, 50 mL) and extracted with EtOAc (3×50 mL). The organic extract was dried (MgSO₄), filtered and solvent removed in vacuo. The crude product was purified by chromatography on RP Flash C18 (12 g cartridge, 5-100% MeCN/10 mM ammonium bicarbonate) to afford N-(4-(1-((2-fluoro-4-(pyridin-3-yl)phenyl)amino)-2-methylpropan-2-yl)thiazol-2-yl)cyclopropanesulfonamide (23 mg, 0.050 mmol, 12% yield) as a white solid. Rt 1.83 min (HPLC basic); m/z 447 (M+H)⁺ (ES⁺). ¹H NMR (500 MHz, DMSO-d6) δ 12.57 (s, 1H), 8.82 (d, J=2.4 Hz, 1H), 8.47-8.43 (m, 1H), 8.00-7.95 (m, 1H), 7.46 (dd, J=13.4, 2.2 Hz, 1H), 7.39 (dd, J=8.0, 4.7 Hz, 1H), 7.35-7.31 (m, 1H), 6.92 (t, J=8.9 Hz, 1H), 6.40 (s, 1H), 5.50 (t, J=4.4 Hz, 1H), 3.36-3.28 (m, 2H (obscured by water peak)), 2.56-2.51 (m, 1H, (obscured by DMSO peak)), 1.27 (s, 6H), 0.90-0.79 (m, 4H).

Compounds of Formula (I-c)—Intermediates

Known synthetic intermediates were procured from commercial sources or were obtained using published literature procedures. Additional intermediates were prepared by the representative synthetic processes described herein.

The synthesis of INTE1 to INTE39 and INTF1 to INTF53 is disclosed in WO2019/106146 and uses the general methods disclosed below.

Method A: Reductive Amination

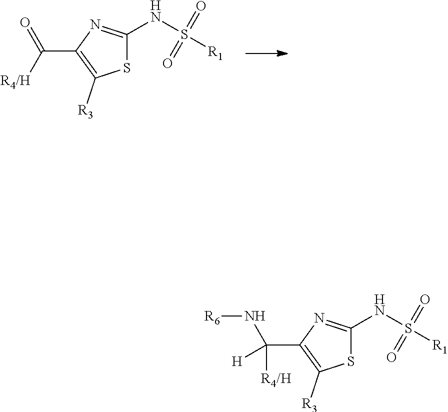

A solution of aldehyde/ketone (1 eq.) in THF was treated with AcOH (1 eq.), amine (1 eq.) and a reducing agent such as STAB (1.2 eq.) and stirred at RT for 1 hr. The reaction mixture was quenched by addition of MeOH then loaded directly on to SCX (1 g/mmol of substrate), washed with MeOH and the product was eluted with 1 M NH₃ in MeOH. The crude product was then concentrated onto silica and purified by normal phase chromatography.

Method B: Benzylamine Deprotection (TFA)

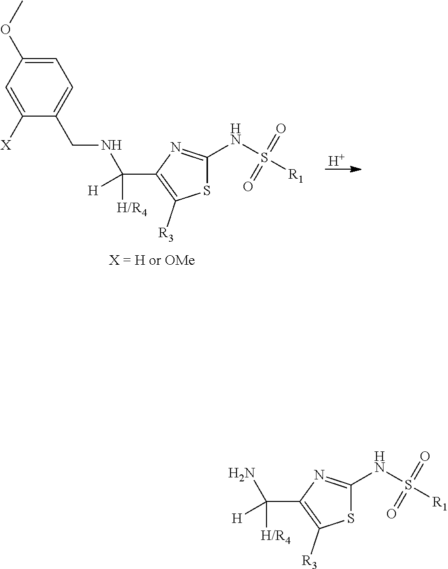

Benzylamine derivative (1 eq.) was dissolved in TFA (50 eq.) and heated to 70° C. for 1-24 hrs. The reaction was allowed to cool to RT, then was loaded on to SCX (1 g/mmol of substrate) and washed with MeOH. The required compound was eluted with 1% NH₃ in MeOH.

Method D: Ester Deprotection with TFA

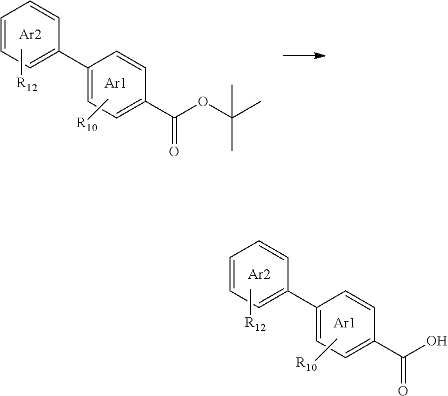

A solution of the ester (1 eq) in DCM (20 volumes) was treated with TFA (10 eq.) and stirred at RT for 3 hrs. The reaction mixture was then concentrated and azeotroped with MeOH and MeCN. No further purification was undertaken.

Method E: Ester Deprotection with Base

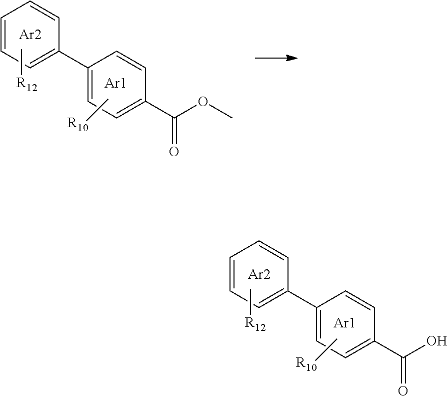

A solution of the ester (1 eq) in a mixture of THF/MeOH (4/1 volumes) was treated with LiOH (2.2-6 eq.) and stirred between RT and 50° C. for between 3 hrs and 18 hrs. The organic solvents were removed in vacuo then acidified with 1 M HCl and extracted with EtOAc. The organic phases were combined, dried (Na₂SO₄), filtered and concentrated. The products were used directly in the next step with no further purification undertaken.

Method F: Potassium Salt Formation

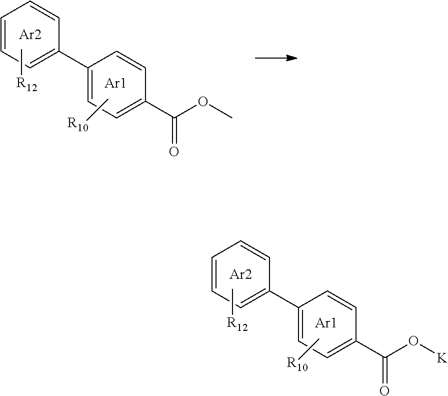

A solution of the ester (1 eq.) in THF (4 volumes) was treated with TMSOK (1 eq.) and stirred at RT for 2 hrs before the reaction mixtures were filtered and washed with iso-hexanes. The products were used directly in the next step with no further purification undertaken.

Compounds of Formula (I-c)—Examples

The synthesis of a number of known CTPS1 inhibitors is disclosed in VNO2019/106146 (see compounds R1 to R93). Such compounds are made using general methods disclosed herein and represent further examples of compounds which are CTPS1 inhibitors. The full synthetic methods and characterising data for compounds R1 to R93 are provided in WO2019/106146, which is herein incorporated in its entirety by reference.

Method 1: Amide Coupling

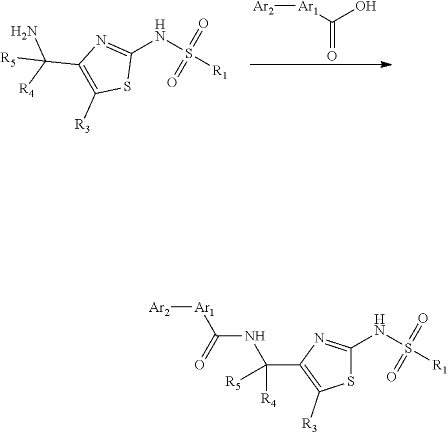

Method 1a: HATU (1.2 eq.) was added to a solution of appropriate acid (1 eq.), amine (1 eq.) and DIPEA (3 eq.) in DMF (10 volumes) at RT. The reaction was stirred at RT for 18 hrs. The solvent was removed and the crude product was purified by normal phase chromatography, reverse phase chromatography or trituration from an appropriate solvent.

Method 1b: 1-chloro-N,N,2-trimethylprop-1-en-1-amine (2 eq.) was added to a solution of appropriate acid (1 eq.) in DCM (20 volumes). The reaction mixture was stirred at RT for 2 hrs. The reaction mixture was concentrated in vacuo and the residue dissolved in DCM (20 volumes) before addition of DIPEA (3 eq.) and the appropriate amine (1 eq). The reaction mixture was stirred at RT for 2 hrs. An aqueous work up was performed and the crude product was purified by normal phase chromatography, reverse phase chromatography or trituration from an appropriate solvent.

Method 1c: T3P (50 wt % in EtOAc, 2.5 eq.) was added to a solution of appropriate acid (1 eq.), amine (1 eq.) and pyridine (3 eq.) in a mixture of EtOAc (20 volumes) and DMF (10 volumes). The reaction was stirred for 1 hr at RT. An aqueous work up was performed and the crude product was purified by normal phase chromatography, reverse phase chromatography or trituration from an appropriate solvent.

Method 2a: Suzuki [ArB(OR)₂ Core]

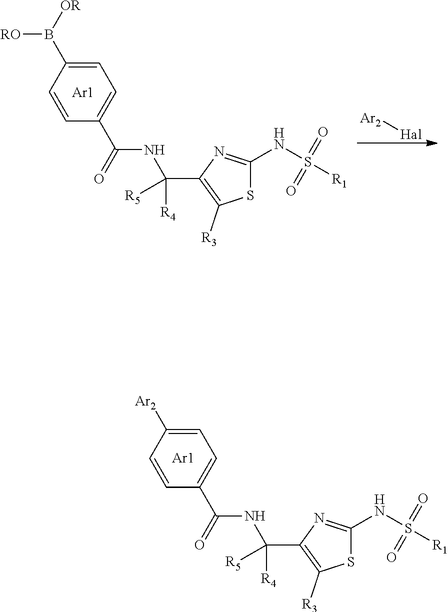

PdCl₂(dppf)-CH₂Cl₂ (10 mol %) or other appropriate catalyst was added to a degassed (N₂, 5 mins) solution of Ar1-B(OR)₂ (1 eq.), Ar2-halide (1 eq.) and K₂CO₃ (3 eq.) in dioxane (10 volumes) and water (1 volumes). The solution was then degassed further (N₂, 5 mins) and heated to 90° C. for 1-2 hrs. The reaction mixture was allowed to cool to RT. An aqueous workup was performed and the crude product was purified by normal phase chromatography, reverse phase chromatography or trituration from an appropriate solvent.

Method 2b: Telescoped Miyaura Borylation/Suzuki Protocol

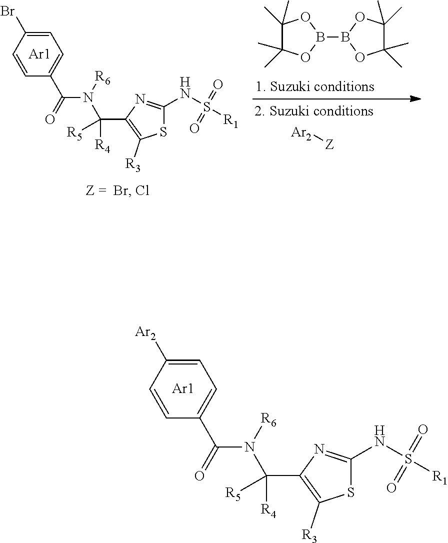

A suspension of Ar1-Br (1 eq.), Bispin (1.1 eq.) and KOAc (2 eq.) in dioxane (50 volumes) was degassed (N₂) then charged with PdCl₂(dppf).CH₂Cl₂ (5 mol %) and again degassed (N₂). The reaction mixture was heated to 90° C. for 1-24 hrs, recharging the Pd-catalyst if required. On formation of the boronate ester the reaction was allowed to cool to RT. Ar2-Z (1 eq.) and 2 M K₂CO₃ (eq, 2 eq.) were added, degassed (N₂) and the reaction was then heated to 90° C. for 18 hrs. The reaction was allowed to cool to RT, an aqueous work up was performed and the crude compound was purified by normal phase chromatography.

Representative for Method 1a
N-((2-(cyclopropanesulfonamido)thiazol-4-yl)methyl)-4-(pyridin-3-yl)benzamide R1

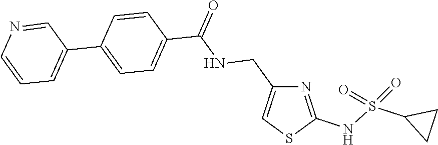 [SMILES: O=C(NCc1csc(NS(=O)(=O)C2CC2)n1)c1ccc(-c2cccnc2)cc1]

Representative for Method 1b
N-(1-(2-(cydopropanesulfonamido)thiazol-4-yl)propyl)-4-(5-(trifluoromethyl)pyridin-3-yl)benzamide R2

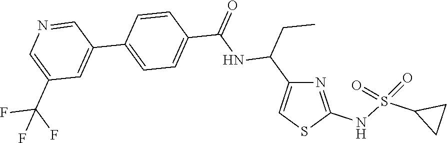 [SMILES: CCC(NC(=O)c1ccc(-c2cncc(C(F)(F)F)c2)cc1)c1csc(NS(=O)(=O)C2CC2)n1]

The racemic mixture R2 was separated by chiral preparative HPLC using chiral method A. A salt exchange (TFA to HCl) was undertaken by adding 1.25 M HCl (EtOH, 2 mL×5) and removing solvent to afford:

Peak 1: Stereochemistry of Product was not Defined R3
N-(1-(2-(cyclopropanesulfonamido)thiazol-4-yl)propyl)-4-(5-(trifluoromethyl)pyridin-3-yl)benzamide. HCl R3.

Peak 2: Stereochemistry of Product was not Defined R4
N-(1-(2-(cyclopropanesulfonamido)thiazol-4-yl)propyl)-4-(5-(trifluoromethyl)pyridin-3-yl)benzamide. HCl R4.

Representative for Method 1c
N-(1-(2-(cyclopropanesulfonamido)thiazol-4-yl)propyl)-4-(6-(trifluoromethyl)pyrazin-2-yl)benzamide R5

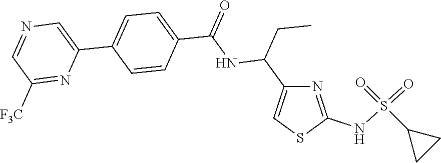 [SMILES: CCC(NC(=O)c1ccc(-c2cncc(C(F)(F)F)n2)cc1)c1csc(NS(=O)(=O)C2CC2)n1]

Representative for Method 2a
N-(2-(2-(cyclopropanesulfonamido)thiazol-4-yl)propan-2-yl)-4-(6-ethoxypyrazin-2-yl)-2-fluorobenzamide R6

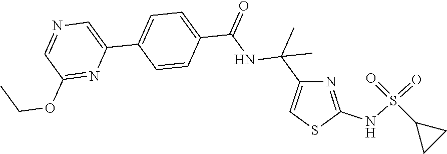 [SMILES: CCOc1cncc(-c2ccc(C(=O)NC(C)(C)c3csc(NS(=O)(=O)C4CC4)n3)cc2)n1]

Representative for Method 2b
N-(2-(2-(cyclopropanesulfonamido)thiazol-4-yl)propan-2-yl)-4-(6-ethoxypyrazin-2-yl)-2-methoxybenzamide R7

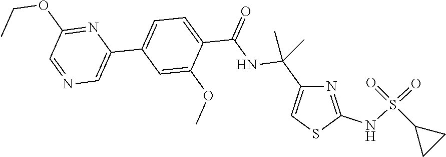 [SMILES: CCOc1cncc(-c2ccc(C(=O)NC(C)(C)c3csc(NS(=O)(=O)C4CC4)n3)c(OC)c2)n1]

TABLE 8
Compounds R8 to R93

| | Name/Structure |
|---|---|
| | (All examples containing chiral |
| R | centres are racemic unless stated) |
| R8 | N-((2-(cyclopropanesulfon- |
| | amido)thiazol-4-yl)methyl)-5- |
| | phenylpicolinamide |
| | 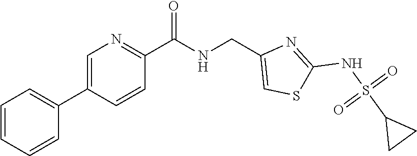 [SMILES: O=C(NCc1csc(NS(=O)(=O)C2CC2)n1)c1ccc(-c2ccccc2)cn1] |
| R9 | N-((2-(cyclopropanesulfon- |
| | amido)thiazol-4-ypmethyl)-[1,1′- |
| | biphenyl]-4-carboxamide |
| | 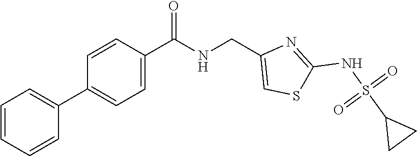 [SMILES: O=C(NCc1csc(NS(=O)(=O)C2CC2)n1)c1ccc(-c2ccccc2)cc1] |
| R10 | N-((2-(cyclopropanesulfon- |
| | amido)thiazol-4-yl)methyl)-2-fluoro-4- |
| | (6-(trifluoromethyl)-pyrazin-2- |
| | yl)benzamide |
| | 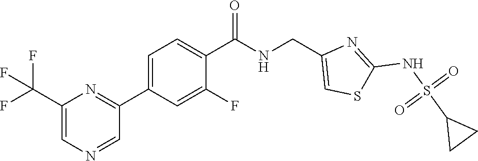 [SMILES: O=C(NCc1csc(NS(=O)(=O)C2CC2)n1)c1ccc(-c2cncc(C(F)(F)F)n2)cc1F] |
| R11 | N-((2-(cyclopropanesulfon- |
| | amido)thiazol-4-yl)methyl)-4-(6- |
| | ethoxypyrazin-2-yl)-2- |
| | fluorobenzamide |
| | 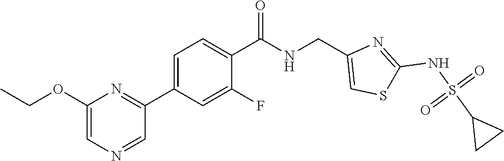 [SMILES: CCOc1cncc(-c2ccc(C(=O)NCc3csc(NS(=O)(=O)C4CC4)n3)c(F)c2)n1] |
| R12 | N-((2-(cyclopropanesulfon- |
| | amido)thiazol-4-yl)methyl)-4-(6- |
| | (trifluoromethyl)pyrazin-2- |
| | yl)benzamide |
| | 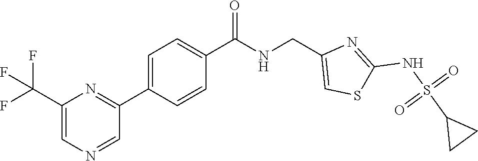 [SMILES: O=C(NCc1csc(NS(=O)(=O)C2CC2)n1)c1ccc(-c2cncc(C(F)(F)F)n2)cc1] |
| R13 | N-((2-(cyclopropanesulfon- |
| | amido)thiazol-4-yl)methyl)-4-(6- |
| | isopropoxypyrazin-2-yl)benzamide |
| | 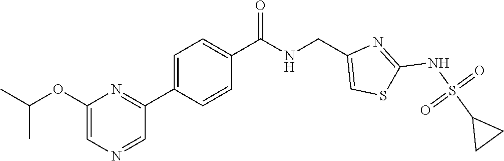 [SMILES: CC(C)Oc1cncc(-c2ccc(C(=O)NCc3csc(NS(=O)(=O)C4CC4)n3)cc2)n1] |
| R14 | N-((2-(cyclopropanesulfon- |
| | amido)thiazol-4-yl)methyl)-4-(6- |
| | ethoxypyrazin-2-yl)benzamide |
| | 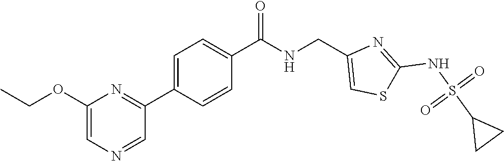 [SMILES: CCOc1cncc(-c2ccc(C(=O)NCc3csc(NS(=O)(=O)C4CC4)n3)cc2)n1] |
| R15 | N-(3-(2-(cyclopropanesulfon- |
| | amido)thiazol-4-yppentan-3-yl)-4-(5- |
| | (trifluoromethyl) pyridin-3- |
| | yl)benzamide |
| | 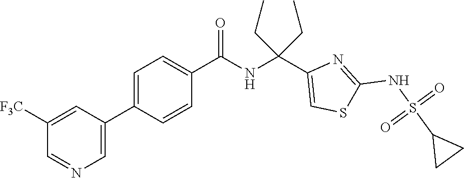 [SMILES: CCC(CC)(NC(=O)c1ccc(-c2cncc(C(F)(F)F)c2)cc1)c1csc(NS(=O)(=O)C2CC2)n1] |
| R16 | N-(3-(2-(cyclopropanesulfon- |
| | amido)thiazol-4-yppentan-3-yl)-4-(5- |
| | fluoropyridin-3-yl)benzamide |
| | 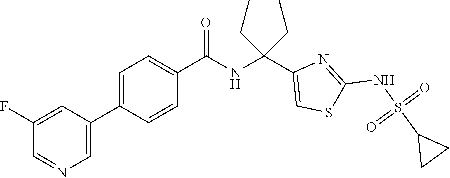 [SMILES: CCC(CC)(NC(=O)c1ccc(-c2cncc(F)c2)cc1)c1csc(NS(=O)(=O)C2CC2)n1] |
| R17 | N-(3-(2-(cyclopropanesulfon- |
| | amido)thiazol-4-yppentan-3-yl)-4-(5- |
| | methylpyridin-3-yl)benzamide |
| | 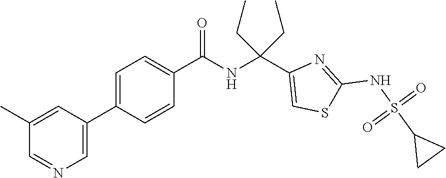 [SMILES: CCC(CC)(NC(=O)c1ccc(-c2cncc(C)c2)cc1)c1csc(NS(=O)(=O)C2CC2)n1] |
| R18 | N-(3-(2-(cyclopropanesulfon- |
| | amido)thiazol-4-yl)pentan-3-yl)-4- |
| | (pyridin-3-yl)benzamide |
| | 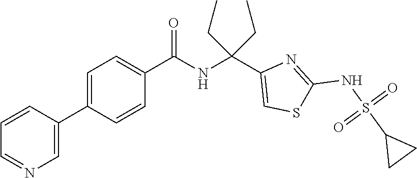 [SMILES: CCC(CC)(NC(=O)c1ccc(-c2cccnc2)cc1)c1csc(NS(=O)(=O)C2CC2)n1] |
| R19 | N-(3-(2-(cyclopropanesulfon- |
| | amido)thiazol-4-yl)pentan-3-yl)-4-(6- |
| | (trifluoromethyl) pyrazin-2- |
| | yl)benzamide |
| | 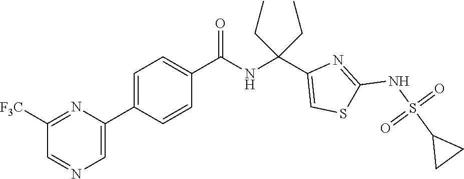 [SMILES: CCC(CC)(NC(=O)c1ccc(-c2cncc(C(F)(F)F)n2)cc1)c1csc(NS(=O)(=O)C2CC2)n1] |
| R20 | 4-(6-chloropyrazin-2-yl)-N-(3-(2- |
| | (cyclopropanesulfonamido) |
| | thiazol-4-yl)pentan-3-yl)benzamide |
| | 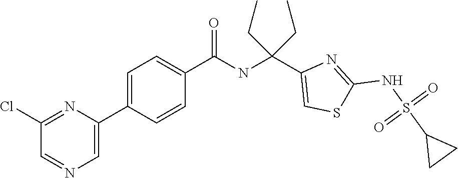 [SMILES: CCC(CC)(NC(=O)c1ccc(-c2cncc(Cl)n2)cc1)c1csc(NS(=O)(=O)C2CC2)n1] |
| R21 | N-(3-(2-(cyclopropanesulfon- |
| | amido)thiazol-4-yppentan-3-yl)-4-(6- |
| | methylpyrazin-2-yl)benzamide |
| | 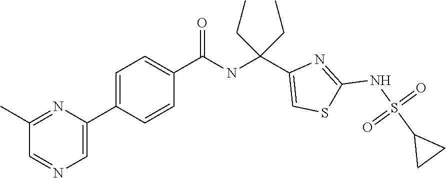 [SMILES: CCC(CC)(NC(=O)c1ccc(-c2cncc(C)n2)cc1)c1csc(NS(=O)(=O)C2CC2)n1] |
| R22 | N-(3-(2-(cyclopropanesulfon- |
| | amido)thiazol-4-yl)pentan-3-yl)-4- |
| | (pyrazin-2-yl)benzamide |
| | 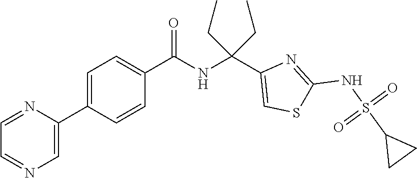 [SMILES: CCC(CC)(NC(=O)c1ccc(-c2cnccn2)cc1)c1csc(NS(=O)(=O)C2CC2)n1] |
| R23 | N-(2-(2-(cyclopropanesulfon- |
| | amido)thiazol-4-yl)propan-2-yl)-5-(6- |
| | ethoxypyrazin-2-yl)-3- |
| | fluoropicolinamide |
| | 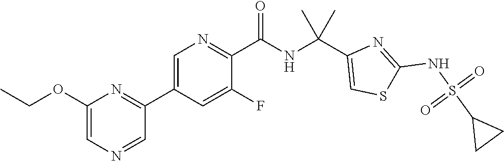 [SMILES: CCOc1cncc(-c2cnc(C(=O)NC(C)(C)c3csc(NS(=O)(=O)C4CC4)n3)c(F)c2)n1] |
| R24 | N-(2-(2-(cyclopropanesulfon- |
| | amido)thiazol-4-y0propan-2-yl)-5-(6- |
| | (trifluoromethyl)pyrazin-2- |
| | yl)picolinamide |
| | 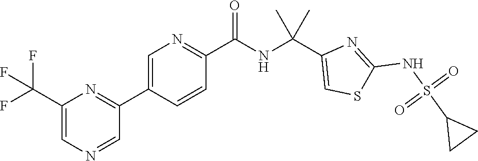 [SMILES: CC(C)(NC(=O)c1ccc(-c2cncc(C(F)(F)F)n2)cn1)c1csc(NS(=O)(=O)C2CC2)n1] |
| R25 | 5-(6-chloropyrazin-2-yl)-N-(2-(2- |
| | (cyclopropanesulfonamido) |
| | thiazol-4-yl)propan-2-yl)picolinamide |
| | 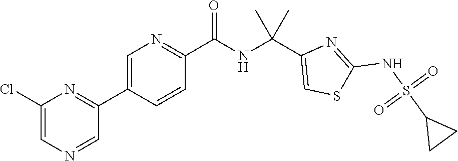 [SMILES: CC(C)(NC(=O)c1ccc(-c2cncc(Cl)n2)cn1)c1csc(NS(=O)(=O)C2CC2)n1] |
| R26 | N-(2-(2-(cyclopropanesulfon- |
| | amido)thiazol-4-yl)propan-2-yl)-5-(6- |
| | ethoxypyrazin-2-yl)picolinamide |
| | 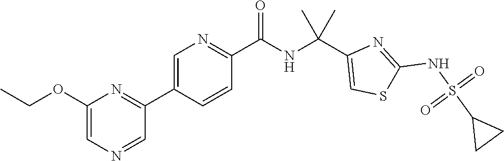 [SMILES: CCOc1cncc(-c2ccc(C(=O)NC(C)(C)c3csc(NS(=O)(=O)C4CC4)n3)nc2)n1] |
| R27 | N-(2-(2-(cyclopropanesulfon- |
| | amido)thiazol-4-yl)propan-2-yl)-[2,2′- |
| | bipyridine]-5-carboxamide |
| | 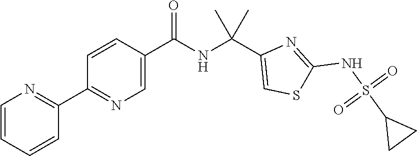 [SMILES: CC(C)(NC(=O)c1ccc(-c2ccccn2)nc1)c1csc(NS(=O)(=O)C2CC2)n1] |
| R28 | 4-(5-chloropyridin-3-yl)-N-(2-(2- |
| | (cyclopropanesulfon-amido)thiazol-4- |
| | yl)propan-2-yl)benzamide |
| | 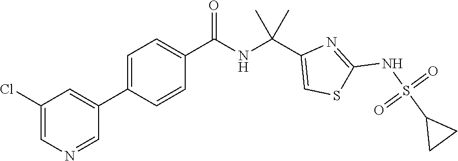 [SMILES: CC(C)(NC(=O)c1ccc(-c2cncc(Cl)c2)cc1)c1csc(NS(=O)(=O)C2CC2)n1] |
| R29 | N-(2-(2-(cyclopropanesulfon- |
| | amido)thiazol-4-yl)propan-2-yl)-2- |
| | fluoro-4-(5-(trifluoromethyl)pyridin-3- |
| | yl)benzamide |
| | 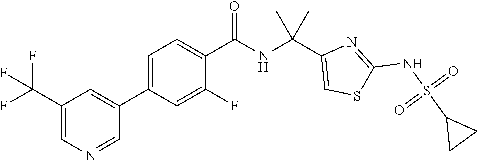 [SMILES: CC(C)(NC(=O)c1ccc(-c2cncc(C(F)(F)F)c2)cc1F)c1csc(NS(=O)(=O)C2CC2)n1] |
| R30 | 4-(5-chloropyridin-3-yl)-N-(2-(2- |
| | (cyclopropanesulfon-amido)thiazol-4- |
| | yl)propan-2-yl)-2-fluorobenzamide |
| | 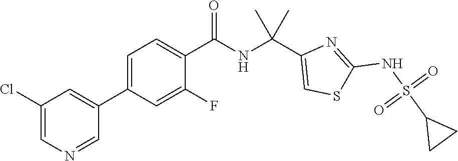 [SMILES: CC(C)(NC(=O)c1ccc(-c2cncc(Cl)c2)cc1F)c1csc(NS(=O)(=O)C2CC2)n1] |
| R31 | N-(2-(2-(cyclopropanesulfon- |
| | amido)thiazol-4-yl)propan-2-yl)-2- |
| | fluoro-4-(5-fluoropyridin-3- |
| | yl)benzamide |
| | 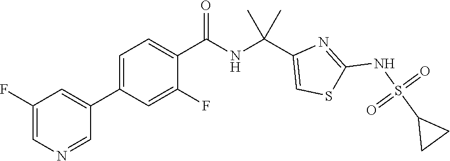 [SMILES: CC(C)(NC(=O)c1ccc(-c2cncc(F)c2)cc1F)c1csc(NS(=O)(=O)C2CC2)n1] |
| R32 | N-(2-(2-(cyclopropanesulfon- |
| | amido)thiazol-4-yl)propan-2-yl)-2- |
| | methoxy-4-(5-(trifluoromethyl)pyridin- |
| | 3-yl)benzamide |
| | 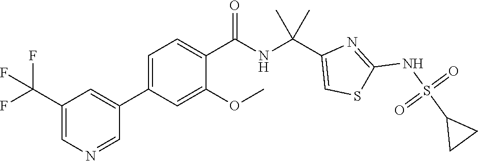 [SMILES: COc1cc(-c2cncc(C(F)(F)F)c2)ccc1C(=O)NC(C)(C)c1csc(NS(=O)(=O)C2CC2)n1] |
| R33 | N-(2-(2- |
| | (cyclopropanesulfonamido)thiazol-4- |
| | yl)propan-2-yl)-4-(2-methylpyridin-3- |
| | yl)benzamide |
| | 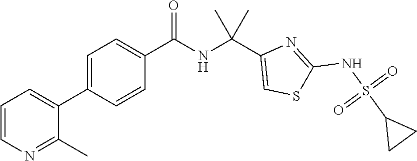 [SMILES: Cc1ncccc1-c1ccc(C(=O)NC(C)(C)c2csc(NS(=O)(=O)C3CC3)n2)cc1] |
| R34 | 4-(5-acetylpyridin-3-yl)-N-(2-(2- |
| | (cyclopropanesulfon-amido)thiazol-4- |
| | yl)propan-2-yl)benzamide |
| | 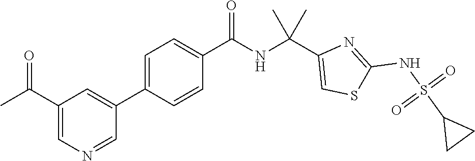 [SMILES: CC(=O)c1cncc(-c2ccc(C(=O)NC(C)(C)c3csc(NS(=O)(=O)C4CC4)n3)cc2)c1] |
| R35 | N-(2-(2-(cyclopropanesulfon- |
| | amido)thiazol-4-yl)propan-2-yl)-4-(5- |
| | (trifluoromethyl)pyridin-3- |
| | yl)benzamide |
| | 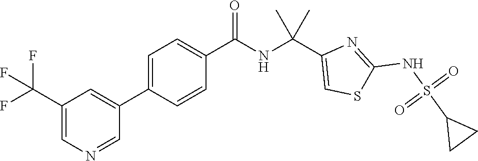 [SMILES: CC(C)(NC(=O)c1ccc(-c2cncc(C(F)(F)F)c2)cc1)c1csc(NS(=O)(=O)C2CC2)n1] |
| R36 | N-(2-(2-(cyclopropanesulfon- |
| | amido)thiazol-4-yppropan-2-yl)-4-(5- |
| | fluoropyridin-3-yl)benzamide |
| | 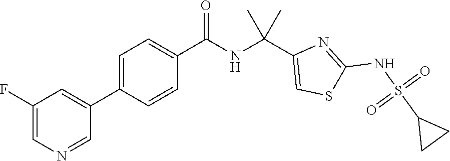 [SMILES: CC(C)(NC(=O)c1ccc(-c2cncc(F)c2)cc1)c1csc(NS(=O)(=O)C2CC2)n1] |
| R37 | N-(2-(2-(cyclopropanesulfon- |
| | amido)thiazol-4-yl)propan-2-yl)-4-(5- |
| | methylpyridin-3-yl)benzamide |
| | 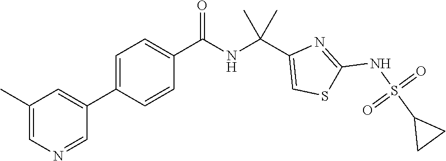 [SMILES: Cc1cncc(-c2ccc(C(=O)NC(C)(C)c3csc(NS(=O)(=O)C4CC4)n3)cc2)c1] |
| R38 | N-(2-(2-(cyclopropanesulfon- |
| | amido)thiazol-4-yppropan-2-yl)-4-(5- |
| | methoxypyridin-3-yl)benzamide |
| | 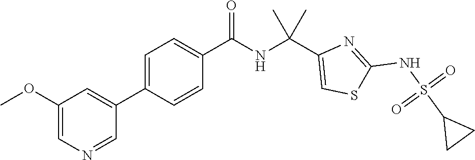 [SMILES: COc1cncc(-c2ccc(C(=O)NC(C)(C)c3csc(NS(=O)(=O)C4CC4)n3)cc2)c1] |
| R39 | N-(2-(2-(cyclopropanesulfon- |
| | amido)thiazol-4-yl)propan-2-yl)-4- |
| | (pyridin-3-yl)benzamide |
| | 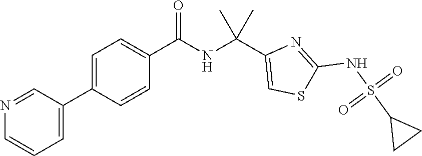 [SMILES: CC(C)(NC(=O)c1ccc(-c2cccnc2)cc1)c1csc(NS(=O)(=O)C2CC2)n1] |
| R40 | N-(2-(2-(cyclopropanesulfon- |
| | amido)thiazol-4-yl)propan-2-yl)-3′- |
| | (trifluoromethyl)-[1,1′-biphenyl]-4- |
| | carboxamide |
| | 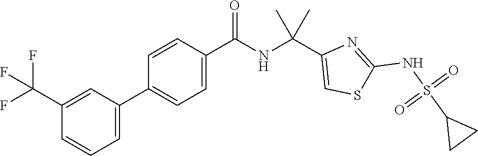 [SMILES: CC(C)(NC(=O)c1ccc(-c2cccc(C(F)(F)F)c2)cc1)c1csc(NS(=O)(=O)C2CC2)n1] |
| R41 | N-(2-(2-(cyclopropanesulfon- |
| | amido)thiazol-4-yl)propan-2-yl)-4-(6- |
| | ethylpyrazin-2-yl)-2-fluorobenzamide |
| | 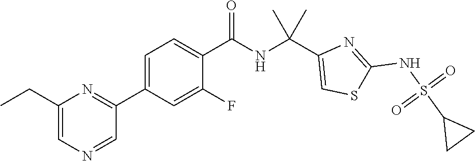 [SMILES: CCc1cncc(-c2ccc(C(=O)NC(C)(C)c3csc(NS(=O)(=O)C4CC4)n3)c(F)c2)n1] |
| R42 | N-(2-(2-(cyclopropane |
| | sulfonamido)thiazol-4-yl)propan-2-yl)- |
| | 2-fluoro-4-(6-(trifluoromethyl)pyrazin- |
| | 2-yl)benzamide |
| | 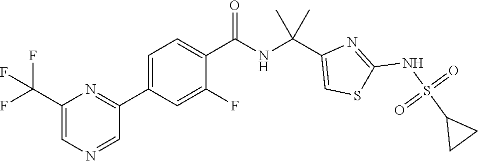 [SMILES: CC(C)(NC(=O)c1ccc(-c2cncc(C(F)(F)F)n2)cc1F)c1csc(NS(=O)(=O)C2CC2)n1] |
| R43 | N-(2-(2-(cyclopropane |
| | sulfonamido)thiazol-4-yl)propan-2-yl)- |
| | 2-fluoro-4-(6-isopropoxypyrazin-2- |
| | yl)benzamide |
| | 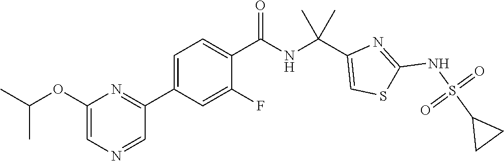 [SMILES: CC(C)Oc1cncc(-c2ccc(C(=O)NC(C)(C)c3csc(NS(=O)(=O)C4CC4)n3)c(F)c2)n1] |
| R44 | N-(2-(2-(cyclopropanesulfon |
| | amido)thiazol-4-yl)propan-2-yl)-2- |
| | fluoro-4-(6-(2,2,2- |
| | trifluoroethoxy)pyrazin-2- |
| | yl)benzamide |
| | 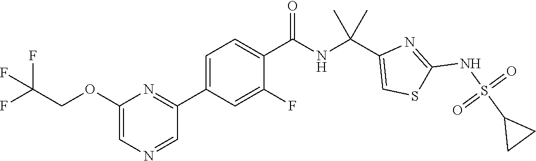 [SMILES: CC(C)(NC(=O)c1ccc(-c2cncc(OCC(F)(F)F)n2)cc1F)c1csc(NS(=O)(=O)C2CC2)n1] |
| R45 | N-(2-(2-(cyclopropane |
| | sulfonamido)thiazol-4-yl)propan-2-yl)- |
| | 2-methyl-4-(6- |
| | (trifluoromethyl)pyrazin-2- |
| | yl)benzamide |
| | 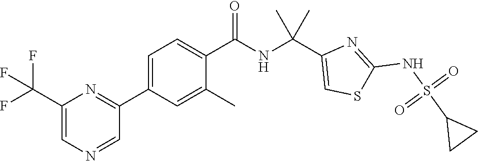 [SMILES: Cc1cc(-c2cncc(C(F)(F)F)n2)ccc1C(=O)NC(C)(C)c1csc(NS(=O)(=O)C2CC2)n1] |
| R46 | N-(2-(2- |
| | (cyclopropanesulfonamido)thiazol-4- |
| | yl)propan-2-yl)-4-(6-ethoxypyrazin-2- |
| | yl)-2-methyl benzamide |
| | 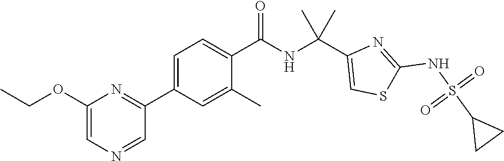 [SMILES: CCOc1cncc(-c2ccc(C(=O)NC(C)(C)c3csc(NS(=O)(=O)C4CC4)n3)c(C)c2)n1] |
| R47 | N-(2-(2- |
| | (cyclopropanesulfonamido)thiazol-4- |
| | yl)propan-2-yl)-4-(6-ethoxypyrazin-2- |
| | yl)-2-(trifluoromethyl)benzamide |
| | 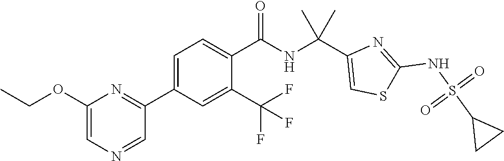 [SMILES: CCOc1cncc(-c2ccc(C(=O)NC(C)(C)c3csc(NS(=O)(=O)C4CC4)n3)c(C(F)(F)F)c2)n1] |
| R48 | N-(2-(2- |
| | (cyclopropanesulfonamido)thiazol-4- |
| | yl)propan-2-yl)-2-methoxy-4-(6- |
| | (trifluoromethyl)pyrazin-2- |
| | yl)benzamide |
| | 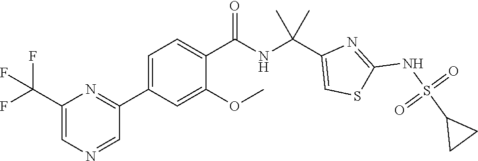 [SMILES: COc1cc(-c2cncc(C(F)(F)F)n2)ccc1C(=O)NC(C)(C)c1csc(NS(=O)(=O)C2CC2)n1] |
| R49 | 4-(6-chloropyrazin-2-yl)-N-(2-(2- |
| | (cyclopropanesulfonamido)thiazol-4- |
| | yl)propan-2-yl)-2-methoxybenzamide |
| | 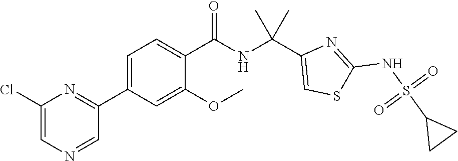 [SMILES: COc1cc(-c2cncc(Cl)n2)ccc1C(=O)NC(C)(C)c1csc(NS(=O)(=O)C2CC2)n1] |
| R50 | 4-(6-cyanopyrazin-2-yl)-N-(2-(2- |
| | (cyclopropanesulfonamido)thiazol-4- |
| | yl)propan-2-yl)-2-methoxybenzamide |
| | 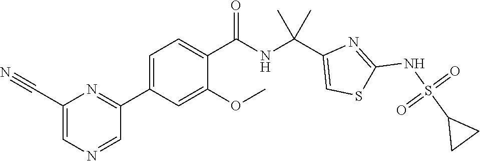 [SMILES: COc1cc(-c2cncc(C#N)n2)ccc1C(=O)NC(C)(C)c1csc(NS(=O)(=O)C2CC2)n1] |
| R51 | N-(2-(2-(cyclopropanesulfon- |
| | amido)thiazol-4-yl)propan-2-yl)-4-(6- |
| | (trifluoromethyl) pyrazin-2-yl)benzamide |
| | 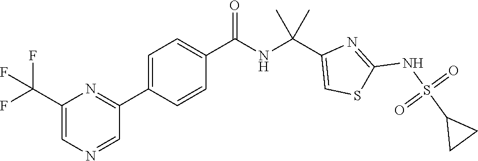 [SMILES: CC(C)(NC(=O)c1ccc(-c2cncc(C(F)(F)F)n2)cc1)c1csc(NS(=O)(=O)C2CC2)n1] |
| R52 | 4-(6-chloropyrazin-2-yl)-N-(2-(2- |
| | (cyclopropanesulfon-amido)thiazol-4- |
| | yl)propan-2-yl)benzamide |
| | 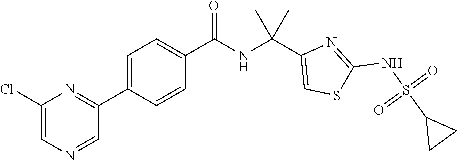 [SMILES: CC(C)(NC(=O)c1ccc(-c2cncc(Cl)n2)cc1)c1csc(NS(=O)(=O)C2CC2)n1] |
| R53 | N-(2-(2-(cyclopropanesulfon- |
| | amido)thiazol-4-yl)propan-2-yl)-4-(6- |
| | methylpyrazin-2-yl)benzamide |
| | 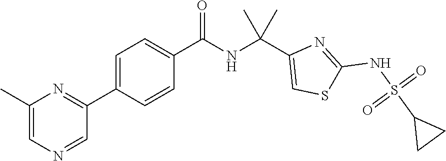 [SMILES: Cc1cncc(-c2ccc(C(=O)NC(C)(C)c3csc(NS(=O)(=O)C4CC4)n3)cc2)n1] |
| R54 | N-(2-(2-(cyclopropanesulfon- |
| | amido)thiazol-4-yl)propan-2-yl)-4-(6- |
| | methoxypyrazin-2-yl)benzamide |
| | 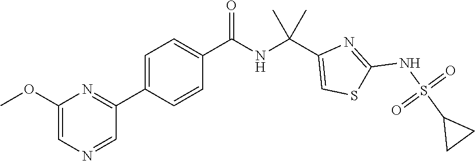 [SMILES: COc1cncc(-c2ccc(C(=O)NC(C)(C)c3csc(NS(=O)(=O)C4CC4)n3)cc2)n1] |
| R55 | N-(2-(2-(cyclopropanesulfon- |
| | amido)thiazol-4-yl)propan-2-yl)-4-(6- |
| | ethoxypyrazin-2-yl)benzamide |
| | 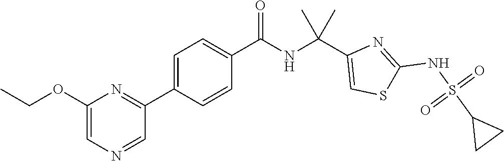 [SMILES: CCOc1cncc(-c2ccc(C(=O)NC(C)(C)c3csc(NS(=O)(=O)C4CC4)n3)cc2)n1] |
| R56 | N-(2-(2-(cyclopropanesulfon- |
| | amido)thiazol-4-yl)propan-2-yl)-4-(6- |
| | isopropoxypyrazin-2-yl)benzamide |
| | 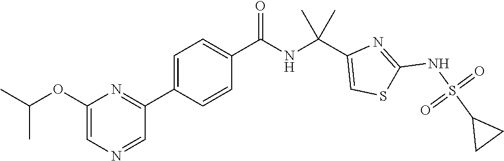 [SMILES: CC(C)Oc1cncc(-c2ccc(C(=O)NC(C)(C)c3csc(NS(=O)(=O)C4CC4)n3)cc2)n1] |
| R57 | N-(2-(2-(cyclopropanesulfon- |
| | amido)thiazol-4-yl)propan-2-yl)-4-(6- |
| | (2,2,2-trifluoroethoxy)pyrazin-2- |
| | yl)benzamide |
| | 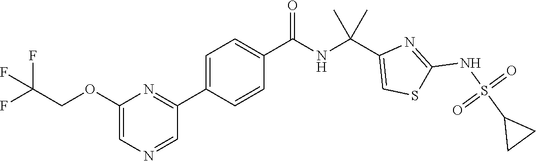 [SMILES: CC(C)(NC(=O)c1ccc(-c2cncc(OCC(F)(F)F)n2)cc1)c1csc(NS(=O)(=O)C2CC2)n1] |
| R58 | N-(2-(2-(cyclopropanesulfon- |
| | amido)thiazol-4-yl)propan-2-yl)-4- |
| | (pyrazin-2-yl)benzamide |
| | 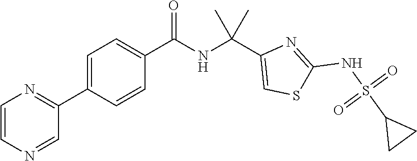 [SMILES: CC(C)(NC(=O)c1ccc(-c2cnccn2)cc1)c1csc(NS(=O)(=O)C2CC2)n1] |
| R59 | RACEMIC, N-(1-(2- |
| | (cyclopropanesulfonamido)thiazol-4- |
| | yl)propyl)-4-(5-fluoropyridin-3- |
| | yl)benzamide |
| | 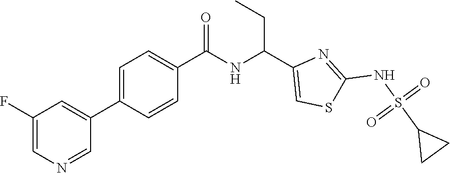 [SMILES: CCC(NC(=O)c1ccc(-c2cncc(F)c2)cc1)c1csc(NS(=O)(=O)C2CC2)n1] |
| R60 | RACEMIC, N-(1-(2- |
| | (cyclopropanesulfonamido)thiazol-4- |
| | yl)propyl)-4-(5-methylpyridin-3- |
| | yl)benzamide |
| | 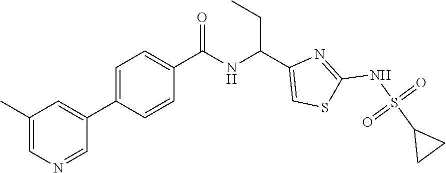 [SMILES: CCC(NC(=O)c1ccc(-c2cncc(C)c2)cc1)c1csc(NS(=O)(=O)C2CC2)n1] |
| R61 | RACEMIC, N-(1-(2- |
| | (cyclopropanesulfonamido)thiazol-4- |
| | Apropyl)-4-(pyridin-3-yl)benzamide |
| | 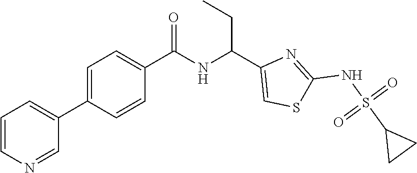 [SMILES: CCC(NC(=O)c1ccc(-c2cccnc2)cc1)c1csc(NS(=O)(=O)C2CC2)n1] |
| R62 | RACEMIC, N-(1-(2- |
| | (cyclopropanesulfonamido)thiazol-4- |
| | yl)propyl)-4-(6-ethoxypyrazin-2-yl)-2- |
| | fluorobenzamide |
| | 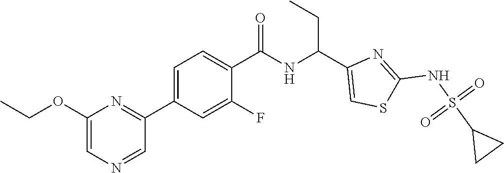 [SMILES: CCOc1cncc(-c2ccc(C(=O)NC(CC)c3csc(NS(=O)(=O)C4CC4)n3)c(F)c2)n1] |
| R63 | RACEMIC, N-(1-(2- |
| | (cyclopropanesulfonamido)thiazol-4- |
| | yl)propyl)-4-(6-ethoxypyrazin-2-yl)-2- |
| | fluoro-N-methylbenzamide |
| | 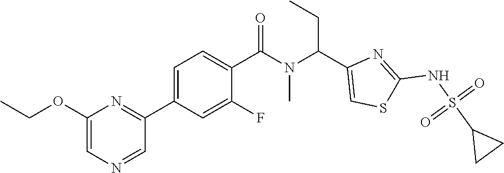 [SMILES: CCOc1cncc(-c2ccc(C(=O)N(C)C(CC)c3csc(NS(=O)(=O)C4CC4)n3)c(F)c2)n1] |
| R64 | RACEMIC, N-(1-(2- |
| | (cyclopropanesulfonamido)thiazol-4- |
| | yl)propyl)-2-fluoro-4-(6- |
| | isopropoxypyrazin-2-yl)benzamide |
| | 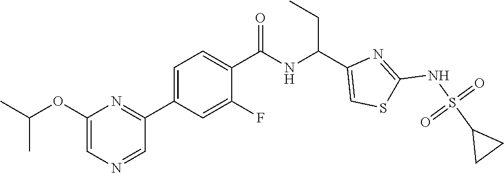 [SMILES: CCC(NC(=O)c1ccc(-c2cncc(OC(C)C)n2)cc1F)c1csc(NS(=O)(=O)C2CC2)n1] |
| R65 | RACEMIC, 4-(6-chloropyrazin-2-yl)-N- |
| | (1-(2-(cyclopropanesulfonamido)thiazol- |
| | 4-yl)propyl)benzamide |
| | 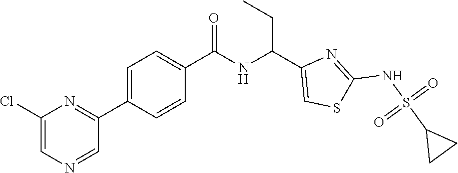 [SMILES: CCC(NC(=O)c1ccc(-c2cncc(Cl)n2)cc1)c1csc(NS(=O)(=O)C2CC2)n1] |
| R66 | RACEMIC, N-(1-(2- |
| | (cyclopropanesulfonamido)thiazol-4- |
| | yl)propyl)-4-(6-methylpyrazin-2- |
| | yl)benzamide |
| | 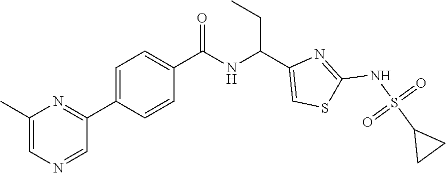 [SMILES: CCC(NC(=O)c1ccc(-c2cncc(C)n2)cc1)c1csc(NS(=O)(=O)C2CC2)n1] |
| R67 | RACEMIC, N-(1-(2- |
| | (cyclopropanesulfonamido)thiazol-4- |
| | yl)propyl)-4-(pyrazin-2-yl)benzamide |
| | 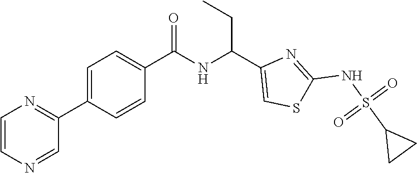 [SMILES: CCC(NC(=O)c1ccc(-c2cnccn2)cc1)c1csc(NS(=O)(=O)C2CC2)n1] |
| R68 | SINGLE ENANTIOMER - |
| | stereochemistry unassigned, N-(1-(2- |
| | (cyclopropanesulfon-amido)thiazol-4- |
| | yl)propyl)-4-(5-fluoropyridin-3- |
| | yl)benzamide |
| | 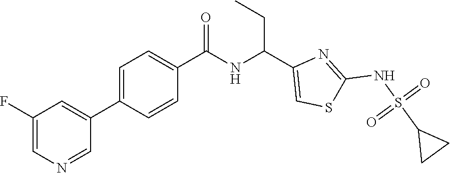 [SMILES: CCC(NC(=O)c1ccc(-c2cncc(F)c2)cc1)c1csc(NS(=O)(=O)C2CC2)n1] |
| R69 | SINGLE ENANTIOMER - |
| | stereochemistry unassigned, N-(1-(2- |
| | (cyclopropanesulfon-amido)thiazol-4- |
| | yl)propyl)-4-(5-fluoropyridin-3- |
| | yl)benzamide |
| | 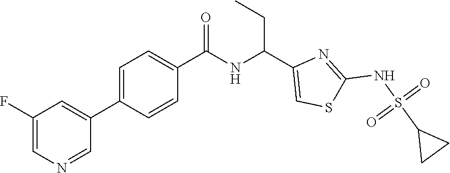 [SMILES: CCC(NC(=O)c1ccc(-c2cncc(F)c2)cc1)c1csc(NS(=O)(=O)C2CC2)n1] |
| R70 | SINGLE ENANTIOMER - |
| | stereochemistry unassigned, N-(1-(2- |
| | (cyclopropanesulfon-amido)thiazol-4- |
| | yl)propyl)-4-(6-ethoxypyrazin-2-yl)-2- |
| | fluorobenzamide |
| | 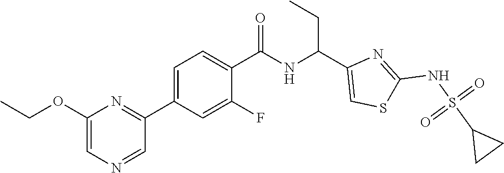 [SMILES: CCOc1cncc(-c2ccc(C(=O)NC(CC)c3csc(NS(=O)(=O)C4CC4)n3)c(F)c2)n1] |
| R71 | SINGLE ENANTIOMER - |
| | stereochemistry unassigned, N-(1-(2- |
| | (cyclopropanesulfon-amido)thiazol-4- |
| | yl)propyl)-4-(6-ethoxypyrazin-2-yl)-2- |
| | fluorobenzamide |
| | 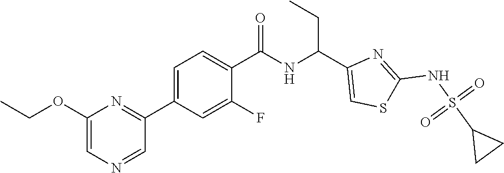 [SMILES: CCOc1cncc(-c2ccc(C(=O)NC(CC)c3csc(NS(=O)(=O)C4CC4)n3)c(F)c2)n1] |
| R72 | N-(2-(2-(cyclopropanesulfon-amido)-5- |
| | methylthiazol-4-yl)propan-2-yl)-5-(6- |
| | ethoxypyrazin-2-yl)picolinamide |
| | 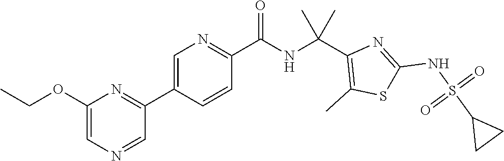 [SMILES: CCOc1cncc(-c2ccc(C(=O)NC(C)(C)c3nc(NS(=O)(=O)C4CC4)sc3C)nc2)n1] |
| R73 | N-(2-(5-chloro-2-(cycloprop- |
| | anesulfonamido)thiazol-4-Apropan-2- |
| | yl)-5-(6-ethoxypyrazin-2-yl)picolinamide |
| | 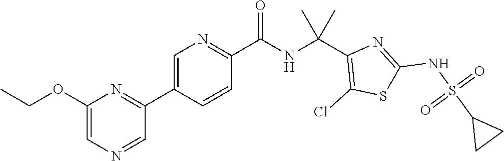 [SMILES: CCOc1cncc(-c2ccc(C(=O)NC(C)(C)c3nc(NS(=O)(=O)C4CC4)sc3Cl)nc2)n1] |
| R74 | N-(2-(2-(cyclopropanesulfon-amido)-5- |
| | methylthiazol-4-yl)propan-2-yl)-4-(6- |
| | ethoxypyrazin-2-yl)-2-fluorobenzamide |
| | 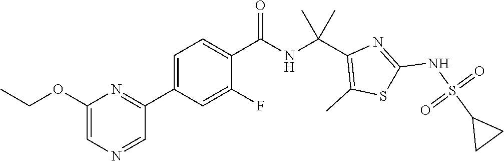 [SMILES: CCOc1cncc(-c2ccc(C(=O)NC(C)(C)c3nc(NS(=O)(=O)C4CC4)sc3C)c(F)c2)n1] |
| R75 | N-(2-(5-chloro-2-(cycloprop- |
| | anesulfonamido)thiazol-4-yl)propan-2- |
| | yl)-4-(6-ethoxypyrazin-2-yl)-2- |
| | fluorobenzamide |
| | 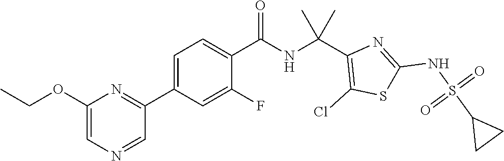 [SMILES: CCOc1cncc(-c2ccc(C(=O)NC(C)(C)c3nc(NS(=O)(=O)C4CC4)sc3Cl)c(F)c2)n1] |
| R76 | N-(2-(2-(cyclopropanesulfon-amido)-5- |
| | methylthiazol-4-yl)propan-2-yl)-2- |
| | methyl-4-(6-(trifluoromethyl)pyrazin-2- |
| | yl)benzamide |
| | 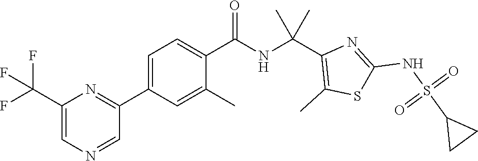 [SMILES: Cc1cc(-c2cncc(C(F)(F)F)n2)ccc1C(=O)NC(C)(C)c1nc(NS(=O)(=O)C2CC2)sc1C] |
| R77 | N-(2-(5-chloro-2-(cycloprop- |
| | anesulfonamido)thiazol-4-Apropan-2- |
| | yl)-2-methyl-4-(6- |
| | (trifluoromethyl)pyrazin-2-yl)benzamide |
| | 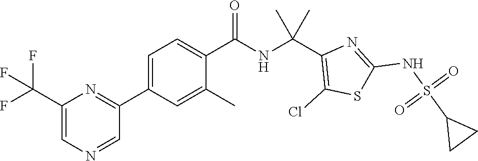 [SMILES: Cc1cc(-c2cncc(C(F)(F)F)n2)ccc1C(=O)NC(C)(C)c1nc(NS(=O)(=O)C2CC2)sc1Cl] |
| R78 | N-(2-(2-(cyclopropanesulfon-amido)-5- |
| | methylthiazol-4-yl)propan-2-yl)-4-(6- |
| | (trifluoromethyl)pyrazin-2-yl)benzamide |
| | 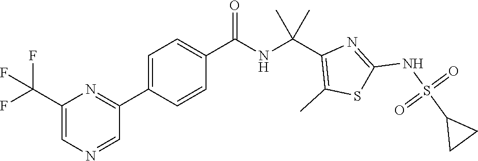 [SMILES: Cc1sc(NS(=O)(=O)C2CC2)nc1C(C)(C)NC(=O)c1ccc(-c2cncc(C(F)(F)F)n2)cc1] |
| R79 | N-(2-(5-chloro-2-(cycloprop- |
| | anesulfonamido)thiazol-4-yl)propan-2- |
| | yl)-4-(6-(trifluoromethyl)pyrazin-2- |
| | yl)benzamide |
| | 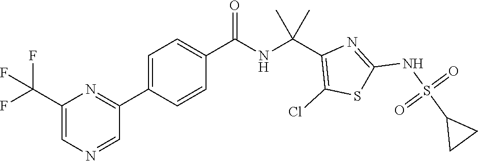 [SMILES: CC(C)(NC(=O)c1ccc(-c2cncc(C(F)(F)F)n2)cc1)c1nc(NS(=O)(=O)C2CC2)sc1Cl] |
| R80 | N-(1-(2-(cyclopropanesulfon- |
| | amido)thiazol-4-yl)cyclo-propyl)-5-(6- |
| | ethoxypyrazin-2-yl)picolinamide |
| | 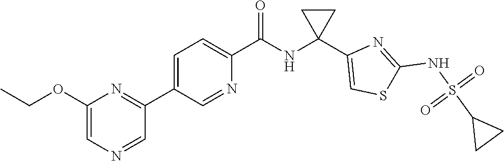 [SMILES: CCOc1cncc(-c2ccc(C(=O)NC3(c4csc(NS(=O)(=O)C5CC5)n4)CC3)nc2)n1] |
| R81 | N-(1-(2-(cyclopropanesulfon- |
| | amido)thiazol-4-yl)cyclo-propyl)-4- |
| | (pyridin-3-yl)benzamide |
| | 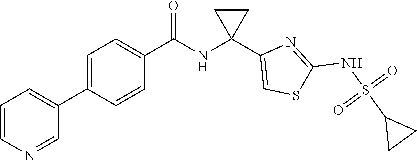 [SMILES: O=C(NC1(c2csc(NS(=O)(=O)C3CC3)n2)CC1)c1ccc(-c2cccnc2)cc1] |
| R82 | N-(1-(2-(cyclopropanesulfon- |
| | amido)thiazol-4-yl)cyclo-propyl)-4-(6- |
| | ethoxypyrazin-2-yl)-2-fluorobenzamide |
| | 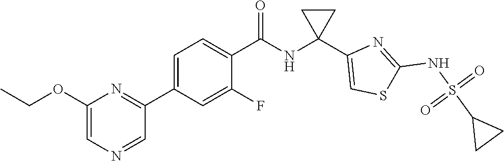 [SMILES: CCOc1cncc(-c2ccc(C(=O)NC3(c4csc(NS(=O)(=O)C5CC5)n4)CC3)c(F)c2)n1] |
| R83 | N-(1-(2-(cyclopropanesulfon- |
| | amido)thiazol-4-yl)cyclopropyl)-2- |
| | methyl-4-(6-(trifluoromethyl)pyrazin-2- |
| | yl)benzamide |
| | 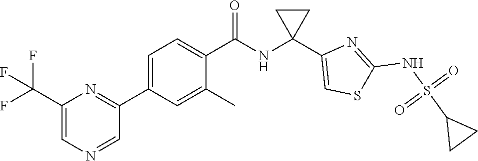 [SMILES: Cc1cc(-c2cncc(C(F)(F)F)n2)ccc1C(=O)NC1(c2csc(NS(=O)(=O)C3CC3)n2)CC1] |
| R84 | N-(1-(2-(cyclopropanesulfon- |
| | amido)thiazol-4-yl)cyclopropyl)-4-(6- |
| | (trifluoromethyl)pyrazin-2-yl)benzamide |
| | 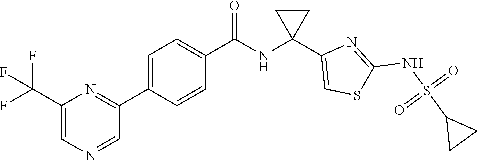 [SMILES: O=C(NC1(c2csc(NS(=O)(=O)C3CC3)n2)CC1)c1ccc(-c2cncc(C(F)(F)F)n2)cc1] |
| R85 | N-(1-(2-(cyclopropanesulfon- |
| | amido)thiazol-4-yl)-3-methoxypropyl)-4- |
| | (5-fluoropyridin-3-yl)benzamide |
| | 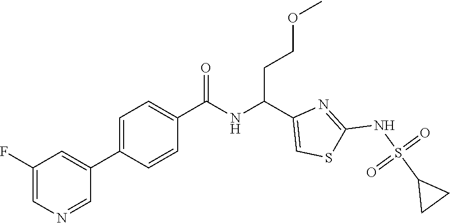 [SMILES: COCCC(NC(=O)c1ccc(-c2cncc(F)c2)cc1)c1csc(NS(=O)(=O)C2CC2)n1] |
| R86 | N-(1-(2-(cyclopropanesulfon- |
| | amido)thiazol-4-yl)-3-methoxypropyl)-4- |
| | (6-ethyl-pyrazin-2-yl)-2- |
| | fluorobenzamide |
| | 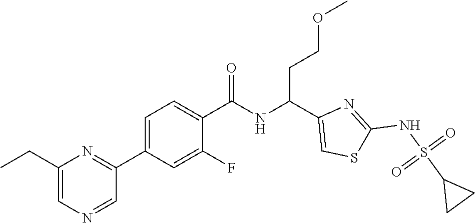 [SMILES: CCc1cncc(-c2ccc(C(=O)NC(CCOC)c3csc(NS(=O)(=O)C4CC4)n3)c(F)c2)n1] |
| R87 | N-(1-(2-(cyclopropanesulfon- |
| | amido)thiazol-4-yl)-3-methoxypropyl)-2- |
| | fluoro-4-(6-(trifluoromethyl)pyrazin-2- |
| | yl)benzamide |
| | 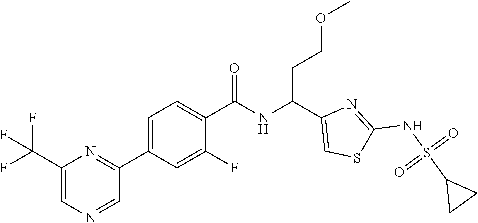 [SMILES: COCCC(NC(=O)c1ccc(-c2cncc(C(F)(F)F)n2)cc1F)c1csc(NS(=O)(=O)C2CC2)n1] |
| R88 | N-(1-(2-(cyclopropanesulfon- |
| | amido)thiazol-4-yl)-3-methoxypropyl)-4- |
| | (6-ethoxypyrazin-2-yl)-2- |
| | fluorobenzamide |
| | 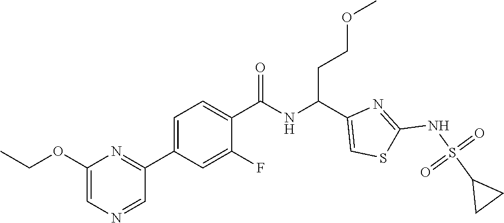 [SMILES: CCOc1cncc(-c2ccc(C(=O)NC(CCOC)c3csc(NS(=O)(=O)C4CC4)n3)c(F)c2)n1] |
| R89 | N-(1-(2-(cyclopropanesulfon- |
| | amido)thiazol-4-yl)-3-methoxypropyl)-2- |
| | fluoro-4-(6-isopropoxypyrazin-2- |
| | yl)benzamide |
| | 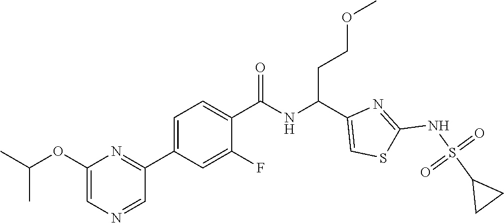 [SMILES: COCCC(NC(=O)c1ccc(-c2cncc(OC(C)C)n2)cc1F)c1csc(NS(=O)(=O)C2CC2)n1] |
| R90 | N-(1-(2-(cyclopropanesulfon- |
| | amido)thiazol-4-yl)-3-methoxypropyl)-4- |
| | (6-ethoxy-pyrazin-2-yl)benzamide |
| | 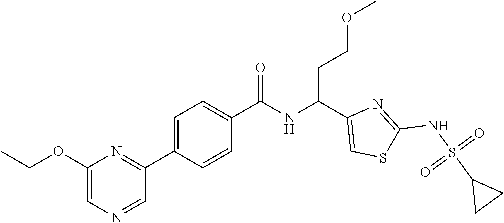 [SMILES: CCOc1cncc(-c2ccc(C(=O)NC(CCOC)c3csc(NS(=O)(=O)C4CC4)n3)cc2)n1] |
| R91 | N-(1-(2-(cyclopropanesulfon- |
| | amido)thiazol-4-yl)ethyl)-4-(6- |
| | ethoxypyrazin-2-yl)-2-fluorobenzamide |
| | 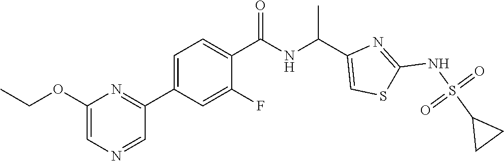 [SMILES: CCOc1cncc(-c2ccc(C(=O)NC(C)c3csc(NS(=O)(=O)C4CC4)n3)c(F)c2)n1] |
| R92 | SINGLE ENANTIOMER - |
| | stereochemistry unassigned |
| | N-(1-(2-(cyclopropanesulfon- |
| | amido)thiazol-4-yl)-3-methoxypropyl)-4- |
| | (6-ethoxypyrazin-2-yl)-2- |
| | fluorobenzamide |
| | 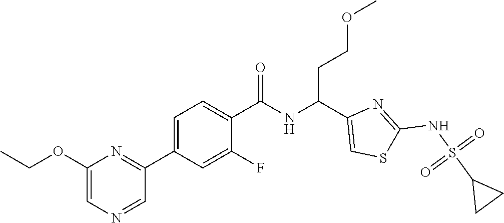 [SMILES: CCOc1cncc(-c2ccc(C(=O)NC(CCOC)c3csc(NS(=O)(=O)C4CC4)n3)c(F)c2)n1] |
| R93 | SINGLE ENANTIOMER - |
| | stereochemistry unassigned |
| | N-(1-(2-(cyclopropanesulfon- |
| | amido)thiazol-4-yl)-3-methoxypropyl)-4- |
| | (6-ethoxypyrazin-2-yl)-2- |
| | fluorobenzamide |
| | 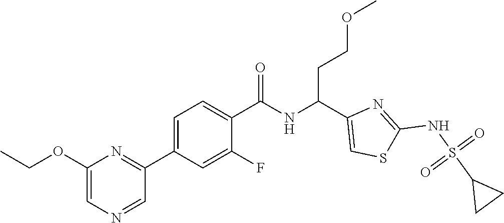 [SMILES: CCOc1cncc(-c2ccc(C(=O)NC(CCOC)c3csc(NS(=O)(=O)C4CC4)n3)c(F)c2)n1] |

N-(4-(2-((4-(6-Ethoxypyrazin-2-yl)-2-fluorobenzyl)amino)propan-2-yl)thiazol-2-yl)cyclopropanesulfonamide R94

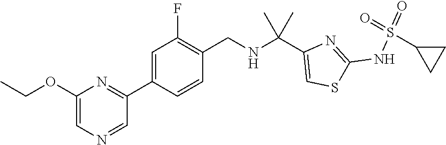 [SMILES: CCOc1cncc(-c2ccc(CNC(C)(C)c3csc(NS(=O)(=O)C4CC4)n3)c(F)c2)n1]

A solution of 4-(6-ethoxypyrazin-2-yl)-2-fluorobenzaldehyde (75 mg, 0.305 mmol) INTF57 and N-(4-(2-aminopropan-2-yl)thiazol-2-yl)cyclopropanesulfonamide INTE14 (80 mg, 0.305 mmol) in DCM (2 mL) was treated with AcOH (0.02 mL, 0.35 mmol) and stirred for 1 hr whereupon sodium tiacetoxyborohydride (70 mg, 0.33 mmol) was added and the reaction mixture was stirred at RT for 4 hrs. The reaction mixture was treated with 1% NH₃ in MeOH (2 mL) and concentrated in vacuo. The crude product was purified by chromatography on RP Flash C18 (12 g cartridge, 15-70% MeCN/10 mM Ammonium Bicarbonate) to N-(4-(2-((4-(6-ethoxypyrazin-2-yl)-2-fluorobenzyl)amino)propan-2-yl)thiazol-2-yl)cyclopropanesulfonamide (30 mg, 0.056 mmol, 18% yield) was isolated as a colourless solid. Rt 0.95 min (UPLC acidic); m/z 492 (M+H)⁺ (ES⁺). ¹H NMR (500 MHz, DMSO-d6) δ 8.86-8.81 (m, 1H), 8.28-8.18 (m, 1H), 7.96-7.93 (m, 1H), 7.91-7.82 (m, 1H), 7.67-7.50 (m, 1H), 6.49-6.14 (v. br. m., 3H), 4.49 (q, J=7.1 Hz, 2H), 3.59-3.48 (m, 2H), 2.50-2.39 (m, 1H), 1.48-1.14 (m, 9H), 0.91-0.59 (m, 4H).
N-(4-(2-(((5-(6-Ethoxypyrazin-2-yl)pyridin-2-yl)methyl)amino)propan-2-yl)thiazol-2-yl)cyclopropanesulfonamide R95

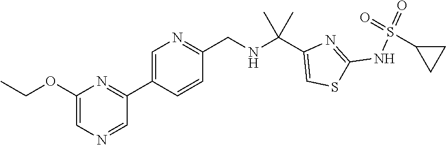 [SMILES: CCOc1cncc(-c2ccc(CNC(C)(C)c3csc(NS(=O)(=O)C4CC4)n3)nc2)n1]

Prepared as for R94 using 5-(6-ethoxypyrazin-2-yl)picolinaldehyde (70 mg, 0.305 mmol) INTF55 and N-(4-(2-aminopropan-2-yl)thiazol-2-yl)cyclopropanesulfonamide INTE14 (80 mg, 0.305 mmol) to afford N-(4-(2-(((5-(6-ethoxypyrazin-2-yl)pyridin-2-yl)methyl)amino)propan-2-yl)thiazol-2-yl)cyclopropanesulfonamide (36 mg, 0.073 mmol, 24% yield) as a red solid. Rt 0.81 min (UPLC acidic); m/z 475 (M+H)⁺ (ES⁺). ¹H NMR (500 MHz, DMSO-d6) δ 9.21 (d, J=2.3 Hz, 1H), 8.88 (s, 1H), 8.44 (dd, J=8.2, 2.4 Hz, 1H), 8.30 (s, 1H), 7.62 (d, J=8.1 Hz, 1H), 6.50 (s, 1H), 4.49 (q, J=7.0 Hz, 2H), 3.68 (s, 2H), 2.59-2.53 (m, 1H), 1.45-1.37 (m, 9H), 0.92-0.82 (m, 4H), 2×N−H not observed.

### BIOLOGICAL EXAMPLES

### Biological Example 1—Human CTPS1 Enzyme Inhibition

The enzyme inhibitory activities of compounds invented against the target of interest were determined using the ADP-Glo™ Max assay (Promega, UK). Assays for human CTPS1 were performed in 1× assay buffer containing 50 mM Tris, 10 mM MgCl₂, 0.01% Tween-20, pH to 8.0 accordingly. Finally, immediately before use, L-cysteine was added to the 1× assay buffer to a final concentration of 2 mM. All reagents are from Sigma-Aldrich unless specified otherwise. Human full length active C-terminal FLAG-His₈-tag CTPS1 (UniProtKB—P17812, CTPS1[1-591]-GGDYKDDDDKGGHHHHHHHH (CTPS1[1-591]—SEQ ID NO: 1)) was obtained from Proteros biostructures GmbH.

Assay Procedure

3× human CTPS1 protein was prepared in 1× assay buffer to the final working protein concentration required for the reaction. A 2 uL volume per well of 3× human CTPS1 protein was mixed with 2 uL per well of 3× test compound (compound prepared in 1× assay buffer to an appropriate final 3× compound concentration respective to the concentration response curve designed for the compounds under test) for 10 minutes at 25° C. The enzymatic reaction was then initiated by addition of a 2 uL per well volume of a pre-mixed substrate mix (UltraPure ATP from ADP-Glo™ Max kit (0.31 mM), GTP (0.034 mM), UTP (0.48 mM) and L-glutamine (0.186 mM)) and the mixture was incubated for an appropriate amount of time within the determined linear phase of the reaction at 25° C. under sealed plate conditions with constant agitation at 500 revolutions per minute (rpm). ADP-Glo™ Max reagent was added for 60 minutes (6 μL per well) and subsequently ADP-Glo™ Max development reagent was added for 60 minutes (12 uL per well) prior to signal detection in a microplate reader (EnVision® Multilabel Reader, Perkin Elmer). Following each reagent addition over the course of the assay, assay plates were pulse centrifuged for 30 seconds at 500 rpm.

In all cases, the enzyme converts ATP to ADP and the ADP-Glo™ Max reagent subsequently depletes any remaining endogenous ATP in the reaction system. The ADP-Glo™ Max detection reagent converts the ADP that has been enzymatically produced back into ATP and using ATP as a substrate together with luciferin for the enzyme luciferase, light is generated which produces a detectable luminescence. The luminescent signal measured is directly proportional to the amount of ADP produced by the enzyme reaction and a reduction in this signal upon compound treatment demonstrates enzyme inhibition. The percentage inhibition produced by each concentration of compound was calculated using the equation shown below:

$$\% Inhibition = 1 - \frac{( {Mean}_{Min} - {Mean}_{Inh} )}{( {Mean}_{Min} - {Mean}_{Max} )} \times 100$$

Percentage inhibition was then plotted against compound concentration, and the 50% inhibitory concentration (IC₅₀) was determined from the resultant concentration-response curve.

The data for compounds of formula (I) tested are presented below.

TABLE 9
Human CTPS1 Enzyme Inhibition data grouped by potency range (±
indicates IC₅₀ in the range of >10 to 20 micromolar, + indicates IC₅₀
in the range >1 to 10 micromolar, ++ indicates IC₅₀ in the
range >0.1 to 1 micromolar, +++ indicates IC₅₀ of ≤0.1 micromolar)

| P | CTPS1 |
|---|---|
| P1 | ++ |
| P2 | +++ |
| P3 | +++ |
| P4 | ++ |
| P5 | + |
| P6 | ++ |
| P7 | ++ |
| P8 | +++ |
| P9 | +++ |
| P10 | +++ |
| P11 | +++ |
| P12 | +++ |
| P13 | ++ |
| P14 | ++ |
| P15 | + |
| P16 | ++ |
| P17 | + |
| P18 | +++ |
| P19 | +++ |
| P20 | +++ |
| P21 | +++ |
| P22 | ++ |
| P23 | ++ |
| P24 | ++ |
| P25 | ++ |
| P26 | ++ |
| P27 | +++ |
| P28 | ++ |
| P29 | + |
| P30 | +++ |
| P31 | +++ |
| P32 | +++ |
| P33 | +++ |
| P34 | +++ |
| P35 | ++ |
| P36 | + |
| P37 | +++ |
| P38 | +++ |
| P39 | +++ |
| P40 | ++ |
| P41 | +++ |
| P42 | + |
| P43 | ++ |
| P44 | ++ |
| P45 | ++ |
| P46 | ++ |
| P47 | +++ |
| P48 | ++ |
| P49 | +++ |
| P50 | + |
| P51 | ++ |
| P52 | ++ |
| P53 | ++ |
| P54 | ++ |
| P55 | +++ |
| P56 | ++ |
| P57 | ++ |
| P58 | ++ |
| P59 | + |
| P60 | + |
| P61 | + |
| P62 | + |
| P63 | ± |
| P64 | +++ |
| P65 | +++ |
| P66 | ++ |
| P67 | +++ |
| P68 | ++ |
| P69 | ++ |
| P70 | + |
| P71 | + |
| P72 | ++ |
| P73 | ++ |
| P74 | ++ |
| P75 | ++ |
| P76 | ++ |
| P77 | + |
| P78 | ++ |
| P79 | ++ |
| P80 | ++ |
| P81 | +++ |
| P82 | + |
| P83 | +++ |
| P84 | ++ |
| P85 | ++ |
| P86 | + |
| P87 | +++ |
| P88 | +++ |
| P89 | +++ |
| P90 | ++ |
| P91 | ++ |
| P92 | ++ |
| P93 | + |
| P94 | +++ |
| P95 | +++ |
| P96 | +++ |
| P97 | +++ |
| P98 | +++ |
| P99 | ++ |
| P100 | ++ |
| P101 | ++ |
| P102 | ++ |
| P103 | ++ |
| P104 | ++ |
| P105 | +++ |
| P106 | +++ |
| P107 | +++ |
| P108 | +++ |
| P109 | ++ |
| P110 | +++ |
| P111 | ++ |
| P112 | +++ |
| P113 | +++ |
| P114 | +++ |
| P115 | +++ |
| P116 | ++ |
| P117 | +++ |
| P118 | +++ |
| P122 | ++ |
| P123 | ++ |
| P124 | ++ |
| P125 | ++ |
| P126 | +++ |
| P128 | ++ |
| P129 | ++ |
| P130 | ++ |
| P131 | ++ |
| P132 | ++ |
| P133 | + |
| P134 | ++ |
| P135 | ++ |
| P136 | +++ |
| P137 | +++ |
| P138 | ++ |
| P139 | ++ |
| | n = 4 |
| | +++ |
| | n = 6 |
| P140 | ++ |
| P141 | ++ |
| P142 | ++ |
| P143 | +++ |
| P144 | +++ |
| P145 | +++ |
| P146 | +++ |
| P147 | ++ |
| P148 | +++ |
| P149 | +++ |
| P150 | +++ |
| P151 | +++ |
| P152 | +++ |
| P153 | +++ |
| P154 | +++ |
| P155 | +++ |
| P156 | +++ |
| P157 | +++ |
| P158 | ++ |
| P159 | +++ |
| P160 | +++ |
| P161 | +++ |
| P162 | +++ |
| P163 | +++ |
| P164 | +++ |
| P165 | +++ |
| P166 | +++ |
| P167 | ++ |
| P168 | ++ |
| P169 | ++ |
| P170 | ++ |
| P171 | + |
| P172 | ++ |
| P173 | +++ |
| P174 | + |
| P175 | + |
| P176 | + |
| P177 | + |
| P178 | + |
| P179 | + |
| P180 | + |
| P181 | + |
| P182 | ++ |
| P183 | ++ |
| P184 | ++ |
| P185 | + |
| P186 | +++ |
| P187 | ++ |
| P188 | +++ |
| P189 | + |
| P190 | ++ |
| P191 | ++ |
| P192 | + |
| P193 | + |
| P194 | 58.6 uM |
| P195 | +++ |
| P196 | +++ |
| P197 | +++ |
| P198 | +++ |
| P199 | ++ |
| P200 | +++ |
| P201 | +++ |
| P202 | +++ |
| P203 | +++ |
| P204 | +++ |
| P205 | +++ |
| P206 | +++ |
| P207 | +++ |
| P208 | +++ |
| P209 | ++ |
| P210 | ++ |
| P211 | ++ |
| P212 | ++ |
| P213 | ++ |
| P214 | ++ |
| P215 | ++ |
| P216 | +++ |
| P217 | ++ |
| P218 | +++ |
| P219 | ++ |
| P220 | ++ |
| P221 | ++ |
| P222 | ++ |
| P223 | ++ |
| P224 | ++ |
| P225 | +++ |

TABLE 10
Human CTPS1 Enzyme Inhibition data grouped by potency range (±
indicates IC₅₀ in the range of >10 to 20 micromolar, + indicates IC₅₀
in the range >1 to 10 micromolar, ++ indicates IC₅₀ in the
range >0.1 to 1 micromolar, +++ indicates IC₅₀ of ≤0.1 micromolar)

| T # | CTPS1 |
|---|---|
| T1 | + |
| T2 | ++ |
| T3 | ++ |
| T4 | + |
| T5 | +++ |
| T6 | ++ |
| T7 | +++ |
| T8 | +++ |
| T9 | +++ |
| T10 | +++ |
| T11 | ++ |
| T12 | ++ |
| T13 | ++ |
| T14 | ++ |
| T15 | ++ |
| T16 | ++ |
| T17 | ++ |
| T18 | +++ |
| T19 | ++ |
| T20 | ++ |
| T21 | + |
| T22 | +++ |
| T23 | ++ |
| T24 | ++ |
| T25 | + |
| T26 | ++ |
| T27 | ++ |
| T28 | ++ |
| T29 | +++ |
| T30 | +++ |
| T31 | + |
| T32 | +++ |
| T33 | +++ |
| T34 | ++ |
| T35 | +++ |
| T36 | ++ |
| T37 | ++ |
| T38 | +++ |
| T39 | ++ |
| T40 | +++ |
| T41 | ++ |
| T42 | +++ |
| T43 | ++ |
| T44 | ++ |
| T45 | ++ |
| T46 | + |
| T47 | +++ |
| T48 | +++ |
| T49 | ++ |
| T50 | +++ |
| T51 | +++ |
| T52 | +++ |
| T53 | + |
| T54 | ++ |
| T55 | +++ |
| T56 | +++ |
| T57 | +++ |
| T58 | +++ |
| T59 | +++ |
| T60 | ++ |
| T61 | +++ |
| T62 | ++ |
| T63 | ++ |
| T64 | ++ |
| T65 | ++ |
| T66 | ++ |
| T67 | ++ |
| T68 | ++ |
| T69 | + |
| T70 | ++ |
| T71 | +++ |
| T72 | +++ |
| T73 | +++ |
| T74 | ++ |
| T75 | +++ |
| T76 | +++ |
| T77 | +++ |
| T78 | +++ |
| T79 | ++ |
| T80 | +++ |
| T81 | +++ |
| T82 | +++ |
| T83 | + |
| T84 | ++ |
| T85 | ++ |
| T86 | + |
| T87 | ++ |
| T88 | ++ |
| T89 | +++ |
| T90 | +++ |
| T91 | +++ |
| T92 | +++ |
| T93 | +++ |
| T94 | +++ |
| T95 | +++ |
| T96 | +++ |
| T97 | ++ |
| T98 | ++ |
| T99 | +++ |
| T100 | ++ |
| T101 | + |
| T102 | +++ |
| T103 | ++ |
| T104 | ++ |
| T105 | + |
| T106 | +++ |
| T107 | +++ |
| T108 | +++ |
| T109 | +++ |
| T110 | ++ |
| T111 | +++ |
| T112 | +++ |
| T113 | + |
| T114 | ++ |
| T115 | +++ |
| T116 | ++ |
| T117 | ++ |
| T118 | +++ |
| T119 | +++ |
| T120 | +++ |
| T121 | +++ |
| T122 | +++ |
| T123 | +++ |
| T124 | +++ |
| T125 | +++ |
| T126 | +++ |
| T127 | +++ |
| T128 | +++ |
| T129 | +++ |
| T130 | +++ |
| T131 | +++ |
| T132 | ++ |
| T133 | +++ |
| T134 | +++ |
| T135 | ++ |
| T136 | +++ |
| T137 | +++ |
| T138 | +++ |
| T139 | +++ |
| T140 | ++ |
| T141 | ++ |
| T142 | +++ |
| T143 | +++ |
| T144 | ++ |
| T145 | +++ |
| T146 | ++ |
| T147 | +++ |
| T148 | ++ |
| T149 | +++ |
| T150 | ++ |
| T151 | ++ |
| T152 | ++ |
| T153 | ++ |
| T154 | ++ |
| T155 | +++ |
| T156 | +++ |
| T157 | +++ |
| T158 | +++ |
| T159 | +++ |
| T160 | +++ |
| T161 | ++ |
| | (n = 2) |
| | + |
| | (n = 6) |
| T163 | ++ |
| T164 | + |
| T169 | ++ |
| T170 | ++ |
| T171 | +++ |
| T172 | +++ |
| T173 | +++ |
| T174 | +++ |
| T175 | +++ |
| T176 | +++ |
| T177 | +++ |
| T178 | + |
| T179 | +++ |
| T180 | +++ |
| T181 | ++ |
| T182 | +++ |
| T183 | +++ |
| T184 | + |
| T185 | +++ |
| | (n = 1) |
| | ++ |
| | (n = 2) |
| T186 | +++ |
| T187 | +++ |
| T188 | ++ |
| T189 | +++ |
| T190 | +++ |
| T191 | +++ |
| T192 | +++ |
| T193 | +++ |
| T194 | +++ |
| T195 | +++ |
| T196 | +++ |
| T197 | +++ |
| T198 | +++ |
| T199 | +++ |
| T200 | +++ |
| T201 | +++ |
| T202 | +++ |
| T203 | +++ |
| T204 | +++ |
| T205 | +++ |
| T206 | +++ |
| T207 | +++ |
| T208 | +++ |
| T209 | +++ |
| T210 | +++ |
| T211 | +++ |
| | (n = 1) |
| | ++ |
| | (n = 2) |
| T212 | +++ |
| T213 | ++ |
| T214 | +++ |
| T215 | +++ |
| T216 | ++ |
| T217 | +++ |
| T218 | ++ |
| T219 | +++ |
| T220 | +++ |
| T221 | +++ |
| T222 | ++ |
| T223 | +++ |
| T224 | +++ |
| T225 | +++ |
| T226 | +++ |
| T227 | ++ |
| T228 | +++ |
| T229 | +++ |
| T230 | +++ |
| T231 | + |
| T232 | ++ |
| T233 | + |
| T234 | + |
| T235 | ++ |
| T236 | ++ |
| T237 | + |
| T238 | + |
| T239 | ++ |
| T240 | ++ |
| T241 | ++ |
| T242 | + |
| T243 | + |
| T244 | ++ |
| T245 | + |
| T246 | ++ |
| T247 | ++ |
| T248 | + |
| T249 | +++ |
| T250 | + |
| T251 | + |
| T252 | +++ |
| T253 | +++ |
| T254 | ++ |
| T255 | + |
| T256 | +++ |
| T257 | ++ |
| T258 | ++ |
| T259 | +++ |
| T260 | + |
| T261 | +++ |
| T262 | +++ |
| T263 | + |
| T264 | ++ |
| T265 | +++ |
| T266 | +++ |
| T267 | +++ |
| T268 | ++ |
| T269 | ++ |
| T270 | ++ |
| T271 | + |
| T272 | +++ |
| T273 | +++ |
| T274 | +++ |
| T275 | +++ |
| T276 | ++ |
| T277 | ++ |
| T278 | +++ |
| T279 | + |
| T280 | + |
| T281 | ++ |
| T282 | ++ |
| T283 | ++ |
| T284 | + |
| T285 | +++ |
| T286 | +++ |
| T287 | +++ |
| T288 | ++ |
| T289 | ++ |
| T290 | +++ |
| T291 | +++ |
| T292 | ++ |
| T293 | ++ |
| T294 | ++ |
| T295 | ++ |
| T296 | ++ |
| T297 | + |
| T298 | +++ |
| T299 | ++ |
| T300 | ++ |
| T301 | +++ |
| T302 | ++ |
| T303 | + |
| T304 | ++ |
| T305 | ++ |
| T306 | ++ |
| T307 | +++ |
| T308 | ++ |
| T309 | + |
| T310 | + |
| T311 | + |
| T312 | ++ |
| T313 | ++ |
| T314 | ++ |
| T315 | ++ |
| T316 | ++ |
| T317 | ++ |
| T318 | + |
| T319 | + |
| T320 | ++ |
| T321 | +++ |
| T322 | ++ |
| T325 | +++ |
| T326 | +++ |
| T327 | +++ |
| T328 | +++ |
| T329 | +++ |
| T330 | +++ |
| T331 | +++ |
| T332 | ++ |
| T333 | + |
| T334 | ++ |
| T335 | ++ |
| T336 | ++ |
| T337 | +++ |
| T338 | + |
| T339 | + |
| T340 | ++ |
| T341 | +++ |
| T342 | +++ |
| T343 | +++ |
| T344 | +++ |
| T345 | +++ |
| T346 | ++ |
| T347 | +++ |
| T348 | ++ |
| T349 | ++ |
| T350 | ++ |
| T351 | ++ |
| | (n = 1) |
| | +++ |
| | (n = 2) |
| T352 | ++ |
| T353 | ++ |
| T354 | ++ |
| T355 | + |
| T356 | +++ |
| T357 | +++ |
| T358 | +++ |
| T359 | +++ |
| T360 | ++ |
| T361 | ++ |
| T362 | +++ |
| T363 | +++ |
| T364 | ++ |
| T365 | +++ |
| T366 | ++ |
| T367 | +++ |
| T368 | +++ |
| T369 | ++ |
| T370 | + |
| T371 | +++ |
| T372 | ++ |
| T373 | +++ |
| T374 | + |
| T375 | ++ |
| T376 | ++ |
| T377 | ++ |
| T378 | + |
| T379 | + |
| T380 | +++ |
| T381 | +++ |
| T382 | +++ |
| T383 | +++ |
| T384 | ++ |
| T385 | +++ |
| T386 | ++ |
| T387 | +++ |
| T388 | +++ |
| T389 | ++ |
| T390 | +++ |
| T391 | +++ |
| T392 | +++ |
| T393 | ++ |
| T394 | +++ |
| T395 | +++ |
| T396 | ++ |
| T397 | ++ |
| T398 | +++ |
| T399 | +++ |
| T400 | +++ |
| T401 | ++ |
| T402 | + |
| T403 | ++ |
| T404 | ++ |
| T405 | +++ |
| T406 | ++ |
| T407 | +++ |
| T408 | +++ |
| T409 | +++ |
| T410 | +++ |
| T411 | +++ |
| T412 | +++ |
| T413 | +++ |
| T414 | +++ |
| T415 | ++ |
| T416 | +++ |
| T417 | ++ |
| T418 | ++ |
| T419 | +++ |
| T420 | +++ |
| T421 | +++ |
| T422 | ++ |
| T423 | +++ |
| T424 | +++ |
| T425 | +++ |
| T426 | ++ |
| T427 | ++ |
| T428 | ++ |
| T429 | +++ |
| T430 | ++ |
| T431 | +++ |
| T432 | ++ |
| T433 | +++ |
| T434 | +++ |
| T435 | +++ |
| T436 | +++ |
| T437 | +++ |
| T438 | +++ |
| T439 | +++ |
| T440 | +++ |
| T441 | +++ |
| T442 | +++ |
| T443 | +++ |
| T444 | +++ |
| T445 | +++ |
| T446 | +++ |
| T447 | +++ |
| T448 | +++ |
| T449 | +++ |
| T450 | +++ |
| T451 | +++ |
| T452 | +++ |
| T453 | +++ |
| T454 | +++ |
| T455 | +++ |
| T456 | +++ |
| T457 | +++ |
| T458 | +++ |
| T459 | +++ |
| T463 | ++ |
| T464 | +++ |
| T465 | + |

TABLE 11
Human CTPS1 Enzyme Inhibition data grouped by potency range (±
indicates IC₅₀ in the range of >10 to 21 micromolar, + indicates IC₅₀
in the range >1 to 10 micromolar, ++ indicates IC₅₀ in the
range >0.1 to 1 micromolar, +++ indicates IC₅₀ of ≤0.1 micromolar)

| R | CTPS1 |
|---|---|
| R1 | ++ |
| R2 | ++ |
| R3 | ++ |
| R4 | +++ |
| R5 | +++ |
| R6 | +++ |
| R7 | ++ |
| R8 | + |
| R9 | + |
| R10 | +++ |
| R11 | +++ |
| R12 | +++ |
| R13 | +++ |
| R14 | +++ |
| R15 | +++ |
| R16 | ++ |
| R17 | ++ |
| R18 | ++ |
| R19 | +++ |
| R20 | +++ |
| R21 | +++ |
| R22 | ++ |
| R23 | ++ |
| R24 | ++ |
| R25 | ++ |
| R26 | ++ |
| R27 | ± |
| R28 | +++ |
| R29 | +++ |
| R30 | ++ |
| R31 | ++ |
| R32 | ++ |
| R33 | + |
| R34 | + |
| R35 | +++ |
| R36 | ++ |
| R37 | +++ |
| R38 | ++ |
| R39 | ++ |
| R40 | ++ |
| R41 | ++ |
| R42 | +++ |
| R43 | +++ |
| R44 | ++ |
| R45 | ++ |
| R46 | ++ |
| R47 | ++ |
| R48 | ++ |
| R49 | ++ |
| R50 | + |
| R51 | +++ |
| R52 | +++ |
| R53 | ++ |
| R54 | +++ |
| R55 | +++ |
| R56 | +++ |
| R57 | ++ |
| R58 | ++ |
| R59 | ++ |
| R60 | ++ |
| R61 | ++ |
| R62 | ++ |
| R63 | ++ |
| R64 | ++ |
| R65 | +++ |
| R66 | ++ |
| R67 | ++ |
| R68 | + |
| R69 | +++ |
| R70 | ++ |
| R71 | +++ |
| R72 | + |
| R73 | + |
| R74 | ++ |
| R75 | ++ |
| R76 | ++ |
| R77 | + |
| R78 | ++ |
| R79 | ++ |
| R80 | +++ |
| R81 | ++ |
| R82 | +++ |
| R83 | +++ |
| R84 | +++ |
| R85 | ++ |
| R86 | ++ |
| R87 | ++ |
| R88 | ++ |
| R89 | +++ |
| R90 | +++ |
| R91 | +++ |
| R92 | +++ |
| R93 | +++ |

All compounds disclosed in Tables 9 to 11 were found to demonstrate inhibition of CTPS1 enzyme in this assay. Consequently, these compounds may be expected to have utility in the inhibition of CTPS1. The compounds of the invention are also expected to have utility as research tools, for example, for use in CTPS assays.

The data for all compounds of formula (I) (including (I-a), (I-b) and (I-c) tested wherein R₁ is R₁ₐ; and/or R₄ and R₅ are R₄ₐ and R₅ₐ; and/or A is Aₐ are presented below.

TABLE 12
Human CTPS1 Enzyme Inhibition data grouped by potency range (±
indicates IC₅₀ in the range of >10 to 20 micromolar, + indicates IC₅₀
in the range >1 to 10 micromolar, ++ indicates IC₅₀ in the
range >0.1 to 1 micromolar, +++ indicates IC₅₀ of ≤0.1 micromolar)

| P | CTPS1 |
|---|---|
| P226 | +++ |
| P227 | +++ |
| P228 | +++ |
| P229 | +++ |
| P230 | + |
| P235 | ++ |
| P242 | +++ |
| P244 | +++ |
| P248 | ++ |
| P251 | +++ |
| P254 | ++ |
| P255 | ++ |
| P256 | ++ |
| P258 | +++ |
| P260 | +++ |
| P261 | ++ |
| P288 | +++ |
| P289 | +++ |
| P290 | +++ |
| P291 | +++ |
| P292 | +++ |
| P293 | +++ |
| P294 | +++ |
| P295 | +++ |
| P296 | +++ |
| P297 | +++ |
| P298 | +++ |
| P299 | +++ |
| P300 | +++ |
| P301 | +++ |
| P302 | +++ |
| P303 | ++ |
| P304 | +++ |
| P305 | ++ |
| P306 | +++ |
| P307 | +++ |
| P308 | +++ |
| P309 | ++ |
| P310 | +++ |
| P311 | ++ |
| P312 | +++ |
| P313 | +++ |
| P314 | +++ |
| P315 | +++ |
| P316 | +++ |
| P317 | +++ |
| P318 | +++ |

TABLE 13
Human CTPS1 Enzyme Inhibition data grouped by potency range (+++
indicates IC₅₀ of ≤0.1 micromolar)

| R | CTPS1 |
|---|---|
| T466 | ± |

TABLE 14
Human CTPS1 Enzyme Inhibition data grouped by potency range (+++
indicates IC₅₀ of ≤0.1 micromolar)

| R | CTPS1 |
|---|---|
| R94 | +++ |

All compounds of the invention which have been tested were found to demonstrate inhibition of CTPS1 enzyme in this assay (see Tables 12 to 14). Consequently, these compounds may be expected to have utility in the inhibition of CTPS1.

In particular, activity is retained following reduction of the amide (group A) to the amine derivative (see Examples R94 and T466).

### Biological Example 2—RapidFire/MS-Based Enzyme Selectivity Assays

Human CTPS1 Versus CTPS2 Selectivity Assessment by RapidFire/MS Analysis.

The enzyme inhibitory activities against each target isoform of interest may be determined for the compounds of the invention using an optimised RapidFire high-throughput mass spectrometry (RF/MS) assay format. RF/MS assays for both human CTPS1 and CTPS2 may be performed in assay buffer consisting of 50 mM HEPES (Merck), 20 mM MgCl₂, 5 mM KCl, 1 mM DTT, 0.01% Tween-20, pH to 8.0 accordingly. Human full-length active C-terminal FLAG-His-tag CTPS1 (UniProtKB—P17812, CTPS1[1-591]-GGDYKDDDDKGGHHHHHHHH (CTPS1[1-591]-SEQ ID NO: 1)) may be obtained from Proteros biostructures GmbH. Human full length active C-terminal FLAG-His-Avi tagged CTPS2 (UniProtKB—Q9NRF8, CTPS2 [1-586]-DYKDDDDKHHHHHHGLNDIFEAQKIEWHE (CTPS2 [1-586]—SEQ ID NO: 2)) may be obtained from Harker Bio.

Assay Procedure

Human CTPS (1 or 2) protein may be prepared in 1× assay buffer to the final working protein concentration required for the reaction. A 2 uL volume per well of 2×CTPS (1 or 2) protein may be mixed with 40 nL of compound using acoustic (ECHO) delivery and incubated for 10 minutes at 25° C. Each isoform enzymatic reaction may be subsequently initiated by addition of 2 uL per well of a 2× substrate mix in assay buffer. For hCTPS1: ATP (0.3 mM), UTP (0.2 mM), GTP (0.07 mM) and L-glutamine (0.1 mM). For hCTPS2: ATP (0.1 mM), UTP (0.04 mM), GTP (0.03 mM) and L-glutamine (0.1 mM). Each mixture may be incubated for an appropriate amount of time per isoform within the determined linear phase of the reaction at 25° C. A 80 uL volume of stop solution (1% formic acid with 0.5 uM ¹³C₉-¹⁵N₃-CTP in H₂O) may be added and the plate immediately heat-sealed and centrifuged for 10 minutes at 4,000 rpm. Following centrifugation, plates may be loaded onto the Agilent RapidFire microfluidic solid phase extraction system coupled to an API4000 triple quadrupole mass spectrometer (RF/MS) for analysis.

In all cases, the enzyme converts UTP to CTP. Highly specific and sensitive multiple reaction monitoring (MRM) MS methods may be optimised for the detection of the enzymatic reaction product, CTP, and the stable isotope labelled product standard ¹³C₉-¹⁵N₃-CTP. Readout for data analysis may be calculated as the ratio between the peak area of the product CTP and the internal standard ¹³C₉-¹⁵N₃-CTP. For data reporting, the following equation may be used:

$$R = \frac{P}{IS}$$

(R=ratio/readout, P=product signal area, IS=internal standard signal area)

For each screening plate, the means of the negative (DMSO) and positive control values were used for the calculation of the respective assay window (S/B) and Z′ values. The median of the respective control values was used for calculation of percent inhibition according to the following equation:

$$I = \frac{{R}_{neg} - {R}_{sample \%}}{[ {R}_{neg} - {R}_{pos} ]}$$

(I=Inhibition, R_{neq}=median of negative control readout values, Rₚₒₛ=median of positive control readout values, Rₛₐₘₚₗₑ=sample readout value)

Percentage inhibition was then plotted against compound concentration, and the 50% inhibitory concentration (IC₅₀) was determined from the resultant concentration-response curve.

Fold selectivity between CTPS1 and CTPS2 was subsequently calculated according to the following equation:

$$Fold selectivity = \frac{CTPS 2 \vert {C}_{50}}{CTPS 1 \vert {C}_{50}}$$

The data for all compounds disclosed herein that were tested in Biological Example 2 are presented below.

TABLE 15
Selectivity data split into grouping of 2-30 fold (+), >30-60
fold (++) or >60 fold (+++)

| P | Selectivity |
|---|---|
| P1 | + |
| P2 | +++ |
| P9 | +++ |
| P12 | ++ |
| P16 | ++ |
| P18 | ++ |
| P21 | ++ |
| P31 | +++ |
| P34 | + |
| P38 | + |
| P39 | + |
| P59 | + |
| P65 | ++ |
| P68 | ++ |
| P70 | + |
| P74 | ++ |
| P76 | ++ |
| P83 | +++ |
| P87 | ++ |
| P88 | +++ |
| P89 | +++ |
| P95 | + |
| P96 | + |
| P98 | +++ |
| P103 | + |
| P105 | ++ |
| P108 | +++ |
| P110 | ++ |
| P112 | ++ |
| P113 | + |
| P114 | +++ |
| P115 | +++ |
| P118 | +++ |
| P125 | ++ |
| P128 | + |
| P132 | ++ |
| P136 | +++ |
| P143 | +++ |
| P145 | +++ |
| P146 | +++ |
| P151 | +++ |
| P155 | + |
| P158 | + |
| P159 | +++ |
| P161 | + |
| P162 | ++ |
| P163 | +++ |
| P164 | + |
| P188 | ++ |
| P191 | ++ |
| P195 | +++ |
| P196 | +++ |
| P197 | +++ |
| P198 | +++ |
| P200 | ++ |
| P201 | +++ |
| P202 | +++ |
| P205a | +++ |
| P205b | ++ |
| P206 | +++ |
| P207 | + |
| P216 | +++ |
| P221 | + |
| P222 | + |

TABLE 16
Selectivity data split into grouping of 2-30 fold (+), >30-60
fold (++) or >60 fold (+++)

| R | Selectivity |
|---|---|
| R5 | + |
| R6 | ++ |
| R7 | ++ |
| R11 | ++ |
| R19 | ++ |
| R23 | ++ |
| R25 | ++ |
| R26 | +++ |
| R41 | ++ |
| R42 | ++ |
| R43 | ++ |
| R45 | ++ |
| R46 | + |
| R47 | + |
| R48 | ++ |
| R51 | ++ |
| R52 | +++ |
| R54 | + |
| R55 | +++ |
| R56 | +++ |
| R62 | +++ |
| R63 | + |
| R64 | ++ |
| R68 | ++ |
| R69 | +++ |
| R70 | +++ |
| R71 | +++ |
| R73 | + |
| R74 | ++ |
| R75 | +++ |
| R76 | + |
| R78 | ++ |
| R79 | + |
| R80 | +++ |
| R82 | +++ |
| R83 | +++ |
| R84 | +++ |
| R86 | +++ |
| R87 | ++ |
| R88 | +++ |
| R89 | +++ |
| R90 | ++ |
| R91 | +++ |
| R92 | +++ |
| R93 | +++ |

The data for all compounds of formula (I) tested wherein R₁ is R₁ₐ; and/or R₄ and R₅ are R₄ₐ and R₅ₐ; and/or A is Aₐ are presented in Table 17.

TABLE 17
Selectivity data split into grouping of 2-30 fold (+), >30-60
fold (++) or >60 fold (+++)

| P | Selectivity |
|---|---|
| P226 | +++ |
| P227 | +++ |
| P228 | +++ |
| P229 | +++ |
| P230 | + |
| P242 | +++ |
| P244 | +++ |
| P248 | +++ |
| P251 | +++ |
| P289 | +++ |
| P290 | +++ |
| P291 | +++ |
| P292 | +++ |
| P293 | +++ |
| P294 | +++ |
| P297 | +++ |
| P298 | +++ |
| P299 | +++ |
| P300 | +++ |
| P301 | +++ |
| P302 | +++ |
| P303 | +++ |
| P305 | +++ |
| P306 | +++ |
| P307 | +++ |
| P308 | +++ |
| P309 | +++ |
| P312 | +++ |
| P313 | +++ |
| P314 | +++ |
| P315 | +++ |
| P316 | +++ |
| P317 | +++ |
| P318 | +++ |

All compounds tested in the assay described in Biological Assay 2 were found to have at least 2 fold selectivity for CTPS1 over CTPS2, with many compounds having a selectivity for CTPS1 of over 60 fold. In particular, these compounds may be expected to have utility in the treatment of diseases whereby a selective CTPS1 compound is beneficial.

The compounds of the invention are also expected to have utility as research tools, for example, for use in CTPS assays.

Throughout the specification and the claims and clauses which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the claims which follow.

All publications, including but not limited to patents and patent applications, cited in this specification are herein incorporated by reference as if each individual publication were specifically and individually indicated to be incorporated by reference herein as though fully set forth.

### CLAUSES OF THE INVENTION

Clause 1. A compound of formula (I):

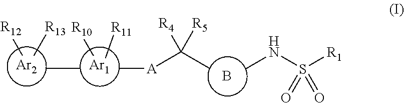

wherein ring B is selected from the group consisting of:

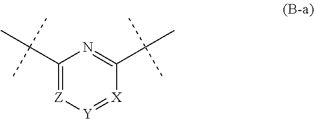

wherein X, Y and Z are as defined below; and

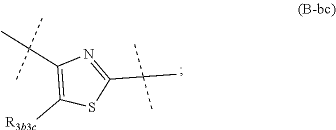

wherein R_{3b3c} is R_{3b} or R_{3c} as defined below;

wherein when B is (B-a) the compound of formula (I) is a compound of formula (I-a):

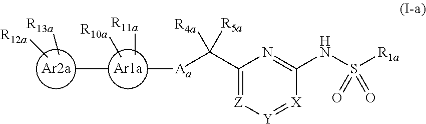

wherein:
Aₐ is Aₐₐ or A_{ba};
   wherein:
   Aₐₐ is an amine linker having the following structure: —NH—, —CH₂NH— or —NHCH₂—;
   A_{ba} is an amide linker having the following structure: —C(═O)NH— or —NHC(═O)—;
X is N or CH;
Y is N or CR₂ₐ;
Z is N or CR₃ₐ;
   with the proviso that when at least one of X or Z is N, Y cannot be N;
R₂ₐ is H, halo, C₁₋₂alkyl, OC₁₋₂alkyl, C₁₋₂haloalkyl or OC₁₋₂haloalkyl; and
R₃ₐ is H, halo, CH₃, OCH₃, CF₃ or OCF₃;
   wherein at least one of R₂ₐ and R₃ₐ is H;
R₁ₐ is R₁ₐₐ or R_{1ba};
   wherein:
   R₁ₐₐ is NR₃₂ₐR₃₃ₐ;
   R_{1ba} is C₁₋₅alkyl, C₀₋₂alkyleneC₃₋₅cycloalkyl which cycloalkyl is optionally substituted by CH₃, or CF₃;
R₄ₐ and R₅ₐ are R₄ₐₐ and R₅ₐₐ, or R_{4ba} and R_{5ba};
   wherein:
   R₄ₐₐ and R₅ₐₐ together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkykeneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR₂₁ₐR₂₂ₐ; or
      one of the carbons of the C₃₋₆ cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl; or
   R₄ₐₐ and R₅ₐₐ together with the carbon atom to which they are attached form a C₃₋₆heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl; or R₄ₐₐ and R₅ₐₐ together with the carbon atom to which they are attached form a C₃₋₆heterocycloalkyl comprising one nitrogen atom, wherein said nitrogen atom is substituted by —S(O)₂R₂₉ₐ; or
   R_{4ba} and R_{5ba} are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R_{4ba} and R_{5ba} together with the carbon atom to which they are attached form a C₃₋₆cycloalkyl or C₃₋₆heterocycloalkyl; and
   when Aₐ is —NHC(═O)— or —NHCH₂—:
   R_{4ba} and R_{5ba} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and NR₂₁ₐR₂₂ₐ;
Ar1a is a 6-membered aryl or heteroaryl;
Ar2a is a 6-membered aryl or heteroaryl and is attached to Ar1a in the para position relative to group Aₐ;
R₁₀ₐ is H, halo, C₁₋₃alkyl, C₁₋₂haloalkyl, OC₁₋₂alkyl, OC₁₋₂haloalkyl or CN;
R₁₁ₐ is H, F, Cl, C₁₋₂alkyl, CF₃, OCH₃ or CN;
R₁₂ₐ is attached to Ar2 in the ortho or meta position relative to Ar1a and R₁₂ₐ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃ₐR₂₄ₐ; and
   when Aₐ is —NHC(═O)—, —NH— or —NHCH₂—:
   R₁₂ₐ may additionally be selected from CN, OCH₂CH₂N(CH₃)₂ and a C₃₋₆heterocycloalkyl comprising one nitrogen located at the point of attachment to Ar2a, or R₁₂ₐ together with a nitrogen atom to which it is attached forms an N-oxide (N⁺—O—);
R₁₃ₐ is H or halo;
R₂₁ₐ is H, C₁₋₅alkyl, C(O)C₁₋₅alkyl, C(O)OC₁₋₅alkyl, C₁₋₃alkylOC₁₋₂alkyl, C₁₋₄haloalkyl, or C₄₋₆heterocycloalkyl;
R₂₂ₐ is H or CH₃;
R₂₃ₐ is H or C₁₋₂alkyl; and
R₂₄ₐ is H or C₁₋₂alkyl
R₂₉ₐ is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl;
R₃₂ₐ is C₁₋₃alkyl and R₃₃ is C₁₋₃alkyl; or
R₃₂ₐ and R₃₃ₐ together with the nitrogen atom to which they are attached form a C₃₋₅ heterocycloalkyl;
wherein:
R₁ₐ is R₁ₐₐ; and/or
R₄ₐ and R₅ₐ are R₄ₐₐ and R₅ₐₐ; and/or
Aₐ is Aₐₐ; and

wherein when B is (B-bc) and R_{3a3c} is R_{3b}, the compound of formula (I) is a compound of formula (I-b):

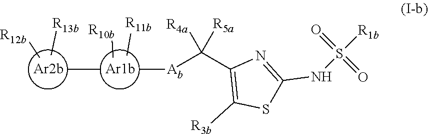

A_{b} is A_{ab} or A_{bb};
wherein:
   A_{ab} is —NR_{6b}CH₂— or —NR_{6b}—;
   A_{bb} is —NR_{6b}C(═O)—;
R_{1b} is R_{1ab} or R_{1bb};
wherein:
   R_{1ab} is NR_{32b}R_{33b};
   R_{1bb} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R_{3b} is H, halo, CH₃, OC₁₋₂alkyl or CF₃;
or R_{3b} together with R_{5bb} forms a 5- or 6-membered cycloalkyl or 5 or 6 membered oxygen-containing heterocycloalkyl;
R_{4b} and R_{5b} are either R_{4ab} and R_{5ab} or R_{4bb} and R_{5bb};
wherein:
   R_{4ab} and R_{5ab} together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21b}R₂₂ₐ; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ab} and R_{5ab} together with the carbon atom to which they are attached may be substituted by one or two substituents, each substituent being independently selected from the group consisting of C₁₋₃ alkyl or OC₁₋₃alkyl; or
   R_{4ab} and R_{5ab} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heteroycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl; or
   R_{4ab} and R_{5ab} together with the carbon atom to which they are attached form a C₃₋₆heterocycloalkyl comprising one nitrogen atom, wherein said nitrogen atom is substituted by —S(O)₂R_{29b}; or
   R_{4bb} and R_{5bb} are each independently H, halo, C₁₋₆alkyl, C₀₋₂alkyleneC₃₋₆cycloalkyl, C₀₋₂alkyleneC₃₋₆heterocycloalkyl, OC₁₋₆alkyl, OC₀₋₂alkyleneC₃₋₆cycloalkyl, C₁₋₃alkyleneOC₁₋₃alkyl, C₁₋₆alkylOH, C₁₋₆haloalkyl, OC₁₋₆haloalkyl or NR_{21b}R_{22b},
      or R_{4bb} is H and R_{5bb} together with R_{3b} form a 5- or 6-membered cycloalkyl or 5 or 6 membered oxygen-containing heterocycloalkyl,
      or R_{4bb} and R_{5bb} together with the carbon atom to which they are attached form a C₃₋₆cycloalkyl or C₃₋₆heterocycloalkyl,
      or R_{4bb} is H and R_{5bb} and R_{6b} are a C₂₋₃alkylene chain forming a 5- or 6-membered ring;
      or R_{4bb} is O and R_{5bb} is absent;
R_{6b} is H or C₁₋₃alkyl,
   or R_{6b} together with R_{11b} when in the ortho-position to group A_{b} are a C₂alkylene chain forming a 5-membered ring,
   or R_{5bb} and R_{6b} are a C₂₋₃alkylene chain forming a 5- or 6-membered ring and R_{4bb} is H;
Ar1b is 6-membered aryl or heteroaryl;
Ar2b is a 6-membered aryl or heteroaryl and is attached to Ar1b in the pare position relative to group A_{b};
R_{10b} is H, halo, C₁₋₃alkyl, OC₁₋₂alkyl, C₁₋₂haloalkyl, OC₁₋₂haloalkyl or CN;
R_{11b} is H, F, Cl, CH₃, ethyl, OCH₃, CF₃, OCF₃ or CN,
   or R_{11b}, when in the ortho-position to group A_{b}, together with R_{6b} are a C₂alkylene chain forming a 5-membered ring;
R_{12b} is attached to Ar2b in the ortho or meta position relative to Ar1b and R_{12b} is H, halo, C₁₋₄alkyl, C₂₋₄alkynyl, C₀₋₂alkyleneC₃₋₅cycloalkyl, OC₁₋₄alkyl, OC₀₋₂alkyleneC₃₋₅cycloalkyl, OCH₂CH₂N(CH₃)₂, OH, C₁₋₄alkylOH, CN, C₁₋₃alkyleneOC₁₋₃alkyl, C₁₋₄haloalkyl, OC₁₋₄haloalkyl, C(═O)C₁₋₂alkyl, NR_{23b}R_{24b}, SO₂C₁₋₄alkyl, SOC₁₋₄alkyl, SC₁₋₄alkyl, SH, C(O)N(CH₃)₂, NHC(O)C₁₋₃alkyl, C₃₋₆heterocycloalkyl comprising one nitrogen located at the point of attachment to Ar2b, or R_{12b} together with a nitrogen atom to which it is attached forms an N-oxide (N⁺—O⁻);
R_{13b} is H, halo, CH₃ or OCH₃;
R_{21b} is H, C₁₋₅alkyl, C(O)C₁₋₅alkyl, C(O)OC₁₋₅alkyl, C₁₋₃alkylOC₁₋₂alkyl, C₁₋₄haloalkyl, or C₄₋₆heterocycloalkyl;
R₂₂ₐ is H or CH₃;
R_{22b} is H or C₁₋₂alkyl;
R_{24b} is H or C₁₋₂alkyl;
R_{29b} is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl; and
R_{32b} is C₁₋₃alkyl and R_{33b} is C₁₋₃alkyl; or
R_{32b} and R_{33b} together with the nitrogen atom to which they are attached form a C₃₋₅heterocycloalkyl;
wherein:
R_{1b} is R_{1ab}; and/or
R_{4b} and R_{5b} are R_{4ab} and R_{5ab}; and/or
A is A_{ab}; or

wherein when B is (B-bc) and R_{3b3c} is R_{3c}, the compound of formula (I) is a compound of formula (I-c):

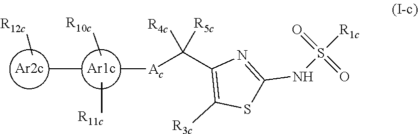

wherein:
A_{c} is A_{ac} or A_{bc};
   wherein:
   A_{ac} is —CH₂NR_{6c}—;
   A_{bc} is —C(═O)NR_{6c}—;
R_{1c} is R_{1ac} or R_{1bc};
wherein:
   R_{1ac} is NR_{32c}R_{33c};
R_{1bc} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R_{3c} is H, CH₃, halo, OC₁₋₂alkyl or CF₃;
R_{4c} and R_{5c} are either R_{4ac} and R_{5bc} or R_{4bc} and R_{5bc};
wherein:
   R_{4ac} and R_{5ac} together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21c}R_{22c}; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ac} and R_{5ac} together with the carbon atom to which they are attached may be substituted by one or two substituents, each substituent being independently selected from the group consisting of C₁₋₃ alkyl or OC₁₋₃alkyl; or
   R_{4ac} and R_{5ac} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heteroycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl; or
   R_{4ac} and R_{5ac} together with the carbon atom to which they are attached form a C₃₋₆heterocycloalkyl comprising one nitrogen atom, wherein said nitrogen atom is substituted by —S(O)₂R_{29c}; or
   R_{4bc} and R_{5bc} are each independently H, C₁₋₆alkyl, C₀₋₂alkyleneC₃₋₆cycloalkyl, C₀₋₂alkyleneC₃₋₆heterocycloalkyl, C₁₋₃alkyleneOC₁₋₃alkyl, C₁₋₆alkylOH or C₁₋₆haloalkyl,
   or R_{4bc} and R_{5bc} together with the carbon atom to which they are attached form a C₃₋₆cycloalkyl or C₃₋₆heterocycloalkyl ring;
R_{6c} is H or C₁₋₃alkyl;
Ar1c is a 6-membered aryl or heteroaryl;
Ar2c is a 6-membered aryl or heteroaryl and is attached to Ar1c in the para position relative to group A_{c};
R_{10c} is H, halo, C₁₋₃alkyl, OC₁₋₂alkyl, C₁₋₂haloalkyl, OC₁₋₂haloalkyl or CN;
R_{11c} is H, F, Cl, CH₃, ethyl, OCH₃, CF₃, OCF₃ or CN;
R_{12c} is attached to Ar2c in the meta or ortho position relative to Ar1c and R_{12c} is H, halo, C₁₋₄alkyl, C₂₋₄alkynyl, C(═O)C₁₋₂alkyl, C₀₋₂alkyleneC₃₋₅cycloalkyl, OC₁₋₄alkyl, C₁₋₃ alkyleneOC₁₋₃alkyl, C₁₋₄haloalkyl, OC₁₋₄haloalkyl, CN, OC₀₋₂alkyleneC₃₋₅cycloalkyl, OCH₂CH₂N(CH₃)₂, OH, C₁₋₄alkylOH, NR_{23c}R_{24c}, SO₂CH₃, C(O)N(CH₃)₂, NHC(O)C₁₋₃alkyl, or a C₃₋₆heterocycloalkyl comprising one nitrogen located at the point of attachment to Ar2c, or R_{12c} together with a nitrogen atom to which it is attached forms an N-oxide (N⁺—O⁻);
R_{21c} is H, C₁₋₅alkyl, C(O)C₁₋₅alkyl, C(O)OC₁₋₅alkyl, C₁₋₃alkylOC₁₋₂alkyl, C₁₋₄haloalkyl, or C₄₋₆heterocycloalkyl;
R_{22c} is H or CH₃;
R_{23c} is H or C₁₋₂alkyl;
R_{24c} is H or C₁₋₂alkyl;
R_{29c} is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl; and
R_{32c} is C₁₋₃alkyl and R_{33c} is C₁₋₃alkyl; or
R_{32c} and R_{33c} together with the nitrogen atom to which they are attached form a C₃₋₅heterocycloalkyl;

wherein:
R_{1c} is R_{1ac}; and/or
R_{4c} and R_{5c} are R_{4ac} and R_{5ac}; and/or
A_{c} is A_{ac};

or a salt and/or solvate thereof and/or derivative thereof.

Clause 2. A compound of formula (I) according to clause 1 wherein the compound of formula (I) is a compound of formula (I-a).

Clause 3. A compound of formula (I) according to clause 1 or clause 2 wherein the compound of formula (I) is a compound of formula (I-a):

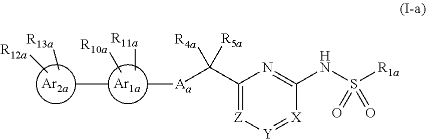

wherein
Aₐ is Aₐₐ or A_{ba};
wherein
   Aₐₐ is an amine linker having the following structure: —NH—, —CH₂NH— or —NHCH₂—;
   A_{ba} is an amide linker having the following structure: —C(═O)NH— or —NHC(═O)—;
X is N or CH;
Y is N or CR₂ₐ;
Z is N or CR₃ₐ;
   with the proviso that when at least one of X or Z is N, Y cannot be N;
R₁ₐ is R₁ₐₐ or R_{1ba};
   wherein
   R₁ₐₐ is NR₃₂ₐR₃₃ₐ;
   R_{1ba} is C₁₋₅alkyl, C₀₋₂alkyleneC₃₋₅cycloalkyl which cycloalkyl is optionally substituted by CH₃, or CF₃;
R₂ₐ is H, halo, C₁₋₂alkyl, OC₁₋₂alkyl, C₁₋₂haloalkyl or OC₁₋₂haloalkyl;
R₃ₐ is H, halo, CH₃, OCH₃, CF₃ or OCF₃;
   wherein at least one of R₂ₐ and R₃ₐ is H;
R₄ₐ and R₅ₐ are R₄ₐₐ and R₅ₐₐ, or R_{4ba} and R_{5ba};
   wherein
   R₄ₐₐ and R₅ₐₐ together with the carbon atom to which they are attached form a C₃₋₆cycloalkyl which is:
      substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR₂₁ₐR₂₂ₐ; or
      one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl; or
   R₄ₐₐ and R₅ₐₐ together with the carbon atom to which they are attached form a C₃₋₆heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl; or
   R₄ₐₐ and R₅ₐₐ together with the carbon atom to which they are attached form a C₃₋₆heterocycloalkyl comprising one nitrogen atom, wherein said nitrogen atom is substituted by —S(O)₂R₂₉ₐ; or
R_{4ba} and R_{5ba} are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R₄ₐ and R₅ₐ together with the carbon atom to which they are attached form a C₃₋₆cycloalkyl or C₃₋₆heterocycloalkyl; and
   when Aₐ is —NHC(═O)— or —NHCH₂—:
   R_{4ba} and R_{5ba} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₆alkyl and NR₂₁ₐR₂₂ₐ;
Ar1a is a 6-membered aryl or heteroaryl;
Ar2a is a 6-membered aryl or heteroaryl and is attached to Ar1a in the para position relative to group Aₐ;
R₁₀ₐ is H, halo, C₁₋₃alkyl, C₁₋₂haloalkyl, OC₁₋₂alkyl, OC₁₋₂haloalkyl or CN;
R₁₁ₐ is H, F, Cl, C₁₋₂alkyl, CF₃, OCH₃ or CN;
R₁₂ₐ is attached to Ar2a in the ortho or meta position relative to Ar1a and R₁₂ₐ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃ₐR₂₄ₐ; and
   when Aₐ is —NHC(═O)—, —NH— or —NHCH₂—:
   R₁₂ₐ may additionally be selected from CN, OCH₂CH₂N(CH₃)₂ and a C₃₋₆heterocycloalkyl comprising one nitrogen located at the point of attachment to Ar2a, or R₁₂ₐ together with a nitrogen atom to which it is attached forms an N-oxide (N⁺—O⁻);
R₁₃ₐ is H or halo;
R₂₁ₐ is H, C₁₋₅alkyl, C(O)C₁₋₅alkyl, C(O)OC₁₋₅alkyl;
R₂₂ₐ is H or CH₃;
R₂₃ₐ is H or C₁₋₂alkyl; and
R₂₄ₐ is H or C₁₋₂alkyl;
R₂₉ₐ is C₁₋₃alkyl, C₀₋₂alkyleneC₃₋₅-cycloalkyl which cycloalkyl is optionally substituted by CH₃, or CF₃;
R₃₂ₐ is C₁₋₃alkyl and R₃₃ₐ is C₁₋₃alkyl; or
R₃₂ₐ and R₃₃ₐ together with the nitrogen atom to which they are attached form a C₃₋₅heterocycloalkyl;

wherein
R₁ₐ is R₁ₐₐ; and/or
R₄ₐ and R₅ₐ are R₄ₐₐ and R₅ₐₐ; and/or
A is Aₐ;

or a salt and/or solvate thereof and/or derivative thereof.

Clause 4. The compound according to any one of clauses 1 to 3 wherein Aₐ is —C(═O)NH—.

Clause 5. The compound according to any one of clauses 1 to 3 wherein Aₐ is —NHC(═O)—.

Clause 6. The compound according to any one of clauses 1 to 3 wherein Aₐ is —NH—.

Clause 7. The compound according to any one of clauses 1 to 3 wherein Aₐ is —CH₂NH—.

Clause 8. The compound according to any one of clauses 1 to 3 wherein Aₐ is —NHCH₂—.

Clause 9. The compound according to any one of clauses 1 to 8 wherein X is N.

Clause 10. The compound according to any one of clauses 1 to 8 wherein X is CH.

Clause 11. The compound according to any one of clauses 1 to 8 or 10 wherein Y is N.

Clause 12. The compound according to any one of clauses 1 to 10 wherein Y is CR₂ₐ.

Clause 13. The compound according to any one of clauses 1 to 10 or 12 wherein Z is N.

Clause 14. The compound according to any one of clauses 1 to 12 wherein Z is CR₃ₐ.

Clause 15. The compound according to any one of clauses 1 to 8 wherein X is N, Y is CR₂ₐ and Z is N.

Clause 16. The compound according to any one of clauses 1 to 8 wherein X is N, Y is CR₂ₐ and Z is CR₃ₐ.

Clause 17. The compound according to any one of clauses 1 to 8 wherein X is CH, Y is N and Z is CR₃ₐ.

Clause 18. The compound according to any one of clauses 1 to 8 wherein X is CH, Y is CR₂ₐ and Z is CR₃ₐ.

Clause 19. The compound according to any one of clauses 1 to 8 wherein X is CH, Y is CR₂ₐ and Z is N.

Clause 20. The compound according to any one of clauses 1 to 19 wherein R₁ₐ is R₁ₐₐ.

Clause 21. The compound according to clause 20 wherein R₁ₐₐ is NR₃₂ₐR₃₃ₐ, R₃₂ₐ is C₁₋₃alkyl and R₃₃ₐ is C₁₋₃alkyl.

Clause 22. The compound according to clause 21 wherein R₃₂ₐ is methyl or ethyl, such as methyl.

Clause 23. The compound according to either clause 21 or 22 wherein R₃₃ₐ is methyl or ethyl, such as methyl.

Clause 24. The compound according to clause 21 wherein R₃₂ₐ is methyl and R₃₃ₐ is methyl.

Clause 25. The compound according to clause 20 wherein R₁ₐₐ is NR₃₂ₐR₃₃ₐ and wherein R₃₂ₐ and R₃₃ₐ together with the nitrogen atom to which they are attached form a C₃₋₅heterocycloalkyl.

Clause 26. The compound according to clause 25 wherein the C₃₋₅heterocycloalkyl is selected from the group consisting of azirdine, azetidine and pyrrolidine.

Clause 27. The compound according to any one of clauses 1 to 19 wherein R₁ₐ is R_{1ba}.

Clause 28. The compound according to any one of clauses 1 to 19 wherein R₁ₐ is C₁₋₅alkyl.

Clause 29. The compound according to any one of clauses 1 to 19 wherein R₁ₐ is C₀₋₂alkyleneC₃₋₅cycloalkyl which cycloalkyl is optionally substituted by CH₃.

Clause 30. The compound according to clause 29 wherein R₁ₐ is C₀₋₂alkyleneC₃₋₅cycloalkyl.

Clause 31. The compound according to clause 29 wherein R₁ₐ is C₀₋₂alkyleneC₃₋₅cycloalkyl which cycloalkyl is substituted by CH₃.

Clause 32. The compound according to any one of clauses 29 to 31 wherein R₁ₐ is C₃₋₅cycloalkyl optionally substituted by CH₃.

Clause 33. The compound according to any one of clauses 29 to 31 wherein R₁ₐ is C₁alkyleneC₃₋₅cycloalkyl optionally substituted by CH₃.

Clause 34. The compound according to any one of clauses 29 to 31 wherein R₁ₐ is C₂alkyleneC₃₋₅cycloalkyl optionally substituted by CH₃.

Clause 35. The compound according to any one of clauses 1 to 19 wherein R₁ₐ is cyclopropyl, cyclopropyl substituted by CH₃ at the point of attachment, cyclobutyl, methyl or ethyl.

Clause 36. The compound according to clause 35 wherein R₁ₐ is cyclopropyl, methyl or ethyl.

Clause 37. The compound according to clause 36 wherein R₁ₐ is cyclopropyl.

Clause 38. The compound according to any one of clauses 1 to 19 wherein R₁ₐ is CF₃.

Clause 39. The compound according to any one of clauses 1 to 38 wherein R₂ₐ is H.

Clause 40. The compound according to any one of clauses 1 to 38 wherein R₂ₐ is halo, such as F, Cl or Br e.g. Cl or Br.

Clause 41. The compound according to any one of clauses 1 to 38 wherein R₂ₐ is C₁₋₂alkyl such as CH₃.

Clause 42. The compound according to any one of clauses 1 to 38 wherein R₂ₐ is OC₁₋₂alkyl such as OCH₃.

Clause 43. The compound according to any one of clauses 1 to 38 wherein R₂ₐ is C₁₋₂ haloalkyl such as CF₃.

Clause 44. The compound according to any one of clauses 1 to 38 wherein R₂ₐ is OC₁₋₂ haloalkyl such as OCF₃.

Clause 45. The compound according to any one of clauses 1 to 44 wherein R₃ₐ is H.

Clause 46. The compound according to any one of clauses 1 to 44 wherein R₃ₐ is halo.

Clause 47. The compound according to clause 46 wherein R₃ₐ is fluoro.

Clause 48. The compound according to any one of clauses 1 to 44 wherein R₃ₐ is CH₃.

Clause 49. The compound according to any one of clauses 1 to 44 wherein R₃ₐ is OCH₃.

Clause 50. The compound according to any one of clauses 1 to 44 wherein R₃ₐ is CF₃.

Clause 51. The compound according to any one of clauses 1 to 44 wherein R₃ₐ is OCF₃.

Clause 52. The compound according to any one of clauses 1 to 51 wherein at least one of R₂ₐ and R₅ₐ is H.

Clause 53. The compound according to any one of clauses 1 to 52 wherein R₄ₐ and R₅ₐ are R₄ₐₐ and R₅ₐₐ.

Clause 54. The compound according to clause 53 wherein R₄ₐₐ and R₅ₐₐ together with the carbon atom to which they are attached form a C₃₋₆cycloalkyl which is substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR₂₁ₐR₂₂ₐ.

Clause 55. The compound according to clause 54 wherein R₄ₐₐ and R₅ₐₐ together with the carbon atom to which they are attached form a C₃₋₆cycloalkyl which is substituted by one substituent.

Clause 56. The compound according to either clause 54 or 55 wherein each substituent is independently selected from the group consisting of C₁₋₃alkyl, oxo, OH, C₁₋₃alkylOH, C₁₋₃ haloalkyl, halo, OC₁₋₃haloalkyl, OC₁₋₃alkyl and NR₂₁ₐR₂₂ₐ.

Clause 57. The compound according to clause 56 wherein each substituent is independently selected from the group consisting of oxo, OH, halo, OC₁₋₃alkyl and NR₂₁ₐR₂₂ₐ.

Clause 58. The compound according to clause 57 wherein each substituent is independently selected from the group consisting of oxo, OH, fluoro and NR₂₁ₐR₂₂ₐ.

Clause 59. The compound according to clause 53 wherein R₄ₐₐ and R₅ₐₐ together with the carbon atom to which they are attached form a C₃₋₆cycloalkyl wherein one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl.

Clause 60. The compound according to clause 53 wherein R₄ₐₐ and R₅ₐₐ together with the carbon atom to which they are attached form a C₃₋₆heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl.

Clause 61. The compound according to either clause 59 or 60 wherein R₄ₐₐ and R₅ₐₐ together with the carbon atom to which they are attached form a C₃₋₆cycloalkyl or C₃₋₆heterocycloalkyl which is substituted by one substituent.

Clause 62. The compound according to either clause 59 or 60 wherein R₄ₐₐ and R₅ₐₐ together with the carbon atom to which they are attached form a C₃₋₆cycloalkyl or C₃₋₆ heterocycloalkyl which is unsubstituted.

Clause 63. The compound according to any one of clauses 59, 60 or 61 wherein each substituent is independently selected from the group consisting of C₁₋₂alkyl or OCH₃.

Clause 64. The compound according to any one of clauses 59 to 63 wherein a spirocyclic ring system is formed by the C₃₋₆cycloalkyl or C₃₋₆heterocycloalkyl ring and a further C₃₋₆cycloalkyl ring.

Clause 65. The compound according to any one of clauses 59 to 63 wherein a spirocyclic ring system is formed by the C₃₋₆cycloalkyl or C₃₋₆heterocycloalkyl ring and a further C₃₋₆heterocycloalkyl ring.

Clause 66. The compound according to any one of clauses 59 or 61 to 65 wherein the C₃₋₆cycloalkyl formed by R₄ₐₐ and R₅ₐₐ together with the carbon atom to which they are attached is cyclopropyl.

Clause 67. The compound according to any one of clauses 59 or 61 to 65 wherein the C₃₋₆cycloalkyl formed by R₄ₐₐ and R₅ₐₐ together with the carbon atom to which they are attached is cyclobutyl.

Clause 68. The compound according to any one of clauses 59 or 61 to 65 wherein the C₃₋₆cycloalkyl formed by R₄ₐₐ and R₅ₐₐ together with the carbon atom to which they are attached is cyclopentyl.

Clause 69. The compound according to any one of clauses 59 or 61 to 65 wherein the C₃₋₆cycloalkyl formed by R₄ₐₐ and R₅ₐₐ together with the carbon atom to which they are attached is cyclohexyl.

Clause 70. The compound according to any one of clauses 60 to 65 wherein the C₃₋₆heterocycloalkyl formed by R₄ₐₐ and R₅ₐₐ together with the carbon atom to which they are attached is heterocyclopropyl.

Clause 71. The compound according to any one of clauses 60 to 65 wherein the C₃₋₆heterocycloalkyl formed by R₄ₐₐ and R₅ₐₐ together with the carbon atom to which they are attached is heterocyclobutyl.

Clause 72. The compound according to any one of clauses 60 to 65 wherein the C₃₋₆heterocycloalkyl formed by R₄ₐₐ and R₅ₐₐ together with the carbon atom to which they are attached is heterocyclopentyl.

Clause 73. The compound according to any one of clauses 60 to 65 wherein the C₃₋₆heterocycloalkyl formed by R₄ₐₐ and R₅ₐₐ together with the carbon atom to which they are attached is heterocyclohexyl.

Clause 74. The compound according to any one of clauses 59 or 61 to 69 wherein one of the carbons is quaternary and is attached to a 5-membered dioxalane ring to form the following structure:

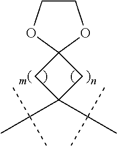

wherein m is 1 or 2 and n is 0, 1 or 2.

Clause 75. The compound according to any one of clauses 54 to 58 wherein R₂₁ₐ is H.

Clause 76. The compound according to any one of clauses 54 to 58 wherein R₂₁ₐ is C₁₋₅alkyl, such as methyl, ethyl or propyl.

Clause 77. The compound according to any one of clauses 54 to 58 wherein R₂₁ₐ is C(O)C₁₋₅alkyl, such as C(O)CH₃.

Clause 78. The compound according to any one of clauses 54 to 58 wherein R₂₁ₐ is C(O)OC₁₋₅alkyl, such as C(O)OCH₃ or C(O)Otert-butyl.

Clause 79. The compound according to any one of clauses 54 to 58 wherein R₂₁ₐ is C₁₋₃ alkylOC₁₋₂alkyl such as CH₂CH₂OCH₃.

Clause 80. The compound according to any one of clauses 54 to 58 wherein R₂₁ₐ is C₁₋₄ haloalkyl such as CH₂CHF₂.

Clause 81. The compound according to any one of clauses 54 to 58 wherein R₂₁ₐ is C₄₋₆heterocycloalkyl such as oxetane, tetrahydrofuran or tetrahydropyran e.g. oxetane.

Clause 82. The compound according to any one of clauses 54 to 58 or 75 to 81 wherein R₂₂ₐ is H.

Clause 83. The compound according to any one of clauses 54 to 58 or 75 to 81 wherein R₂₂ₐ is CH₃.

Clause 84. The compound according to clause 53 wherein R₄ₐₐ and R₅ₐₐ together with the carbon atom to which they are attached form a C₃₋₆heterocycloalkyl comprising one nitrogen atom, wherein said nitrogen atom is substituted by —S(O)₂R₂₉ₐ.

Clause 85. The compound according to clause 84 wherein the C₃₋₆heterocycloalkyl is piperidinyl and the nitrogen atom is in the 4-position relative to the quaternary carbon:

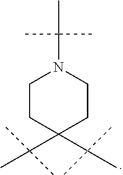

Clause 86. The compound according to either clause 84 or 85 wherein R₂₉ₐ is C₁₋₃alkyl.

Clause 87. The compound according to clause 86 wherein R₂₉ₐ is methyl.

Clause 88. The compound according to either clause 84 or 85 wherein R₂₉ₐ is N(C₁₋₃alkyl)₂ e.g. N(CH₃)₂.

Clause 89. The compound according to either clause 84 or 85 wherein R₂₉ₐ is 5 or 6 membered heteroaryl wherein the 5 or 6 membered heteroaryl is optionally substituted by methyl such as pyrazolyl substituted by methyl.

Clause 90. The compound according to any one of clauses 1 to 52 wherein R₄ₐ and R₅ₐ are R_{4ba} and R_{5ba}.

Clause 91. The compound according to any one of clauses 1 to 52 or 90 wherein R_{4ba} is H.

Clause 92. The compound according to any one of clauses 1 to 52 or 90 wherein R_{4ba} is C₁₋₆alkyl.

Clause 93. The compound according to clause 92 wherein R_{4ba} is methyl or ethyl.

Clause 94. The compound according to any one of clauses 1 to 52 or 90 wherein R_{4ba} is C₁₋₆alkylOH.

Clause 95. The compound according to any one of clauses 1 to 52 or 90 wherein R_{4ba} is C₁₋₆haloalkyl such as CF₃.

Clause 96. The compound according to any one of clauses 1 to 52 or 90 wherein R_{4ba} is C₀₋₂alkyleneC₃₋₆cycloalkyl.

Clause 97. The compound according to any one of clauses 1 to 52 or 90 wherein R_{4ba} is C₀₋₂alkyleneC₃₋₆heterocycloalkyl.

Clause 98. The compound according to any one of clauses 1 to 52 or 90 wherein R_{4ba} is C₁₋₃ alkyleneOC₁₋₃alkyl.

Clause 99. The compound according to clause 98 wherein R_{4ba} is C₂alkyleneOC₁₋₃alkyl.

Clause 100. The compound according to clause 99 wherein R_{4ba} is CH₂CH₂OCH₃.

Clause 101. The compound according to any one of clauses 1 to 52 or 90 wherein R_{4ba} is halo.

Clause 102. The compound according to clause 101 wherein R_{4ba} is fluoro.

Clause 103. The compound according to any one of clauses 1 to 52 or 90 wherein R_{4ba} is OC₁₋₆haloalkyl, such as OC₁₋₄haloalkyl.

Clause 104. The compound according to any one of clauses 1 to 52 or 90 wherein R_{4ba} is OC₀₋₂alkyleneC₃₋₆cycloalkyl.

Clause 105. The compound according to any one of clauses 1 to 52 or 90 wherein R_{4ba} is OC₀₋₂alkyleneC₃₋₆heterocycloalkyl.

Clause 108. The compound according to any one of clauses 1 to 52 or 90 wherein R_{4ba} is OC₁₋₆alkyl, in particular OC₁₋₄alkyl.

Clause 107. The compound according to any one of clauses 1 to 52 or 90 wherein R_{4ba} is NR₂₁ₐR₂₂ₐ.

Clause 108. The compound according to clause 107 wherein R₂₁ₐ is H, CH₃, C(O)CH₃, C(O)OCH₃ or C(O)Otert-butyl.

Clause 109. The compound according to clause 107 wherein R₂₁ₐ is C₁₋₃alkylOC₁₋₂alkyl such as CH₂CH₂OCH₃.

Clause 110. The compound according to clause 107 wherein R₂₁ₐ is C₁₋₄haloalkyl such as CH₂CHF₂.

Clause 111. The compound according to clause 107 wherein R₂₁ₐ is C₄₋₈heterocycloalkyl such as oxetane, tetrahydrofuran or tetrahydropyran e.g. oxetane.

Clause 112. The compound according to any one of clauses 107 or 111 wherein R₂₂ₐ is H or CH₃ such as H.

Clause 113. The compound according to any one of clauses 107 to 112 wherein R₂₁ₐ is C(O)OCH₃ and R₂₂ₐ is H, R₂₁ₐ is C(O)CH₃ and R₂₂ₐ is H, R₂₁ₐ and R₂₂ₐ are both CH₃, or R₂₁ₐ and R₂₂ₐ are both H.

Clause 114. The compound according to any one of clauses 1 to 52 or 90 wherein R_{4ba} is H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R_{4ba} and R_{5ba} together with the carbon atom to which they are attached form a C₃₋₆cycloalkyl or C₃₋₆heterocycloalkyl.

Clause 115. The compound according to any one of clauses 1 to 52 or 90 wherein R_{4ba} is halo, OC₁₋₆haloalkyl, OC₀₋₂alkyleneC₃₋₆cycloalkyl, OC₀₋₂alkyleneC₃₋₆heterocycloalkyl, OC₁₋₆alkyl or NR₂₁ₐR₂₂ₐ.

Clause 116. The compound according to any one of clauses 1 to 52 or 90 to 115 wherein R_{5ba} is H.

Clause 117. The compound according to any one of clauses 1 to 52 or 90 to 115 wherein R_{5ba} is C₁₋₆alkyl.

Clause 118. The compound according to clause 117 wherein R_{5ba} is methyl or ethyl.

Clause 119. The compound according to any one of clauses 1 to 52 or 90 to 115 wherein R_{5ba} is C₁₋₆alkylOH.

Clause 120. The compound according to any one of clauses 1 to 52 or 90 to 115 wherein R_{5ba} is C₁₋₆haloalkyl such as CF₃.

Clause 121. The compound according to any one of clauses 1 to 52 or 90 to 115 wherein R_{5ba} is C₀₋₂alkyleneC₃₋₆cycloalkyl.

Clause 122. The compound according to any one of clauses 1 to 52 or 90 to 115 wherein R_{5ba} is C₀₋₂alkyleneC₃₋₆heterocycloalkyl.

Clause 123. The compound according to any one of clauses 1 to 52 or 90 to 115 wherein R_{5ba} is C₁₋₃alkyleneOC₁₋₃alkyl, such as C₂alkyleneOC₁₋₃alkyl e.g. CH₂CH₂OCH₃.

Clause 124. The compound according to any one of clauses 1 to 52 or 90 to 115 wherein R_{5ba} is halo.

Clause 125. The compound according to clause 124 wherein R_{5ba} is fluoro.

Clause 126. The compound according to any one of clauses 1 to 52 or 90 to 115 wherein R_{5ba} is OC₁₋₆haloalkyl, such as OC₁₋₄haloalkyl.

Clause 127. The compound according to any one of clauses 1 to 52 or 90 to 115 wherein R_{5ba} is OC₀₋₂alkyleneC₃₋₆cycloalkyl.

Clause 128. The compound according to any one of clauses 1 to 52 or 90 to 115 wherein R_{5ba} is OC₀₋₂alkyleneC₃₋₆heterocycloalkyl.

Clause 129. The compound according to any one of clauses 1 to 52 or 90 to 115 wherein R_{5ba} is OC₁₋₆alkyl, in particular OC₁₋₄alkyl.

Clause 130. The compound according to any one of clauses 1 to 52 or 90 to 115 wherein R_{5ba} is NR₂₁ₐR₂₂ₐ.

Clause 131. The compound according to clause 130 wherein R₂₁ₐ is H, CH₃, C(O)CH₃, C(O)OCH, or C(O)Otert-butyl.

Clause 132. The compound according to clause 130 wherein R₂₁ₐ is C₁₋₃alkylOC₁₋₂alkyl such as CH₂CH₂OCH₃.

Clause 133. The compound according to clause 130 wherein R₂₁ₐ is C₁₋₄haloalkyl such as CH₂CHF₂.

Clause 134. The compound according to clause 130 wherein R₂₁, is C₄₋₆heterocycloalkyl such as oxetane, tetrahydrofuran or tetrahydropyran e.g. oxetane.

Clause 135. The compound according to any one of clauses 130 to 134 wherein R₂₂ₐ is H or CH₃ such as H.

Clause 136. The compound according to any one of clauses 130 to 134 wherein R₂₁ₐ is C(O)OCH₃ and R₂₂ₐ is H, R₂₁ₐ is C(O)CH₃ and R₂₂ₐ is H, R₂₁ₐ and R₂₂ₐ are both CH₃, or R₂₁ₐ and R₂₂ₐ are both H.

Clause 137. The compound according to any one of clauses 1 to 52 or 90 to 115 wherein R_{5ba} is H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R₄ and R₅ together with the carbon atom to which they are attached form a C₃₋₆cycloalkyl or C₃₋₆heterocycloalkyl.

Clause 138. The compound according to any one of clauses 1 to 52 or 90 to 115 wherein R_{5ba} is halo, OC₁₋₆haloalkyl, OC₀₋₂alkyleneC₃₋₆cycloalkyl, OC₀₋₂alkyleneC₃₋₆heterocycloalkyl, OC₁₋₆alkyl or NR₂₁ₐR₂₂ₐ.

Clause 139. The compound according to any one of clauses 1 to 52 or 90 to 116 wherein R_{4ba} and R_{5ba} are both H.

Clause 140. The compound according to any one of clauses 1 to 52, 93 or 118 wherein R_{4ba} and R_{5ba} are both methyl.

Clause 141. The compound according to any one of clauses 1 to 52, 93 or 118 wherein R_{4ba} and R_{5ba} are both ethyl.

Clause 142. The compound according to any one of clauses 1 to 52, 102 or 125 wherein R_{4ba} and R_{5ba}, are both fluoro.

Clause 143. The compound according to any one of clauses 1 to 52,102 or 116 wherein R_{4ba} is ethyl and R_{5ba} is H.

Clause 144. The compound according to any one of clauses 1 to 52, 102 or 118 wherein R_{4ba} is fluoro and R_{5ba} is ethyl.

Clause 145. The compound according to any one of clauses 1 to 52, 100 or 116 wherein R_{4ba} is CH₂CH₂OCH₃ and R_{5ba} is H.

Clause 148. The compound according to any one of clauses 143 to 145 wherein R_{4ba} and R_{5ba} are arranged in an S configuration.

Clause 147. The compound according to any one of clauses 1 to 52 or 90 wherein R_{4ba} and R_{5ba} together with the carbon atom to which they are attached form a C₃₋₆cycloalkyl.

Clause 148. The compound according to clause 147 wherein R_{4ba} and R_{5ba} together with the carbon atom to which they are attached form a cyclopropyl ring or a cyclopentyl ring, such as a cyclopentyl ring.

Clause 149. The compound according to any one of clauses 1 to 52 or 90 wherein R_{4ba} and R_{5ba} together with the carbon atom to which they are attached form a C₃₋₆heterocycloalkyl, such as heterocyclohexyl, such as tetrahydropyranal.

Clause 150. The compound according to any one of clauses 1 to 149 wherein Ar1a is phenyl.

Clause 151. The compound according to anyone of clauses 1 to 149 wherein Ar1a is 2-pyridyl.

Clause 152. The compound according to any one of clauses 1 to 149 wherein Ar1a is 3-pyridyl.

Clause 153. The compound according to any one of clauses 1 to 152 wherein Ar2a is 3-pyridyl.

Clause 154. The compound according to any one of clauses 1 to 152 wherein Ar2a is 2,5-pyrazinyl.

Clause 155. The compound according to any one of clauses 1 to 154 wherein R₁₀ₐ is H.

Clause 156. The compound according to any one of clauses 1 to 154 wherein R₁₀ₐ is halo such as fluoro or chloro.

Clause 157. The compound according to any one of clauses 1 to 154 wherein R₁₀ₐ is C₁₋₃alkyl.

Clause 158. The compound according to clause 157 wherein R₁₀ₐ is C₁₋₂alkyl such as CH₃.

Clause 159. The compound according to any one of clauses 1 to 154 wherein R₁₀ₐ is C₁₋₂ haloalkyl such as CF₃.

Clause 160. The compound according to any one of clauses 1 to 154 wherein R₁₀ₐ is OC₁₋₂alkyl such as OCH₃.

Clause 161. The compound according to any one of clauses 1 to 154 wherein R₁₀ₐ is OC₁₋₂ haloalkyl such as OCF₃.

Clause 162. The compound according to any one of clauses 1 to 154 wherein R₁₀ₐ is CN.

Clause 163. The compound according to any one of clauses 1 to 162 wherein R₁₁ₐ is H.

Clause 164. The compound according to any one of clauses 1 to 162 wherein R₁₁ₐ is F.

Clause 165. The compound according to any one of clauses 1 to 162 wherein R₁₁ₐ is Cl.

Clause 166. The compound according to any one of clauses 1 to 162 wherein R₁₁ₐ is C₁₋₂ alkyl.

Clause 167. The compound according to clause 166 wherein R₁₁ₐ is CH₃.

Clause 168. The compound according to any one of clauses 1 to 162 wherein R₁₁ₐ is CF₃.

Clause 169. The compound according to any one of clauses 1 to 162 wherein R₁₁ₐ is OCH₃.

Clause 170. The compound according to any one of clauses 1 to 162 wherein R₁₁ₐ is CN.

Clause 171. The compound according to any one of clauses 1 to 170 wherein R₁₂ₐ is H.

Clause 172. The compound according to any one of clauses 1 to 170 wherein R₁₂ₐ is halo such as fluoro or chloro.

Clause 173. The compound according to any one of clauses 1 to 170 wherein R₁₂ₐ is C₁₋₄ alkyl such as CH₃.

Clause 174. The compound according to any one of clauses 1 to 170 wherein R₁₂ₐ is C₂₋₄alkenyl.

Clause 175. The compound according to any one of clauses 1 to 170 wherein R₁₂ₐ is C₀₋₂alkyleneC₃₋₅cycloalkyl such as C₀alkyleneC₃cycloalkyl.

Clause 176. The compound according to any one of clauses 1 to 170 wherein R₁₂ₐ is OC₁₋₄ alkyl such as methoxy, ethoxy or isopropoxy.

Clause 177. The compound according to any one of clauses 1 to 170 wherein R₁₂ₐ is OC₀₋₂alkyleneC₃₋₅cycloalkyl such as OC₀alkyleneC₃cycloalkyl.

Clause 178. The compound according to any one of clauses 1 to 170 wherein R₁₂ₐ is C₁₋₄ haloalkyl such as CF₃.

Clause 179. The compound according to any one of clauses 1 to 170 wherein R₁₂ₐ is OC₁₋₄ haloalkyl such as OCH₂CF₃ or OCHF₂.

Clause 180. The compound according to any one of clauses 1 to 170 wherein R₁₂ₐ is OH.

Clause 181. The compound according to any one of clauses 1 to 170 wherein R₁₂ₐ is C₁₋₄ alkylOH.

Clause 182. The compound according to any one of clauses 1 to 170 wherein R₁₂ₐ is SO₂C₁₋₂alkyl.

Clause 183. The compound according to any one of clauses 1 to 170 wherein R₁₂ₐ is NHC(O)C₁₋₃alkyl.

Clause 184. The compound according to any one of clauses 1 to 170 wherein R₁₂ₐ is NR₂₃ₐR₂₄ₐ.

Clause 185. The compound according to clause 184 wherein R₂₄ₐ is H or C₁₋₂alkyl such as H or CH₃.

Clause 186. The compound according to clause 184 or 185 wherein R₂₄ₐ is H or C₁₋₂alkyl such as CH₃ or ethyl.

Clause 187. The compound according to any one of clauses 184 to 186 wherein R₂₃ₐ is H and R₂₄ₐ is ethyl; or R₂₃ₐ is CH₃ and R₂₄ is CH₃.

Clause 188. The compound according to any one of clauses 1 to 170 wherein R₁₂ₐ is CN.

Clause 189. The compound according to any one of clauses 1 to 170 wherein R₁₂ₐ is OCH₂CH₂N(CH₃)₂.

Clause 190. The compound according to any one of clauses 1 to 170 wherein R₁₂ₐ is a C₃₋₆heterocycloalkyl comprising one nitrogen located at the point of attachment to Ar2a.

Clause 191. The compound according to any one of clauses 1 to 170 wherein R₁₂ₐ together with a nitrogen atom to which it is attached forms an N-oxide (N⁺—O⁻).

Clause 192. The compound according to any one of clauses 1 to 170 wherein R₁₂ₐ is C(O)N(C₁₋₂alkyl)₂.

Clause 193. The compound according to any one of clauses 1 to 192 wherein R₁₃ₐ is H.

Clause 194. The compound according to any one of clauses 1 to 192 wherein R₁₃ₐ is halo such as fluoro or chloro e.g. fluoro.

Clause 195. The compound according to any one of clauses 1 to 194 when R_{1ba} is methyl, at least one of R_{4ba}, R_{5ba}, R_{10ba}, R_{11ba}, R_{12ba} and R_{13ba} is other than H.

Clause 196. The compound according to any one of clauses 1 to 195 wherein at least one, such as only one, nitrogen atom in any of the C₃₋₆heterocycloalkyl rings, such as only one of the C₃₋₆heterocycloalkyl rings is substituted, for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl, C(O)NHC₁₋₄ haloalkyl such as C(O)OtBu, C₁₋₄alkylCN such as CH₂CN, C(O)C₁₋₃alkylOC₁₋₂alkyl such as C(O)CH₂OCH₃, or C₁₋₂alkylC(O)OC₁₋₄alkyl such as CH₂C(O)OCH₂CH₃.

Clause 197. The compound according to any one of clauses 1 to 195 wherein all nitrogen atoms in all C₃₋₆heterocycloalkyl rings are not substituted.

Clause 198. The compound according to any one of clauses 1 to 197 wherein at least one, such as only one, sulphur atom in any of the C₃₋₆heterocycloalkyl rings, such as only one of the C₃₋₆heterocycloalkyl rings is substituted, for example by one oxygen atom to form S═O or by two oxygen atoms to form S(O)₂.

Clause 199. The compound according to any one of clauses 1 to 197 wherein all sulphur atoms in all C₃₋₆heterocycloalkyl rings are not substituted.

Clause 200. The compound according to any one of clauses 1 to 199 wherein R₁ₐ is R₁ₐₐ and R₄ₐ and R₅ₐ are R₄ₐₐ and R₅ₐₐ.

Clause 201. The compound according to any one of clauses 1 to 199 wherein R₁ₐ is R₁ₐₐ and Aₐ is —NH—, —CH₂NH— or —NHCH₂—.

Clause 202. The compound according to any one of clauses 1 to 199 wherein R₄ₐ and R₅ₐ are R₄ₐₐ and R₅ₐₐ and Aₐ is —NH—, —CH₂NH— or —NHCH₂—.

Clause 203. The compound according to any one of clauses 1 to 199 wherein R₁ₐ is R₁ₐₐ, R₄ₐ and R₅ₐ are R₄ₐₐ and R₅ₐₐ and Aₐ is —NH—, —CH₂NH— or —NHCH₂—.

Clause 204. A compound of the examples P226, P227, P228, P229, P230, P235, P242, P244, P248, P251, P254, P255, P256, P258, P260, P261, P288, P289, P290, P291, P292, P293, P294, P295, P296, P297, P298, P299, P300, P301, P302, P303, P304, P305, P306, P307, P308, P309, P310, P311, P312, P313, P314, P315, P316, P317 and P318.

Clause 205. A compound of formula (II-a):

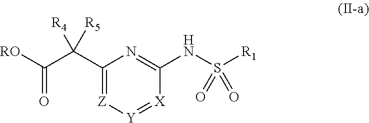

wherein R₁, X, Y, Z, R₄ and R₅ are as defined in any preceding clause and R is H, C₁₋₆alkyl (e.g. methyl and ethyl) or benzyl, or a salt, such as a pharmaceutically acceptable salt, thereof.

Clause 206. A compound of formula (XX-a):

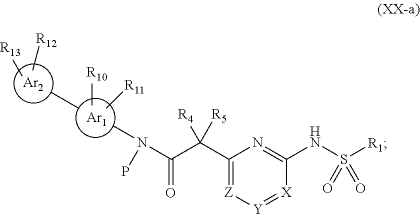

wherein Ar1, Ar2, R₁, X, Y, Z, R₄, R₅, R₁₀, R₁₁, R₁₂ and R₁₃ are as defined in any preceding clause and P is a nitrogen protecting group such as para-methoxybenzyl, or a salt, such as a pharmaceutically acceptable salt, thereof.

Clause 207. A compound of formula (XXIV-a):

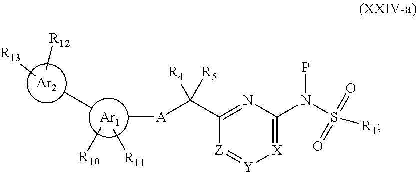

wherein Ar1, Ar2, A, R₁, X, Y, Z, R₄, R₅, R₁₀, R₁₁, R₁₂ and R₁₃ are as defined in any preceding clause and P is a nitrogen protecting group such as para-methoxybenzyl, or a salt such as a pharmaceutically acceptable salt, thereof.

Clause 208. A compound of formula (XXXI-a):

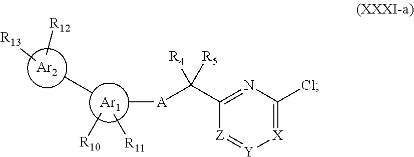

wherein Ar1, Ar2, A, X, Y, Z, R₄, R₅, R₁₀, R₁₁, R₁₂ and R₁₃ are as defined in any preceding clause, or a salt, such as a pharmaceutically acceptable salt, thereof.

Clause 209. A compound of formula (XXXXII-a):

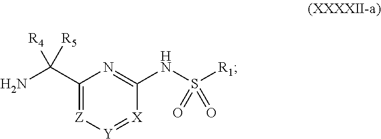

wherein R₁, X, Y, Z, R₄ and R₅ are as defined in any preceding clause, or a salt, such as a pharmaceutically acceptable salt, thereof.

Clause 210. A compound of formula (LI-a):

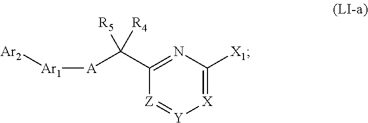

wherein Ar1, Ar2, A, R₄ and R₅ are as defined in any preceding clause and X₁ is Cl or Br, or a salt, such as a pharmaceutically acceptable salt, thereof.

Clause 211. A compound of formula (LVIII-a):

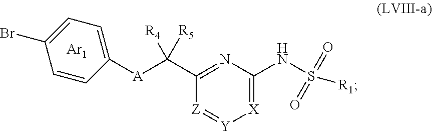

wherein R₁, Ar1, A, X, Y, Z, R₄ and R₅ are as defined in any preceding clause, or a salt, such as a pharmaceutically acceptable salt, thereof.

Clause 212. A compound of formula (XXXIII-a):

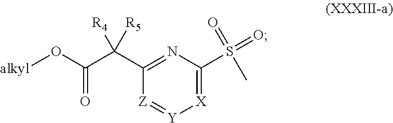

wherein X, Y, Z, R₄ and R₅ are as defined in any preceding clause and alkyl is C₁₋₄alkyl such as methyl or ethyl, e.g. methyl, or a salt, such as a pharmaceutically acceptable salt, thereof.

Clause 213. A compound of formula (LXXXIII-a):

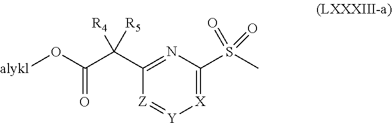

wherein X, Y, Z, R₄ and R₅ are as defined in any preceding clause and alkyl is C₁₋₄alkyl such as methyl or ethyl, e.g. methyl, or a salt, such as pharmaceutically acceptable salt, thereof.

Clause 214. A compound selected from the group consisting of:
a compound of formula (XXXIV-a):

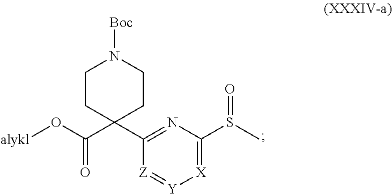

wherein and alkyl is C₁₋₄alkyl such as methyl or ethyl, e.g. methyl;
a compound of formula (LXXIII-a):

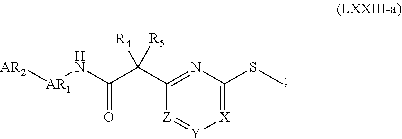

and
a compound of formula (LXXIV-a):

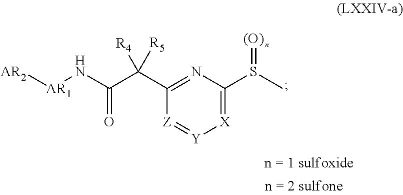

wherein X, Y, Z, R₄, R₅, Ar1 and Ar2 are as defined in any preceding clause;

and salts such as pharmaceutically acceptable salts, thereof.

Clause 215. A compound of formula (LXXI-a):

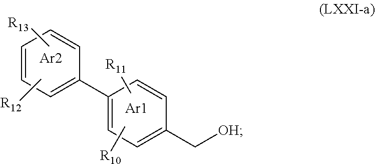

wherein Ar1, Ar2, R₁₀, R₁₁, R₁₂ and R₁₃ are as defined in any preceding clause, or a salt such as a pharmaceutically acceptable salt, thereof.

Clause 216. A compound of formula (LXXII-a):

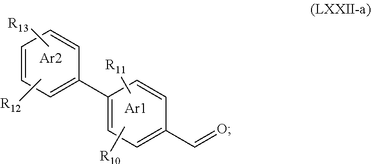

wherein Ar1, Ar2, R₁₀, R₁₁, R₁₂ and R₁₃ are as defined in any preceding clause, or a salt such as a pharmaceutically acceptable salt, thereof.

Clause 217. A compound of INTC186 to INTC218 and INTC232 to INTC247, or salt such as pharmaceutically acceptable salt thereof.

Clause 218. The compound of formula (I) according to clause 1 wherein the compound of formula (I) is a compound of formula (I-b).

Clause 219. The compound according to clause 218 wherein R_{1b} is R_{1ab}.

Clause 220. The compound according to clause 219 wherein R_{1ab} is NR_{32b}R_{33b} and wherein R_{32b} is C₁₋₃alkyl and R_{33b} is C₁₋₃alkyl.

Clause 221. The compound according to clause 220 wherein R_{32b} is methyl or ethyl.

Clause 222. The compound according to clause 221 wherein R_{32b} is methyl.

Clause 223. The compound according to any one of clauses 220 or 222 wherein R_{33b} is methyl or ethyl such as methyl.

Clause 224. The compound according to any one of clauses 220 to 223 wherein R_{32b} is methyl and R_{33b} is methyl.

Clause 225. The compound according to clause 219 wherein R_{1ab} is NR₃₂ₐR_{33b} and R_{32b} and R_{33b} together with the nitrogen atom to which they are attached form a C₃₋₅heterocycloalkyl.

Clause 226. The compound according to clause 225 wherein the C₃₋₅heterocycloalkyl is selected from the group consisting of aziridine, azetidine and pyrrolidine.

Clause 227. The compound according to clause 218 wherein R_{1b} is R_{1bb}.

Clause 228. The compound according to clause 227 wherein R_{1bb} is C₁₋₅alkyl such as CH₃ or ethyl.

Clause 229. The compound according to clause 228 wherein R_{1bb} is CH₃.

Clause 230. The compound according to any one of clauses 227 wherein R_{1bb} is C₀₋₂alkyleneC₃₋₅cycloalkyl which cycloalkyl is optionally substituted by CH₃.

Clause 231. The compound according to clause 230, wherein R_{1bb} is C₀₋₂alkyleneC₃₋₅cycloalkyl.

Clause 232. The compound according to clause 230, wherein R_{1bb} is C₀₋₂alkyleneC₃₋₅cycloalkyl which cycloalkyl is substituted by CH₃.

Clause 233. The compound according to any one of clauses 230 to 232, wherein R_{1bb} is C₃₋₅cycloalkyl, optionally substituted by CH₃.

Clause 234. The compound according to any one of clauses 230 to 232, wherein R_{1bb} is C₁alkyleneC₃₋₅cycloalkyl which cycloalkyl is optionally substituted by CH₃.

Clause 235. The compound according to any one of clauses 230 to 232, wherein R_{1bb} is C₂alkyleneC₃₋₅cycloalkyl which cycloalkyl is optionally substituted by CH₃.

Clause 236. The compound according to any one of clauses 230 to 235, wherein R_{1bb} is C₀₋₂alkyleneC₃cycloalkyl which cycloalkyl is optionally substituted by CH₃.

Clause 237. The compound according to any one of clauses 230 to 235 wherein R_{1bb} is C₀₋₂alkyleneC₄cycloalkyl which cycloalkyl is optionally substituted by CH₃.

Clause 238. The compound according to any one of clauses 230 to 235 wherein R_{1bb} is C₀₋₂alkyleneC₃cycloalkyl which cycloalkyl is optionally substituted by CH₃.

Clause 239. The compound according to any one of clauses 230 to 238 wherein R_{1bb} is cyclopropyl, cyclopropyl substituted by CH₃ at the point of attachment, cyclopropylmethylene, cyclobutyl, cyclopentyl, CH₃, ethyl, isopropyl, sec-butyl or tert-butyl, especially cyclopropyl, cyclopropyl substituted by CH₃ at the point of attachment, cyclobutyl, CH₃, ethyl or isopropyl.

Clause 240. The compound according to any one of clauses 239 wherein R_{1bb} is cyclopropyl, cyclopropyl substituted by CH₃ at the point of attachment, cyclobutyl, CH₃, isopropyl, sec-butyl or tert-butyl.

Clause 241. The compound according to any clause 240 wherein R_{1bb} is cyclopropyl, cyclopropyl substituted by CH₃ at the point of attachment, cyclobutyl or isopropyl.

Clause 242. The compound according to any one of clauses 218 to 241 wherein R_{3b} is H.

Clause 243. The compound according to any one of clauses 218 to 241 wherein R_{3b} is chloro or fluoro.

Clause 244. The compound according to any one of clauses 218 to 241 wherein R_{3b} is CH₃.

Clause 245. The compound according to any one of clauses 218 to 241 wherein R_{3b} is OCH₃.

Clause 246. The compound according to any one of clauses 218 to 241 wherein R_{3b} is CF₃.

Clause 247. The compound according to any one of clauses 218 to 241 wherein R_{3b} together with R_{5bb} forms a 5- or 6-membered cycloalkyl.

Clause 248. The compound according to clause 247 wherein R_{b} together with Ru forms a 5-membered cycloalkyl.

Clause 249. The compound according to any one of clauses 218 to 248 wherein R_{4b} and R_{5b} are R_{4ab} and R_{5ab}.

Clause 250. The compound according to clause 249 wherein R_{4ab} and R_{5ab} together with the carbon atom to which they are attached form a C₃₋₆cycloalkyl which is substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21b}R_{22b}.

Clause 251. The compound according to clause 250 wherein R_{4ab} and R_{5ab} together with the carbon atom to which they are attached form a C₃₋₆cycloalkyl which is substituted by one substituent.

Clause 252. The compound according to clause 250 or 251 wherein each substituent is independently selected from the group consisting of C₁₋₃alkyl, oxo, OH, C₁₋₃alkylOH, C₁₋₃ haloalkyl, halo, OC₁₋₃haloalkyl, OC₁₋₃alkyl and NR_{21b}R_{22b}.

Clause 253. The compound according to clause 252 wherein each substituent is independently selected from the group consisting of oxo, OH, halo, OC₁₋₃alkyl and NR_{21b}R_{22b}, such as oxo, OH, fluoro and NR_{21b}R_{22b}.

Clause 254. The compound according to any one of clauses 250 to 253 wherein R_{21b} is H.

Clause 255. The compound according to any one of clauses 250 to 253 wherein R_{21b} is C₁₋₅alkyl, such as methyl, ethyl or propyl.

Clause 256. The compound according to any one of clauses 250 to 253 wherein R_{21b} is C(O)C₁₋₅alkyl, such as C(O)CH₃.

Clause 257. The compound according to any one of clauses 250 to 253 wherein R_{21b} is C(O)OC₁₋₅alkyl, such as C(O)OCH₃ or C(O)Otert-butyl.

Clause 258. The compound according to any one of clauses 250 to 253 wherein R_{21b} is C₁₋₃ alkylOC₁₋₂alkyl such as CH₂CH₂OCH₃.

Clause 259. The compound according to any one of clauses 250 to 253 wherein R_{21b} is C₁₋₄ haloalkyl such as CH₂CHF₂.

Clause 260. The compound according to any one of clauses 250 to 253 wherein R_{21b} is C₄₋₆heterocycloalkyl such as oxetane, tetrahydrofuran or tetrahydropyran e.g. oxetane.

Clause 261. The compound according to any one of clauses 250 to 260 wherein R_{22b} is H.

Clause 262. The compound according to any one of clauses 250 to 260 wherein R_{22b} is CH₃.

Clause 263. The compound according to clause 249 wherein R_{4ab} and R_{5ab} together with the carbon atom to which they are attached form a C₃₋₆cycloalkyl wherein one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl.

Clause 264. The compound according to clause 249 wherein R_{4ab} and R_{5ab} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heteroycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl.

Clause 265. The compound according to either clause 263 or 264 wherein R_{4ab} and R_{5ab}together with the carbon atom to which they are attached form a C₃₋₆cycloalkyl or C₃₋₆heteroycloalkyl which is substituted by one substituent.

Clause 266. The compound according to clause 263 or 264 wherein R_{4ab} and R_{5ab} together with the carbon atom to which they are attached form a C₃₋₆cycloalkyl or C₃₋₆heteroycloalkyl which is unsubstituted.

Clause 267. The compound according to any one of clauses 263 to 266 wherein each substituent is independently selected from the group consisting of C₁₋₂alkyl or OCH₃.

Clause 268. The compound according to any one of clauses 263 to 267 wherein a spirocyclic ring system is formed by the C₃₋₆cycloalkyl or C₃₋₆heteroycloalkyl ring and a further C₃₋₆cycloalkyl ring.

Clause 269. The compound according to any one of clauses 263 to 267 wherein a spirocyclic ring system is formed by the C₃₋₆cycloalkyl or C₃₋₆heteroycloalkyl ring and a further C₃₋₆heterocycloalkyl ring.

Clause 270. The compound according to any one of clauses 263 or 265 to 269 wherein the C₃₋₆cycloalkyl formed by R_{4ab} and R_{5ab} together with the carbon atom to which they are attached is cyclopropyl.

Clause 271. The compound according to any one of clauses 263 or 265 to 269 wherein the C₃₋₆cycloalkyl formed by R_{4ab} and R_{5ab} together with the carbon atom to which they are attached is cyclobutyl.

Clause 272. The compound according to any one of clauses 263 or 265 to 269 wherein the C₃₋₆cycloalkyl formed by R_{4ab} and R_{5ab} together with the carbon atom to which they are attached is cyclopentyl.

Clause 273. The compound according to any one of clauses 263 or 265 to 269 wherein the C₃₋₆cycloalkyl formed by R_{4ab} and R_{5ab} together with the carbon atom to which they are attached is cyclohexyl.

Clause 274. The compound according to any one of clauses 264 to 269 wherein the C₃₋₆heterocycloalkyl formed by R_{4ab} and R_{5ab} together with the carbon atom to which they are attached is heterocyclopropyl.

Clause 275. The compound according to any one of clauses 264 to 269 wherein the C₃₋₆heterocycloalkyl formed by R_{4ab} and R_{5ab} together with the carbon atom to which they are attached is heterocyclobutyl.

Clause 276. The compound according to any one of clauses 264 to 269 wherein the C₃₋₆heterocycloalkyl formed by R_{4ab} and R_{5ab} together with the carbon atom to which they are attached is heterocyclopentyl.

Clause 277. The compound according to any one of clauses 264 to 269 wherein the C₃₋₆heterocycloalkyl formed by R_{4ab} and R_{5ab} together with the carbon atom to which they are attached is heterocyclohexyl.

Clause 278. The compound according to any one of clauses 263 or 265 to 273 wherein one of the carbons is quaternary and is attached to a 5-membered dioxalane ring to form the following structure:

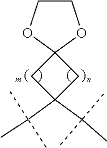

wherein m is 1 or 2 and n is 0, 1 or 2.

Clause 279. The compound according to clause 249 wherein R_{4ab} and R_{5ab} together with the carbon atom to which they are attached form a C₃₋₆heterocycloalkyl comprising one nitrogen atom, wherein said nitrogen atom is substituted by —S(O)₂R_{29b}.

Clause 280. The compound according to clause 279 wherein the C₃₋₆heterocycloalkyl is piperidinyl and the nitrogen atom is in the 4-position relative to the quaternary carbon:

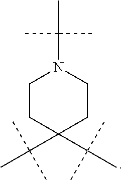

Clause 281. The compound according to clause 279 or 280wherein R_{29b} is C₁₋₃alkyl.

Clause 282. The compound according to clause 281 wherein R_{29b} is methyl.

Clause 283. The compound according to either clause 279 or 280wherein R is N(C₁₋₃alkyl)₂ e.g. N(CH₃)₂.

Clause 284. The compound according to either clause 279 or 280 wherein R_{29b} is 5 or 6 membered heteroaryl wherein the 5 or 6 membered heteroaryl is optionally substituted by methyl such as pyrazolyl substituted by methyl.

Clause 285. The compound according to any one of clauses 218 to 248 wherein R_{4b} and R_{5b} are R_{4bb} and R_{5bb}.

Clause 286. The compound according to any one of clauses 218 to 248 and 285 wherein R_{4bb} is H and R_{3b} together with R_{5bb} form a 5- or 6-membered oxygen-containing heterocycloalkyl such as tetrahydrofuranyl or tetrahydropyranyl.

Clause 287. The compound according to any one of clauses 218 to 246 and 285 wherein R_{4bb} together with R_{5bb} form a C₃₋₆cycloalkyl.

Clause 288. The compound according to clause 287 wherein R_{4bb} together with R_{5bb} form cyclopropyl or cyclopentyl.

Clause 289. The compound according to any one of clauses 218 to 246 and 285 wherein R_{4bb} together with R_{5bb} form a C₃₋₆heterocycloalkyl.

Clause 290. The compound according to clause 289 wherein R_{4bb} together with R_{5bb} form heterocyclopentyl or heterocyclohexyl, such as tetrahydrofuranyl, pyrrolidinyl, tetrahydropyranyl or piperidinyl such as tetrahydropyranyl or piperidinyl.

Clause 291. The compound according to any one of clauses 218 to 248 and 285, wherein R_{4bb} is C₁₋₆alkyl, in particular C₁₋₄alkyl such as methyl, ethyl or propyl (n-propyl or isopropyl).

Clause 292. The compound according to any one of clauses 218 to 248 and 285, wherein R_{4bb} is C₀₋₂alkyleneC₃₋₆cycloalkyl, such as C₃₋₆cycloalkyl, C₁alkyleneC₃₋₆cycloalkyl or C₂alkyleneC₃₋₆cycloalkyl.

Clause 293. The compound according to any one of clauses 218 to 248 and 285, wherein R_{4bb} is OC₁₋₆alkyl, in particular OC₁₋₄alkyl, such as methoxy or isopropoxy.

Clause 294. The compound according to any one of clauses 218 to 248 and 285, wherein R_{4bb} is OC₀₋₂alkyleneC₃₋₆cycloalkyl such as OC₃₋₆cycloalkyl, OC₁alkyleneC₃₋₆cycloalkyl or OC₂alkyleneC₃₋₆cycloalkyl.

Clause 295. The compound according to any one of clauses 218 to 248 and 285, wherein R_{4bb} is C₁₋₃alkyleneOC₁₋₃alkyl, in particular C₁₋₂alkyleneOC₁₋₂alkyl, such as CH₂CH₂OCH₃.

Clause 298. The compound according to any one of clauses 218 to 248 and 285, wherein R_{4bb} is C₁₋₆haloalkyl, in particular C₁₋₄haloalkyl.

Clause 297. The compound according to any one of clauses 218 to 248 and 285, wherein R_{4bb} is OC₁₋₆haloalkyl, in particular OC₁₋₄haloalkyl.

Clause 298. The compound according to clause 218 to 248 and 285 wherein R_{4bb} is H.

Clause 299. The compound according to clause 218 to 248 and 285 wherein R_{4bb} is halo such as F.

Clause 300. The compound according to clause 218 to 248 and 285 wherein R_{4bb} is C₁₋₆alkylOH, such as CH₂CH₂OH.

Clause 301. The compound according to clause 218 to 248 and 285 wherein R_{4b} is NR_{21b}R_{22b}.

Clause 302. The compound according to clause 301 wherein R_{21b} is H.

Clause 303. The compound according to clause 301 wherein R_{21b} is C₁₋₅alkyl, such as methyl, ethyl or propyl.

Clause 304. The compound according to clause 301 wherein R_{21b} is C(O)C₁₋₅alkyl, such as C(O)CH₃.

Clause 305. The compound according to clause 301 wherein R_{21b} is C(O)OC₁₋₅alkyl, such as C(O)OCH₃ or C(O)Otert-butyl.

Clause 308. The compound according to clause 301 wherein R_{21b} is C₁₋₃alkylOC₁₋₂alkyl such as CH₂CH₂OCH₃.

Clause 307. The compound according to clause 301 wherein R_{21b} is C₁₋₄haloalkyl such as CH₂CHF₂.

Clause 308. The compound according to clause 301 wherein R_{21b} is C₄₋₆heterocycloalkyl such as oxetane, tetrahydrofuran or tetrahydropyran e.g. oxetane.

Clause 309. The compound according to clause 301 to 308 wherein R_{22b} is H.

Clause 310. The compound according to clause 301 to 308 wherein R_{22b} is CH₃.

Clause 311. The compound according to any one of clauses 218 to 248 and 285 wherein R_{4bb} is C₀₋₂alkyleneC₃₋₆heterocycloalkyl.

Clause 312. The compound according to any one of clauses 218 to 311 wherein any nitrogen atom in the C₃₋₆heterocycloalkyl ring is substituted, for example by C₁₋₄alkyl, C(O)H, C(O)C₁₋₄ alkyl, C(O)OC₁₋₄alkyl, C(O)OC₁₋₄alkylaryl such as C(O)OBz, C(O)NHC₁₋₄alkyl, C(O)NHC₁₋₄ alkylaryl such as C(O)NHBz, an Fmoc group, C(O)C₁₋₄haloalkyl, C(O)OC₁₋₄haloalkyl or C(O)NHC₁₋₄haloalkyl such as C(O)OtBu, C₁₋₄alkylCN such as CH₂CN, C(O)C₁₋₃alkylOC₁₋₂alkyl such as C(O)CH₂OCH₃, and C₁₋₂alkylC(O)OC₁₋₄alkyl such as CH₂C(O)OCH₂CH₃.

Clause 313. The compound according to any one of clauses 218 to 311 wherein any nitrogen atom in the C₃₋₆heterocycloalkyl ring is not substituted.

Clause 314. The compound according to any one of clauses 218 to 313 wherein any sulphur atom in the C₃₋₆heterocycloalkyl ring is substituted, for example by one oxygen atom to form S═O or by two oxygen atoms to form S(O)₂.

Clause 315. The compound according to any one of clauses 218 to 313 wherein any sulphur atom in the C₃₋₆heterocycloalkyl ring is not substituted.

Clause 316. The compound according to any one of clauses 218 to 246 and 285 to 315, wherein R_{5bb} is H, methyl, ethyl or fluoro e.g. H, methyl or ethyl.

Clause 317. The compound according to clause 316, wherein R_{5bb} is H.

Clause 318. The compound according to any one of clauses 218 to 246 and 285 to 316 wherein R_{4bb} and R_{5bb} are methyl.

Clause 319. The compound according to any one of clauses 218 to 246 and 285 to 316 wherein R_{4bb} and R_{5bb} are ethyl.

Clause 320. The compound according to any one of clauses 218 to 246 and 285 wherein R_{4bb} is H and R_{5bb} and R_{6b} are a C₂₋₃alkylene chain forming a 5- or 6-membered ring, such as a 5-membered ring.

Clause 321. The compound according to any one of clauses 218 to 246 and 285 wherein R_{4bb} is O and R_{5bb} is absent.

Clause 322. The compound according to any one of clauses 218 to 246 and 285 wherein R_{4bb} is ethyl and R_{5bb} is H and the groups are arranged in the S configuration.

Clause 323. The compound according to any one of clauses 218 to 322 wherein A_{b} is A_{ab}.

Clause 324. The compound according to clause 323 wherein A_{ab} is —NR_{6b}CH₂—.

Clause 325. The compound according to clause 323 wherein A_{ab} is —NR_{6b}—.

Clause 326. The compound according to any one of clauses 218 to 322 wherein A_{b} is A_{bb}.

Clause 327. The compound according to clause 324 or 325 wherein R_{6b} is H.

Clause 328. The compound according to clause 324 or 325 wherein R_{6b} is C₁₋₃alkyl such as CH₃.

Clause 329. The compound according to clause 324 or 325 wherein R_{6b} together with R_{11b} in the ortho-position to group A_{b} are a C₂alkylene chain forming a 5-membered ring.

Clause 330. The compound according to any one of clauses 218 to 329 wherein Ar1b is phenyl.

Clause 331. The compound according to any one of clauses 218 to 329 wherein Ar1b is pyridyl.

Clause 332. The compound according to clause 331 wherein Ar1b is 2-pyridyl.

Clause 333. The compound according to any one of clauses 218 to 329 wherein Ar1b is pyridazinyl.

Clause 334. The compound according to any one of clauses 218 to 329 wherein Ar1b is pyrimidinyl.

Clause 335. The compound according to any one of clauses 218 to 329 wherein Ar1b is pyrazinyl.

Clause 336. The compound according to any one of clauses 218 to 335 wherein R_{10b} is H.

Clause 337. The compound according to any one of clauses 218 to 335 wherein R_{10b} is chloro or fluoro, such as fluoro.

Clause 338. The compound according to any one of clauses 218 to 335 wherein R_{10b} is methoxy or ethoxy such as methoxy.

Clause 339. The compound according to any one of clauses 218 to 335 wherein R_{10b} is C₁₋₃alkyl.

Clause 340. The compound according to clause 339 wherein R_{10b} is methyl.

Clause 341. The compound according to any one of clauses 218 to 335 wherein R_{10b} is OCF₃.

Clause 342. The compound according to any one of clauses 218 to 335 wherein R_{10b} is CF₃.

Clause 343. The compound according to any one of clauses 218 to 335 wherein R_{10b} is CN.

Clause 344. The compound according to any one of clauses 337 to 343 wherein R_{10b} is in the ortho or meta position with respect to group A_{b}, such as the ortho position with respect to group A_{b}.

Clause 345. The compound according to any one of clauses 218 to 334 wherein R_{11b} is H.

Clause 346. The compound according to any one of clauses 218 to 334 wherein R_{11b} is fluoro.

Clause 347. The compound according to any one of clauses 218 to 334 wherein R_{11b} is methyl.

Clause 348. The compound according to clause 348 or 347 wherein R_{11b} is in the ortho or meta position with respect to group A_{b}, such as the ortho position with respect to group A_{b}.

Clause 349. The compound according to any one of clauses 218 to 348 wherein Ar2b is phenyl.

Clause 350. The compound according to any one of clauses 218 to 348 wherein Ar2b is pyridyl.

Clause 351. The compound according to clause 350 wherein Ar2b is 3-pyridyl.

Clause 352. The compound according to any one of clauses 218 to 348 wherein Ar2b is pyridazinyl.

Clause 353. The compound according to any one of clauses 218 to 348 wherein Ar2b is pyrimidinyl.

Clause 354. The compound according to any one of clauses 218 to 348 wherein Ar2b is pyrazinyl.

Clause 355. The compound according to any one of clauses 218 to 354 wherein R₁₂ₐ is H.

Clause 356. The compound according to any one of clauses 218 to 354 wherein R₁₂ₐ is halo, such as F or Cl.

Clause 357. The compound according to any one of clauses 218 to 354 wherein R₁₂ₐ is C₁₋₄ alkyl, such as ethyl or methyl, especially methyl.

Clause 358. The compound according to any one of clauses 218 to 354 wherein R₁₂ₐ is C₂₋₄alkynyl, such as C≡CH.

Clause 359. The compound according to any one of clauses 218 to 354 wherein R_{12b} is C₀₋₂alkyleneC₃₋₅cycloalkyl, such as cyclopropyl.

Clause 360. The compound according to any one of clauses 218 to 354 wherein R₁₂ₐ is OC₁₋₄alkyl such as methoxy, ethoxy, isopropoxy or n-propoxy.

Clause 361. The compound according to any one of clauses 218 to 354 wherein R_{12b} is OC₀₋₂alkyleneC₃₋₅cycloalkyl, such as cyclopropoxyl or cyclobutoxy.

Clause 362. The compound according to any one of clauses 218 to 354 wherein R₁₂ₐ is OCH₂CH₂N(CH₃)₂

Clause 363. The compound according to any one of clauses 218 to 354 wherein R_{12b} is C₁₋₄ alkylOH, such as CH₂OH or C(CH₃)₂OH.

Clause 364. The compound according to any one of clauses 218 to 354 wherein R_{12b} is CN.

Clause 365. The compound according to any one of clauses 218 to 354 wherein R_{12b} is C₁₋₃ alkyleneOC₁₋₃alkyl.

Clause 366. The compound according to any one of clauses 218 to 354 wherein R_{12b} is C₁₋₄ haloalkyl, such as CF₃.

Clause 387. The compound according to any one of clauses 218 to 354 wherein R_{12b} is OC₁₋₄haloalkyl, such as OCF₃, OCHF₂ or OCH₂CF₃.

Clause 368. The compound according to any one of clauses 218 to 354 wherein R_{12b} is C(═O)C₁₋₂alkyl, such as C(═O)CH₃.

Clause 369. The compound according to any one of clauses 218 to 354 wherein R_{12b} is NR_{23b}R_{24b}.

Clause 370. The compound according to clause 369 wherein R_{12b} is N(CH₃)₂.

Clause 371. The compound according to clause 369 wherein R_{12b} is N(H)Et.

Clause 372. The compound according to any one of clauses 218 to 354 wherein R_{12b} is SO₂C₁₋₄alkyl.

Clause 373. The compound according to clause 372 wherein R_{12b} is SO₂CH₃.

Clause 374. The compound according to any one of clauses 218 to 354 wherein R_{12b} is C(O)N(CH₃)₂.

Clause 375. The compound according to any one of clauses 218 to 354 wherein R_{12b} is NHC(O)C₁₋₃alkyl such as NHC(O)CH₃.

Clause 376. The compound according to any one of clauses 218 to 354 wherein R_{12b} is OH.

Clause 377. The compound according to any one of clauses 218 to 354 wherein R_{12b} is C₃₋₆heterocycloalkyl comprising one nitrogen located at the point of attachment to Ar2b such as a C₅heterocycloalkyl or C₆heterocycloalkyl, and in particular pyrrolidinyl.

Clause 378. The compound according to any one of clauses 218 to 354 wherein R_{12b} together with a nitrogen atom to which it is attached forms an N-oxide (N⁺—O⁻).

Clause 379. The compound according to any one of clauses 218 to 378 wherein R_{12b} is in the meta position of Ar2b.

Clause 380. The compound according to any one of clauses 218 to 378 wherein R_{12b} is in the ortho position of Ar2b.

Clause 381. The compound according to any one of clauses 218 to 380 wherein R_{13b} is H.

Clause 382. The compound according to any one of clauses 218 to 380 wherein R_{13b} is methyl.

Clause 383. The compound according to clause 382 wherein R_{13b} is in the ortho position with respect to Ar1b.

Clause 384. The compound according to clause 382 wherein R_{13b} is in the para position with respect to Ar1b.

Clause 385. The compound according to any one of clauses 218 to 384 wherein R_{1b} is R_{1ab} and R_{4b} and R_{5b} are R_{4ab} and R_{5ab}.

Clause 386. The compound according to any one of clauses 218 to 384 wherein R_{1b} is R_{1ab} and A_{b} is A_{ab}.

Clause 387. The compound according to any one of clauses 218 to 384 wherein R_{1b} and R_{1ab}are R_{4ab} and R_{5ab} and A_{b} is A_{ab}.

Clause 388. The compound according to any one of clauses 218 to 384 wherein R_{1b} is R_{1ab}, R_{4b} and R_{5b} are R_{4ab} and R_{5ab} and A_{b} is A_{ab}.

Clause 389. A compound of the example T486.

Clause 390. A compound of formula (II-b):

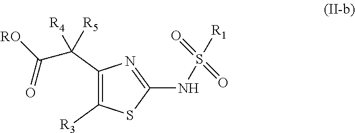

wherein R₁, R₃, R₄ and R₅ are as defined in any one of clauses 218 to 389 and R is H, C₁₋₆alkyl (e.g. methyl and ethyl) or benzyl.

Clause 391. A compound of formula (X-b):

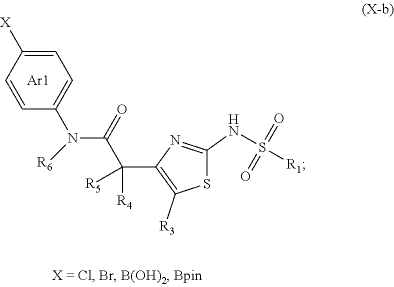

wherein Ar1, R₁, R₃, R₄, R₅ and Rₐ are as defined in any one of clauses 218 to 389.

Clause 392. A compound of formula (XII-b):

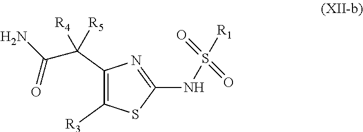

wherein R₁, R₃, R₄ and R₅ are as defined in any one of clauses 218 to 389.

Clause 393. The compound of formula (I) according to clause 1 wherein the compound of formula (I) is a compound of formula (I-c).

Clause 394. The compound according to clause 393 wherein R_{1c} is R_{1ac}.

Clause 395. The compound according to clause 394 wherein R_{1ac} is NR_{32c}R_{33c} and wherein R_{32c} is C₁₋₃alkyl and R_{33c} is C₁₋₃alkyl.

Clause 396. The compound according to clause 395 wherein R_{32c} is methyl or ethyl.

Clause 397. The compound according to clause 396 wherein R_{32c} is methyl.

Clause 398. The compound according to any one of clauses 395 to 397 wherein R_{32c} is methyl or ethyl.

Clause 399. The compound according to clause 398 wherein R_{32c} is methyl.

Clause 400. The compound according to any one of clauses 397 to 399 wherein R_{32c} is methyl and R_{33c} is methyl.

Clause 401. The compound according to clause 394 wherein R_{1ac} is NR_{32c}R_{33c} and R_{32c} and R_{33c} together with the nitrogen atom to which they are attached form a C₃₋₅heterocycloalkyl.

Clause 402. The compound according to clause 401 wherein the C₃₋₅heterocycloalkyl is selected from the group consisting of aziridine, azetidine and pyrrolidine.

Clause 403. The compound according to clause 393 wherein R_{1c} is R_{1bc}.

Clause 404. The compound according to clause 403 wherein R_{1bc} is C₁₋₅alkyl.

Clause 405. The compound according to clause 403 wherein R_{1bc} is C₁₋₃alkyleneOC₁₋₂alkyl.

Clause 406. The compound according to clause 405 wherein R_{1bc} is C₁₋₂alkyleneOC₁₋₂alkyl.

Clause 407. The compound according to clause 403 wherein R_{1bc} is C₀₋₂alkyleneC₃₋₅cycloalkyl which cycloalkyl is optionally substituted by CH₃.

Clause 408. The compound according to clause 407 wherein R_{1bc} is C₀₋₁alkyleneC₃₋₄cycloalkyl which cycloalkyl is optionally substituted by CH₃.

Clause 409. The compound according to clause 408 wherein R_{1bc} is C₀₋₁alkyleneC₃₋₄cycloalkyl.

Clause 410. The compound according to any one of clauses 407 or 409 wherein R_{1bc} is C₃₋₄ cycloalkyl.

Clause 411. The compound according to clause 410 wherein R_{1bc} is cyclopropyl.

Clause 412. The compound according to clause 408 wherein R_{1bc} is C₀₋₁alkyleneC₃₋₄cycloalkyl which cycloalkyl is substituted by CH₃.

Clause 413. The compound according to any one of clauses 393 to 412 wherein R_{3c} is H.

Clause 414. The compound according to any one of clauses 393 to 412 wherein R_{3c} is Me.

Clause 415. The compound according to any one of clauses 393 to 412 wherein R_{3c} is halo.

Clause 416. The compound according to clause 415 wherein R_{3c} is F.

Clause 417. The compound according to clause 415 wherein R_{3c} is Cl.

Clause 418. The compound according to any one of clauses 393 to 412 wherein R_{3c} is OC₁₋₂ alkyl.

Clause 419. The compound according to any one of clauses 393 to 412 wherein R_{3c} is OCF₃.

Clause 420. The compound according to any one of clauses 393 to 412 wherein R_{3c} is CF₃.

Clause 421. The compound according to any one of clauses 393 to 420 wherein R_{4c} and R_{5c} are R_{4ac} and R_{5ac}.

Clause 422. The compound according to clause 421 wherein R_{4ac} and R_{5ac} together with the carbon atom to which they are attached form a C₃₋₆cycloalkyl which is substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21c}R_{22c}.

Clause 423. The compound according to clause 422 wherein R_{4ac} and R_{5ac} together with the carbon atom to which they are attached form a C₃₋₆cycloalkyl which is substituted by one substituent.

Clause 424. The compound according to clause 422 or 423 wherein each substituent is independently selected from the group consisting of C₁₋₃alkyl, oxo, OH, C₁₋₃alkylOH, C₁₋₃haloalkyl, halo, OC₁₋₃haloalkyl, OC₁₋₃alkyl and NR_{21c}R_{22c}.

Clause 425. The compound according to clause 424 wherein each substituent is independently selected from the group consisting of oxo, OH, halo, OC₁₋₃alkyl and NR_{21c}R_{22c}, such as oxo, OH, fluoro and NR_{21c}R_{22c}.

Clause 426. The compound according to any one of clauses 422 to 425 wherein R_{21c} is H.

Clause 427. The compound according to any one of clauses 422 to 425 wherein R_{21c} is C₁₋₅alkyl, such as methyl, ethyl or propyl.

Clause 428. The compound according to any one of clauses 422 to 425 wherein R_{21c} is C(O)C₁₋₅alkyl, such as C(O)CH₃.

Clause 429. The compound according to any one of clauses 422 to 425 wherein R_{21c} is C(O)OC₁₋₅alkyl, such as C(O)OCH₃ or C(O)Otert-butyl.

Clause 430. The compound according to any one of clauses 422 to 425 wherein R_{21c} is C₁₋₃alkylOC₁₋₂alkyl such as CH₂CH₂OCH₃.

Clause 431. The compound according to any one of clauses 422 to 425 wherein R_{21c} is C₁₋₄ haloalkyl such as CH₂CHF₂.

Clause 432. The compound according to any one of clauses 422 to 425 wherein R_{21c} is C₄₋₆heterocycloalkyl such as oxetane, tetrahydrofuran or tetrahydropyran e.g. oxetane.

Clause 433. The compound according to any one of clauses 422 to 432 wherein R_{22c} is H.

Clause 434. The compound according to any one of clauses 422 to 432 wherein R_{22c} is CH₃.

Clause 435. The compound according to clause 421 wherein R_{4ac} and R_{5ac} together with the carbon atom to which they are attached form a C₃₋₆cycloalkyl wherein one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl.

Clause 436. The compound according to clause 421 wherein R_{4ac} and R_{5ac} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heteroycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl.

Clause 437. The compound according to either clause 435 or 436 wherein R_{4ac} and R_{5ac} together with the carbon atom to which they are attached form a C₃₋₆cycloalkyl or C₃₋₆heteroycloalkyl which is substituted by one substituent.

Clause 438. The compound according to clause 435 or 436 wherein R_{4ac} and R_{5ac} together with the carbon atom to which they are attached form a C₃₋₆cycloalkyl or C₃₋₆heteroycloalkyl which is unsubstituted.

Clause 439. The compound according to any one of clauses 435 to 438 wherein each substituent is independently selected from the group consisting of C₁₋₂alkyl or OCH₃.

Clause 440. The compound according to any one of clauses 435 to 439 wherein a spirocyclic ring system is formed by the C₃₋₆cycloalkyl or C₃₋₆heteroycloalkyl ring and a further C₃₋₆cycloalkyl ring.

Clause 441. The compound according to any one of clauses 435 to 439 wherein a spirocyclic ring system is formed by the C₃₋₆cycloalkyl or C₃₋₆heteroycloalkyl ring and a further C₃₋₆heterocycloalkyl ring.

Clause 442. The compound according to any one of clauses 435 or 437 to 441 wherein the C₃₋₆cycloalkyl formed by R_{4ac} and R_{5ac} together with the carbon atom to which they are attached is cyclopropyl.

Clause 443. The compound according to any one of clauses 435 or 437 to 441 wherein the C₃₋₆cycloalkyl formed by R_{4ac} and R_{5ac} together with the carbon atom to which they are attached is cyclobutyl.

Clause 444. The compound according to any one of clauses 435 or 437 to 441 wherein the C₃₋₆cycloalkyl formed by R_{4ac} and R_{5ac} together with the carbon atom to which they are attached is cyclopentyl.

Clause 445. The compound according to any one of clauses 435 or 437 to 441 wherein the C₃₋₆cycloalkyl formed by R_{4ac} and R_{5ac} together with the carbon atom to which they are attached is cyclohexyl.

Clause 446. The compound according to any one of clauses 436 to 441 wherein the C₃₋₆heterocycloalkyl formed by R_{4ac} and R_{5ac} together with the carbon atom to which they are attached is heterocyclopropyl.

Clause 447. The compound according to any one of clauses 436 to 441 wherein the C₃₋₆heterocycloalkyl formed by R_{4ac} and R_{5ac} together with the carbon atom to which they are attached is heterocyclobutyl.

Clause 448. The compound according to any one of clauses 436 to 441 wherein the C₃₋₆heterocycloalkyl formed by R_{4ac} and R_{5ac} together with the carbon atom to which they are attached is heterocyclopentyl.

Clause 449. The compound according to any one of clauses 436 to 441 wherein the C₃₋₆heterocycloalkyl formed by R_{4ac} and R_{5ac} together with the carbon atom to which they are attached is heterocyclohexyl.

Clause 450. The compound according to any one of clauses 435 or 437 to 445 wherein one of the carbons is quaternary and is attached to a 5-membered dioxolane ring to form the following structure:

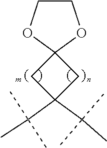

wherein m is 1 or 2 and n is 0, 1 or 2.

Clause 451. The compound according to clause 421 wherein R_{4ac} and R_{5ac} together with the carbon atom to which they are attached form a C₃₋₆heterocycloalkyl comprising one nitrogen atom, wherein said nitrogen atom is substituted by —S(O)₂R_{29c}.

Clause 452. The compound according to clause 451 wherein the C₃₋₆heterocycloalkyl is piperidinyl and the nitrogen atom is in the 4-position relative to the quaternary carbon:

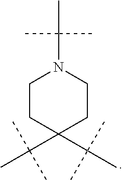

Clause 453. The compound according to clause 451 or 452 wherein R_{29c} is C₁₋₃alkyl.

Clause 454. The compound according to clause 453 wherein R_{29c} is methyl.

Clause 455. The compound according to either clause 451 or 452 wherein R₂ is N(C₁₋₃ alkyl)₂ e.g. N(CH₃)₂.

Clause 456. The compound according to either clause 451 or 452 wherein R_{29c} is 5 or 6 membered heteroaryl wherein the 5 or 6 membered heteroaryl is optionally substituted by methyl such as pyrazolyl substituted by methyl.

Clause 457. The compound according to any one of clauses 393 to 420 wherein R_{4c} and R_{5c} are R_{4bc} and R_{5bc}.

Clause 458. The compound according to any one of clauses 393 to 420 and 457 wherein R_{4bc} is H.

Clause 459. The compound according to any one of clauses 393 to 420 and 457 wherein R_{4bc} is C₁₋₄alkyl.

Clause 460. The compound according to clause 459 wherein R_{4bc} is C₁₋₄alkyl.

Clause 461. The compound according to clause 460 wherein R_{4bc} is methyl or ethyl.

Clause 462. The compound according to any one of clauses 393 to 420 and 457 wherein R_{4bc} is C₀₋₂alkyleneC₃₋₆cycloalkyl.

Clause 463. The compound according to clause 482 wherein R_{4bc} is C₀₋₂alkyleneC₃₋₅cycloalkyl.

Clause 464. The compound according to any one of clauses 393 to 420 and 457 wherein R_{4bc} is C₁₋₆alkyleneOC₁₋₃alkyl such as CH₂CH₂OCH₃.

Clause 465. The compound according to any one of clauses 393 to 420 and 457 wherein R_{4bc} is C₀₋₂alkyleneC₃₋₆heterocycloalkyl.

Clause 466. The compound according to any one of clauses 393 to 420 and 457 wherein R_{4bc} is C₁₋₆alkylOH.

Clause 467. The compound according to clause 466 wherein R_{4bc} is C₁₋₄alkylOH.

Clause 468. The compound according to clause 393 to 420 and 457 wherein R_{4bc} is C₁₋₆haloalkyl.

Clause 469. The compound according to clause 468 wherein R_{4bc} is C₁₋₄haloalkyl.

Clause 470. The compound according to any one of clauses 393 to 420 and 457 to 469 wherein R_{5bc} is H.

Clause 471. The compound according to any one of clauses 393 to 420 and 457 to 469 wherein R_{5bc} is C₁₋₆alkyl.

Clause 472. The compound according to clause 471 wherein R_{5bc} is C₁₋₄alkyl.

Clause 473. The compound according to clause 472 wherein R_{5bc} is methyl or ethyl.

Clause 474. The compound according to any one of clauses 393 to 420 and 457 to 469 wherein R_{5bc} is C₀₋₂alkyleneC₃₋₆cycloalkyl.

Clause 475. The compound according to clause 474 wherein R_{5bc} is C₀₋₂alkyleneC₃₋₅cycloalkyl.

Clause 476. The compound according to any one of clauses 393 to 420 and 457 to 469 wherein R_{5bc} is C₀₋₂akyleneC₃₋₆heterocycloalkyl.

Clause 477. The compound according to any one of clauses 393 to 420 and 457 to 469 wherein R_{5bc} is C₁₋₃alkyleneOC₁₋₃alkyl such as CH₂CH₂OCH₃.

Clause 478. The compound according to any one of clauses 393 to 420 and 457 to 469 wherein R_{5bc} is C₁₋₆alkylOH.

Clause 479. The compound according to clause 478 wherein R_{5bc} is C₁₋₄alkylOH.

Clause 480. The compound according to any one of clauses 393 to 420 and 457 to 469 wherein R_{5bc} is C₁₋₆haloalkyl.

Clause 481. The compound according to clause 480 wherein R_{5bc} is C₁₋₄haloalkyl.

Clause 482. The compound according to any one of clauses 393 to 420 wherein R_{4bc} and R_{5bc} together with the carbon atom to which they are attached form a C₃₋₆cycloalkyl such as cyclopropyl.

Clause 483. The compound according to any one of clauses 393 to 420 wherein R_{4bc} and R_{5bc} together with the carbon atom to which they are attached form a C₃₋₆heterocycloalkyl such as tetrahydropyranyl or piperidinyl.

Clause 484. The compound according to any one of clauses 393 to 483 wherein at least one, such as one, nitrogen atom of a C₃₋₆heterocycloalkyl ring is substituted, for example by C₁₋₄alkyl, C(O)H, C(O)C₁₋₄alkyl, C(O)OC₁₋₄alkyl, C(O)OC₁₋₄alkylaryl such as C(O)OBz, C(O)NHC₁₋₄alkyl, C(O)NHC₁₋₄alkylaryl such as C(O)NHBz, an Fmoc group, C(O)C₁₋₄haloalkyl, C(O)OC₁₋₄haloalkyl or C(O)NHC₁₋₄haloalkyl such as C(O)OtBu, C₁₋₄alkylCN such as CH₂CN, C(O)C₁₋₃alkylOC₁₋₂alkyl such as C(O)CH₂OCH₃, and C₁₋₂alkylC(O)OC₁₋₄alkyl such as CH₂C(O)OCH₂CH₃.

Clause 485. The compound according to any one of clauses 393 to 483 wherein any nitrogen atom in the C₃₋₆heterocycloalkyl ring is not substituted.

Clause 486. The compound according to any one of clauses 393 to 485 wherein at least one, such as one, sulphur atom of a C₃₋₆heterocycloalkyl ring is substituted, for example by one oxygen atom to form S═O or by two oxygen atoms to form S(O)₂.

Clause 487. The compound according to any one of clauses 393 to 485 wherein any sulphur atom in the C₃₋₆heterocycloalkyl ring is not substituted.

Clause 488. The compound according to any one of clauses 393 to 470 wherein R_{4bc} and R_{5bc} are both H.

Clause 489. The compound according to any one of clauses 393 to 473 wherein R_{4bc} and R_{5bc} are both methyl.

Clause 490. The compound according to any one of clauses 393 to 473 wherein R_{4bc} and R_{5bc} are both ethyl.

Clause 491. The compound according to any one of clauses 393 to 470 wherein R_{4bc} is ethyl and R_{5bc} is H.

Clause 492. The compound according to clause 491 wherein R_{4bc} and R_{5bc} are arranged in an S configuration.

Clause 493. The compound according to any one of clauses 393 to 492 wherein A_{c} is —CH₂NR_{6c}—.

Clause 494. The compound according to any one of clauses 393 to 492 wherein A_{c} is A_{bc}.

Clause 495. The compound according to clause 493 wherein R_{6c} is H.

Clause 496. The compound according to clause 493 wherein R_{6c} is C₁₋₃alkyl.

Clause 497. The compound according to clause 496 wherein R_{6c} is methyl.

Clause 498. The compound according to clause 496 wherein R_{6c} is ethyl.

Clause 499. The compound according to any one of clauses 393 to 498 wherein Ar1c is phenyl.

Clause 500. The compound according to any one of clauses 393 to 498 wherein Ar1c is 2-pyridyl.

Clause 501. The compound according to any one of clauses 393 to 500 wherein Ar2c is 3-pyridyl.

Clause 502. The compound according to any one of clauses 393 to 500 wherein Ar2c is 2,5-pyrazinyl.

Clause 503. The compound according to any one of clauses 393 to 502 wherein R_{10c} is H.

Clause 504. The compound according to any one of clauses 393 to 502 wherein R_{10c} is halo such as F.

Clause 505. The compound according to any one of clauses 393 to 502 wherein R₁₀ is C₁₋₃ alkyl such as methyl.

Clause 506. The compound according to any one of clauses 393 to 502 wherein R_{10c} is OC₁₋₂ alkyl such as OCH₃.

Clause 507. The compound according to any one of clauses 393 to 502 wherein R_{10c} is C₁₋₂ haloalkyl such as CF₃.

Clause 508. The compound according to any one of clauses 393 to 502 wherein R_{10c} is OC₁₋₂ haloalkyl.

Clause 509. The compound according to any one of clauses 393 to 502 wherein R_{10c} is CN.

Clause 510. The compound according to any one of clauses 504 to 509 wherein R_{10c} is ortho to group A_{c}.

Clause 511. The compound according to any one of clauses 393 to 510 wherein R_{11c} is H.

Clause 512. The compound according to any one of clauses 393 to 510 wherein R_{11c} is F.

Clause 513. The compound according to any one of clauses 393 to 510 wherein R_{11c} is Cl.

Clause 514. The compound according to any one of clauses 393 to 510 wherein R_{11c} is CH₃.

Clause 515. The compound according to any one of clauses 393 to 510 wherein R_{11c} is ethyl.

Clause 516. The compound according to any one of clauses 393 to 510 wherein R_{11c} is OCH₃.

Clause 517. The compound according to any one of clauses 393 to 510 wherein R_{11c} is CF₃.

Clause 518. The compound according to any one of clauses 393 to 510 wherein R_{11c} is OCF₃.

Clause 519. The compound according to any one of clauses 393 to 510 wherein R_{11c} is CN.

Clause 520. The compound according to any one of clauses 393 to 519 wherein R_{12c} is H.

Clause 521. The compound according to any one of clauses 393 to 519 wherein R_{12c} is halo such as fluoro or chloro.

Clause 522. The compound according to any one of clauses 393 to 519 wherein R_{12c} is C₁₋₄ alkyl such as CH₃ or ethyl.

Clause 523. The compound according to any one of clauses 393 to 519 wherein R_{12c} is C₂₋₄alkynyl.

Clause 524. The compound according to clause 523 wherein R_{12c} is C₂alkynyl.

Clause 525. The compound according to any one of clauses 393 to 519 wherein R_{12c} is C(═O)C₁₋₂alkyl such as C(═O)CH₃.

Clause 526. The compound according to any one of clauses 393 to 519 wherein R_{12C} is C₀₋₂alkyleneC₃₋₅cycloalkyl.

Clause 527. The compound according to any one of clauses 393 to 519 wherein R_{12c} is OC₁₋₄ alkyl such as OCH₃, OEt or OiPr.

Clause 528. The compound according to any one of clauses 393 to 519 wherein R_{12c} is C₁₋₃ alkyleneOC₁₋₃alkyl.

Clause 529. The compound according to any one of clauses 393 to 519 wherein R_{12c} is C₁₋₄ haloalkyl such as CF₃.

Clause 530. The compound according to any one of clauses 393 to 519 wherein R_{12c} is OC₁₋₄ haloalkyl such as OCH₂CF₃.

Clause 531. The compound according to any one of clauses 393 to 519 wherein R_{12c} is CN.

Clause 532. The compound according to any one of clauses 393 to 519 wherein R_{12c} is OC₀₋₂alkyleneC₃₋₅cycloalkyl.

Clause 533. The compound according to any one of clauses 393 to 519 wherein R_{12c} is OCH₂CH₂N(CH₃)₂.

Clause 534. The compound according to any one of clauses 393 to 519 wherein R_{12c} is OH.

Clause 535. The compound according to any one of clauses 393 to 519 wherein R_{12c} is C₁₋₄ alkylOH.

Clause 536. The compound according to any one of clauses 393 to 519 wherein R_{12c} is NR_{23c}R_{24c}.

Clause 537. The compound according to clause 538 wherein R_{23c} is H.

Clause 538 The compound according to clause 536 wherein R_{23c} is C₁₋₂alkyl such as CH₃.

Clause 539. The compound according to any one of clauses 536 to 538 wherein R_{24c} is H.

Clause 540. The compound according to any one of clauses 536 to 538 wherein R_{24c} is C₁₋₂ alkyl such as CH₃.

Clause 541. The compound according to any one of clauses 393 to 519 wherein R_{12c} is SO₂CH₃.

Clause 542. The compound according to any one of clauses 393 to 519 wherein R_{12c} is C(O)N(CH₃)₂.

Clause 543. The compound according to any one of clauses 393 to 519 wherein R_{12c} is NHC(O)C₁₋₃alkyl.

Clause 544. The compound according to any one of clauses 393 to 519 wherein R_{12c} is a C₃₋₆heterocycloalkyl comprising one nitrogen located at the point of attachment to Ar2c.

Clause 545. The compound according to any one of clauses 393 to 519 wherein R_{12c}together with a nitrogen atom to which it is attached forms an N-oxide (N⁺—O⁻).

Clause 546. The compound according to any one of clauses 521 to 545 wherein R_{12c} is attached at the meta position of Ar2c.

Clause 547. The compound according to any one of clauses 521 to 545 wherein R_{12c} is attached at the ortho position of Ar2c.

Clause 548. The compound according to any one of clauses 393 to 547 wherein R_{1c} is R_{1ac} and R_{4c} and R_{5c} are R_{4ac} and R_{5ac}.

Clause 549. The compound according to any one of clauses 393 to 547 wherein R_{1c} is R_{1ac} and A_{c} is A_{ac}.

Clause 550. The compound according to any one of clauses 393 to 547 wherein R_{4c} and R_{5c}are R_{4ac} and R_{5ac} and A_{c} is A_{ac}.

Clause 551. The compound according to any one of clauses 393 to 547 wherein R_{1c} is R_{1ac}, R_{4c} and R_{5c} are R_{4ac} and R_{5ac} and A_{c} is A_{ac}.

Clause 552. A compound which is R94 or R95.

Clause 553. A compound of formula (II-c):

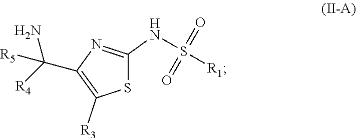

wherein R₁, R₃, R₄ and R₆ are as defined in clause 393.

Clause 554. A compound of formula (VIII-c):

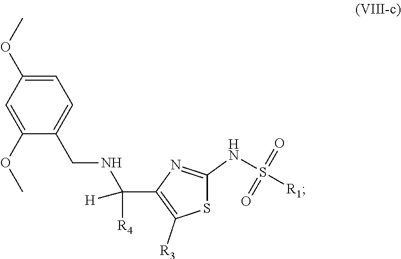

wherein R₁, R₃ and R₄ are as defined in clause 393.

Clause 555. A compound according to any one of clauses 1 to 554 which is in the form of a salt.

Clause 556. A compound according to any one of clauses 1 to 204, 218 to 389 or 393 to 552, for use as a medicament.

Clause 557. The compound according to clause 556, for use in the inhibition of CTPS1 in a subject.

Clause 558. The compound according to clause 556, for use in the reduction of T-cell and/or B-cell proliferation in a subject

Clause 559. The compound according to clause 556, for use in the treatment or prophylaxis of: inflammatory skin diseases such as psoriasis or lichen planus; acute and/or chronic GVHD such as steroid resistant acute GVHD; acute lymphoproliferative syndrome (ALPS); systemic lupus erythematosus, lupus nephritis or cutaneous lupus; or transplantation.

Clause 560. The compound according to clause 556, for use in the treatment or prophylaxis of myasthenia gravis, multiple sclerosis or scleroderma/systemic sclerosis.

Clause 561. A compound according to clause 556, for use in the treatment of cancer.

Clause 562. A method for treating cancer in a subject, by administering to a subject in need thereof a compound according to any one of clauses 1 to 204, 218 to 389 or 393 to 552.

Clause 563. Use of a compound according to any one of clauses 1 to 204, 218 to 389 or 393 to 552, in the manufacture of a medicament for the treatment of cancer in a subject.

Clause 564. The compound according to clause 561, the method according to clause 562 or the use according to clause 563 wherein the cancer is a haematological cancer.

Clause 565. The compound, method or use according to clause 564 wherein the haematological cancer is selected from the group consisting of Acute myeloid leukemia, Angioimmunoblastic T-cell lymphoma, B-cell acute lymphoblastic leukemia, Sweet Syndrome, T-cell Non-Hodgkins lymphoma (including natural killer/T-cell lymphoma, adult T-cell leukaemia/lymphoma, enteropathy type T-cell lymphoma, hepatosplenic T-cell lymphoma and cutaneous T-cell lymphoma), T-cell acute lymphoblastic leukemia, B-cell Non-Hodgkins lymphoma (including Burkitt lymphoma, diffuse large B-cell lymphoma, Follicular lymphoma, Mantle cell lymphoma, Marginal Zone lymphoma), Hairy Cell Leukemia, Hodgkin lymphoma, Lymphoblastic lymphoma, Lymphoplasmacytic lymphoma, Mucosa-associated lymphoid tissue lymphoma, Multiple myeloma, Myelodysplastic syndrome, Plasma cell myeloma, Primary mediastinal large B-cell lymphoma, chronic myeloproliferative disorders (such as chronic myeloid leukemia, primary myelofibrosis, essential thrombocytemia, polycytemia vera) and chronic lymphocytic leukemia.

Clause 566. The compound according to clause 561, the method according to clause 562 or the use according to clause 563 wherein the cancer is a non-haematological cancer such as bladder cancer, breast cancer, melanoma, neuroblastoma, malignant pleural mesothelioma and sarcoma, such as breast cancer and melanoma.

Clause 567. The compound according to clause 556, for use in enhancing recovery from vascular injury or surgery and reducing morbidity and mortality associated with neointima and restenosis in a subject.

Clause 568. A method for enhancing recovery from vascular injury or surgery and reducing morbidity and mortality associated with neointima and restenosis in a subject, by administering to a subject in need thereof a compound according to any one of clauses 1 to 204, 218 to 389 or 393 to 552.

Clause 569. Use of a compound according to any one of clauses 1 to 204, 218 to 389 or 393 to 552, in the manufacture of a medicament for enhancing recovery from vascular injury or surgery and reducing morbidity and mortality associated with neointima and restenosis in a subject.

Clause 570. A method for the inhibition of CTPS1 in a subject, which comprises administering to the subject an effective amount of a compound according to any one of clauses 1 to 204, 218 to 389 or 393 to 552.

Clause 571. Use of a compound according to any one of clauses 1 to 204, 218 to 389 or 393 to 552, in the manufacture of a medicament for the inhibition of CTPS1 in a subject.

Clause 572. A pharmaceutical composition comprising a compound according to any one of clauses 1 to 204, 218 to 389 or 393 to 552.

Clause 573. The compound, method, use or composition according to any one of clauses 556 to 572, for administration to a human subject.

Clause 574. The compound, method, use or composition according to any one of clauses 556 to 573, for administration in conjunction with a further pharmaceutically acceptable active ingredient or ingredients.

Clause 575. The compound, method, use or composition according to any one of clauses 556 to 574, for topical administration to the skin, eye or gut.

Clause 576. The compound according to any one of clauses 1 to 555, which is in natural isotopic form.

### REFERENCES

Evans, D. R. & Guy, H. I. Mammalian pyrimidine biosynthesis: fresh insights into an ancient pathway. J. Biol. Chem. 279, 33035-33038 (2004).
Fairbanks, L D. et al. Importance of ribonucleotide availability to proliferating T-lymphocytes from healthy humans. Disproportionate expansion of pyrimidine pools and contrasting effects of de novo synthesis inhibitors. J. Biol. Chem. 270, 29682-29689 (1995).
Higgins, M. J. et al. Regulation of human cytidine triphosphate synthetase 1 by glycogen synthase kinase 3. J. Biol. Chem. 282, 29493-29503 (2007).
Kursula, P. et al. Structure of the synthetase domain of human CTP synthetase, a target for anticancer therapy. Acta Crystallogr Sect F Struct Biol Cryst Commun. 62 (Pt7): 613-617 (2006).
Ueberman 1. Enzymatic amination of uridine triphosphate to cytidine triphosphate. The J. Biol. Chem. 222 (2): 765-75 (1956).
Martin E. et al. CTP synthase 1 deficiency in humans reveals its central role in lymphocytes proliferation. Nature. June 12; 510(7504):288-92 (2014). Erratum in: Nature. July 17; 511(7509):370 (2014).
McCluskey G D et al., Exploring the Potent Inhibition of CTP Synthase by Gemcitabine-5′-Triphosphate. Chembiochem. 17, 2240-2249 (2016).
Ostrander, D. B. et al. Effect of CTP synthetase regulation by CTP on phospholipid synthesis in Saccharomyces cerevisiae. J. Bio. Chem. 273, 18992-19001 (1998).
Sakamoto K et al. Identification of cytidine-5-triphosphate synthase1-selective inhibitory peptide from random peptide library displayed on T7 phage. Peptides. 2017; 94:56-63 (2017).
Salu et al. Drug-eluting stents: a new treatment in the prevention of restenosis Part I: experimental studies. Acta Cardiol, 59, 51-61 (2004).
Sousa J. E. et al. Drug-Eluting Stents. Circulation, 107 (2003) 2274 (Part I), 2283 (Part II).
Tang R. et al. CTP synthase 1, a smooth muscle-sensitive therapeutic target for effective vascular repair. Arterioscler Thromb Vasc Biol. 33(10), 1-19, (2013).
van den Berg, A. A. et al. Cytidine triphosphate (CTP) synthetase activity during cell cycle progression in normal and malignant T-lymphocytic cells. Eur. J. Cancer 31, 108-112 (1995).
van Kuilenburg, A. B. P. et al. Identification of a cDNA encoding an isoform of human CTP synthetase. Biochimica et Biophysica Acta 1492548-552 (2000).

1.-15. (canceled)

16. A compound of formula (I): 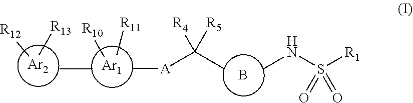
wherein ring B is selected from the group consisting of: 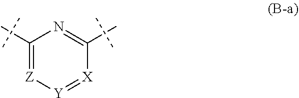
wherein X, Y and Z are as defined below; and 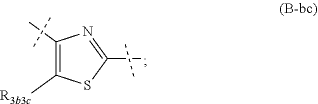
wherein R_{3b3c} is R_{3b} or R_{3c} as defined below;
wherein when B is (B-a) the compound of formula (I) is a compound of formula (I-a): 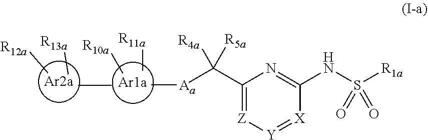
wherein:
Aₐ is Aₐₐ or A_{ba};
wherein:
Aₐₐ is an amine linker having the following structure: —NH—, —CH₂NH— or —NHCH₂—;
A_{ba} is an amide linker having the following structure: —C(═O)NH— or —NHC(═O)—;
X is N or CH;
Y is N or CR₂ₐ;
Z is N or CR₃ₐ;
with the proviso that when at least one of X or Z is N, Y cannot be N;
R₂ₐ is H, halo, C₁₋₂alkyl, OC₁₋₂alkyl, C₁₋₂haloalkyl or OC₁₋₂haloalkyl; and
R₃ₐ is H, halo, CH₃, OCH₃, CF₃ or OCF₃;
wherein at least one of R₂ₐ and R₃ₐ is H;
R₁ₐ is R₁ₐₐ or R_{1ba};
wherein:
R₁ₐₐ is NR₃₂ₐR₃₃ₐ;
R_{1ba} is C₁₋₅alkyl, C₀₋₂alkyleneC₃₋₅cycloalkyl which cycloalkyl is optionally substituted by CH₃, or CF₃;
R₄ₐ and R₅ₐ are R₄ₐₐ and R₅ₐₐ, or R_{4ba} and R_{5ba};
wherein:
R₄ₐₐ and R₅ₐₐ together with the carbon atom to which they are attached form a C₃₋₆cycloalkyl which is:
substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR₂₁ₐR₂₂ₐ; or
one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl; or
R₄ₐₐ and R₅ₐₐ together with the carbon atom to which they are attached form a C₃₋₆heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl; or
R₄ₐₐ and R₅ₐₐ together with the carbon atom to which they are attached form a C₃₋₆heterocycloalkyl comprising one nitrogen atom, wherein said nitrogen atom is substituted by —S(O)₂R₂₉ₐ; or
R_{4ba} and R_{5ba} are each independently H, C₁₋₆alkyl, C₁₋₆alkylOH, C₁₋₆haloalkyl, C₀₋₂alkyleneC₃₋₆cycloalkyl, C₀₋₂alkyleneC₃₋₆heterocycloalkyl, C₁₋₃alkyleneOC₁₋₃alkyl, or R_{4ba} and R_{5ba} together with the carbon atom to which they are attached form a C₃₋₆cycloalkyl or C₃₋₆heterocycloalkyl; and
when Aₐ is —NHC(═O)— or —NHCH₂—:
R_{4ba} and R_{5ba} may additionally be selected from halo, OC₁₋₆haloalkyl, OC₀₋₂alkyleneC₃₋₆cycloalkyl, OC₀₋₂alkyleneC₃₋₄heterocycloalkyl, OC₁₋₄alkyl and NR₂₁ₐR₂₂ₐ;
Ar1a is a 6-membered aryl or heteroaryl;
Ar2a is a 6-membered aryl or heteroaryl and is attached to Ar1a in the para position relative to group Aₐ;
R₁₀ₐ is H, halo, C₁₋₃alkyl, C₁₋₂haloalkyl, OC₁₋₂alkyl, OC₁₋₂haloalkyl or CN;
R₁₁ₐ is H, F, Cl, C₁₋₂alkyl, CF₃, OCH₃ or CN;
R₁₂ₐ is attached to Ar2 in the ortho or meta position relative to Ar1a and R₁₂ₐ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃ₐR₂₄ₐ; and
when Aₐ is —NHC(═O)—, —NH— or —NHCH₂—:
R₁₂ₐ may additionally be selected from CN, OCH₂CH₂N(CH₃)₂ and a C₃₋₆heterocycloalkyl comprising one nitrogen located at the point of attachment to Ar2a, or R₁₂ₐ together with a nitrogen atom to which it is attached forms an N-oxide (N⁺—O⁻);
R₁₃ₐ is H or halo;
R₂₁ₐ is H, C₁₋₅alkyl, C(O)C₁₋₅alkyl, C(O)OC₁₋₅alkyl, C₁₋₃alkylOC₁₋₂alkyl, C₁₋₄haloalkyl, or C₄₋₆heterocycloalkyl;
R₂₂ₐ is H or CH₃;
R₂₃ₐ is H or C₁₋₂alkyl; and
R₂₄ₐ is H or C₁₋₂alkyl
R₂₉ₐ is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl;
R₃₂ₐ is C₁₋₃alkyl and R₃₃ is C₁₋₃alkyl; or
R₃₂ₐ and R₃₃ₐ together with the nitrogen atom to which they are attached form a C₃₋₅heterocycloalkyl;
wherein
R₁ₐ is R₁ₐₐ; and/or
R₄ₐ and R₅ₐ are R₄ₐₐ and R₅ₐₐ; and/or
Aₐ is Aₐₐ; and
wherein when B is (B-bc) and R_{3b3c} is R_{3b}, the compound of formula (I) is a compound of formula (I-b): 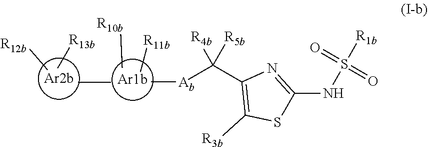
wherein:
A_{b} is A_{ab} or A_{bb};
wherein:
A_{ab} is —NR_{6b}CH₂— or —NR_{6b}—;
A_{bb} is —NR_{6b}C(═O)—;
R_{1b} is R_{1ab} or R_{1bb};
wherein:
R₁ₐ is NR_{32b}R_{33b};
R_{1bb} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R_{3b} is H, halo, CH₃, OC₁₋₂alkyl or CF₃;
or R_{3b} together with R_{5bb} forms a 5- or 6-membered cycloalkyl or 5 or 6 membered oxygen-containing heterocycloalkyl;
R_{4b} and R_{5b} are either R_{4ab} and R_{5ab} or R_{4bb} and R^{5bb};
wherein:
R_{4ab} and R_{5ab} together with the carbon atom to which they are attached form a C₃₋₄cycloalkyl which is:
substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₄heterocycloalkyl, OC₁₋₃alkyl and NR_{21b}R_{22b}; or
one of the carbons of the C₃₋₆cycloalkyl is a spiro centre such that a spirocyclic ring system is formed by the C₃₋₆cycloalkyl ring and a further C₃₋₆cycloalkyl ring or a C₃₋₄heterocycloalkyl ring, and wherein the C₃₋₆cycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl; or
R_{4ab} and R_{5ab} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heteroycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl; or
R_{4ab} and R_{5ab} together with the carbon atom to which they are attached form a C₃₋₆heterocycloalkyl comprising one nitrogen atom, wherein said nitrogen atom is substituted by —S(O)₂R_{29b}; or
R_{4ab} and R_{5ab} are each independently H, halo, C₁₋₄alkyl, C₀₋₂alkyleneC₃₋₆cycloalkyl, C₀₋₂alkyleneC₃₋₆heterocycloalkyl, OC₁₋₄alkyl, OC₀₋₂alkyleneC₃₋₆cycloalkyl, C₁₋₃alkyleneOC₁₋₃alkyl, C₁₋₆alkylOH, C₁₋₆haloalkyl, OC₁₋₆haloalkyl or NR_{21b}R_{22b},
or R_{4bb} is H and R_{5bb} together with R3b form a 5- or 6-membered cycloalkyl or 5 or 6 membered oxygen-containing heterocycloalkyl,
or R_{4bb} and R_{5bb} together with the carbon atom to which they are attached form a C₃₋₆cycloalkyl or C₃₋₆heterocycloalkyl,
or R_{4bb} is H and R_{5bb} and R_{6b} are a C₂₋₃alkylene chain forming a 5- or 6-membered ring;
or R_{4bb} is O and R_{5bb} is absent;
R_{6b} is H or C₁₋₃alkyl,
or R_{6b} together with R_{11b} when in the ortho-position to group A_{b} are a C₂alkylene chain forming a 5-membered ring,
or R_{5bb} and R_{6b} are a C₂₋₃alkylene chain forming a 5- or 6-membered ring and R_{4bb} is H;
Ar1b is 6-membered aryl or heteroaryl;
Ar2b is a 6-membered aryl or heteroaryl and is attached to Ar1b in the para position relative to group A_{b};
R_{10b} is H, halo, C₁₋₃alkyl, OC₁₋₂alkyl, C₁₋₂haloalkyl, OC₁₋₂haloalkyl or CN;
R_{11b} is H, F, Cl, CH₃, ethyl, OCH₃, CF₃, OCF₃ or CN,
or R_{11b}, when in the ortho-position to group A_{b}, together with R_{6b} are a C₂alkylene chain forming a 5-membered ring;
R_{12b} is attached to Ar2b in the ortho or meta position relative to Ar1b and R_{12b} is H, halo, C₁₋₄alkyl, C₂₋₄alkynyl, C₀₋₂alkyleneC₃₋₅cycloalkyl, OC₁₋₄alkyl, OC₀₋₂alkyleneC₃₋₅cycloalkyl, OCH₂CH₂N(CH₃)₂, OH, C₁₋₄alkylOH, CN, C₁₋₃alkyleneOC₁₋₃alkyl, C₁₋₄haloalkyl, OC₁₋₄haloalkyl, C(═O)C₁₋₂alkyl, NR_{23b}R_{24b}, SO₂C₁₋₄alkyl, SOC₁₋₄alkyl, SC₁₋₄alkyl, SH, C(O)N(CH₃)₂, NHC(O)C₁₋₃alkyl, C₃₋₆heterocycloalkyl comprising one nitrogen located at the point of attachment to Ar2b, or R_{12b} together with a nitrogen atom to which it is attached forms an N-oxide (N⁺—O⁻);
R_{13b} is H, halo, CH₃ or OCH₃;
R_{21b} is H, C₁₋₅alkyl, C(O)C₁₋₅alkyl, C(O)OC₁₋₅alkyl, C₁₋₃alkylOC₁₋₂alkyl, C₁₋₄haloalkyl, or C₄₋₆heterocycloalkyl;
R_{22b} is H or CH₃;
R_{23b} is H or C₁₋₂alkyl;
R_{24b} is H or C₁₋₂alkyl;
R_{29b} is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl; and
R_{32b} is C₁₋₃alkyl and R_{33b} is C₁₋₃alkyl; or
R_{32b} and R_{33b} together with the nitrogen atom to which they are attached form a C₃₋₅heterocycloalkyl;
wherein:
R_{1b} is R_{1ab}; and/or
R_{4b} and R_{5b} are R_{4ab} and R_{5ab}; and/or
A is A_{ab}; or
wherein when B is (B-bc) and R_{3b3c} is R_{3c}, the compound of formula (I) is a compound of formula (I-c): 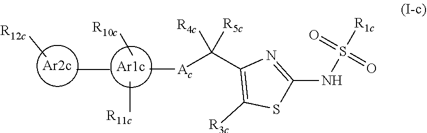
wherein:
A_{c} is A_{ac} or A_{bc};
wherein:
A_{ac} is —CH₂NR_{6c}—;
A_{bc} is —C(═O)NR_{6c}—;
R_{1c} is R_{1ac} or R_{1bc};
wherein:
R_{1ac} is NR_{32c}R_{33c};
R_{1bc} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R_{3c} is H, CH₃, halo, OC₁₋₂alkyl or CF₃;
R_{4c} and R_{5c} are either R_{4ac} and R_{5ac} or R_{4bc} and R_{5bc};
wherein:
R_{4ac} and R_{5ac} together with the carbon atom to which they are attached form a C₃₋₆cycloalkyl which is:
substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21c}R_{22c}; or
one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl; or
R_{4ac} and R_{5ac} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heteroycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl; or
R_{4ac} and R_{5ac} together with the carbon atom to which they are attached form a C₃₋₆heterocycloalkyl comprising one nitrogen atom, wherein said nitrogen atom is substituted by —S(O)₂R_{29c}; or
R_{4bc} and R_{5bc} are each independently H, C₁₋₆alkyl, C₀₋₂alkyleneC₃₋₄cycloalkyl, C₀₋₂alkyleneC₃₋₄heterocycloalkyl, C₁₋₃alkyleneOC₁₋₃alkyl, C₁₋₄alkylOH or C₁₋₆haloalkyl,
or R_{4bc} and R_{5bc} together with the carbon atom to which they are attached form a C₃₋₄cycloalkyl or C₃₋₄heterocycloalkyl ring;
R_{6c} is H or C₁₋₃alkyl;
Ar1c is a 6-membered aryl or heteroaryl;
Ar2c is a 6-membered aryl or heteroaryl and is attached to Ar1c in the para position relative to group A_{c};
R_{10c} is H, halo, C₁₋₃alkyl, OC₁₋₂alkyl, C₁₋₂haloalkyl, OC₁₋₂haloalkyl or CN;
R_{11c} is H, F, Cl, CH₃, ethyl, OCH₃, CF₃, OCF₃ or CN;
R_{12c} is attached to Ar2c in the meta or ortho position relative to Ar1c and R_{12c} is H, halo, C₁₋₄alkyl, C₂₋₄alkynyl, C(═O)C₁₋₂alkyl, C₀₋₂alkyleneC₃₋₅cycloalkyl, OC₁₋₄alkyl, C₁₋₃alkyleneOC₁₋₃alkyl, C₁₋₄haloalkyl, OC₁₋₄haloalkyl, CN, OC₀₋₂alkyleneC₃₋₅cycloalkyl, OCH₂CH₂N(CH₃)₂, OH, C₁₋₄alkylOH, NR_{23c}R_{24c}, SO₂CH₃, C(O)N(CH₃)₂, NHC(O)C₁₋₃alkyl, or a C₃₋₆heterocycloalkyl comprising one nitrogen located at the point of attachment to Ar2c, or R_{12c} together with a nitrogen atom to which it is attached forms an N-oxide (N⁺—O⁻);
R_{21c} is H, C₁₋₅alkyl, C(O)C₁₋₅alkyl, C(O)OC₁₋₅alkyl, C₁₋₃alkylOC₁₋₂alkyl, C₁₋₄haloalkyl, or C₄₋₆heterocycloalkyl;
R_{22c} is H or CH₃;
R_{23c} is H or C₁₋₂alkyl;
R_{24c} is H or C₁₋₂alkyl;
R_{29c} is C₁₋₃alkyl, C₀₋₂alkyleneC₃₋₅cycloalkyl which cycloalkyl is optionally substituted by CH₃, CF₃, N(C₁₋₃alkyl)₂, or a 5 or 6 membered heteroaryl wherein the 5 or 6 membered heteroaryl is optionally substituted by methyl; and
R_{32c} is C₁₋₃alkyl and R_{33c} is C₁₋₃alkyl; or
R_{32c} and R_{33c} together with the nitrogen atom to which they are attached form a C₃₋₅heterocycloalkyl;
wherein:
R_{1c} is R_{1ac}; and/or
R_{4c} and R_{5c} are R_{4ac} and R_{5ac}; and/or
A_{c} is A_{ac};
or a pharmaceutically acceptable salt and/or solvate thereof and/or derivative thereof.

17. The compound according to claim 16 which is a compound of formula (I): 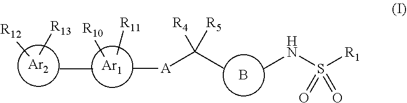
wherein ring B is selected from the group consisting of: 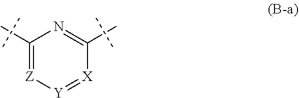
wherein X, Y and Z are as defined below; and: 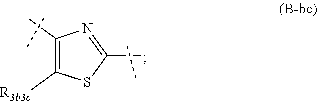
wherein R_{3b3c} is R_{3b} or R_{3c} as defined below;
wherein when B is (B-a) the compound of formula (I) is a compound of formula (I-a): 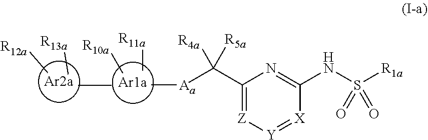
wherein:
Aₐ is Aₐₐ or A_{ba};
wherein:
Aₐₐ is an amine linker having the following structure: —NH—, —CH₂NH— or —NHCH₂—;
A_{ba} is an amide linker having the following structure: —C(═O)NH— or —NHC(═O)—;
X is N or CH;
Y is N or CR₂ₐ;
Z is N or CR₃ₐ;
with the proviso that when at least one of X or Z is N, Y cannot be N;
R₂ₐ is H, halo, C₁₋₂alkyl, OC₁₋₂alkyl, C₁₋₂haloalkyl or OC₁₋₂haloalkyl; and
R₃ₐ is H, halo, CH₃, OCH₃, CF₃ or OCF₃;
wherein at least one of R₂ₐ and R₃ₐ is H;
R₁ₐ is R₁ₐₐ or R_{1ba};
wherein:
R₁ₐₐ is NR₃₂ₐR₃₃ₐ;
R_{1ba} is C₁₋₅alkyl, C₀₋₂alkyleneC₃₋₅cycloalkyl which cycloalkyl is optionally substituted by CH₃, or CF₃;
R₄ₐ and R₅ₐ are R₄ₐₐ and R₅ₐₐ, or R_{4ba} and R_{5ba};
wherein:
R₄ₐₐ and R₅ₐₐ together with the carbon atom to which they are attached form a C₃₋₆cycloalkyl which is:
substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR₂₁ₐR₂₂ₐ; or
one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl; or
R₄ₐₐ and R₅ₐₐ together with the carbon atom to which they are attached form a C₃₋₆heterocycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heterocycloalkyl formed by R₄ₐₐ and R₅ₐₐ together with the carbon atom to which they are attached may be substituted by one or two substituents, each substituent being independently selected from the group consisting of C₁₋₃alkyl or OC₁₋₃alkyl; or
R₄ₐₐ and R₅ₐₐ together with the carbon atom to which they are attached form a C₃₋₆heterocycloalkyl comprising one nitrogen atom, wherein said nitrogen atom is substituted by —S(O)₂R₂₉ₐ; or
R_{4ba} and R_{5ba} are each independently H, C₁₋₄alkyl, C₁₋₄alkylOH, C₁₋₄haloalkyl, C₀₋₂alkyleneC₃₋₄cycloalkyl, C₀₋₂alkyleneC₃₋₄heterocycloalkyl, C₁₋₃alkyleneOC₁₋₃alkyl, or R_{4ba} and R_{5ba} together with the carbon atom to which they are attached form a C₃₋₆cycloalkyl or C₃₋₆heterocycloalkyl; and
when Aₐ is —NHC(═O)— or —NHCH₂—:
R_{4ba} and R_{5ba} may additionally be selected from halo, OC₁₋₆haloalkyl, OC₀₋₂alkyleneC₃₋₆cycloalkyl, OC₀₋₂alkyleneC₃₋₆heterocycloalkyl, OC₁₋₄alkyl and NR₂₁ₐR₂₂ₐ;
Ar1a is a 6-membered aryl or heteroaryl;
Ar2a is a 6-membered aryl or heteroaryl and is attached to Ar1a in the para position relative to group Aₐ;
R₁₀ₐ is H, halo, C₁₋₃alkyl, C₁₋₂haloalkyl, OC₁₋₂alkyl, OC₁₋₂haloalkyl or CN;
R₁₁ₐ is H, F, Cl, C₁₋₂alkyl, CF₃, OCH₃ or CN;
R₁₂ₐ is attached to Ar2 in the ortho or meta position relative to Ar1a and R₁₂ₐ is H, halo, C₁₋₄alkyl, C₂₋₄alkenyl, C₀₋₂alkyleneC₃₋₅cycloalkyl, OC₁₋₄alkyl, OC₀₋₂alkyleneC₃₋₅cycloalkyl, C₁₋₄haloalkyl, OC₁₋₄haloalkyl, hydroxy, C₁₋₄alkylOH, SO₂C₁₋₂alkyl, C(O)N(C₁₋₂alkyl)₂, NHC(O)C₁₋₃alkyl or NR₂₃ₐR₂₄ₐ; and
when Aₐ is —NHC(═O)—, —NH— or —NHCH₂—:
R₁₂ₐ may additionally be selected from CN, OCH₂CH₂N(CH₃)₂ and a C₃₋₆heterocycloalkyl comprising one nitrogen located at the point of attachment to Ar2a, or R₁₂ₐ together with a nitrogen atom to which it is attached forms an N-oxide (N⁺—O⁻);
R₁₃ₐ is H or halo;
R₂₁ₐ is H, C₁₋₅alkyl, C(O)C₁₋₅alkyl, C(O)OC₁₋₅alkyl;
R₂₂ₐ is H or CH₃;
R₂₃ₐ is H or C₁₋₂alkyl; and
R₂₄ₐ is H or C₁₋₂alkyl
R₂₉ₐ is C₁₋₃alkyl, C₀₋₂alkyleneC₃₋₅cycloalkyl which cycloalkyl is optionally substituted by CH₃, or CF₃;
R₃₂ₐ is C₁₋₃alkyl and R₃₃ is C₁₋₃alkyl; or
R₃₂ₐ and R₃₃ₐ together with the nitrogen atom to which they are attached form a C₃₋₅heterocycloalkyl;
wherein
R₁ₐ is R₁ₐₐ; and/or
R₄ₐ and R₅ₐ are R₄ₐₐ and R₅ₐₐ; and/or
Aₐ is Aₐₐ; and
wherein when B is (B-bc) and R_{3b3c} is R_{3b}, the compound of formula (I) is a compound of formula (I-b): 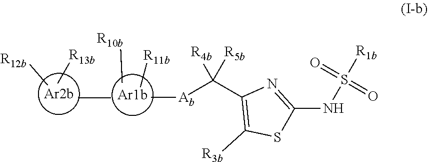
wherein:
A_{b} is A_{ab} or A_{bb};
wherein:
A_{ab} is —NR_{6b}CH₂— or —NR_{6b}—;
A_{bb} is —NR_{6b}C(═O)—;
R_{1b} is R_{1ab} or R_{1bb};
wherein:
R_{1b} is NR_{32b}R_{33b};
R_{1bb} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R_{3b} is H, halo, CH₃, OC₁₋₂alkyl or CF₃;
or R_{3b} together with R_{5bb} forms a 5- or 6-membered cycloalkyl or 5 or 6 membered oxygen-containing heterocycloalkyl;
R_{4b} and R_{5b} are either R_{4b} and R₅ₐ or R_{4bb} and R_{5bb};
wherein:
R_{4ab} and R_{5ab} together with the carbon atom to which they are attached form a C₃₋₆cycloalkyl which is:
substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₄heterocycloalkyl, OC₁₋₃alkyl and NR_{21b}R_{22b}; or
one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl; or
R_{4ab} and R_{5ab} together with the carbon atom to which they are attached form a C₃₋₆heteroycloalkyl wherein one of the carbons of the C₃₋₄heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heteroycloalkyl formed by R_{4ab} and R_{5ab} together with the carbon atom to which they are attached may be substituted by one or two substituents, each substituent being independently selected from the group consisting of C₁₋₃alkyl or OC₁₋₃alkyl; or
R_{4ab} and R_{5ab} together with the carbon atom to which they are attached form a C₃₋₆heterocycloalkyl comprising one nitrogen atom, wherein said nitrogen atom is substituted by —S(O)₂R_{29b}; or
R_{4ab} and R_{5ab} are each independently H, halo, C₁₋₄alkyl, C₀₋₂alkyleneC₃₋₆cycloalkyl, C₀₋₂alkyleneC₃₋₆heterocycloalkyl, OC₁₋₄alkyl, OC₀₋₂alkyleneC₃₋₆cycloalkyl, C₁₋₃alkyleneOC₁₋₃alkyl, C₁₋₆alkylOH, C₁₋₆haloalkyl, OC₁₋₆haloalkyl or NR_{21b}R_{22b},
or R_{4bb} is H and R_{5bb} together with R3b form a 5- or 6-membered cycloalkyl or 5 or 6 membered oxygen-containing heterocycloalkyl,
or R_{4bb} and R_{5bb} together with the carbon atom to which they are attached form a C₃₋₆cycloalkyl or C₃₋₆heterocycloalkyl,
or R_{4bb} is H and R_{5bb} and R_{6b} are a C₂₋₃alkylene chain forming a 5- or 6-membered ring;
or R_{4bb} is O and R_{5bb} is absent;
R_{6b} is H or C₁₋₃alkyl,
or R_{6b} together with R_{11b} when in the ortho-position to group A_{b} are a C₂alkylene chain forming a 5-membered ring,
or R_{5bb} and R_{6b} are a C₂₋₃alkylene chain forming a 5- or 6-membered ring and R_{4bb} is H;
Ar1b is 6-membered aryl or heteroaryl;
Ar2b is a 6-membered aryl or heteroaryl and is attached to Ar1b in the para position relative to group A_{b};
R_{10b} is H, halo, C₁₋₃alkyl, OC₁₋₂alkyl, C₁₋₂haloalkyl, OC₁₋₂haloalkyl or CN;
R_{11b} is H, F, Cl, CH₃, ethyl, OCH₃, CF₃, OCF₃ or CN,
or R_{11b}, when in the ortho-position to group A_{b}, together with R_{6b} are a C₂alkylene chain forming a 5-membered ring;
R_{12b} is attached to Ar2b in the ortho or meta position relative to Ar1b and R_{12b} is H, halo, C₁₋₄alkyl, C₂₋₄alkynyl, C₀₋₂alkyleneC₃₋₅cycloalkyl, OC₁₋₄alkyl, OC₀₋₂alkyleneC₃₋₅cycloalkyl, OCH₂CH₂N(CH₃)₂, OH, C₁₋₄alkylOH, CN, C₁₋₃alkyleneOC₁₋₃alkyl, C₁₋₄haloalkyl, OC₁₋₄haloalkyl, C(═O)C₁₋₂alkyl, NR_{23b}R_{24b}, SO₂C₁₋₄alkyl, SOC₁₋₄alkyl, SC₁₋₄alkyl, SH, C(O)N(CH₃)₂, NHC(O)C₁₋₃alkyl, C₃₋₆heterocycloalkyl comprising one nitrogen located at the point of attachment to Ar2b, or R_{12b} together with a nitrogen atom to which it is attached forms an N-oxide (N⁺—O⁻);
R_{13b} is H, halo, CH₃ or OCH₃;
R_{21b} is H, C₁₋₅alkyl, C(O)C₁₋₅alkyl, C(O)OC₁₋₅alkyl;
R_{22b} is H or CH₃;
R_{23b} is H or C₁₋₂alkyl;
R_{24b} is H or C₁₋₂alkyl;
R_{29b} is C₁₋₃alkyl, C₀₋₂alkyleneC₃₋₅cycloalkyl which cycloalkyl is optionally substituted by CH₃, or CF₃; and
R_{32b} is C₁₋₃alkyl and R_{33b} is C₁₋₃alkyl; or
R_{32b} and R_{33b} together with the nitrogen atom to which they are attached form a C₃₋₅heterocycloalkyl;
wherein:
R_{1b} is R_{1ab}; and/or
R_{4b} and R_{5b} are R_{4ab} and R_{5ab}; and/or
A_{b} is A_{ab}; or
wherein when B is (B-bc) and R_{3b3c} is R_{3c}, the compound of formula (I) is a compound of formula (I-c): 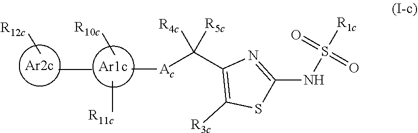
wherein:
A_{c} is A_{ac} or A_{bc};
A_{ac} is —CH₂NR_{6c}—;
A_{bc} is —C(═O)NR₆—;
R_{1c} is R_{1ac} or R_{1bc};
wherein:
R_{1ac} is NR_{32c}R_{33c};
R_{1bc} is C₁₋₅alkyl, C₀₋₂alkyleneC₃₋₅cycloalkyl which cycloalkyl is optionally substituted by CH₃, C₁₋₃alkyleneOC₁₋₂alkyl, or CF₃;
R_{3c} is H, CH₃, halo, OC₁₋₂alkyl or CF₃;
R_{4c} and R_{5c} are either R_{4ac} and R_{5ac} or R_{4bc} and R_{5bc};
wherein:
R_{4ac} and R_{5ac} together with the carbon atom to which they are attached form a C₃₋₆cycloalkyl which is:
substituted by one or two substituents, each substituent being independently selected from the group consisting of C₁₋₃alkyl, oxo, OH, C₁₋₃alkylOH, C₁₋₃haloalkyl, C₀₋₂alkyleneC₃₋₆cycloalkyl, C₀₋₂alkyleneC₃₋₆heterocycloalkyl, C₁₋₃alkyleneOC₁₋₃alkyl, halo, OC₁₋₃haloalkyl, OC₀₋₂alkyleneC₃₋₆cycloalkyl, OC₀₋₂alkyleneC₃₋₆heterocycloalkyl, OC₁₋₃alkyl and NR_{21c}R_{22c}; or
one of the carbons of the C₃₋₆cycloalkyl is a spiro centre such that a spirocyclic ring system is formed by the C₃₋₆cycloalkyl ring and a further C₃₋₆cycloalkyl ring or a C₃₋₆heterocycloalkyl ring, and wherein the C₃₋₆cycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl; or
R_{4ac} and R_{5ac} together with the carbon atom to which they are attached form a C₃₋₆heteroycloalkyl wherein one of the carbons of the C₃₋₆heterocycloalkyl is a spiro centre such that a spirocyclic ring system is formed by the C₃₋₆heterocycloalkyl ring and a further C₃₋₆cycloalkyl ring or a C₃₋₆heterocycloalkyl ring, and wherein the C₃₋₆heteroycloalkyl formed by R_{4ac} and R_{5ac} together with the carbon atom to which they are attached may be substituted by one or two substituents, each substituent being independently selected from the group consisting of C₁₋₃alkyl or OC₁₋₃alkyl; or
R_{4ac} and R_{5ac} together with the carbon atom to which they are attached form a C₃₋₆heterocycloalkyl comprising one nitrogen atom, wherein said nitrogen atom is substituted by —S(O)₂R_{29c}; or
R_{4bc} and R_{5bc} are each independently H, C₁₋₄alkyl, C₀₋₂alkyleneC₃₋₆cycloalkyl, C₀₋₂alkyleneC₃₋₆heterocycloalkyl, C₁₋₃alkyleneOC₁₋₃alkyl, C₁₋₄alkylOH or C₁₋₆haloalkyl,
or R_{4bc} and R_{5bc} together with the carbon atom to which they are attached form a C₃₋₆cycloalkyl or C₃₋₆heterocycloalkyl ring;
R_{6c} is H or C₁₋₃alkyl;
Ar1c is a 6-membered aryl or heteroaryl;
Ar2c is a 6-membered aryl or heteroaryl and is attached to Ar1c in the para position relative to group A_{c};
R_{10c} is H, halo, C₁₋₃alkyl, OC₁₋₂alkyl, C₁₋₂haloalkyl, OC₁₋₂haloalkyl or CN;
R_{11c} is H, F, Cl, CH₃, ethyl, OCH₃, CF₃, OCF₃ or CN;
R_{12c} is attached to Ar2c in the meta or ortho position relative to Ar1c and R_{12c} is H, halo, C₁₋₄alkyl, C₂₋₄alkynyl, C(═O)C₁₋₂alkyl, C₀₋₂alkyleneC₃₋₅cycloalkyl, OC₁₋₄alkyl, C₁₋₃alkyleneOC₁₋₃alkyl, C₁₋₄haloalkyl, OC₁₋₄haloalkyl, CN, OC₀₋₂alkyleneC₃₋₅cycloalkyl, OCH₂CH₂N(CH₃)₂, OH, C₁₋₄alkylOH, NR_{23c}R_{24c}, SO₂CH₃, C(O)N(CH₃)₂, NHC(O)C₁₋₃alkyl, or a C₃₋₆heterocycloalkyl comprising one nitrogen located at the point of attachment to Ar2c, or R_{12c} together with a nitrogen atom to which it is attached forms an N-oxide (N⁺—O⁻);
R_{21c} is H, C₁₋₅alkyl, C(O)C₁₋₅alkyl, C(O)OC₁₋₅alkyl;
R_{22c} is H or CH₃;
R_{23c} is H or C₁₋₂alkyl;
R_{24c} is H or C₁₋₂alkyl;
R_{29c} is C₁₋₃alkyl, C₀₋₂alkyleneC₃₋₅cycloalkyl which cycloalkyl is optionally substituted by CH₃, or CF₃; and
R_{32c} is C₁₋₃alkyl and R_{33c} is C₁₋₃alkyl; or
R_{32c} and R_{33c} together with the nitrogen atom to which they are attached form a C₃₋₅heterocycloalkyl;
wherein:
R_{1c} is R_{1ac}; and/or
R_{4c} and R_{5c} are R_{4ac} and R_{5ac}; and/or
A_{c} is A_{ac};
or a pharmaceutically acceptable salt and/or solvate thereof and/or derivative thereof.

18. The compound, pharmaceutically acceptable salt and/or solvate thereof according to claim 16.

19. The pharmaceutically acceptable salt according to claim 16.

20. The solvate according to claim 16.

21. The compound, pharmaceutically acceptable salt and/or solvate thereof and/or derivative thereof according to claim 16 wherein the compound of formula (I) is a compound of formula (I-a).

22. The compound, pharmaceutically acceptable salt and/or solvate thereof and/or derivative thereof according to claim 16 wherein the compound of formula (I) is a compound of formula (I-b).

23. The compound, pharmaceutically acceptable salt and/or solvate thereof and/or derivative thereof according to claim 16 wherein the compound of formula (I) is a compound of formula (I-c).

24. The compound, pharmaceutically acceptable salt and/or solvate thereof and/or derivative thereof according to claim 21 wherein R₁ₐ is R₁ₐₐ, R_{1b} is R_{1ab} or R_{1c} is R_{1ac}.

25. The compound, pharmaceutically acceptable salt and/or solvate thereof and/or derivative thereof according to claim 21 wherein R₄ₐ and R₅ₐ are R₄ₐₐ and R₅ₐₐ, R_{4b} and R_{5b} are R_{4ab} and R_{5ab} or R_{4c} and R_{5c} are R_{4ac} and R_{5ac}.

26. The compound, pharmaceutically acceptable salt and/or solvate thereof and/or derivative thereof according to claim 21 wherein Aₐ is Aₐₐ, A_{b} is A_{ab} or A_{c} is A_{ac}.

27. A compound, pharmaceutically acceptable salt and/or solvate thereof and/or derivative thereof according to claim 16 which is selected from the group consisting of:
1-(2-(cyclopropanesulfonamido)pyrimidin-4-yl)-N-(5-(6-ethoxypyrazin-2-yl)pyridin-2-yl)-4-oxocyclohexanecarboxamide;
1-(2-(cyclopropanesulfonamido)pyrimidin-4-yl)-N-(5-(6-ethoxypyrazin-2-yl)pyridin-2-yl)-4-hydroxycyclohexanecarboxamide;
1-(2-(cyclopropanesulfonamido)pyrimidin-4-yl)-N-(5-(6-ethoxypyrazin-2-yl)pyridin-2-yl)-4-hydroxycyclohexanecarboxamide (diastereomer 1);
1-(2-(cyclopropanesulfonamido)pyrimidin-4-yl)-N-(5-(6-ethoxypyrazin-2-yl)pyridin-2-yl)-4-hydroxycyclohexanecarboxamide (diastereomer 2);
1-(2-(cyclopropanesulfonamido)pyrimidin-4-yl)-4-(dimethylamino)-N-(5-(6-ethoxypyrazin-2-yl)pyridin-2-yl)cyclohexane-1-carboxamide;
1-(2-(cyclopropanesulfonamido)pyrimidin-4-yl)-4-(dimethylamino)-N-(5-(6-ethoxypyrazin-2-yl)pyridin-2-yl)cyclohexane-1-carboxamide (diastereomer 1);
1-(2-(cyclopropanesulfonamido)pyrimidin-4-yl)-4-(dimethylamino)-N-(5-(6-ethoxypyrazin-2-yl)pyridin-2-yl)cyclohexane-1-carboxamide (diastereomer 2);
N-(4-(1-((4-(6-Ethoxypyrazin-2-yl)-2-fluorobenzyl)amino)propyl)pyrimidin-2-yl)cyclopropanesulfonamide;
1-(2-(cyclopropanesulfonamido)pyrimidin-4-yl)-N-(5-(6-ethoxypyrazin-2-yl)pyridin-2-yl)-4,4-difluorocyclohexane-1-carboxamide;
8-(2-(cyclopropanesulfonamido)pyrimidin-4-yl)-N-(5-(6-ethoxypyrazin-2-yl)pyridin-2-yl)-1,4-dioxaspiro[4.5]decane-8-carboxamide;
4-(2-((N,N-dimethylsulfamoyl)amino)pyrimidin-4-yl)-N-(5-(6-ethoxypyrazin-2-yl)pyridin-2-yl)tetrahydro-2H-pyran-4-carboxamide;
4-(2-(cyclopropanesulfonamido)pyrimidin-4-yl)-N-(5-(6-ethoxypyrazin-2-yl)pyridin-2-yl)-1-(methylsulfonyl)piperidine-4-carboxamide;
N-(4-(1-(((5-(6-ethoxypyrazin-2-yl)pyridin-2-yl)methyl)amino)cyclopropyl)pyrimidin-2-yl)cyclopropanesulfonamide;
N-(4-(1-((4-(6-ethoxypyrazin-2-yl)-2-fluorobenzyl)amino)cyclopropyl)pyrimidin-2-yl)cyclopropanesulfonamide;
N-(4-(4-(((4-(6-ethoxypyrazin-2-yl)phenyl)amino)methyl)tetrahydro-2H-pyran-4-yl)pyrimidin-2-yl)cyclopropanesulfonamide;
2-(2-(cyclopropanesulfonamido)pyrimidin-4-yl)-N-(5-(6-ethoxypyrazin-2-yl)pyridin-2-yl)-5,8-dioxaspiro[3.4]octane-2-carboxamide;
1-(2-(cyclopropanesulfonamido)pyrimidin-4-yl)-N-(5-(6-ethoxypyrazin-2-yl)pyridin-2-yl)-4-methoxycyclohexane-1-carboxamide;
N-(4-(1-((4-(6-ethoxypyrazin-2-yl)phenyl)amino)propyl)pyrimidin-2-yl)cyclopropanesulfonamidearboxamide;
4-(2-(Cyclopropanesulfonamido)pyrimidin-4-yl)-N-(5-(6-ethoxypyrazin-2-yl)pyridin-2-yl)-1-(2-methoxyacetyl)piperidine-4-carboxamide;
4-(2-(Cyclopropanesulfonamido)pyrimidin-4-yl)-N-(5-(6-ethoxypyrazin-2-yl)pyridin-2-yl)-1-(ethylsulfonyl)piperidine-4-carboxamide;
4-(2-(Cyclopropanesulfonamido)pyrimidin-4-yl)-1-(cyclopropylsulfonyl)-N-(5-(6-ethoxypyrazin-2-yl)pyridin-2-yl)piperidine-4-carboxamide;
4-(2-(Cyclopropanesulfonamido)pyrimidin-4-yl)-1-(N,N-dimethylsulfamoyl)-N-(5-(6-ethoxypyrazin-2-yl)pyridin-2-yl)piperidine-4-carboxamide;
4-(2-(Cyclopropanesulfonamido)pyrimidin-4-yl)-N-(5-(6-ethoxypyrazin-2-yl)pyridin-2-yl)-1-((trifluoromethyl)sulfonyl)piperidine-4-carboxamide;
4-(2-(Cyclopropanesulfonamido)pyrimidin-4-yl)-N-(5-(6-ethoxypyrazin-2-yl)pyridin-2-yl)-1-((1-methyl-1H-pyrazol-3-yl)sulfonyl)piperidine-4-carboxamide;
1-(cyanomethyl)-4-(2-(cyclopropanesulfonamido)pyrimidin-4-yl)-N-(5-(6-ethoxypyrazin-2-yl)pyridin-2-yl)piperidine-4-carboxamide;
ethyl 2-(4-(2-(cyclopropanesulfonamido)pyrimidin-4-yl)-4-((5-(6-ethoxypyrazin-2-yl)pyridin-2-yl)carbamoyl)piperidin-1-yl)acetate;
N-(5-(6-ethoxypyrazin-2-yl)pyridin-2-yl)-4-(2-(ethylsulfonamido)pyrimidin-4-yl)-1-(2-methoxyacetyl)piperidine-4-carboxamide;
N-(5-(6-ethoxypyrazin-2-yl)pyridin-2-yl)-4-(2-(ethylsulfonamido)pyrimidin-4-yl)-1-(methylsulfonyl)piperidine-4-carboxamide;
N-(5-(6-ethoxypyrazin-2-yl)pyridin-2-yl)-4-(2-(ethylsulfonamido)pyrimidin-4-yl)-1-(ethylsulfonyl)piperidine-4-carboxamide;
1-(Cyclopropylsulfonyl)-N-(5-(6-ethoxypyrazin-2-yl)pyridin-2-yl)-4-(2-(ethylsulfonamido)pyrimidin-4-yl)piperidine-4-carboxamide;
N-(5-(6-ethoxypyrazin-2-yl)pyridin-2-yl)-4-(2-(ethylsulfonamido)pyrimidin-4-yl)-1-((1-methyl-1H-pyrazol-3-yl)sulfonyl)piperidine-4-carboxamide;
1-(2-(Cyclopropanesulfonamido)pyrimidin-4-yl)-N-(5-(6-cyclopropylpyrazin-2-yl)pyridin-2-yl)-4-methoxycyclohexane-1-carboxamide (diastereomer 1);
1-(2-(Cyclopropanesulfonamido)pyrimidin-4-yl)-N-(5-(6-cyclopropylpyrazin-2-yl)pyridin-2-yl)-4-methoxycyclohexane-1-carboxamide (diastereomer 2);
1-(2-(Cyclopropanesulfonamido)pyrimidin-4-yl)-N-(5-(6-ethoxypyrazin-2-yl)pyridin-2-yl)-4-(pyrrolidin-1-yl)cyclohexane-1-carboxamide (diastereomer 1);
1-(2-(Cyclopropanesulfonamido)pyrimidin-4-yl)-N-(5-(6-ethoxypyrazin-2-yl)pyridin-2-yl)-4-(pyrrolidin-1-yl)cyclohexane-1-carboxamide (diastereomer 2);
4-amino-1-(2-(cyclopropanesulfonamido)pyrimidin-4-yl)-N-(5-(6-ethoxypyrazin-2-yl)pyridin-2-yl)cyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-morpholinocyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-morpholinocyclohexane-1-carboxamide (diastereomer 2);
1-(2-(cyclopropanesulfonamido)pyrimidin-4-yl)-N-(5-(6-ethoxypyrazin-2-yl)pyridin-2-yl)-4-(methyl(oxetan-3-yl)amino)cyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-((2-methoxyethyl)(methyl)amino)cyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-((2-methoxyethyl)(methyl)amino)cyclohexane-1-carboxamide (diastereomer 2);
1-(2-(cyclopropanesulfonamido)pyrimidin-4-yl)-4-((2,2-difluoroethyl)(methyl)amino)-N-(5-(6-ethoxypyrazin-2-yl)pyridin-2-yl)cyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-(4-methylpiperazin-1-yl)cyclohexane-1-carboxamide (diastereomer 1);
1-(2-(cyclopropanesulfonamido)pyrimidin-4-yl)-N-(5-(6-ethoxypyrazin-2-yl)pyridin-2-yl)-4-(4-methylpiperazin-1-yl)cyclohexane-1-carboxamide (diastereomer 2);
4-(6-(cyclopropanesulfonamido)pyrazin-2-yl)-N-(5-(6-ethoxypyrazin-2-yl)pyridin-2-yl)-1-(methylsulfonyl)piperidine-4-carboxamide;
4-(4-(cyclopropanesulfonamido)pyrimidin-2-yl)-N-(5-(6-cyclopropylpyrazin-2-yl)pyridin-2-yl)-1-(methylsulfonyl)piperidine-4-carboxamide;
4-(4-(cyclopropanesulfonamido)pyrimidin-2-yl)-N-(5-(6-ethoxypyrazin-2-yl)pyridin-2-yl)-1-(methylsulfonyl)piperidine-4-carboxamide;
4-(4-(cyclopropanesulfonamido)pyrimidin-2-yl)-N-(5-(6-cyclopropylpyrazin-2-yl)pyridin-2-yl)-1-(ethylsulfonyl)piperidine-4-carboxamide;
4-(4-(cyclopropanesulfonamido)pyrimidin-2-yl)-N-(5-(6-ethoxypyrazin-2-yl)pyridin-2-yl)-1-(ethylsulfonyl)piperidine-4-carboxamide;
N-(4-(1-((2-fluoro-4-(pyridin-3-yl)phenyl)amino)-2-methylpropan-2-yl)thiazol-2-yl)cyclopropanesulfonamide;
N-(4-(2-((4-(6-Ethoxypyrazin-2-yl)-2-fluorobenzyl)amino)propan-2-yl)thiazol-2-yl)cyclopropanesulfonamide; and
N-(4-(2-(((5-(6-ethoxypyrazin-2-yl)pyridin-2-yl)methyl)amino)propan-2-yl)thiazol-2-yl)cyclopropanesulfonamide;
and pharmaceutically acceptable salts and/or solvates and/or derivatives of any one thereof.

28. The compound according to claim 27, which is N-(4-(1-((2-fluoro-4-(pyridin-3-yl)phenyl)amino)-2-methylpropan-2-yl)thiazol-2-yl)cyclopropanesulfonamide, or a pharmaceutically acceptable salt and/or solvate thereof and/or derivative thereof.

29. A pharmaceutical composition comprising the compound, pharmaceutically acceptable salt and/or solvate thereof and/or derivative thereof according to claim 16.

30. A method of treating or preventing a disease associated with the reduction of T-cell and/or B-cell proliferation in a subject; inflammatory skin diseases such as psoriasis or lichen planus; acute and/or chronic GVHD such as steroid resistant acute GVHD; acute lymphoproliferative syndrome (ALPS); systemic lupus erythematosus, lupus nephritis or cutaneous lupus; transplantation; myasthenia gravis, multiple sclerosis or scleroderma/systemic sclerosis; cancer; or a method of enhancing recovery from vascular injury or surgery and reducing morbidity and mortality associated with neointima and restenosis in a subject, said methods comprising administering to a subject in need thereof an effective amount of a compound, pharmaceutically acceptable salt and/or solvate and/or derivative thereof according to claim 16.

31. The method according to claim 30, wherein the method is a method for treating cancer, by administering to a subject in need thereof a compound, pharmaceutically acceptable salt and/or solvate thereof and/or derivative thereof according to claim 16.

32. The method according to claim 31, wherein the cancer is a haematological cancer, such as Acute myeloid leukemia, Angioimmunoblastic T-cell lymphoma, B-cell acute lymphoblastic leukemia, Sweet Syndrome, T-cell Non-Hodgkins lymphoma (including natural killer/T-cell lymphoma, adult T-cell leukaemia/lymphoma, enteropathy type T-cell lymphoma, hepatosplenic T-cell lymphoma and cutaneous T-cell lymphoma), T-cell acute lymphoblastic leukemia, B-cell Non-Hodgkins lymphoma (including Burkitt lymphoma, diffuse large B-cell lymphoma, Follicular lymphoma, Mantle cell lymphoma, Marginal Zone lymphoma), Hairy Cell Leukemia, Hodgkin lymphoma, Lymphoblastic lymphoma, Lymphoplasmacytic lymphoma, Mucosa-associated lymphoid tissue lymphoma, Multiple myeloma, Myelodysplastic syndrome, Plasma cell myeloma, Primary mediastinal large B-cell lymphoma, chronic myeloproliferative disorders (such as chronic myeloid leukemia, primary myelofibrosis, essential thrombocytemia, polycytemia vera) or chronic lymphocytic leukemia.

33. The method according to claim 31, wherein the cancer is a non-haematological cancer, such as selected from the group consisting of bladder cancer, breast, melanoma, neuroblastoma, malignant pleural mesothelioma, and sarcoma.

34. A compound which is selected from the group consisting of:
a compound of formula (I-a): 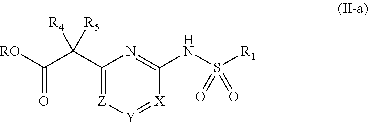
wherein R is H, C₁₋₆alkyl (e.g. methyl and ethyl) or benzyl;
a compound of formula (XX-a): 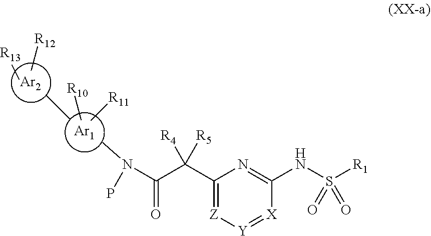
wherein P is a nitrogen protecting group such as para-methoxybenzyl;
a compound of formula (XXIV-a): 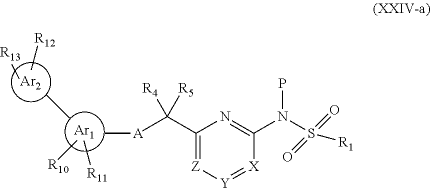
wherein P is a nitrogen protecting group such as para-methoxybenzyl;
a compound of formula (XXXI-a): 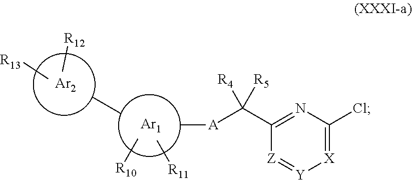
a compound of formula (XXXXII-a): 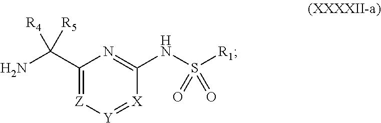
a compound of formula (LI-a): 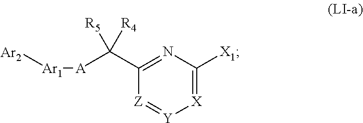
wherein X₁ is Cl or Br;
a compound of formula (LXXIII-a): 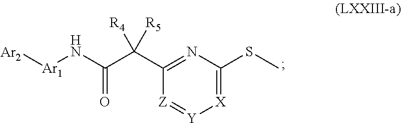
a compound of formula (LXXIV-a): 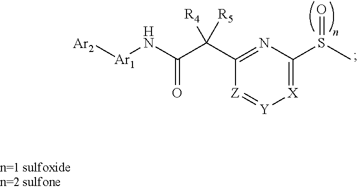
a compound of formula (LXXXIII-a): 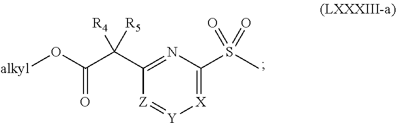
wherein alkyl is C₁₋₄alkyl such as methyl or ethyl, e.g. methyl;
a compound of formula (XXXIV-a): 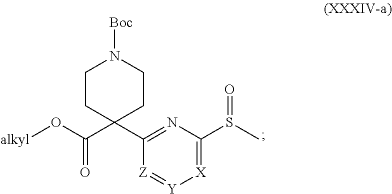
wherein alkyl is C₁₋₄alkyl such as methyl or ethyl, e.g. methyl;
a compound of formula (LVIII-a): 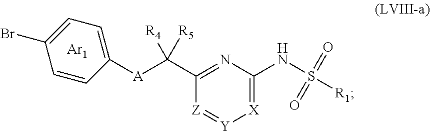
a compound of formula (XXXIII-a): 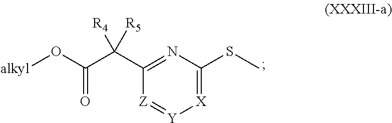
wherein alkyl is C₁₋₄alkyl such as methyl or ethyl, e.g. methyl;
a compound of formula (LXXI-a): 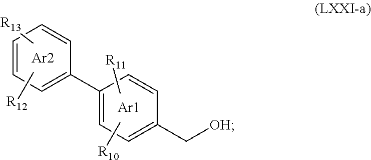
a compound of formula (LXXII-a): 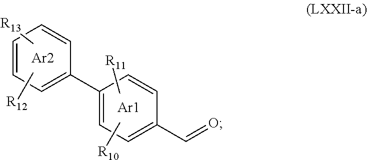
a compound of formula (II-b) 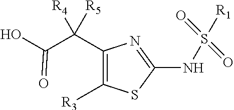
a compound of formula (IV-b) 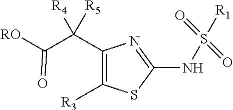
wherein R is C₁₋₄alkyl (e.g. methyl, ethyl) or benzyl;
a compound of formula (II-c): 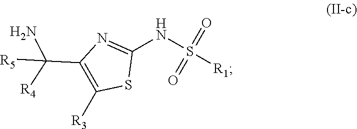
a compound of formula (VIII-c): 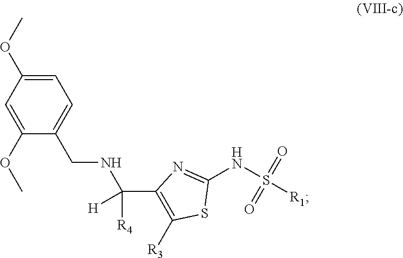
wherein R₁, R₃, R₄, R₅, R₁₀, R₁₁, R₁₂, R₁₃, A, Ar1, Ar2, X, Y and Z are as defined in claim 16;
and salts of any one thereof.

35. A process for the preparation of a compound of formula (I) or a salt, such as a pharmaceutically acceptable salt, and/or solvate thereof and/or derivative thereof, wherein the process comprises the step of:
reacting a compound of formula (I)—ketone or a salt thereof with a compound of formula (I)—amine or a salt thereof: 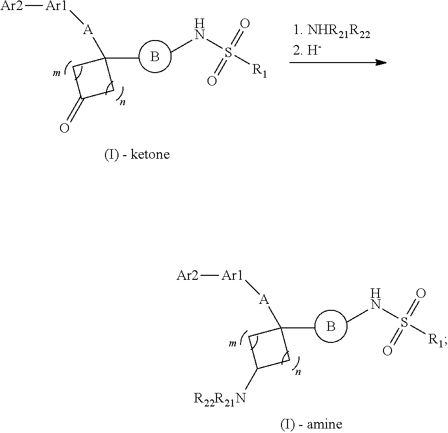
wherein R₁, B, A, Ar1, Ar2, R₂₁, R₂₂, X, Y and Z are as defined in claim 16, and m is 2 and n is 2; or
wherein:
the compound of formula (I) is a compound of formula (I-a) or a salt, such as a pharmaceutically acceptable salt, and/or solvate thereof and/or derivative thereof and wherein the process comprises the step of:
reacting a compound of formula (II) or a salt thereof with a compound of formula (III) or a salt thereof: 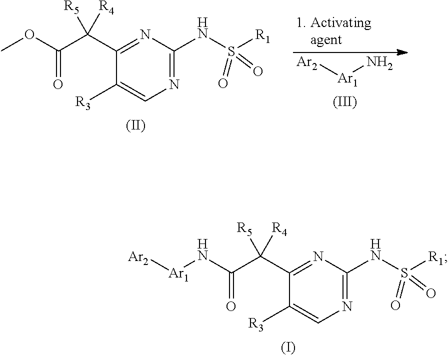
or
reacting a compound of formula (XXXXII) or a salt thereof with a compound of formula (XXXXIII) or a salt thereof: 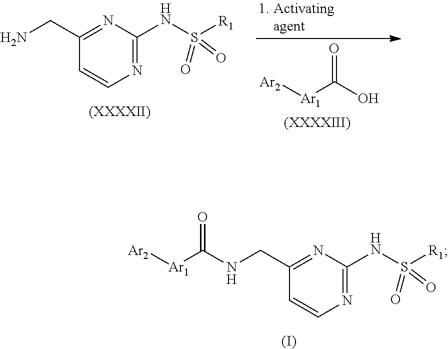
or
reacting a compound of formula (LXXIV) or a salt thereof with a compound of formula (VI) or a salt thereof: 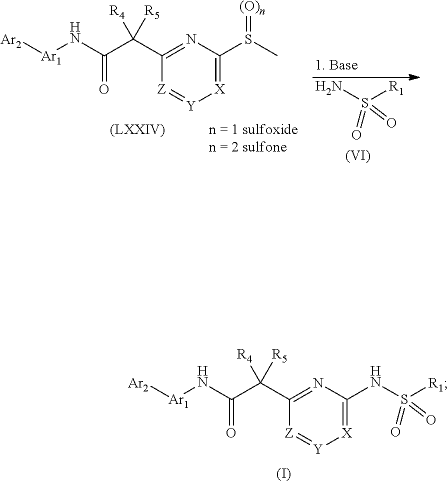
wherein R₁, R₃, R₄, R₅, Ar1, Ar2, X, Y and Z are as defined in claim 16; or
wherein:
the compound of formula (I) is a compound of formula (I-b) or a salt, such as a pharmaceutically acceptable salt, and/or solvate thereof and/or derivative thereof and wherein the process comprises the step of:
reacting a compound of formula (X) or a salt thereof with a compound of formula (XI) or a salt thereof: 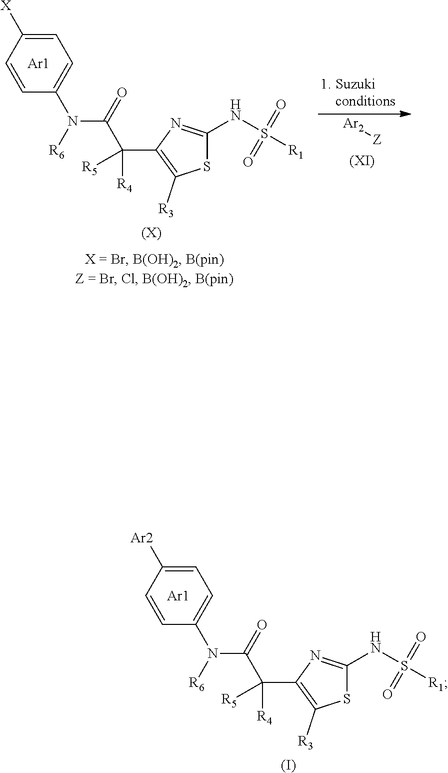
or
reacting a compound of formula (II) or a salt thereof with a compound of formula (III) or a salt thereof: 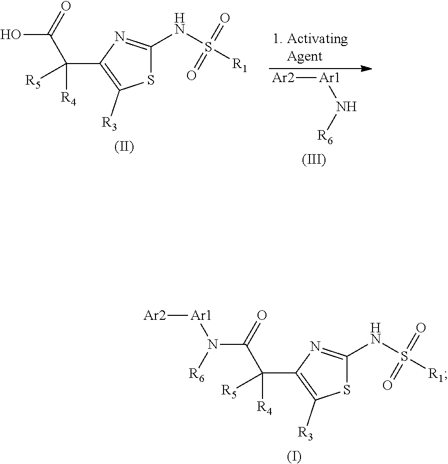
wherein R₁, R₃, R₄, R₅, R₆, Ar1 and Ar2 are as defined in claim 16; or
wherein:
the compound of formula (I) is a compound of formula (I-c) or a salt, such as a pharmaceutically acceptable salt, and/or solvate thereof and/or derivative thereof and wherein the process comprises the step of reacting a compound of formula (XIII) or a salt thereof with a compound of formula (XII) or a salt thereof: 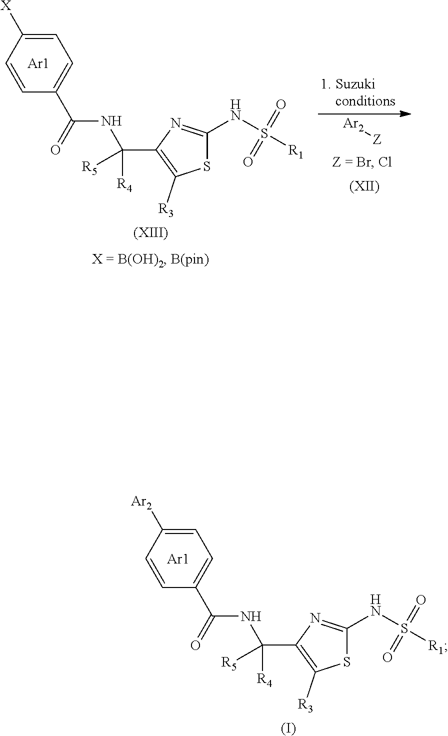
wherein R₁, R₃, R₄, R₅, Ar1 and Ar2 are as defined in claim 16.
